(19) United States
(12) Patent Application Publication
(10) Pub. No.: US 2024/0252642 A1
(43) Pub. Date: Aug. 1, 2024

(54) HYPOIMMUNOGENIC RHD NEGATIVE PRIMARY T CELLS

(71) Applicant: Sana Biotechnology, Inc., Seattle, WA (US)

(72) Inventor: Sonja Schrepfer, San Mateo, CA (US)

(21) Appl. No.: 18/561,682

(22) PCT Filed: May 20, 2022
(86) PCT No.: PCT/US22/30394

§ 371 Date: Nov. 16, 2023

Related U.S. Application Data
(60) Provisional application No. 63/255,803, filed on Oct. 14, 2021.
(60) Provisional application No. 63/190,685, filed on May 19, 2021.

(51) Int. Cl. A61K 39/00; C07K 14/705; C12N 5/0783

(52) U.S. Cl. CPC A61K 39/4634; A61K 39/4611; A61K 39/4631; A61K 39/464412; A61K 39/464413; C07K 14/70596; C12N 5/0636; A61K 2239/26; C12N 2510/00

(57) ABSTRACT

Disclosed herein are hypoimmunogenic T cells having reduced expression of RhD antigen for administering to a patient. In some embodiments, the cells are propagated from a primary T cell or a progeny thereof or are derived from an induced pluripotent stem cell (iPSC). In some embodiments, the cells exogenously express CD47 proteins and exhibit reduced expression of MHC class I proteins, MHC class II proteins, or both. In some embodiments, the cells exogenously express one or more chimeric antigen receptors.

### CROSS-REFERENCE TO RELATED APPLICATIONS

This application claims priority under 35 U.S.C. § 119(e) to U.S. Provisional Application Nos. 63/190,685 filed May 19, 2021, and 63/255,803 filed Oct. 14, 2021, the disclosures of which are herein incorporated by reference in their entireties.

### BACKGROUND

Blood products can be classified into different groups according to the presence or absence of antigens on the surface of every red blood cell in a person's body (ABO Blood Type). The A, B, AB, and A1 antigens are determined by the sequence of oligosaccharides on the glycoproteins of erythrocytes. The genes in the blood group antigen group provide instructions for making antigen proteins. Blood group antigen proteins serve a variety of functions within the cell membrane of red blood cells. These protein functions include transporting other proteins and molecules into and out of the cell, maintaining cell structure, attaching to other cells and molecules, and participating in chemical reactions.

The Rhesus Factor (Rh) blood group is the second most important blood group system, after the ABO blood group system. The Rh blood group system consists of 49 defined blood group antigens, among which five antigens, D, C, c, E, and e, are the most important. RhD status of an individual is normally described with a positive or negative suffix after the ABO type. The terms “Rh factor,” “Rh positive,” “RhD positive,” “Rh negative,” and RhD negative” refer to the RhD antigen only. Antibodies to Rh antigens can be involved in hemolytic transfusion reactions and antibodies to the RhD and Rhc antigens confer significant risk of hemolytic disease of the fetus and new born. ABO antibodies develop in early life in every human. However, rhesus antibodies in RhD− humans typically develop only when the person is sensitized. This can occur, for example, by giving birth to an RhD+ baby or by receiving an RhD+ blood transfusion.

A, B, H, and Rh antigens are major determinants of histocompatibility between donor and recipient for blood, tissue and cellular transplantation. A glycosyltransferase activity encoded by the ABO gene is responsible for producing A, B, AB, O histo-blood group antigens, which are displayed on the surface of cells. Group A individuals encode an ABO gene product with specificity to produce α(1,3)N-acetylgalactosaminyltransferase activity and group B individuals with specificity to produce α(1, 3) galactosyltransferase activity. Type O individuals do not produce a functional galactosyltransferase at all and thus do not produce either modification. Type AB individuals harbor one copy of each and produce both types of modifications. The enzyme products of the ABO gene act on the H antigen as a substrate, and thus type O individuals who lack ABO activity present an unmodified H antigen and are thus often referred to as type O(H).

The H antigen itself is the product of an α(1,2)fucosyltransferase enzyme, which is encoded by the FUT1 gene. In very rare individuals there exists a loss of the H antigen entirely as a result of a disruption of the FUT1 gene and no substrate will exist for ABO to produce A or B histo-blood types. These individuals are said to be of the Bombay histo-blood type. The Rh antigen is encoded by the RHD gene, and individuals who are RhD negative harbor a deletion or disruption of the RHD gene.

The availability of cell-lines suitable for therapeutic applications is severely limited and often the available cell lines are not universally histo-compatible with all possible recipients.

There remains a need for novel approaches, compositions and methods for generating histo-blood type cells that are useful for cell therapies.

### SUMMARY

In some embodiments, provided herein is a hypoimmunogenic T cell comprising reduced expression of Rhesus factor D (RhD) antigen and major histocompatibility complex (MHC) class I and/or class II human leukocyte antigens relative to an unaltered or unmodified wild-type cell, and a first exogenous polynucleotide encoding CD47, wherein the hypoimmunogenic T cell is propagated from a primary T cell or a progeny thereof, or is derived from an induced pluripotent stem cell (iPSC) or a progeny thereof.

In some embodiments, the hypoimmunogenic T cell is propagated from a primary T cell or a progeny thereof, wherein the primary T cell or progeny thereof comprises reduced expression of RhD antigen and MHC class I and/or class II human leukocyte antigens relative to an unaltered or unmodified wild-type cell, and a first exogenous polynucleotide encoding CD47.

In some embodiments, the hypoimmunogenic T cell is derived from an iPSC or a progeny thereof, wherein the iPSC or progeny thereof comprises reduced expression of RhD antigen and MHC class I and/or class II human leukocyte antigens relative to an unaltered or unmodified wild-type cell, and a first exogenous polynucleotide encoding CD47.

In some embodiments, provided herein is a non-activated T cell comprising reduced expression of RhD antigen and MHC class I and/or class II human leukocyte antigens relative to an unaltered or unmodified wild-type cell, and a first exogenous polynucleotide encoding CD47, wherein the non-activated T cell is propagated from a primary T cell or a progeny thereof, or is derived from an iPSC or a progeny thereof.

In some embodiments, the non-activated T cell is propagated from a primary T cell or a progeny thereof, wherein the primary T cell or progeny thereof comprises reduced expression of RhD antigen and MHC class I and/or class II human leukocyte antigens relative to an unaltered or unmodified wild-type cell, and a first exogenous polynucleotide encoding CD47.

In some embodiments, the non-activated T cell is derived from an iPSC or a progeny thereof, wherein the iPSC or progeny thereof comprises reduced expression of RhD antigen and MHC class I and/or class II human leukocyte antigens relative to an unaltered or unmodified wild-type cell, and a first exogenous polynucleotide encoding CD47.

In some embodiments, the non-activated T cell is a non-activated hypoimmunogenic cell.

In some embodiments, provided herein is a population of hypoimmunogenic T cells comprising reduced expression of RhD antigen and MHC class I and/or class II human leukocyte antigens relative to an unaltered or unmodified wild-type cell, and a first exogenous polynucleotide encoding CD47, wherein the population of hypoimmunogenic T cells is propagated from primary T cells or progeny thereof, or is derived from an iPSC or a progeny thereof.

In some embodiments, the population of hypoimmunogenic T cells is propagated from a primary T cell or a progeny thereof, wherein the primary T cell or progeny thereof comprises reduced expression of RhD antigen and MHC class I and/or class II human leukocyte antigens relative to an unaltered or unmodified wild-type cell, and a first exogenous polynucleotide encoding CD47.

In some embodiments, the population of hypoimmunogenic T cells is derived from an iPSC or a progeny thereof, wherein the iPSC or progeny thereof comprises reduced expression of RhD antigen and MHC class I and/or class II human leukocyte antigens relative to an unaltered or unmodified wild-type cell, and a first exogenous polynucleotide encoding CD47.

In some embodiments, the hypoimmunogenic T cell, non-activated T cell, or population of hypoimmunogenic T cells does not express MHC class I and/or class II human leukocyte antigens.

In some embodiments, the hypoimmunogenic T cell, non-activated T cell, or population of hypoimmunogenic T cells comprises reduced expression of beta-2-microglobulin (B2M) and/or MHC class II transactivator (CIITA) relative to an unaltered or unmodified wild-type cell.

In some embodiments, wherein the hypoimmunogenic T cell, non-activated T cell, or population of hypoimmunogenic T cells does not express B2M and/or CIITA.

In some embodiments, reduced expression of RhD antigen is caused by a knock out of the RHD gene.

In some embodiments, the hypoimmunogenic T cell, non-activated T cell, or population of hypoimmunogenic T cells does not express RhD antigen.

In some embodiments, the hypoimmunogenic T cell, non-activated T cell, or population of hypoimmunogenic T cells further comprises reduced expression of a T cell receptor relative to an unaltered or unmodified wild-type cell.

In some embodiments, the hypoimmunogenic T cell, non-activated T cell, or population of hypoimmunogenic T cells does not express a T cell receptor.

In some embodiments, the hypoimmunogenic T cell, non-activated T cell, or population of hypoimmunogenic T cells comprises reduced expression of T cell receptor alpha constant (TRAC) and/or T cell receptor beta constant (TRBC).

In some embodiments, the hypoimmunogenic T cell, non-activated T cell, or population of hypoimmunogenic T cells does not express TRAC and/or TRBC.

In some embodiments, the hypoimmunogenic T cell, non-activated T cell, or population of hypoimmunogenic T cells further comprises a second exogenous polynucleotide encoding one or more chimeric antigen receptors (CARs).

In some embodiments, the one or more CARs are selected from the group consisting of a CD19-specific CAR, such that the cell is a CD19 CAR T cell, a CD20-specific CAR, such that the cell is a CD20 CAR T cell, a CD22-specific CAR, such that the cell is a CD22 CAR T cell, and a BCMA-specific CAR such that the cell is a BCMA CAR T cell, or a combination thereof.

In some embodiments, the hypoimmunogenic T cell, non-activated T cell, or population of hypoimmunogenic T cells comprises a CD19-specific CAR and a CD22-specific CAR such that the cell is a CD19/CD22 CAR T cell.

In some embodiments, the CD19-specific CAR and the CD22-specific CAR are encoded by a single bicistronic polynucleotide.

In some embodiments, the CD19-specific CAR and the CD22-specific CAR are encoded by two separate polynucleotides.

In some embodiments, the first and/or second exogenous polynucleotides are inserted into a specific locus of at least one allele of the cell.

In some embodiments, the specific locus is selected from the group consisting of a safe harbor locus, an RHD locus, a B2M locus, a CIITA locus, a TRAC locus, and a TRB locus.

In some embodiments, the polynucleotide encoding CD47 is introduced to the hypoimmunogenic T cell, non-activated T cell, or population of hypoimmunogenic T cells ex vivo from a donor subject.

In some embodiments, the polynucleotide encoding CD47 is introduced to the hypoimmunogenic T cell, non-activated T cell, or population of hypoimmunogenic T cells using a lentiviral vector.

In some embodiments, the polynucleotide encoding CD47 is introduced to the hypoimmunogenic T cell, non-activated T cell, or population of hypoimmunogenic T cells in vivo in the recipient patient.

In some embodiments, the exogenous polynucleotide encoding CD47 is introduced to the hypoimmunogenic T cell, non-activated T cell, or population of hypoimmunogenic T cells by contacting the recipient patient with a composition comprising lentiviral vectors comprising (i) a CD4 binding agent or a CD8 binding agent, and (ii) a polynucleotide encoding CD47, wherein the hypoimmunogenic T cell, non-activated T cell, or population of hypoimmunogenic T cells of the recipient patient are transduced with the lentiviral vectors.

In some embodiments, the polynucleotide encoding CD47 is introduced to the hypoimmunogenic T cell, non-activated T cell, or population of hypoimmunogenic T cells using CRISPR/Cas gene editing.

In some embodiments, the CRISPR/Cas gene editing is carried out ex vivo from a donor subject.

In some embodiments, the CRISPR/Cas gene editing is carried out using a lentiviral vector.

In some embodiments, the CRISPR/Cas gene editing is carried out in vivo in the recipient patient.

In some embodiments, the CRISPR/Cas gene editing is carried out by contacting the recipient patient with a composition comprising lentiviral vectors comprising (i) a CD4 binding agent or a CD8 binding agent, (ii) polynucleotides encoding CRISPR/Cas gene editing components, and (iii) a polynucleotide encoding CD47, wherein the hypoimmunogenic T cell, non-activated T cell, or population of hypoimmunogenic T cells of the recipient patient are transduced with the lentiviral vectors.

In some embodiments, the one or more CARs are introduced to the hypoimmunogenic T cell, non-activated T cell, or population of hypoimmunogenic T cells ex vivo from a donor subject.

In some embodiments, the one or more CARs are introduced to the hypoimmunogenic T cell, non-activated T cell, or population of hypoimmunogenic T cells using a lentiviral vector.

In some embodiments, the one or more CARs are introduced to the hypoimmunogenic T cell, non-activated T cell, or population of hypoimmunogenic T cells in vivo in the recipient patient.

In some embodiments, the one or more CARs are introduced to the hypoimmunogenic T cell, non-activated T cell, or population of hypoimmunogenic T cells by contacting the recipient patient with a composition comprising lentiviral vectors comprising (i) a CD4 binding agent or a CD8 binding agent, and (ii) one or more polynucleotides encoding the one or more CARs, wherein the hypoimmunogenic T cell, non-activated T cell, or population of hypoimmunogenic T cells of the recipient patient are transduced with the lentiviral vectors.

In some embodiments, the one or more CARs are introduced to the hypoimmunogenic T cell, non-activated T cell, or population of hypoimmunogenic T cells using CRISPR/Cas gene editing.

In some embodiments, the CRISPR/Cas gene editing is carried out ex vivo from a donor subject.

In some embodiments, the CRISPR/Cas gene editing is carried out using a lentiviral vector.

In some embodiments, the CRISPR/Cas gene editing is carried out in vivo in the recipient patient.

In some embodiments, the CRISPR/Cas gene editing is carried out by contacting the recipient patient with a composition comprising lentiviral vectors comprising (i) a CD4 binding agent or a CD8 binding agent, (ii) polynucleotides encoding CRISPR/Cas gene editing components, and (iii) one or more polynucleotides encoding the one or more CARs, wherein the hypoimmunogenic T cell, non-activated T cell, or population of hypoimmunogenic T cells of the recipient patient are transduced with the lentiviral vectors.

In some embodiments, the hypoimmunogenic T cell, non-activated T cell, or population of hypoimmunogenic T cells is propagated from a primary T cell or a progeny thereof, wherein the primary T cell is isolated from a donor subject that is Rhesus factor (Rh) negative.

In some embodiments, the hypoimmunogenic T cell, non-activated T cell, or population of hypoimmunogenic T cells is derived from an iPSC or a progeny thereof, wherein the iPSC or a progeny thereof is derived from a host cell isolated from a donor subject that is RhD negative.

In some embodiments, the hypoimmunogenic T cell, non-activated T cell, or population of hypoimmunogenic T cells is propagated from a primary T cell or a progeny thereof, wherein the primary T cell or a progeny thereof is isolated from a donor subject that is RhD positive and is genetically engineered to have reduced expression of RhD antigen.

In some embodiments, the primary T cell or a progeny thereof is genetically engineered to not express RhD antigen.

In some embodiments, the hypoimmunogenic T cell, non-activated T cell, or population of hypoimmunogenic T cells is derived from an iPSC or a progeny thereof, wherein the iPSC or a progeny thereof is isolated from a donor subject that is RhD positive and is genetically engineered to have reduced expression of RhD antigen.

In some embodiments, the iPSC or a progeny thereof is genetically engineered to not express RhD antigen.

In some embodiments, the hypoimmunogenic T cell, non-activated T cell, or population of hypoimmunogenic T cells is propagated from a pool of primary T cells or progeny thereof, wherein the pool of primary T cells is isolated from one or more donor subjects different from the recipient patient, wherein the one or more donor subjects optionally comprise either one or more subjects that are RhD positive, one or more subjects that are RhD negative, or a mixture of subjects that are RhD positive and subjects that are RhD negative.

In some embodiments, the hypoimmunogenic T cell, non-activated T cell, or population of hypoimmunogenic T cells is derived from a pool of iPSCs or progeny thereof, wherein the pool of iPSCs is derived from host cells isolated from one or more donor subjects different from the recipient patient, wherein the one or more donor subjects optionally comprise either one or more subjects that are RhD positive, one or more subjects that are RhD negative, or a mixture of subjects that are RhD positive and subjects that are RhD negative.

In some embodiments, the hypoimmunogenic T cell, non-activated T cell, or population of hypoimmunogenic T cells is genetically engineered to have reduced expression of RhD antigen using CRISPR/Cas gene editing.

In some embodiments, the CRISPR/Cas gene editing is carried out ex vivo from a donor subject.

In some embodiments, the CRISPR/Cas gene editing is carried out using a lentiviral vector.

In some embodiments, the CRISPR/Cas gene editing is carried out in vivo in the recipient patient.

In some embodiments, the CRISPR/Cas gene editing is carried out by contacting the recipient patient with a composition comprising lentiviral vectors comprising (i) a CD4 binding agent or a CD8 binding agent, and (ii) polynucleotides encoding CRISPR/Cas gene editing components targeting the RHD locus, wherein the hypoimmunogenic T cell, non-activated T cell, or population of hypoimmunogenic T cells of the recipient patient are transduced with the lentiviral vectors.

In some embodiments, provided herein is a pharmaceutical composition comprising one or more hypoimmunogenic T cell, non-activated T cell, or population of hypoimmunogenic T cells provided herein, and a pharmaceutically acceptable additive, carrier, diluent or excipient.

In some embodiments, the composition comprises one or more populations of cells selected from the group consisting of a population of hypoimmunogenic T cells, a population of non-activated T cells, a population hypoimmunogenic CD19 CAR T cells, and a population of hypoimmunogenic CD22 CAR T cells, and a pharmaceutically acceptable additive, carrier, diluent or excipient.

In some embodiments, provided herein is a hypoimmunogenic T cell, non-activated T cell, or population of hypoimmunogenic T cells provided herein, or a pharmaceutical composition provided herein, for use in the treatment of a disorder in a patient, wherein the patient is RhD sensitized.

In some embodiments, provided herein is a hypoimmunogenic T cell, non-activated T cell, or population of hypoimmunogenic T cells provided herein, or a pharmaceutical composition provided herein, for use in the treatment of a disorder in a patient, wherein the patient is not RhD sensitized.

In some embodiments, provided herein is a use of one or more populations of modified T cells for treating a disorder in a recipient patient, wherein the one or more populations of modified T cells are selected from the group consisting of a population of hypoimmunogenic T cells, a population of non-activated T cells, a population hypoimmunogenic CD19 CAR T cells, and a population of hypoimmunogenic CD22 CAR T cells, wherein the modified T cells comprise reduced expression of RhD antigen and MHC class I and/or class II human leukocyte antigens relative to an unaltered or unmodified wild-type cell, and a first exogenous polynucleotide encoding CD47, wherein the modified T cells are propagated from a primary T cell or a progeny thereof, or are derived from an iPSC or a progeny thereof.

In some embodiments, the modified T cells comprise reduced expression of RhD antigen and MHC class I and class II human leukocyte antigens relative to an unaltered or unmodified wild-type cell, and a first exogenous polynucleotide encoding CD47, wherein the modified T cells are propagated from a primary T cell or a progeny thereof, or are derived from an iPSC or a progeny thereof.

In some embodiments, the modified T cells comprise reduced expression of RHD and B2M and/or CIITA relative to an unaltered or unmodified wild-type cell, and a first exogenous polynucleotide encoding CD47, wherein the modified T cells are propagated from a primary T cell or a progeny thereof, or are derived from an iPSC or a progeny thereof.

In some embodiments, the modified T cells comprise reduced expression of RHD and B2M and CIITA relative to an unaltered or unmodified wild-type cell, and a first exogenous polynucleotide encoding CD47, wherein the modified T cells are propagated from a primary T cell or a progeny thereof, or are derived from an iPSC or a progeny thereof.

In some embodiments, the modified T cells do not express RhD antigen, do not express and MHC class I and/or class II human leukocyte antigens, and comprise a first exogenous polynucleotide encoding CD47, wherein the modified T cells are propagated from a primary T cell or a progeny thereof, or are derived from an iPSC or a progeny thereof.

In some embodiments, the modified T cells do not express RhD antigen, do not express MHC class I human leukocyte antigen, do not express MHC class II human leukocyte antigen, and comprise a first exogenous polynucleotide encoding CD47, wherein the modified T cells are propagated from a primary T cell or a progeny thereof, or are derived from an iPSC or a progeny thereof.

In some embodiments, the modified T cells do not express RHD, do not express B2M and/or CIITA, and comprise a first exogenous polynucleotide encoding CD47, wherein the modified T cells are propagated from a primary T cell or a progeny thereof, or are derived from an iPSC or a progeny thereof.

In some embodiments, the modified T cells do not express RHD, do not express B2M, do not express CIITA, and comprise a first exogenous polynucleotide encoding CD47, wherein the modified T cells are propagated from a primary T cell or a progeny thereof, or are derived from an iPSC or a progeny thereof.

In some embodiments, reduced or lack of expression of RhD antigen is caused by a knock out of the RHD gene.

In some embodiments, the modified T cells further comprise reduced expression of a T cell receptor relative to an unaltered or unmodified wild-type cell.

In some embodiments, the modified T cells do not express a T cell receptor.

In some embodiments, the modified T cells comprise reduced expression of TRAC and/or TRBC.

In some embodiments, the modified T cells do not express TRAC and/or TRBC.

In some embodiments, the modified T cells further comprise a second exogenous polynucleotide encoding one or more CARs.

In some embodiments, the one or more CARs are selected from the group consisting of a CD19-specific CAR, such that the cell is a CD19 CAR T cell, a CD20-specific CAR, such that the cell is a CD20 CAR T cell, a CD22-specific CAR, such that the cell is a CD22 CAR T cell, and a BCMA-specific CAR such that the cell is a BCMA CAR T cell, or a combination thereof.

In some embodiments, the hypoimmunogenic T cell, non-activated T cell, or population of hypoimmunogenic T cells comprises a CD19-specific CAR and a CD22-specific CAR such that the cell is a CD19/CD22 CAR T cell.

In some embodiments, the CD19-specific CAR and the CD22-specific CAR are encoded by a single bicistronic polynucleotide.

In some embodiments, the CD19-specific CAR and the CD22-specific CAR are encoded by two separate polynucleotides.

In some embodiments, the first and/or second exogenous polynucleotides are inserted into a specific locus of at least one allele of the cell.

In some embodiments, the specific locus is selected from the group consisting of a safe harbor locus, an RHD locus, a B2M locus, a CIITA locus, a TRAC locus, and a TRB locus.

In some embodiments, the polynucleotide encoding CD47 is introduced to the hypoimmunogenic T cell, non-activated T cell, or population of hypoimmunogenic T cells ex vivo from a donor subject.

In some embodiments, the polynucleotide encoding CD47 is introduced to the hypoimmunogenic T cell, non-activated T cell, or population of hypoimmunogenic T cells using a lentiviral vector.

In some embodiments, the polynucleotide encoding CD47 is introduced to the hypoimmunogenic T cell, non-activated T cell, or population of hypoimmunogenic T cells in vivo in the recipient patient.

In some embodiments, the exogenous polynucleotide encoding CD47 is introduced to the hypoimmunogenic T cell, non-activated T cell, or population of hypoimmunogenic T cells by contacting the recipient patient with a composition comprising lentiviral vectors comprising (i) a CD4 binding agent or a CD8 binding agent, and (ii) a polynucleotide encoding CD47, wherein the hypoimmunogenic T cell, non-activated T cell, or population of hypoimmunogenic T cells of the recipient patient are transduced with the lentiviral vectors.

In some embodiments, the polynucleotide encoding CD47 is introduced to the hypoimmunogenic T cell, non-activated T cell, or population of hypoimmunogenic T cells using CRISPR/Cas gene editing.

In some embodiments, the CRISPR/Cas gene editing is carried out ex vivo from a donor subject.

In some embodiments, the CRISPR/Cas gene editing is carried out using a lentiviral vector.

In some embodiments, the CRISPR/Cas gene editing is carried out in vivo in the recipient patient.

In some embodiments, the CRISPR/Cas gene editing is carried out by contacting the recipient patient with a composition comprising lentiviral vectors comprising (i) a CD4 binding agent or a CD8 binding agent, (ii) polynucleotides encoding CRISPR/Cas gene editing components, and (iii) a polynucleotide encoding CD47, wherein the hypoimmunogenic T cell, non-activated T cell, or population of hypoimmunogenic T cells of the recipient patient are transduced with the lentiviral vectors.

In some embodiments, the one or more CARs are introduced to the hypoimmunogenic T cell, non-activated T cell, or population of hypoimmunogenic T cells ex vivo from a donor subject.

In some embodiments, the one or more CARs are introduced to the hypoimmunogenic T cell, non-activated T cell, or population of hypoimmunogenic T cells using a lentiviral vector.

In some embodiments, the one or more CARs are introduced to the hypoimmunogenic T cell, non-activated T cell, or population of hypoimmunogenic T cells in vivo in the recipient patient.

In some embodiments, the one or more CARs are introduced to the hypoimmunogenic T cell, non-activated T cell, or population of hypoimmunogenic T cells by contacting the recipient patient with a composition comprising lentiviral vectors comprising (i) a CD4 binding agent or a CD8 binding agent, and (ii) one or more polynucleotides encoding the one or more CARs, wherein the hypoimmunogenic T cell, non-activated T cell, or population of hypoimmunogenic T cells of the recipient patient are transduced with the lentiviral vectors.

In some embodiments, the one or more CARs are introduced to the hypoimmunogenic T cell, non-activated T cell, or population of hypoimmunogenic T cells using CRISPR/Cas gene editing.

In some embodiments, the CRISPR/Cas gene editing is carried out ex vivo from a donor subject.

In some embodiments, the CRISPR/Cas gene editing is carried out using a lentiviral vector.

In some embodiments, the CRISPR/Cas gene editing is carried out in vivo in the recipient patient.

In some embodiments, the CRISPR/Cas gene editing is carried out by contacting the recipient patient with a composition comprising lentiviral vectors comprising (i) a CD4 binding agent or a CD8 binding agent, (ii) polynucleotides encoding CRISPR/Cas gene editing components, and (iii) one or more polynucleotides encoding the one or more CARs, wherein the hypoimmunogenic T cell, non-activated T cell, or population of hypoimmunogenic T cells of the recipient patient are transduced with the lentiviral vectors.

In some embodiments, the modified T cells are propagated from a primary T cell or a progeny thereof, wherein the primary T cell is isolated from a donor subject that is Rhesus factor (Rh) negative.

In some embodiments, the modified T cells are derived from an iPSC or a progeny thereof, wherein the iPSC or a progeny thereof is derived from a host cell isolated from a donor subject that is RhD negative.

In some embodiments, the modified T cells are propagated from a primary T cell or a progeny thereof, wherein the primary T cell or a progeny thereof is isolated from a donor subject that is RhD positive and is genetically engineered to have reduced expression of RhD antigen.

In some embodiments, the primary T cell or a progeny thereof is genetically engineered to not express RhD antigen.

In some embodiments, the modified T cells are derived from an iPSC or a progeny thereof, wherein the iPSC or a progeny thereof is isolated from a donor subject that is RhD positive and is genetically engineered to have reduced expression of RhD antigen.

In some embodiments, the iPSC or a progeny thereof is genetically engineered to not express RhD antigen.

In some embodiments, the modified T cells are propagated from a pool of primary T cells or progeny thereof, wherein the pool of primary T cells is isolated from one or more donor subjects different from the recipient patient, wherein the one or more donor subjects optionally comprise either one or more subjects that are RhD positive, one or more subjects that are RhD negative, or a mixture of subjects that are RhD positive and subjects that are RhD negative.

In some embodiments, the modified T cells are derived from a pool of iPSCs or progeny thereof, wherein the pool of iPSCs is derived from host cells isolated from one or more donor subjects different from the recipient patient, wherein the one or more donor subjects optionally comprise either one or more subjects that are RhD positive, one or more subjects that are RhD negative, or a mixture of subjects that are RhD positive and subjects that are RhD negative.

In some embodiments, the modified T cells are genetically engineered to have reduced expression of RhD antigen using CRISPR/Cas gene editing.

In some embodiments, the CRISPR/Cas gene editing is carried out ex vivo from a donor subject.

In some embodiments, the CRISPR/Cas gene editing is carried out using a lentiviral vector.

In some embodiments, the CRISPR/Cas gene editing is carried out in vivo in the recipient patient.

In some embodiments, the CRISPR/Cas gene editing is carried out by contacting the recipient patient with a composition comprising lentiviral vectors comprising (i) a CD4 binding agent or a CD8 binding agent, and (ii) polynucleotides encoding CRISPR/Cas gene editing components targeting the RHD locus, wherein the modified T cells are transduced with the lentiviral vectors.

In some embodiments, the patient is RhD sensitized.

In some embodiments, the patient is not RhD sensitized.

In some embodiments, provided herein is a method for treating a cancer or a disorder in a recipient patient, comprising administering to the patient a therapeutically effective amount of one or more populations of modified T cells, wherein the one or more populations of modified T cells are selected from the group consisting of a population of hypoimmunogenic T cells, a population of non-activated T cells, a population hypoimmunogenic CD19 CAR T cells, and a population of hypoimmunogenic CD22 CAR T cells, wherein the modified T cells comprise reduced expression of RhD antigen and MHC class I and/or class II human leukocyte antigens relative to an unaltered or unmodified wild-type cell, and a first exogenous polynucleotide encoding CD47, wherein the modified T cells are propagated from a primary T cell or a progeny thereof, or are derived from an iPSC or a progeny thereof.

In some embodiments, the modified T cells comprise reduced expression of RhD antigen and MHC class I and class II human leukocyte antigens relative to an unaltered or unmodified wild-type cell, and a first exogenous polynucleotide encoding CD47, wherein the modified T cells are propagated from a primary T cell or a progeny thereof, or are derived from an iPSC or a progeny thereof.

In some embodiments, the modified T cells comprise reduced expression of RHD and B2M and/or CIITA relative to an unaltered or unmodified wild-type cell, and a first exogenous polynucleotide encoding CD47, wherein the modified T cells are propagated from a primary T cell or a progeny thereof, or are derived from an iPSC or a progeny thereof.

In some embodiments, the modified T cells comprise reduced expression of RHD and B2M and CIITA relative to an unaltered or unmodified wild-type cell, and a first exogenous polynucleotide encoding CD47, wherein the modified T cells are propagated from a primary T cell or a progeny thereof, or are derived from an iPSC or a progeny thereof.

In some embodiments, the modified T cells do not express RhD antigen, do not express and MHC class I and/or class II human leukocyte antigens, and comprise a first exogenous polynucleotide encoding CD47, wherein the modified T cells are propagated from a primary T cell or a progeny thereof, or are derived from an iPSC or a progeny thereof.

In some embodiments, the modified T cells do not express RhD antigen, do not express MHC class I human leukocyte antigen, do not express MHC class II human leukocyte antigen, and comprise a first exogenous polynucleotide encoding CD47, wherein the modified T cells are propagated from a primary T cell or a progeny thereof, or are derived from an iPSC or a progeny thereof.

In some embodiments, the modified T cells do not express RHD, do not express B2M and/or CIITA, and comprise a first exogenous polynucleotide encoding CD47, wherein the modified T cells are propagated from a primary T cell or a progeny thereof, or are derived from an iPSC or a progeny thereof.

In some embodiments, the modified T cells do not express RHD, do not express B2M, do not express CIITA, and comprise a first exogenous polynucleotide encoding CD47, wherein the modified T cells are propagated from a primary T cell or a progeny thereof, or are derived from an iPSC or a progeny thereof.

In some embodiments, provided herein is a method for expanding T cells capable of recognizing and killing tumor cells in a patient, comprising administering to the patient a therapeutically effective amount of one or more populations of modified T cells, wherein the one or more populations of modified T cells are selected from the group consisting of a population of hypoimmunogenic T cells, a population of non-activated T cells, a population hypoimmunogenic CD19 CAR T cells, and a population of hypoimmunogenic CD22 CAR T cells, wherein the modified T cells comprise reduced expression of RhD antigen and MHC class I and/or class II human leukocyte antigens relative to an unaltered or unmodified wild-type cell, and a first exogenous polynucleotide encoding CD47, wherein the modified T cells are propagated from a primary T cell or a progeny thereof, or are derived from an iPSC or a progeny thereof.

In some embodiments, the modified T cells comprise reduced expression of RhD antigen and MHC class I and class II human leukocyte antigens relative to an unaltered or unmodified wild-type cell, and a first exogenous polynucleotide encoding CD47, wherein the modified T cells are propagated from a primary T cell or a progeny thereof, or are derived from an iPSC or a progeny thereof.

In some embodiments, the modified T cells comprise reduced expression of RHD and B2M and/or CIITA relative to an unaltered or unmodified wild-type cell, and a first exogenous polynucleotide encoding CD47, wherein the modified T cells are propagated from a primary T cell or a progeny thereof, or are derived from an iPSC or a progeny thereof.

In some embodiments, the modified T cells comprise reduced expression of RHD and B2M and CIITA relative to an unaltered or unmodified wild-type cell, and a first exogenous polynucleotide encoding CD47, wherein the modified T cells are propagated from a primary T cell or a progeny thereof, or are derived from an iPSC or a progeny thereof.

In some embodiments, the modified T cells do not express RhD antigen, do not express and MHC class I and/or class II human leukocyte antigens, and comprise a first exogenous polynucleotide encoding CD47, wherein the modified T cells are propagated from a primary T cell or a progeny thereof, or are derived from an iPSC or a progeny thereof.

In some embodiments, the modified T cells do not express RhD antigen, do not express MHC class I human leukocyte antigen, do not express MHC class II human leukocyte antigen, and comprise a first exogenous polynucleotide encoding CD47, wherein the modified T cells are propagated from a primary T cell or a progeny thereof, or are derived from an iPSC or a progeny thereof.

In some embodiments, the modified T cells do not express RHD, do not express B2M and/or CIITA, and comprise a first exogenous polynucleotide encoding CD47, wherein the modified T cells are propagated from a primary T cell or a progeny thereof, or are derived from an iPSC or a progeny thereof.

In some embodiments, the modified T cells do not express RHD, do not express B2M, do not express CIITA, and comprise a first exogenous polynucleotide encoding CD47, wherein the modified T cells are propagated from a primary T cell or a progeny thereof, or are derived from an iPSC or a progeny thereof.

In some embodiments, reduced or lack of expression of RhD antigen is caused by a knock out of the RHD gene.

In some embodiments, the modified T cells further comprise reduced expression of a T cell receptor relative to an unaltered or unmodified wild-type cell.

In some embodiments, the modified T cells do not express a T cell receptor.

In some embodiments, the modified T cells comprise reduced expression of TRAC and/or TRBC.

In some embodiments, the modified T cells do not express TRAC and/or TRBC.

In some embodiments, the modified T cells further comprise a second exogenous polynucleotide encoding one or more CARs.

In some embodiments, the one or more CARs are selected from the group consisting of a CD19-specific CAR, such that the cell is a CD19 CAR T cell, a CD20-specific CAR, such that the cell is a CD20 CAR T cell, a CD22-specific CAR, such that the cell is a CD22 CAR T cell, and a BCMA-specific CAR such that the cell is a BCMA CAR T cell, or a combination thereof.

In some embodiments, the hypoimmunogenic T cell, non-activated T cell, or population of hypoimmunogenic T cells comprises a CD19-specific CAR and a CD22-specific CAR such that the cell is a CD19/CD22 CAR T cell.

In some embodiments, the CD19-specific CAR and the CD22-specific CAR are encoded by a single bicistronic polynucleotide.

In some embodiments, the CD19-specific CAR and the CD22-specific CAR are encoded by two separate polynucleotides.

In some embodiments, the first and/or second exogenous polynucleotides are inserted into a specific locus of at least one allele of the cell.

In some embodiments, the specific locus is selected from the group consisting of a safe harbor locus, an RHD locus, a B2M locus, a CIITA locus, a TRAC locus, and a TRB locus.

In some embodiments, the polynucleotide encoding CD47 is introduced to the hypoimmunogenic T cell, non-activated T cell, or population of hypoimmunogenic T cells ex vivo from a donor subject.

In some embodiments, the polynucleotide encoding CD47 is introduced to the hypoimmunogenic T cell, non-activated T cell, or population of hypoimmunogenic T cells using a lentiviral vector.

In some embodiments, the polynucleotide encoding CD47 is introduced to the hypoimmunogenic T cell, non-activated T cell, or population of hypoimmunogenic T cells in vivo in the recipient patient.

In some embodiments, the exogenous polynucleotide encoding CD47 is introduced to the hypoimmunogenic T cell, non-activated T cell, or population of hypoimmunogenic T cells by contacting the recipient patient with a composition comprising lentiviral vectors comprising (i) a CD4 binding agent or a CD8 binding agent, and (ii) a polynucleotide encoding CD47, wherein the hypoimmunogenic T cell, non-activated T cell, or population of hypoimmunogenic T cells of the recipient patient are transduced with the lentiviral vectors.

In some embodiments, the polynucleotide encoding CD47 is introduced to the hypoimmunogenic T cell, non-activated T cell, or population of hypoimmunogenic T cells using CRISPR/Cas gene editing.

In some embodiments, the CRISPR/Cas gene editing is carried out ex vivo from a donor subject.

In some embodiments, the CRISPR/Cas gene editing is carried out in vivo in the recipient patient.

In some embodiments, the CRISPR/Cas gene editing is carried out by contacting the recipient patient with a composition comprising lentiviral vectors comprising (i) a CD4 binding agent or a CD8 binding agent, (ii) polynucleotides encoding CRISPR/Cas gene editing components, and (iii) a polynucleotide encoding CD47, wherein the hypoimmunogenic T cell, non-activated T cell, or population of hypoimmunogenic T cells of the recipient patient are transduced with the lentiviral vectors.

In some embodiments, wherein the one or more CARs are introduced to the hypoimmunogenic T cell, non-activated T cell, or population of hypoimmunogenic T cells ex vivo from a donor subject.

In some embodiments, the one or more CARs are introduced to the hypoimmunogenic T cell, non-activated T cell, or population of hypoimmunogenic T cells using a lentiviral vector.

In some embodiments, the one or more CARs are introduced to the hypoimmunogenic T cell, non-activated T cell, or population of hypoimmunogenic T cells in vivo in the recipient patient.

In some embodiments, the one or more CARs are introduced to the hypoimmunogenic T cell, non-activated T cell, or population of hypoimmunogenic T cells by contacting the recipient patient with a composition comprising lentiviral vectors comprising (i) a CD4 binding agent or a CD8 binding agent, and (ii) one or more polynucleotides encoding the one or more CARs, wherein the hypoimmunogenic T cell, non-activated T cell, or population of hypoimmunogenic T cells of the recipient patient are transduced with the lentiviral vectors.

In some embodiments, the one or more CARs are introduced to the hypoimmunogenic T cell, non-activated T cell, or population of hypoimmunogenic T cells using CRISPR/Cas gene editing.

In some embodiments, the CRISPR/Cas gene editing is carried out ex vivo from a donor subject.

In some embodiments, the CRISPR/Cas gene editing is carried out using a lentiviral vector.

In some embodiments, the CRISPR/Cas gene editing is carried out in vivo in the recipient patient.

In some embodiments, the CRISPR/Cas gene editing is carried out by contacting the recipient patient with a composition comprising lentiviral vectors comprising (i) a CD4 binding agent or a CD8 binding agent, (ii) polynucleotides encoding CRISPR/Cas gene editing components, and (iii) one or more polynucleotides encoding the one or more CARs, wherein the hypoimmunogenic T cell, non-activated T cell, or population of hypoimmunogenic T cells of the recipient patient are transduced with the lentiviral vectors.

In some embodiments, the modified T cells are propagated from a primary T cell or a progeny thereof, wherein the primary T cell is isolated from a donor subject that is Rhesus factor (Rh) negative.

In some embodiments, the modified T cells are derived from an iPSC or a progeny thereof, wherein the iPSC or a progeny thereof is derived from a host cell isolated from a donor subject that is RhD negative.

In some embodiments, the modified T cells are propagated from a primary T cell or a progeny thereof, wherein the primary T cell or a progeny thereof is isolated from a donor subject that is RhD positive and is genetically engineered to have reduced expression of RhD antigen.

In some embodiments, the primary T cell or a progeny thereof is genetically engineered to not express RhD antigen.

In some embodiments, the modified T cells are derived from an iPSC or a progeny thereof, wherein the iPSC or a progeny thereof is isolated from a donor subject that is RhD positive and is genetically engineered to have reduced expression of RhD antigen.

In some embodiments, the iPSC or a progeny thereof is genetically engineered to not express RhD antigen.

In some embodiments, the modified T cells are propagated from a pool of primary T cells or progeny thereof, wherein the pool of primary T cells is isolated from one or more donor subjects different from the recipient patient, wherein the one or more donor subjects optionally comprise either one or more subjects that are RhD positive, one or more subjects that are RhD negative, or a mixture of subjects that are RhD positive and subjects that are RhD negative.

In some embodiments, the modified T cells are derived from a pool of iPSCs or progeny thereof, wherein the pool of iPSCs is derived from host cells isolated from one or more donor subjects different from the recipient patient, wherein the one or more donor subjects optionally comprise either one or more subjects that are RhD positive, one or more subjects that are RhD negative, or a mixture of subjects that are RhD positive and subjects that are RhD negative.

In some embodiments, the modified T cells are genetically engineered to have reduced expression of RhD antigen using CRISPR/Cas gene editing.

In some embodiments, the CRISPR/Cas gene editing is carried out ex vivo from a donor subject.

In some embodiments, the CRISPR/Cas gene editing is carried out using a lentiviral vector.

In some embodiments, the CRISPR/Cas gene editing is carried out in vivo in the recipient patient.

In some embodiments, the CRISPR/Cas gene editing is carried out by contacting the recipient patient with a composition comprising lentiviral vectors comprising (i) a CD4 binding agent or a CD8 binding agent, and (ii) polynucleotides encoding CRISPR/Cas gene editing components targeting the RHD locus, wherein the cells are transduced with the lentiviral vectors.

In some embodiments, the patient is RhD sensitized.

In some embodiments, the patient is not RhD sensitized.

In some embodiments, upon administration, the one or more populations of modified T cells elicits a reduced level of immune activation or no immune activation in the patient.

In some embodiments, upon administration, the one or more populations of modified T cells elicits a reduced level of systemic TH1 activation or no systemic TH1 activation in the patient.

In some embodiments, upon administration, the one or more populations of modified T cells elicits a reduced level of immune activation of peripheral blood mononuclear cells (PBMCs) or no immune activation of PBMCs in the patient.

In some embodiments, upon administration, the one or more populations of modified T cells elicits a reduced level of donor-specific IgG antibodies or no donor specific IgG antibodies against the hypoimmunogenic T cells in the patient.

In some embodiments, upon administration, the one or more populations of modified T cells elicits a reduced level of IgM and IgG antibody production or no IgM and IgG antibody production against the hypoimmunogenic T cells in the patient.

In some embodiments, upon administration, the one or more populations of modified T cells elicits a reduced level of cytotoxic T cell killing or no cytotoxic T cell killing of the hypoimmunogenic T cells in the patient.

In some embodiments, the patient is not administered an immunosuppressive agent at least 3 days or more before or after the administration of the population of hypoimmunogenic T cells.

In some embodiments, provided herein is a method of modifying a hypoimmunogenic T cell such that the modified hypoimmunogenic T cell comprises reduced expression of RhD antigen relative to an unaltered or unmodified wild-type cell, the method comprising contacting a hypoimmunogenic T cell with a composition comprising lentiviral vectors comprising (i) a CD4 binding agent or a CD8 binding agent, and (ii) polynucleotides encoding CRISPR/Cas gene editing components targeting the RHD locus, wherein the hypoimmunogenic T cell is transduced with the lentiviral vectors, the hypoimmunogenic T cell is propagated from a primary T cell or a progeny thereof, or is derived from an iPSC or a progeny thereof, and the hypoimmunogenic T cell comprises reduced expression of MHC class I and/or class II human leukocyte antigens relative to an unaltered or unmodified wild-type cell and a first exogenous polynucleotide encoding CD47.

In some embodiments, the lentiviral vectors further comprise (iii) one or more polynucleotides encoding one or more CARs.

In some embodiments, the polynucleotide encoding the one or more CARs is inserted into the RHD locus of the modified hypoimmunogenic T cell.

In some embodiments, the contacting of the hypoimmunogenic T cell is carried out ex vivo from a donor subject.

In some embodiments, the contacting of the hypoimmunogenic T cell is carried out using a lentiviral vector.

In some embodiments, the contacting of the hypoimmunogenic T cell is carried out in vivo in a recipient patient.

In some embodiments, the recipient patient has a disease or condition.

### BRIEF DESCRIPTION OF THE DRAWINGS

FIG. 1A depicts flow cytometry data measuring RhD antigen levels (CD240D) on the cell surface of CD3+ T cells from five RhD+ donors analyzed after thawing, compared to isotype control.

FIG. 1B depicts flow cytometry data measuring RhD antigen levels (CD240D) on the cell surface of CD3+ T cells from five RhD+ donors analyzed after activation with IL-2, compared to isotype control.

FIG. 1C depicts flow cytometry data measuring RhD antigen levels (CD240D) on the cell surface of CD3+ T cells from two RhD− donors analyzed after thawing, compared to isotype control.

FIG. 2A show graphs depicting the assessment of recognition of T cells from RhD+ donors by NK cells in the presence of an anti-RhD antibody using a real time cell killing monitoring assay (e.g., Xcelligence).

FIG. 2B show graphs depicting the assessment of recognition of T cells from RhD+ donors by macrophages in the presence of an anti-RhD antibody using a real time cell killing monitoring assay (e.g., Xcelligence).

FIG. 2C show graphs depicting the assessment of recognition of T cells from RhD− donors by NK cells (top panels) and macrophages (bottom panels) in the presence of an anti-RhD antibody using a real time cell killing monitoring assay (e.g., Xcelligence).

FIG. 3A show graphs depicting the assessment of killing of T cells from RhD+ donors by complement-dependent cytotoxicity (CDC) in the presence of an anti-RhD antibody using a real time cell killing monitoring assay (e.g., Xcelligence).

FIG. 3B show graphs depicting the assessment of killing of T cells from RhD+ donors by CDC in the absence of the anti-RhD antibody (survival control) using a real time cell killing monitoring assay (e.g., Xcelligence).

FIG. 3C show graphs depicting the assessment of killing of T cells from RhD− donors by CDC in the presence of an anti-RhD antibody (top panels) or in the absence of the anti-RhD antibody (survival control; bottom panels) using a real time cell killing monitoring assay (e.g., Xcelligence).

FIG. 4A shows graphs depicting the assessment of killing of T cells from a first donor (blood type O; RhD+) by NK cells (left column), magrophages (middle column), and CDC (right column), in RhD− serum (top row), RhD+ serum (middle row), or RhD− sensitized serum (bottom row).

FIG. 4B shows graphs depicting the assessment of killing of T cells from a second donor (blood type O); RhD+) by NK cells (left column), magrophages (middle column), and CDC (right column), in RhD− serum (top row), RhD+ serum (middle row), or RhD− sensitized serum (bottom row).

FIG. 4C shows graphs depicting the assessment of killing of T cells from a third donor (blood type O; RhD+) by NK cells (left column), magrophages (middle column), and CDC (right column), in RhD− serum (top row), RhD+ serum (middle row), or RhD− sensitized serum (bottom row).

FIG. 4D shows graphs depicting the assessment of killing of T cells from a fourth donor (blood type O; RhD−) by NK cells (left column), magrophages (middle column), and CDC (right column), in RhD− serum (top row), RhD+ serum (middle row), or RhD− sensitized serum (bottom row).

### DETAILED DESCRIPTION

### I. Introduction

The present technology is related to hypoimmunogenic T cells and non-activated T cells comprising reduced expression of Rhesus factor D (RhD) antigen, populations of the cells, pharmaceutical compositions comprising the cells, and methods of treating disorders and conditions comprising administering therapeutically effective amounts of the cells.

To overcome the problem of a recipient patient's immune rejection of these hypoimmunogenic T cells and non-activated T cells, which are propagated from primary T cells or progeny thereof, or derived from induced pluripotent stem cells (iPSCs) or progeny thereof, the inventors have developed and disclose herein methods for generating and administering the hypoimmunogenic T cells and non-activated T cells such that they are protected from adaptive and innate immune rejection upon administration to a recipient patient. Advantageously, the cells disclosed herein are not rejected by the recipient patient's immune system, regardless of the subject's genetic make-up. Such cells are protected from adaptive and innate immune rejection upon administration to a recipient patient.

In some embodiments, hypoimmunogenic T cells and non-activated T cells outlined herein are not subject to an innate immune cell rejection. In some instances, hypoimmunogenic T cells and non-activated T cells are not susceptible to NK cell-mediated lysis. In some instances, hypoimmunogenic T cells and non-activated T cells are not susceptible to macrophage engulfment. In some embodiments, hypoimmunogenic T cells and non-activated T cells are useful as a source of universally compatible cells or tissues (e.g., universal donor cells or tissues) that are transplanted into a recipient patient with little to no immunosuppressant agent needed. Such hypoimmunogenic T cells and non-activated T cells retain cell-specific characteristics and features upon transplantation.

In some embodiments, provided herein are methods for treating a disorder comprising administering cells (e.g., hypoimmunogenic T cells and non-activated T cells) that evade immune rejection in an RhD sensitized patient recipient. In some instances, differentiated cells produced from the stem cells outlined herein evade immune rejection when repeatedly administered (e.g., transplanted or grafted) to an RhD sensitized patient recipient.

In some embodiments, provided herein are methods for treating a disorder comprising administering cells (e.g., hypoimmunogenic T cells and non-activated T cells) that evade immune rejection in an MHC-mismatched allogenic recipient. In some instances, differentiated cells produced from the stem cells outlined herein evade immune rejection when repeatedly administered (e.g., transplanted or grafted) to an MHC-mismatched allogenic recipient.

In some embodiments, provided herein are T cells derived from primary T cells or progeny thereof that are hypoimmunogenic, and cells derived from iPSCs or progeny thereof that are also hypoimmunogenic. In some embodiments, such hypoimmunogenic T cells and non-activated T cells outlined herein have reduced immunogenicity (such as, at least 2.5%-99% less immunogenicity) compared to unaltered or unmodified wild-type immunogenic cells. In some instances, the hypoimmunogenic T cells lack immunogenicity compared to unaltered or unmodified wild-type T cells. The derivatives or progeny thereof are suitable as universal donor cells for transplantation or engrafting into a recipient patient. In some embodiments, such cells are nonimmunogenic to a subject.

In some embodiments, cells disclosed herein fail to elicit a systemic immune response upon administration to a subject. In some cases, the cells do not elicit immune activation of peripheral blood mononuclear cells and serum factors upon administration to a subject. In some instances, the cells do not activate the immune system. In other words, cells described herein exhibit immune evading characteristics and properties. In some embodiments, cells described herein exhibit immunoprivileged characteristics and properties.

Surprisingly, it was found that T cells express RhD antigen. Further, it was found that macrophages and natural killer cells recognize and kill RhD+ T cells by antibody-dependent cellular toxicity (ADCC) in the presence of anti-RhD antibodies, and that RhD+ T cells were killed by complement-dependent cytotoxicity (CDC) in the presence of anti-RhD antibodies. These surprising findings suggest that the source of hypoimmunogenic donor T cells or non-activated donor T cells should be RhD− or genetically modified to be RhD− to avoid detection and elimination by a recipient's immune system, including macrophages and natural killer cells.

### II. Definitions

As used herein, “immunogenicity” refers to property that allows a substance to induce a detectable immune response (humoral or cellular) when introduced into a subject (e.g., a human subject).

As used herein to characterize a cell, the term “hypoimmunogenic” generally means that such cell is less prone to immune rejection by a subject into which such cells are transplanted. For example, relative to an unaltered or unmodified wild-type cell, such a hypoimmunogenic T cell may be about 2.5%, 5%, 10%, 20%, 30%, 40%, 50%, 60%, 70%, 80%, 90%, 95%, 97.5%, 99% or more less prone to immune rejection by a subject into which such cells are transplanted. In some embodiments, genome editing technologies are used to modulate the expression of MHC I and MHC II genes, and thus, generate a hypoimmunogenic T cell. In some embodiments, a hypoimmunogenic T cell evades immune rejection in an MHC-mismatched allogenic recipient. In some instances, differentiated cells produced from the hypoimmunogenic stem cells outlined herein evade immune rejection when administered (e.g., transplanted or grafted) to an MHC-mismatched allogenic recipient. In some embodiments, a hypoimmunogenic T cell is protected from T cell-mediated adaptive immune rejection and/or innate immune cell rejection.

In some embodiments, the hypoimmunogenic T cells and non-activated T cells described are propagated from a primary T cell or a progeny thereof. As used herein, the term “propagated from a primary T cell or a progeny thereof” encompasses the initial primary T cell that is isolated from the donor subject and any subsequent progeny thereof. As used herein, the term “progeny” encompasses, e.g., a first-generation progeny, i.e. the progeny is directly derived from, obtained from, obtainable from or derivable from the initial primary T cell by, e.g., traditional propagation methods. The term “progeny” also encompasses further generations such as second, third, fourth, fifth, sixth, seventh, or more generations, i.e., generations of cells which are derived from, obtained from, obtainable from or derivable from the former generation by, e.g., traditional propagation methods. The term “progeny.” also encompasses modified cells that result from the modification or alteration of the initial primary T cell or a progeny thereof.

In some embodiments, the hypoimmunogenic T cells and non-activated T cells described are derived from an iPSC or a progeny thereof. As used herein, the term “derived from an iPSC or a progeny thereof” encompasses the initial iPSC that is generated and any subsequent progeny thereof. As used herein, the term “progeny” encompasses, e.g., a first-generation progeny, i.e., the progeny is directly derived from, obtained from, obtainable from or derivable from the initial iPSC by, e.g., traditional propagation methods. The term “progeny” also encompasses further generations such as second, third, fourth, fifth, sixth, seventh, or more generations, i.e., generations of cells which are derived from, obtained from, obtainable from or derivable from the former generation by, e.g., traditional propagation methods. The term “progeny” also encompasses modified cells that result from the modification or alteration of the initial iPSC or a progeny thereof.

Hypoimmunogencity of a cell can be determined by evaluating the immunogenicity of the cell such as the cell's ability to elicit adaptive and innate immune responses. Such immune response can be measured using assays recognized by those skilled in the art. In some embodiments, an immune response assay measures the effect of a hypoimmunogenic T cell on T cell proliferation, T cell activation, T cell killing, NK cell proliferation, NK cell activation, and macrophage activity. In some cases, hypoimmunogenic T cells and derivatives thereof undergo decreased killing by T cells and/or NK cells upon administration to a subject. In some instances, the cells and derivatives thereof show decreased macrophage engulfment compared to an unmodified or wildtype cell. In some embodiments, a hypoimmunogenic T cell elicits a reduced or diminished immune response in a recipient subject compared to a corresponding unmodified wild-type cell. In some embodiments, a hypoimmunogenic T cell is nonimmunogenic or fails to elicit an immune response in a recipient subject.

“Pluripotent stem cells” as used herein have the potential to differentiate into any of the three germ layers: endoderm (e.g., the stomach lining, gastrointestinal tract, lungs, etc.), mesoderm (e.g., muscle, bone, blood, urogenital tissue, etc.) or ectoderm (e.g. epidermal tissues and nervous system tissues). The term “pluripotent stem cells,” as used herein, also encompasses “induced pluripotent stem cells”, or “iPSCs”, “embryonic stem cells”, or “ESCs”, a type of pluripotent stem cell derived from a non-pluripotent cell. In some embodiments, a pluripotent stem cell is produced or generated from a cell that is not a pluripotent cell. In other words, pluripotent stem cells can be direct or indirect progeny of a non-pluripotent cell. Examples of parent cells include somatic cells that have been reprogrammed to induce a pluripotent, undifferentiated phenotype by various means. Such “ESC”, “ESC”, “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009): Huangfu et al., Nature Biotechnol. 26 (7): 795 (2008): Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is outlined below. As used herein, “hiPSCs” are human induced pluripotent stem cells.

“HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with β-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

“Rhesus factor D antigen” or “Rh(D) antigen” or “RhD antigen” or “Rhesus D antigen” or “RhD antigen” or “RHD” and variations thereof refer to the Rh antigen encoded by the RHD gene which may be present on the surface of human red blood cells. Those individuals whose red blood cells have this antigen are usually referred to as “RhD positive” or “RhD+” or “Rh positive” or Rh+,” while those individuals whose red blood cells do not have this antigen are referred to as “RhD negative” or “RhD−” or “Rh negative” or Rh−.”

As used herein, the terms “evade rejection,” “escape rejection,” “avoid rejection,” and similar terms are used interchangeably to refer to genetically or otherwise modified membranous products and cells according to the present technology that are less susceptible to rejection when transplanted into a subject when compared with corresponding products and cells that are not genetically modified according to the technology. In some embodiments, the genetically modified products and cells according to the present technology are less susceptible to rejection when transplanted into a subject when compared with corresponding cells that are ABO blood group or Rh factor mismatched to the subject.

By “allogeneic” herein is meant the genetic dissimilarity of a host organism and a cellular transplant where an immune cell response is generated.

As used herein, the terms “grafting”, “administering,” “introducing”, “implanting” and “transplanting” as well as grammatical variations thereof are used interchangeably in the context of the placement of cells (e.g. cells described herein) into a subject, by a method or route which results in at least partial localization of the introduced cells at a desired site. The cells can be implanted directly to the desired site, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years. In some embodiments, the cells can also be administered (e.g., injected) a location other than the desired site, such as in the brain or subcutaneously, for example, in a capsule to maintain the implanted cells at the implant location and avoid migration of the implanted cells.

As used herein, the term “treating” and “treatment” includes administering to a subject an effective amount of cells described herein so that the subject has a reduction in at least one symptom of the disease or an improvement in the disease, for example, beneficial or desired clinical results. For purposes of this technology, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition but may not be a complete cure for the disease. In some embodiments, one or more symptoms of a condition, disease or disorder are alleviated by at least 5%, at least 10%, at least 20%, at least 30%, at least 40%, or at least 50% upon treatment of the condition, disease or disorder.

The term “effective amount” as used herein means an amount of a pharmaceutical composition which is sufficient to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

The term “pharmaceutically acceptable” as used herein, refers to excipients, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “cancer” as used herein is defined as a hyperproliferation of cells whose unique trait (e.g., loss of normal controls) results in unregulated growth, lack of differentiation, local tissue invasion, and metastasis. With respect to the inventive methods, the cancer can be any cancer, including any of acute lymphocytic cancer, acute myeloid leukemia, alveolar rhabdomyosarcoma, bladder cancer, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and urinary bladder cancer. As used herein, the term “tumor” refers to an abnormal growth of cells or tissues of the malignant type, unless otherwise specifically indicated and does not include a benign type tissue.

The term “chronic infectious disease” refers to a disease caused by an infectious agent wherein the infection has persisted. Such a disease may include hepatitis (A, B, or C), herpes virus (e.g., VZV, HSV-1, HSV-6, HSV-II, CMV, and EBV), and HIV/AIDS. Non-viral examples may include chronic fungal diseases such Aspergillosis, Candidiasis, Coccidioidomycosis, and diseases associated with Cryptococcus and Histoplasmosis. None limiting examples of chronic bacterial infectious agents may be Chlamydia pneumoniae, Listeria monocytogenes, and Mycobacterium tuberculosis. In some embodiments, the disorder is human immunodeficiency virus (HIV) infection. In some embodiments, the disorder is acquired immunodeficiency syndrome (AIDS).

The term “autoimmune disease” refers to any disease or disorder in which the subject mounts a destructive immune response against its own tissues. Autoimmune disorders can affect almost every organ system in the subject (e.g., human), including, but not limited to, diseases of the nervous, gastrointestinal, and endocrine systems, as well as skin and other connective tissues, eyes, blood and blood vessels. Examples of autoimmune diseases include, but are not limited to Hashimoto's thyroiditis, Systemic lupus erythematosus, Sjogren's syndrome, Graves' disease, Scleroderma, Rheumatoid arthritis, Multiple sclerosis, Myasthenia gravis and Diabetes.

In some embodiments, the present technology contemplates treatment of non-sensitized subjects. For example, subjects contemplated for the present treatment methods are not sensitized to or against one or more alloantigens. In some embodiments, the patient is not sensitized from a previous pregnancy or a previous allogeneic transplant (including, for example but not limited to an allogeneic cell transplant, an allogeneic blood transfusion, an allogeneic tissue transplant, and an allogeneic organ transplant). In some embodiments, the one or more alloantigens the patient is not sensitized against comprise RhD antigens, such that the patient is “not RhD sensitized”. In some embodiments, the patient does not exhibit memory B cells and/or memory T cells reactive against the one or more alloantigens. In some embodiments, sensitization could include sensitization to at least a portion of an autologous CAR T cell, such as the CAR expressed by the autologous T cell, and in the present methods the patient is not sensitized against any portion of such autologous CAR T cells.

In some embodiments, the present technology contemplates treatment of sensitized subjects. For example, subjects contemplated for the present treatment methods are sensitized to or against one or more alloantigens. In some embodiments, the patient is sensitized from a previous pregnancy or a previous allogeneic transplant (including, for example but not limited to an allogeneic cell transplant, an allogeneic blood transfusion, an allogeneic tissue transplant, and an allogeneic organ transplant). In some embodiments, the one or more alloantigens the patent is sensitized against comprise RhD antigens, such that the patient is “RhD sensitized”. In some embodiments, the patient exhibits memory B cells and/or memory T cells reactive against the one or more alloantigens.

In some embodiments, the present technology contemplates altering target polynucleotide sequences in any manner which is available to the skilled artisan, e.g., utilizing a TALEN system or RNA-guided transposases. It should be understood that although examples of methods utilizing CRISPR/Cas (e.g., Cas9 and Cas12A) and TALEN are described in detail herein, the technology is not limited to the use of these methods/systems. Other methods of targeting, e.g., B2M, to reduce or ablate expression in target cells known to the skilled artisan can be utilized herein.

The RNA molecule that binds to CRISPR-Cas components and targets them to a specific location within the target DNA is referred to herein as “guide RNA,” “gRNA,” or “small guide RNA” and may also be referred to herein as a “DNA-targeting RNA.” A guide RNA comprises at least two nucleotide segments: at least one “DNA-binding segment” and at least one “polypeptide-binding segment.” By “segment” is meant a part, section, or region of a molecule, e.g., a contiguous stretch of nucleotides of an RNA molecule. The definition of “segment,” unless otherwise specifically defined, is not limited to a specific number of total base pairs. In some embodiments, the targeting is accomplished through hybridization of a portion of the gRNA to DNA (e.g., through the gRNA targeting domain), and by binding of a portion of the gRNA molecule to the RNA-guided nuclease or other effector molecule (e.g., through at least the gRNA tracr). In some embodiments, a gRNA molecule consists of a single contiguous polynucleotide molecule, referred to herein as a “single guide RNA” or “sgRNA” and the like. In some embodiments, a gRNA molecule consists of a single contiguous polynucleotide molecule, e.g. in the case of a Cas12a-based system, referred to herein as a “crRNA.” In other embodiments, a gRNA molecule includes a plurality, usually two, polynucleotide molecules, which are themselves capable of association, usually through hybridization, referred to herein as a “dual guide RNA” or “dgRNA,” and the like. gRNA molecules are described in more detail below, and generally include a targeting domain and a tracr. In other embodiments the targeting domain and tracr are disposed on a single polynucleotide. The guide RNA can be introduced into the target cell as an isolated RNA molecule or is introduced into the cell using an expression vector containing DNA encoding the guide RNA.

The term “guide RNA target” as used herein includes an RNA sequence of each and any of the guide RNA targets described herein and variants thereof which are utilized for gene editing. In some embodiment, the guide RNA target includes a target sequence to which a guide RNA binds, thereby allowing for gene editing of the target sequence. The guide RNA target can correspond to a target sequence and does not include a PAM sequence.

The “DNA-binding segment” (or “DNA-targeting sequence”) of the guide RNA comprises a nucleotide sequence that is complementary to a specific sequence within a target DNA.

The guide RNA can include one or more polypeptide-binding sequences/segments. The polypeptide-binding segment (or “protein-binding sequence”) of the guide RNA interacts with the RNA-binding domain of a Cas protein.

The term “Cas9 molecule,” as used herein, refers to Cas9 wild-type proteins derived from Type II CRISPR-Cas9 systems, modifications of Cas9 proteins, variants of Cas9 proteins, Cas9 orthologs, and combinations thereof.

The term “Cas12a molecule,” as used herein, refers to Cas12a wild-type proteins derived from Type II CRISPR-Cas12a systems, modifications of Cas12a proteins, variants of Cas12a proteins, Cas12a orthologs, and combinations thereof.

The term “donor polynucleotide,” “donor template” and “donor oligonucleotide” are used interchangeably and refer to a polynucleotide that provides a nucleic acid sequence of which at least a portion is intended to be integrated into a selected nucleic acid target site. Generally speaking, a donor polynucleotide is a single-strand polynucleotide or a double-strand polynucleotide. For example, an engineered Type II CRISPR-Cas9 system can be used in combination with a donor DNA template to modify a DNA target sequence in a genomic DNA wherein the genomic DNA is modified to comprise at least a portion of the donor DNA template at the DNA target sequence. In some embodiments, a vector comprises a donor polynucleotide. In other embodiments, a donor polynucleotide is an oligonucleotide.

The term “HDR”, as used herein, refers to homology-directed repair, as used herein, refers to the process of repairing DNA damage using a homologous nucleic acid (e.g., an endogenous homologous sequence, e.g., a sister chromatid, or an exogenous nucleic acid, e.g., a template nucleic acid). HDR typically acts when there has been significant resection at the double strand break, forming at least one single stranded portion of DNA. In a normal cell, HDR typically involves a series of steps such as recognition of the break, stabilization of the break, resection, stabilization of single stranded DNA, formation of a DNA crossover intermediate, resolution of the crossover intermediate, and ligation. In some cases, HDR requires nucleotide sequence homology and uses a donor template (e.g., a donor DNA template) or donor oligonucleotide to repair the sequence wherein the double-strand break occurred (e.g., DNA target sequence). This results in the transfer of genetic information from, for example, the donor template DNA to the DNA target sequence. HDR may result in alteration of the DNA target sequence (e.g., insertion, deletion, mutation) if the donor template DNA sequence or oligonucleotide sequence differs from the DNA target sequence and part or all of the donor template DNA polynucleotide or oligonucleotide is incorporated into the DNA target sequence. In some embodiments, an entire donor template DNA polynucleotide, a portion of the donor template DNA polynucleotide, or a copy of the donor polynucleotide is integrated at the site of the DNA target sequence.

The term “non-homologous end joining” or “NHEJ”, as used herein, refers to ligation mediated repair and/or non-template mediated repair.

The methods of the present technology can be used to alter a target polynucleotide sequence in a cell. The present technology contemplates altering target polynucleotide sequences in a cell for any purpose. In some embodiments, the target polynucleotide sequence in a cell is altered to produce a mutant cell. As used herein, a “mutant cell” refers to a cell with a resulting genotype that differs from its original genotype. In some instances, a “mutant cell” exhibits a mutant phenotype, for example when a normally functioning gene is altered using the CRISPR/Cas systems. In other instances, a “mutant cell” exhibits a wild-type phenotype, for example when a CRISPR/Cas system is used to correct a mutant genotype. In some embodiments, the target polynucleotide sequence in a cell is altered to correct or repair a genetic mutation (e.g., to restore a normal phenotype to the cell). In some embodiments, the target polynucleotide sequence in a cell is altered to induce a genetic mutation (e.g., to disrupt the function of a gene or genomic element).

In some embodiments, the alteration is an indel. As used herein, “indel” refers to a mutation resulting from an insertion, deletion, or a combination thereof. As will be appreciated by those skilled in the art, an indel in a coding region of a genomic sequence will result in a frameshift mutation, unless the length of the indel is a multiple of three. In some embodiments, the alteration is a point mutation. As used herein, “point mutation” refers to a substitution that replaces one of the nucleotides. A CRISPR/Cas system can be used to induce an indel of any length or a point mutation in a target polynucleotide sequence.

As used herein, “knock out” includes deleting all or a portion of the target polynucleotide sequence in a way that interferes with the function of the target polynucleotide sequence. For example, a knock out can be achieved by altering a target polynucleotide sequence by inducing an indel in the target polynucleotide sequence in a functional domain of the target polynucleotide sequence (e.g., a DNA binding domain). Those skilled in the art will readily appreciate how to use the CRISPR/Cas systems to knock out a target polynucleotide sequence or a portion thereof based upon the details described herein.

In some embodiments, the alteration results in a knock out of the target polynucleotide sequence or a portion thereof. Knocking out a target polynucleotide sequence or a portion thereof using a CRISPR/Cas system can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject). For in vivo purposes, knocking out a target polynucleotide sequence in a cell can be useful for treating or preventing a disorder associated with expression of the target polynucleotide sequence (e.g., by knocking out RHD expression in cells that have been transplanted into an RhD negative recipient patient).

By “knock in” herein is meant a process that adds a genetic function to a host cell. This causes increased levels of the knocked in gene product, e.g., an RNA or encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

In some embodiments, the alteration results in reduced expression of the target polynucleotide sequence relative to an unaltered or unmodified wild-type cell.

By “wild-type” or “wt” in the context of a cell means any cell found in nature. However, in the context of a hypoimmunogenic T cell, as used herein, “wild-type” also means a hypoimmunogenic T cell that may contain nucleic acid changes resulting in hypoimmunogenicity but did not undergo the gene editing procedures of the present technology to achieve reduced expression of RhD antigen. In the context of an iPSC or a progeny thereof, “wild-type” also means an iPSC or progeny thereof that may contain nucleic acid changes resulting in pluripotency but did not undergo the gene editing procedures of the present technology to achieve hypoimmunogenicity and/or reduced expression of RhD antigen. In the context of a primary T cell or a progeny thereof, “wild-type” also means a primary T cell or progeny thereof that may contain nucleic acid changes resulting in hypoimmunogenicity but did not undergo the gene editing procedures of the present technology to achieve reduced expression of RhD antigen. In some embodiments, “wild-type” refers to an RhD positive cell. In some embodiments, “wild-type” refers to an RhD positive hypoimmunogenic T cell that may contain nucleic acid changes resulting in hypoimmunogenicity but did not undergo the gene editing procedures described to achieve reduced expression of RhD antigen. In some embodiments, “wild-type” refers to an RhD positive iPSC cell or progeny thereof that may contain nucleic acid changes resulting in pluripotency but did not undergo the gene editing procedures of the present technology to achieve hypoimmunogenicity and/or reduced expression of RhD antigen. In some embodiments, “wild-type” refers to an RhD positive primary T cell or progeny thereof that may contain nucleic acid changes resulting in hypoimmunogenicity but did not undergo the gene editing procedures described to achieve reduced expression of RhD antigen

The terms “decrease,” “reduced,” “reduction,” and “decrease” are all used herein generally to mean a decrease by a statistically significant amount. However, for avoidance of doubt, decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level. In some embodiments, reduced expression of the target polynucleotide sequence results from reduced transcription and/or translation of a coding sequence, including genomic DNA, mRNA, etc., into a polypeptide, or protein. In some embodiments, the reduced transcription and/or translation of the coding sequence is a result of an alteration of the target polynucleotide, including an indel, a point mutation, a knock out, or a knock in.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “exogenous” in intended to mean that the referenced molecule or the referenced polypeptide is introduced into the cell of interest. The polypeptide can be introduced, for example, by introduction of an encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell.

The term “endogenous” refers to a referenced molecule or polypeptide that is present in the cell. Similarly, the term when used in reference to expression of an encoding nucleic acid refers to expression of an encoding nucleic acid contained within the cell and not exogenously introduced.

“Safe harbor locus” as used herein refers to a gene locus that allows safe expression of a transgene or an exogenous gene. Exemplary “safe harbor” loci include, but are not limited to, a CCR5 gene, a CXCR4 gene, a PPP1R12C (also known as AAVS1) gene, an albumin gene, a SHS231 locus, a CLYBL gene, a Rosa gene (e.g., ROSA26), an F3 gene (also known as CD142), a MICA gene, a MICB gene, an LRP1 gene (also known as CD91), a HMGB1 gene, an ABO gene, an RHD gene, a FUT1 gene, and a KDM5D gene (also known as HY). The exogenous gene can be inserted in the CDS region for B2M, CIITA, TRAC, TRBC, CCR5, F3 (i.e., CD142), MICA, MICB, LRP1, HMGB1, ABO, RHD, FUT1, or KDM5D (i.e., HY). The exogenous gene can be inserted in introns 1 or 2 for PPP1R12C (i.e., AAVS1) or CCR5. The exogenous gene can be inserted in exons 1 or 2 or 3 for CCR5. The exogenous gene can be inserted in intron 2 for CLYBL. The exogenous gene can be inserted in a 500 bp window in Ch-4:58,976,613 (i.e., SHS231). The exogenous gene can be insert in any suitable region of the aforementioned safe harbor loci that allows for expression of the exogenous, including, for example, an intron, an exon or a coding sequence region in a safe harbor locus.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

The term “donor subject” refers to an animal, for example, a human from whom cells can be obtained. The “non-human animals” and “non-human mammals” as used interchangeably herein, includes mammals such as rats, mice, rabbits, sheep, cats, dogs, cows, pigs, and non-human primates. The term “donor subject” also encompasses any vertebrate including but not limited to mammals, reptiles, amphibians and fish. However, advantageously, the donor subject is a mammal such as a human, or other mammals such as a domesticated mammal, e.g. dog, cat, horse, and the like, or production mammal, e.g. cow, sheep, pig, and the like.

The term “recipient patient” refers to an animal, for example, a human to whom treatment, including prophylactic treatment, with the cells as described herein, is provided. For treatment of those infections, conditions or disease states, which are specific for a specific animal such as a human patient, the term patient refers to that specific animal. The term “recipient patient” also encompasses any vertebrate including but not limited to mammals, reptiles, amphibians and fish. However, advantageously, the recipient patient is a mammal such as a human, or other mammals such as a domesticated mammal, e.g. dog, cat, horse, and the like, or production mammal, e.g. cow, sheep, pig, and the like.

It is noted that the claims may be drafted to exclude any optional element. As such, this statement is intended to serve as antecedent basis for use of such exclusive terminology as “solely.” “only,” and the like in connection with the recitation of claim elements, or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the present technology. Any recited method may be carried out in the order of events recited or in any other order that is logically possible. Although any methods and materials similar or equivalent to those described herein may also be used in the practice or testing of the present technology, representative illustrative methods and materials are now described.

As described in the present technology, the following terms will be employed, and are defined as indicated below.

Before the present technology is further described, it is to be understood that this technology is not limited to some embodiments described, as such may, of course, vary. It is also to be understood that the terminology used herein is for the purpose of describing some embodiments only, and is not intended to be limiting, since the scope of the present technology will be limited only by the appended claims.

Unless defined otherwise, all technical and scientific terms used herein have the same meaning as commonly understood by one of ordinary skill in the art to which this technology belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the present technology. The upper and lower limits of these smaller ranges may independently be included in the smaller ranges and are also encompassed within the present technology, subject to any specifically excluded limit in the stated range. Where the stated range includes one or both of the limits, ranges excluding either or both of those included limits are also included in the present technology. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the present technology described herein is not entitled to antedate such publication by virtue of prior technology. Further, the dates of publication provided might be different from the actual publication dates, which may need to be independently confirmed.

### III. Detailed Description of the Embodiments

### A. Hypoimmunogenic T Cells

In some embodiments, the present technology disclosed herein is directed to hypoimmunogenic T cells and non-activated T cells propagated from primary T cells or progeny thereof, or derived from induced pluripotent stem cells (iPSCs) or progeny thereof that have reduced expression or lack expression of RhD antigen and MHC class I and/or MHC class II human leukocyte antigens and overexpress CD47. In some embodiments, hypoimmunogenic T cells and non-activated T cells have reduced expression of RhD antigen and MHC class I and/or MHC class II human leukocyte antigens relative to an unaltered or unmodified wild type cell, and overexpress CD47. In some embodiments, hypoimmunogenic T cells and non-activated T cells have reduced expression of RhD antigen and MHC class I and MHC class II human leukocyte antigens relative to an unaltered or unmodified wild type cell, and overexpress CD47. In some embodiments, hypoimmunogenic T cells and non-activated T cells have reduced expression of RHD and B2M and/or CIITA, and overexpress CD47. In some embodiments, hypoimmunogenic T cells and non-activated T cells have reduced expression of RHD, B2M, and CIITA, and overexpress CD47. In some embodiments, hypoimmunogenic T cells and non-activated T cells do not express RhD antigen, do not express MHC class I and/or class II human leukocyte antigens, and overexpress CD47. In some embodiments, hypoimmunogenic T cells and non-activated T cells do not express RhD antigen, do not express MHC class I human leukocyte antigen, do not express MHC class II human leukocyte antigen, and overexpress CD47. In some embodiments, hypoimmunogenic T cells and non-activated T cells do not express RHD, do not express B2M and/or CIITA, and overexpress CD47. In some embodiments, hypoimmunogenic T cells and non-activated T cells do not express RHD, do not express B2M, do not express CIITA, and overexpress CD47. In some embodiments, hypoimmunogenic T cells and non-activated T cells have reduced expression of a T cell receptor relative to an unaltered or unmodified wild type cell. In some embodiments, hypoimmunogenic T cells and non-activated T cells do not express a T cell receptor. In some embodiments, hypoimmunogenic T cells and non-activated T cells have reduced expression of T cell receptor alpha constant (TRAC) and/or T cell receptor beta constant (TRBC) relative to an unaltered or unmodified wild type cell. In some embodiments, hypoimmunogenic T cells and non-activated T cells do not express T cell receptor alpha constant (TRAC) and/or T cell receptor beta constant (TRBC). In some embodiments, hypoimmunogenic T cells and non-activated T cells comprise a second exogenous polynucleotide encoding one or more chimeric antigen receptors (CARs). In some embodiments, the one or more CARs comprise an antigen binding domain that binds to any one selected from the group consisting of CD19, CD20, CD22, and BCMA, or combinations thereof. In some embodiments, the one or more CARs comprise a CD19-specific CAR such that the cell is a “CD19 CAR T cell.” In some embodiments, the one or more CARs comprise a CD22-specific CAR such that the cell is a “CD22 CAR T cell.”

In some embodiments, hypoimmunogenic T cells and non-activated T cells overexpress CD47 and one or more chimeric antigen receptors (CARs), and include a genomic modification of the RHD and the B2M gene. In some embodiments, hypoimmunogenic T cells and non-activated T cells overexpress CD47 and include a genomic modification of the RHD and the CIITA gene. In some embodiments, hypoimmunogenic T cells and non-activated T cells overexpress CD47 and one or more CARs, and include a genomic modification of the RHD and the TRAC gene. In some embodiments, hypoimmunogenic T cells and non-activated T cells overexpress CD47 and one or more CARs, and include a genomic modification of the RHD and the TRB gene. In some embodiments, hypoimmunogenic T cells and non-activated T cells overexpress CD47 and one or more CARs, include a genomic modification of the RHD gene, and include one or more genomic modifications selected from the group consisting of the B2M, CIITA, TRAC, and TRB genes. In some embodiments, hypoimmunogenic T cells and non-activated T cells overexpress CD47 and one or more CARs, and include genomic modifications of the RHD, B2M, CIITA, TRAC, and TRB genes. In some embodiments, the cells are RHD^{-/-}, B2M^{-/-}, CIITA^{-/-}, TRAC^{-/-}, CD47tg cells that also express CARs. In some embodiments, hypoimmunogenic T cells and non-activated T cells are RHD^{-/-}, B2M^{-/-}, CIITA^{-/-}, TRB^{-/-}, CD47tg cells that also express CARs. In some embodiments, the cells are B2M^{-/-}, CIITA^{-/-}, TRAC^{-/-}, TRB^{-/-}, CD47tg cells that also express CARs. In some embodiments, the cells are RHD^{indel/indel}, B2M^{indel/indel}, CIITA^{indel/indel}, TRAC^{indel/indel}. CD47tg cells that also express CARs. In some embodiments, the cells are RHD^{indel/indel}, B2M^{indel/indel}, CIITA^{indel/indel}, TRB^{indel/indel}, CD47tg cells that also express CARs. In some embodiments, the cells are RHD^{indel/indel}, B2M^{indel/indel}, CIITA^{indel/indel}, TRAC^{indel/indel}, TRB^{indel/indel}, CD47tg cells that also express CARs.

In some embodiments, hypoimmunogenic T cells and non-activated T cells are produced by differentiating induced pluripotent stem cells such as hypoimmunogenic induced pluripotent stem cells.

In some embodiments, the engineered or modified cells described are pluripotent stem cells, induced pluripotent stem cells, T cells differentiated from such pluripotent stem cells and induced pluripotent stem cells, or primary T cells. Non-limiting examples of primary T cells include CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells. In some embodiments, the primary T cells are selected from a group that includes cytotoxic T-cells, helper T-cells, memory T-cells, regulatory T-cells, tumor infiltrating lymphocytes, and combinations thereof.

In some embodiments, the primary T cells are from a pool of primary T cells from one or more donor subjects that are different than the recipient patient (e.g., the patient administered the cells). The primary T cells can be obtained from 1, 2, 3, 4, 5, 6, 7, 8, 9, 10, 20, 50, 100 or more donor subjects and pooled together. The primary T cells can be obtained from 1 or more, 2 or more, 3 or more, 4 or more, 5 or more, 6 or more, 7 or more, 8 or more, 9 or more, 10, or more 20 or more, 50 or more, or 100 or more donor subjects and pooled together. In some embodiments, the primary T cells are harvested from one or a plurality of individuals, and in some instances, the primary T cells or the pool of primary T cells are cultured in vitro. In some embodiments, the primary T cells or the pool of primary T cells are engineered to exogenously express CD47 and cultured in vitro.

In some embodiments, hypoimmunogenic T cells and non-activated T cells are propagated from a pool of primary T cells or progeny thereof, wherein the pool of primary T cells is isolated from one or more donor subjects different from the recipient patient, wherein the one or more donor subjects optionally comprise either one or more subjects that are RhD positive, one or more subjects that are RhD negative, or a mixture of subjects that are RhD positive and subjects that are RhD negative.

In some embodiments, hypoimmunogenic T cells and non-activated T cells are derived from a pool of iPSCs or progeny thereof, wherein the pool of iPSCs is derived from host cells isolated from one or more donor subjects different from the recipient patient, wherein the one or more donor subjects optionally comprise either one or more subjects that are RhD positive, one or more subjects that are RhD negative, or a mixture of subjects that are RhD positive and subjects that are RhD negative.

Exemplary primary T cells of the present disclosure are selected from the group consisting of cytotoxic T cells, helper T cells, memory T-cells, regulatory T cells, tissue infiltrating lymphocytes, and combinations thereof. In some embodiments, the primary T cells is a modified primary T cell. In some cases, the modified T cell comprise a modification causing the cell to express at least one chimeric antigen receptor that specifically binds to an antigen or epitope of interest expressed on the surface of at least one of a damaged cell, a dysplastic cell, an infected cell, an immunogenic cell, an inflamed cell, a malignant cell, a metaplastic cell, a mutant cell, and combinations thereof. In other cases, the modified T cell comprise a modification causing the cell to express at least one protein that modulates a biological effect of interest in an adjacent cell, tissue, or organ when the cell is in proximity to the adjacent cell, tissue, or organ. Useful modifications to primary T cells are described in detail in US2016/0348073 and WO2020/018620, the disclosures are incorporated herein in its entirety. Methods provided are useful for inactivation or ablation of MHC class I expression and/or MHC class II expression in cells such as but not limited to pluripotent stem cells and primary T cells. In some embodiments, genome editing technologies utilizing rare-cutting endonucleases (e.g., the CRISPR/Cas, TALEN, zinc finger nuclease, meganuclease, and homing endonuclease systems) are also used to reduce or eliminate expression of critical immune genes (e.g., by deleting genomic DNA of critical immune genes) in cells. In certain embodiments, genome editing technologies or other gene modulation technologies are used to insert tolerance-inducing factors in human cells, rendering them and the differentiated cells prepared therefrom hypoimmunogenic T cells. As such, the hypoimmunogenic T cells have reduced or eliminated expression of MHC I and MHC II expression. In some embodiments, the cells are nonimmunogenic (e.g., do not induce an immune response) in a recipient subject.

The genome editing techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

The practice of the some embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989): Maniatis et al., Molecular Cloning: A Laboratory Manual (1982): Ausubel et al., Current Protocols in Molecular Biology (John Wiley and Sons, updated July 2008): Short Protocols in Molecular Biology: A Compendium of Methods from Current Protocols in Molecular Biology, Greene Pub. Associates and Wiley-Interscience: Glover, DNA Cloning: A Practical Approach, vol. I & II (IRL Press, Oxford, 1985); Anand, Techniques for the Analysis of Complex Genomes, (Academic Press, New York, 1992): Transcription and Translation (B. Hames & S. Higgins, Eds., 1984); Perbal, A Practical Guide to Molecular Cloning (1984): Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991): Annual Review of Immunology: as well as monographs in journals such as Advances in Immunology.

Provided herein are cells comprising a modification of one or more targeted polynucleotide sequences that regulates the expression of RHD, MHC I and/or MHC II. In some embodiments, the cells comprise increased expression of CD47. In some embodiments, the cells comprise an exogenous or recombinant CD47 polypeptide. In some embodiments, the cell also includes a modification to increase expression of one selected from the group consisting of CD200, HLA-G, HLA-E, HLA-C, HLA-E heavy chain, PD-L1, IDO1, CTLA4-Ig. IL-10, IL-35, FASL, Serpinb9, CCl21, and Mfge8. In some embodiments, the cell further comprises a tolerogenic factor (e.g., an immunomodulatory molecule) selected from the group consisting of DUX4, CD200, HLA-G, HLA-E, HLA-C, HLA-E heavy chain, PD-L1, IDO1, CTLA4-Ig, IL-10, IL-35, FASL, Serpinb9), CCl21, and Mfge8.

In some embodiments, the cell comprises a genomic modification of one or more targeted polynucleotide sequences that regulates the expression of the RHD gene. In some embodiments, a genetic editing system is used to modify one or more targeted polynucleotide sequences. In some embodiments, the targeted polynucleotide sequence is RHD gene. In certain embodiments, the genome of the cell has been altered to reduce or delete critical components of RHD gene expression.

In many embodiments, the primary T cells or the pool of primary T cells are engineered to express one or more chimeric antigen receptors (CARs). The CARs can be any known to those skilled in the art. Useful CARs include those that bind an antigen selected from a group that includes CD19, CD20, CD22, CD38, CD123, CD138, and BCMA. In some cases, the CARs are the same or equivalent to those used in FDA-approved CAR-T cell therapies such as, but not limited to, those used in tisagenlecleucel and axicabtagene ciloleucel, or others under investigation in clinical trials.

In some embodiments, hypoimmunogenic T cells and non-activated T cells comprise a gene modification in the RHD gene. In some embodiments, the gene modification affects one allele of the RHD gene. In some embodiments, the gene modification affects two alleles of the RHD gene. In some embodiments, the gene modification is an insertion, deletion, or disruption of the RHD gene. In some embodiments, the gene modification is a homozygous modification of the RHD gene. In some embodiments, the gene modification is a heterozygous modification of the RHD gene. In some embodiments, RHD expression is interfered with by targeting the RHD locus (e.g., knocking out expression of RHD), or by targeting transcriptional regulators of RHD expression. In some embodiments, RHD is “knocked-out” of a cell. A cell that has a knocked-out RHD gene may exhibit reduced or eliminated expression of the knocked-out gene.

Gene editing using a rare-cutting endonuclease such as, but not limited to Cas9 or Cas12a is utilized to a targeted disruption of one or more genes encoding a histocompatibility determinant, such as but not limited to, an RHD gene.

In some instances, the targeted disruption of the RHD gene targets any one of its coding exons. In some embodiments, the entire coding sequence or a large portion thereof of the gene is disrupted or excised. In some embodiments, insertion-deletions (indel) by way of CRISPR/Cas editing are introduced into the cell to disruption of the RHD gene.

In some embodiments, an RNA guided-DNA nuclease is used to target the coding sequence of the RHD gene to introduce deleterious variations of the RHD gene and disruption of RhD function. In other embodiments, the untranslated region, intron sequence and/or exon sequences of the RHD gene are targeted.

In some embodiments, the deleterious variation of the RHD gene comprises an indel. In some embodiments, the deleterious variation of the RHD gene comprises a deletion. In some embodiments, the deleterious variation of the RHD gene comprises an insertion. In some embodiments, the deleterious variation of the RHD gene comprises a frameshift mutation. In some embodiments, the deleterious variation of the RHD gene comprises a substitution. In some embodiments, the deleterious variation of the RHD gene comprises a point mutation. In some embodiments, the deleterious variation of the RHD gene reduced the expression of the gene. In some embodiments, the deleterious variation of the RHD gene comprises a loss-of-function mutation.

In some embodiments, the hypoimmunogenic T cells and non-activated T cells are histocompatible cells. In some embodiments, the histocompatibility of the cells is determined using a complement mediated cell killing assay. A non-limiting example of such as assay is an XCelligence SP platform (ACEA BioSciences).

In some embodiments, the cell comprises a genomic modification of one or more targeted polynucleotide sequences that regulates the expression of MHC I and/or MHC II. In some embodiments, a genetic editing system is used to modify one or more targeted polynucleotide sequences. In some embodiments, the targeted polynucleotide sequence is one or more selected from the group consisting of B2M and CIITA. In some cases, the targeted polynucleotide sequence is NLRC5. In certain embodiments, the genome of the cell has been altered to reduce or delete critical components of HLA expression.

Reduction of MHC I and/or MHC II expression can be accomplished, for example, by one or more of the following: (1) targeting the polymorphic HLA alleles (HLA-A, HLA-B, HLA-C) and MHC-II genes directly: (2) removal of B2M, which will prevent surface trafficking of all MHC-I molecules; and/or (3) deletion of components of the MHC enhanceosomes, such as LRC5, RFX-5, RFXANK, RFXAP, IRF1, NF-Y (including NFY-A, NFY-B, NFY-C), and CIITA that are critical for HLA expression.

In certain embodiments, HLA expression is interfered with. In some embodiments, HLA expression is interfered with by targeting individual HLAs (e.g., knocking out expression of HLA-A, HLA-B and/or HLA-C), targeting transcriptional regulators of HLA expression (e.g., knocking out expression of NLRC5, CIITA, RFX5, RFXAP, RFXANK, NFY-A, NFY-B, NFY-C and/or IRF-1), blocking surface trafficking of MHC class I molecules (e.g., knocking out expression of B2M and/or TAP1), and/or targeting with HLA-Razor (see, e.g., WO2016183041).

In some embodiments, the cells disclosed herein do not express one or more human leukocyte antigens (e.g., HLA-A, HLA-B and/or HLA-C) corresponding to MHC-I and/or MHC-II and are thus characterized as being hypoimmunogenic. For example, in some embodiments, the cells disclosed herein have been modified such that the cell or a differentiated cell prepared therefrom do not express or exhibit reduced expression of one or more of the following MHC-I molecules: HLA-A, HLA-B and HLA-C. In some embodiments, one or more of HLA-A, HLA-B and HLA-C may be “knocked-out” of a cell. A cell that has a knocked-out HLA-A gene, HLA-B gene, and/or HLA-C gene may exhibit reduced or eliminated expression of each knocked-out gene.

In certain embodiments, gRNAs that allow simultaneous deletion of all MHC class I alleles by targeting a conserved region in the HLA genes are identified as HLA Razors. In some embodiments, the gRNAs are part of a CRISPR system. In some embodiments, the gRNAs are part of a TALEN system. In some embodiments, an HLA Razor targeting an identified conserved region in HLAs is described in WO2016183041. In some embodiments, multiple HLA Razors targeting identified conserved regions are utilized. It is generally understood that any guide that targets a conserved region in HLAs can act as an HLA Razor.

In some embodiments, the present disclosure provides a cell or population thereof comprising a genome in which a gene has been edited to delete a contiguous stretch of genomic DNA, thereby reducing or eliminating surface expression of MHC class I molecules in the cell or population thereof. In some embodiments, the present disclosure provides a cell or population thereof comprising a genome in which a gene has been edited to delete a contiguous stretch of genomic DNA, thereby reducing or eliminating surface expression of MHC class II molecules in the cell or population thereof. In some embodiments, the present disclosure provides a cell or population thereof comprising a genome in which one or more genes has been edited to delete a contiguous stretch of genomic DNA, thereby reducing or eliminating surface expression of MHC class I and II molecules in the cell or population thereof.

In certain embodiments, the expression of MHC I or MHC II is modulated by targeting and deleting a contiguous stretch of genomic DNA thereby reducing or eliminating expression of a target gene selected from the group consisting of B2M and CIITA. In other cases, the target gene is NLRC5.

In some embodiments, the cells and methods described herein include genomically editing human cells to cleave CIITA gene sequences as well as editing the genome of such cells to alter one or more additional target polynucleotide sequences such as, but not limited to, B2M and NLRC5. In some embodiments, the cells and methods described herein include genomically editing human cells to cleave B2M gene sequences as well as editing the genome of such cells to alter one or more additional target polynucleotide sequences such as, but not limited to, CIITA and NLRC5. In some embodiments, the cells and methods described herein include genomically editing human cells to cleave NLRC5 gene sequences as well as editing the genome of such cells to alter one or more additional target polynucleotide sequences such as, but not limited to, B2M and CIITA.

### B. Pharmaceutical Compositions

Provided herein are pharmaceutical compositions comprising one or more hypoimmunogenic T cell or non-activated T cell described herein, and a pharmaceutically acceptable additive, carrier, diluent or excipient. In some embodiments, the composition comprises one or more populations of cells selected from the group consisting of a population of hypoimmunogenic T cells, a population of non-activated T cells, a population hypoimmunogenic CD19 CAR T cells, and a population of hypoimmunogenic CD22 CAR T cells, a population of CD19/CD22 CAR T cells, and a pharmaceutically acceptable additive, carrier, diluent or excipient. In some embodiments, the composition comprises one or more populations of hypoimmunogenic T cells, and a pharmaceutically acceptable additive, carrier, diluent or excipient. In some embodiments, the composition comprises one or more populations of non-activated T cells, and a pharmaceutically acceptable additive, carrier, diluent or excipient. In some embodiments, the composition comprises one or more populations of hypoimmunogenic CD19 CAR T cells, and a pharmaceutically acceptable additive, carrier, diluent or excipient. In some embodiments, the composition comprises one or more populations of hypoimmunogenic CD22 CAR T cells, and a pharmaceutically acceptable additive, carrier, diluent or excipient. In some embodiments, the composition comprises one or more populations of hypoimmunogenic CD19 CAR T cells and one or more populations of hypoimmunogenic CD22 CAR T cells, and a pharmaceutically acceptable additive, carrier, diluent or excipient. In some embodiments, the composition comprises one or more populations of CD19/CD22 CAR T cells, and a pharmaceutically acceptable additive, carrier, diluent or excipient, wherein the CD19/CD22 CAR T cells comprise CD19 CARs and CD22 CARs. In some embodiments, the composition comprises one or more populations of CD19/CD22 CAR T cells, and a pharmaceutically acceptable additive, carrier, diluent or excipient, wherein the CD19/CD22 CAR T cells comprise CD19 CARs and CD22 CARs, wherein the CD19 CAR and the CD22 CAR are encoded by a single bicistronic polynucleotide. In some embodiments, the composition comprises one or more populations of CD19/CD22 CAR T cells, and a pharmaceutically acceptable additive, carrier, diluent or excipient, wherein the CD19/CD22 CAR T cells comprise CD19 CARs and CD22 CARS, wherein the CD19 CAR and the CD22 CAR are encoded by two separate polynucleotides. In some embodiments, the composition comprises one or more populations of CD19/CD22 CAR T cells, and a pharmaceutically acceptable additive, carrier, diluent or excipient, wherein the CD19/CD22 CAR T cells comprise CD19/CD22 bispecific CARs. In some embodiments, the composition comprises one or more populations of CD19/CD22 CAR T cells, and a pharmaceutically acceptable additive, carrier, diluent or excipient, wherein the CD19/CD22 CAR T cells comprise a CD19/CD22 bivalent CAR.

In some embodiments, the pharmaceutical composition provided herein further include a pharmaceutically acceptable carrier. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). In some embodiments, the pharmaceutical composition includes a pharmaceutically acceptable buffer (e.g., neutral buffer saline or phosphate buffered saline).

C. Therapeutic Cells Derived from T Cells

Provided herein are hypoimmunogenic T cells and non-activated T cells that evade immune recognition. In some embodiments, the hypoimmunogenic T and non-activated T cells are produced (e.g., generated, cultured, propagated, or derived) from T cells such as primary T cells. In some instances, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, the pool of T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of T cells does not include cells from the patient. In some embodiments, one or more of the donor subjects from which the pool of T cells is obtained are different from the patient. In some embodiments, the primary T cells are from a pool of primary T cells from one or more donor subjects that are different than the recipient subject (e.g., the patient administered the cells). The primary T cells can be obtained from 1, 2, 3, 4, 5, 6, 7, 8, 9, 10, 20, 50, 100 or more donor subjects and pooled together. The primary T cells can be obtained from 1 or more, 2 or more, 3 or more, 4 or more, 5 or more, 6 or more, 7 or more, 8 or more, 9 or more, 10, or more 20 or more, 50 or more, or 100 or more donor subjects and pooled together. In some embodiments, the primary T cells are harvested from one or a plurality of individuals, and in some instances, the primary T cells or the pool of primary T cells are cultured in vitro. In some embodiments, the primary T cells are harvested from one more donor subjects, wherein the one or more donor subjects optionally comprise either one or more subjects that are RhD positive, one or more subjects that are RhD negative, or a mixture of subjects that are RhD positive and subjects that are RhD negative. In some embodiments, primary T cells or a pool of primary T cells are engineered to exogenously express CD47 and cultured in vitro.

In some embodiments, the primary T cells include, but are not limited to, CD3+ T cells, CD4+ T cells, CD8+ T cells, naïve T cells, regulatory T (Treg) cells, non-regulatory T cells. Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells that express CD45RA (TEMRA cells), tissue-resident memory (Trm) cells, virtual memory T cells, innate memory T cells, memory stem cell (Tsc), γδ T cells, and any other subtype of T cells.

In some embodiments, the primary T cell and any cell propagated, derived, or differentiated from such a primary T cell is modified to exhibit reduced expression of RhD antigen. In some embodiments, the primary T cell and any cell differentiated from such a primary T cell is modified to exhibit reduced expression of MHC class I human leukocyte antigens. In other embodiments, the primary T cell and any cell differentiated from such a pluripotent stem cell is modified to exhibit reduced expression of MHC class II human leukocyte antigens. In some embodiments, the primary T cell and any cell differentiated from such a pluripotent stem cell is modified to exhibit reduced expression of RhD antigen and MHC class I and II human leukocyte antigens. In some embodiments, the primary T cell and any cell differentiated from such a pluripotent stem cell is modified to exhibit reduced expression of RhD antigen and MHC class I and/or II human leukocyte antigens and exhibit increased CD47 expression. In some instances, the cell overexpresses CD47 by harboring one or more CD47 transgenes.

In some embodiments, the cells used in the methods described herein evade immune recognition and responses when administered to a patient (e.g., recipient subject). The cells can evade killing by immune cells in vitro and in vivo. In some embodiments, the cells evade killing by macrophages and NK cells. In some embodiments, the cells are ignored by immune cells or a subject's immune system. In other words, the cells administered in accordance with the methods described herein are not detectable by immune cells of the immune system. In some embodiments, the cells are cloaked and therefore avoid immune rejection.

Methods of determining whether a hypoimmunogenic T cell or a non-activated T cell evades immune recognition include, but are not limited to, IFN-γ Elispot assays, microglia killing assays, cell engraftment animal models, cytokine release assays, ELISAs, killing assays using bioluminescence imaging or chromium release assay or Xcelligence analysis, mixed-lymphocyte reactions, immunofluorescence analysis, etc.

Therapeutic cells outlined herein are useful to treat a disorder such as, but not limited to, a cancer, a genetic disorder, a chronic infectious disease, an autoimmune disorder, a neurological disorder, and the like.

D. Therapeutic Cells Derived from Pluripotent Stem Cells

Provided herein are hypoimmunogenic T cells and non-activated T cells that evade immune recognition. In some embodiments, the hypoimmunogenic T cells and non-activated T cells are produced (e.g., generated, cultured, propagated, or derived) from hypoimmune induced pluripotent stem cells.

In some embodiments, the induced pluripotent stem cells are produced from a pool of host cells such that the host cells are from one or more subjects (e.g., one or more human including one or more healthy humans). In some embodiments, the pool of host cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of host cells does not include cells from the patient. In some embodiments, one or more of the donor subjects from which the pool of host cells is obtained are different from the patient. In some embodiments, the induced pluripotent stem cells are produced from a pool of primary host cells from one or more donor subjects that are different than the recipient subject (e.g., the patient administered the cells). The pool of host cells can be obtained from 1, 2, 3, 4, 5, 6, 7, 8, 9, 10, 20, 50, 100 or more donor subjects and pooled together. The pool of host cells can be obtained from 1 or more, 2 or more, 3 or more, 4 or more, 5 or more, 6, or more, 7 or more, 8 or more, 9 or more, 10, or more 20 or more, 50 or more, or 100 or more donor subjects and pooled together. In some embodiments, the pool of host cells is from one or a plurality of individuals. In some embodiments, the host cells are harvested from one more donor subjects, wherein the one or more donor subjects optionally comprise either one or more subjects that are RhD positive, one or more subjects that are RhD negative, or a mixture of subjects that are RhD positive and subjects that are RhD negative. In some embodiments, the induced pluripotent stem cells are engineered to exogenously express CD47 and cultured in vitro.

In some embodiments, the pluripotent stem cell and any cell differentiated from such a pluripotent stem cell is modified to exhibit reduced expression of RhD antigen. In some embodiments, the pluripotent stem cell and any cell differentiated from such a pluripotent stem cell is modified to exhibit reduced expression of MHC class I human leukocyte antigens. In other embodiments, the pluripotent stem cell and any cell differentiated from such a pluripotent stem cell is modified to exhibit reduced expression of MHC class II human leukocyte antigens. In some embodiments, the pluripotent stem cell and any cell differentiated from such a pluripotent stem cell is modified to exhibit reduced expression of RhD antigen and MHC class I and II human leukocyte antigens. In some embodiments, the pluripotent stem cell and any cell differentiated from such a pluripotent stem cell is modified to exhibit reduced expression of RhD antigen and MHC class I and/or II human leukocyte antigens and exhibit increased CD47 expression. In some instances, the cell overexpresses CD47 by harboring one or more CD47 transgenes.

In some embodiments, the cells used in the methods described herein evade immune recognition and responses when administered to a patient (e.g., recipient subject). The cells can evade killing by immune cells in vitro and in vivo. In some embodiments, the cells evade killing by macrophages and NK cells. In some embodiments, the cells are ignored by immune cells or a subject's immune system. In other words, the cells administered in accordance with the methods described herein are not detectable by immune cells of the immune system. In some embodiments, the cells are cloaked and therefore avoid immune rejection.

Methods of determining whether a pluripotent stem cell and any cell differentiated from such a pluripotent stem cell evades immune recognition include, but are not limited to, IFN-γ Elispot assays, microglia killing assays, cell engraftment animal models, cytokine release assays, ELISAs, killing assays using bioluminescence imaging or chromium release assay or Xcelligence analysis, mixed-lymphocyte reactions, immunofluorescence analysis, etc.

Therapeutic cells outlined herein are useful to treat a disorder such as, but not limited to, a cancer, a genetic disorder, a chronic infectious disease, an autoimmune disorder, a neurological disorder, and the like.

### E. CD47

In some embodiments, the present technology provides a cell or population thereof that has been modified to express the tolerogenic factor (e.g., immunomodulatory polypeptide) CD47. In some embodiments, the present disclosure provides a method for altering a cell genome to express CD47. In some embodiments, the stem cell expresses exogenous CD47. In some instances, the cell expresses an expression vector comprising a nucleotide sequence encoding a human CD47 polypeptide. In some instances, the cell expresses a nucleotide sequence encoding a human CD47 polypeptide such that the nucleotide sequence is inserted into at least one allele of a safe harbor locus. In some instances, the cell expresses a nucleotide sequence encoding a human CD47 polypeptide such that the nucleotide sequence is inserted into at least one allele of an RHD locus. In some instances, the cell expresses a nucleotide sequence encoding a human CD47 polypeptide such that the nucleotide sequence is inserted into at least one allele of an AAVS1 locus. In some instances, the cell expresses a nucleotide sequence encoding a human CD47 polypeptide such that the nucleotide sequence is inserted into at least one allele of an CCR5 locus. In some instances, the cell expresses a nucleotide sequence encoding a human CD47 polypeptide such that the nucleotide sequence is inserted into at least one allele of a safe harbor gene locus, such as, but not limited to, a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, an LRP1 (CD91) gene locus, a HMGB1 gene locus, an ABO gene locus, an RHD gene locus, a FUT1 locus, and a KDM5D gene locus. In some instances, the cell expresses a nucleotide sequence encoding a human CD47 polypeptide such that the nucleotide sequence is inserted into at least one allele of a TRAC locus.

CD47 is a leukocyte surface antigen and has a role in cell adhesion and modulation of integrins. It is expressed on the surface of a cell and signals to circulating macrophages not to eat the cell.

In some embodiments, the cell outlined herein comprises a nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a nucleotide sequence encoding a CD47 polypeptide having an amino acid sequence as set forth in NCBI Ref. Sequence Nos. NP_001768.1 and NP_942088.1. In some embodiments, the cell comprises a nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises a nucleotide sequence for CD47 as set forth in NCBI Ref. Sequence Nos. NM_001777.3 and NM_198793.2.

In some embodiments, the cell comprises a CD47 polypeptide having at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a CD47 polypeptide having an amino acid sequence as set forth in NCBI Ref. Sequence Nos. NP_001768.1 and NP_942088.1.

In some embodiments, a suitable gene editing system (e.g., CRISPR/Cas system or any of the gene editing systems described herein) is used to facilitate the insertion of a polynucleotide encoding CD47, into a genomic locus of the hypoimmunogenic T cell. In some cases, the polynucleotide encoding CD47 is inserted into a safe harbor locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding CD47 is inserted into a B2M gene locus, a CIITA gene locus, a TRAC gene locus, or a TRB gene locus. In some embodiments, the polynucleotide encoding CD47 is inserted into any one of the gene loci depicted in Table 5 provided herein. In certain embodiments, the polynucleotide encoding CD47 is operably linked to a promoter.

In another embodiment, CD47 protein expression is detected using a Western blot of cell lysates probed with antibodies against the CD47 protein. In another embodiment, reverse transcriptase polymerase chain reactions (RT-PCR) are used to confirm the presence of the exogenous CD47 mRNA.

### F. RHD

In certain embodiments, the present technology disclosed herein modulates (e.g., reduces or eliminates) the expression of RhD antigen by targeting and modulating (e.g., reducing or eliminating) expression of the RHD gene. In some embodiments, the modulation occurs using a CRISPR/Cas system. In some embodiments, the cell has a reduced ability to induce an immune response in a recipient subject.

In some embodiments, the target polynucleotide sequence of the present technology is a variant of RHD gene. In some embodiments, the target polynucleotide sequence is a homolog of RHD gene. In some embodiments, the target polynucleotide sequence is an ortholog of RHD gene.

In some embodiments, the cells described herein comprise gene modifications at the gene locus encoding the RhD antigen protein. In other words, the cells comprise a genetic modification at the RHD locus. In some instances, the nucleotide sequence encoding the RhD antigen protein is set forth in RefSeq. Nos. NM_001127691.2, NM_001282868.1, NM_001282869.1, NM_001282871.1, or NM_016124.4, or in Genbank No. L08429. in some instances, the RHD gene locus is described in NCBI Gene ID No. 6007. In certain cases, the amino acid sequence of RhD antigen protein is depicted as NCBI GenBank No. AAA02679.1. Additional descriptions of the RhD protein and gene locus can be found in Uniprot No. Q02161, HGNC Ref. No. 10009, and OMIM Ref. No. 111680.

In some embodiments, the hypoimmunogenic T cells and non-activated T cells outlined herein comprise a genetic modification targeting the RHD gene. In some embodiments, the genetic modification targeting the RHD gene is generated by gene editing the RHD gene using gene editing tools such as but not limited to CRISPR/Cas, TALE-nucleases, zinc finger nucleases, other viral based gene editing system, or RNA interference. In some embodiments, the gene editing targets the coding sequence of the RHD gene. In some instances, the cells do not generate a functional RHD gene product. In the absence of the RHD gene product, the cells completely lack an Rh blood group antigen.

In some embodiments, a Cas9 or a Cas12a editing system is used to target a sequence of the RHD gene to introduce an insertion or deletion into the gene to disrupt its function, and in some instances, to render it inactive. In some embodiments, a single guide RNA is used. In some embodiments, dual guide RNAs are used. In some embodiments, any one of the gRNA target sequences of Tables 1A-1D are used. In some instances, more than one gRNA target sequences of Tables 1A-1D are used for gene editing. In some embodiments, a Cas9 editing system includes a Cas9 protein or a fragment thereof, a tracrRNA and a crRNA. In some embodiments, a Cas12a editing system includes a Cas12a protein or a fragment thereof and a crRNA.

In some embodiments, a frame-shift insertion-deletion is introduced in any coding sequence of the gene. In some embodiments, a modification within the UTRs, introns, or exons of the gene is added to disrupt the function of the RHD gene. In some embodiments, CRISPR/Cas editing comprising any one or more of the gRNA target sequences of Tables 1A-1D are utilized.

In some embodiments, a modification is introduced into the RHD gene to inactivate the gene. In some embodiments, coding exons such as exon 1 or exon 2 of the RHD gene are targeted. In some embodiments, coding exon 4 of the RHD gene are targeted. In some embodiments, coding exon 5 of the RHD gene are targeted. In some embodiments, coding exon 6 of the RHD gene are targeted. In some embodiments, coding exon 7 of the RHD gene are targeted. In some embodiments, coding exon 8 of the RHD gene are targeted. In some instances, a deletion is produced using a Cas editing system and a guide RNA target sequence targeting a sequence at the 5′ of the RHD gene and a guide RNA target sequence to an exon such as but not limited to exon 8. In some embodiments, one gRNA target sequence is the RHD 5′ UTR guide 1 of Table 1A and one gRNA target sequence is the RHD exon 8 guide 1 of Table 1. In some embodiments, a cell described herein comprises a homozygous modification of the RHD gene, thereby inactivating the gene.

TABLE 1A
Exemplary RHD gRNA target sequences

| | Guide | | | | |
|---|---|---|---|---|---|
| SEQ ID | RNA | | | Se- | |
| NO: | name | Position | Strand | quence | PAM |
| SEQ ID | RHD | 25290638 | −1 | CACCGA | TGG |
| NO: 1 | gRNA 1 | | | CAAAGC | |
| | | | | ACTCAT | |
| | | | | GG | |
| SEQ ID | RHD | 25284571 | 1 | TGGCCA | TGG |
| NO: 2 | gRNA 2 | | | AGATCT | |
| | | | | GACCGT | |
| | | | | GA | |
| SEQ ID | RHD | 25307729 | 1 | GGAGGC | CGG |
| NO: 3 | Exon 8 | | | GCTGCG | |
| | guide 1 | | | GTTCCT | |
| | | | | AC | |
| SEQ ID | RHD | 25272403 | −1 | TGGTTG | TGG |
| NO: 4 | 5′ UTR | | | TGCTGG | |
| | guide 1 | | | CCTCTC | |
| | | | | TA | |

TABLE 1B
Exemplary RHD gRNA target sequences

| Position | Strand | Sequence | PAM | Exon |
|---|---|---|---|---|
| 25306721 | 1 | GATACCGTCGGAGCCGGCAA | TGG | 7 |
| 25306715 | 1 | GTGCTTGATACCGTCGGAGC | CGG | 7 |
| 25306709 | 1 | CTGCTGGTGCTTGATACCGT | CGG | 7 |
| 25307756 | 1 | CTGCGGTTCCTACCGGTTCT | TGG | 8 |
| 25284622 | −1 | GTCTCCGGAAACTCGAGGTG | AGG | 2 |
| 25301582 | −1 | ACGGCATTCTTCCTTTCGAT | TGG | 5 |
| 25307749 | 1 | GGAGGCGCTGCGGTTCCTAC | CGG | 8 |
| 25284627 | −1 | GCTGTGTCTCCGGAAACTCG | AGG | 2 |
| 25301628 | 1 | CTATGCTGTAGCAGTCAGCG | TGG | 5 |
| 25303438 | 1 | GCTGGGCTGATCTCCGTCGG | GGG | 6 |
| 25284629 | 1 | GCTTCCTCACCTCGAGTTTC | CGG | 2 |
| 25301033 | −1 | TCCTCCGTTCCCTCGGGTAG | AGG | 4 |
| 25306657 | 1 | GGGCTACAACTTCAGCTTGC | TGG | 7 |
| 25284606 | 1 | CGTGATGGCGGCCATTGGCT | TGG | 2 |
| 25301613 | −1 | GCTGACTGCTACAGCATAGT | AGG | 5 |
| 25303436 | 1 | TGGCTGGGCTGATCTCCGTC | GGG | 6 |
| 25301040 | 1 | AAAGCCTCTACCCGAGGGAA | CGG | 4 |
| 25301582 | 1 | TGCTGAGAAGTCCAATCGAA | AGG | 5 |
| 25306658 | 1 | GGCTACAACTTCAGCTTGCT | GGG | 7 |
| 25284641 | 1 | CGAGTTTCCGGAGACACAGC | TGG | 2 |

TABLE 1C
Exemplary RHD gRNA target sequences
to target coding exons

| Position | Strand | Sequence | PAM |
|---|---|---|---|
| 25272568 | −1 | GGCAGCGCCGGACAGACCGC | GGG |
| 25272569 | −1 | AGGCAGCGCCGGACAGACCG | CGG |
| 25272572 | 1 | CTAAGTACCCGCGGTCTGTC | CGG |
| 25272580 | −1 | CCCAGAGGGGCAGGCAGCGC | CGG |
| 25272589 | −1 | GTGTTAGGGCCCAGAGGGGC | AGG |
| 25272590 | 1 | TCCGGCGCTGCCTGCCCCTC | TGG |
| 25272591 | 1 | CCGGCGCTGCCTGCCCCTCT | GGG |
| 25272593 | −1 | TCCAGTGTTAGGGCCCAGAG | GGG |
| 25272594 | −1 | TTCCAGTGTTAGGGCCCAGA | GGG |
| 25272595 | −1 | CTTCCAGTGTTAGGGCCCAG | AGG |
| 25272603 | 1 | GCCCCTCTGGGCCCTAACAC | TGG |
| 25272603 | −1 | GAGAGCTGCTTCCAGTGTTA | GGG |
| 25272604 | −1 | TGAGAGCTGCTTCCAGTGTT | AGG |
| 25272631 | −1 | AGTGGGTAAAAAAATAGAAG | AGG |
| 25272648 | −1 | CTCTAAGGAAGCGTCATAGT | GGG |
| 25272649 | −1 | CCTCTAAGGAAGCGTCATAG | TGG |
| 25272660 | 1 | CCACTATGACGCTTCCTTAG | AGG |
| 25272663 | −1 | GAGCCCCTTTTGATCCTCTA | AGG |
| 25272669 | 1 | CGCTTCCTTAGAGGATCAAA | AGG |
| 25272670 | 1 | GCTTCCTTAGAGGATCAAAA | GGG |
| 25272671 | 1 | CTTCCTTAGAGGATCAAAAG | GGG |
| 25272678 | 1 | AGAGGATCAAAAGGGGCTCG | TGG |
| 25284583 | −1 | CCGCCATCACGGTCAGATCT | TGG |
| 25284591 | 1 | TGGCCAAGATCTGACCGTGA | TGG |
| 25284594 | 1 | CCAAGATCTGACCGTGATGG | CGG |
| 25284594 | −1 | CAAGCCAATGGCCGCCATCA | CGG |
| 25284601 | 1 | CTGACCGTGATGGCGGCCAT | TGG |
| 25284606 | 1 | CGTGATGGCGGCCATTGGCT | TGG |
| 25284606 | −1 | GGTGAGGAAGCCCAAGCCAA | TGG |
| 25284607 | 1 | GTGATGGCGGCCATTGGCTT | GGG |
| 25284622 | −1 | GTCTCCGGAAACTCGAGGTG | AGG |
| 25284627 | −1 | GCTGTGTCTCCGGAAACTCG | AGG |
| 25284629 | 1 | GCTTCCTCACCTCGAGTTTC | CGG |
| 25284637 | −1 | CACTGCTCCAGCTGTGTCTC | CGG |
| 25284641 | 1 | CGAGTTTCCGGAGACACAGC | TGG |
| 25284651 | 1 | GAGACACAGCTGGAGCAGTG | TGG |
| 25284663 | −1 | CGCCAGCATGAAGAGGTTGA | AGG |
| 25284670 | −1 | CACCAAGCGCCAGCATGAAG | AGG |
| 25284672 | 1 | GGCCTTCAACCTCTTCATGC | TGG |
| 25284679 | 1 | AACCTCTTCATGCTGGCGCT | TGG |
| 25284689 | 1 | TGCTGGCGCTTGGTGTGCAG | TGG |
| 25284690 | 1 | GCTGGCGCTTGGTGTGCAGT | GGG |
| 25284702 | 1 | TGTGCAGTGGGCAATCCTGC | TGG |
| 25284706 | 1 | CAGTGGGCAATCCTGCTGGA | CGG |
| 25284706 | −1 | GGCTCAGGAAGCCGTCCAGC | AGG |
| 25284721 | −1 | TCCCAGAAGGGAACTGGCTC | AGG |
| 25284727 | −1 | CCACCTTCCCAGAAGGGAAC | TGG |
| 25284730 | 1 | TTCCTGAGCCAGTTCCCTTC | TGG |
| 25284731 | 1 | TCCTGAGCCAGTTCCCTTCT | GGG |
| 25284733 | −1 | TGATGACCACCTTCCCAGAA | GGG |
| 25284734 | −1 | GTGATGACCACCTTCCCAGA | AGG |
| 25284735 | 1 | GAGCCAGTTCCCTTCTGGGA | AGG |
| 25284738 | 1 | CCAGTTCCCTTCTGGGAAGG | TGG |
| 25290658 | −1 | CACCGACAAAGCACTCATGG | TGG |
| 25290661 | −1 | CAGCACCGACAAAGCACTCA | TGG |
| 25290667 | 1 | GGCCACCATGAGTGCTTTGT | CGG |
| 25290682 | 1 | TTTGTCGGTGCTGATCTCAG | TGG |
| 25290694 | 1 | GATCTCAGTGGATGCTGTCT | TGG |
| 25290695 | 1 | ATCTCAGTGGATGCTGTCTT | GGG |
| 25290696 | 1 | TCTCAGTGGATGCTGTCTTG | GGG |
| 25290700 | 1 | AGTGGATGCTGTCTTGGGGA | AGG |
| 25290709 | 1 | TGTCTTGGGGAAGGTCAACT | TGG |
| 25290718 | 1 | GAAGGTCAACTTGGCGCAGT | TGG |
| 25290721 | 1 | GGTCAACTTGGCGCAGTTGG | TGG |
| 25290727 | 1 | CTTGGCGCAGTTGGTGGTGA | TGG |
| 25290733 | 1 | GCAGTTGGTGGTGATGGTGC | TGG |
| 25290736 | 1 | GTTGGTGGTGATGGTGCTGG | TGG |
| 25290739 | 1 | GGTGGTGATGGTGCTGGTGG | AGG |
| 25290752 | 1 | CTGGTGGAGGTGACAGCTTT | AGG |
| 25290762 | 1 | TGACAGCTTTAGGCAACCTG | AGG |
| 25290766 | 1 | AGCTTTAGGCAACCTGAGGA | TGG |
| 25290767 | −1 | TATTACTGATGACCATCCTC | AGG |
| 25300960 | −1 | AGATGTGCATCATGTTCATG | TGG |
| 25300993 | 1 | TACGTGTTCGCAGCCTATTT | TGG |
| 25300994 | 1 | ACGTGTTCGCAGCCTATTTT | GGG |
| 25300995 | −1 | GGCCACAGACAGCCCAAAAT | AGG |
| 25301004 | 1 | AGCCTATTTTGGGCTGTCTG | TGG |
| 25301009 | 1 | ATTTTGGGCTGTCTGTGGCC | TGG |
| 25301016 | −1 | TAGAGGCTTTGGCAGGCACC | AGG |
| 25301023 | −1 | CCTCGGGTAGAGGCTTTGGC | AGG |
| 25301027 | −1 | GTTCCCTCGGGTAGAGGCTT | TGG |
| 25301033 | −1 | TCCTCCGTTCCCTCGGGTAG | AGG |
| 25301034 | 1 | CCTGCCAAAGCCTCTACCCG | AGG |
| 25301035 | 1 | CTGCCAAAGCCTCTACCCGA | GGG |
| 25301039 | −1 | TCTTTATCCTCCGTTCCCTC | GGG |
| 25301040 | 1 | AAAGCCTCTACCCGAGGGAA | CGG |
| 25301040 | −1 | ATCTTTATCCTCCGTTCCCT | CGG |
| 25301043 | 1 | GCCTCTACCCGAGGGAACGG | AGG |
| 25301088 | 1 | ACCCAGTTTGTCTGCCATGC | TGG |
| 25301529 | −1 | CCAGAACATCCACAAGAAGA | GGG |
| 25301530 | −1 | GCCAGAACATCCACAAGAAG | AGG |
| 25301540 | 1 | CCCTCTTCTTGTGGATGTTC | TGG |
| 25301552 | −1 | AGCAGAGCAGAGTTGAAACT | TGG |
| 25301582 | 1 | TGCTGAGAAGTCCAATCGAA | AGG |
| 25301582 | −1 | ACGGCATTCTTCCTTTCGAT | TGG |
| 25301601 | −1 | AGCATAGTAGGTGTTGAACA | CGG |
| 25301613 | −1 | GCTGACTGCTACAGCATAGT | AGG |
| 25301628 | 1 | CTATGCTGTAGCAGTCAGCG | TGG |
| 25301644 | 1 | AGCGTGGTGACAGCCATCTC | AGG |
| 25301645 | 1 | GCGTGGTGACAGCCATCTCA | GGG |
| 25301646 | −1 | AGCCAAGGATGACCCTGAGA | TGG |
| 25301655 | 1 | AGCCATCTCAGGGTCATCCT | TGG |
| 25301661 | −1 | CTTCCCTTGGGGGTGAGCCA | AGG |
| 25301668 | 1 | TCATCCTTGGCTCACCCCCA | AGG |
| 25301669 | 1 | CATCCTTGGCTCACCCCCAA | GGG |
| 25303341 | 1 | TTATGTGCACAGTGCGGTGT | TGG |
| 25303345 | 1 | GTGCACAGTGCGGTGTTGGC | AGG |
| 25303348 | 1 | CACAGTGCGGTGTTGGCAGG | AGG |
| 25303353 | 1 | TGCGGTGTTGGCAGGAGGCG | TGG |
| 25303359 | 1 | GTTGGCAGGAGGCGTGGCTG | TGG |
| 25303360 | 1 | TTGGCAGGAGGCGTGGCTGT | GGG |
| 25303374 | −1 | AGAAGGGATCAGGTGACACG | AGG |
| 25303384 | −1 | CAAGCCACGGAGAAGGGATC | AGG |
| 25303390 | −1 | CCATGGCAAGCCACGGAGAA | GGG |
| 25303391 | 1 | GTCACCTGATCCCTTCTCCG | TGG |
| 25303391 | −1 | ACCATGGCAAGCCACGGAGA | AGG |
| 25303397 | −1 | CCCAGCACCATGGCAAGCCA | CGG |
| 25303401 | 1 | CCCTTCTCCGTGGCTTGCCA | TGG |
| 25303407 | 1 | TCCGTGGCTTGCCATGGTGC | TGG |
| 25303407 | −1 | AGCCACAAGACCCAGCACCA | TGG |
| 25303408 | 1 | CCGTGGCTTGCCATGGTGCT | GGG |
| 25303416 | 1 | TGCCATGGTGCTGGGTCTTG | TGG |
| 25303420 | 1 | ATGGTGCTGGGTCTTGTGGC | TGG |
| 25303421 | 1 | TGGTGCTGGGTCTTGTGGCT | GGG |
| 25303435 | 1 | GTGGCTGGGCTGATCTCCGT | CGG |
| 25303436 | 1 | TGGCTGGGCTGATCTCCGTC | GGG |
| 25303437 | 1 | GGCTGGGCTGATCTCCGTCG | GGG |
| 25303438 | 1 | GCTGGGCTGATCTCCGTCGG | GGG |
| 25303440 | −1 | CAGGTACTTGGCTCCCCCGA | CGG |
| 25306613 | 1 | GGGTGTTGTAACCGAGTGCT | GGG |
| 25306613 | −1 | TGTGGGGAATCCCCAGCACT | CGG |
| 25306614 | 1 | GGTGTTGTAACCGAGTGCTG | GGG |
| 25306629 | −1 | TAGCCCATGATGGAGCTGTG | GGG |
| 25306630 | −1 | GTAGCCCATGATGGAGCTGT | GGG |
| 25306631 | −1 | TGTAGCCCATGATGGAGCTG | TGG |
| 25306636 | 1 | GATTCCCCACAGCTCCATCA | TGG |
| 25306637 | 1 | ATTCCCCACAGCTCCATCAT | GGG |
| 25306639 | −1 | GCTGAAGTTGTAGCCCATGA | TGG |
| 25306657 | 1 | GGGCTACAACTTCAGCTTGC | TGG |
| 25306658 | 1 | GGCTACAACTTCAGCTTGCT | GGG |
| 25306667 | 1 | TTCAGCTTGCTGGGTCTGCT | TGG |
| 25306693 | 1 | GATCATCTACATTGTGCTGC | TGG |
| 25306709 | 1 | CTGCTGGTGCTTGATACCGT | CGG |
| 25306715 | 1 | GTGCTTGATACCGTCGGAGC | CGG |
| 25306721 | 1 | GATACCGTCGGAGCCGGCAA | TGG |
| 25307668 | −1 | CTTCAGCCATTTTTACAgcc | agg |
| 25307673 | 1 | ctggaacctggcTGTAAAAA | TGG |
| 25307683 | 1 | gcTGTAAAAATGGCTGAAGC | AGG |
| 25307691 | 1 | AATGGCTGAAGCAGGTGATG | AGG |
| 25307706 | 1 | TGATGAGGAGCTGATGCGTT | TGG |
| 25307728 | 1 | GACGTGTCTCAGAGAAATCA | TGG |
| 25307731 | 1 | GTGTCTCAGAGAAATCATGG | AGG |
| 25307739 | 1 | GAGAAATCATGGAGGCGCTG | CGG |
| 25307749 | 1 | GGAGGCGCTGCGGTTCCTAC | CGG |
| 25307753 | −1 | GAAGGCATCCAAGAACCGGT | AGG |
| 25307756 | 1 | CTGCGGTTCCTACCGGTTCT | TGG |
| 25307757 | −1 | TGTAGAAGGCATCCAAGAAC | CGG |
| 25307771 | −1 | GCTATGGTTGTCTCTGTAGA | AGG |
| 25307787 | −1 | ATCCCTATAATTTGGGGCTA | TGG |
| 25307793 | −1 | TATGTGATCCCTATAATTTG | GGG |
| 25307794 | −1 | ATATGTGATCCCTATAATTT | GGG |
| 25307795 | 1 | CAACCATAGCCCCAAATTAT | AGG |
| 25307795 | −1 | GATATGTGATCCCTATAATT | TGG |
| 25307796 | 1 | AACCATAGCCCCAAATTATA | GGG |
| 25307811 | 1 | TTATAGGGATCACATATCAG | TGG |
| 25317015 | −1 | CCCCAATGCTGAGGAGGACC | TGG |
| 25317021 | −1 | TGAGTTCCCCAATGCTGAGG | AGG |
| 25317024 | 1 | TTCCAGGTCCTCCTCAGCAT | TGG |
| 25317024 | −1 | AGCTGAGTTCCCCAATGCTG | AGG |
| 25317025 | 1 | TCCAGGTCCTCCTCAGCATT | GGG |
| 25317026 | 1 | CCAGGTCCTCCTCAGCATTG | GGG |
| 25317038 | 1 | CAGCATTGGGGAACTCAGCT | TGG |

TABLE 1D
RHD gRNA target sequences

| Position | Strand | Sequence | PAM |
|---|---|---|---|
| 25272403 | −1 | TGGTTGTGCTGGCCTCTCTA | TGG |
| 25272414 | −1 | GGCTGCAAGGCTGGTTGTGC | TGG |
| 25272423 | −1 | CTTATCTCAGGCTGCAAGGC | TGG |
| 25272427 | −1 | AGGCCTTATCTCAGGCTGCA | AGG |
| 25272435 | 1 | CAGCCTTGCAGCCTGAGATA | AGG |
| 25272435 | −1 | CCCGCCAAAGGCCTTATCTC | AGG |
| 25272442 | 1 | GCAGCCTGAGATAAGGCCTT | TGG |
| 25272445 | 1 | GCCTGAGATAAGGCCTTTGG | CGG |
| 25272446 | 1 | CCTGAGATAAGGCCTTTGGC | GGG |
| 25272447 | −1 | ATAGGGGAGACACCCGCCAA | AGG |
| 25272463 | −1 | AGGGCTTGAGGGAGCGATAG | GGG |
| 25272464 | −1 | GAGGGCTTGAGGGAGCGATA | GGG |
| 25272465 | −1 | TGAGGGCTTGAGGGAGCGAT | AGG |
| 25272474 | −1 | ACACCTACTTGAGGGCTTGA | GGG |
| 25272475 | −1 | AACACCTACTTGAGGGCTTG | AGG |
| 25272482 | 1 | GCTCCCTCAAGCCCTCAAGT | AGG |
| 25272482 | −1 | TCTCTCCAACACCTACTTGA | GGG |
| 25272483 | −1 | CTCTCTCCAACACCTACTTG | AGG |
| 25272488 | 1 | TCAAGCCCTCAAGTAGGTGT | TGG |
| 25272495 | 1 | CTCAAGTAGGTGTTGGAGAG | AGG |
| 25272496 | 1 | TCAAGTAGGTGTTGGAGAGA | GGG |
| 25272497 | 1 | CAAGTAGGTGTTGGAGAGAG | GGG |
| 25272507 | 1 | TTGGAGAGAGGGGTGATGCC | TGG |
| 25272513 | 1 | AGAGGGGTGATGCCTGGTGC | TGG |
| 25272514 | −1 | GCAGGGGTTCCACCAGCACC | AGG |
| 25272516 | 1 | GGGGTGATGCCTGGTGCTGG | TGG |
| 25272530 | −1 | CTGTGTCCGTCTCTGTGCAG | GGG |
| 25272531 | −1 | CCTGTGTCCGTCTCTGTGCA | GGG |
| 25272532 | −1 | TCCTGTGTCCGTCTCTGTGC | AGG |
| 25272535 | 1 | GTGGAACCCCTGCACAGAGA | CGG |
| 25272542 | 1 | CCCTGCACAGAGACGGACAC | AGG |
| 25272563 | 1 | GGATGAGCTCTAAGTACCCG | CGG |
| 25272568 | −1 | GGCAGCGCCGGACAGACCGC | GGG |
| 25272569 | −1 | AGGCAGCGCCGGACAGACCG | CGG |
| 25272572 | 1 | CTAAGTACCCGCGGTCTGTC | CGG |
| 25272580 | −1 | CCCAGAGGGGCAGGCAGCGC | CGG |
| 25272589 | −1 | GTGTTAGGGCCCAGAGGGGC | AGG |
| 25272590 | 1 | TCCGGCGCTGCCTGCCCCTC | TGG |
| 25272591 | 1 | CCGGCGCTGCCTGCCCCTCT | GGG |
| 25272593 | −1 | TCCAGTGTTAGGGCCCAGAG | GGG |
| 25272594 | −1 | TTCCAGTGTTAGGGCCCAGA | GGG |
| 25272595 | −1 | CTTCCAGTGTTAGGGCCCAG | AGG |
| 25272603 | 1 | GCCCCTCTGGGCCCTAACAC | TGG |
| 25272603 | −1 | GAGAGCTGCTTCCAGTGTTA | GGG |
| 25272604 | −1 | TGAGAGCTGCTTCCAGTGTT | AGG |
| 25272631 | −1 | AGTGGGTAAAAAAATAGAAG | AGG |
| 25272648 | −1 | CTCTAAGGAAGCGTCATAGT | GGG |
| 25272649 | −1 | CCTCTAAGGAAGCGTCATAG | TGG |
| 25272660 | 1 | CCACTATGACGCTTCCTTAG | AGG |
| 25272663 | −1 | GAGCCCCTTTTGATCCTCTA | AGG |
| 25272669 | 1 | CGCTTCCTTAGAGGATCAAA | AGG |
| 25272670 | 1 | GCTTCCTTAGAGGATCAAAA | GGG |
| 25272671 | 1 | CTTCCTTAGAGGATCAAAAG | GGG |
| 25272678 | 1 | AGAGGATCAAAAGGGGCTCG | TGG |
| 25272691 | 1 | GGGCTCGTGGCATCCTATCA | AGG |
| 25272693 | −1 | CAATGAACTCTCACCTTGAT | AGG |
| 25272705 | 1 | CTATCAAGGTGAGAGTTCAT | TGG |
| 25272713 | 1 | GTGAGAGTTCATTGGAAAAG | TGG |
| 25272720 | 1 | TTCATTGGAAAAGTGGTCAC | AGG |
| 25272733 | 1 | TGGTCACAGGAGCAAATAGC | AGG |
| 25272734 | 1 | GGTCACAGGAGCAAATAGCA | GGG |
| 25272735 | 1 | GTCACAGGAGCAAATAGCAG | GGG |
| 25272739 | 1 | CAGGAGCAAATAGCAGGGGC | AGG |
| 25272740 | 1 | AGGAGCAAATAGCAGGGGCA | GGG |
| 25272741 | 1 | GGAGCAAATAGCAGGGGCAG | GGG |
| 25272744 | 1 | GCAAATAGCAGGGGCAGGGG | CGG |
| 25272745 | 1 | CAAATAGCAGGGGCAGGGGC | GGG |
| 25272746 | 1 | AAATAGCAGGGGCAGGGGCG | GGG |
| 25272747 | 1 | AATAGCAGGGGCAGGGGCGG | GGG |
| 25272750 | 1 | AGCAGGGGCAGGGGCGGGGG | AGG |
| 25272757 | 1 | GCAGGGGGGGGGAGGCCTG | TGG |
| 25272762 | −1 | CTGTGCCCCTGGAGAACCAC | AGG |
| 25272766 | 1 | GGGGAGGCCTGTGGTTCTCC | AGG |
| 25272767 | 1 | GGGAGGCCTGTGGTTCTCCA | GGG |
| 25272768 | 1 | GGAGGCCTGTGGTTCTCCAG | GGG |
| 25272773 | −1 | GAAAGGAACATCTGTGCCCC | TGG |
| 25272790 | −1 | TTCCTTGGGATTTTGTAGAA | AGG |
| 25272799 | 1 | TTCCTTTCTACAAAATCCCA | AGG |
| 25272804 | −1 | ATGGGGGAATCTTTTTCCTT | GGG |
| 25272805 | −1 | GATGGGGGAATCTTTTTCCT | TGG |
| 25272820 | −1 | CAATCTACGGAAGAAGATGG | GGG |
| 25272821 | −1 | GCAATCTACGGAAGAAGATG | GGG |
| 25272822 | −1 | TGCAATCTACGGAAGAAGAT | GGG |
| 25272823 | −1 | GTGCAATCTACGGAAGAAGA | TGG |
| 25272833 | −1 | CTGAATTTCGGTGCAATCTA | CGG |
| 25272845 | −1 | TTACATTGTTGGCTGAATTT | CGG |
| 25272856 | −1 | TAAAGGAAAGCTTACATTGT | TGG |
| 25272873 | −1 | CATGCCCAGGCTGCTTCTAA | AGG |
| 25272879 | 1 | GCTTTCCTTTAGAAGCAGCC | TGG |
| 25272880 | 1 | CTTTCCTTTAGAAGCAGCCT | GGG |
| 25272886 | −1 | TCACAGAAGAGGGCATGCCC | AGG |
| 25272896 | −1 | AAGGCAGGCTTCACAGAAGA | GGG |
| 25272897 | −1 | CAAGGCAGGCTTCACAGAAG | AGG |
| 25272911 | −1 | CTGTGCTGAAAAATCAAGGC | AGG |
| 25272915 | −1 | CTCACTGTGCTGAAAAATCA | AGG |
| 25272929 | 1 | TGATTTTTCAGCACAGTGAG | AGG |
| 25272941 | 1 | ACAGTGAGAGGCATCCTCTT | TGG |
| 25272944 | −1 | GAATTTGAGGAACACCAAAG | AGG |
| 25272957 | −1 | CATTTGGTAGAGGGAATTTG | AGG |
| 25272966 | −1 | TATGAAGACCATTTGGTAGA | GGG |
| 25272967 | −1 | TTATGAAGACCATTTGGTAG | AGG |
| 25272969 | 1 | CTCAAATTCCCTCTACCAAA | TGG |
| 25272973 | −1 | AGAGAATTATGAAGACCATT | TGG |
| 25273008 | −1 | TGCCACTGAGGAGAGAGAAG | GGG |
| 25273009 | −1 | TTGCCACTGAGGAGAGAGAA | GGG |
| 25273010 | −1 | CTTGCCACTGAGGAGAGAGA | AGG |
| 25273017 | 1 | TTCCCCTTCTCTCTCCTCAG | TGG |
| 25273020 | −1 | aaaaaaaTTCCTTGCCACTG | AGG |
| 25273022 | 1 | CTTCTCTCTCCTCAGTGGCA | AGG |
| 25273048 | 1 | ttttttatttttatagattt | agg |
| 25273049 | 1 | tttttatttttatagattta | ggg |
| 25273050 | 1 | ttttatttttatagatttag | ggg |
| 25273093 | 1 | TGCAAGCAatttcatgttgt | tgg |
| 25273094 | 1 | GCAAGCAatttcatgttgtt | ggg |
| 25273101 | 1 | atttcatgttgttgggtttt | tgg |
| 25273121 | 1 | tggtttttgtttcctttttg | tgg |
| 25273122 | −1 | atgagcgagaggccacaaaa | agg |
| 25273133 | −1 | agaaataagaaatgagcgag | agg |
| 25273152 | 1 | tcatttcttatttctttttg | agg |
| 25273156 | 1 | ttcttatttctttttgaggc | agg |
| 25273157 | 1 | tcttatttctttttgaggca | ggg |
| 25273176 | 1 | agggtctcactctgttgccc | agg |
| 25273182 | −1 | atgccactgcacttcagcct | ggg |
| 25273183 | −1 | catgccactgcacttcagcc | tgg |
| 25273190 | 1 | ttgcccaggctgaagtgcag | tgg |
| 25273201 | 1 | gaagtgcagtggcatgatca | tgg |
| 25273223 | −1 | tgcttgagactaggaggtca | agg |
| 25273229 | −1 | gaagattgcttgagactagg | agg |
| 25273232 | −1 | tgggaagattgcttgagact | agg |
| 25273251 | −1 | gcttcttgggaggctgaggt | ggg |
| 25273252 | −1 | agcttcttgggaggctgagg | tgg |
| 25273255 | −1 | cccagcttcttgggaggctg | agg |
| 25273261 | −1 | tgtggtcccagcttcttggg | agg |
| 25273264 | −1 | tcctgtggtcccagcttctt | ggg |
| 25273265 | 1 | acctcagcctcccaagaagc | tgg |
| 25273265 | −1 | ctcctgtggtcccagcttct | tgg |
| 25273266 | 1 | cctcagcctcccaagaagct | ggg |
| 25273274 | 1 | tcccaagaagctgggaccac | agg |
| 25273277 | 1 | caagaagctgggaccacagg | agg |
| 25273278 | 1 | aagaagctgggaccacagga | ggg |
| 25273279 | −1 | ggcatggtggtgccctcctg | tgg |
| 25273292 | −1 | aaaaaattagccaggcatgg | tgg |
| 25273293 | 1 | caggagggcaccaccatgcc | tgg |
| 25273295 | −1 | aaaaaaaaattagccaggca | tgg |
| 25273300 | −1 | aaaaaaaaaaaaaattagcc | agg |
| 25273330 | 1 | ttttttttttggtagagatg | tgg |
| 25273331 | 1 | tttttttttggtagagatgt | ggg |
| 25273346 | −1 | agaccagtctgggaaacaca | ggg |
| 25273347 | −1 | gagaccagtctgggaaacac | agg |
| 25273354 | 1 | tctccctgtgtttcccagac | tgg |
| 25273356 | −1 | caggagtttgagaccagtct | ggg |
| 25273357 | −1 | ccaggagtttgagaccagtc | tgg |
| 25273368 | 1 | ccagactggtctcaaactcc | tgg |
| 25273375 | −1 | ctggaggatcgcttgtgtcc | agg |
| 25273391 | −1 | ctttgggagactgaggctgg | agg |
| 25273394 | −1 | gcactttgggagactgaggc | tgg |
| 25273398 | −1 | tccagcactttgggagactg | agg |
| 25273407 | −1 | gcctgtaattccagcacttt | ggg |
| 25273408 | 1 | gcctcagtctcccaaagtgc | tgg |
| 25273408 | −1 | cgcctgtaattccagcactt | tgg |
| 25273417 | 1 | tcccaaagtgctggaattac | agg |
| 25273443 | −1 | TAGATATGAGCAAGAGAgct | ggg |
| 25273444 | −1 | ATAGATATGAGCAAGAGAgc | tgg |
| 25273471 | 1 | TATCTATACTAGTTTTCTTT | TGG |
| 25273492 | −1 | tgggggtggggggtAGCAAC | AGG |
| 25273502 | −1 | tcggtgggggtgggggtggg | ggg |
| 25273503 | −1 | gtcggtgggggtgggggtgg | ggg |
| 25273504 | −1 | ggtcggtgggggtgggggtg | ggg |
| 25273505 | −1 | gggtcggtgggggtgggggt | ggg |
| 25273506 | −1 | ggggtcggtgggggtggggg | tgg |
| 25273509 | −1 | GCTggggtcggtgggggtgg | ggg |
| 25273510 | −1 | AGCTggggtcggtgggggtg | ggg |
| 25273511 | −1 | AAGCTggggtcggtgggggt | ggg |
| 25273512 | −1 | AAAGCTggggtcggtggggg | tgg |
| 25273515 | −1 | AAGAAAGCTggggtcggtgg | ggg |
| 25273516 | −1 | GAAGAAAGCTggggtcggtg | ggg |
| 25273517 | −1 | AGAAGAAAGCTggggtcggt | ggg |
| 25273518 | −1 | GAGAAGAAAGCTggggtcgg | tgg |
| 25273521 | −1 | AGTGAGAAGAAAGCTggggt | cgg |
| 25273525 | −1 | CCTAAGTGAGAAGAAAGCTg | ggg |
| 25273526 | −1 | CCCTAAGTGAGAAGAAAGCT | ggg |
| 25273527 | −1 | CCCCTAAGTGAGAAGAAAGC | Tgg |
| 25273536 | 1 | ccccAGCTTTCTTCTCACTT | AGG |
| 25273537 | 1 | cccAGCTTTCTTCTCACTTA | GGG |
| 25273538 | 1 | ccAGCTTTCTTCTCACTTAG | GGG |
| 25273542 | 1 | CTTTCTTCTCACTTAGGGGC | TGG |
| 25273543 | 1 | TTTCTTCTCACTTAGGGGCT | GGG |
| 25273578 | −1 | TCAGCCATACCTTCTGGTTC | TGG |
| 25273580 | 1 | TCTATAAATCCAGAACCAGA | AGG |
| 25273584 | −1 | TCCCCTTCAGCCATACCTTC | TGG |
| 25273585 | 1 | AAATCCAGAACCAGAAGGTA | TGG |
| 25273592 | 1 | GAACCAGAAGGTATGGCTGA | AGG |
| 25273593 | 1 | AACCAGAAGGTATGGCTGAA | GGG |
| 25273594 | 1 | ACCAGAAGGTATGGCTGAAG | GGG |
| 25273597 | 1 | AGAAGGTATGGCTGAAGGGG | AGG |
| 25273598 | 1 | GAAGGTATGGCTGAAGGGGA | GGG |
| 25273602 | 1 | GTATGGCTGAAGGGGAGGGT | AGG |
| 25273609 | 1 | TGAAGGGGAGGGTAGGATGA | TGG |
| 25273647 | −1 | CAGTTGTCTCATCACAGTCT | GGG |
| 25273648 | −1 | ACAGTTGTCTCATCACAGTC | TGG |
| 25273683 | 1 | AATAAGACAGATGTCCACAA | TGG |
| 25273686 | −1 | AAAGCAAAGTCACACCATTG | TGG |
| 25273724 | 1 | AAAATATTGAAATGAGTTTC | AGG |
| 25273735 | 1 | ATGAGTTTCAGGCATCTCAG | TGG |
| 25273736 | 1 | TGAGTTTCAGGCATCTCAGT | GGG |
| 25273744 | 1 | AGGCATCTCAGTGGGCTGAT | AGG |
| 25273762 | 1 | ATAGGTTGTTGATAATAGAC | AGG |
| 25273763 | 1 | TAGGTTGTTGATAATAGACA | GGG |
| 25273775 | −1 | CTCAGGGACATTCTTCAAGG | AGG |
| 25273778 | −1 | TGTCTCAGGGACATTCTTCA | AGG |
| 25273791 | −1 | CAAGCTTCAACTTTGTCTCA | GGG |
| 25273792 | −1 | TCAAGCTTCAACTTTGTCTC | AGG |
| 25273809 | 1 | ACAAAGTTGAAGCTTGAGCC | TGG |
| 25273816 | −1 | GAACAAGCAAGGACTCAACC | AGG |
| 25273827 | −1 | TATCAACCTAGGAACAAGCA | AGG |
| 25273832 | 1 | TTGAGTCCTTGCTTGTTCCT | AGG |
| 25273838 | −1 | CTAGCCGTTCATATCAACCT | AGG |
| 25273845 | 1 | TGTTCCTAGGTTGATATGAA | CGG |
| 25273857 | 1 | GATATGAACGGCTAGTTAAC | TGG |
| 25273879 | 1 | GAAGCAAAGAGAAGTCATCC | TGG |
| 25273880 | 1 | AAGCAAAGAGAAGTCATCCT | GGG |
| 25273881 | 1 | AGCAAAGAGAAGTCATCCTG | GGG |
| 25273882 | 1 | GCAAAGAGAAGTCATCCTGG | GGG |
| 25273886 | −1 | TTGTCACTGCCATGGCCCCC | AGG |
| 25273888 | 1 | AGAAGTCATCCTGGGGGCCA | TGG |
| 25273894 | −1 | AGTCCTACTTGTCACTGCCA | TGG |
| 25273902 | 1 | GGGCCATGGCAGTGACAAGT | AGG |
| 25273909 | 1 | GGCAGTGACAAGTAGGACTT | AGG |
| 25273910 | 1 | GCAGTGACAAGTAGGACTTA | GGG |
| 25273913 | 1 | GTGACAAGTAGGACTTAGGG | AGG |
| 25273914 | 1 | TGACAAGTAGGACTTAGGGA | GGG |
| 25273929 | −1 | CAGCACCTTAAATGGTATAA | GGG |
| 25273930 | −1 | CCAGCACCTTAAATGGTATA | AGG |
| 25273935 | 1 | GGAAGCCCTTATACCATTTA | AGG |
| 25273937 | −1 | CTCTGGGCCAGCACCTTAAA | TGG |
| 25273941 | 1 | CCTTATACCATTTAAGGTGC | TGG |
| 25273951 | 1 | TTTAAGGTGCTGGCCCAGAG | AGG |
| 25273953 | −1 | GTCACTGAAGGCTCCTCTCT | GGG |
| 25273954 | −1 | TGTCACTGAAGGCTCCTCTC | TGG |
| 25273965 | −1 | TCTTGTTTGTCTGTCACTGA | AGG |
| 25273981 | 1 | AGTGACAGACAAACAAGAGC | TGG |
| 25274035 | −1 | tggaatgcattgaattgtat | tgg |
| 25274055 | −1 | GTCATACATGGTTGAAtgaa | tgg |
| 25274067 | −1 | CCCACATTGGATGTCATACA | TGG |
| 25274077 | 1 | ACCATGTATGACATCCAATG | TGG |
| 25274078 | 1 | CCATGTATGACATCCAATGT | GGG |
| 25274080 | −1 | CATGAGTCTGGATCCCACAT | TGG |
| 25274092 | −1 | agctctaatCATCATGAGTC | TGG |
| 25274133 | 1 | atgagcacttactatgtacc | agg |
| 25274140 | −1 | aaagcatgtagaatagtgcc | tgg |
| 25274171 | −1 | acctcattgggttattgtga | ggg |
| 25274172 | −1 | cacctcattgggttattgtg | agg |
| 25274181 | 1 | accctcacaataacccaatg | agg |
| 25274183 | −1 | ataatagtacccacctcatt | ggg |
| 25274184 | 1 | ctcacaataacccaatgagg | tgg |
| 25274184 | −1 | cataatagtacccacctcat | tgg |
| 25274185 | 1 | tcacaataacccaatgaggt | ggg |
| 25274216 | 1 | tgatcttcgtttttcatatg | agg |
| 25274224 | 1 | gtttttcatatgaggaaact | agg |
| 25274231 | 1 | atatgaggaaactaggcata | tgg |
| 25274253 | 1 | gatgttgagtaatttgccca | cgg |
| 25274258 | −1 | attgctagctgagcgaccgt | ggg |
| 25274259 | −1 | tattgctagctgagcgaccg | tgg |
| 25274300 | 1 | gtatttaaatttagccaccc | tgg |
| 25274303 | −1 | taaggaaactaaatccaggg | tgg |
| 25274306 | −1 | gtgtaaggaaactaaatcca | ggg |
| 25274307 | −1 | agtgtaaggaaactaaatcc | agg |
| 25274321 | −1 | ATgcataatggttaagtgta | agg |
| 25274333 | −1 | AATGGGGCCATGATgcataa | tgg |
| 25274337 | 1 | cacttaaccattatgcATCA | TGG |
| 25274349 | −1 | CTCAAGCCCACTGTAAAATG | GGG |
| 25274350 | −1 | ACTCAAGCCCACTGTAAAAT | GGG |
| 25274351 | −1 | GACTCAAGCCCACTGTAAAA | TGG |
| 25274353 | 1 | ATCATGGCCCCATTTTACAG | TGG |
| 25274354 | 1 | TCATGGCCCCATTTTACAGT | GGG |
| 25274383 | 1 | TCTTTgtcatataacccagt | agg |
| 25274386 | −1 | atagtggctgctaacctact | ggg |
| 25274387 | −1 | aatagtggctgctaacctac | tgg |
| 25274402 | −1 | aatctacagggttggaatag | tgg |
| 25274410 | −1 | ctagagtcaatctacagggt | tgg |
| 25274414 | −1 | gaccctagagtcaatctaca | ggg |
| 25274415 | −1 | ggaccctagagtcaatctac | agg |
| 25274422 | 1 | caaccctgtagattgactct | agg |
| 25274423 | 1 | aaccctgtagattgactcta | ggg |
| 25274436 | −1 | cggtgcaggggtaaagaaca | tgg |
| 25274448 | −1 | ACGTTAgtagcacggtgcag | ggg |
| 25274449 | −1 | TACGTTAgtagcacggtgca | ggg |
| 25274450 | −1 | CTACGTTAgtagcacggtgc | agg |
| 25274456 | −1 | TTGTACCTACGTTAgtagca | cgg |
| 25274462 | 1 | ctgcaccgtgctacTAACGT | AGG |
| 25274484 | −1 | ccgtaTAAAGTGAGTTTCTG | AGG |
| 25274495 | 1 | CCTCAGAAACTCACTTTAta | cgg |
| 25274506 | 1 | CACTTTAtacggaagctcag | agg |
| 25274509 | 1 | TTTAtacggaagctcagagg | agg |
| 25274510 | 1 | TTAtacggaagctcagagga | ggg |
| 25274523 | 1 | cagaggagggtccacaaccc | agg |
| 25274523 | −1 | cgtctcccctgcctgggttg | tgg |
| 25274527 | 1 | ggagggtccacaacccaggc | agg |
| 25274528 | 1 | gagggtccacaacccaggca | ggg |
| 25274529 | 1 | agggtccacaacccaggcag | ggg |
| 25274529 | −1 | caccatcgtctcccctgcct | ggg |
| 25274530 | −1 | acaccatcgtctcccctgcc | tgg |
| 25274538 | 1 | aacccaggcaggggagacga | tgg |
| 25274545 | 1 | gcaggggagacgatggtgtc | agg |
| 25274546 | 1 | caggggagacgatggtgtca | ggg |
| 25274547 | 1 | aggggagacgatggtgtcag | ggg |
| 25274550 | 1 | ggagacgatggtgtcagggg | agg |
| 25274551 | 1 | gagacgatggtgtcagggga | ggg |
| 25274554 | 1 | acgatggtgtcaggggaggg | agg |
| 25274570 | 1 | agggaggtgactgcccagcc | agg |
| 25274572 | −1 | tgagccttcaagacctggct | ggg |
| 25274573 | −1 | ctgagccttcaagacctggc | tgg |
| 25274577 | −1 | cctactgagccttcaagacc | tgg |
| 25274579 | 1 | actgcccagccaggtcttga | agg |
| 25274588 | 1 | ccaggtcttgaaggctcagt | agg |
| 25274600 | 1 | ggctcagtaggaattacctg | tgg |
| 25274601 | 1 | gctcagtaggaattacctgt | ggg |
| 25274605 | −1 | atgaccctcctttgtcccac | agg |
| 25274608 | 1 | aggaattacctgtgggacaa | agg |
| 25274611 | 1 | aattacctgtgggacaaagg | agg |
| 25274612 | 1 | attacctgtgggacaaagga | ggg |
| 25274626 | 1 | aaaggagggtcatccaagtg | agg |
| 25274627 | 1 | aaggagggtcatccaagtga | ggg |
| 25274628 | −1 | gcacccactgtgccctcact | tgg |
| 25274635 | 1 | tcatccaagtgagggcacag | tgg |
| 25274636 | 1 | catccaagtgagggcacagt | ggg |
| 25274644 | 1 | tgagggcacagtgggtgcca | tgg |
| 25274650 | −1 | tctattgtgtgtgcacgcca | tgg |
| 25274676 | 1 | acaatagagcAGACTGAGCC | TGG |
| 25274677 | 1 | caatagagcAGACTGAGCCT | GGG |
| 25274683 | −1 | GGCAATGCAATGTTAAGCCC | AGG |
| 25274698 | 1 | GGCTTAACATTGCATTGCCC | TGG |
| 25274704 | −1 | GTTTCCCCTTTTAGGCTCCA | GGG |
| 25274705 | −1 | TGTTTCCCCTTTTAGGCTCC | AGG |
| 25274709 | 1 | GCATTGCCCTGGAGCCTAAA | AGG |
| 25274710 | 1 | CATTGCCCTGGAGCCTAAAA | GGG |
| 25274711 | 1 | ATTGCCCTGGAGCCTAAAAG | GGG |
| 25274712 | −1 | ggccCTTTGTTTCCCCTTTT | AGG |
| 25274720 | 1 | GAGCCTAAAAGGGGAAACAA | AGg |
| 25274721 | 1 | AGCCTAAAAGGGGAAACAAA | Ggg |
| 25274725 | 1 | TAAAAGGGGAAACAAAGggc | cgg |
| 25274726 | 1 | AAAAGGGGAAACAAAGggcc | ggg |
| 25274733 | −1 | caggcgtgagccacgtcgcc | cgg |
| 25274734 | 1 | AAACAAAGggccgggcgacg | tgg |
| 25274752 | −1 | tcccaatgtgccgggattac | agg |
| 25274753 | 1 | gtggctcacgcctgtaatcc | cgg |
| 25274760 | −1 | ccttggcctcccaatgtgcc | ggg |
| 25274761 | 1 | cgcctgtaatcccggcacat | tgg |
| 25274761 | −1 | gccttggcctcccaatgtgc | cgg |
| 25274762 | 1 | gcctgtaatcccggcacatt | ggg |
| 25274765 | 1 | tgtaatcccggcacattggg | agg |
| 25274771 | 1 | cccggcacattgggaggcca | agg |
| 25274775 | 1 | gcacattgggaggccaaggc | tgg |
| 25274777 | −1 | ctcaggtgattctccagcct | tgg |
| 25274789 | 1 | caaggctggagaatcacctg | agg |
| 25274794 | 1 | ctggagaatcacctgaggtt | agg |
| 25274794 | −1 | ggtctcgaactcctaacctc | agg |
| 25274812 | 1 | ttaggagttcgagaccagcc | tgg |
| 25274815 | −1 | ttttgccatgttggccaggc | tgg |
| 25274819 | −1 | gcggttttgccatgttggcc | agg |
| 25274821 | 1 | cgagaccagcctggccaaca | tgg |
| 25274824 | −1 | gagatgcggttttgccatgt | tgg |
| 25274838 | −1 | ttataattttagtagagatg | cgg |
| 25274856 | 1 | tctactaaaattataaaaac | tgg |
| 25274860 | 1 | ctaaaattataaaaactggc | tgg |
| 25274861 | 1 | taaaattataaaaactggct | ggg |
| 25274866 | 1 | ttataaaaactggctgggtg | tgg |
| 25274869 | 1 | taaaaactggctgggtgtgg | tgg |
| 25274895 | −1 | taatggcctcccaagtagct | cgg |
| 25274896 | 1 | cgtctataatccgagctact | tgg |
| 25274897 | 1 | gtctataatccgagctactt | ggg |
| 25274900 | 1 | tataatccgagctacttggg | agg |
| 25274912 | −1 | gcgcccaggctggagtgtaa | tgg |
| 25274919 | 1 | gaggccattacactccagcc | tgg |
| 25274920 | 1 | aggccattacactccagcct | ggg |
| 25274922 | −1 | tctcactctggcgcccaggc | tgg |
| 25274926 | −1 | gaagtctcactctggcgccc | agg |
| 25274934 | −1 | tttgagatgaagtctcactc | tgg |
| 25274960 | −1 | ttgttgttgtttttgttgtt | tgg |
| 25274993 | 1 | agaacaacaaaaaaacaaaG | AGG |
| 25275001 | 1 | aaaaaaacaaaGAGGAGAGC | AGG |
| 25275002 | 1 | aaaaaacaaaGAGGAGAGCA | GGg |
| 25275007 | 1 | acaaaGAGGAGAGCAGGgac | tgg |
| 25275008 | 1 | caaaGAGGAGAGCAGGgact | ggg |
| 25275013 | 1 | AGGAGAGCAGGgactgggtg | tgg |
| 25275034 | −1 | cccaaagtgtttgggattac | agg |
| 25275042 | −1 | cttggtctcccaaagtgttt | ggg |
| 25275043 | −1 | ccttggtctcccaaagtgtt | tgg |
| 25275044 | 1 | gcctgtaatcccaaacactt | tgg |
| 25275045 | 1 | cctgtaatcccaaacacttt | ggg |
| 25275054 | 1 | ccaaacactttgggagacca | agg |
| 25275058 | 1 | acactttgggagaccaaggc | agg |
| 25275060 | −1 | ctcaggtgatctgcctgcct | tgg |
| 25275072 | 1 | caaggcaggcagatcacctg | agg |
| 25275077 | 1 | caggcagatcacctgaggtc | agg |
| 25275077 | −1 | ggtctcgaactcctgacctc | agg |
| 25275095 | 1 | tcaggagttcgagaccagcc | tgg |
| 25275098 | −1 | ttttaccatgttggccaggc | tgg |
| 25275102 | −1 | agggttttaccatgttggcc | agg |
| 25275104 | 1 | cgagaccagcctggccaaca | tgg |
| 25275107 | −1 | gagacagggttttaccatgt | tgg |
| 25275121 | −1 | ttgtatttttagtagagaca | ggg |
| 25275122 | −1 | tttgtatttttagtagagac | agg |
| 25275143 | 1 | ctaaaaatacaaaaattagc | cgg |
| 25275149 | 1 | atacaaaaattagccggatg | tgg |
| 25275151 | −1 | caggcacgtgccaccacatc | cgg |
| 25275152 | 1 | caaaaattagccggatgtgg | tgg |
| 25275170 | −1 | tcccaagcagctgggactac | agg |
| 25275178 | −1 | cctcagcttcccaagcagct | ggg |
| 25275179 | 1 | tgcctgtagtcccagctgct | tgg |
| 25275179 | −1 | ccctcagcttcccaagcagc | tgg |
| 25275180 | 1 | gcctgtagtcccagctgctt | ggg |
| 25275189 | 1 | cccagctgcttgggaagctg | agg |
| 25275190 | 1 | ccagctgcttgggaagctga | ggg |
| 25275193 | 1 | gctgcttgggaagctgaggg | agg |
| 25275212 | 1 | gaggagaattgcttgaaccc | agg |
| 25275215 | 1 | gagaattgcttgaacccagg | agg |
| 25275218 | −1 | ctcagcaacctctgcctcct | ggg |
| 25275219 | −1 | gctcagcaacctctgcctcc | tgg |
| 25275221 | 1 | tgcttgaacccaggaggcag | agg |
| 25275252 | −1 | tcacccagggtggagtgcag | tgg |
| 25275259 | 1 | catgccactgcactccaccc | tgg |
| 25275260 | 1 | atgccactgcactccaccct | ggg |
| 25275262 | −1 | tcccactctgtcacccaggg | tgg |
| 25275265 | −1 | gagtcccactctgtcaccca | ggg |
| 25275266 | −1 | agagtcccactctgtcaccc | agg |
| 25275271 | 1 | ctccaccctgggtgacagag | tgg |
| 25275272 | 1 | tccaccctgggtgacagagt | ggg |
| 25275316 | 1 | agtaataaataaaaataaaG | AGG |
| 25275317 | 1 | gtaataaataaaaataaaGA | GGG |
| 25275326 | 1 | aaaaataaaGAGGGAAGCAG | CGG |
| 25275327 | 1 | aaaataaaGAGGGAAGCAGC | GGG |
| 25275330 | 1 | ataaaGAGGGAAGCAGCGGG | TGG |
| 25275342 | 1 | GCAGCGGGTGGCAGACTCAC | TGG |
| 25275343 | 1 | CAGCGGGTGGCAGACTCACT | GGG |
| 25275360 | 1 | ACTGGGCTGCATACGAAGTT | TGG |
| 25275373 | 1 | CGAAGTTTGGCTTCAGTCTG | AGG |
| 25275386 | −1 | TCTCGCTGCTGTTTACTATT | CGG |
| 25275406 | 1 | AAACAGCAGCGAGACAAGTT | TGG |
| 25275407 | 1 | AACAGCAGCGAGACAAGTTT | GGG |
| 25275412 | 1 | CAGCGAGACAAGTTTGGGTT | TGG |
| 25275413 | 1 | AGCGAGACAAGTTTGGGTTT | GGG |
| 25275419 | 1 | ACAAGTTTGGGTTTGGGTCA | TGG |
| 25275422 | 1 | AGTTTGGGTTTGGGTCATGG | AGG |
| 25275435 | 1 | GTCATGGAGGAAGCCATGCC | AGG |
| 25275436 | 1 | TCATGGAGGAAGCCATGCCA | GGG |
| 25275437 | −1 | GCCCAACACCAGCCCTGGCA | TGG |
| 25275440 | 1 | GGAGGAAGCCATGCCAGGGC | TGG |
| 25275442 | −1 | CCTGTGCCCAACACCAGCCC | TGG |
| 25275446 | 1 | AGCCATGCCAGGGCTGGTGT | TGG |
| 25275447 | 1 | GCCATGCCAGGGCTGGTGTT | GGG |
| 25275453 | 1 | CCAGGGCTGGTGTTGGGCAC | AGG |
| 25275454 | 1 | CAGGGCTGGTGTTGGGCACA | GGG |
| 25275459 | 1 | CTGGTGTTGGGCACAGGGAA | AGG |
| 25275460 | 1 | TGGTGTTGGGCACAGGGAAA | GGG |
| 25275461 | 1 | GGTGTTGGGCACAGGGAAAG | GGG |
| 25275466 | 1 | TGGGCACAGGGAAAGGGGCA | TGG |
| 25275487 | −1 | CTACAGCCTCCACGCTGGTC | TGG |
| 25275489 | 1 | CTTGAGACACCAGACCAGCG | TGG |
| 25275492 | 1 | GAGACACCAGACCAGCGTGG | AGG |
| 25275492 | −1 | CTACACTACAGCCTCCACGC | TGG |
| 25275516 | 1 | TGTAGTGTAGTATTGACCTG | AGG |
| 25275521 | −1 | ATCAGAATGTTGAAGTCCTC | AGG |
| 25275534 | 1 | TGAGGACTTCAACATTCTGA | TGG |
| 25275566 | 1 | GATTttttgagcatgtacca | tgg |
| 25275572 | −1 | taaagtgtaatatataacca | tgg |
| 25275649 | 1 | acaataaatacatacaaatt | agg |
| 25275707 | 1 | tttcaaatTACTAATCATAA | TGG |
| 25275721 | 1 | TCATAATGGTGTCAATCTCC | AGG |
| 25275725 | 1 | AATGGTGTCAATCTCCAGGC | AGG |
| 25275726 | 1 | ATGGTGTCAATCTCCAGGCA | GGG |
| 25275728 | −1 | CTGTAGCAATGGACCCTGCC | TGG |
| 25275739 | −1 | actatcgtcaacTGTAGCAA | TGG |
| 25275752 | 1 | ATTGCTACAgttgacgatag | tgg |
| 25275777 | −1 | aaattatcaagaagactctg | agg |
| 25275869 | 1 | tgtgactgacagcttgtacg | agg |
| 25275896 | −1 | tcaagtgaacaaaagggaaa | agg |
| 25275902 | −1 | tggcagtcaagtgaacaaaa | ggg |
| 25275903 | −1 | atggcagtcaagtgaacaaa | agg |
| 25275922 | −1 | gattggaagcatagaaataa | tgg |
| 25275939 | −1 | tcgtgcagaaaaacacagat | tgg |
| 25275955 | 1 | ctgtgtttttctgcacgagt | tgg |
| 25275972 | −1 | actttcacaaaatgaagtaa | tgg |
| 25276002 | 1 | aagtttgttgagttaaactt | agg |
| 25276031 | −1 | caggactgaattcaattaag | tgg |
| 25276043 | 1 | cacttaattgaattcagtcc | tgg |
| 25276050 | −1 | atAatctattatagtttacc | agg |
| 25276078 | −1 | aatgtctttttagaattggc | agg |
| 25276082 | −1 | tcaaaatgtctttttagaat | tgg |
| 25276103 | 1 | aaagacattttgagacaatc | agg |
| 25276142 | 1 | tgaatatcttacgatataca | agg |
| 25276163 | 1 | ggattattgttaattttgtt | agg |
| 25276179 | 1 | tgttaggtatgataaaagca | tgg |
| 25276182 | 1 | taggtatgataaaagcatgg | tgg |
| 25276183 | 1 | aggtatgataaaagcatggt | ggg |
| 25276222 | −1 | caatgtgcctctctaacaga | tgg |
| 25276226 | 1 | taagtctccatctgttagag | agg |
| 25276239 | 1 | gttagagaggcacattgaaa | tgg |
| 25276251 | 1 | cattgaaatggcatgatatc | tgg |
| 25276252 | 1 | attgaaatggcatgatatct | ggg |
| 25276253 | 1 | ttgaaatggcatgatatctg | ggg |
| 25276277 | −1 | tctgtactttttcttttttc | tgg |
| 25276291 | 1 | gaaaaaagaaaaagtacaga | agg |
| 25276310 | 1 | aaggattatagaaacaagat | tgg |
| 25276337 | 1 | atgtgacaatcatcagagtt | tgg |
| 25276343 | 1 | caatcatcagagtttggaga | tgg |
| 25276344 | 1 | aatcatcagagtttggagat | ggg |
| 25276352 | 1 | gagtttggagatgggcacgt | agg |
| 25276353 | 1 | agtttggagatgggcacgta | ggg |
| 25276434 | 1 | aaaaaaaaaaaaaaaCACCC | TGG |
| 25276440 | −1 | cctccctaaatgctCAGCCA | GGG |
| 25276441 | −1 | gcctccctaaatgctCAGCC | AGG |
| 25276447 | 1 | aaCACCCTGGCTGagcattt | agg |
| 25276448 | 1 | aCACCCTGGCTGagcattta | ggg |
| 25276451 | 1 | CCCTGGCTGagcatttaggg | agg |
| 25276459 | 1 | Gagcatttagggaggccaag | tgg |
| 25276460 | 1 | agcatttagggaggccaagt | ggg |
| 25276461 | 1 | gcatttagggaggccaagtg | ggg |
| 25276463 | −1 | tttaagcgatcctccccact | tgg |
| 25276464 | 1 | tttagggaggccaagtgggg | agg |
| 25276480 | 1 | ggggaggatcgcttaaacca | agg |
| 25276486 | −1 | taggctcgtcttgaactcct | tgg |
| 25276499 | 1 | aaggagttcaagacgagcct | agg |
| 25276505 | −1 | ggggtctccctatgtttcct | agg |
| 25276508 | 1 | aagacgagcctaggaaacat | agg |
| 25276509 | 1 | agacgagcctaggaaacata | ggg |
| 25276524 | −1 | ttttttttagagatgggggg | ggg |
| 25276525 | −1 | tttttttttagagatggggg | ggg |
| 25276526 | −1 | ttttttttttagagatgggg | ggg |
| 25276527 | −1 | tttttttttttagagatggg | ggg |
| 25276528 | −1 | ttttttttttttagagatgg | ggg |
| 25276529 | −1 | tttttttttttttagagatg | ggg |
| 25276530 | −1 | ttttttttttttttagagat | ggg |
| 25276531 | −1 | tttttttttttttttagaga | tgg |
| 25276573 | 1 | ctttaaaatttaacccagtg | tgg |
| 25276575 | −1 | taggcatgtgccaccacact | ggg |
| 25276576 | 1 | taaaatttaacccagtgtgg | tgg |
| 25276576 | −1 | ataggcatgtgccaccacac | tgg |
| 25276594 | −1 | tactgagtagctgggactat | agg |
| 25276602 | −1 | cctcagcctactgagtagct | ggg |
| 25276603 | −1 | acctcagcctactgagtagc | tgg |
| 25276607 | 1 | tatagtcccagctactcagt | agg |
| 25276613 | 1 | cccagctactcagtaggctg | agg |
| 25276620 | 1 | actcagtaggctgaggtgag | agg |
| 25276635 | 1 | gtgagaggcttgcttgagcc | tgg |
| 25276636 | 1 | tgagaggcttgcttgagcct | ggg |
| 25276642 | −1 | cactgcagcctcaagctccc | agg |
| 25276645 | 1 | tgcttgagcctgggagcttg | agg |
| 25276654 | 1 | ctgggagcttgaggctgcag | tgg |
| 25276655 | 1 | tgggagcttgaggctgcagt | ggg |
| 25276659 | 1 | agcttgaggctgcagtggga | cgg |
| 25276660 | 1 | gcttgaggctgcagtgggac | ggg |
| 25276678 | −1 | tcgcccatgctggagtgaag | tgg |
| 25276685 | 1 | tgtaccacttcactccagca | tgg |
| 25276686 | 1 | gtaccacttcactccagcat | ggg |
| 25276688 | −1 | tcttgctctgtcgcccatgc | tgg |
| 25276711 | −1 | tttttattttttttgagaca | ggg |
| 25276712 | −1 | Atttttattttttttgagac | agg |
| 25276731 | 1 | aaaaaaaataaaaaTATTTG | AGG |
| 25276741 | 1 | aaaaTATTTGAGGTGAAGCG | AGG |
| 25276781 | 1 | AAAATATAAATAAAACATAA | Agg |
| 25276785 | 1 | TATAAATAAAACATAAAggc | tgg |
| 25276786 | 1 | ATAAATAAAACATAAAggct | ggg |
| 25276794 | 1 | AACATAAAggctgggtgtag | tgg |
| 25276812 | −1 | tcccaaagtgctgggattac | agg |
| 25276820 | −1 | ctttggcctcccaaagtgct | ggg |
| 25276821 | 1 | cgcctgtaatcccagcactt | tgg |
| 25276821 | −1 | gctttggcctcccaaagtgc | tgg |
| 25276822 | 1 | gcctgtaatcccagcacttt | ggg |
| 25276825 | 1 | tgtaatcccagcactttggg | agg |
| 25276835 | 1 | gcactttgggaggccaaagc | agg |
| 25276837 | −1 | acctcgtgatctgcctgctt | tgg |
| 25276847 | 1 | gccaaagcaggcagatcacg | agg |
| 25276852 | 1 | agcaggcagatcacgaggtc | tgg |
| 25276858 | 1 | cagatcacgaggtctggaga | tgg |
| 25276870 | 1 | tctggagatggagaccatcc | tgg |
| 25276873 | −1 | tttcatcgtgttagccagga | tgg |
| 25276877 | −1 | ggggtttcatcgtgttagcc | agg |
| 25276896 | −1 | ttgtatttttggtagagatg | ggg |
| 25276897 | −1 | tttgtatttttggtagagat | ggg |
| 25276898 | −1 | ttttgtatttttggtagaga | tgg |
| 25276907 | −1 | ggctaatttttttgtatttt | tgg |
| 25276920 | 1 | aaaaatacaaaaaaattagc | cgg |
| 25276921 | 1 | aaaatacaaaaaaattagcc | ggg |
| 25276926 | 1 | acaaaaaaattagccgggtg | tgg |
| 25276928 | −1 | caggcacccgccaccacacc | cgg |
| 25276929 | 1 | aaaaaattagccgggtgtgg | tgg |
| 25276932 | 1 | aaattagccgggtgtggtgg | cgg |
| 25276933 | 1 | aattagccgggtgtggtggc | ggg |
| 25276947 | −1 | tcccaagtagctgggactac | agg |
| 25276955 | −1 | cctcagcctcccaagtagct | ggg |
| 25276956 | 1 | tgcctgtagtcccagctact | tgg |
| 25276956 | −1 | gcctcagcctcccaagtagc | tgg |
| 25276957 | 1 | gcctgtagtcccagctactt | ggg |
| 25276960 | 1 | tgtagtcccagctacttggg | agg |
| 25276966 | 1 | cccagctacttgggaggctg | agg |
| 25276970 | 1 | gctacttgggaggctgaggc | agg |
| 25276977 | 1 | gggaggctgaggcaggagaa | tgg |
| 25276989 | 1 | caggagaatggcgtgaaccc | agg |
| 25276992 | 1 | gagaatggcgtgaacccagg | agg |
| 25276995 | 1 | aatggcgtgaacccaggagg | cgg |
| 25276995 | −1 | cactgaaagctccgcctcct | ggg |
| 25276996 | −1 | tcactgaaagctccgcctcc | tgg |
| 25277031 | −1 | ttgcccaggctggagtgcag | tgg |
| 25277038 | 1 | tacgccactgcactccagcc | tgg |
| 25277039 | 1 | acgccactgcactccagcct | ggg |
| 25277041 | −1 | tctcgctctgttgcccaggc | tgg |
| 25277045 | −1 | ggagtctcgctctgttgccc | agg |
| 25277066 | −1 | tattttcatttttttttaga | cgg |
| 25277109 | −1 | TCATATTGCAACTAATGGCA | GGG |
| 25277110 | −1 | TTCATATTGCAACTAATGGC | AGG |
| 25277114 | −1 | ATTCTTCATATTGCAACTAA | TGG |
| 25277158 | 1 | GCATATCAAATCCTTCTCAT | TGG |
| 25277158 | −1 | GGAATATTGGTCCAATGAGA | AGG |
| 25277171 | −1 | AAGGTGCCCTAAGGGAATAT | TGG |
| 25277175 | 1 | CATTGGACCAATATTCCCTT | AGG |
| 25277176 | 1 | ATTGGACCAATATTCCCTTA | GGG |
| 25277179 | −1 | AGCTTTGGAAGGTGCCCTAA | GGG |
| 25277180 | −1 | TAGCTTTGGAAGGTGCCCTA | AGG |
| 25277190 | −1 | TTGAGTCTCCTAGCTTTGGA | AGG |
| 25277193 | 1 | TTAGGGCACCTTCCAAAGCT | AGG |
| 25277194 | −1 | AGCCTTGAGTCTCCTAGCTT | TGG |
| 25277203 | 1 | TTCCAAAGCTAGGAGACTCA | AGG |
| 25277226 | −1 | AAGCCACCCCTCACTTGCTC | AGG |
| 25277229 | 1 | TATGACATCCTGAGCAAGTG | AGG |
| 25277230 | 1 | ATGACATCCTGAGCAAGTGA | GGG |
| 25277231 | 1 | TGACATCCTGAGCAAGTGAG | GGG |
| 25277234 | 1 | CATCCTGAGCAAGTGAGGGG | TGG |
| 25277241 | 1 | AGCAAGTGAGGGGTGGCTTC | TGG |
| 25277242 | 1 | GCAAGTGAGGGGTGGCTTCT | GGG |
| 25277301 | −1 | CTAGGCTATTCTATCTCTAA | AGG |
| 25277319 | −1 | actttgagaaacaTGGATCT | AGG |
| 25277326 | −1 | ggaccacactttgagaaaca | TGG |
| 25277334 | 1 | GATCCAtgtttctcaaagtg | tgg |
| 25277347 | −1 | atgctgaggcagcaggtctg | ggg |
| 25277348 | −1 | gatgctgaggcagcaggtct | ggg |
| 25277349 | −1 | agatgctgaggcagcaggtc | tgg |
| 25277354 | −1 | ccaggagatgctgaggcagc | agg |
| 25277361 | −1 | taaatttccaggagatgctg | agg |
| 25277365 | 1 | cctgctgcctcagcatctcc | tgg |
| 25277372 | −1 | tgcatttctactaaatttcc | agg |
| 25277405 | −1 | tgatcagtaggtctggccta | ggg |
| 25277406 | −1 | ctgatcagtaggtctggcct | agg |
| 25277412 | −1 | gagcttctgatcagtaggtc | tgg |
| 25277417 | −1 | gcccagagcttctgatcagt | agg |
| 25277426 | 1 | gacctactgatcagaagctc | tgg |
| 25277427 | 1 | acctactgatcagaagctct | ggg |
| 25277432 | 1 | ctgatcagaagctctgggcc | tgg |
| 25277433 | 1 | tgatcagaagctctgggcct | ggg |
| 25277434 | 1 | gatcagaagctctgggcctg | ggg |
| 25277439 | −1 | aacacagactgctgggcccc | agg |
| 25277446 | −1 | ttgtgaaaacacagactgct | ggg |
| 25277447 | −1 | cttgtgaaaacacagactgc | tgg |
| 25277467 | 1 | tgtgttttcacaagccctct | tgg |
| 25277470 | −1 | gcacagaagaatcaccaaga | ggg |
| 25277471 | −1 | tgcacagaagaatcaccaag | agg |
| 25277503 | 1 | catgaaagttcgagaattcc | tgg |
| 25277510 | −1 | atttgaatcagtctagctcc | agg |
| 25277537 | −1 | ccaaggtctctaagatacag | agg |
| 25277548 | 1 | cctctgtatcttagagacct | tgg |
| 25277549 | 1 | ctctgtatcttagagacctt | ggg |
| 25277554 | −1 | gaggttgactaatctgccca | agg |
| 25277573 | −1 | gtagaaacagaggcagaaag | agg |
| 25277583 | −1 | tctgacagaagtagaaacag | agg |
| 25277596 | 1 | tctgtttctacttctgtcag | agg |
| 25277630 | 1 | tgtttcattaagttgttgaa | agg |
| 25277717 | 1 | gagttttgctcttattgccc | agg |
| 25277718 | 1 | agttttgctcttattgccca | ggg |
| 25277719 | 1 | gttttgctcttattgcccag | ggg |
| 25277723 | −1 | tcgcaccactgcactcccct | ggg |
| 25277724 | −1 | atcgcaccactgcactcccc | tgg |
| 25277729 | 1 | tattgcccaggggagtgcag | tgg |
| 25277740 | 1 | ggagtgcagtggtgcgatct | tgg |
| 25277756 | −1 | aacctgggaggtggaggttg | cgg |
| 25277762 | −1 | tacttgaacctgggaggtgg | agg |
| 25277765 | 1 | caccgcaacctccacctccc | agg |
| 25277765 | −1 | aattacttgaacctgggagg | tgg |
| 25277768 | −1 | gagaattacttgaacctggg | agg |
| 25277771 | −1 | caggagaattacttgaacct | ggg |
| 25277772 | −1 | gcaggagaattacttgaacc | tgg |
| 25277790 | −1 | gctactcgggaggctgaggc | agg |
| 25277794 | −1 | cccagctactcgggaggctg | agg |
| 25277800 | −1 | tgtaatcccagctactcggg | agg |
| 25277803 | −1 | gcctgtaatcccagctactc | ggg |
| 25277804 | 1 | gcctcagcctcccgagtagc | tgg |
| 25277804 | −1 | tgcctgtaatcccagctact | cgg |
| 25277805 | 1 | cctcagcctcccgagtagct | ggg |
| 25277813 | 1 | tcccgagtagctgggattac | agg |
| 25277831 | −1 | acaaaattagccgggcgtgg | tgg |
| 25277832 | 1 | caggcatgcgccaccacgcc | cgg |
| 25277834 | −1 | aatacaaaattagccgggcg | tgg |
| 25277839 | −1 | ctaaaaatacaaaattagcc | ggg |
| 25277840 | −1 | actaaaaatacaaaattagc | cgg |
| 25277859 | 1 | ttttgtatttttagtagaga | tgg |
| 25277860 | 1 | tttgtatttttagtagagat | ggg |
| 25277861 | 1 | ttgtatttttagtagagatg | ggg |
| 25277875 | 1 | gagatggggtttctccatgt | tgg |
| 25277878 | −1 | cgagaccagcctcaccaaca | tgg |
| 25277880 | 1 | ggggtttctccatgttggtg | agg |
| 25277884 | 1 | tttctccatgttggtgaggc | tgg |
| 25277905 | 1 | ggtctcgaactcccaacctc | agg |
| 25277905 | −1 | cgggtgcatcacctgaggtt | ggg |
| 25277906 | −1 | gcgggtgcatcacctgaggt | tgg |
| 25277910 | −1 | caaggcgggtgcatcacctg | agg |
| 25277922 | 1 | ctcaggtgatgcacccgcct | tgg |
| 25277924 | −1 | gcactttgggaggccaaggc | ggg |
| 25277925 | −1 | agcactttgggaggccaagg | cgg |
| 25277928 | −1 | cccagcactttgggaggcca | agg |
| 25277934 | −1 | tgtaatcccagcactttggg | agg |
| 25277937 | −1 | gcctgtaatcccagcacttt | ggg |
| 25277938 | 1 | gccttggcctcccaaagtgc | tgg |
| 25277938 | −1 | cgcctgtaatcccagcactt | tgg |
| 25277939 | 1 | ccttggcctcccaaagtgct | ggg |
| 25277947 | 1 | tcccaaagtgctgggattac | agg |
| 25277965 | −1 | agctttTGggccaggcgcgg | tgg |
| 25277966 | 1 | caggcgtgagccaccgcgcc | tgg |
| 25277968 | −1 | taaagctttTGggccaggcg | cgg |
| 25277973 | −1 | gaaattaaagctttTGggcc | agg |
| 25277978 | −1 | attaagaaattaaagctttT | Ggg |
| 25277979 | −1 | aattaagaaattaaagcttt | TGg |
| 25278043 | −1 | aatacaatcaccagggtagc | tgg |
| 25278044 | 1 | ttgttttcttccagctaccc | tgg |
| 25278050 | −1 | aatgctcaatacaatcacca | ggg |
| 25278051 | −1 | aaatgctcaatacaatcacc | agg |
| 25278067 | 1 | tgattgtattgagcattttc | tgg |
| 25278068 | 1 | gattgtattgagcattttct | ggg |
| 25278069 | 1 | attgtattgagcattttctg | ggg |
| 25278098 | 1 | ttctttgctgtaatgactac | tgG |
| 25278103 | 1 | tgctgtaatgactactgGTC | TGG |
| 25278119 | −1 | tgcccatctggtcTCATCAC | AGG |
| 25278127 | 1 | TGACCTGTGATGAgaccaga | tgg |
| 25278128 | 1 | GACCTGTGATGAgaccagat | ggg |
| 25278131 | −1 | ctccactgcccctgcccatc | tgg |
| 25278132 | 1 | TGTGATGAgaccagatgggc | agg |
| 25278133 | 1 | GTGATGAgaccagatgggca | ggg |
| 25278134 | 1 | TGATGAgaccagatgggcag | ggg |
| 25278140 | 1 | gaccagatgggcaggggcag | tgg |
| 25278143 | 1 | cagatgggcaggggcagtgg | agg |
| 25278163 | 1 | aggagattctagagatattt | agg |
| 25278196 | 1 | gctgtacttgatgaaaagag | tgg |
| 25278197 | 1 | ctgtacttgatgaaaagagt | ggg |
| 25278198 | 1 | tgtacttgatgaaaagagtg | ggg |
| 25278206 | 1 | atgaaaagagtggggagtta | agg |
| 25278210 | 1 | aaagagtggggagttaaggc | tgg |
| 25278229 | 1 | ctggctgcagatgtatgatt | tgg |
| 25278239 | 1 | atgtatgatttggcatagag | agg |
| 25278253 | −1 | ctgtctctcatctcaggaac | tgg |
| 25278259 | −1 | ccccttctgtctctcatctc | agg |
| 25278268 | 1 | ttcctgagatgagagacaga | agg |
| 25278269 | 1 | tcctgagatgagagacagaa | ggg |
| 25278270 | 1 | cctgagatgagagacagaag | ggg |
| 25278273 | 1 | gagatgagagacagaagggg | agg |
| 25278274 | 1 | agatgagagacagaagggga | ggg |
| 25278279 | 1 | agagacagaaggggagggac | agg |
| 25278287 | 1 | aaggggagggacaggttgtg | agg |
| 25278316 | 1 | gaacaatgatatgttcattc | tgg |
| 25278317 | 1 | aacaatgatatgttcattct | ggg |
| 25278322 | 1 | tgatatgttcattctgggct | tgg |
| 25278330 | 1 | tcattctgggcttggagtta | agg |
| 25278331 | 1 | cattctgggcttggagttaa | ggg |
| 25278332 | 1 | attctgggcttggagttaag | ggg |
| 25278344 | −1 | GCTTCCCCTAAGCatatcat | agg |
| 25278349 | 1 | aaggggcctatgatatGCTT | AGG |
| 25278350 | 1 | aggggcctatgatatGCTTA | GGG |
| 25278351 | 1 | ggggcctatgatatGCTTAG | GGG |
| 25278382 | −1 | ggtggctgttatgcagcaat | agg |
| 25278400 | −1 | ttaagccactaagtttgggg | tgg |
| 25278403 | −1 | attttaagccactaagtttg | ggg |
| 25278404 | −1 | tattttaagccactaagttt | ggg |
| 25278405 | −1 | ctattttaagccactaagtt | tgg |
| 25278406 | 1 | aacagccaccccaaacttag | tgg |
| 25278431 | −1 | atgatcatgagtaaattaaa | agg |
| 25278452 | 1 | tactcatgatcatgattctg | tgg |
| 25278464 | 1 | tgattctgtggtgcaacaac | tgg |
| 25278465 | 1 | gattctgtggtgcaacaact | ggg |
| 25278469 | 1 | ctgtggtgcaacaactgggc | tgg |
| 25278470 | 1 | tgtggtgcaacaactgggct | ggg |
| 25278479 | 1 | acaactgggctgggttcagc | tgg |
| 25278480 | 1 | caactgggctgggttcagct | ggg |
| 25278506 | 1 | ttcttctgttagtttcaccc | agg |
| 25278507 | 1 | tcttctgttagtttcaccca | ggg |
| 25278512 | −1 | gcagatgcatgaatgaccct | ggg |
| 25278513 | −1 | tgcagatgcatgaatgaccc | tgg |
| 25278530 | 1 | tcattcatgcatctgcagtt | tgg |
| 25278531 | 1 | cattcatgcatctgcagttt | ggg |
| 25278532 | 1 | attcatgcatctgcagtttg | ggg |
| 25278535 | 1 | catgcatctgcagtttgggg | tgg |
| 25278536 | 1 | atgcatctgcagtttggggt | ggg |
| 25278540 | 1 | atctgcagtttggggtggga | tgg |
| 25278552 | −1 | cacgtgaatgaggtcatctg | agg |
| 25278562 | −1 | AACTgccaaacacgtgaatg | agg |
| 25278568 | 1 | gatgacctcattcacgtgtt | tgg |
| 25278575 | 1 | tcattcacgtgtttggcAGT | TGG |
| 25278586 | 1 | tttggcAGTTGGTGATTCAC | TGG |
| 25278587 | 1 | ttggcAGTTGGTGATTCACT | GGG |
| 25278588 | 1 | tggcAGTTGGTGATTCACTG | GGG |
| 25278589 | 1 | ggcAGTTGGTGATTCACTGG | GGG |
| 25278601 | −1 | GTAGGCGATTGTTACAGTAA | TGG |
| 25278616 | 1 | TACTGTAACAATCGCCTACC | AGG |
| 25278619 | −1 | TTAGGGAAGCTCTGCCTGGT | AGG |
| 25278623 | −1 | AGCCTTAGGGAAGCTCTGCC | TGG |
| 25278632 | 1 | TACCAGGCAGAGCTTCCCTA | AGG |
| 25278636 | −1 | CTCCTAGTTTGGAAGCCTTA | GGG |
| 25278637 | −1 | TCTCCTAGTTTGGAAGCCTT | AGG |
| 25278645 | 1 | TTCCCTAAGGCTTCCAAACT | AGG |
| 25278647 | −1 | CCCAGGATAGTCTCCTAGTT | TGG |
| 25278657 | 1 | TCCAAACTAGGAGACTATCC | TGG |
| 25278658 | 1 | CCAAACTAGGAGACTATCCT | GGG |
| 25278664 | −1 | GTATCCACAGCACAGGACCC | AGG |
| 25278671 | 1 | CTATCCTGGGTCCTGTGCTG | TGG |
| 25278671 | −1 | CTGAGTGGTATCCACAGCAC | AGG |
| 25278686 | −1 | GGTGGGGATGGGGGACTGAG | TGG |
| 25278695 | −1 | GGAATATGGGGTGGGGATGG | GGG |
| 25278696 | −1 | AGGAATATGGGGTGGGGATG | GGG |
| 25278697 | −1 | GAGGAATATGGGGTGGGGAT | GGG |
| 25278698 | −1 | TGAGGAATATGGGGTGGGGA | TGG |
| 25278702 | −1 | CCTTTGAGGAATATGGGGTG | GGG |
| 25278703 | −1 | GCCTTTGAGGAATATGGGGT | GGG |
| 25278704 | −1 | TGCCTTTGAGGAATATGGGG | TGG |
| 25278707 | −1 | CTCTGCCTTTGAGGAATATG | GGG |
| 25278708 | −1 | TCTCTGCCTTTGAGGAATAT | GGG |
| 25278709 | −1 | CTCTCTGCCTTTGAGGAATA | TGG |
| 25278713 | 1 | CCCCACCCCATATTCCTCAA | AGG |
| 25278716 | −1 | AGCCCCTCTCTCTGCCTTTG | AGG |
| 25278723 | 1 | TATTCCTCAAAGGCAGAGAG | AGG |
| 25278724 | 1 | ATTCCTCAAAGGCAGAGAGA | GGG |
| 25278725 | 1 | TTCCTCAAAGGCAGAGAGAG | GGG |
| 25278742 | 1 | GAGGGGCTACTAGAAGACAG | AGG |
| 25278760 | −1 | TGGAGTGTTTACATGTCACT | GGG |
| 25278761 | −1 | TTGGAGTGTTTACATGTCAC | TGG |
| 25278779 | 1 | CATGTAAACACTCCAAACCC | TGG |
| 25278780 | −1 | GTGTGGAAGGTGCCAGGGTT | TGG |
| 25278785 | −1 | CTGCAGTGTGGAAGGTGCCA | GGG |
| 25278786 | −1 | GCTGCAGTGTGGAAGGTGCC | AGG |
| 25278793 | −1 | GACCAAAGCTGCAGTGTGGA | AGG |
| 25278797 | −1 | GGCAGACCAAAGCTGCAGTG | TGG |
| 25278802 | 1 | CACCTTCCACACTGCAGCTT | TGG |
| 25278815 | 1 | GCAGCTTTGGTCTGCCCCTT | TGG |
| 25278816 | 1 | CAGCTTTGGTCTGCCCCTTT | GGG |
| 25278818 | −1 | AAAACAGAGATTTCCCAAAG | GGG |
| 25278819 | −1 | AAAAACAGAGATTTCCCAAA | GGG |
| 25278820 | −1 | GAAAAACAGAGATTTCCCAA | AGG |
| 25278839 | 1 | AAATCTCTGTTTTTCTTCCC | AGG |
| 25278845 | −1 | TCTCACCCCTCCAGCAGCCT | GGG |
| 25278846 | 1 | TGTTTTTCTTCCCAGGCTGC | TGG |
| 25278846 | −1 | CTCTCACCCCTCCAGCAGCC | TGG |
| 25278849 | 1 | TTTTCTTCCCAGGCTGCTGG | AGG |
| 25278850 | 1 | TTTCTTCCCAGGCTGCTGGA | GGG |
| 25278851 | 1 | TTCTTCCCAGGCTGCTGGAG | GGG |
| 25278864 | 1 | GCTGGAGGGGTGAGAGTCGC | CGG |
| 25278872 | −1 | GCCCACAGCCTCTACTCTAC | CGG |
| 25278875 | 1 | GAGAGTCGCCGGTAGAGTAG | AGG |
| 25278881 | 1 | CGCCGGTAGAGTAGAGGCTG | TGG |
| 25278882 | 1 | GCCGGTAGAGTAGAGGCTGT | GGG |
| 25278887 | 1 | TAGAGTAGAGGCTGTGGGCG | AGG |
| 25278890 | 1 | AGTAGAGGCTGTGGGCGAGG | AGG |
| 25278893 | 1 | AGAGGCTGTGGGCGAGGAGG | TGG |
| 25278896 | 1 | GGCTGTGGGCGAGGAGGTGG | CGG |
| 25278906 | 1 | GAGGAGGTGGCGGCCTCCTG | AGG |
| 25278908 | −1 | AAGACCACTGCAGCCTCAGG | AGG |
| 25278911 | −1 | GGAAAGACCACTGCAGCCTC | AGG |
| 25278915 | 1 | GCGGCCTCCTGAGGCTGCAG | TGG |
| 25278925 | 1 | GAGGCTGCAGTGGTCTTTCC | AGG |
| 25278932 | −1 | CCTGTGCTCCCACTGCTGCC | TGG |
| 25278934 | 1 | GTGGTCTTTCCAGGCAGCAG | TGG |
| 25278935 | 1 | TGGTCTTTCCAGGCAGCAGT | GGG |
| 25278943 | 1 | CCAGGCAGCAGTGGGAGCAC | AGG |
| 25278944 | 1 | CAGGCAGCAGTGGGAGCACA | GGG |
| 25278947 | 1 | GCAGCAGTGGGAGCACAGGG | TGG |
| 25278950 | 1 | GCAGTGGGAGCACAGGGTGG | AGG |
| 25278966 | −1 | CTTCACTCTCCCAGGCTCTA | GGG |
| 25278967 | 1 | TGGAGGTCAACCCTAGAGCC | TGG |
| 25278967 | −1 | GCTTCACTCTCCCAGGCTCT | AGG |
| 25278968 | 1 | GGAGGTCAACCCTAGAGCCT | GGG |
| 25278974 | −1 | ACACCCAGCTTCACTCTCCC | AGG |
| 25278981 | 1 | AGAGCCTGGGAGAGTGAAGC | TGG |
| 25278982 | 1 | GAGCCTGGGAGAGTGAAGCT | GGG |
| 25279002 | 1 | GGGTGTGACTTCAGAGCTGT | TGG |
| 25279020 | 1 | GTTGGTGCTGAAGTTTCTGC | AGG |
| 25279028 | 1 | TGAAGTTTCTGCAGGCCAGA | AGG |
| 25279031 | 1 | AGTTTCTGCAGGCCAGAAGG | AGG |
| 25279032 | 1 | GTTTCTGCAGGCCAGAAGGA | GGG |
| 25279032 | −1 | CCCACTCTTGCCCCTCCTTC | TGG |
| 25279033 | 1 | TTTCTGCAGGCCAGAAGGAG | GGG |
| 25279042 | 1 | GCCAGAAGGAGGGGCAAGAG | TGG |
| 25279043 | 1 | CCAGAAGGAGGGGCAAGAGT | GGG |
| 25279046 | 1 | GAAGGAGGGGCAAGAGTGGG | AGG |
| 25279047 | 1 | AAGGAGGGGCAAGAGTGGGA | GGG |
| 25279048 | 1 | AGGAGGGGCAAGAGTGGGAG | GGG |
| 25279049 | 1 | GGAGGGGCAAGAGTGGGAGG | GGG |
| 25279068 | 1 | GGGGCGCAGATCCAGAATCA | CGG |
| 25279068 | −1 | GTCAGCTGCCTCCGTGATTC | TGG |
| 25279071 | 1 | GCGCAGATCCAGAATCACGG | AGG |
| 25279082 | 1 | GAATCACGGAGGCAGCTGAC | CGG |
| 25279085 | 1 | TCACGGAGGCAGCTGACCGG | AGG |
| 25279088 | 1 | CGGAGGCAGCTGACCGGAGG | AGG |
| 25279090 | −1 | CCTTGGGCAGCTGCCTCCTC | CGG |
| 25279101 | 1 | CCGGAGGAGGCAGCTGCCCA | AGG |
| 25279102 | 1 | CGGAGGAGGCAGCTGCCCAA | GGG |
| 25279103 | 1 | GGAGGAGGCAGCTGCCCAAG | GGG |
| 25279106 | −1 | CCTTCTGAGTCCATCCCCTT | GGG |
| 25279107 | 1 | GAGGCAGCTGCCCAAGGGGA | TGG |
| 25279107 | −1 | GCCTTCTGAGTCCATCCCCT | TGG |
| 25279117 | 1 | CCCAAGGGGATGGACTCAGA | AGG |
| 25279129 | −1 | TCGTTTGGATAACAGCACTT | TGG |
| 25279144 | −1 | CCACTTGCAAAGAGTTCGTT | TGG |
| 25279155 | 1 | CCAAACGAACTCTTTGCAAG | TGG |
| 25279170 | 1 | GCAAGTGGTCTCTTTGCAAC | agg |
| 25279175 | 1 | TGGTCTCTTTGCAACaggcc | tgg |
| 25279176 | 1 | GGTCTCTTTGCAACaggcct | ggg |
| 25279177 | 1 | GTCTCTTTGCAACaggcctg | ggg |
| 25279178 | 1 | TCTCTTTGCAACaggcctgg | ggg |
| 25279182 | −1 | aggcaagactgctctccccc | agg |
| 25279202 | −1 | ctgattagcggtgtgacttt | agg |
| 25279214 | −1 | CCGTGCCGgccgctgattag | cgg |
| 25279216 | 1 | aaagtcacaccgctaatcag | cgg |
| 25279220 | 1 | tcacaccgctaatcagcggc | CGG |
| 25279225 | 1 | ccgctaatcagcggcCGGCA | CGG |
| 25279226 | 1 | cgctaatcagcggcCGGCAC | GGG |
| 25279227 | 1 | gctaatcagcggcCGGCACG | GGG |
| 25279228 | −1 | tagtaactgttACCCCGTGC | CGg |
| 25279264 | 1 | actcactacgtacccaatgc | tgg |
| 25279265 | 1 | ctcactacgtacccaatgct | ggg |
| 25279265 | −1 | aagtcacttcgcccagcatt | ggg |
| 25279266 | −1 | caagtcacttcgcccagcat | tgg |
| 25279295 | −1 | gccatgagcattgagctcgc | tgg |
| 25279305 | 1 | gccagcgagctcaatgctca | tgg |
| 25279321 | −1 | aaacaatgccagctgctcag | agg |
| 25279324 | 1 | atggcaatcctctgagcagc | tgg |
| 25279354 | 1 | tcatctcaattttacagctc | agg |
| 25279361 | 1 | aattttacagctcaggaagc | tgg |
| 25279362 | 1 | attttacagctcaggaagct | ggg |
| 25279371 | 1 | ctcaggaagctgggacacag | agG |
| 25279381 | 1 | tgggacacagagGAAGAGCC | AGG |
| 25279388 | −1 | GGTTGTCAGTGTTCAGAGCC | TGG |
| 25279409 | −1 | ACAGTGTGGGTCTCTCAATC | AGG |
| 25279422 | −1 | GTAACGGTGATGAACAGTGT | GGG |
| 25279423 | −1 | CGTAACGGTGATGAACAGTG | TGG |
| 25279438 | −1 | ATACAGCATATATAGCGTAA | CGG |
| 25279456 | 1 | GCTATATATGCTGTATAGAA | AGG |
| 25279460 | 1 | TATATGCTGTATAGAAAGGc | agg |
| 25279464 | 1 | TGCTGTATAGAAAGGcagga | tgg |
| 25279472 | 1 | AGAAAGGcaggatggcataa | tgg |
| 25279483 | 1 | atggcataatggttaaacct | agg |
| 25279487 | 1 | cataatggttaaacctaggt | agg |
| 25279489 | −1 | gattcaaaccctacctacct | agg |
| 25279491 | 1 | atggttaaacctaggtaggt | agg |
| 25279492 | 1 | tggttaaacctaggtaggta | ggg |
| 25279511 | −1 | agctagtaaatggtagcagg | agg |
| 25279514 | −1 | cagagctagtaaatggtagc | agg |
| 25279521 | −1 | caagtcacagagctagtaaa | tgg |
| 25279533 | 1 | catttactagctctgtgact | tgg |
| 25279558 | −1 | ggggaaagggaggcacagag | agg |
| 25279568 | −1 | ttttagagatggggaaaggg | agg |
| 25279571 | −1 | ccattttagagatggggaaa | ggg |
| 25279572 | −1 | cccattttagagatggggaa | agg |
| 25279577 | −1 | ttatccccattttagagatg | ggg |
| 25279578 | −1 | attatccccattttagagat | ggg |
| 25279579 | −1 | tattatccccattttagaga | tgg |
| 25279582 | 1 | ccctttccccatctctaaaa | tgg |
| 25279583 | 1 | cctttccccatctctaaaat | ggg |
| 25279584 | 1 | ctttccccatctctaaaatg | ggg |
| 25279609 | −1 | ccacaacagcctcaggtagg | agg |
| 25279611 | 1 | taaatcgtacctcctacctg | agg |
| 25279612 | −1 | agcccacaacagcctcaggt | agg |
| 25279616 | −1 | acttagcccacaacagcctc | agg |
| 25279620 | 1 | cctcctacctgaggctgttg | tgg |
| 25279621 | 1 | ctcctacctgaggctgttgt | ggg |
| 25279635 | 1 | tgttgtgggctaagtctgta | agg |
| 25279654 | 1 | aaggcacgtagaacagtgcc | tgg |
| 25279661 | 1 | gtagaacagtgcctggaacg | tgg |
| 25279661 | −1 | TAGACAGTACCccacgttcc | agg |
| 25279662 | 1 | tagaacagtgcctggaacgt | ggG |
| 25279663 | 1 | agaacagtgcctggaacgtg | gGG |
| 25279692 | −1 | CTCACCATTGTTGTAACAGC | AGG |
| 25279699 | 1 | TGTGCCTGCTGTTACAACAA | TGG |
| 25279720 | −1 | TAGTTCAGCAGCGAGAGATA | AGG |
| 25279736 | 1 | TCTCTCGCTGCTGAACTACC | AGG |
| 25279743 | −1 | TTGCAGAAAGAAGTCTAACC | TGG |
| 25279763 | 1 | ACTTCTTTCTGCAAGTCATG | AGG |
| 25279786 | 1 | CTTTCATAAACTTTTCCTGA | AGG |
| 25279790 | −1 | ACATTCTACGGAAAGCCTTC | AGG |
| 25279802 | −1 | GAGGGGAATTGTACATTCTA | CGG |
| 25279816 | 1 | TAGAATGTACAATTCCCCTC | TGG |
| 25279817 | 1 | AGAATGTACAATTCCCCTCT | GGG |
| 25279819 | −1 | GCCCATGCCTGGACCCAGAG | GGG |
| 25279820 | −1 | CGCCCATGCCTGGACCCAGA | GGG |
| 25279821 | −1 | GCGCCCATGCCTGGACCCAG | AGG |
| 25279823 | 1 | TACAATTCCCCTCTGGGTCC | AGG |
| 25279828 | 1 | TTCCCCTCTGGGTCCAGGCA | TGG |
| 25279829 | 1 | TCCCCTCTGGGTCCAGGCAT | GGG |
| 25279830 | −1 | GCTACCCGGGCGCCCATGCC | TGG |
| 25279836 | 1 | TGGGTCCAGGCATGGGCGCC | CGG |
| 25279837 | 1 | GGGTCCAGGCATGGGCGCCC | GGG |
| 25279843 | −1 | AAGAAGTGGATGTGCTACCC | GGG |
| 25279844 | −1 | TAAGAAGTGGATGTGCTACC | CGG |
| 25279857 | −1 | TGTTCAGGGGTGATAAGAAG | TGG |
| 25279870 | −1 | ATGGGCTCTAAGGTGTTCAG | GGG |
| 25279871 | −1 | GATGGGCTCTAAGGTGTTCA | GGG |
| 25279872 | −1 | TGATGGGCTCTAAGGTGTTC | AGG |
| 25279880 | −1 | TGATAAGCTGATGGGCTCTA | AGG |
| 25279888 | −1 | TGCTGGTTTGATAAGCTGAT | GGG |
| 25279889 | −1 | CTGCTGGTTTGATAAGCTGA | TGG |
| 25279905 | −1 | TCTGCACTCACATCAGCTGC | TGG |
| 25279931 | 1 | AGTGCAGAGCAGACTGTGAG | AGG |
| 25279934 | 1 | GCAGAGCAGACTGTGAGAGG | TGG |
| 25279937 | 1 | GAGCAGACTGTGAGAGGTGG | AGG |
| 25279952 | 1 | GGTGGAGGCTGATACCAGTG | AGG |
| 25279955 | −1 | CCAGCTTGGAGCATCCTCAC | TGG |
| 25279966 | 1 | CCAGTGAGGATGCTCCAAGC | TGG |
| 25279967 | 1 | CAGTGAGGATGCTCCAAGCT | GGG |
| 25279969 | −1 | TTCAGGGCTGGGTCCCAGCT | TGG |
| 25279980 | −1 | TGGGCTCCCGCTTCAGGGCT | GGG |
| 25279981 | −1 | CTGGGCTCCCGCTTCAGGGC | TGG |
| 25279984 | 1 | GCTGGGACCCAGCCCTGAAG | CGG |
| 25279985 | 1 | CTGGGACCCAGCCCTGAAGC | GGG |
| 25279985 | −1 | TTATCTGGGCTCCCGCTTCA | GGG |
| 25279986 | −1 | ATTATCTGGGCTCCCGCTTC | AGG |
| 25279999 | 1 | TGAAGCGGGAGCCCAGATAA | TGG |
| 25279999 | −1 | TTTCCACCCATCCATTATCT | GGG |
| 25280000 | −1 | ATTTCCACCCATCCATTATC | TGG |
| 25280003 | 1 | GCGGGAGCCCAGATAATGGA | TGG |
| 25280004 | 1 | CGGGAGCCCAGATAATGGAT | GGG |
| 25280007 | 1 | GAGCCCAGATAATGGATGGG | TGG |
| 25280013 | 1 | AGATAATGGATGGGTGGAAA | TGG |
| 25280014 | 1 | GATAATGGATGGGTGGAAAT | GGG |
| 25280019 | 1 | TGGATGGGTGGAAATGGGCC | TGG |
| 25280026 | −1 | TCCCACTTCTCCTGGGCTCC | AGG |
| 25280027 | 1 | TGGAAATGGGCCTGGAGCCC | AGG |
| 25280033 | −1 | CTCATCCTCCCACTTCTCCT | GGG |
| 25280034 | −1 | CCTCATCCTCCCACTTCTCC | TGG |
| 25280035 | 1 | GGCCTGGAGCCCAGGAGAAG | TGG |
| 25280036 | 1 | GCCTGGAGCCCAGGAGAAGT | GGG |
| 25280039 | 1 | TGGAGCCCAGGAGAAGTGGG | AGG |
| 25280045 | 1 | CCAGGAGAAGTGGGAGGATG | AGG |
| 25280046 | 1 | CAGGAGAAGTGGGAGGATGA | GGG |
| 25280047 | 1 | AGGAGAAGTGGGAGGATGAG | GGG |
| 25280048 | 1 | GGAGAAGTGGGAGGATGAGG | GGG |
| 25280052 | 1 | AAGTGGGAGGATGAGGGGGC | AGG |
| 25280053 | 1 | AGTGGGAGGATGAGGGGGCA | GGG |
| 25280054 | 1 | GTGGGAGGATGAGGGGGCAG | GGG |
| 25280055 | 1 | TGGGAGGATGAGGGGGCAGG | GGG |
| 25280058 | 1 | GAGGATGAGGGGGCAGGGGG | AGG |
| 25280075 | −1 | AGGAAATAACATTTGATTTC | AGG |
| 25280095 | −1 | TCATGCACCCCAAACTGGTC | AGG |
| 25280097 | 1 | ATGTTATTTCCTGACCAGTT | TGG |
| 25280098 | 1 | TGTTATTTCCTGACCAGTTT | GGG |
| 25280099 | 1 | GTTATTTCCTGACCAGTTTG | GGG |
| 25280100 | −1 | AGAGCTCATGCACCCCAAAC | TGG |
| 25280126 | 1 | TGAGCTCTGTCAACAGCTCA | TGG |
| 25280147 | −1 | CAGCCAACAAGATGAAATTA | GGG |
| 25280148 | −1 | TCAGCCAACAAGATGAAATT | AGG |
| 25280155 | 1 | CTGCCCTAATTTCATCTTGT | TGG |
| 25280161 | 1 | TAATTTCATCTTGTTGGCTG | AGG |
| 25280179 | 1 | TGAGGCACAATTCCTCTCTC | AGG |
| 25280180 | 1 | GAGGCACAATTCCTCTCTCA | GGG |
| 25280180 | −1 | CTCTACACTGTCCCTGAGAG | AGG |
| 25280197 | 1 | TCAGGGACAGTGTAGAGCCT | TGG |
| 25280198 | 1 | CAGGGACAGTGTAGAGCCTT | GGG |
| 25280199 | 1 | AGGGACAGTGTAGAGCCTTG | GGG |
| 25280202 | 1 | GACAGTGTAGAGCCTTGGGG | AGG |
| 25280203 | −1 | GCTCAGGGCCTTCCTCCCCA | AGG |
| 25280206 | 1 | GTGTAGAGCCTTGGGGAGGA | AGG |
| 25280218 | −1 | ATTCCAGGTATACGCGCTCA | GGG |
| 25280219 | −1 | GATTCCAGGTATACGCGCTC | AGG |
| 25280226 | 1 | AGGCCCTGAGCGCGTATACC | TGG |
| 25280233 | 1 | GAGCGCGTATACCTGGAATC | AGG |
| 25280233 | −1 | GATCCCGATTCCCTGATTCC | AGG |
| 25280234 | 1 | AGCGCGTATACCTGGAATCA | GGG |
| 25280240 | 1 | TATACCTGGAATCAGGGAAT | CGG |
| 25280241 | 1 | ATACCTGGAATCAGGGAATC | GGG |
| 25280247 | 1 | GGAATCAGGGAATCGGGATC | AGG |
| 25280248 | 1 | GAATCAGGGAATCGGGATCA | GGG |
| 25280249 | 1 | AATCAGGGAATCGGGATCAG | GGG |
| 25280272 | −1 | TCCTGGGTGGGGGCTTTATT | GGG |
| 25280273 | −1 | ATCCTGGGTGGGGGCTTTAT | TGG |
| 25280282 | 1 | GCCCAATAAAGCCCCCACCC | AGG |
| 25280282 | −1 | AGTCAGAGGATCCTGGGTGG | GGG |
| 25280283 | −1 | AAGTCAGAGGATCCTGGGTG | GGG |
| 25280284 | −1 | GAAGTCAGAGGATCCTGGGT | GGG |
| 25280285 | −1 | GGAAGTCAGAGGATCCTGGG | TGG |
| 25280288 | −1 | TGAGGAAGTCAGAGGATCCT | GGG |
| 25280289 | −1 | ATGAGGAAGTCAGAGGATCC | TGG |
| 25280296 | −1 | aaaaGAGATGAGGAAGTCAG | AGG |
| 25280306 | −1 | aaaaaaaaaaaaaaGAGATG | AGG |
| 25280346 | 1 | gcagtctcactctgtcatcc | agg |
| 25280350 | 1 | tctcactctgtcatccaggc | tgg |
| 25280353 | −1 | cgcaccactgtactccagcc | tgg |
| 25280360 | 1 | tcatccaggctggagtacag | tgg |
| 25280371 | 1 | ggagtacagtggtgcgatct | cgg |
| 25280393 | −1 | cgcttgaacccagaaggctg | agg |
| 25280395 | 1 | tcactgcaacctcagccttc | tgg |
| 25280396 | 1 | cactgcaacctcagccttct | ggg |
| 25280399 | −1 | gagaatcgcttgaacccaga | agg |
| 25280421 | −1 | gctactcaggaggctgaggc | agg |
| 25280425 | −1 | cccagctactcaggaggctg | agg |
| 25280431 | −1 | tgtaatcccagctactcagg | agg |
| 25280434 | −1 | gcctgtaatcccagctactc | agg |
| 25280435 | 1 | gcctcagcctcctgagtagc | tgg |
| 25280436 | 1 | cctcagcctcctgagtagct | ggg |
| 25280444 | 1 | tcctgagtagctgggattac | agg |
| 25280462 | −1 | caaaaattagcctggcatgg | tgg |
| 25280463 | 1 | caggcatgcgccaccatgcc | agg |
| 25280465 | −1 | atacaaaaattagcctggca | tgg |
| 25280470 | −1 | taaaaatacaaaaattagcc | tgg |
| 25280491 | 1 | ttttgtatttttagtagaga | cgg |
| 25280492 | 1 | tttgtatttttagtagagac | ggg |
| 25280493 | 1 | ttgtatttttagtagagacg | ggg |
| 25280507 | 1 | gagacggggtttcaccatgt | tgg |
| 25280510 | −1 | tgagaccagcctggccaaca | tgg |
| 25280512 | 1 | ggggtttcaccatgttggcc | agg |
| 25280516 | 1 | tttcaccatgttggccaggc | tgg |
| 25280519 | −1 | tcaggagtttgagaccagcc | tgg |
| 25280537 | −1 | tgggcagatcacttgaagtc | agg |
| 25280556 | −1 | gcactttgggaggctgaggt | ggg |
| 25280557 | −1 | agcactttgggaggctgagg | tgg |
| 25280560 | −1 | cctagcactttgggaggctg | agg |
| 25280566 | −1 | tgtaatcctagcactttggg | agg |
| 25280569 | −1 | gtctgtaatcctagcacttt | ggg |
| 25280570 | −1 | tgtctgtaatcctagcactt | tgg |
| 25280571 | 1 | cctcagcctcccaaagtgct | agg |
| 25280597 | −1 | aaaaaaaaggccaggcacag | tgg |
| 25280598 | 1 | cagacataagccactgtgcc | tgg |
| 25280605 | −1 | aaaaaaaaaaaaaaaaggcc | agg |
| 25280629 | 1 | ttttttttttttttgtaaac | agg |
| 25280630 | 1 | tttttttttttttgtaaaca | ggg |
| 25280645 | −1 | ccagcagcctgggtgacaga | ggg |
| 25280646 | −1 | tccagcagcctgggtgacag | agg |
| 25280649 | 1 | agggtctccctctgtcaccc | agg |
| 25280655 | −1 | ccactacactccagcagcct | ggg |
| 25280656 | 1 | ccctctgtcacccaggctgc | tgg |
| 25280656 | −1 | accactacactccagcagcc | tgg |
| 25280666 | 1 | cccaggctgctggagtgtag | tgg |
| 25280683 | −1 | gttaaggctgcagtgagctg | cgg |
| 25280699 | −1 | ggcttgtgcctagaaggtta | agg |
| 25280702 | 1 | cactgcagccttaaccttct | agg |
| 25280705 | −1 | gaggatggcttgtgcctaga | agg |
| 25280720 | −1 | aggagggtgaggtaggagga | tgg |
| 25280724 | −1 | actcaggagggtgaggtagg | agg |
| 25280727 | −1 | gctactcaggagggtgaggt | agg |
| 25280731 | −1 | cccagctactcaggagggtg | agg |
| 25280736 | −1 | gtagtcccagctactcagga | ggg |
| 25280737 | −1 | tgtagtcccagctactcagg | agg |
| 25280740 | −1 | gcctgtagtcccagctactc | agg |
| 25280741 | 1 | acctcaccctcctgagtagc | tgg |
| 25280742 | 1 | cctcaccctcctgagtagct | ggg |
| 25280750 | 1 | tcctgagtagctgggactac | agg |
| 25280768 | −1 | acaaaattacttgggcgtgg | tgg |
| 25280771 | −1 | aatacaaaattacttgggcg | tgg |
| 25280776 | −1 | caaaaaatacaaaattactt | ggg |
| 25280777 | −1 | acaaaaaatacaaaattact | tgg |
| 25280798 | 1 | ttgtattttttgtagagaca | agg |
| 25280817 | 1 | aaggtcttgctatgttgcct | agg |
| 25280821 | 1 | tcttgctatgttgcctaggc | tgg |
| 25280823 | −1 | gaggagttcaagaccagcct | agg |
| 25280842 | −1 | agggaggattgcttgagctg | agg |
| 25280858 | −1 | ctttgggaggccaaggaggg | agg |
| 25280859 | 1 | ctcaagcaatcctccctcct | tgg |
| 25280861 | −1 | gcactttgggaggccaagga | ggg |
| 25280862 | −1 | agcactttgggaggccaagg | agg |
| 25280865 | −1 | cccagcactttgggaggcca | agg |
| 25280871 | −1 | cacaatcccagcactttggg | agg |
| 25280874 | −1 | cagcacaatcccagcacttt | ggg |
| 25280875 | 1 | tccttggcctcccaaagtgc | tgg |
| 25280875 | −1 | ccagcacaatcccagcactt | tgg |
| 25280876 | 1 | ccttggcctcccaaagtgct | ggg |
| 25280886 | 1 | ccaaagtgctgggattgtgc | tgg |
| 25280887 | 1 | caaagtgctgggattgtgct | ggg |
| 25280895 | 1 | tgggattgtgctgggattac | agg |
| 25280913 | −1 | GGAAGTCAgaccaggtatgg | tgg |
| 25280914 | 1 | caggtgtgagccaccatacc | tgg |
| 25280916 | −1 | TTAGGAAGTCAgaccaggta | tgg |
| 25280921 | −1 | AAAGATTAGGAAGTCAgacc | agg |
| 25280934 | −1 | GAGTTGGGGCCCTAAAGATT | AGG |
| 25280935 | 1 | ggtcTGACTTCCTAATCTTT | AGG |
| 25280936 | 1 | gtcTGACTTCCTAATCTTTA | GGG |
| 25280948 | −1 | CCTGGATAAGGGCAGAGTTG | GGG |
| 25280949 | −1 | GCCTGGATAAGGGCAGAGTT | GGG |
| 25280950 | −1 | TGCCTGGATAAGGGCAGAGT | TGG |
| 25280959 | 1 | CCCCAACTCTGCCCTTATCC | AGG |
| 25280959 | −1 | GAGGAGAGTTGCCTGGATAA | GGG |
| 25280960 | −1 | AGAGGAGAGTTGCCTGGATA | AGG |
| 25280966 | −1 | ATGGGGAGAGGAGAGTTGCC | TGG |
| 25280978 | −1 | AGTTAGTGGAAGATGGGGAG | AGG |
| 25280983 | −1 | aAAGAAGTTAGTGGAAGATG | GGG |
| 25280984 | −1 | caAAGAAGTTAGTGGAAGAT | GGG |
| 25280985 | −1 | ccaAAGAAGTTAGTGGAAGA | TGG |
| 25280992 | −1 | gaatattccaAAGAAGTTAG | TGG |
| 25280996 | 1 | CCATCTTCCACTAACTTCTT | tgg |
| 25281014 | −1 | ctctaaggcttttacagctc | tgg |
| 25281029 | −1 | gttggacttgatactctcta | agg |
| 25281047 | −1 | tgtctgtaacacataggagt | tgg |
| 25281053 | −1 | tttccctgtctgtaacacat | agg |
| 25281060 | 1 | aactcctatgtgttacagac | agg |
| 25281061 | 1 | actcctatgtgttacagaca | ggg |
| 25281070 | 1 | tgttacagacagggaaactg | agg |
| 25281080 | 1 | agggaaactgaggcctaaag | agg |
| 25281081 | 1 | gggaaactgaggcctaaaga | ggg |
| 25281082 | −1 | gcaagtccattaccctcttt | agg |
| 25281087 | 1 | ctgaggcctaaagagggtaa | tgg |
| 25281104 | −1 | tcacctcactaagtgatctt | agg |
| 25281112 | 1 | ttgcctaagatcacttagtg | agg |
| 25281149 | −1 | ACTATGTCCTTGCACAGGCT | AGG |
| 25281153 | 1 | gaGACAGCCTAGCCTGTGCA | AGG |
| 25281154 | −1 | CTGGAACTATGTCCTTGCAC | AGG |
| 25281166 | 1 | CTGTGCAAGGACATAGTTCC | AGG |
| 25281173 | −1 | AGAGCCCAGCTCTGAATGCC | TGG |
| 25281179 | 1 | TAGTTCCAGGCATTCAGAGC | TGG |
| 25281180 | 1 | AGTTCCAGGCATTCAGAGCT | GGG |
| 25281192 | 1 | TCAGAGCTGGGCTCTGCTGC | CGG |
| 25281200 | −1 | CTACCAGGCCCCAAACATGC | CGG |
| 25281201 | 1 | GGCTCTGCTGCCGGCATGTT | TGG |
| 25281202 | 1 | GCTCTGCTGCCGGCATGTTT | GGG |
| 25281203 | 1 | CTCTGCTGCCGGCATGTTTG | GGG |
| 25281208 | 1 | CTGCCGGCATGTTTGGGGCC | TGG |
| 25281215 | −1 | TCAGCAGTGAACTAACTACC | AGG |
| 25281235 | 1 | TAGTTCACTGCTGAACTACC | AGG |
| 25281242 | −1 | TGGAGAAAGAAAATCTAACC | TGG |
| 25281261 | 1 | GATTTTCTTTCTCCAAGTTG | TGG |
| 25281262 | −1 | TTTATGAAAGCTCCACAACT | TGG |
| 25281286 | 1 | CTTTCATAAACTTTTCCTGA | AGG |
| 25281290 | −1 | ACATTGTAAGGAAGACCTTC | AGG |
| 25281302 | −1 | GAGGAGAATTGTACATTGTA | AGG |
| 25281316 | 1 | TACAATGTACAATTCTCCTC | TGG |
| 25281317 | 1 | ACAATGTACAATTCTCCTCT | GGG |
| 25281321 | −1 | GCGCTCATGACCGGGCCCAG | AGG |
| 25281322 | 1 | GTACAATTCTCCTCTGGGCC | CGG |
| 25281329 | −1 | TGTGAGGGGCGCTCATGACC | GGG |
| 25281330 | −1 | CTGTGAGGGGCGCTCATGAC | CGG |
| 25281342 | 1 | CGGTCATGAGCGCCCCTCAC | AGG |
| 25281343 | −1 | GACCAGAGAGAGCCTGTGAG | GGG |
| 25281344 | −1 | GGACCAGAGAGAGCCTGTGA | GGG |
| 25281345 | −1 | GGGACCAGAGAGAGCCTGTG | AGG |
| 25281352 | 1 | CGCCCCTCACAGGCTCTCTC | TGG |
| 25281365 | −1 | TTCCTCTCATTTTACAGAAG | GGG |
| 25281366 | −1 | TTTCCTCTCATTTTACAGAA | GGG |
| 25281367 | −1 | TTTTCCTCTCATTTTACAGA | AGG |
| 25281374 | 1 | GTCCCCTTCTGTAAAATGAG | AGG |
| 25281381 | 1 | TCTGTAAAATGAGAGGAAAA | TGG |
| 25281401 | 1 | TGGAAGAATTGCTCTACTCA | TGG |
| 25281419 | 1 | CATGGAATCTTCAATAAGTC | TGG |
| 25281420 | 1 | ATGGAATCTTCAATAAGTCT | GGG |
| 25281432 | 1 | GTAGCAATGCTATATGCATA | GGG |
| 25281433 | −1 | TGTAGCAATGCTATATGCAT | AGG |
| 25281450 | 1 | CATATAGCATTGCTACAAAA | TGG |
| 25281484 | 1 | TAACAATCGTGTTTAATAAA | AGG |
| 25281488 | 1 | AATCGTGTTTAATAAAAGGT | TGG |
| 25281507 | 1 | TTGGATTTGCATATCTGAAG | Tgg |
| 25281508 | 1 | TGGATTTGCATATCTGAAGT | ggg |
| 25281509 | 1 | GGATTTGCATATCTGAAGTg | ggg |
| 25281531 | −1 | cagtgaggcttgtgttcagt | tgg |
| 25281546 | −1 | gtgcacatgcgggagcagtg | agg |
| 25281556 | −1 | tgaaggtgcagtgcacatgc | ggg |
| 25281557 | −1 | atgaaggtgcagtgcacatg | cgg |
| 25281573 | −1 | agcaggaaatatgtatatga | agg |
| 25281587 | 1 | tcatatacatatttcctgct | tgg |
| 25281590 | −1 | aattccctcaggagccaagc | agg |
| 25281596 | 1 | tatttcctgcttggctcctg | agg |
| 25281597 | 1 | atttcctgcttggctcctga | ggg |
| 25281601 | −1 | GGGATTactcaaattccctc | agg |
| 25281618 | 1 | ggaatttgagtAATCCCAAG | AGG |
| 25281621 | −1 | TTTCTACAGGGGTTCCTCTT | GGG |
| 25281622 | −1 | TTTTCTACAGGGGTTCCTCT | TGG |
| 25281632 | −1 | CCAGGGGACATTTTCTACAG | GGG |
| 25281633 | −1 | GCCAGGGGACATTTTCTACA | GGG |
| 25281634 | −1 | GGCCAGGGGACATTTTCTAC | AGG |
| 25281643 | 1 | CCCCTGTAGAAAATGTCCCC | TGG |
| 25281648 | −1 | GAATGGGGGTGTGTGGCCAG | GGG |
| 25281649 | −1 | GGAATGGGGGTGTGTGGCCA | GGG |
| 25281650 | −1 | AGGAATGGGGGTGTGTGGCC | AGG |
| 25281655 | −1 | TCCTTAGGAATGGGGGTGTG | TGG |
| 25281662 | −1 | GCTTGCATCCTTAGGAATGG | GGG |
| 25281663 | −1 | TGCTTGCATCCTTAGGAATG | GGG |
| 25281664 | −1 | CTGCTTGCATCCTTAGGAAT | GGG |
| 25281665 | 1 | GCCACACACCCCCATTCCTA | AGG |
| 25281665 | −1 | CCTGCTTGCATCCTTAGGAA | TGG |
| 25281670 | −1 | TATCTCCTGCTTGCATCCTT | AGG |
| 25281676 | 1 | CCATTCCTAAGGATGCAAGC | AGG |
| 25281701 | −1 | ACAACAAGGAGGGAGGTGCA | GGG |
| 25281702 | −1 | GACAACAAGGAGGGAGGTGC | AGG |
| 25281708 | −1 | TCTTCTGACAACAAGGAGGG | AGG |
| 25281711 | −1 | ACTTCTTCTGACAACAAGGA | GGG |
| 25281712 | −1 | CACTTCTTCTGACAACAAGG | AGG |
| 25281715 | −1 | TTGCACTTCTTCTGACAACA | AGG |
| 25281748 | −1 | GTGAGAAGTGGGCATTAGGA | AGG |
| 25281752 | −1 | GTGGGTGAGAAGTGGGCATT | AGG |
| 25281759 | −1 | TTGGGGCGTGGGTGAGAAGT | GGG |
| 25281760 | −1 | TTTGGGGCGTGGGTGAGAAG | TGG |
| 25281770 | −1 | GACCTGGGGATTTGGGGCGT | GGG |
| 25281771 | −1 | GGACCTGGGGATTTGGGGCG | TGG |
| 25281776 | −1 | CCATGGGACCTGGGGATTTG | GGG |
| 25281777 | −1 | TCCATGGGACCTGGGGATTT | GGG |
| 25281778 | −1 | CTCCATGGGACCTGGGGATT | TGG |
| 25281779 | 1 | CACCCACGCCCCAAATCCCC | AGG |
| 25281784 | −1 | AAGGACCTCCATGGGACCTG | GGG |
| 25281785 | −1 | CAAGGACCTCCATGGGACCT | GGG |
| 25281786 | −1 | CCAAGGACCTCCATGGGACC | TGG |
| 25281787 | 1 | CCCCAAATCCCCAGGTCCCA | TGG |
| 25281790 | 1 | CAAATCCCCAGGTCCCATGG | AGG |
| 25281792 | −1 | AGGCCCCCAAGGACCTCCAT | GGG |
| 25281793 | −1 | GAGGCCCCCAAGGACCTCCA | TGG |
| 25281797 | 1 | CCAGGTCCCATGGAGGTCCT | TGG |
| 25281798 | 1 | CAGGTCCCATGGAGGTCCTT | GGG |
| 25281799 | 1 | AGGTCCCATGGAGGTCCTTG | GGG |
| 25281800 | 1 | GGTCCCATGGAGGTCCTTGG | GGG |
| 25281803 | −1 | CAGGATATAGGAGGCCCCCA | AGG |
| 25281812 | −1 | TGACACCACCAGGATATAGG | AGG |
| 25281815 | 1 | CTTGGGGGCCTCCTATATCC | TGG |
| 25281815 | −1 | ACCTGACACCACCAGGATAT | AGG |
| 25281818 | 1 | GGGGGCCTCCTATATCCTGG | TGG |
| 25281822 | −1 | CAAATCAACCTGACACCACC | AGG |
| 25281825 | 1 | TCCTATATCCTGGTGGTGTC | AGG |
| 25281834 | 1 | CTGGTGGTGTCAGGTTGATT | TGG |
| 25281858 | −1 | TCTGCCAGAGAGGACAAGGG | AGG |
| 25281861 | −1 | GGGTCTGCCAGAGAGGACAA | GGG |
| 25281862 | −1 | AGGGTCTGCCAGAGAGGACA | AGG |
| 25281865 | 1 | GTGTCCTCCCTTGTCCTCTC | TGG |
| 25281868 | −1 | ATACCCAGGGTCTGCCAGAG | AGG |
| 25281875 | 1 | TTGTCCTCTCTGGCAGACCC | TGG |
| 25281876 | 1 | TGTCCTCTCTGGCAGACCCT | GGG |
| 25281881 | −1 | TTGAAACATACACATACCCA | GGG |
| 25281882 | −1 | ATTGAAACATACACATACCC | AGG |
| 25281895 | 1 | TGGGTATGTGTATGTTTCAA | TGG |
| 25281946 | 1 | AAAGACTTTTTCTGAGACTT | TGG |
| 25281964 | −1 | CAATGAGAAGCTCTCATTAC | TGG |
| 25281984 | 1 | AGAGCTTCTCATTGTTATCA | AGG |
| 25281989 | 1 | TTCTCATTGTTATCAAGGCC | AGG |
| 25281990 | 1 | TCTCATTGTTATCAAGGCCA | GGG |
| 25281994 | 1 | ATTGTTATCAAGGCCAGGGC | TGG |
| 25281996 | −1 | CTGCCACTGGTCTCCAGCCC | TGG |
| 25282004 | 1 | AGGCCAGGGCTGGAGACCAG | TGG |
| 25282008 | 1 | CAGGGCTGGAGACCAGTGGC | AGG |
| 25282009 | −1 | AATAGGAACTCACCTGCCAC | TGG |
| 25282026 | −1 | ATCATGACAATCACAGCAAT | AGG |
| 25282085 | −1 | ttagtacagtgactggcaca | tgg |
| 25282092 | −1 | ataatgtttagtacagtgac | tgg |
| 25282112 | 1 | gtactaaacattatttcctt | tgg |
| 25282117 | −1 | gaggtttctgggaaatccaa | agg |
| 25282128 | −1 | gacccacctgagaggtttct | ggg |
| 25282129 | −1 | agacccacctgagaggtttc | tgg |
| 25282133 | 1 | ggatttcccagaaacctctc | agg |
| 25282136 | 1 | tttcccagaaacctctcagg | tgg |
| 25282136 | −1 | ggtaattagacccacctgag | agg |
| 25282137 | 1 | ttcccagaaacctctcaggt | ggg |
| 25282157 | −1 | tttccttatcagctgaataa | ggg |
| 25282158 | −1 | ctttccttatcagctgaata | agg |
| 25282165 | 1 | ttacccttattcagctgata | agg |
| 25282192 | 1 | taagcaacttacaagaccac | agg |
| 25282193 | 1 | aagcaacttacaagaccaca | ggg |
| 25282197 | −1 | GTTTccacttcatagccctg | tgg |
| 25282204 | 1 | aagaccacagggctatgaag | tgg |
| 25282275 | 1 | agagtctcactgtgtcgccc | agg |
| 25282279 | 1 | tctcactgtgtcgcccaggc | tgg |
| 25282281 | −1 | gcaccactgcactccagcct | ggg |
| 25282282 | −1 | cgcaccactgcactccagcc | tgg |
| 25282289 | 1 | tcgcccaggctggagtgcag | tgg |
| 25282294 | 1 | caggctggagtgcagtggtg | cgg |
| 25282322 | −1 | cgcttgaacccgggaggcag | agg |
| 25282324 | 1 | tcactgcaacctctgcctcc | cgg |
| 25282325 | 1 | cactgcaacctctgcctccc | ggg |
| 25282328 | −1 | gagaatcgcttgaacccggg | agg |
| 25282331 | −1 | caggagaatcgcttgaaccc | ggg |
| 25282332 | −1 | gcaggagaatcgcttgaacc | cgg |
| 25282350 | −1 | cagctactcgggaggcaggc | agg |
| 25282354 | −1 | atcccagctactcgggaggc | agg |
| 25282358 | −1 | tgtaatcccagctactcggg | agg |
| 25282361 | −1 | acctgtaatcccagctactc | ggg |
| 25282362 | 1 | ctgcctgcctcccgagtagc | tgg |
| 25282362 | −1 | cacctgtaatcccagctact | cgg |
| 25282363 | 1 | tgcctgcctcccgagtagct | ggg |
| 25282371 | 1 | tcccgagtagctgggattac | agg |
| 25282420 | 1 | ttttgtaattttagtagaga | cgg |
| 25282421 | 1 | tttgtaattttagtagagac | ggg |
| 25282422 | 1 | ttgtaattttagtagagacg | ggg |
| 25282436 | 1 | gagacggggtttcaccatgt | tgg |
| 25282439 | −1 | cgagactagcctggccaaca | tgg |
| 25282441 | 1 | ggggtttcaccatgttggcc | agg |
| 25282448 | −1 | tcagcagttcgagactagcc | tgg |
| 25282483 | −1 | Ccaatttaggaggatgaggt | ggg |
| 25282484 | −1 | ACcaatttaggaggatgagg | tgg |
| 25282487 | −1 | GATACcaatttaggaggatg | agg |
| 25282493 | −1 | TATAAAGATACcaatttagg | agg |
| 25282494 | 1 | cccacctcatcctcctaaat | tgG |
| 25282496 | −1 | ACATATAAAGATACcaattt | agg |
| 25282519 | −1 | TTGCCACCAGTTGACTCTTT | TGG |
| 25282524 | 1 | ATATGTCCAAAAGAGTCAAC | TGG |
| 25282527 | 1 | TGTCCAAAAGAGTCAACTGG | TGG |
| 25282540 | 1 | CAACTGGTGGCAATTTAGTG | AGG |
| 25282554 | 1 | TTAGTGAGGTTTAATCTAAt | agg |
| 25282569 | 1 | CTAAtaggaaatgatagagc | tgg |
| 25282570 | 1 | TAAtaggaaatgatagagct | ggg |
| 25282593 | −1 | gcataggttttgagttcaca | tgg |
| 25282609 | −1 | AAAGgtggaaggggaagcat | agg |
| 25282618 | −1 | GTTTTTCAAAAAGgtggaag | ggg |
| 25282619 | −1 | TGTTTTTCAAAAAGgtggaa | ggg |
| 25282620 | −1 | ATGTTTTTCAAAAAGgtgga | agg |
| 25282624 | −1 | GACAATGTTTTTCAAAAAGg | tgg |
| 25282627 | −1 | cTAGACAATGTTTTTCAAAA | AGg |
| 25282639 | 1 | CTTTTTGAAAAACATTGTCT | Agg |
| 25282643 | 1 | TTGAAAAACATTGTCTAggc | tgg |
| 25282644 | 1 | TGAAAAACATTGTCTAggct | ggg |
| 25282652 | 1 | ATTGTCTAggctgggcacga | tgg |
| 25282670 | −1 | tcccaaagtgctgggattac | agg |
| 25282678 | −1 | cctccgtctcccaaagtgct | ggg |
| 25282679 | 1 | tgcctgtaatcccagcactt | tgg |
| 25282679 | −1 | acctccgtctcccaaagtgc | tgg |
| 25282680 | 1 | gcctgtaatcccagcacttt | ggg |
| 25282686 | 1 | aatcccagcactttgggaga | cgg |
| 25282689 | 1 | cccagcactttgggagacgg | agg |
| 25282692 | 1 | agcactttgggagacggagg | tgg |
| 25282693 | 1 | gcactttgggagacggaggt | ggg |
| 25282696 | 1 | ctttgggagacggaggtggg | tgg |
| 25282707 | 1 | ggaggtgggtggattacatg | agg |
| 25282712 | 1 | tgggtggattacatgaggtc | agg |
| 25282730 | 1 | tcaggagttcgagaccagct | tgg |
| 25282733 | −1 | tggctaatttttggccaagc | tgg |
| 25282742 | −1 | caccacgcctggctaatttt | tgg |
| 25282746 | 1 | agcttggccaaaaattagcc | agg |
| 25282751 | 1 | ggccaaaaattagccaggcg | tgg |
| 25282753 | −1 | caggcgcgcgccaccacgcc | tgg |
| 25282754 | 1 | caaaaattagccaggcgtgg | tgg |
| 25282767 | 1 | ggcgtggtggcgcgcgcctg | tgg |
| 25282772 | −1 | tgtgcttcagtgggaaccac | agg |
| 25282781 | −1 | tcagcctcctgtgcttcagt | ggg |
| 25282782 | −1 | ttcagcctcctgtgcttcag | tgg |
| 25282785 | 1 | tgtggttcccactgaagcac | agg |
| 25282788 | 1 | ggttcccactgaagcacagg | agg |
| 25282816 | 1 | gcacaagaatcacttgaacc | cgg |
| 25282817 | 1 | cacaagaatcacttgaaccc | ggg |
| 25282820 | 1 | aagaatcacttgaacccggg | agg |
| 25282823 | 1 | aatcacttgaacccgggagg | tgg |
| 25282823 | −1 | cgctgcaacctccacctccc | ggg |
| 25282824 | −1 | tcgctgcaacctccacctcc | cgg |
| 25282826 | 1 | cacttgaacccgggaggtgg | agg |
| 25282848 | −1 | ggagtgcagtggtgcgatct | cgg |
| 25282859 | −1 | ttgcccaggttggagtgcag | tgg |
| 25282866 | 1 | cgcaccactgcactccaacc | tgg |
| 25282867 | 1 | gcaccactgcactccaacct | ggg |
| 25282869 | −1 | agtctctctgttgcccaggt | tgg |
| 25282873 | −1 | acagagtctctctgttgccc | agg |
| 25282914 | 1 | aaaaaaaattgtctacatgc | tgg |
| 25282966 | −1 | ATATTGTCTCTAAGTTTGGG | AGG |
| 25282969 | −1 | TTAATATTGTCTCTAAGTTT | GGG |
| 25282970 | −1 | ATTAATATTGTCTCTAAGTT | TGG |
| 25282986 | 1 | CTTAGAGACAATATTAATGA | CGG |
| 25283027 | −1 | TTCGCACATGAATAAATGAC | TGG |
| 25283047 | 1 | TATTCATGTGCGAAAACAGT | TGG |
| 25283079 | 1 | ATAAAATAGCTTTTAGAGTT | TGG |
| 25283114 | −1 | attaggttgccagaatcaaa | tgg |
| 25283116 | 1 | ttacatataccatttgattc | tgg |
| 25283130 | 1 | tgattctggcaacctaatga | agg |
| 25283131 | −1 | aatgatcatactccttcatt | agg |
| 25283155 | −1 | tgttcttgtctgttaaatag | ggg |
| 25283156 | −1 | ttgttcttgtctgttaaata | ggg |
| 25283157 | −1 | cttgttcttgtctgttaaat | agg |
| 25283174 | 1 | taacagacaagaacaagaag | agg |
| 25283175 | 1 | aacagacaagaacaagaaga | ggg |
| 25283178 | 1 | agacaagaacaagaagaggg | agg |
| 25283179 | 1 | gacaagaacaagaagaggga | ggG |
| 25283186 | 1 | acaagaagagggaggGCAGa | tgg |
| 25283191 | 1 | aagagggaggGCAGatggtg | tgg |
| 25283201 | 1 | GCAGatggtgtggtagtcta | agg |
| 25283207 | 1 | ggtgtggtagtctaaggcac | agg |
| 25283221 | −1 | tttacacctagataatctgc | tgg |
| 25283226 | 1 | caggctccagcagattatct | agg |
| 25283238 | 1 | gattatctaggtgtaaatct | tgg |
| 25283245 | 1 | taggtgtaaatcttggctgt | agg |
| 25283250 | 1 | gtaaatcttggctgtaggcc | agg |
| 25283257 | −1 | cagacatgagccacagggcc | tgg |
| 25283258 | 1 | tggctgtaggccaggccctg | tgg |
| 25283262 | −1 | gattacagacatgagccaca | ggg |
| 25283263 | −1 | ggattacagacatgagccac | agg |
| 25283284 | −1 | cctcggtttcccaaagtgat | ggg |
| 25283285 | 1 | tgtctgtaatcccatcactt | tgg |
| 25283285 | −1 | acctcggtttcccaaagtga | tgg |
| 25283286 | 1 | gtctgtaatcccatcacttt | ggg |
| 25283295 | 1 | cccatcactttgggaaaccg | agg |
| 25283298 | 1 | atcactttgggaaaccgagg | tgg |
| 25283299 | 1 | tcactttgggaaaccgaggt | ggg |
| 25283301 | −1 | ctcaagtgatctgcccacct | cgg |
| 25283313 | 1 | cgaggtgggcagatcacttg | agg |
| 25283318 | 1 | tgggcagatcacttgaggtc | agg |
| 25283336 | 1 | tcaggagttcgagaccagct | tgg |
| 25283339 | −1 | tttcgctatgttggccaagc | tgg |
| 25283348 | −1 | gagaaggggtttcgctatgt | tgg |
| 25283362 | −1 | ttgtatttttaatagagaag | ggg |
| 25283363 | −1 | tttgtatttttaatagagaa | ggg |
| 25283364 | −1 | ttttgtatttttaatagaga | agg |
| 25283384 | 1 | ttaaaaatacaaaaattagc | cgg |
| 25283385 | 1 | taaaaatacaaaaattagcc | ggg |
| 25283390 | 1 | atacaaaaattagccgggca | cgg |
| 25283392 | −1 | caggtgcctgccaccgtgcc | cgg |
| 25283393 | 1 | caaaaattagccgggcacgg | tgg |
| 25283397 | 1 | aattagccgggcacggtggc | agg |
| 25283411 | −1 | tcccaagtagctgggattac | agg |
| 25283419 | −1 | cctcagcctcccaagtagct | ggg |
| 25283420 | 1 | cacctgtaatcccagctact | tgg |
| 25283420 | −1 | gcctcagcctcccaagtagc | tgg |
| 25283421 | 1 | acctgtaatcccagctactt | ggg |
| 25283424 | 1 | tgtaatcccagctacttggg | agg |
| 25283430 | 1 | cccagctacttgggaggctg | agg |
| 25283434 | 1 | gctacttgggaggctgaggc | agg |
| 25283453 | 1 | caggagaatcacttgaaccc | agg |
| 25283456 | 1 | gagaatcacttgaacccagg | agg |
| 25283459 | −1 | cactgcaacctctgcctcct | ggg |
| 25283460 | −1 | tcactgcaacctctgcctcc | tgg |
| 25283462 | 1 | cacttgaacccaggaggcag | agg |
| 25283484 | −1 | ggagtacagtggcaagatct | tgg |
| 25283495 | −1 | tcacccaggctggagtacag | tgg |
| 25283502 | 1 | cttgccactgtactccagcc | tgg |
| 25283503 | 1 | ttgccactgtactccagcct | ggg |
| 25283505 | −1 | gtttcactcgtcacccaggc | tgg |
| 25283509 | −1 | tagagtttcactcgtcaccc | agg |
| 25283560 | 1 | aaaatcttagctctacccac | cgg |
| 25283561 | 1 | aaatcttagctctacccacc | ggg |
| 25283562 | 1 | aatcttagctctacccaccg | ggg |
| 25283564 | −1 | gttacgtaacttgccccggt | ggg |
| 25283565 | −1 | cgttacgtaacttgccccgg | tgg |
| 25283568 | −1 | aggcgttacgtaacttgccc | cgg |
| 25283588 | −1 | atatgaaaaccaaggcacag | agg |
| 25283590 | 1 | tacgtaacgcctctgtgcct | tgg |
| 25283596 | −1 | ttttacagatatgaaaacca | agg |
| 25283610 | 1 | tggttttcatatctgtaaaa | tgg |
| 25283636 | −1 | tcacaaccacactttgacgt | ggg |
| 25283637 | −1 | ctcacaaccacactttgacg | tgg |
| 25283641 | 1 | acagcacccacgtcaaagtg | tgg |
| 25283692 | 1 | taaagtgattaaaacagcgt | agg |
| 25283699 | 1 | attaaaacagcgtaggcaca | tgg |
| 25283711 | 1 | taggcacatggtaaacgctt | agg |
| 25283720 | 1 | ggtaaacgcttaggaaatgt | agg |
| 25283775 | 1 | gatcaagatcacacagttag | agg |
| 25283776 | 1 | atcaagatcacacagttaga | ggg |
| 25283790 | −1 | ttgggttcaaatcaggactc | tgg |
| 25283797 | −1 | gacaaacttgggttcaaatc | agg |
| 25283808 | −1 | ctccagaacgagacaaactt | ggg |
| 25283809 | −1 | gctccagaacgagacaaact | tgg |
| 25283817 | 1 | aacccaagtttgtctcgttc | tgg |
| 25283844 | −1 | TTAATTCcagttttgaaaaa | ggg |
| 25283845 | −1 | TTTAATTCcagttttgaaaa | agg |
| 25283849 | 1 | tgctaaccctttttcaaaac | tgG |
| 25283868 | −1 | AAAGCGGAGGGTGAGCACTT | TGG |
| 25283880 | −1 | GAGGGGCCCAGCAAAGCGGA | GGG |
| 25283881 | −1 | GGAGGGGCCCAGCAAAGCGG | AGG |
| 25283884 | 1 | GTGCTCACCCTCCGCTTTGC | TGG |
| 25283884 | −1 | CAGGGAGGGGCCCAGCAAAG | CGG |
| 25283885 | 1 | TGCTCACCCTCCGCTTTGCT | GGG |
| 25283897 | −1 | ACGCACCTGAGGGCAGGGAG | GGG |
| 25283898 | −1 | GACGCACCTGAGGGCAGGGA | GGG |
| 25283899 | −1 | AGACGCACCTGAGGGCAGGG | AGG |
| 25283902 | −1 | AAGAGACGCACCTGAGGGCA | GGG |
| 25283903 | 1 | CTGGGCCCCTCCCTGCCCTC | AGG |
| 25283903 | −1 | GAAGAGACGCACCTGAGGGC | AGG |
| 25283907 | −1 | AGTGGAAGAGACGCACCTGA | GGG |
| 25283908 | −1 | GAGTGGAAGAGACGCACCTG | AGG |
| 25283925 | −1 | AGGCTGCTGTGGCAGGTGAG | TGG |
| 25283932 | −1 | TGAGCAGAGGCTGCTGTGGC | AGG |
| 25283936 | −1 | ACCCTGAGCAGAGGCTGCTG | TGG |
| 25283945 | 1 | TGCCACAGCAGCCTCTGCTC | AGG |
| 25283945 | −1 | CGGTCTCAGACCCTGAGCAG | AGG |
| 25283946 | 1 | GCCACAGCAGCCTCTGCTCA | GGG |
| 25283957 | 1 | CTCTGCTCAGGGTCTGAGAC | CGG |
| 25283958 | 1 | TCTGCTCAGGGTCTGAGACC | GGG |
| 25283963 | 1 | TCAGGGTCTGAGACCGGGAA | AGG |
| 25283965 | −1 | TGGGTAGCCCTCACCTTTCC | CGG |
| 25283968 | 1 | GTCTGAGACCGGGAAAGGTG | AGG |
| 25283969 | 1 | TCTGAGACCGGGAAAGGTGA | GGG |
| 25283978 | 1 | GGGAAAGGTGAGGGCTACCC | AGG |
| 25283981 | 1 | AAAGGTGAGGGCTACCCAGG | TGG |
| 25283984 | −1 | AGAAAACATCAGGGCCACCT | GGG |
| 25283985 | −1 | CAGAAAACATCAGGGCCACC | TGG |
| 25283993 | −1 | CTGGCTGGCAGAAAACATCA | GGG |
| 25283994 | −1 | GCTGGCTGGCAGAAAACATC | AGG |
| 25284008 | −1 | GAGGGACCTGGTGAGCTGGC | TGG |
| 25284012 | −1 | CTGCGAGGGACCTGGTGAGC | TGG |
| 25284013 | 1 | TTTCTGCCAGCCAGCTCACC | AGG |
| 25284020 | −1 | GCCGCCTGCTGCGAGGGACC | TGG |
| 25284026 | −1 | CCCTTTGCCGCCTGCTGCGA | GGG |
| 25284027 | 1 | CTCACCAGGTCCCTCGCAGC | AGG |
| 25284027 | −1 | TCCCTTTGCCGCCTGCTGCG | AGG |
| 25284030 | 1 | ACCAGGTCCCTCGCAGCAGG | CGG |
| 25284036 | 1 | TCCCTCGCAGCAGGCGGCAA | AGG |
| 25284037 | 1 | CCCTCGCAGCAGGCGGCAAA | GGG |
| 25284040 | 1 | TCGCAGCAGGCGGCAAAGGG | AGG |
| 25284041 | 1 | CGCAGCAGGCGGCAAAGGGA | GGG |
| 25284044 | 1 | AGCAGGCGGCAAAGGGAGGG | AGG |
| 25284065 | 1 | GGTTTGCTGTGAAGATTATG | TGG |
| 25284079 | −1 | ggcccagCGCTCTTGTTGTT | GGG |
| 25284080 | −1 | aggcccagCGCTCTTGTTGT | TGG |
| 25284087 | 1 | GTTCCCAACAACAAGAGCGc | tgg |
| 25284088 | 1 | TTCCCAACAACAAGAGCGct | ggg |
| 25284100 | −1 | agaaaagagagggcagagat | agg |
| 25284110 | −1 | caggacacacagaaaagaga | ggg |
| 25284111 | −1 | ccaggacacacagaaaagag | agg |
| 25284122 | 1 | cctctcttttctgtgtgtcc | tgg |
| 25284123 | 1 | ctctcttttctgtgtgtcct | ggg |
| 25284129 | −1 | gaagccaagtgacttgtccc | agg |
| 25284136 | 1 | gtgtcctgggacaagtcact | tgg |
| 25284145 | 1 | gacaagtcacttggcttctg | tgg |
| 25284172 | 1 | attttctcatgtgcccagcc | agg |
| 25284173 | 1 | ttttctcatgtgcccagcca | ggg |
| 25284174 | 1 | tttctcatgtgcccagccag | ggg |
| 25284174 | −1 | TGAGGGccaaccccctggct | ggg |
| 25284175 | 1 | ttctcatgtgcccagccagg | ggg |
| 25284175 | −1 | ATGAGGGccaaccccctggc | tgg |
| 25284179 | 1 | catgtgcccagccagggggt | tgg |
| 25284179 | −1 | GCATATGAGGGccaaccccc | tgg |
| 25284191 | −1 | GCTGCTGTTATTGCATATGA | GGG |
| 25284192 | −1 | TGCTGCTGTTATTGCATATG | AGG |
| 25284219 | −1 | CGCACATGGACACTCAGTAA | AGG |
| 25284233 | −1 | GCACACGTGCTTGACGCACA | TGG |
| 25284268 | 1 | TTACACTTGTTCTTATTATT | AGG |
| 25284299 | −1 | taatgagtgctcagtaaatg | tgg |
| 25284313 | 1 | catttactgagcactcatta | tgg |
| 25284314 | 1 | atttactgagcactcattat | ggg |
| 25284319 | 1 | ctgagcactcattatgggcc | agg |
| 25284326 | −1 | taagcacttagggcagggcc | tgg |
| 25284331 | −1 | ctaattaagcacttagggca | ggg |
| 25284332 | −1 | gctaattaagcacttagggc | agg |
| 25284336 | −1 | taaagctaattaagcactta | ggg |
| 25284337 | −1 | ctaaagctaattaagcactt | agg |
| 25284362 | −1 | ggggataagataaggattag | agg |
| 25284370 | −1 | tgccgtgtggggataagata | agg |
| 25284379 | 1 | atccttatcttatccccaca | cgg |
| 25284381 | −1 | ataacataacatgccgtgtg | ggg |
| 25284382 | −1 | gataacataacatgccgtgt | ggg |
| 25284383 | −1 | ggataacataacatgccgtg | tgg |
| 25284404 | −1 | atgttctcaactgaataatg | ggg |
| 25284405 | −1 | aatgttctcaactgaataat | ggg |
| 25284406 | −1 | caatgttctcaactgaataa | tgg |
| 25284420 | 1 | ttattcagttgagaacattg | agg |
| 25284430 | 1 | gagaacattgaggctcaaag | agg |
| 25284455 | −1 | CAAGATCGTTTACAAGTATt | tgg |
| 25284482 | −1 | TACTAAATGGCAGCTGGAAG | GGG |
| 25284483 | −1 | TTACTAAATGGCAGCTGGAA | GGG |
| 25284484 | −1 | CTTACTAAATGGCAGCTGGA | AGG |
| 25284488 | −1 | GAGTCTTACTAAATGGCAGC | TGG |
| 25284495 | −1 | GAAATTAGAGTCTTACTAAA | TGG |
| 25284521 | −1 | GAAGCAGACGAGATTTAGGG | TGG |
| 25284524 | −1 | GGGGAAGCAGACGAGATTTA | GGG |
| 25284525 | −1 | GGGGGAAGCAGACGAGATTT | AGG |
| 25284543 | −1 | AGATGGCGAGAAGGACGAGG | GGG |
| 25284544 | −1 | GAGATGGCGAGAAGGACGAG | GGG |
| 25284545 | −1 | GGAGATGGCGAGAAGGACGA | GGG |
| 25284546 | −1 | GGGAGATGGCGAGAAGGACG | AGG |
| 25284552 | −1 | TCGGTGGGGAGATGGCGAGA | AGG |
| 25284560 | −1 | CCAACTGCTCGGTGGGGAGA | TGG |
| 25284566 | −1 | TCTTGGCCAACTGCTCGGTG | GGG |
| 25284567 | −1 | ATCTTGGCCAACTGCTCGGT | GGG |
| 25284568 | −1 | GATCTTGGCCAACTGCTCGG | TGG |
| 25284571 | 1 | CCATCTCCCCACCGAGCAGT | TGG |
| 25284571 | −1 | TCAGATCTTGGCCAACTGCT | CGG |
| 25284583 | −1 | CCGCCATCACGGTCAGATCT | TGG |
| 25284591 | 1 | TGGCCAAGATCTGACCGTGA | TGG |
| 25284594 | 1 | CCAAGATCTGACCGTGATGG | CGG |
| 25284594 | −1 | CAAGCCAATGGCCGCCATCA | CGG |
| 25284601 | 1 | CTGACCGTGATGGCGGCCAT | TGG |
| 25284606 | 1 | CGTGATGGCGGCCATTGGCT | TGG |
| 25284606 | −1 | GGTGAGGAAGCCCAAGCCAA | TGG |
| 25284607 | 1 | GTGATGGCGGCCATTGGCTT | GGG |
| 25284622 | −1 | GTCTCCGGAAACTCGAGGTG | AGG |
| 25284627 | −1 | GCTGTGTCTCCGGAAACTCG | AGG |
| 25284629 | 1 | GCTTCCTCACCTCGAGTTTC | CGG |
| 25284637 | −1 | CACTGCTCCAGCTGTGTCTC | CGG |
| 25284641 | 1 | CGAGTTTCCGGAGACACAGC | TGG |
| 25284651 | 1 | GAGACACAGCTGGAGCAGTG | TGG |
| 25284663 | −1 | CGCCAGCATGAAGAGGTTGA | AGG |
| 25284670 | −1 | CACCAAGCGCCAGCATGAAG | AGG |
| 25284672 | 1 | GGCCTTCAACCTCTTCATGC | TGG |
| 25284679 | 1 | AACCTCTTCATGCTGGCGCT | TGG |
| 25284689 | 1 | TGCTGGCGCTTGGTGTGCAG | TGG |
| 25284690 | 1 | GCTGGCGCTTGGTGTGCAGT | GGG |
| 25284702 | 1 | TGTGCAGTGGGCAATCCTGC | TGG |
| 25284706 | 1 | CAGTGGGCAATCCTGCTGGA | CGG |
| 25284706 | −1 | GGCTCAGGAAGCCGTCCAGC | AGG |
| 25284721 | −1 | TCCCAGAAGGGAACTGGCTC | AGG |
| 25284727 | −1 | CCACCTTCCCAGAAGGGAAC | TGG |
| 25284730 | 1 | TTCCTGAGCCAGTTCCCTTC | TGG |
| 25284731 | 1 | TCCTGAGCCAGTTCCCTTCT | GGG |
| 25284733 | −1 | TGATGACCACCTTCCCAGAA | GGG |
| 25284734 | −1 | GTGATGACCACCTTCCCAGA | AGG |
| 25284735 | 1 | GAGCCAGTTCCCTTCTGGGA | AGG |
| 25284738 | 1 | CCAGTTCCCTTCTGGGAAGG | TGG |
| 25284755 | 1 | AGGTGGTCATCACACTGTTC | AGG |
| 25284761 | 1 | TCATCACACTGTTCAGGTAT | TGG |
| 25284762 | 1 | CATCACACTGTTCAGGTATT | GGG |
| 25284766 | 1 | ACACTGTTCAGGTATTGGGA | TGG |
| 25284769 | 1 | CTGTTCAGGTATTGGGATGG | TGG |
| 25284773 | 1 | TCAGGTATTGGGATGGTGGC | TGG |
| 25284784 | 1 | GATGGTGGCTGGATCACTTC | TGG |
| 25284785 | 1 | ATGGTGGCTGGATCACTTCT | GGG |
| 25284794 | 1 | GGATCACTTCTGGGTCATAG | AGG |
| 25284795 | 1 | GATCACTTCTGGGTCATAGA | GGG |
| 25284800 | 1 | CTTCTGGGTCATAGAGGGAA | TGG |
| 25284811 | 1 | TAGAGGGAATGGACCCCGAA | AGG |
| 25284813 | −1 | TTCTGGAACCTGTCCTTTCG | GGG |
| 25284814 | −1 | CTTCTGGAACCTGTCCTTTC | GGG |
| 25284815 | −1 | TCTTCTGGAACCTGTCCTTT | CGG |
| 25284816 | 1 | GGAATGGACCCCGAAAGGAC | AGG |
| 25284830 | −1 | GGGCAATATCCCAGATCTTC | TGG |
| 25284831 | 1 | AGGACAGGTTCCAGAAGATC | TGG |
| 25284832 | 1 | GGACAGGTTCCAGAAGATCT | GGG |
| 25284850 | −1 | acTGGTGCTAGACAGAGAGG | GGG |
| 25284851 | −1 | cacTGGTGCTAGACAGAGAG | GGG |
| 25284852 | −1 | gcacTGGTGCTAGACAGAGA | GGG |
| 25284853 | −1 | agcacTGGTGCTAGACAGAG | AGG |
| 25284868 | −1 | tcctaaatattgcacagcac | TGG |
| 25284878 | 1 | ACCAgtgctgtgcaatattt | agg |
| 25284894 | −1 | atgaataatcttttagtata | agg |
| 25284928 | 1 | tgtttaaaattcaaattaac | tgg |
| 25284929 | 1 | gtttaaaattcaaattaact | ggg |
| 25284944 | −1 | agggctgtccagtaaaatac | agg |
| 25284947 | 1 | ctgggcatcctgtattttac | tgg |
| 25284963 | −1 | TCCTTGTGATACACGGAGta | ggg |
| 25284964 | −1 | TTCCTTGTGATACACGGAGt | agg |
| 25284970 | −1 | CCTGGATTCCTTGTGATACA | CGG |
| 25284973 | 1 | gccctaCTCCGTGTATCACA | AGG |
| 25284981 | 1 | CCGTGTATCACAAGGAATCC | AGG |
| 25284988 | −1 | ATGCAGGAGGAATGTAGGCC | TGG |
| 25284993 | −1 | AAAGGATGCAGGAGGAATGT | AGG |
| 25285001 | −1 | CAGGAAAGAAAGGATGCAGG | AGG |
| 25285004 | −1 | TAACAGGAAAGAAAGGATGC | AGG |
| 25285011 | −1 | TCGACAATAACAGGAAAGAA | AGG |
| 25285020 | −1 | AAATCATAATCGACAATAAC | AGG |
| 25285063 | 1 | ACATAATCAATATAAGTTTA | TGG |
| 25285077 | 1 | AGTTTATGGAAAACGTAAGA | AGG |
| 25285119 | −1 | atagaaTGTCTCTCTAGGTG | TGG |
| 25285124 | −1 | aaaaaatagaaTGTCTCTCT | AGG |
| 25285153 | 1 | ttttttttttttttttgaga | cgg |
| 25285175 | 1 | gagtttcacttttgttgccc | agg |
| 25285179 | 1 | ttcacttttgttgcccaggc | tgg |
| 25285181 | −1 | gcgccattgcactccagcct | ggg |
| 25285182 | −1 | agcgccattgcactccagcc | tgg |
| 25285189 | 1 | ttgcccaggctggagtgcaa | tgg |
| 25285200 | 1 | ggagtgcaatggcgctatct | cgg |
| 25285216 | −1 | aacccagaaggctgaggttg | tgg |
| 25285222 | −1 | cgcttgaacccagaaggctg | agg |
| 25285224 | 1 | acaccacaacctcagccttc | tgg |
| 25285225 | 1 | caccacaacctcagccttct | ggg |
| 25285228 | −1 | gagaatcgcttgaacccaga | agg |
| 25285250 | −1 | gctactcaggcggctgaggc | agg |
| 25285254 | −1 | cccagctactcaggcggctg | agg |
| 25285260 | −1 | tgtaatcccagctactcagg | cgg |
| 25285263 | −1 | gcctgtaatcccagctactc | agg |
| 25285264 | 1 | gcctcagccgcctgagtagc | tgg |
| 25285265 | 1 | cctcagccgcctgagtagct | ggg |
| 25285273 | 1 | gcctgagtagctgggattac | agg |
| 25285291 | −1 | acaaaatcagccaggcgcgg | tgg |
| 25285292 | 1 | caggcatgtgccaccgcgcc | tgg |
| 25285294 | −1 | aatacaaaatcagccaggcg | cgg |
| 25285299 | −1 | ctaaaaatacaaaatcagcc | agg |
| 25285320 | 1 | tttgtatttttagtagagat | agg |
| 25285321 | 1 | ttgtatttttagtagagata | ggg |
| 25285335 | 1 | gagatagggtttctccgtgt | tgg |
| 25285338 | −1 | tgagactagcctgaccaaca | cgg |
| 25285340 | 1 | agggtttctccgtgttggtc | agg |
| 25285365 | 1 | agtctcaaactcctgacctc | agg |
| 25285365 | −1 | cgggcggatcacctgaggtc | agg |
| 25285370 | −1 | cgaggcgggcggatcacctg | agg |
| 25285381 | −1 | ctttgggaggccgaggcggg | cgg |
| 25285382 | 1 | ctcaggtgatccgcccgcct | cgg |
| 25285384 | −1 | gcactttgggaggccgaggc | ggg |
| 25285385 | −1 | agcactttgggaggccgagg | cgg |
| 25285388 | −1 | cccagcactttgggaggccg | agg |
| 25285394 | −1 | tgtaatcccagcactttggg | agg |
| 25285397 | −1 | gtctgtaatcccagcacttt | ggg |
| 25285398 | 1 | gcctcggcctcccaaagtgc | tgg |
| 25285398 | −1 | tgtctgtaatcccagcactt | tgg |
| 25285399 | 1 | cctcggcctcccaaagtgct | ggg |
| 25285425 | −1 | GTCTCTCAggctggacgcgg | tgg |
| 25285428 | −1 | AATGTCTCTCAggctggacg | cgg |
| 25285434 | −1 | CAAGAGAATGTCTCTCAggc | tgg |
| 25285438 | −1 | TTTTCAAGAGAATGTCTCTC | Agg |
| 25285455 | 1 | AGACATTCTCTTGAAAAGAA | AGG |
| 25285475 | −1 | TATTGTCTAGCAGCATTAGG | GGG |
| 25285476 | −1 | TTATTGTCTAGCAGCATTAG | GGG |
| 25285477 | −1 | TTTATTGTCTAGCAGCATTA | GGG |
| 25285478 | −1 | ATTTATTGTCTAGCAGCATT | AGG |
| 25285503 | −1 | ATTTAATGAAAATAAAGGCA | TGG |
| 25285508 | −1 | AGGTAATTTAATGAAAATAA | AGG |
| 25285528 | −1 | aatgCATGTAAACAAAGCAC | AGG |
| 25285569 | −1 | gcaccatacattagttgtga | tgg |
| 25285577 | 1 | gaaccatcacaactaatgta | tgg |
| 25285591 | −1 | gtaacaactattctgacttc | tgg |
| 25285606 | 1 | aagtcagaatagttgttacc | tgg |
| 25285607 | 1 | agtcagaatagttgttacct | ggg |
| 25285611 | 1 | agaatagttgttacctgggc | agg |
| 25285613 | −1 | tcaatatccacctcctgccc | agg |
| 25285614 | 1 | atagttgttacctgggcagg | agg |
| 25285617 | 1 | gttgttacctgggcaggagg | tgg |
| 25285629 | 1 | gcaggaggtggatattgatt | agg |
| 25285633 | 1 | gaggtggatattgattagga | agg |
| 25285653 | 1 | aggaacacaaaataaccgca | tgg |
| 25285654 | 1 | ggaacacaaaataaccgcat | ggg |
| 25285655 | 1 | gaacacaaaataaccgcatg | ggg |
| 25285657 | −1 | aacattttctgcaccccatg | cgg |
| 25285684 | 1 | aaatgttctctatgttcacc | tgg |
| 25285685 | 1 | aatgttctctatgttcacct | ggg |
| 25285691 | −1 | ttgatgtgtaatcatcaccc | agg |
| 25285722 | 1 | caagctatacacgttttaaa | aGG |
| 25285723 | 1 | aagctatacacgttttaaaa | GGG |
| 25285729 | 1 | tacacgttttaaaaGGGCAT | TGG |
| 25285740 | 1 | aaaGGGCATTGGCACTTAAT | AGG |
| 25285743 | 1 | GGGCATTGGCACTTAATAGG | AGG |
| 25285750 | 1 | GGCACTTAATAGGAGGAAGT | AGG |
| 25285773 | −1 | ACAAAACAAAACAATGTTTC | AGG |
| 25285801 | −1 | TGGGCAGCACAGGGATTCAG | AGG |
| 25285810 | −1 | ACCATCATCTGGGCAGCACA | GGG |
| 25285811 | −1 | TACCATCATCTGGGCAGCAC | AGG |
| 25285820 | 1 | TCCCTGTGCTGCCCAGATGA | TGG |
| 25285820 | −1 | GATGACGTTTACCATCATCT | GGG |
| 25285821 | −1 | GGATGACGTTTACCATCATC | TGG |
| 25285836 | 1 | ATGATGGTAAACGTCATCCT | AGG |
| 25285842 | −1 | GAGAGGTCCCTAAGATGCCT | AGG |
| 25285845 | 1 | AACGTCATCCTAGGCATCTT | AGG |
| 25285846 | 1 | ACGTCATCCTAGGCATCTTA | GGG |
| 25285857 | 1 | GGCATCTTAGGGACCTCTCA | AGG |
| 25285859 | −1 | GAGGCTGGAATGGCCTTGAG | AGG |
| 25285869 | −1 | CTTAGAAGGGGAGGCTGGAA | TGG |
| 25285874 | −1 | AGGGTCTTAGAAGGGGAGGC | TGG |
| 25285878 | −1 | TAGCAGGGTCTTAGAAGGGG | AGG |
| 25285881 | −1 | GTTTAGCAGGGTCTTAGAAG | GGG |
| 25285882 | −1 | GGTTTAGCAGGGTCTTAGAA | GGG |
| 25285883 | −1 | AGGTTTAGCAGGGTCTTAGA | AGG |
| 25285893 | −1 | CAGTGCCCAGAGGTTTAGCA | GGG |
| 25285894 | −1 | GCAGTGCCCAGAGGTTTAGC | AGG |
| 25285898 | 1 | CTAAGACCCTGCTAAACCTC | TGG |
| 25285899 | 1 | TAAGACCCTGCTAAACCTCT | GGG |
| 25285903 | −1 | tgtttaacaGCAGTGCCCAG | AGG |
| 25285931 | 1 | taaacatttctctatgagcc | agg |
| 25285938 | −1 | ggagtgctcagcacagttcc | tgg |
| 25285959 | −1 | gttaaacaaaataatatttg | tgg |
| 25285978 | 1 | attattttgtttaactcttc | cgg |
| 25285979 | 1 | ttattttgtttaactcttcc | ggg |
| 25285983 | 1 | tttgtttaactcttccgggt | agg |
| 25285984 | 1 | ttgtttaactcttccgggta | ggg |
| 25285986 | −1 | taccaggttagatccctacc | cgg |
| 25285995 | 1 | ttccgggtagggatctaacc | tgg |
| 25286002 | −1 | cacttccttacctgtatacc | agg |
| 25286003 | 1 | agggatctaacctggtatac | agg |
| 25286008 | 1 | tctaacctggtatacaggta | agg |
| 25286014 | 1 | ctggtatacaggtaaggaag | tgg |
| 25286027 | 1 | aaggaagtggaagctcagag | agg |
| 25286028 | 1 | aggaagtggaagctcagaga | ggg |
| 25286033 | 1 | gtggaagctcagagagggca | agg |
| 25286045 | 1 | agagggcaaggcacttgcct | agg |
| 25286046 | 1 | gagggcaaggcacttgccta | ggg |
| 25286051 | −1 | ccacttagctgtgtggccct | agg |
| 25286058 | −1 | ATctccaccacttagctgtg | tgg |
| 25286062 | 1 | cctagggccacacagctaag | tgg |
| 25286065 | 1 | agggccacacagctaagtgg | tgg |
| 25286071 | 1 | acacagctaagtggtggagA | TGG |
| 25286085 | −1 | AAAAGGTTATAATAAAAAGT | TGG |
| 25286102 | −1 | CACTCTGGAGCATGTGGAAA | AGG |
| 25286108 | −1 | TCTGAGCACTCTGGAGCATG | TGG |
| 25286117 | −1 | GTTTCATGTTCTGAGCACTC | TGG |
| 25286149 | −1 | CTCCAGGGCCAATCGGGAGC | TGG |
| 25286152 | 1 | CAGTCTAGCCAGCTCCCGAT | TGG |
| 25286155 | −1 | TTTTCCCTCCAGGGCCAATC | GGG |
| 25286156 | −1 | TTTTTCCCTCCAGGGCCAAT | CGG |
| 25286158 | 1 | AGCCAGCTCCCGATTGGCCC | TGG |
| 25286161 | 1 | CAGCTCCCGATTGGCCCTGG | AGG |
| 25286162 | 1 | AGCTCCCGATTGGCCCTGGA | GGG |
| 25286164 | −1 | TATAAAGTTTTTTCCCTCCA | GGG |
| 25286165 | −1 | ATATAAAGTTTTTTCCCTCC | AGG |
| 25286195 | 1 | ATATATTTTTCTTTTTTAAA | AGG |
| 25286203 | 1 | TTCTTTTTTAAAAGGTTTAG | Agg |
| 25286207 | 1 | TTTTTAAAAGGTTTAGAggc | tgg |
| 25286208 | 1 | TTTTAAAAGGTTTAGAggct | ggg |
| 25286213 | 1 | AAAGGTTTAGAggctgggca | tgg |
| 25286216 | 1 | GGTTTAGAggctgggcatgg | tgg |
| 25286234 | −1 | cccaaaagtactgggattac | agg |
| 25286242 | −1 | cctcggttcccaaaagtact | ggg |
| 25286243 | −1 | acctcggttcccaaaagtac | tgg |
| 25286244 | 1 | acctgtaatcccagtacttt | tgg |
| 25286245 | 1 | cctgtaatcccagtactttt | ggg |
| 25286253 | 1 | cccagtacttttgggaaccg | agg |
| 25286256 | 1 | agtacttttgggaaccgagg | tgg |
| 25286257 | 1 | gtacttttgggaaccgaggt | ggg |
| 25286259 | −1 | ctcaagtgatctgcccacct | cgg |
| 25286282 | −1 | caggctggtcttaaacttct | ggg |
| 25286283 | −1 | tcaggctggtcttaaacttc | tgg |
| 25286297 | −1 | tctcactgtgttagtcaggc | tgg |
| 25286301 | −1 | aggatctcactgtgttagtc | agg |
| 25286321 | −1 | tttctattttctgcagagac | agg |
| 25286343 | 1 | agaaaatagaaaaatcagct | agg |
| 25286348 | 1 | atagaaaaatcagctaggcg | tgg |
| 25286351 | 1 | gaaaaatcagctaggcgtgg | tgg |
| 25286369 | −1 | tcccaagtagctgggactgt | ggg |
| 25286370 | −1 | ctcccaagtagctgggactg | tgg |
| 25286377 | −1 | cctcagcctcccaagtagct | ggg |
| 25286378 | 1 | cacccacagtcccagctact | tgg |
| 25286378 | −1 | gcctcagcctoccaagtagc | tgg |
| 25286379 | 1 | acccacagtcccagctactt | ggg |
| 25286382 | 1 | cacagtcccagctacttggg | agg |
| 25286388 | 1 | cccagctacttgggaggctg | agg |
| 25286392 | 1 | gctacttgggaggctgaggc | agg |
| 25286395 | 1 | acttgggaggctgaggcagg | agg |
| 25286411 | −1 | gcctcaacctcactgggttc | agg |
| 25286415 | 1 | aggatcacctgaacccagtg | agg |
| 25286417 | −1 | cactcagcctcaacctcact | ggg |
| 25286418 | −1 | tcactcagcctcaacctcac | tgg |
| 25286421 | 1 | acctgaacccagtgaggttg | agg |
| 25286442 | −1 | ggagtgaagtggcacgatca | tgg |
| 25286453 | −1 | ttgtccaggctggagtgaag | tgg |
| 25286460 | 1 | cgtgccacttcactccagcc | tgg |
| 25286463 | −1 | tctcactctgttgtccaggc | tgg |
| 25286467 | −1 | agggtctcactctgttgtcc | agg |
| 25286486 | −1 | taaaactgttttttgagaca | ggg |
| 25286487 | −1 | ctaaaactgttttttgagac | agg |
| 25286499 | 1 | ctgtctcaaaaaacagtttt | agg |
| 25286500 | 1 | tgtctcaaaaaacagtttta | ggg |
| 25286501 | 1 | gtctcaaaaaacagttttag | ggg |
| 25286505 | 1 | caaaaaacagttttaggggc | cgg |
| 25286506 | 1 | aaaaaacagttttaggggcc | ggg |
| 25286513 | −1 | caggcatgaaccactgcgcc | cgg |
| 25286514 | 1 | gttttaggggccgggcgcag | tgg |
| 25286532 | −1 | tcccaaagtgctgggattac | agg |
| 25286540 | −1 | ccttggcctcccaaagtgct | ggg |
| 25286541 | 1 | tgcctgtaatcccagcactt | tgg |
| 25286541 | −1 | gccttggcctcccaaagtgc | tgg |
| 25286542 | 1 | gcctgtaatcccagcacttt | ggg |
| 25286545 | 1 | tgtaatcccagcactttggg | agg |
| 25286551 | 1 | cccagcactttgggaggcca | agg |
| 25286554 | 1 | agcactttgggaggccaagg | cgg |
| 25286555 | 1 | gcactttgggaggccaaggc | ggg |
| 25286556 | 1 | cactttgggaggccaaggcg | ggg |
| 25286557 | 1 | actttgggaggccaaggcgg | ggg |
| 25286557 | −1 | acctcatgatccccccgcct | tgg |
| 25286558 | 1 | ctttgggaggccaaggcggg | ggg |
| 25286567 | 1 | gccaaggcggggggatcatg | agg |
| 25286572 | 1 | ggcggggggatcatgaggtc | agg |
| 25286590 | 1 | tcaggagatcgagaccatcc | tgg |
| 25286593 | −1 | tttctccgagttagccagga | tgg |
| 25286597 | −1 | agggtttctccgagttagcc | agg |
| 25286599 | 1 | cgagaccatcctggctaact | cgg |
| 25286616 | −1 | ttgtatttttagtagagaca | ggg |
| 25286617 | −1 | tttgtatttttagtagagac | agg |
| 25286639 | 1 | taaaaatacaaaaaattagc | cgg |
| 25286640 | 1 | aaaaatacaaaaaattagcc | ggg |
| 25286645 | 1 | tacaaaaaattagccgggcg | tgg |
| 25286647 | −1 | caggcgcccaccaccacgcc | cgg |
| 25286648 | 1 | aaaaaattagccgggcgtgg | tgg |
| 25286651 | 1 | aaattagccgggcgtggtgg | tgg |
| 25286652 | 1 | aattagccgggcgtggtggt | ggg |
| 25286666 | −1 | tcccgagtggctgggactac | agg |
| 25286674 | −1 | cctcagcctcccgagtggct | ggg |
| 25286675 | 1 | cgcctgtagtcccagccact | cgg |
| 25286675 | −1 | gcctcagcctcccgagtggc | tgg |
| 25286676 | 1 | gcctgtagtcccagccactc | ggg |
| 25286679 | 1 | tgtagtcccagccactcggg | agg |
| 25286679 | −1 | tcctgcctcagcctcccgag | tgg |
| 25286685 | 1 | cccagccactcgggaggctg | agg |
| 25286689 | 1 | gccactcgggaggctgaggc | agg |
| 25286696 | 1 | gggaggctgaggcaggagaa | tgg |
| 25286707 | 1 | gcaggagaatggcgtgaacc | cgg |
| 25286708 | 1 | caggagaatggcgtgaaccc | ggg |
| 25286711 | 1 | gagaatggcgtgaacccggg | agg |
| 25286714 | 1 | aatggcgtgaacccgggagg | cgg |
| 25286714 | −1 | cactgcaaactccgcctccc | ggg |
| 25286715 | −1 | tcactgcaaactccgcctcc | cgg |
| 25286736 | 1 | gagtttgcagtgaaccgaga | tgg |
| 25286739 | −1 | ggagtgcagtggcaccatct | cgg |
| 25286750 | −1 | tcacccaggctggagtgcag | tgg |
| 25286757 | 1 | ggtgccactgcactccagcc | tgg |
| 25286758 | 1 | gtgccactgcactccagcct | ggg |
| 25286760 | −1 | tctcgctctgtcacccaggc | tgg |
| 25286764 | −1 | ggagtctcgctctgtcaccc | agg |
| 25286785 | −1 | tttgttttttttttttgaga | cgg |
| 25286808 | 1 | aaaaaaacaaaaacagtttt | agg |
| 25286813 | 1 | aacaaaaacagttttaggcc | agg |
| 25286818 | 1 | aaacagttttaggccaggcg | cgg |
| 25286820 | −1 | caggcatgaaccaccgcgcc | tgg |
| 25286821 | 1 | cagttttaggccaggcgcgg | tgg |
| 25286839 | −1 | tcctaaagtactaggattac | agg |
| 25286847 | −1 | gctaggcctcctaaagtact | agg |
| 25286849 | 1 | gcctgtaatcctagtacttt | agg |
| 25286852 | 1 | tgtaatcctagtactttagg | agg |
| 25286861 | 1 | agtactttaggaggcctagc | agg |
| 25286864 | 1 | actttaggaggcctagcagg | tgg |
| 25286864 | −1 | cctcaggtaatccacctgct | agg |
| 25286875 | 1 | cctagcaggtggattacctg | agg |
| 25286880 | 1 | caggtggattacctgaggtc | agg |
| 25286880 | −1 | ggtctcggactcctgacctc | agg |
| 25286895 | −1 | catgttgctcaggttggtct | cgg |
| 25286901 | −1 | tttcaccatgttgctcaggt | tgg |
| 25286905 | −1 | aggatttcaccatgttgctc | agg |
| 25286907 | 1 | cgagaccaacctgagcaaca | tgg |
| 25286925 | −1 | tttgtgtttttagtagagac | agg |
| 25286946 | 1 | ctaaaaacacaaaaattagc | tgg |
| 25286947 | 1 | taaaaacacaaaaattagct | ggg |
| 25286952 | 1 | acacaaaaattagctgggtg | tgg |
| 25286955 | 1 | caaaaattagctgggtgtgg | cgg |
| 25286959 | 1 | aattagctgggtgtggcggc | agg |
| 25286973 | −1 | tcccaagtagctgggattac | agg |
| 25286981 | −1 | cctcagcctcccaagtagct | ggg |
| 25286982 | 1 | cacctgtaatcccagctact | tgg |
| 25286982 | −1 | gcctcagcctoccaagtagc | tgg |
| 25286983 | 1 | acctgtaatcccagctactt | ggg |
| 25286986 | 1 | tgtaatcccagctacttggg | agg |
| 25286992 | 1 | cccagctacttgggaggctg | agg |
| 25286996 | 1 | gctacttgggaggctgaggc | agg |
| 25287014 | 1 | gcaggcgaatcacttgaacc | cgg |
| 25287015 | 1 | caggcgaatcacttgaaccc | ggg |
| 25287018 | 1 | gcgaatcacttgaacccggg | agg |
| 25287021 | 1 | aatcacttgaacccgggagg | cgg |
| 25287021 | −1 | cactatagcctccgcctccc | ggg |
| 25287022 | −1 | tcactatagcctccgcctcc | cgg |
| 25287024 | 1 | cacttgaacccgggaggcgg | agg |
| 25287046 | −1 | acagtgcaatggtgcgatct | cgg |
| 25287057 | −1 | tcgcccaggctacagtgcaa | tgg |
| 25287064 | 1 | cgcaccattgcactgtagcc | tgg |
| 25287065 | 1 | gcaccattgcactgtagcct | ggg |
| 25287071 | −1 | agagcctcactctgtcgccc | agg |
| 25287078 | 1 | gtagcctgggcgacagagtg | agg |
| 25287137 | −1 | tgtgtgtaTTGAATTCTGGT | GGG |
| 25287138 | −1 | gtgtgtgtaTTGAATTCTGG | TGG |
| 25287141 | −1 | tgcgtgtgtgtaTTGAATTC | TGG |
| 25287186 | 1 | atacacacacTGTGTCCACC | TGG |
| 25287187 | 1 | tacacacacTGTGTCCACCT | GGG |
| 25287190 | −1 | GCCCTTTGTCACTTCCCAGG | TGG |
| 25287193 | −1 | GGTGCCCTTTGTCACTTCCC | AGG |
| 25287199 | 1 | GTCCACCTGGGAAGTGACAA | AGG |
| 25287200 | 1 | TCCACCTGGGAAGTGACAAA | GGG |
| 25287208 | 1 | GGAAGTGACAAAGGGCACCC | TGG |
| 25287209 | 1 | GAAGTGACAAAGGGCACCCT | GGG |
| 25287210 | 1 | AAGTGACAAAGGGCACCCTG | GGG |
| 25287211 | 1 | AGTGACAAAGGGCACCCTGG | GGG |
| 25287214 | −1 | CCACCATTTGAAATCCCCCA | GGG |
| 25287215 | −1 | ACCACCATTTGAAATCCCCC | AGG |
| 25287222 | 1 | GCACCCTGGGGGATTTCAAA | TGG |
| 25287225 | 1 | CCCTGGGGGATTTCAAATGG | TGG |
| 25287228 | 1 | TGGGGGATTTCAAATGGTGG | TGG |
| 25287234 | 1 | ATTTCAAATGGTGGTGGCCC | TGG |
| 25287239 | 1 | AAATGGTGGTGGCCCTGGTT | TGG |
| 25287240 | −1 | AAGGCAGCAACACCAAACCA | GGG |
| 25287241 | −1 | TAAGGCAGCAACACCAAACC | AGG |
| 25287259 | −1 | GCTGGTGTGACCTTAAGCTA | AGG |
| 25287260 | 1 | GGTGTTGCTGCCTTAGCTTA | AGG |
| 25287277 | −1 | TGGGGCAGGAGGCTGAAGGC | TGG |
| 25287281 | −1 | ACTGTGGGGCAGGAGGCTGA | AGG |
| 25287288 | −1 | GCCCTAGACTGTGGGGCAGG | AGG |
| 25287291 | −1 | GCAGCCCTAGACTGTGGGGC | AGG |
| 25287295 | −1 | GGGAGCAGCCCTAGACTGTG | GGG |
| 25287296 | −1 | GGGGAGCAGCCCTAGACTGT | GGG |
| 25287297 | 1 | AGCCTCCTGCCCCACAGTCT | AGG |
| 25287297 | −1 | AGGGGAGCAGCCCTAGACTG | TGG |
| 25287298 | 1 | GCCTCCTGCCCCACAGTCTA | GGG |
| 25287315 | −1 | CCCTGTGGACATCAGATGAG | GGG |
| 25287316 | −1 | TCCCTGTGGACATCAGATGA | GGG |
| 25287317 | −1 | GTCCCTGTGGACATCAGATG | AGG |
| 25287325 | 1 | TCCCCTCATCTGATGTCCAC | AGG |
| 25287326 | 1 | CCCCTCATCTGATGTCCACA | GGG |
| 25287330 | −1 | CAAGAACAAACAGGTCCCTG | TGG |
| 25287339 | −1 | AGATTGAGTCAAGAACAAAC | AGG |
| 25287365 | 1 | CTCAATCTAGAAAGACGAGA | AGG |
| 25287366 | 1 | TCAATCTAGAAAGACGAGAA | GGG |
| 25287407 | −1 | AGCAGTCAGGGGTGGGGCAG | GGG |
| 25287408 | −1 | AAGCAGTCAGGGGTGGGGCA | GGG |
| 25287409 | −1 | CAAGCAGTCAGGGGTGGGGC | AGG |
| 25287413 | −1 | GATCCAAGCAGTCAGGGGTG | GGG |
| 25287414 | −1 | GGATCCAAGCAGTCAGGGGT | GGG |
| 25287415 | −1 | GGGATCCAAGCAGTCAGGGG | TGG |
| 25287418 | −1 | AGGGGGATCCAAGCAGTCAG | GGG |
| 25287419 | −1 | TAGGGGGATCCAAGCAGTCA | GGG |
| 25287420 | −1 | CTAGGGGGATCCAAGCAGTC | AGG |
| 25287421 | 1 | CTGCCCCACCCCTGACTGCT | TGG |
| 25287432 | 1 | CTGACTGCTTGGATCCCCCT | AGG |
| 25287433 | 1 | TGACTGCTTGGATCCCCCTA | GGG |
| 25287434 | 1 | GACTGCTTGGATCCCCCTAG | GGG |
| 25287435 | −1 | CAGCAGGGGTCACCCCTAGG | GGG |
| 25287436 | −1 | TCAGCAGGGGTCACCCCTAG | GGG |
| 25287437 | −1 | TTCAGCAGGGGTCACCCCTA | GGG |
| 25287438 | −1 | TTTCAGCAGGGGTCACCCCT | AGG |
| 25287449 | −1 | GAAGGAGCCAGTTTCAGCAG | GGG |
| 25287450 | −1 | GGAAGGAGCCAGTTTCAGCA | GGG |
| 25287451 | −1 | AGGAAGGAGCCAGTTTCAGC | AGG |
| 25287453 | 1 | GGGGTGACCCCTGCTGAAAC | TGG |
| 25287467 | −1 | CTGACGGGAACCGGTCAGGA | AGG |
| 25287468 | 1 | GAAACTGGCTCCTTCCTGAC | CGG |
| 25287471 | −1 | AGCCCTGACGGGAACCGGTC | AGG |
| 25287476 | −1 | AGCACAGCCCTGACGGGAAC | CGG |
| 25287479 | 1 | CTTCCTGACCGGTTCCCGTC | AGG |
| 25287480 | 1 | TTCCTGACCGGTTCCCGTCA | GGG |
| 25287482 | −1 | CCCATCAGCACAGCCCTGAC | GGG |
| 25287483 | −1 | ACCCATCAGCACAGCCCTGA | CGG |
| 25287492 | 1 | TCCCGTCAGGGCTGTGCTGA | TGG |
| 25287493 | 1 | CCCGTCAGGGCTGTGCTGAT | GGG |
| 25287496 | 1 | GTCAGGGCTGTGCTGATGGG | TGG |
| 25287504 | 1 | TGTGCTGATGGGTGGTGCCC | AGG |
| 25287510 | −1 | CCGTCCCCAGGGGCAGGCCT | GGG |
| 25287511 | −1 | CCCGTCCCCAGGGGCAGGCC | TGG |
| 25287515 | 1 | GTGGTGCCCAGGCCTGCCCC | TGG |
| 25287516 | 1 | TGGTGCCCAGGCCTGCCCCT | GGG |
| 25287516 | −1 | AGTACCCCGTCCCCAGGGGC | AGG |
| 25287517 | 1 | GGTGCCCAGGCCTGCCCCTG | GGG |
| 25287520 | −1 | GGAGAGTACCCCGTCCCCAG | GGG |
| 25287521 | 1 | CCCAGGCCTGCCCCTGGGGA | CGG |
| 25287521 | −1 | GGGAGAGTACCCCGTCCCCA | GGG |
| 25287522 | 1 | CCAGGCCTGCCCCTGGGGAC | GGG |
| 25287522 | −1 | AGGGAGAGTACCCCGTCCCC | AGG |
| 25287523 | 1 | CAGGCCTGCCCCTGGGGACG | GGG |
| 25287536 | 1 | GGGGACGGGGTACTCTCCCT | TGG |
| 25287541 | −1 | ACAAGCTGGAGTGTTGCCAA | GGG |
| 25287542 | −1 | CACAAGCTGGAGTGTTGCCA | AGG |
| 25287555 | −1 | CCAAGTCAAGTGGCACAAGC | TGG |
| 25287565 | −1 | CAAATCAGTCCCAAGTCAAG | TGG |
| 25287566 | 1 | CCAGCTTGTGCCACTTGACT | TGG |
| 25287567 | 1 | CAGCTTGTGCCACTTGACTT | GGG |
| 25287577 | 1 | CACTTGACTTGGGACTGATT | TGG |
| 25287599 | 1 | GTTCTGTTTtgagtcccttc | agg |
| 25287600 | 1 | TTCTGTTTtgagtcccttca | ggg |
| 25287601 | 1 | TCTGTTTtgagtcccttcag | ggg |
| 25287602 | −1 | agataggcccctcccctgaa | ggg |
| 25287603 | −1 | aagataggcccctcccctga | agg |
| 25287604 | 1 | GTTTtgagtcccttcagggg | agg |
| 25287605 | 1 | TTTtgagtcccttcagggga | ggg |
| 25287606 | 1 | TTtgagtcccttcaggggag | ggg |
| 25287618 | −1 | ACAacaacgttgaataagat | agg |
| 25287648 | −1 | TGCTAAGTTATCAGTATGTG | AGG |
| 25287664 | 1 | CATACTGATAACTTAGCAAA | TGG |
| 25287671 | 1 | ATAACTTAGCAAATGGCTAT | TGG |
| 25287692 | 1 | GGAGCAAAAATGAAAATAAA | CGG |
| 25287705 | 1 | AAATAAACGGAACTCTGAAG | TGG |
| 25287706 | 1 | AATAAACGGAACTCTGAAGT | GGG |
| 25287742 | 1 | ttatttatttttttagagac | agg |
| 25287743 | 1 | tatttatttttttagagaca | ggg |
| 25287766 | 1 | tcttgctctgttgcccagtc | tgg |
| 25287768 | −1 | gtaccactgcactccagact | ggg |
| 25287769 | −1 | tgtaccactgcactccagac | tgg |
| 25287776 | 1 | ttgcccagtctggagtgcag | tgg |
| 25287809 | −1 | cacttgagcccaggaggcac | agg |
| 25287811 | 1 | tcattgcagcctgtgcctcc | tgg |
| 25287812 | 1 | cattgcagcctgtgcctcct | ggg |
| 25287815 | −1 | gaggatcacttgagcccagg | agg |
| 25287818 | −1 | tgggaggatcacttgagccc | agg |
| 25287834 | −1 | actcaggaggctgaggtggg | agg |
| 25287837 | −1 | ttaactcaggaggctgaggt | ggg |
| 25287838 | −1 | tttaactcaggaggctgagg | tgg |
| 25287841 | −1 | aaatttaactcaggaggctg | agg |
| 25287847 | −1 | gtaaaaaaatttaactcagg | agg |
| 25287850 | −1 | cctgtaaaaaaatttaactc | agg |
| 25287861 | 1 | cctgagttaaatttttttac | agg |
| 25287875 | −1 | aattagcagggcatggtagc | agg |
| 25287882 | −1 | atacaaaaattagcagggca | tgg |
| 25287887 | −1 | taaaaatacaaaaattagca | ggg |
| 25287888 | −1 | ctaaaaatacaaaaattagc | agg |
| 25287908 | 1 | ttttgtatttttagtagaca | agg |
| 25287909 | 1 | tttgtatttttagtagacaa | ggg |
| 25287910 | 1 | ttgtatttttagtagacaag | ggg |
| 25287920 | 1 | agtagacaaggggtttcacc | agg |
| 25287923 | 1 | agacaaggggtttcaccagg | tgg |
| 25287924 | 1 | gacaaggggtttcaccaggt | ggg |
| 25287927 | −1 | ccagaccaacctgacccacc | tgg |
| 25287929 | 1 | ggggtttcaccaggtgggtc | agg |
| 25287933 | 1 | tttcaccaggtgggtcaggt | tgg |
| 25287938 | 1 | ccaggtgggtcaggttggtc | tgg |
| 25287954 | −1 | caggtggatcacttgaggtc | ggg |
| 25287955 | −1 | gcaggtggatcacttgaggt | cgg |
| 25287959 | −1 | ctaggcaggtggatcacttg | agg |
| 25287970 | −1 | ctttgggaggcctaggcagg | tgg |
| 25287971 | 1 | ctcaagtgatccacctgcct | agg |
| 25287973 | −1 | gtactttgggaggcctaggc | agg |
| 25287977 | −1 | cccagtactttgggaggcct | agg |
| 25287983 | −1 | tgtaatcccagtactttggg | agg |
| 25287986 | −1 | gcctgtaatcccagtacttt | ggg |
| 25287987 | 1 | gcctaggcctcccaaagtac | tgg |
| 25287987 | −1 | cgcctgtaatcccagtactt | tgg |
| 25287988 | 1 | cctaggcctcccaaagtact | ggg |
| 25287996 | 1 | tcccaaagtactgggattac | agg |
| 25288014 | −1 | CAGTTTTAggctggacacag | tgg |
| 25288023 | −1 | tctcaaaaaCAGTTTTAggc | tgg |
| 25288027 | −1 | cctgtctcaaaaaCAGTTTT | Agg |
| 25288038 | 1 | ccTAAAACTGtttttgagac | agg |
| 25288039 | 1 | cTAAAACTGtttttgagaca | ggg |
| 25288058 | 1 | agggtctcactctgttgtcc | agg |
| 25288062 | 1 | tctcactctgttgtccaggc | tgg |
| 25288065 | −1 | catgccacttcactccagcc | tgg |
| 25288072 | 1 | ttgtccaggctggagtgaag | tgg |
| 25288083 | 1 | ggagtgaagtggcatgttca | tgg |
| 25288104 | −1 | acctgaacccagtgaggttg | agg |
| 25288107 | 1 | tcactcagcctcaacctcac | tgg |
| 25288108 | 1 | cactcagcctcaacctcact | ggg |
| 25288110 | −1 | aggatcacctgaacccagtg | agg |
| 25288114 | 1 | gcctcaacctcactgggttc | agg |
| 25288130 | −1 | acttgggaggctgaggcagg | agg |
| 25288133 | −1 | gctacttgggaggctgaggc | agg |
| 25288137 | −1 | cccagctacttgggaggctg | agg |
| 25288143 | −1 | cacagtcccagctacttggg | agg |
| 25288146 | −1 | acccacagtcccagctactt | ggg |
| 25288147 | 1 | gcctcagcctoccaagtagc | tgg |
| 25288147 | −1 | cacccacagtcccagctact | tgg |
| 25288148 | 1 | cctcagcctcccaagtagct | ggg |
| 25288155 | 1 | ctcccaagtagctgggactg | tgg |
| 25288156 | 1 | tcccaagtagctgggactgt | ggg |
| 25288174 | −1 | gaaaaatcagctaggcgtgg | tgg |
| 25288177 | −1 | atagaaaaatcagctaggcg | tgg |
| 25288182 | −1 | agaaaatagaaaaatcagct | agg |
| 25288204 | 1 | tttctattttctgcagagac | agg |
| 25288217 | −1 | ccagcctgagcaacacagtg | agg |
| 25288224 | 1 | aggacctcactgtgttgctc | agg |
| 25288228 | 1 | cctcactgtgttgctcaggc | tgg |
| 25288242 | 1 | tcaggctggtctcaaactcc | tgg |
| 25288243 | 1 | caggctggtctcaaactcct | ggg |
| 25288249 | −1 | tgggcagatcacttgagccc | agg |
| 25288266 | 1 | ctcaagtgatctgcccacct | cgg |
| 25288268 | −1 | gtacttttcagagccgaggt | ggg |
| 25288269 | −1 | agtacttttcagagccgagg | tgg |
| 25288272 | −1 | tccagtacttttcagagccg | agg |
| 25288282 | 1 | acctcggctctgaaaagtac | tgg |
| 25288302 | −1 | tgtggtctcagctactcagg | agg |
| 25288305 | −1 | gcctgtggtctcagctactc | agg |
| 25288315 | 1 | tcctgagtagctgagaccac | agg |
| 25288320 | −1 | ggtgtggtggtgtgtgcctg | tgg |
| 25288333 | −1 | aaaaaaaaagctaggtgtgg | tgg |
| 25288336 | −1 | aaaaaaaaaaaagctaggtg | tgg |
| 25288341 | −1 | aagcaaaaaaaaaaaaagct | agg |
| 25288365 | 1 | ttttttgctttttgtagaga | tgg |
| 25288386 | 1 | ggagtctcactatgttgccc | agg |
| 25288390 | 1 | tctcactatgttgcccaggc | tgg |
| 25288392 | −1 | ctggagtttgagaccagcct | ggg |
| 25288393 | −1 | cctggagtttgagaccagcc | tgg |
| 25288404 | 1 | ccaggctggtctcaaactcc | agg |
| 25288411 | −1 | tgggaggattgcttaaggcc | tgg |
| 25288416 | −1 | tgaggtgggaggattgctta | agg |
| 25288427 | −1 | ctttgggaggctgaggtggg | agg |
| 25288430 | −1 | gcactttgggaggctgaggt | ggg |
| 25288431 | −1 | cgcactttgggaggctgagg | tgg |
| 25288434 | −1 | cttcgcactttgggaggctg | agg |
| 25288440 | −1 | tgtaatcttcgcactttggg | agg |
| 25288443 | −1 | acctgtaatcttcgcacttt | ggg |
| 25288444 | −1 | cacctgtaatcttcgcactt | tgg |
| 25288453 | 1 | tcccaaagtgcgaagattac | agg |
| 25288471 | −1 | ACTTTTAAggccaggaatgg | tgg |
| 25288472 | 1 | caggtgtgagccaccattcc | tgg |
| 25288474 | −1 | CACACTTTTAAggccaggaa | tgg |
| 25288479 | −1 | AATATCACACTTTTAAggcc | agg |
| 25288484 | −1 | TTAAAAATATCACACTTTTA | Agg |
| 25288515 | 1 | TAATGTATTTTGAAATCTGC | AGG |
| 25288532 | −1 | GTTATTGCTATTATCTTCTA | GGG |
| 25288533 | −1 | GGTTATTGCTATTATCTTCT | AGG |
| 25288554 | −1 | gtcaagcacAATAAAGGAGT | TGG |
| 25288560 | −1 | atatacgtcaagcacAATAA | AGG |
| 25288595 | 1 | aactcactttgcccttaccg | tgg |
| 25288595 | −1 | tgcctctggagccacggtaa | ggg |
| 25288596 | −1 | atgcctctggagccacggta | agg |
| 25288601 | −1 | acccaatgcctctggagcca | cgg |
| 25288604 | 1 | tgcccttaccgtggctccag | agg |
| 25288609 | −1 | taaggtggacccaatgcctc | tgg |
| 25288610 | 1 | taccgtggctccagaggcat | tgg |
| 25288611 | 1 | accgtggctccagaggcatt | ggg |
| 25288624 | −1 | tggtgcctccatttataagg | tgg |
| 25288627 | 1 | cattgggtccaccttataaa | tgg |
| 25288627 | −1 | ccttggtgcctccatttata | agg |
| 25288630 | 1 | tgggtccaccttataaatgg | agg |
| 25288638 | 1 | ccttataaatggaggcacca | agg |
| 25288644 | −1 | tatttaatcactctgtgcct | tgg |
| 25288666 | 1 | agtgattaaataaattgccc | agg |
| 25288672 | −1 | ctttctggctgtgtgatcct | ggg |
| 25288673 | −1 | actttctggctgtgtgatcc | tgg |
| 25288687 | −1 | atcttgactcagacactttc | tgg |
| 25288709 | 1 | ctgagtcaagattccagccc | ags |
| 25288711 | −1 | caggtctaggctgcctgggc | tgg |
| 25288715 | −1 | ctctcaggtctaggctgcct | ggg |
| 25288716 | −1 | gctctcaggtctaggctgcc | tgg |
| 25288724 | −1 | aggagcgtgctctcaggtct | agg |
| 25288730 | −1 | gtggttaggagcgtgctctc | agg |
| 25288744 | −1 | Gacagtgatgtgcagtggtt | agg |
| 25288749 | −1 | GCTAAGacagtgatgtgcag | tgg |
| 25288773 | −1 | AGGGCCAGTTTGTGCTGAGG | AGG |
| 25288776 | −1 | TCAAGGGCCAGTTTGTGCTG | AGG |
| 25288780 | 1 | AGCACCTCCTCAGCACAAAC | TGG |
| 25288789 | 1 | TCAGCACAAACTGGCCCTTG | AGG |
| 25288792 | −1 | GGCGGTATTTCATTCCTCAA | GGG |
| 25288793 | −1 | CGGCGGTATTTCATTCCTCA | AGG |
| 25288808 | 1 | GAGGAATGAAATACCGCCGC | CGG |
| 25288810 | −1 | AGGAGCGTGTGTGCCGGCGG | CGG |
| 25288813 | −1 | CTCAGGAGCGTGTGTGCCGG | CGG |
| 25288816 | −1 | TAACTCAGGAGCGTGTGTGC | CGG |
| 25288830 | −1 | CATTGACAAAGGCTTAACTC | AGG |
| 25288841 | −1 | GGTGTTCATTTCATTGACAA | AGG |
| 25288862 | −1 | ACAGGTTATTCCTTTTAAGT | GGG |
| 25288863 | 1 | GAAATGAACACCCACTTAAA | AGG |
| 25288863 | −1 | GACAGGTTATTCCTTTTAAG | TGG |
| 25288878 | 1 | TTAAAAGGAATAACCTGTCC | AGG |
| 25288880 | −1 | ATGTTCCATCGTGCCTGGAC | AGG |
| 25288885 | −1 | ACTCAATGTTCCATCGTGCC | TGG |
| 25288886 | 1 | AATAACCTGTCCAGGCACGA | TGG |
| 25288910 | −1 | GACCAGGAATTTAGAATAAG | GGG |
| 25288911 | −1 | GGACCAGGAATTTAGAATAA | GGG |
| 25288912 | −1 | GGGACCAGGAATTTAGAATA | AGG |
| 25288919 | 1 | AACCCCTTATTCTAAATTCC | TGG |
| 25288926 | −1 | GAAGGAGTCTTACAGGGACC | AGG |
| 25288932 | −1 | CATGGGGAAGGAGTCTTACA | GGG |
| 25288933 | −1 | GCATGGGGAAGGAGTCTTAC | AGG |
| 25288944 | −1 | AAAGGGCAAGGGCATGGGGA | AGG |
| 25288948 | −1 | CAGAAAAGGGCAAGGGCATG | GGG |
| 25288949 | −1 | TCAGAAAAGGGCAAGGGCAT | GGG |
| 25288950 | −1 | GTCAGAAAAGGGCAAGGGCA | TGG |
| 25288955 | −1 | GGAAGGTCAGAAAAGGGCAA | GGG |
| 25288956 | −1 | GGGAAGGTCAGAAAAGGGCA | AGG |
| 25288961 | −1 | TTTAGGGGAAGGTCAGAAAA | GGG |
| 25288962 | −1 | CTTTAGGGGAAGGTCAGAAA | AGG |
| 25288972 | −1 | GCCTCAAGGACTTTAGGGGA | AGG |
| 25288976 | −1 | TTAAGCCTCAAGGACTTTAG | GGG |
| 25288977 | −1 | CTTAAGCCTCAAGGACTTTA | GGG |
| 25288978 | −1 | GCTTAAGCCTCAAGGACTTT | AGG |
| 25288982 | 1 | ACCTTCCCCTAAAGTCCTTG | AGG |
| 25288986 | −1 | CTATGCCCGCTTAAGCCTCA | AGG |
| 25288991 | 1 | TAAAGTCCTTGAGGCTTAAG | CGG |
| 25288992 | 1 | AAAGTCCTTGAGGCTTAAGC | GGG |
| 25289014 | 1 | GCATAGTCTGCAGCAAACAC | TGG |
| 25289015 | 1 | CATAGTCTGCAGCAAACACT | GGG |
| 25289016 | 1 | ATAGTCTGCAGCAAACACTG | GGG |
| 25289037 | −1 | aaagcctgtgctctgaaGTC | TGG |
| 25289044 | 1 | GAGTCCAGACttcagagcac | agg |
| 25289050 | 1 | AGACttcagagcacaggctt | tgg |
| 25289057 | 1 | agagcacaggctttggatct | agg |
| 25289065 | 1 | ggctttggatctaggccagc | tgg |
| 25289069 | −1 | atgtgaggttcaaatccagc | tgg |
| 25289084 | −1 | gccagctgatcacaaatgtg | agg |
| 25289094 | 1 | acctcacatttgtgatcagc | tgg |
| 25289117 | −1 | gaggattaaaatggactttt | tgg |
| 25289126 | −1 | ggtcacgtagaggattaaaa | tgg |
| 25289136 | −1 | ttttacagagggtcacgtag | agg |
| 25289147 | −1 | tcagtatcccattttacaga | ggg |
| 25289148 | −1 | ttcagtatcccattttacag | agg |
| 25289150 | 1 | ctacgtgaccctctgtaaaa | tgg |
| 25289151 | 1 | tacgtgaccctctgtaaaat | ggg |
| 25289162 | 1 | ctgtaaaatgggatactgaa | tgg |
| 25289213 | 1 | attttttttgtgtgtgtgtg | agg |
| 25289235 | 1 | gcagtcttactctgttgccc | agg |
| 25289239 | 1 | tcttactctgttgcccaggc | tgg |
| 25289241 | −1 | gcaccactgcactccagcct | ggg |
| 25289242 | −1 | tgcaccactgcactccagcc | tgg |
| 25289249 | 1 | ttgcccaggctggagtgcag | tgg |
| 25289260 | 1 | ggagtgcagtggtgcagtct | cgg |
| 25289272 | −1 | cgggaggcagaggtttcagt | ggg |
| 25289273 | −1 | ccgggaggcagaggtttcag | tgg |
| 25289282 | −1 | cgcttgaacccgggaggcag | agg |
| 25289284 | 1 | ccactgaaacctctgcctcc | cgg |
| 25289285 | 1 | cactgaaacctctgcctccc | ggg |
| 25289288 | −1 | ggcagtcgcttgaacccggg | agg |
| 25289291 | −1 | catggcagtcgcttgaaccc | ggg |
| 25289292 | −1 | gcatggcagtcgcttgaacc | cgg |
| 25289309 | −1 | ccactctcgaggctgaggca | tgg |
| 25289314 | −1 | cccagccactctcgaggctg | agg |
| 25289320 | 1 | ccatgcctcagcctcgagag | tgg |
| 25289320 | −1 | tgtaatcccagccactctcg | agg |
| 25289324 | 1 | gcctcagcctcgagagtggc | tgg |
| 25289325 | 1 | cctcagcctcgagagtggct | ggg |
| 25289351 | −1 | caaaaattacccgggcatgg | tgg |
| 25289352 | 1 | caagcatgcaccaccatgcc | cgg |
| 25289353 | 1 | aagcatgcaccaccatgccc | ggg |
| 25289354 | −1 | atacaaaaattacccgggca | tgg |
| 25289359 | −1 | taaaaatacaaaaattaccc | ggg |
| 25289360 | −1 | ctaaaaatacaaaaattacc | cgg |
| 25289396 | 1 | gagacagagtttcaccatgt | tgg |
| 25289399 | −1 | caagagtggcctggccaaca | tgg |
| 25289401 | 1 | agagtttcaccatgttggcc | agg |
| 25289408 | −1 | ccaggggttcaagagtggcc | tgg |
| 25289413 | −1 | tgaggccaggggttcaagag | tgg |
| 25289419 | 1 | ccaggccactcttgaacccc | tgg |
| 25289424 | −1 | ggtggatcacttgaggccag | ggg |
| 25289425 | −1 | aggtggatcacttgaggcca | ggg |
| 25289426 | −1 | caggtggatcacttgaggcc | agg |
| 25289431 | −1 | caaggcaggtggatcacttg | agg |
| 25289442 | −1 | ctttgggaggccaaggcagg | tgg |
| 25289443 | 1 | ctcaagtgatccacctgcct | tgg |
| 25289445 | −1 | gcactttgggaggccaaggc | agg |
| 25289449 | −1 | cccagcactttgggaggcca | agg |
| 25289455 | −1 | tgtactcccagcactttggg | agg |
| 25289458 | −1 | gcctgtactcccagcacttt | ggg |
| 25289459 | 1 | gccttggcctcccaaagtgc | tgg |
| 25289459 | −1 | tgcctgtactcccagcactt | tgg |
| 25289460 | 1 | ccttggcctcccaaagtgct | ggg |
| 25289468 | 1 | tcccaaagtgctgggagtac | agg |
| 25289486 | −1 | ccctataaggctgggtgcag | tgg |
| 25289494 | −1 | aattttaaccctataaggct | ggg |
| 25289495 | −1 | aaattttaaccctataaggc | tgg |
| 25289496 | 1 | gccactgcacccagccttat | agg |
| 25289497 | 1 | ccactgcacccagccttata | ggg |
| 25289499 | −1 | ttttaaattttaaccctata | agg |
| 25289514 | 1 | atagggttaaaatttaaaag | agg |
| 25289541 | −1 | ataagagcattttgtaaaac | agg |
| 25289582 | 1 | CATTATCATCACTGTTGCTG | TGG |
| 25289613 | 1 | TCATCATCATTAACTCCCAG | AGG |
| 25289614 | 1 | CATCATCATTAACTCCCAGA | GGG |
| 25289617 | 1 | CATCATTAACTCCCAGAGGG | AGG |
| 25289617 | −1 | TGAGACTCCCTCCTCCCTCT | GGG |
| 25289618 | −1 | CTGAGACTCCCTCCTCCCTC | TGG |
| 25289620 | 1 | CATTAACTCCCAGAGGGAGG | AGG |
| 25289621 | 1 | ATTAACTCCCAGAGGGAGGA | GGG |
| 25289644 | 1 | AGTCTCAGAGCAAGCTGCTC | AGG |
| 25289645 | 1 | GTCTCAGAGCAAGCTGCTCA | GGG |
| 25289646 | 1 | TCTCAGAGCAAGCTGCTCAG | GGG |
| 25289653 | 1 | GCAAGCTGCTCAGGGGAGAC | TGG |
| 25289663 | 1 | CAGGGGAGACTGGATGTCCA | TGG |
| 25289669 | −1 | gtactgagctgGACAATCCA | TGG |
| 25289680 | −1 | tggaggaagtggtactgagc | tgG |
| 25289691 | −1 | ggaggacttcctggaggaag | tgg |
| 25289693 | 1 | agctcagtaccacttcctcc | agg |
| 25289697 | −1 | tatcagggaggacttcctgg | agg |
| 25289700 | −1 | acttatcagggaggacttcc | tgg |
| 25289709 | −1 | gctgactggacttatcaggg | agg |
| 25289712 | −1 | gatgctgactggacttatca | ggg |
| 25289713 | −1 | tgatgctgactggacttatc | agg |
| 25289723 | −1 | aaggagagggtgatgctgac | tgg |
| 25289736 | −1 | tggggttcattggaaggaga | ggg |
| 25289737 | −1 | gtggggttcattggaaggag | agg |
| 25289742 | −1 | ggctagtggggttcattgga | agg |
| 25289746 | −1 | ACaaggctagtggggttcat | tgg |
| 25289754 | −1 | GTGATATCACaaggctagtg | ggg |
| 25289755 | −1 | TGTGATATCACaaggctagt | ggg |
| 25289756 | −1 | CTGTGATATCACaaggctag | tgg |
| 25289763 | −1 | AGAATATCTGTGATATCACa | agg |
| 25289785 | 1 | CACAGATATTCTTAGTTGAC | AGG |
| 25289792 | 1 | ATTCTTAGTTGACAGGCTCA | TGG |
| 25289809 | −1 | aaTGTACTTATGATCTAGAC | AGG |
| 25289834 | 1 | AAGTACAttttttttttttt | tGG |
| 25289865 | −1 | TCAGGAGTAGAAAATTATTT | TGG |
| 25289883 | −1 | TTTGACCAATGAGCATGCTC | AGG |
| 25289889 | 1 | CTACTCCTGAGCATGCTCAT | TGG |
| 25289896 | 1 | TGAGCATGCTCATTGGTCAA | AGG |
| 25289900 | 1 | CATGCTCATTGGTCAAAGGA | AGG |
| 25289904 | 1 | CTCATTGGTCAAAGGAAGGA | AGG |
| 25289926 | 1 | GAATCATAATAGCGTtaata | agg |
| 25289948 | 1 | gctagcgtcttttcagaagt | tgg |
| 25289967 | 1 | ttggttctttgtgccagtct | tgg |
| 25289969 | −1 | ggtgtgtctagcaccaagac | tgg |
| 25289986 | 1 | ttggtgctagacacaccgat | agg |
| 25289990 | −1 | tgaaggagtattcttcctat | cgg |
| 25290007 | −1 | ttggtgtcctggggatgtga | agg |
| 25290011 | 1 | gaatactccttcacatcccc | agg |
| 25290016 | −1 | tatcccatgttggtgtcctg | ggg |
| 25290017 | −1 | gtatcccatgttggtgtcct | ggg |
| 25290018 | −1 | cgtatcccatgttggtgtcc | tgg |
| 25290023 | 1 | acatccccaggacaccaaca | tgg |
| 25290024 | 1 | catccccaggacaccaacat | ggg |
| 25290026 | −1 | tgatcaaacgtatcccatgt | tgg |
| 25290058 | 1 | catcattcttaatttgcaga | agg |
| 25290067 | 1 | taatttgcagaaggagaaat | agg |
| 25290097 | 1 | agatgaaatagccactccag | tgg |
| 25290097 | −1 | tcccagccttgccactggag | tgg |
| 25290102 | 1 | aaatagccactccagtggca | agg |
| 25290102 | −1 | tccagtcccagccttgccac | tgg |
| 25290106 | 1 | agccactccagtggcaaggc | tgg |
| 25290107 | 1 | gccactccagtggcaaggct | ggg |
| 25290112 | 1 | tccagtggcaaggctgggac | tgg |
| 25290119 | 1 | gcaaggctgggactggaagc | cgg |
| 25290120 | 1 | caaggctgggactggaagcc | ggg |
| 25290127 | −1 | atttggaatcaggacaagcc | cgg |
| 25290137 | −1 | aagaaactggatttggaatc | agg |
| 25290144 | −1 | cagtggaaagaaactggatt | tgg |
| 25290150 | −1 | Ccgtggcagtggaaagaaac | tgg |
| 25290161 | 1 | ccagtttctttccactgcca | cgG |
| 25290161 | −1 | TCTCTCCGTCTCcgtggcag | tgg |
| 25290167 | 1 | tctttccactgccacgGAGA | CGG |
| 25290167 | −1 | GTCCCTTCTCTCCGTCTCcg | tgg |
| 25290175 | 1 | ctgccacgGAGACGGAGAGA | AGG |
| 25290176 | 1 | tgccacgGAGACGGAGAGAA | GGG |
| 25290183 | 1 | GAGACGGAGAGAAGGGACAG | TGG |
| 25290193 | 1 | GAAGGGACAGTGGCCCCAGA | TGG |
| 25290194 | 1 | AAGGGACAGTGGCCCCAGAT | GGG |
| 25290195 | 1 | AGGGACAGTGGCCCCAGATG | GGG |
| 25290195 | −1 | AGTCACCCCATCCCCATCTG | GGG |
| 25290196 | −1 | CAGTCACCCCATCCCCATCT | GGG |
| 25290197 | −1 | CCAGTCACCCCATCCCCATC | TGG |
| 25290199 | 1 | ACAGTGGCCCCAGATGGGGA | TGG |
| 25290200 | 1 | CAGTGGCCCCAGATGGGGAT | GGG |
| 25290201 | 1 | AGTGGCCCCAGATGGGGATG | GGG |
| 25290208 | 1 | CCAGATGGGGATGGGGTGAC | TGG |
| 25290214 | 1 | GGGGATGGGGTGACTGGATG | TGG |
| 25290215 | 1 | GGGATGGGGTGACTGGATGT | GGG |
| 25290219 | 1 | TGGGGTGACTGGATGTGGGC | AGG |
| 25290226 | 1 | ACTGGATGTGGGCAGGCCTG | CGG |
| 25290227 | 1 | CTGGATGTGGGCAGGCCTGC | GGG |
| 25290228 | 1 | TGGATGTGGGCAGGCCTGCG | GGG |
| 25290229 | 1 | GGATGTGGGCAGGCCTGCGG | GGG |
| 25290231 | −1 | AGAGGGCACTCTTCCCCCGC | AGG |
| 25290248 | −1 | TCATTCGGATGCTCAACAGA | GGG |
| 25290249 | −1 | ATCATTCGGATGCTCAACAG | AGG |
| 25290262 | 1 | TCTGTTGAGCATCCGAATGA | TGG |
| 25290263 | −1 | TCTTTTCTGCTGCCATCATT | CGG |
| 25290281 | 1 | ATGGCAGCAGAAAAGAAGAC | TGG |
| 25290282 | 1 | TGGCAGCAGAAAAGAAGACT | GGG |
| 25290300 | −1 | CCTCAGGGGATCTGATAACT | GGG |
| 25290301 | −1 | CCCTCAGGGGATCTGATAAC | TGG |
| 25290311 | 1 | CCCAGTTATCAGATCCCCTG | AGG |
| 25290312 | 1 | CCAGTTATCAGATCCCCTGA | GGG |
| 25290314 | −1 | CGGGGTGACTGTTCCCTCAG | GGG |
| 25290315 | −1 | TCGGGGTGACTGTTCCCTCA | GGG |
| 25290316 | −1 | ATCGGGGTGACTGTTCCCTC | AGG |
| 25290332 | −1 | CATCTGACTGAGGGTGATCG | GGG |
| 25290333 | −1 | TCATCTGACTGAGGGTGATC | GGG |
| 25290334 | −1 | CTCATCTGACTGAGGGTGAT | CGG |
| 25290341 | −1 | ACACACACTCATCTGACTGA | GGG |
| 25290342 | −1 | TACACACACTCATCTGACTG | AGG |
| 25290373 | −1 | cctcagtgccttcatctatg | aGG |
| 25290376 | 1 | GATCAATGCCtcatagatga | agg |
| 25290384 | 1 | CCtcatagatgaaggcactg | agg |
| 25290394 | 1 | gaaggcactgaggcacagag | tgg |
| 25290418 | −1 | gcaccctgagccatgtggtc | tgg |
| 25290419 | 1 | aagtcatctgccagaccaca | tgg |
| 25290423 | −1 | Cctctgcaccctgagccatg | tgg |
| 25290425 | 1 | tctgccagaccacatggctc | agg |
| 25290426 | 1 | ctgccagaccacatggctca | ggg |
| 25290434 | 1 | ccacatggctcagggtgcag | agG |
| 25290446 | 1 | gggtgcagagGCCACCTTAA | CGG |
| 25290446 | −1 | CATCTCTTCTCCCGTTAAGG | TGG |
| 25290447 | 1 | ggtgcagagGCCACCTTAAC | GGG |
| 25290449 | −1 | GACCATCTCTTCTCCCGTTA | AGG |
| 25290458 | 1 | CACCTTAACGGGAGAAGAGA | TGG |
| 25290475 | −1 | TGGGCGCTGATGCTGCAGAG | TGG |
| 25290488 | 1 | ACTCTGCAGCATCAGCGCCC | AGG |
| 25290491 | 1 | CTGCAGCATCAGCGCCCAGG | Tgg |
| 25290492 | 1 | TGCAGCATCAGCGCCCAGGT | ggg |
| 25290494 | −1 | gacaagatttctacccACCT | GGG |
| 25290495 | −1 | agacaagatttctacccACC | TGG |
| 25290524 | −1 | gttgggcacctactttctgt | ggg |
| 25290525 | −1 | tgttgggcacctactttctg | tgg |
| 25290527 | 1 | cttctattcccacagaaagt | agg |
| 25290541 | −1 | ttctttcaacaaacactgtt | ggg |
| 25290542 | −1 | attctttcaacaaacactgt | tgg |
| 25290591 | 1 | tgaatgaatgaatgagtgaG | AGG |
| 25290606 | −1 | GCCAGGACGACTGAGAAGGA | AGG |
| 25290610 | −1 | GAGAGCCAGGACGACTGAGA | AGG |
| 25290616 | 1 | TCCTTCCTTCTCAGTCGTCC | TGG |
| 25290623 | −1 | TGGGGGAGAGAGGGAGAGCC | AGG |
| 25290632 | −1 | GCCGAATACTGGGGGAGAGA | GGG |
| 25290633 | −1 | AGCCGAATACTGGGGGAGAG | AGG |
| 25290640 | −1 | GGTGGCCAGCCGAATACTGG | GGG |
| 25290641 | −1 | TGGTGGCCAGCCGAATACTG | GGG |
| 25290642 | 1 | TCCCTCTCTCCCCCAGTATT | CGG |
| 25290642 | −1 | ATGGTGGCCAGCCGAATACT | GGG |
| 25290643 | −1 | CATGGTGGCCAGCCGAATAC | TGG |
| 25290646 | 1 | TCTCTCCCCCAGTATTCGGC | TGG |
| 25290658 | −1 | CACCGACAAAGCACTCATGG | TGG |
| 25290661 | −1 | CAGCACCGACAAAGCACTCA | TGG |
| 25290667 | 1 | GGCCACCATGAGTGCTTTGT | CGG |
| 25290682 | 1 | TTTGTCGGTGCTGATCTCAG | TGG |
| 25290694 | 1 | GATCTCAGTGGATGCTGTCT | TGG |
| 25290695 | 1 | ATCTCAGTGGATGCTGTCTT | GGG |
| 25290696 | 1 | TCTCAGTGGATGCTGTCTTG | GGG |
| 25290700 | 1 | AGTGGATGCTGTCTTGGGGA | AGG |
| 25290709 | 1 | TGTCTTGGGGAAGGTCAACT | TGG |
| 25290718 | 1 | GAAGGTCAACTTGGCGCAGT | TGG |
| 25290721 | 1 | GGTCAACTTGGCGCAGTTGG | TGG |
| 25290727 | 1 | CTTGGCGCAGTTGGTGGTGA | TGG |
| 25290733 | 1 | GCAGTTGGTGGTGATGGTGC | TGG |
| 25290736 | 1 | GTTGGTGGTGATGGTGCTGG | TGG |
| 25290739 | 1 | GGTGGTGATGGTGCTGGTGG | AGG |
| 25290752 | 1 | CTGGTGGAGGTGACAGCTTT | AGG |
| 25290762 | 1 | TGACAGCTTTAGGCAACCTG | AGG |
| 25290766 | 1 | AGCTTTAGGCAACCTGAGGA | TGG |
| 25290767 | −1 | TATTACTGATGACCATCCTC | AGG |
| 25290797 | 1 | AATATCTTCAACGTGAGTCA | TGG |
| 25290803 | 1 | TTCAACGTGAGTCATGGTGC | TGG |
| 25290804 | 1 | TCAACGTGAGTCATGGTGCT | GGG |
| 25290807 | 1 | ACGTGAGTCATGGTGCTGGG | AGG |
| 25290810 | 1 | TGAGTCATGGTGCTGGGAGG | AGG |
| 25290811 | 1 | GAGTCATGGTGCTGGGAGGA | GGG |
| 25290817 | 1 | TGGTGCTGGGAGGAGGGACC | TGG |
| 25290818 | 1 | GGTGCTGGGAGGAGGGACCT | GGG |
| 25290824 | −1 | GCTTTTGGCCCTTTTCTCCC | AGG |
| 25290826 | 1 | GAGGAGGGACCTGGGAGAAA | AGG |
| 25290827 | 1 | AGGAGGGACCTGGGAGAAAA | GGG |
| 25290839 | −1 | ACCCCACCAAATGGAGCTTT | TGG |
| 25290844 | 1 | AAAGGGCCAAAAGCTCCATT | TGG |
| 25290847 | 1 | GGGCCAAAAGCTCCATTTGG | TGG |
| 25290848 | 1 | GGCCAAAAGCTCCATTTGGT | GGG |
| 25290848 | −1 | ACCCTGGAAACCCCACCAAA | TGG |
| 25290849 | 1 | GCCAAAAGCTCCATTTGGTG | GGG |
| 25290857 | 1 | CTCCATTTGGTGGGGTTTCC | AGG |
| 25290858 | 1 | TCCATTTGGTGGGGTTTCCA | GGG |
| 25290864 | −1 | GTCTTTATTTTTCAAAACCC | TGG |
| 25290889 | −1 | tcccaagtagctgggattac | agg |
| 25290897 | −1 | cctcaacctcccaagtagct | ggg |
| 25290898 | 1 | AAcctgtaatcccagctact | tgg |
| 25290898 | −1 | tcctcaacctcccaagtagc | tgg |
| 25290899 | 1 | Acctgtaatcccagctactt | ggg |
| 25290902 | 1 | tgtaatcccagctacttggg | agg |
| 25290908 | 1 | cccagctacttgggaggttg | agg |
| 25290911 | 1 | agctacttgggaggttgagg | agg |
| 25290912 | 1 | gctacttgggaggttgagga | ggg |
| 25290926 | 1 | tgaggagggaagatcacttg | agg |
| 25290931 | 1 | agggaagatcacttgaggcc | agg |
| 25290938 | −1 | ccaggctggtctcaaactcc | tgg |
| 25290949 | 1 | ccaggagtttgagaccagcc | tgg |
| 25290950 | 1 | caggagtttgagaccagcct | ggg |
| 25290952 | −1 | tcttgctatgatgcccaggc | tgg |
| 25290956 | −1 | aggatcttgctatgatgccc | agg |
| 25290976 | −1 | aaaattactttttagagatg | agg |
| 25291008 | 1 | ttttctaaattatccagttg | tgg |
| 25291010 | −1 | caggtgcatgccaccacaac | tgg |
| 25291011 | 1 | tctaaattatccagttgtgg | tgg |
| 25291029 | −1 | tcctgagtaactgagactac | agg |
| 25291039 | 1 | acctgtagtctcagttactc | agg |
| 25291042 | 1 | tgtagtctcagttactcagg | agg |
| 25291048 | 1 | ctcagttactcaggaggctg | agg |
| 25291058 | 1 | caggaggctgaggtgtgagt | tgg |
| 25291062 | 1 | aggctgaggtgtgagttgga | agg |
| 25291078 | 1 | tggaaggattgtttgagccc | agg |
| 25291084 | −1 | cagctcggtccctaactcct | ggg |
| 25291085 | 1 | attgtttgagcccaggagtt | agg |
| 25291085 | −1 | ccagctcggtccctaactcc | tgg |
| 25291086 | 1 | ttgtttgagcccaggagtta | ggg |
| 25291096 | 1 | ccaggagttagggaccgagc | tgg |
| 25291097 | 1 | caggagttagggaccgagct | ggg |
| 25291099 | −1 | tcttgctatgttgcccagct | cgg |
| 25291122 | −1 | tacctatttatttagagatg | agg |
| 25291131 | 1 | gacctcatctctaaataaat | agg |
| 25291135 | 1 | tcatctctaaataaataggt | agg |
| 25291138 | 1 | tctctaaataaataggtagg | tgg |
| 25291183 | 1 | agacagacagacagacagac | agg |
| 25291187 | 1 | agacagacagacagacaggc | tgg |
| 25291188 | 1 | gacagacagacagacaggct | ggg |
| 25291196 | 1 | gacagacaggctgggtacag | tgg |
| 25291214 | −1 | tcccaaagtgctgggattac | agg |
| 25291222 | −1 | ccttggcctcccaaagtgct | ggg |
| 25291223 | 1 | cacctgtaatcccagcactt | tgg |
| 25291223 | −1 | tccttggcctcccaaagtgc | tgg |
| 25291224 | 1 | acctgtaatcccagcacttt | ggg |
| 25291227 | 1 | tgtaatcccagcactttggg | agg |
| 25291233 | 1 | cccagcactttgggaggcca | agg |
| 25291236 | 1 | agcactttgggaggccaagg | agg |
| 25291237 | 1 | gcactttgggaggccaagga | ggg |
| 25291239 | −1 | ctcaggtgatctgccctcct | tgg |
| 25291251 | 1 | caaggagggcagatcacctg | agg |
| 25291256 | 1 | agggcagatcacctgaggtc | agg |
| 25291256 | −1 | ggtcttgaactcctgacctc | agg |
| 25291274 | 1 | tcaggagttcaagaccagcc | tgg |
| 25291277 | −1 | ttcccccatgttgaccaggc | tgg |
| 25291281 | −1 | gaggttcccccatgttgacc | agg |
| 25291283 | 1 | caagaccagcctggtcaaca | tgg |
| 25291284 | 1 | aagaccagcctggtcaacat | ggg |
| 25291285 | 1 | agaccagcctggtcaacatg | ggg |
| 25291286 | 1 | gaccagcctggtcaacatgg | ggg |
| 25291300 | −1 | ttgtatttttagtagagatg | agg |
| 25291322 | 1 | ctaaaaatacaaaatttagc | tgg |
| 25291323 | 1 | taaaaatacaaaatttagct | ggg |
| 25291328 | 1 | atacaaaatttagctgggca | tgg |
| 25291331 | 1 | caaaatttagctgggcatgg | tgg |
| 25291335 | 1 | atttagctgggcatggtggc | agg |
| 25291349 | −1 | tcctgagtagctgggattac | agg |
| 25291357 | −1 | cctcagcctcctgagtagct | ggg |
| 25291358 | −1 | gcctcagcctcctgagtagc | tgg |
| 25291359 | 1 | gcctgtaatcccagctactc | agg |
| 25291362 | 1 | tgtaatcccagctactcagg | agg |
| 25291368 | 1 | cccagctactcaggaggctg | agg |
| 25291394 | 1 | gagaatcgcttgaacccgag | agg |
| 25291397 | 1 | aatcgcttgaacccgagagg | tgg |
| 25291397 | −1 | cactgcaacctccacctctc | ggg |
| 25291398 | −1 | tcactgcaacctccacctct | cgg |
| 25291400 | 1 | cgcttgaacccgagaggtgg | agg |
| 25291422 | −1 | gcagtgcaatggcgcgatct | cgg |
| 25291433 | −1 | tcccccaggctgcagtgcaa | tgg |
| 25291440 | 1 | cgcgccattgcactgcagcc | tgg |
| 25291441 | 1 | gcgccattgcactgcagcct | ggg |
| 25291442 | 1 | cgccattgcactgcagcctg | ggg |
| 25291443 | 1 | gccattgcactgcagcctgg | ggg |
| 25291447 | −1 | aagtcttgctcttgtccccc | agg |
| 25291528 | −1 | cacatttttgtaaactcatt | tgg |
| 25291540 | 1 | caaatgagtttacaaaaatg | tgg |
| 25291579 | −1 | actgtagtagttaaaggcat | tgg |
| 25291585 | −1 | gattatactgtagtagttaa | agg |
| 25291603 | 1 | ctactacagtataatcctgt | agg |
| 25291607 | −1 | catgaatagcacaatcctac | agg |
| 25291630 | 1 | tgctattcatgatataatta | tgg |
| 25291669 | −1 | tgctggacccactgctggtg | agg |
| 25291672 | 1 | tctcagagcctcaccagcag | tgg |
| 25291673 | 1 | ctcagagcctcaccagcagt | ggg |
| 25291674 | −1 | aaacttgctggacccactgc | tgg |
| 25291686 | −1 | tgctggctgtacaaacttgc | tgg |
| 25291703 | −1 | cactgactgaaagaagatgc | tgg |
| 25291746 | 1 | aactgcatatgtcctctcat | tgg |
| 25291747 | 1 | actgcatatgtcctctcatt | ggg |
| 25291747 | −1 | cgacaggctctcccaatgag | agg |
| 25291763 | −1 | ttcaaatttagactttcgac | agg |
| 25291776 | 1 | tgtcgaaagtctaaatttga | agg |
| 25291788 | 1 | aaatttgaaggcagctgtga | agg |
| 25291793 | 1 | tgaaggcagctgtgaaggta | agg |
| 25291805 | −1 | tctgggagagccatttggat | tgg |
| 25291806 | 1 | gaaggtaaggccaatccaaa | tgg |
| 25291810 | −1 | gaggatctgggagagccatt | tgg |
| 25291822 | −1 | AGGGTTACAGcagaggatct | ggg |
| 25291823 | −1 | CAGGGTTACAGcagaggatc | tgg |
| 25291829 | −1 | caggGTCAGGGTTACAGcag | agg |
| 25291841 | −1 | tatgtcctcactcaggGTCA | GGG |
| 25291842 | −1 | ctatgtcctcactcaggGTC | AGG |
| 25291847 | 1 | TGTAACCCTGACcctgagtg | agg |
| 25291847 | −1 | gttggctatgtcctcactca | ggG |
| 25291848 | −1 | ggttggctatgtcctcactc | agg |
| 25291865 | −1 | cacctatgagatgggaaggt | tgg |
| 25291869 | −1 | ttctcacctatgagatggga | agg |
| 25291873 | −1 | agctttctcacctatgagat | ggg |
| 25291874 | 1 | agccaaccttcccatctcat | agg |
| 25291874 | −1 | cagctttctcacctatgaga | tgg |
| 25291893 | 1 | taggtgagaaagctgatgcc | tgg |
| 25291898 | 1 | gagaaagctgatgcctggag | agg |
| 25291899 | 1 | agaaagctgatgcctggaga | ggg |
| 25291900 | 1 | gaaagctgatgcctggagag | ggg |
| 25291900 | −1 | ggcagtcccttcccctctcc | agg |
| 25291904 | 1 | gctgatgcctggagagggga | agg |
| 25291905 | 1 | ctgatgcctggagaggggaa | ggg |
| 25291921 | −1 | ctatcttgctatgtgatctt | ggg |
| 25291922 | −1 | actatcttgctatgtgatct | tgg |
| 25291935 | 1 | aagatcacatagcaagatag | tgg |
| 25291952 | −1 | gGAACTGtgggttctcgctt | ggg |
| 25291953 | −1 | tgGAACTGtgggttctcgct | tgg |
| 25291964 | −1 | ctaagccaggctgGAACTGt | ggg |
| 25291965 | −1 | tctaagccaggctgGAACTG | tgg |
| 25291970 | 1 | gagaacccaCAGTTCcagcc | tgg |
| 25291973 | −1 | cactttcttctaagccaggc | tgG |
| 25291977 | −1 | agtgcactttcttctaagcc | agg |
| 25291990 | 1 | tggcttagaagaaagtgcac | tgg |
| 25291996 | 1 | agaagaaagtgcactggact | tgg |
| 25292005 | 1 | tgcactggacttggagtcaa | agg |
| 25292009 | 1 | ctggacttggagtcaaaggc | tgg |
| 25292010 | 1 | tggacttggagtcaaaggct | ggg |
| 25292011 | 1 | ggacttggagtcaaaggctg | ggg |
| 25292030 | −1 | cagggatttatggcagagct | ggg |
| 25292031 | −1 | acagggatttatggcagagc | tgg |
| 25292040 | −1 | cagagtcacacagggattta | tgg |
| 25292048 | −1 | aaattgcccagagtcacaca | ggg |
| 25292049 | −1 | taaattgcccagagtcacac | agg |
| 25292052 | 1 | cataaatccctgtgtgactc | tgg |
| 25292053 | 1 | ataaatccctgtgtgactct | ggg |
| 25292073 | −1 | gaagaaactaaagctctaag | agg |
| 25292099 | 1 | gtttcttcatctgtaatatg | agg |
| 25292100 | 1 | tttcttcatctgtaatatga | ggg |
| 25292120 | 1 | gggtagcagtactaccacat | agg |
| 25292121 | 1 | ggtagcagtactaccacata | ggg |
| 25292123 | −1 | tactccctcaaaaccctatg | tgg |
| 25292129 | 1 | tactaccacatagggttttg | agg |
| 25292130 | 1 | actaccacatagggttttga | ggg |
| 25292196 | −1 | GACACTGAGGCACAGTAAAG | GGG |
| 25292197 | −1 | GGACACTGAGGCACAGTAAA | GGG |
| 25292198 | −1 | GGGACACTGAGGCACAGTAA | AGG |
| 25292209 | −1 | caaagtccttTGGGACACTG | AGG |
| 25292214 | 1 | ACTGTGCCTCAGTGTCCCAa | agg |
| 25292218 | −1 | agtaaaatccaaagtccttT | GGG |
| 25292219 | −1 | gagtaaaatccaaagtcctt | TGG |
| 25292221 | 1 | CTCAGTGTCCCAaaggactt | tgg |
| 25292243 | 1 | gattttactctgagaaatac | agg |
| 25292244 | 1 | attttactctgagaaataca | ggg |
| 25292253 | 1 | tgagaaatacagggagaact | agg |
| 25292254 | 1 | gagaaatacagggagaacta | ggg |
| 25292262 | 1 | cagggagaactagggagtgt | tgg |
| 25292263 | 1 | agggagaactagggagtgtt | ggg |
| 25292269 | 1 | aactagggagtgttgggcag | agg |
| 25292285 | −1 | ttaaaacataagtcagatca | tgg |
| 25292306 | 1 | acttatgttttaagatactc | tgg |
| 25292313 | 1 | ttttaagatactctggcttc | tgg |
| 25292314 | 1 | tttaagatactctggcttct | ggg |
| 25292332 | 1 | ctgggttcagaaaagactga | agg |
| 25292333 | 1 | tgggttcagaaaagactgaa | ggg |
| 25292334 | 1 | gggttcagaaaagactgaag | ggg |
| 25292343 | 1 | aaagactgaaggggcaagag | agg |
| 25292350 | 1 | gaaggggcaagagaggaagc | agg |
| 25292353 | 1 | ggggcaagagaggaagcagg | tgg |
| 25292365 | 1 | gaagcaggtggagaccagag | cgg |
| 25292368 | −1 | gatggcaatcactgccgctc | tgg |
| 25292419 | 1 | gacaatagctgtgagagtga | tgg |
| 25292420 | 1 | acaatagctgtgagagtgat | ggg |
| 25292426 | 1 | gctgtgagagtgatgggaag | tgg |
| 25292430 | 1 | tgagagtgatgggaagtggt | tgg |
| 25292444 | −1 | tctgctattaaaatacagtc | agg |
| 25292464 | 1 | attttaatagcagaattgac | agg |
| 25292487 | 1 | atttgctgatagactgcacg | tgg |
| 25292488 | 1 | tttgctgatagactgcacgt | ggg |
| 25292489 | 1 | ttgctgatagactgcacgtg | ggg |
| 25292492 | 1 | ctgatagactgcacgtgggg | tgg |
| 25292493 | 1 | tgatagactgcacgtggggt | ggg |
| 25292498 | 1 | gactgcacgtggggtgggag | agg |
| 25292499 | 1 | actgcacgtggggtgggaga | ggg |
| 25292516 | 1 | agagggtcaagatgacttca | agg |
| 25292527 | 1 | atgacttcaaggttctcatc | tgg |
| 25292540 | 1 | tctcatctggcacaactcag | cgg |
| 25292547 | 1 | tggcacaactcagcggctgc | tgg |
| 25292561 | −1 | acattccccatctcagtaaa | tgg |
| 25292565 | 1 | gctggtgccatttactgaga | tgg |
| 25292566 | 1 | ctggtgccatttactgagat | ggg |
| 25292567 | 1 | tggtgccatttactgagatg | ggg |
| 25292575 | 1 | tttactgagatggggaatgt | tgg |
| 25292576 | 1 | ttactgagatggggaatgtt | ggg |
| 25292577 | 1 | tactgagatggggaatgttg | ggg |
| 25292580 | 1 | tgagatggggaatgttgggg | tgg |
| 25292581 | 1 | gagatggggaatgttggggt | ggg |
| 25292591 | 1 | atgttggggtgggatagatc | tgg |
| 25292592 | 1 | tgttggggtgggatagatct | ggg |
| 25292595 | 1 | tggggtgggatagatctggg | agg |
| 25292596 | 1 | ggggtgggatagatctggga | ggg |
| 25292612 | −1 | cacattcgacactgaactct | ggg |
| 25292613 | −1 | ccacattcgacactgaactc | tgg |
| 25292624 | 1 | ccagagttcagtgtcgaatg | tgg |
| 25292634 | 1 | gtgtcgaatgtggtagcgtt | agg |
| 25292635 | 1 | tgtcgaatgtggtagcgtta | ggg |
| 25292641 | 1 | atgtggtagcgttagggtta | agg |
| 25292645 | 1 | ggtagcgttagggttaaggt | tgg |
| 25292646 | 1 | gtagcgttagggttaaggtt | ggg |
| 25292647 | 1 | tagcgttagggttaaggttg | ggg |
| 25292648 | 1 | agcgttagggttaaggttgg | ggg |
| 25292651 | 1 | gttagggttaaggttggggg | agg |
| 25292652 | 1 | ttagggttaaggttggggga | ggg |
| 25292653 | 1 | tagggttaaggttgggggag | ggg |
| 25292654 | 1 | agggttaaggttgggggagg | ggg |
| 25292655 | 1 | gggttaaggttgggggaggg | ggg |
| 25292656 | 1 | ggttaaggttgggggagggg | ggg |
| 25292684 | 1 | atgtgtatgaaacatcccag | tgg |
| 25292688 | −1 | ctccattcagtgtctccact | ggg |
| 25292689 | −1 | tctccattcagtgtctccac | tgg |
| 25292697 | 1 | atcccagtggagacactgaa | tgg |
| 25292723 | 1 | tgtacaagtctgaagcttag | tgg |
| 25292728 | 1 | aagtctgaagcttagtggaa | agg |
| 25292733 | 1 | tgaagcttagtggaaaggtt | agg |
| 25292734 | 1 | gaagcttagtggaaaggtta | ggg |
| 25292739 | 1 | ttagtggaaaggttagggct | agg |
| 25292740 | 1 | tagtggaaaggttagggcta | ggg |
| 25292752 | 1 | tagggctagggatataaatt | tgg |
| 25292753 | 1 | agggctagggatataaattt | ggg |
| 25292773 | 1 | gggagttgttacaatacaga | tgg |
| 25292794 | −1 | agtgatctcCTTGGgtctca | tgg |
| 25292797 | 1 | gtttaaagccatgagacCCA | AGg |
| 25292802 | −1 | cactcctgagtgatctcCTT | GGg |
| 25292803 | −1 | tcactcctgagtgatctcCT | TGG |
| 25292809 | 1 | gagacCCAAGgagatcactc | agg |
| 25292816 | 1 | AAGgagatcactcaggagtg | agg |
| 25292830 | 1 | ggagtgaggataaagagaga | tgg |
| 25292831 | 1 | gagtgaggataaagagagat | ggg |
| 25292844 | 1 | gagagatgggaagaagtctg | agg |
| 25292863 | −1 | tctaaaatgcagggtgttct | agg |
| 25292872 | −1 | tgtcccccctctaaaatgca | ggg |
| 25292873 | −1 | atgtcccccctctaaaatgc | agg |
| 25292876 | 1 | tagaacaccctgcattttag | agg |
| 25292877 | 1 | agaacaccctgcattttaga | ggg |
| 25292878 | 1 | gaacaccctgcattttagag | ggg |
| 25292879 | 1 | aacaccctgcattttagagg | ggg |
| 25292880 | 1 | acaccctgcattttagaggg | ggg |
| 25292903 | 1 | acatgtgtaagagccagcaa | agg |
| 25292905 | −1 | cacaattctgtctcctttgc | tgg |
| 25292921 | 1 | aaaggagacagaattgtgct | tgg |
| 25292926 | 1 | agacagaattgtgcttggag | agg |
| 25292930 | 1 | agaattgtgcttggagaggc | agg |
| 25292933 | 1 | attgtgcttggagaggcagg | agg |
| 25292942 | 1 | ggagaggcaggaggaagccc | agg |
| 25292948 | −1 | ccaggacctcacgctctcct | ggg |
| 25292949 | −1 | tccaggacctcacgctctcc | tgg |
| 25292953 | 1 | aggaagcccaggagagcgtg | agg |
| 25292959 | 1 | cccaggagagcgtgaggtcc | tgg |
| 25292963 | 1 | ggagagcgtgaggtcctgga | agg |
| 25292966 | −1 | cctctctttccttgccttcc | agg |
| 25292968 | 1 | gcgtgaggtcctggaaggca | agg |
| 25292977 | 1 | cctggaaggcaaggaaagag | agg |
| 25292978 | 1 | ctggaaggcaaggaaagaga | ggg |
| 25292985 | 1 | gcaaggaaagagagggcccc | agg |
| 25292988 | 1 | aggaaagagagggccccagg | tgg |
| 25292989 | 1 | ggaaagagagggccccaggt | ggg |
| 25292990 | −1 | agcagcattcagcccacctg | ggg |
| 25292991 | −1 | cagcagcattcagcccacct | ggg |
| 25292992 | −1 | tcagcagcattcagcccacc | tgg |
| 25293007 | 1 | gtgggctgaatgctgctgag | agg |
| 25293016 | 1 | atgctgctgagaggtcaagt | cgg |
| 25293022 | 1 | ctgagaggtcaagtcggatg | agg |
| 25293023 | 1 | tgagaggtcaagtcggatga | ggg |
| 25293027 | 1 | aggtcaagtcggatgagggc | tgg |
| 25293028 | 1 | ggtcaagtcggatgagggct | ggg |
| 25293041 | 1 | gagggctgggaagtagccat | tgg |
| 25293046 | −1 | ggtctcctggccaaatccaa | tgg |
| 25293047 | 1 | tgggaagtagccattggatt | tgg |
| 25293052 | 1 | agtagccattggatttggcc | agg |
| 25293059 | −1 | ccatgcatgccaaggtctcc | tgg |
| 25293061 | 1 | tggatttggccaggagacct | tgg |
| 25293067 | −1 | ctctacaaccatgcatgcca | agg |
| 25293070 | 1 | ccaggagaccttggcatgca | tgg |
| 25293079 | 1 | cttggcatgcatggttgtag | agg |
| 25293082 | 1 | ggcatgcatggttgtagagg | agg |
| 25293089 | 1 | atggttgtagaggaggatga | agg |
| 25293100 | 1 | ggaggatgaaggcaacagcc | tgg |
| 25293107 | −1 | gctcttgaatcagtcaagcc | agg |
| 25293121 | 1 | ggcttgactgattcaagagc | agg |
| 25293135 | 1 | aagagcaggagatgagaaag | tgg |
| 25293149 | 1 | agaaagtggagacagcatgc | agg |
| 25293150 | 1 | gaaagtggagacagcatgca | ggg |
| 25293151 | 1 | aaagtggagacagcatgcag | ggg |
| 25293165 | 1 | atgcaggggcagctctgcca | agg |
| 25293171 | −1 | cccctttatagcaaagtcct | tgg |
| 25293180 | 1 | tgccaaggactttgctataa | agg |
| 25293181 | 1 | gccaaggactttgctataaa | ggg |
| 25293182 | 1 | ccaaggactttgctataaag | ggg |
| 25293195 | 1 | tataaaggggaacagagaaa | tgg |
| 25293198 | 1 | aaaggggaacagagaaatgg | agg |
| 25293207 | 1 | cagagaaatggaggagaagc | agg |
| 25293210 | 1 | agaaatggaggagaagcagg | agg |
| 25293211 | 1 | gaaatggaggagaagcagga | ggg |
| 25293230 | 1 | agggcaataatccgatagag | agg |
| 25293230 | −1 | atcagatttttcctctctat | cgg |
| 25293286 | 1 | caagagtcaagcctttgagt | tgg |
| 25293286 | −1 | actcctgctttccaactcaa | agg |
| 25293294 | 1 | aagcctttgagttggaaagc | agg |
| 25293299 | 1 | tttgagttggaaagcaggag | tgg |
| 25293300 | 1 | ttgagttggaaagcaggagt | ggg |
| 25293324 | 1 | ttttgagcactgataccttt | agg |
| 25293328 | −1 | ctgtccctgcatcggcctaa | agg |
| 25293334 | 1 | tgatacctttaggccgatgc | agg |
| 25293335 | 1 | gatacctttaggccgatgca | ggg |
| 25293336 | −1 | aagatgaactgtccctgcat | cgg |
| 25293429 | 1 | CATTAGAGATTCCCATTGTG | CGG |
| 25293429 | −1 | AATTGTTATTTCCGCACAAT | GGG |
| 25293430 | −1 | AAATTGTTATTTCCGCACAA | TGG |
| 25293466 | 1 | TACTTATAGTTTTATATTTG | TGG |
| 25293524 | 1 | AATTAAATCTCAGTTTACAA | TGG |
| 25293547 | 1 | ATAATATTTTGATATGTCTC | TGG |
| 25293548 | 1 | TAATATTTTGATATGTCTCT | GGG |
| 25293549 | 1 | AATATTTTGATATGTCTCTG | GGG |
| 25293567 | 1 | TGGGGAAACTTGCCCTTAAA | TGG |
| 25293568 | −1 | GATACAGAAGTTCCATTTAA | GGG |
| 25293569 | −1 | AGATACAGAAGTTCCATTTA | AGG |
| 25293603 | −1 | AAATCCTAGGAAGAAACGCT | TGG |
| 25293610 | 1 | CACTCCAAGCGTTTCTTCCT | AGG |
| 25293616 | −1 | ATTATAAATTTCTAAATCCT | AGG |
| 25293656 | −1 | ACTAGGGAAATTTTAAAATT | AGG |
| 25293672 | −1 | ACTGATGGTTACATATACTA | GGG |
| 25293673 | −1 | TACTGATGGTTACATATACT | AGG |
| 25293686 | 1 | TAGTATATGTAACCATCAGT | AGG |
| 25293687 | −1 | CAGTAGATACCACCTACTGA | TGG |
| 25293689 | 1 | TATATGTAACCATCAGTAGG | TGG |
| 25293708 | 1 | GTGGTATCTACTGACTAGAG | AGG |
| 25293709 | 1 | TGGTATCTACTGACTAGAGA | GGG |
| 25293787 | −1 | CCTTGGGAATAATGGACAAA | GGG |
| 25293788 | −1 | GCCTTGGGAATAATGGACAA | AGG |
| 25293795 | −1 | CATATTTGCCTTGGGAATAA | TGG |
| 25293798 | 1 | CCCTTTGTCCATTATTCCCA | AGG |
| 25293803 | −1 | CAAATTTCCATATTTGCCTT | GGG |
| 25293804 | −1 | TCAAATTTCCATATTTGCCT | TGG |
| 25293807 | 1 | CATTATTCCCAAGGCAAATA | TGG |
| 25293841 | 1 | TGTACTAATCATAATAAAGC | TGG |
| 25293872 | 1 | TAAGAGATTGAGAAATTAAA | AGG |
| 25293924 | 1 | TTGTGAGTCTTATAAGAAGC | TGG |
| 25293925 | 1 | TGTGAGTCTTATAAGAAGCT | GGG |
| 25293928 | 1 | GAGTCTTATAAGAAGCTGGG | AGG |
| 25293943 | −1 | TGTATTCTGGTGAGTTAATG | GGG |
| 25293944 | −1 | CTGTATTCTGGTGAGTTAAT | GGG |
| 25293945 | −1 | TCTGTATTCTGGTGAGTTAA | TGG |
| 25293956 | −1 | TGAGACTGAGTTCTGTATTC | TGG |
| 25293995 | −1 | CTTTGAGGAAAAGGTTTGAG | AGG |
| 25294004 | −1 | GAATTTAATCTTTGAGGAAA | AGG |
| 25294010 | −1 | TTTTCAGAATTTAATCTTTG | AGG |
| 25294046 | 1 | ATCTTGTGATTAAGAGAAGA | AGG |
| 25294060 | 1 | AGAAGAAGGCTGTCCACCAA | TGG |
| 25294061 | 1 | GAAGAAGGCTGTCCACCAAT | GGG |
| 25294062 | −1 | ATAACAGATAAGCCCATTGG | TGG |
| 25294065 | −1 | GAAATAACAGATAAGCCCAT | TGG |
| 25294090 | −1 | ATGCCATTAAGCTCACAATA | AGG |
| 25294098 | 1 | CTTCCTTATTGTGAGCTTAA | TGG |
| 25294114 | 1 | TTAATGGCATGACAAAGCAG | AGG |
| 25294122 | 1 | ATGACAAAGCAGAGGCAAAG | AGG |
| 25294146 | 1 | ATACATCAATTCTTCAAAGT | AGG |
| 25294158 | 1 | TTCAAAGTAGGAAGTCAAAA | AGG |
| 25294178 | 1 | AGGTCAGAGCTTCCACAGCA | TGG |
| 25294179 | −1 | GCAAAGCTGTTGCCATGCTG | TGG |
| 25294207 | −1 | TATTTCAACTATCACGATGT | GGG |
| 25294208 | −1 | CTATTTCAACTATCACGATG | TGG |
| 25294235 | 1 | GAAATAGCAAAGCCCAGCAA | AGG |
| 25294236 | −1 | TTTCAGCTTTAACCTTTGCT | GGG |
| 25294237 | −1 | TTTTCAGCTTTAACCTTTGC | TGG |
| 25294262 | −1 | AGCTGCCAAGGCAGGGCTTT | TGG |
| 25294268 | 1 | AAATGCCAAAAGCCCTGCCT | TGG |
| 25294269 | −1 | CGCAGAAAGCTGCCAAGGCA | GGG |
| 25294270 | −1 | TCGCAGAAAGCTGCCAAGGC | AGG |
| 25294274 | −1 | TGCCTCGCAGAAAGCTGCCA | AGG |
| 25294283 | 1 | TGCCTTGGCAGCTTTCTGCG | AGG |
| 25294298 | −1 | TGTTACTGATTATGTTCATG | GGG |
| 25294299 | −1 | TTGTTACTGATTATGTTCAT | GGG |
| 25294300 | −1 | GTTGTTACTGATTATGTTCA | TGG |
| 25294321 | 1 | AATCAGTAACAACTTGTCCA | AGG |
| 25294327 | −1 | CTTCATGGTCACTGGGGCCT | TGG |
| 25294333 | −1 | CTCACTCTTCATGGTCACTG | GGG |
| 25294334 | −1 | CCTCACTCTTCATGGTCACT | GGG |
| 25294335 | −1 | CCCTCACTCTTCATGGTCAC | TGG |
| 25294342 | −1 | GCTGCAGCCCTCACTCTTCA | TGG |
| 25294345 | 1 | CCCAGTGACCATGAAGAGTG | AGG |
| 25294346 | 1 | CCAGTGACCATGAAGAGTGA | GGG |
| 25294357 | 1 | GAAGAGTGAGGGCTGCAGCC | AGG |
| 25294358 | 1 | AAGAGTGAGGGCTGCAGCCA | GGG |
| 25294364 | −1 | CTCTGCGACGGACTATTCCC | TGG |
| 25294376 | −1 | TTTGAATCCTTGCTCTGCGA | CGG |
| 25294380 | 1 | GAATAGTCCGTCGCAGAGCA | AGG |
| 25294398 | 1 | CAAGGATTCAAATAAGCAGC | CGG |
| 25294406 | −1 | TTTGCTCCCGGGTCTGCTTC | CGG |
| 25294410 | 1 | TAAGCAGCCGGAAGCAGACC | CGG |
| 25294411 | 1 | AAGCAGCCGGAAGCAGACCC | GGG |
| 25294417 | −1 | GTTGTCAGTGTTTTGCTCCC | GGG |
| 25294418 | −1 | GGTTGTCAGTGTTTTGCTCC | CGG |
| 25294439 | −1 | TCTCCACTGGACTAGCGAGA | GGG |
| 25294440 | −1 | CTCTCCACTGGACTAGCGAG | AGG |
| 25294447 | 1 | CAACCCTCTCGCTAGTCCAG | TGG |
| 25294452 | −1 | CCAAGGCTGCATCTCTCCAC | TGG |
| 25294463 | 1 | CCAGTGGAGAGATGCAGCCT | TGG |
| 25294469 | −1 | GAGCCACCATTCTGGCTCCA | AGG |
| 25294474 | 1 | ATGCAGCCTTGGAGCCAGAA | TGG |
| 25294477 | 1 | CAGCCTTGGAGCCAGAATGG | TGG |
| 25294477 | −1 | TTGTCACCGAGCCACCATTC | TGG |
| 25294482 | 1 | TTGGAGCCAGAATGGTGGCT | CGG |
| 25294516 | −1 | CGACCTATCCCAGAATGGTG | TGG |
| 25294518 | 1 | TGCTGCACTCCACACCATTC | TGG |
| 25294519 | 1 | GCTGCACTCCACACCATTCT | GGG |
| 25294521 | −1 | AGGACCGACCTATCCCAGAA | TGG |
| 25294524 | 1 | ACTCCACACCATTCTGGGAT | AGG |
| 25294528 | 1 | CACACCATTCTGGGATAGGT | CGG |
| 25294541 | −1 | TCATATCTCAGCATTTCTTC | AGG |
| 25294557 | 1 | AGAAATGCTGAGATATGAGC | AGG |
| 25294569 | 1 | ATATGAGCAGGTCTGACCAC | TGG |
| 25294574 | −1 | TCTGTTGCTGCGAACTCCAG | TGG |
| 25294591 | 1 | GAGTTCGCAGCAACAGAGCT | CGG |
| 25294600 | 1 | GCAACAGAGCTCGGCCTCCT | TGG |
| 25294601 | 1 | CAACAGAGCTCGGCCTCCTT | GGG |
| 25294603 | −1 | GCCGTTTGCGGTGCCCAAGG | AGG |
| 25294606 | −1 | AGTGCCGTTTGCGGTGCCCA | AGG |
| 25294613 | 1 | GCCTCCTTGGGCACCGCAAA | CGG |
| 25294615 | −1 | TGGAGGCTGAGTGCCGTTTG | CGG |
| 25294628 | 1 | GCAAACGGCACTCAGCCTCC | AGG |
| 25294629 | 1 | CAAACGGCACTCAGCCTCCA | GGG |
| 25294632 | −1 | ACGAGATGGCGGTTCCCTGG | AGG |
| 25294635 | −1 | GGAACGAGATGGCGGTTCCC | TGG |
| 25294643 | −1 | ccgCCTCAGGAACGAGATGG | CGG |
| 25294646 | −1 | tctccgCCTCAGGAACGAGA | TGG |
| 25294651 | 1 | GAACCGCCATCTCGTTCCTG | AGG |
| 25294654 | 1 | CCGCCATCTCGTTCCTGAGG | cgg |
| 25294656 | −1 | taagatgaactctccgCCTC | AGG |
| 25294680 | 1 | gttcatcttaacgagagaaa | tgg |
| 25294684 | 1 | atcttaacgagagaaatggc | agg |
| 25294685 | 1 | tcttaacgagagaaatggca | ggg |
| 25294697 | 1 | aaatggcagggactgtgaat | agg |
| 25294701 | 1 | ggcagggactgtgaataggc | cgg |
| 25294709 | −1 | gcacccgccaccaaatctgc | cgg |
| 25294710 | 1 | tgtgaataggccggcagatt | tgg |
| 25294713 | 1 | gaataggccggcagatttgg | tgg |
| 25294716 | 1 | taggccggcagatttggtgg | cgg |
| 25294717 | 1 | aggccggcagatttggtggc | ggg |
| 25294726 | 1 | gatttggtggcgggtgccac | agg |
| 25294731 | −1 | cctgcaggagactgaacctg | tgs |
| 25294742 | 1 | ccacaggttcagtctcctgc | agg |
| 25294743 | 1 | cacaggttcagtctcctgca | ggg |
| 25294746 | −1 | gcattttctcctctccctgc | agg |
| 25294748 | 1 | gttcagtctcctgcagggag | agg |
| 25294768 | −1 | gaaaatacaaggaattagta | agg |
| 25294779 | −1 | tgtttctctgagaaaataca | agg |
| 25294795 | 1 | tattttctcagagaaacaag | agg |
| 25294809 | −1 | ccctcacatgaggctgatga | cgg |
| 25294819 | 1 | accgtcatcagcctcatgtg | agg |
| 25294819 | −1 | ctccttcccaccctcacatg | agg |
| 25294820 | 1 | ccgtcatcagcctcatgtga | ggg |
| 25294823 | 1 | tcatcagcctcatgtgaggg | tgg |
| 25294824 | 1 | catcagcctcatgtgagggt | ggg |
| 25294828 | 1 | agcctcatgtgagggtggga | agg |
| 25294831 | 1 | ctcatgtgagggtgggaagg | agg |
| 25294832 | 1 | tcatgtgagggtgggaagga | ggg |
| 25294836 | 1 | gtgagggtgggaaggaggga | tgg |
| 25294837 | 1 | tgagggtgggaaggagggat | ggg |
| 25294838 | 1 | gagggtgggaaggagggatg | ggg |
| 25294845 | 1 | ggaaggagggatggggtttg | cgg |
| 25294850 | 1 | gagggatggggtttgcggag | agg |
| 25294851 | 1 | agggatggggtttgcggaga | ggg |
| 25294860 | 1 | gtttgcggagagggaaagtg | tgg |
| 25294865 | 1 | cggagagggaaagtgtggta | tgg |
| 25294875 | 1 | aagtgtggtatggtcatctg | tgg |
| 25294876 | 1 | agtgtggtatggtcatctgt | ggg |
| 25294881 | 1 | ggtatggtcatctgtgggag | tgg |
| 25294895 | 1 | tgggagtggaagagagtgag | agg |
| 25294896 | 1 | gggagtggaagagagtgaga | ggg |
| 25294903 | 1 | gaagagagtgagagggctgc | agg |
| 25294904 | 1 | aagagagtgagagggctgca | ggg |
| 25294905 | 1 | agagagtgagagggctgcag | ggg |
| 25294913 | 1 | agagggctgcaggggtgcag | cgg |
| 25294914 | 1 | gagggctgcaggggtgcagc | ggg |
| 25294922 | 1 | caggggtgcagcgggactgc | agg |
| 25294926 | 1 | ggtgcagcgggactgcaggc | tgg |
| 25294933 | 1 | cgggactgcaggctggcacc | agg |
| 25294934 | 1 | gggactgcaggctggcacca | ggg |
| 25294940 | −1 | actacaagccctagggaccc | tgg |
| 25294942 | 1 | aggctggcaccagggtccct | agg |
| 25294943 | 1 | ggctggcaccagggtcccta | ggg |
| 25294947 | −1 | tccaccaactacaagcccta | ggg |
| 25294948 | −1 | ttccaccaactacaagccct | agg |
| 25294954 | 1 | gggtccctagggcttgtagt | tgg |
| 25294957 | 1 | tccctagggcttgtagttgg | tgg |
| 25294977 | 1 | tggaaagtgcatcagtgacc | agg |
| 25294978 | 1 | ggaaagtgcatcagtgacca | ggg |
| 25294984 | −1 | ggagcagctgcacacagccc | tgg |
| 25294998 | 1 | gggctgtgtgcagctgctcc | agg |
| 25295002 | 1 | tgtgtgcagctgctccaggc | agg |
| 25295005 | −1 | ctgcttcttccacacctgcc | tgg |
| 25295007 | 1 | gcagctgctccaggcaggtg | tgg |
| 25295037 | 1 | agagttgaacttgcccagcc | tgg |
| 25295039 | −1 | tctgggcagcactccaggct | ggg |
| 25295040 | −1 | ctctgggcagcactccaggc | tgg |
| 25295044 | −1 | ctcactctgggcagcactcc | agg |
| 25295056 | −1 | ctgggctttgggctcactct | ggg |
| 25295057 | −1 | cctgggctttgggctcactc | tgg |
| 25295067 | −1 | tctggtctcccctgggcttt | ggg |
| 25295068 | 1 | ccagagtgagcccaaagccc | agg |
| 25295068 | −1 | ctctggtctcccctgggctt | tgg |
| 25295069 | 1 | cagagtgagcccaaagccca | ggg |
| 25295070 | 1 | agagtgagcccaaagcccag | ggg |
| 25295074 | −1 | ccccatctctggtctcccct | ggg |
| 25295075 | −1 | gccccatctctggtctcccc | tgg |
| 25295083 | 1 | agcccaggggagaccagaga | tgg |
| 25295084 | 1 | gcccaggggagaccagagat | ggg |
| 25295085 | 1 | cccaggggagaccagagatg | ggg |
| 25295085 | −1 | tttgcaaacagccccatctc | tgg |
| 25295098 | 1 | agagatggggctgtttgcaa | agg |
| 25295101 | 1 | gatggggctgtttgcaaagg | agg |
| 25295127 | −1 | ttaaccagctcagattttgt | ggg |
| 25295128 | −1 | cttaaccagctcagattttg | tgg |
| 25295134 | 1 | gtagcccacaaaatctgagc | tgg |
| 25295144 | 1 | aaatctgagctggttaagaa | agg |
| 25295161 | 1 | gaaaggagagagagTGAAAA | TGG |
| 25295162 | 1 | aaaggagagagagTGAAAAT | GGG |
| 25295163 | 1 | aaggagagagagTGAAAATG | GGG |
| 25295175 | 1 | TGAAAATGGGGAGCCCagcc | tgg |
| 25295177 | −1 | tgtacccaggctgccaggct | GGG |
| 25295178 | −1 | gtgtacccaggctgccaggc | tGG |
| 25295182 | −1 | agatgtgtacccaggctgcc | agg |
| 25295183 | 1 | GGGAGCCCagcctggcagcc | tgg |
| 25295184 | 1 | GGAGCCCagcctggcagcct | ggg |
| 25295190 | −1 | ttgagctgagatgtgtaccc | agg |
| 25295213 | −1 | caaatggattcagctagtgt | ggg |
| 25295214 | −1 | ccaaatggattcagctagtg | tgg |
| 25295225 | 1 | ccacactagctgaatccatt | tgg |
| 25295226 | 1 | cacactagctgaatccattt | ggg |
| 25295229 | −1 | ggtcaacgaaggggcccaaa | tgg |
| 25295238 | −1 | gcacagagaggtcaacgaag | ggg |
| 25295239 | −1 | ggcacagagaggtcaacgaa | ggg |
| 25295240 | −1 | aggcacagagaggtcaacga | agg |
| 25295250 | −1 | agggaaactgaggcacagag | agg |
| 25295260 | −1 | ttctatagatagggaaactg | agg |
| 25295269 | −1 | ttatccccattctatagata | ggg |
| 25295270 | −1 | cttatccccattctatagat | agg |
| 25295274 | 1 | cagtttccctatctatagaa | tgg |
| 25295275 | 1 | agtttccctatctatagaat | ggg |
| 25295276 | 1 | gtttccctatctatagaatg | ggg |
| 25295288 | 1 | atagaatggggataagaata | agg |
| 25295300 | 1 | taagaataaggctacttcct | agg |
| 25295301 | 1 | aagaataaggctacttccta | ggg |
| 25295306 | −1 | tcaatcctcacaacagccct | agg |
| 25295312 | 1 | tacttcctagggctgttgtg | agg |
| 25295337 | −1 | ttcaaaattgaacaagtgtt | cgg |
| 25295369 | 1 | aacactgttctaaagcattt | agg |
| 25295380 | 1 | aaagcatttaggacagtgcc | tgg |
| 25295385 | 1 | atttaggacagtgcctggca | tgg |
| 25295386 | 1 | tttaggacagtgcctggcat | ggg |
| 25295387 | 1 | ttaggacagtgcctggcatg | ggg |
| 25295387 | −1 | CGCaacacttaccccatgcc | agg |
| 25295399 | 1 | ctggcatggggtaagtgttG | CGG |
| 25295434 | −1 | TCAACGCAGCCTGAGAACAA | TGG |
| 25295436 | 1 | TCATCATCACCATTGTTCTC | AGG |
| 25295449 | 1 | TGTTCTCAGGCTGCGTTGAT | TGg |
| 25295461 | 1 | GCGTTGATTGgagctgctga | agg |
| 25295462 | 1 | CGTTGATTGgagctgctgaa | ggg |
| 25295465 | 1 | TGATTGgagctgctgaaggg | agg |
| 25295475 | 1 | tgctgaagggaggcaattta | agg |
| 25295486 | 1 | ggcaatttaaggaagtgagc | cgg |
| 25295494 | −1 | accaccacctcctatctgtc | cgg |
| 25295495 | 1 | aggaagtgagccggacagat | agg |
| 25295498 | 1 | aagtgagccggacagatagg | agg |
| 25295501 | 1 | tgagccggacagataggagg | tgg |
| 25295504 | 1 | gccggacagataggaggtgg | tgg |
| 25295507 | 1 | ggacagataggaggtggtgg | tgg |
| 25295515 | 1 | aggaggtggtggtggttatc | agg |
| 25295535 | 1 | aggtgcgatgcttgaaactg | agg |
| 25295541 | 1 | gatgcttgaaactgaggctt | cgg |
| 25295544 | 1 | gcttgaaactgaggcttcgg | agg |
| 25295557 | 1 | gcttcggaggcaacagttac | tgg |
| 25295568 | 1 | aacagttactggtaatgaca | agg |
| 25295575 | 1 | actggtaatgacaaggtcta | agg |
| 25295586 | 1 | caaggtctaaggcttgacag | tgg |
| 25295587 | 1 | aaggtctaaggcttgacagt | ggg |
| 25295590 | 1 | gtctaaggcttgacagtggg | tgg |
| 25295607 | 1 | gggtggcagaagtgtaacgc | agg |
| 25295608 | 1 | ggtggcagaagtgtaacgca | ggg |
| 25295622 | 1 | aacgcagggaaagagacgag | cgg |
| 25295628 | 1 | gggaaagagacgagcggtca | agg |
| 25295638 | 1 | cgagcggtcaaggagccgag | agg |
| 25295639 | 1 | gagcggtcaaggagccgaga | ggg |
| 25295642 | −1 | ccacccaactccttccctct | cgg |
| 25295643 | 1 | ggtcaaggagccgagaggga | agg |
| 25295649 | 1 | ggagccgagagggaaggagt | tgg |
| 25295650 | 1 | gagccgagagggaaggagtt | ggg |
| 25295653 | 1 | ccgagagggaaggagttggg | tgg |
| 25295670 | 1 | gggtggactaagatcatttg | tgg |
| 25295681 | 1 | gatcatttgtggaagaatga | tgg |
| 25295690 | 1 | tggaagaatgatggagagaa | agg |
| 25295697 | 1 | atgatggagagaaaggctga | agg |
| 25295698 | 1 | tgatggagagaaaggctgaa | ggg |
| 25295702 | 1 | ggagagaaaggctgaagggc | agg |
| 25295703 | 1 | gagagaaaggctgaagggca | ggg |
| 25295704 | 1 | agagaaaggctgaagggcag | ggg |
| 25295728 | 1 | tgacatcatcagtgaccaag | agg |
| 25295731 | 1 | catcatcagtgaccaagagg | cgg |
| 25295732 | −1 | tcagcctcccggccgcctct | tgg |
| 25295735 | 1 | atcagtgaccaagaggcggc | cgg |
| 25295736 | 1 | tcagtgaccaagaggcggcc | ggg |
| 25295739 | 1 | gtgaccaagaggcggccggg | agg |
| 25295743 | −1 | ttgctgtggtctcagcctcc | cgg |
| 25295757 | −1 | acactctccctttcttgctg | tgg |
| 25295760 | 1 | ggctgagaccacagcaagaa | agg |
| 25295761 | 1 | gctgagaccacagcaagaaa | ggg |
| 25295773 | 1 | gcaagaaagggagagtgtga | tgg |
| 25295787 | 1 | gtgtgatggcatcttcttca | agg |
| 25295788 | 1 | tgtgatggcatcttcttcaa | ggg |
| 25295794 | 1 | ggcatcttcttcaagggagc | tgg |
| 25295795 | 1 | gcatcttcttcaagggagct | ggg |
| 25295796 | 1 | catcttcttcaagggagctg | ggg |
| 25295804 | 1 | tcaagggagctggggatgtt | tgg |
| 25295805 | 1 | caagggagctggggatgttt | ggg |
| 25295806 | 1 | aagggagctggggatgtttg | ggs |
| 25295809 | 1 | ggagctggggatgtttgggg | tgg |
| 25295824 | 1 | tggggtggaaaaaagaacaa | tgg |
| 25295829 | 1 | tggaaaaaagaacaatggtc | tgg |
| 25295830 | 1 | ggaaaaaagaacaatggtct | ggg |
| 25295833 | 1 | aaaaagaacaatggtctggg | agG |
| 25295834 | 1 | aaaagaacaatggtctggga | gGG |
| 25295841 | 1 | caatggtctgggagGGAATA | TGG |
| 25295842 | 1 | aatggtctgggagGGAATAT | GGG |
| 25295881 | 1 | ttttttttttttttttgaga | tgg |
| 25295903 | 1 | gagtttcgctgttgtcatcc | agg |
| 25295907 | 1 | ttcgctgttgtcatccaggc | tgg |
| 25295910 | −1 | tgcaacattgcaatccagcc | tgg |
| 25295928 | 1 | ggattgcaatgttgcaatct | tgg |
| 25295953 | 1 | cactgcaacttctgccttcc | agg |
| 25295956 | −1 | gagaatcacttgaacctgga | agg |
| 25295960 | −1 | acaggagaatcacttgaacc | tgg |
| 25295978 | −1 | gctactcgggaagctgagac | agg |
| 25295991 | −1 | gcctgtaatctcagctactc | ggg |
| 25295992 | −1 | tgcctgtaatctcagctact | cgg |
| 25296001 | 1 | tcccgagtagctgagattac | agg |
| 25296019 | −1 | caaaagtaagccaggcgtgg | tgg |
| 25296020 | 1 | caggcacacaccaccacgcc | tgg |
| 25296022 | −1 | atacaaaagtaagccaggcg | tgg |
| 25296027 | −1 | taaaaatacaaaagtaagcc | agg |
| 25296048 | 1 | ttttgtatttttagtagaga | cgg |
| 25296064 | 1 | gagacggagttttgccatgt | tgg |
| 25296067 | −1 | tgagaccagcctggccaaca | tgg |
| 25296069 | 1 | ggagttttgccatgttggcc | agg |
| 25296073 | 1 | ttttgccatgttggccaggc | tgg |
| 25296076 | −1 | tcaggagtttgagaccagcc | tgg |
| 25296094 | 1 | ggtctcaaactcctgacctc | agg |
| 25296094 | −1 | cgggtggatcacctgaggtc | agg |
| 25296099 | −1 | caaggcgggtggatcacctg | agg |
| 25296110 | −1 | ctttgggaggccaaggcggg | tgg |
| 25296111 | 1 | ctcaggtgatccacccgcct | tgg |
| 25296113 | −1 | gcactttgggaggccaaggc | ggg |
| 25296114 | −1 | agcactttgggaggccaagg | cgg |
| 25296117 | −1 | cccagcactttgggaggcca | agg |
| 25296123 | −1 | tctaatcccagcactttggg | agg |
| 25296126 | −1 | acctctaatcccagcacttt | ggg |
| 25296127 | 1 | gccttggcctcccaaagtgc | tgg |
| 25296127 | −1 | cacctctaatcccagcactt | tgg |
| 25296128 | 1 | ccttggcctcccaaagtgct | ggg |
| 25296136 | 1 | tcccaaagtgctgggattag | agg |
| 25296154 | −1 | AACTTCCAggctgggcgcgg | tgg |
| 25296157 | −1 | ACAAACTTCCAggctgggcg | cgg |
| 25296160 | 1 | gtgagccaccgcgcccagcc | TGG |
| 25296162 | −1 | TAAATACAAACTTCCAggct | ggg |
| 25296163 | −1 | ATAAATACAAACTTCCAggc | tgg |
| 25296167 | −1 | ATTAATAAATACAAACTTCC | Agg |
| 25296184 | 1 | AGTTTGTATTTATTAATTTT | TGG |
| 25296224 | −1 | atgtacactgaagtatttag | ggg |
| 25296225 | −1 | aatgtacactgaagtattta | ggg |
| 25296226 | −1 | aaatgtacactgaagtattt | agg |
| 25296283 | −1 | actccagcctgggtgattga | tgg |
| 25296287 | 1 | tcttgctccatcaatcaccc | agg |
| 25296291 | 1 | gctccatcaatcacccaggc | tgg |
| 25296293 | −1 | acaccaccgcactccagcct | ggg |
| 25296294 | −1 | cacaccaccgcactccagcc | tgg |
| 25296298 | 1 | caatcacccaggctggagtg | cgg |
| 25296301 | 1 | tcacccaggctggagtgcgg | tgg |
| 25296312 | 1 | ggagtgcggtggtgtgatct | cgg |
| 25296334 | −1 | tgcttgaatccaggaggcgg | agg |
| 25296336 | 1 | tcactgcaacctccgcctcc | tgg |
| 25296337 | −1 | aattgcttgaatccaggagg | cgg |
| 25296340 | −1 | aagaattgcttgaatccagg | agg |
| 25296343 | −1 | cacaagaattgcttgaatcc | agg |
| 25296366 | −1 | cccagctactcgggagggtg | agg |
| 25296371 | −1 | ctaatcccagctactcggga | ggg |
| 25296372 | −1 | cctaatcccagctactcggg | agg |
| 25296375 | −1 | gcccctaatcccagctactc | ggg |
| 25296376 | 1 | gcctcaccccccgagtagc | tgg |
| 25296376 | −1 | tgcccctaatcccagctact | cgg |
| 25296377 | 1 | cctcaccctcccgagtagct | ggg |
| 25296383 | 1 | cctcccgagtagctgggatt | agg |
| 25296384 | 1 | ctcccgagtagctgggatta | ggg |
| 25296385 | 1 | tcccgagtagctgggattag | ggg |
| 25296401 | −1 | caaaaattaactgggcatgg | tgg |
| 25296404 | −1 | atacaaaaattaactgggca | tgg |
| 25296409 | −1 | taaaaatacaaaaattaact | ggg |
| 25296410 | −1 | ctaaaaatacaaaaattaac | tgg |
| 25296430 | 1 | ttttgtatttttagtagaga | tgg |
| 25296446 | 1 | gagatggagtttcaccatat | tgg |
| 25296449 | −1 | caagaccagcctggccaata | tgg |
| 25296451 | 1 | ggagtttcaccatattggcc | agg |
| 25296455 | 1 | tttcaccatattggccaggc | tgg |
| 25296458 | −1 | ccaggagctcaagaccagcc | tgg |
| 25296469 | 1 | ccaggctggtcttgagctcc | tgg |
| 25296476 | −1 | caggtggatcaactgaggcc | agg |
| 25296481 | −1 | tgagacaggtggatcaactg | agg |
| 25296492 | −1 | atttgggaggctgagacagg | tgg |
| 25296495 | −1 | gcaatttgggaggctgagac | agg |
| 25296505 | −1 | tgtaatctcagcaatttggg | agg |
| 25296508 | −1 | gcctgtaatctcagcaattt | ggg |
| 25296509 | −1 | cgcctgtaatctcagcaatt | tgg |
| 25296518 | 1 | tcccaaattgctgagattac | agg |
| 25296523 | 1 | aattgctgagattacaggcg | tgg |
| 25296524 | 1 | attgctgagattacaggcgt | ggg |
| 25296536 | −1 | tacactgaggccggttatgg | tgg |
| 25296537 | 1 | caggcgtgggccaccataac | cgg |
| 25296539 | −1 | atatacactgaggccggtta | tgg |
| 25296545 | −1 | tcagaaatatacactgaggc | cgg |
| 25296549 | −1 | tgcatcagaaatatacactg | agg |
| 25296565 | 1 | gtgtatatttctgatgcagt | tgg |
| 25296566 | 1 | tgtatatttctgatgcagtt | ggg |
| 25296586 | −1 | attcgagatgagattggagg | ggg |
| 25296587 | −1 | aattcgagatgagattggag | ggg |
| 25296588 | −1 | caattcgagatgagattgga | ggg |
| 25296589 | −1 | acaattcgagatgagattgg | agg |
| 25296592 | −1 | attacaattcgagatgagat | tgg |
| 25296615 | −1 | ggtcatgccctcaacacgtg | ggg |
| 25296616 | −1 | aggtcatgccctcaacacgt | ggg |
| 25296617 | −1 | gaggtcatgccctcaacacg | tgg |
| 25296618 | 1 | attgtaatccccacgtgttg | agg |
| 25296619 | 1 | ttgtaatccccacgtgttga | ggg |
| 25296632 | 1 | gtgttgagggcatgacctcg | tgg |
| 25296633 | 1 | tgttgagggcatgacctcgt | ggg |
| 25296636 | 1 | tgagggcatgacctcgtggg | agg |
| 25296636 | −1 | tgatccaatcacctcccacg | agg |
| 25296643 | 1 | atgacctcgtgggaggtgat | tgg |
| 25296651 | 1 | gtgggaggtgattggatcac | agg |
| 25296652 | 1 | tgggaggtgattggatcaca | ggg |
| 25296653 | 1 | gggaggtgattggatcacag | ggg |
| 25296656 | 1 | aggtgattggatcacagggg | tgg |
| 25296671 | −1 | ctgtcacaagaacagcatgg | ggg |
| 25296672 | −1 | actgtcacaagaacagcatg | ggg |
| 25296673 | −1 | cactgtcacaagaacagcat | ggg |
| 25296674 | −1 | tcactgtcacaagaacagca | tgg |
| 25296689 | 1 | gctgttcttgtgacagtgag | tgg |
| 25296690 | 1 | ctgttcttgtgacagtgagt | ggg |
| 25296698 | 1 | gtgacagtgagtgggttttc | agg |
| 25296709 | 1 | tgggttttcaggagagctga | tgg |
| 25296722 | 1 | gagctgatggtttgaaagtg | tgg |
| 25296739 | −1 | agagagagagaaagagagag | agg |
| 25296766 | −1 | ggcacatcttacgtggtgtc | agg |
| 25296773 | −1 | gaagcaaggcacatcttacg | tgg |
| 25296787 | −1 | tggaaggtgaaagggaagca | agg |
| 25296795 | −1 | aatcatggtggaaggtgaaa | ggg |
| 25296796 | −1 | caatcatggtggaaggtgaa | agg |
| 25296803 | −1 | aaacttacaatcatggtgga | agg |
| 25296807 | −1 | caggaaacttacaatcatgg | tgg |
| 25296810 | −1 | cctcaggaaacttacaatca | tgg |
| 25296821 | 1 | ccatgattgtaagtttcctg | agg |
| 25296826 | −1 | ggcatggccggggaggcctc | agg |
| 25296830 | 1 | taagtttcctgaggcctccc | cgg |
| 25296833 | −1 | acagtttggcatggccgggg | agg |
| 25296836 | −1 | ctcacagtttggcatggccg | ggg |
| 25296837 | −1 | actcacagtttggcatggcc | ggg |
| 25296838 | −1 | gactcacagtttggcatggc | cgg |
| 25296842 | −1 | aattgactcacagtttggca | tgg |
| 25296847 | −1 | ggctgaattgactcacagtt | tgg |
| 25296868 | −1 | gcgtaatttataaacaaaag | agg |
| 25296888 | 1 | tttataaattacgcagtctc | agg |
| 25296941 | 1 | taacacaatttcctaaaaca | agg |
| 25296941 | −1 | agagaatgtccccttgtttt | agg |
| 25296942 | 1 | aacacaatttcctaaaacaa | ggg |
| 25296943 | 1 | acacaatttcctaaaacaag | ggg |
| 25296971 | −1 | catttttgttaactgaaaaa | agg |
| 25297022 | −1 | aaattggtgaaatgagaata | agg |
| 25297038 | −1 | aaagatattattgagaaaat | tgg |
| 25297077 | 1 | aaaaaaatatatattttttg | tgg |
| 25297083 | 1 | atatatattttttgtggtcg | agg |
| 25297133 | 1 | cttattaaattccatcaatc | tgg |
| 25297133 | −1 | aagaaactgctccagattga | tgg |
| 25297178 | −1 | cgaaacttcaaaacatgtca | ags |
| 25297203 | −1 | cccacattctacaaaagaac | tgg |
| 25297213 | 1 | gccagttcttttgtagaatg | tgg |
| 25297214 | 1 | ccagttcttttgtagaatgt | ggg |
| 25297239 | −1 | atacccacaatctaatcatg | agg |
| 25297246 | 1 | tgttcctcatgattagattg | tgg |
| 25297247 | 1 | gttcctcatgattagattgt | ggg |
| 25297260 | 1 | agattgtgggtatgcatttt | tgg |
| 25297264 | 1 | tgtgggtatgcatttttggt | agg |
| 25297282 | −1 | agaagggcacacacggctct | tgg |
| 25297289 | −1 | tatactaagaagggcacaca | cgg |
| 25297298 | −1 | ctgatatgatatactaagaa | ggg |
| 25297299 | −1 | tctgatatgatatactaaga | agg |
| 25297340 | 1 | ctatcaatttgccccattac | tgg |
| 25297340 | −1 | agttaacacacccagtaatg | ggg |
| 25297341 | 1 | tatcaatttgccccattact | ggg |
| 25297341 | −1 | cagttaacacacccagtaat | ggg |
| 25297342 | −1 | acagttaacacacccagtaa | tgg |
| 25297362 | 1 | ggtgtgttaactgtgatcat | tgg |
| 25297363 | 1 | gtgtgttaactgtgatcatt | ggg |
| 25297372 | 1 | ctgtgatcattgggttaaga | tgg |
| 25297383 | 1 | gggttaagatggtacctgcc | agg |
| 25297400 | −1 | ggaaaatagtaactttgcag | tgg |
| 25297421 | −1 | gatgtttattaattacaaag | ggg |
| 25297422 | −1 | agatgtttattaattacaaa | ggg |
| 25297423 | −1 | aagatgtttattaattacaa | agg |
| 25297440 | 1 | taattaataaacatcttgtg | agg |
| 25297461 | −1 | atgatcaacaggatttctat | agg |
| 25297472 | −1 | gtgaaagttggatgatcaac | agg |
| 25297484 | −1 | taaaatcagtgggtgaaagt | tgg |
| 25297494 | −1 | caatgaacactaaaatcagt | ggg |
| 25297495 | −1 | tcaatgaacactaaaatcag | tgg |
| 25297523 | −1 | ttatagtactaatttattca | ggg |
| 25297524 | −1 | attatagtactaatttattc | agg |
| 25297547 | 1 | agtactataataattgccaa | tgg |
| 25297550 | 1 | actataataattgccaatgg | tgg |
| 25297552 | −1 | tggaattagaaaaccaccat | tgg |
| 25297572 | −1 | gccaactactgaaggaaaga | tgg |
| 25297580 | −1 | agaagaatgccaactactga | agg |
| 25297582 | 1 | tccatctttccttcagtagt | tgg |
| 25297597 | 1 | gtagttggcattcttctgta | agg |
| 25297633 | −1 | taaataagtacatagatgag | tgg |
| 25297655 | 1 | tgtacttatttatatcacca | tgg |
| 25297656 | 1 | gtacttatttatatcaccat | ggg |
| 25297661 | −1 | AACCGgaatccaggagccca | tgg |
| 25297663 | 1 | tttatatcaccatgggctcc | tgg |
| 25297670 | 1 | caccatgggctcctggattc | CGG |
| 25297670 | −1 | AAGTGTGTAAACCGgaatcc | agg |
| 25297678 | −1 | GAAAATGGAAGTGTGTAAAC | CGg |
| 25297693 | −1 | CAGAGAGAAAAGGCAGAAAA | TGG |
| 25297703 | −1 | TTATATTAAGCAGAGAGAAA | AGG |
| 25297716 | 1 | TTTTCTCTCTGCTTAATATA | AGG |
| 25297741 | −1 | CATTTTCTTCCTGGGAATCA | GGG |
| 25297742 | −1 | ACATTTTCTTCCTGGGAATC | AGG |
| 25297743 | 1 | ATGAGAACTCCCTGATTCCC | AGG |
| 25297749 | −1 | TCTGCTGACATTTTCTTCCT | GGG |
| 25297750 | −1 | CTCTGCTGACATTTTCTTCC | TGG |
| 25297771 | 1 | AATGTCAGCAGAGCTTTCTT | AGG |
| 25297774 | 1 | GTCAGCAGAGCTTTCTTAGG | CGG |
| 25297807 | 1 | ATTCAGTGTAAGAACCATAA | AGG |
| 25297810 | −1 | ACTACACAGATACACCTTTA | TGG |
| 25297825 | 1 | AAAGGTGTATCTGTGTAGTA | TGG |
| 25297865 | 1 | ACAAACACAAAGAACCTCCA | AGG |
| 25297866 | 1 | CAAACACAAAGAACCTCCAA | GGG |
| 25297868 | −1 | GCAGCACCTCCTGCCCTTGG | AGG |
| 25297870 | 1 | CACAAAGAACCTCCAAGGGC | AGG |
| 25297871 | −1 | CTGGCAGCACCTCCTGCCCT | TGG |
| 25297873 | 1 | AAAGAACCTCCAAGGGCAGG | AGG |
| 25297889 | 1 | CAGGAGGTGCTGCCAGACTC | AGG |
| 25297890 | −1 | TTCTAGTGCCCTCCTGAGTC | TGG |
| 25297892 | 1 | GAGGTGCTGCCAGACTCAGG | AGG |
| 25297893 | 1 | AGGTGCTGCCAGACTCAGGA | GGG |
| 25297905 | 1 | ACTCAGGAGGGCACTAGAAC | TGG |
| 25297927 | −1 | CAGACTACCTGGGATCTCAG | TGG |
| 25297931 | 1 | TGAGAAGCCACTGAGATCCC | AGG |
| 25297937 | −1 | ATGGAGAGCACAGACTACCT | GGG |
| 25297938 | −1 | GATGGAGAGCACAGACTACC | TGG |
| 25297955 | 1 | AGTCTGTGCTCTCCATCTTT | TGG |
| 25297956 | −1 | AGAGAATAAGAGCCAAAAGA | TGG |
| 25297979 | −1 | GTACAGAGATGTTAGATGTA | CGG |
| 25298003 | −1 | TTTTTCGCTAAAGAGAAAGC | TGG |
| 25298031 | −1 | AGGTGGATGGGTGGGTGGAG | GGG |
| 25298032 | −1 | GAGGTGGATGGGTGGGTGGA | GGG |
| 25298033 | −1 | GGAGGTGGATGGGTGGGTGG | AGG |
| 25298036 | −1 | AGTGGAGGTGGATGGGTGGG | TGG |
| 25298039 | −1 | ACAAGTGGAGGTGGATGGGT | GGG |
| 25298040 | −1 | AACAAGTGGAGGTGGATGGG | TGG |
| 25298043 | −1 | AGGAACAAGTGGAGGTGGAT | GGG |
| 25298044 | −1 | CAGGAACAAGTGGAGGTGGA | TGG |
| 25298048 | −1 | AATGCAGGAACAAGTGGAGG | TGG |
| 25298051 | −1 | AGAAATGCAGGAACAAGTGG | AGG |
| 25298054 | −1 | CATAGAAATGCAGGAACAAG | TGG |
| 25298063 | −1 | GATCTGGGACATAGAAATGC | AGG |
| 25298078 | −1 | AGTTGTTTTCTGCAGGATCT | GGG |
| 25298079 | −1 | GAGTTGTTTTCTGCAGGATC | TGG |
| 25298085 | −1 | AGAAAAGAGTTGTTTTCTGC | AGG |
| 25298125 | 1 | tagtctcaattctgtagtcc | agg |
| 25298126 | 1 | agtctcaattctgtagtcca | ggg |
| 25298132 | −1 | ctgatcagattctctctccc | tgg |
| 25298151 | 1 | agagaatctgatcagtcccc | tgg |
| 25298152 | 1 | gagaatctgatcagtcccct | ggg |
| 25298156 | −1 | agagtggaaaaatgacccag | ggg |
| 25298157 | −1 | cagagtggaaaaatgaccca | ggg |
| 25298158 | −1 | ccagagtggaaaaatgaccc | agg |
| 25298169 | 1 | cctgggtcatttttccactc | tgg |
| 25298172 | −1 | tgtagctgcttggaccagag | tgg |
| 25298182 | −1 | ccatgccagctgtagctgct | tgg |
| 25298188 | 1 | ctggtccaagcagctacagc | tgg |
| 25298193 | 1 | ccaagcagctacagctggca | tgg |
| 25298194 | 1 | caagcagctacagctggcat | ggg |
| 25298220 | 1 | tagttcacacagtaaaaaca | tgg |
| 25298234 | 1 | aaaacatggctgtcaagAAG | AGG |
| 25298249 | 1 | agAAGAGGAGTAAATTTCAG | AGG |
| 25298270 | −1 | GGAAGAGGTTCGGGCTCACA | GGG |
| 25298271 | −1 | AGGAAGAGGTTCGGGCTCAC | AGG |
| 25298279 | −1 | AACAAAGCAGGAAGAGGTTC | GGG |
| 25298280 | −1 | CAACAAAGCAGGAAGAGGTT | CGG |
| 25298285 | −1 | GACTGCAACAAAGCAGGAAG | AGG |
| 25298291 | −1 | TATGAAGACTGCAACAAAGC | AGG |
| 25298377 | 1 | CTTTGACTTGCTAGCTTAAC | TGG |
| 25298385 | 1 | TGCTAGCTTAACTGGTCTAG | AGG |
| 25298388 | 1 | TAGCTTAACTGGTCTAGAGG | AGG |
| 25298389 | 1 | AGCTTAACTGGTCTAGAGGA | GGG |
| 25298429 | −1 | CAGGCTGAATTGAAGTTTTG | AGG |
| 25298441 | 1 | CTCAAAACTTCAATTCAGCC | TGG |
| 25298442 | 1 | TCAAAACTTCAATTCAGCCT | GGG |
| 25298448 | −1 | CCCTCCTGCTGAAGAAACCC | AGG |
| 25298455 | 1 | TCAGCCTGGGTTTCTTCAGC | AGG |
| 25298458 | 1 | GCCTGGGTTTCTTCAGCAGG | AGG |
| 25298459 | 1 | CCTGGGTTTCTTCAGCAGGA | GGG |
| 25298464 | 1 | GTTTCTTCAGCAGGAGGGCC | CGG |
| 25298465 | 1 | TTTCTTCAGCAGGAGGGCCC | GGG |
| 25298466 | 1 | TTCTTCAGCAGGAGGGCCCG | GGG |
| 25298467 | 1 | TCTTCAGCAGGAGGGCCCGG | GGG |
| 25298471 | −1 | TCCCTGGCTCTGGTTCCCCC | GGG |
| 25298472 | −1 | GTCCCTGGCTCTGGTTCCCC | CGG |
| 25298480 | 1 | GGCCCGGGGGAACCAGAGCC | AGG |
| 25298481 | 1 | GCCCGGGGGAACCAGAGCCA | GGG |
| 25298481 | −1 | ATGACTCTGGTCCCTGGCTC | TGG |
| 25298487 | −1 | ACTGAAATGACTCTGGTCCC | TGG |
| 25298494 | −1 | CTGGTGCACTGAAATGACTC | TGG |
| 25298513 | −1 | GGAATATTCATTTCTTGAGC | TGG |
| 25298527 | 1 | GCTCAAGAAATGAATATTCC | AGG |
| 25298534 | −1 | ACACTTGGGGATTCTTGGCC | TGG |
| 25298539 | −1 | GAAGAACACTTGGGGATTCT | TGG |
| 25298547 | −1 | GAGTTCAGGAAGAACACTTG | GGG |
| 25298548 | −1 | gGAGTTCAGGAAGAACACTT | GGG |
| 25298549 | −1 | agGAGTTCAGGAAGAACACT | TGG |
| 25298561 | −1 | actccaccaggaagGAGTTC | AGG |
| 25298566 | 1 | GTTCTTCCTGAACTCcttcc | tgg |
| 25298569 | 1 | CTTCCTGAACTCcttcctgg | tgg |
| 25298569 | −1 | ctctttgaactccaccagga | agG |
| 25298573 | −1 | tcatctctttgaactccacc | agg |
| 25298606 | −1 | cctgataagaactgaaaagc | ggg |
| 25298607 | −1 | tcctgataagaactgaaaag | cgg |
| 25298617 | 1 | cccgcttttcagttcttatc | agg |
| 25298645 | −1 | gccctcagtcatacataaag | agg |
| 25298654 | 1 | ttcctctttatgtatgactg | agg |
| 25298655 | 1 | tcctctttatgtatgactga | ggg |
| 25298674 | −1 | tttgtgaagggaacaaatGA | tgg |
| 25298686 | −1 | accaaataaatatttgtgaa | ggg |
| 25298687 | −1 | taccaaataaatatttgtga | agg |
| 25298696 | 1 | tcccttcacaaatatttatt | tgg |
| 25298714 | 1 | tttggtatttactatatacc | agg |
| 25298715 | 1 | ttggtatttactatatacca | ggg |
| 25298721 | −1 | tccactgccacaagagtccc | tgg |
| 25298725 | 1 | ctatataccagggactcttg | tgg |
| 25298731 | 1 | accagggactcttgtggcag | tgg |
| 25298749 | 1 | agtggaaaatacaactctca | tgg |
| 25298767 | 1 | catggaacgtctgttccaga | agg |
| 25298771 | −1 | ttattggcagtctttccttc | tgg |
| 25298787 | −1 | ttgcctattttattgtttat | tgg |
| 25298795 | 1 | ctgccaataaacaataaaat | agg |
| 25298822 | 1 | agatatagcatgttagagag | tgg |
| 25298840 | −1 | ctccatttcatttttatctg | tgg |
| 25298849 | 1 | taccacagataaaaatgaaa | tgs |
| 25298872 | 1 | agaaaagaaacacgaaaagt | tgg |
| 25298873 | 1 | gaaaagaaacacgaaaagtt | ggg |
| 25298874 | 1 | aaaagaaacacgaaaagttg | ggg |
| 25298881 | 1 | acacgaaaagttggggagag | agg |
| 25298897 | 1 | agagaggataactgtttgag | agg |
| 25298898 | 1 | gagaggataactgtttgaga | ggg |
| 25298901 | 1 | aggataactgtttgagaggg | tgg |
| 25298906 | 1 | aactgtttgagagggggcc | agg |
| 25298907 | 1 | actgtttgagagggtggcca | ggg |
| 25298908 | 1 | ctgtttgagagggtggccag | ggg |
| 25298913 | −1 | tgataagatgaagctgcccc | tgg |
| 25298929 | 1 | ggcagcttcatcttatcaag | agg |
| 25298930 | 1 | gcagcttcatcttatcaaga | 555 |
| 25298956 | 1 | ttttttgagtacagacctga | agg |
| 25298960 | −1 | cttgtgcactcgttaccttc | agg |
| 25298979 | 1 | taacgagtgcacaagccata | tgg |
| 25298980 | 1 | aacgagtgcacaagccatat | ggg |
| 25298983 | −1 | gctgttctcaggtacccata | tgg |
| 25298994 | −1 | ATTGTTCTGCcgctgttctc | agg |
| 25298996 | 1 | atatgggtacctgagaacag | cgG |
| 25299007 | 1 | tgagaacagcgGCAGAACAA | TGG |
| 25299011 | 1 | aacagcgGCAGAACAATGGC | AGG |
| 25299012 | 1 | acagcgGCAGAACAATGGCA | GGG |
| 25299018 | 1 | GCAGAACAATGGCAGGGTGC | Tgg |
| 25299019 | 1 | CAGAACAATGGCAGGGTGCT | ggg |
| 25299022 | 1 | AACAATGGCAGGGTGCTggg | agg |
| 25299023 | 1 | ACAATGGCAGGGTGCTggga | ggg |
| 25299042 | −1 | acaattctaaacagcgtggc | tgg |
| 25299046 | −1 | gctgacaattctaaacagcg | tgs |
| 25299063 | 1 | tgtttagaattgtcagcaca | tgg |
| 25299100 | 1 | aaaaaaaaaaaaaaacaggc | tgg |
| 25299101 | 1 | aaaaaaaaaaaaaacaggct | ggg |
| 25299109 | 1 | aaaaaacaggctgggagcag | tgg |
| 25299127 | −1 | tcccaaagcgctgggattac | agg |
| 25299135 | −1 | ccttggcctcccaaagcgct | ggg |
| 25299136 | 1 | tgcctgtaatcccagcgctt | tgg |
| 25299136 | −1 | gccttggcctcccaaagcgc | tgg |
| 25299137 | 1 | gcctgtaatcccagcgcttt | ggg |
| 25299140 | 1 | tgtaatcccagcgctttggg | agg |
| 25299146 | 1 | cccagcgctttgggaggcca | agg |
| 25299149 | 1 | agcgctttgggaggccaagg | cgg |
| 25299152 | −1 | ctcaagtgatccatccgcct | tgg |
| 25299153 | 1 | ctttgggaggccaaggcgga | tgg |
| 25299164 | 1 | caaggcggatggatcacttg | agg |
| 25299169 | 1 | cggatggatcacttgaggtc | agg |
| 25299183 | 1 | gaggtcaggagttcgagacc | agg |
| 25299187 | 1 | tcaggagttcgagaccaggc | tgg |
| 25299188 | 1 | caggagttcgagaccaggct | ggg |
| 25299189 | 1 | aggagttcgagaccaggctg | ggg |
| 25299190 | −1 | tttcaccatgttccccagcc | tgg |
| 25299196 | 1 | cgagaccaggctggggaaca | tgg |
| 25299213 | −1 | ttgtatttttagtagagacg | ggg |
| 25299214 | −1 | tttgtatttttagtagagac | ggg |
| 25299215 | −1 | ttttgtatttttagtagaga | cgg |
| 25299235 | 1 | ctaaaaatacaaaaattagc | cgg |
| 25299236 | 1 | taaaaatacaaaaattagcc | ggg |
| 25299241 | 1 | atacaaaaattagccgggca | cgg |
| 25299243 | −1 | caggcacccaccaccgtgcc | cgg |
| 25299244 | 1 | caaaaattagccgggcacgg | tgg |
| 25299247 | 1 | aaattagccgggcacggtgg | tgg |
| 25299248 | 1 | aattagccgggcacggtggt | ggg |
| 25299262 | −1 | tcccaagtagctgggattac | agg |
| 25299270 | −1 | cttcagcctcccaagtagct | ggg |
| 25299271 | 1 | tgcctgtaatcccagctact | tgg |
| 25299271 | −1 | gcttcagcctcccaagtagc | tgg |
| 25299272 | 1 | gcctgtaatcccagctactt | ggg |
| 25299275 | 1 | tgtaatcccagctacttggg | agg |
| 25299285 | 1 | gctacttgggaggctgaagc | agg |
| 25299306 | 1 | ggagaatcgcttgaacccaa | cgg |
| 25299307 | 1 | gagaatcgcttgaacccaac | ggg |
| 25299310 | 1 | aatcgcttgaacccaacggg | tgg |
| 25299310 | −1 | cactgcaacctccacccgtt | ggg |
| 25299311 | −1 | tcactgcaacctccacccgt | tgg |
| 25299313 | 1 | cgcttgaacccaacggggg | agg |
| 25299332 | 1 | gaggttgcagtgagccaaga | tgg |
| 25299335 | −1 | agagtgcactggtgccatct | tgg |
| 25299346 | −1 | gtcgccaggctagagtgcac | tgg |
| 25299353 | 1 | ggcaccagtgcactctagcc | tgg |
| 25299360 | −1 | cggagtctcactctgtcgcc | agg |
| 25299380 | −1 | ttatttatttatttttgaga | cgg |
| 25299429 | 1 | aagcagacagactttttagt | tgg |
| 25299459 | −1 | cggggtgccttgtctgtaga | ggg |
| 25299460 | −1 | tcggggtgccttgtctgtag | agg |
| 25299463 | 1 | ttagacaccctctacagaca | agg |
| 25299477 | −1 | accctgggtgcaagcaatcg | ggg |
| 25299478 | −1 | caccctgggtgcaagcaatc | ggg |
| 25299479 | −1 | ccaccctgggtgcaagcaat | cgg |
| 25299486 | 1 | caccccgattgcttgcaccc | agg |
| 25299487 | 1 | accccgattgcttgcaccca | ggg |
| 25299490 | 1 | ccgattgcttgcacccaggg | tgg |
| 25299492 | −1 | tggagggagtagtccaccct | ggg |
| 25299493 | −1 | gtggagggagtagtccaccc | tgg |
| 25299508 | −1 | tgtaacaagggcagggtgga | ggg |
| 25299509 | −1 | gtgtaacaagggcagggtgg | agg |
| 25299512 | −1 | AGggtgtaacaagggcaggg | tgg |
| 25299515 | −1 | GCCAGggtgtaacaagggca | ggg |
| 25299516 | −1 | AGCCAGggtgtaacaagggc | agg |
| 25299520 | −1 | CCCCAGCCAGggtgtaacaa | ggg |
| 25299521 | −1 | CCCCCAGCCAGggtgtaaca | agg |
| 25299525 | 1 | accctgcccttgttacaccC | TGG |
| 25299529 | 1 | tgcccttgttacaccCTGGC | TGG |
| 25299530 | 1 | gcccttgttacaccCTGGCT | GGG |
| 25299531 | 1 | cccttgttacaccCTGGCTG | GGG |
| 25299531 | −1 | GAAATGCTGACCCCCAGCCA | Ggg |
| 25299532 | 1 | ccttgttacaccCTGGCTGG | GGG |
| 25299532 | −1 | TGAAATGCTGACCCCCAGCC | AGg |
| 25299545 | 1 | TGGCTGGGGGTCAGCATTTC | AGG |
| 25299566 | 1 | GGCAGCTGAATGACCCAAAG | TGG |
| 25299567 | 1 | GCAGCTGAATGACCCAAAGT | GGG |
| 25299568 | −1 | cactagcGTGTTCCCACTTT | GGG |
| 25299569 | −1 | ccactagcGTGTTCCCACTT | TGG |
| 25299580 | 1 | CCAAAGTGGGAACACgctag | tgg |
| 25299581 | 1 | CAAAGTGGGAACACgctagt | ggg |
| 25299588 | 1 | GGAACACgctagtgggtttg | agg |
| 25299600 | 1 | tgggtttgaggatgagcaag | tgg |
| 25299603 | 1 | gtttgaggatgagcaagtgg | agg |
| 25299606 | 1 | tgaggatgagcaagtggagg | agg |
| 25299607 | 1 | gaggatgagcaagtggagga | ggg |
| 25299614 | 1 | agcaagtggaggagggcaat | agg |
| 25299617 | 1 | aagtggaggagggcaatagg | agg |
| 25299631 | 1 | aataggaggtgacgcccgag | agg |
| 25299634 | −1 | ccactctcacctgacctctc | ggg |
| 25299635 | −1 | tccactctcacctgacctct | cgg |
| 25299636 | 1 | gaggtgacgcccgagaggtc | agg |
| 25299645 | 1 | cccgagaggtcaggtgagag | tgg |
| 25299655 | 1 | caggtgagagtggatcctgc | agg |
| 25299656 | 1 | aggtgagagtggatcctgca | ggg |
| 25299659 | −1 | ggttcttgccacgaccctgc | agg |
| 25299662 | 1 | gagtggatcctgcagggtcg | tgg |
| 25299673 | 1 | gcagggtcgtggcaagaacc | tgg |
| 25299680 | −1 | gtcactcaaagtcaaggtcc | agg |
| 25299686 | −1 | tcccatgtcactcaaagtca | agg |
| 25299695 | 1 | gaccttgactttgagtgaca | tgg |
| 25299696 | 1 | accttgactttgagtgacat | ggg |
| 25299705 | 1 | ttgagtgacatgggagccgc | tgg |
| 25299708 | 1 | agtgacatgggagccgctgg | agg |
| 25299710 | −1 | ctctgctcagaagcctccag | cgg |
| 25299722 | 1 | cgctggaggcttctgagcag | agg |
| 25299794 | 1 | tgtcactctgtcgctgaagc | tgg |
| 25299804 | 1 | tcgctgaagctggagtgcag | tgg |
| 25299837 | −1 | cactggaacctgggaggcgg | agg |
| 25299840 | 1 | cactatagcctccgcctccc | agg |
| 25299840 | −1 | attcactggaacctgggagg | cgg |
| 25299843 | −1 | gagattcactggaacctggg | agg |
| 25299846 | −1 | caggagattcactggaacct | ggg |
| 25299847 | −1 | gcaggagattcactggaacc | tgg |
| 25299854 | −1 | ggctgatgcaggagattcac | tgg |
| 25299865 | −1 | tctacctgggaggctgatgc | agg |
| 25299872 | 1 | atctcctgcatcagcctccc | agg |
| 25299875 | −1 | tgtaatcctatctacctggg | agg |
| 25299878 | −1 | gcttgtaatcctatctacct | ggg |
| 25299879 | −1 | tgcttgtaatcctatctacc | tgg |
| 25299880 | 1 | catcagcctcccaggtagat | agg |
| 25299907 | 1 | caagcaagcatcaccacgcc | tgg |
| 25299909 | −1 | atacaaaaattagccaggcg | tgg |
| 25299914 | −1 | taaaaatacaaaaattagcc | agg |
| 25299936 | 1 | tttgtatttttagtagagac | agg |
| 25299937 | 1 | ttgtatttttagtagagaca | ggg |
| 25299951 | 1 | gagacagggttttgccatgt | tgg |
| 25299954 | −1 | cgataccagcctggccaaca | tgg |
| 25299956 | 1 | agggttttgccatgttggcc | agg |
| 25299960 | 1 | ttttgccatgttggccaggc | tgg |
| 25299963 | −1 | tcaggagttcgataccagcc | tgg |
| 25299981 | 1 | ggtatcgaactcctgacctc | agg |
| 25299981 | −1 | tgggtggatcacctgaggtc | agg |
| 25299986 | −1 | tgaggtgggtggatcacctg | agg |
| 25299997 | −1 | ctttgggaggctgaggtggg | tgg |
| 25300000 | −1 | gcactttgggaggctgaggt | ggg |
| 25300001 | −1 | agcactttgggaggctgagg | tgg |
| 25300004 | −1 | cccagcactttgggaggctg | agg |
| 25300010 | −1 | tgtaatcccagcactttggg | agg |
| 25300013 | −1 | gcctgtaatcccagcacttt | ggg |
| 25300014 | 1 | acctcagcctoccaaagtgc | tgg |
| 25300014 | −1 | tgcctgtaatcccagcactt | tgg |
| 25300015 | 1 | cctcagcctcccaaagtgct | ggg |
| 25300023 | 1 | tcccaaagtgctgggattac | agg |
| 25300060 | −1 | aataccaaactaaggtcttc | agg |
| 25300067 | 1 | atttcctgaagaccttagtt | tgg |
| 25300068 | −1 | cttcttataataccaaacta | agg |
| 25300084 | 1 | gtttggtattataagaagtc | tgg |
| 25300132 | −1 | cagcggaattttaactctgc | ggg |
| 25300133 | −1 | tcagcggaattttaactctg | cgg |
| 25300149 | −1 | cactgattcctacttctcag | cgg |
| 25300152 | 1 | ttaaaattccgctgagaagt | agg |
| 25300163 | 1 | ctgagaagtaggaatcagtg | agg |
| 25300178 | 1 | cagtgaggtgcgtgtccatg | tgg |
| 25300179 | 1 | agtgaggtgcgtgtccatgt | ggg |
| 25300182 | −1 | aggtgtggcaaaaacccaca | tgg |
| 25300197 | −1 | gaccaaggttcacttaggtg | tgg |
| 25300202 | −1 | cttttgaccaaggttcactt | agg |
| 25300206 | 1 | tgccacacctaagtgaacct | tgg |
| 25300212 | −1 | ctcttatatgcttttgacca | agg |
| 25300234 | 1 | agcatataagagctactgAT | Agg |
| 25300238 | 1 | tataagagctactgATAggc | cgg |
| 25300239 | 1 | ataagagctactgATAggcc | ggg |
| 25300244 | 1 | agctactgATAggccgggtg | tgg |
| 25300246 | −1 | caggcatgagccaccacacc | cgg |
| 25300247 | 1 | tactgATAggccgggtgtgg | tgg |
| 25300265 | −1 | tcccaaagtgctgagattac | agg |
| 25300274 | 1 | tgcctgtaatctcagcactt | tgg |
| 25300275 | 1 | gcctgtaatctcagcacttt | |
| 25300278 | 1 | tgtaatctcagcactttggg | agg |
| 25300279 | 1 | gtaatctcagcactttggga | ggg |
| 25300283 | 1 | tctcagcactttgggaggga | agg |
| 25300299 | 1 | gggaaggatctcttgagccc | agg |
| 25300305 | −1 | caggctggtcttgaactcct | ggg |
| 25300306 | −1 | tcaggctggtcttgaactcc | tgg |
| 25300320 | −1 | tcttgctatgttgctcaggc | tgg |
| 25300324 | −1 | ggaatcttgctatgttgctc | agg |
| 25300345 | −1 | ttttaaattttgtgtaaaga | tgg |
| 25300361 | 1 | tttacacaaaatttaaaaat | tgg |
| 25300366 | 1 | acaaaatttaaaaattggcc | agg |
| 25300371 | 1 | atttaaaaattggccaggca | tgg |
| 25300373 | −1 | caggaatgtacaaccatgcc | tgg |
| 25300392 | −1 | tcctgagtagctgggattac | agg |
| 25300400 | −1 | cctcagcctcctgagtagct | ggg |
| 25300401 | −1 | acctcagcctcctgagtagc | tgg |
| 25300402 | 1 | tcctgtaatcccagctactc | agg |
| 25300405 | 1 | tgtaatcccagctactcagg | agg |
| 25300411 | 1 | cccagctactcaggaggctg | agg |
| 25300414 | 1 | agctactcaggaggctgagg | tgg |
| 25300415 | 1 | gctactcaggaggctgaggt | ggg |
| 25300418 | 1 | actcaggaggctgaggtggg | agg |
| 25300433 | 1 | gtgggaggattgcttgagcc | tgg |
| 25300434 | 1 | tgggaggattgcttgagcct | ggg |
| 25300440 | 1 | gattgcttgagcctgggagt | tgg |
| 25300440 | −1 | cactgtagtctccaactccc | agg |
| 25300459 | 1 | ttggagactacagtgagctg | tgg |
| 25300471 | −1 | caagctggagtgcagtggtg | tgg |
| 25300476 | −1 | ttgctcaagctggagtgcag | tgg |
| 25300486 | −1 | tcttgctccattgctcaagc | tgg |
| 25300490 | 1 | ctgcactccagcttgagcaa | tgg |
| 25300524 | 1 | gtctcaaaaaaaaaaaaaaa | agg |
| 25300529 | 1 | aaaaaaaaaaaaaaaaggcc | agg |
| 25300536 | −1 | caggcatgagccactgcgcc | tgg |
| 25300537 | 1 | aaaaaaaaggccaggcgcag | tgg |
| 25300555 | −1 | tcccaaagtgctgggattac | agg |
| 25300563 | −1 | cctcggcctcccaaagtgct | ggg |
| 25300564 | 1 | tgcctgtaatcccagcactt | tgg |
| 25300564 | −1 | gcctcggcctcccaaagtgc | tgg |
| 25300565 | 1 | gcctgtaatcccagcacttt | ggg |
| 25300568 | 1 | tgtaatcccagcactttggg | agg |
| 25300574 | 1 | cccagcactttgggaggccg | agg |
| 25300577 | 1 | agcactttgggaggccgagg | cgg |
| 25300578 | 1 | gcactttgggaggccgaggc | ggg |
| 25300580 | −1 | ctcaggcgatccacccgcct | cgg |
| 25300581 | 1 | ctttgggaggccgaggcggg | tgg |
| 25300592 | 1 | cgaggcgggtggatcgcctg | agg |
| 25300597 | 1 | cgggtggatcgcctgaggtc | agg |
| 25300597 | −1 | ggtctcaaactcctgacctc | agg |
| 25300615 | 1 | tcaggagtttgagaccagcc | tgg |
| 25300618 | −1 | tttcaccgtgtttgccaggc | tgg |
| 25300622 | −1 | ggggtttcaccgtgtttgcc | agg |
| 25300624 | 1 | tgagaccagcctggcaaaca | cgg |
| 25300641 | −1 | ttgtatttttagtagagatg | ggg |
| 25300642 | −1 | tttgtatttttagtagagat | ggg |
| 25300643 | −1 | ttttgtatttttagtagaga | tgg |
| 25300670 | −1 | caggcatgcgccactacgct | ggg |
| 25300671 | 1 | acaaaattagcccagcgtag | tgg |
| 25300671 | −1 | acaggcatgcgccactacgc | tgg |
| 25300689 | −1 | tccctagtagctgggattac | agg |
| 25300697 | −1 | cctcagcttccctagtagct | ggg |
| 25300698 | 1 | tgcctgtaatcccagctact | agg |
| 25300698 | −1 | gcctcagcttccctagtagc | tgg |
| 25300699 | 1 | gcctgtaatcccagctacta | ggg |
| 25300708 | 1 | cccagctactagggaagctg | agg |
| 25300712 | 1 | gctactagggaagctgaggc | agg |
| 25300730 | 1 | gcaggagaatcgcgtgaacc | tgg |
| 25300731 | 1 | caggagaatcgcgtgaacct | ggg |
| 25300734 | 1 | gagaatcgcgtgaacctggg | agg |
| 25300737 | −1 | cactggaacatttgcctccc | agg |
| 25300754 | −1 | atggcacgatctcggctcac | tgg |
| 25300762 | −1 | ggagtgcaatggcacgatct | cgg |
| 25300773 | −1 | ctgcccaggctggagtgcaa | tgg |
| 25300780 | 1 | cgtgccattgcactccagcc | tgg |
| 25300781 | 1 | gtgccattgcactccagcct | ggg |
| 25300783 | −1 | CCAGCAGgctctgcccaggc | tgg |
| 25300787 | −1 | CAACCCAGCAGgctctgccc | agg |
| 25300794 | 1 | ccagcctgggcagagcCTGC | TGG |
| 25300795 | 1 | cagcctgggcagagcCTGCT | GGG |
| 25300798 | −1 | CTTACCCAGCCCAACCCAGC | AGg |
| 25300799 | 1 | ctgggcagagcCTGCTGGGT | TGG |
| 25300800 | 1 | tgggcagagcCTGCTGGGTT | GGG |
| 25300804 | 1 | cagagcCTGCTGGGTTGGGC | TGG |
| 25300805 | 1 | agagcCTGCTGGGTTGGGCT | GGG |
| 25300830 | 1 | AGCTCTGAACACCAGTCTCA | TGG |
| 25300830 | −1 | GTGACTTGAAGCCATGAGAC | TGG |
| 25300854 | −1 | AGTTCAGAGCTTCACTTAGG | AGG |
| 25300857 | −1 | GAAAGTTCAGAGCTTCACTT | AGG |
| 25300875 | 1 | GAAGCTCTGAACTTTCTCCA | AGG |
| 25300881 | −1 | GGGCAAGCCCTGATAGTCCT | TGG |
| 25300884 | 1 | AACTTTCTCCAAGGACTATC | AGG |
| 25300885 | 1 | ACTTTCTCCAAGGACTATCA | GGG |
| 25300895 | 1 | AGGACTATCAGGGCTTGCCC | CGG |
| 25300896 | 1 | GGACTATCAGGGCTTGCCCC | GGG |
| 25300901 | −1 | GTGTCGGCATCCTCTGCCCG | GGG |
| 25300902 | 1 | TCAGGGCTTGCCCCGGGCAG | AGG |
| 25300902 | −1 | AGTGTCGGCATCCTCTGCCC | GGG |
| 25300903 | −1 | GAGTGTCGGCATCCTCTGCC | CGG |
| 25300917 | −1 | CCAGTAAGAGCAGTGAGTGT | CGG |
| 25300928 | 1 | CCGACACTCACTGCTCTTAC | TGG |
| 25300929 | 1 | CGACACTCACTGCTCTTACT | GGG |
| 25300960 | −1 | AGATGTGCATCATGTTCATG | TGG |
| 25300993 | 1 | TACGTGTTCGCAGCCTATTT | TGG |
| 25300994 | 1 | ACGTGTTCGCAGCCTATTTT | GGG |
| 25300995 | −1 | GGCCACAGACAGCCCAAAAT | AGG |
| 25301004 | 1 | AGCCTATTTTGGGCTGTCTG | TGG |
| 25301009 | 1 | ATTTTGGGCTGTCTGTGGCC | TGG |
| 25301016 | −1 | TAGAGGCTTTGGCAGGCACC | AGG |
| 25301023 | −1 | CCTCGGGTAGAGGCTTTGGC | AGG |
| 25301027 | −1 | GTTCCCTCGGGTAGAGGCTT | TGG |
| 25301033 | −1 | TCCTCCGTTCCCTCGGGTAG | AGG |
| 25301034 | 1 | CCTGCCAAAGCCTCTACCCG | AGG |
| 25301035 | 1 | CTGCCAAAGCCTCTACCCGA | GGG |
| 25301039 | −1 | TCTTTATCCTCCGTTCCCTC | GGG |
| 25301040 | 1 | AAAGCCTCTACCCGAGGGAA | CGG |
| 25301040 | −1 | ATCTTTATCCTCCGTTCCCT | CGG |
| 25301043 | 1 | GCCTCTACCCGAGGGAACGG | AGG |
| 25301078 | −1 | CCCAGCATGGCAGACAAACT | GGG |
| 25301079 | −1 | ACCCAGCATGGCAGACAAAC | TGG |
| 25301088 | 1 | ACCCAGTTTGTCTGCCATGC | TGG |
| 25301089 | 1 | CCCAGTTTGTCTGCCATGCT | GGG |
| 25301091 | −1 | cacctTGTCCTTACCCAGCA | TGG |
| 25301094 | 1 | TTTGTCTGCCATGCTGGGTA | AGG |
| 25301100 | 1 | TGCCATGCTGGGTAAGGACA | agg |
| 25301103 | 1 | CATGCTGGGTAAGGACAagg | tgg |
| 25301104 | 1 | ATGCTGGGTAAGGACAaggt | ggg |
| 25301105 | 1 | TGCTGGGTAAGGACAaggtg | ggg |
| 25301112 | 1 | TAAGGACAaggtggggtgag | tgg |
| 25301124 | 1 | ggggtgagtggtctcctact | tgg |
| 25301125 | 1 | gggtgagtggtctcctactt | ggg |
| 25301127 | −1 | ccattctgctcagcccaagt | agg |
| 25301138 | 1 | cctacttgggctgagcagaa | tgg |
| 25301149 | 1 | tgagcagaatggctcagaaa | agg |
| 25301155 | 1 | gaatggctcagaaaaggctc | tgg |
| 25301179 | −1 | caggggaacttggtaaagga | ggg |
| 25301180 | −1 | ccaggggaacttggtaaagg | agg |
| 25301183 | −1 | cacccaggggaacttggtaa | agg |
| 25301189 | −1 | ttcagacacccaggggaact | tgg |
| 25301191 | 1 | cctcctttaccaagttcccc | tgg |
| 25301192 | 1 | ctcctttaccaagttcccct | ggg |
| 25301196 | −1 | gaagggcttcagacacccag | ggg |
| 25301197 | −1 | ggaagggcttcagacaccca | ggg |
| 25301198 | −1 | tggaagggcttcagacaccc | agg |
| 25301213 | −1 | agaaatgaatcatgatggaa | ggg |
| 25301214 | −1 | aagaaatgaatcatgatgga | agg |
| 25301218 | −1 | ctcaaagaaatgaatcatga | tgg |
| 25301261 | −1 | CTGTGAAGTGCTTAATTCAA | AGG |
| 25301278 | 1 | AATTAAGCACTTCACAGAGC | AGG |
| 25301284 | 1 | GCACTTCACAGAGCAGGTTC | AGG |
| 25301287 | 1 | CTTCACAGAGCAGGTTCAGG | Agg |
| 25301292 | 1 | CAGAGCAGGTTCAGGAggcc | tgg |
| 25301293 | 1 | AGAGCAGGTTCAGGAggcct | ggg |
| 25301294 | 1 | GAGCAGGTTCAGGAggcctg | ggg |
| 25301299 | −1 | ggttgaaatctgcatacccc | agg |
| 25301317 | 1 | tatgcagatttcaaccctct | tgg |
| 25301320 | −1 | caaggaaacaaaggccaaga | ggg |
| 25301321 | −1 | acaaggaaacaaaggccaag | agg |
| 25301329 | −1 | ttttacagacaaggaaacaa | agg |
| 25301338 | −1 | CTAAccacattttacagaca | agg |
| 25301345 | 1 | gtttccttgtctgtaaaatg | tgg |
| 25301353 | 1 | gtctgtaaaatgtggTTAGC | TGG |
| 25301372 | 1 | CTGGTATCAGCTTGAGAGCT | CGG |
| 25301375 | 1 | GTATCAGCTTGAGAGCTCGG | AGG |
| 25301376 | 1 | TATCAGCTTGAGAGCTCGGA | GGG |
| 25301377 | 1 | ATCAGCTTGAGAGCTCGGAG | GGG |
| 25301400 | −1 | TTGTCACTTAGAGTTAGATG | GGG |
| 25301401 | −1 | CTTGTCACTTAGAGTTAGAT | GGG |
| 25301402 | −1 | CCTTGTCACTTAGAGTTAGA | TGG |
| 25301413 | 1 | CCATCTAACTCTAAGTGACA | AGG |
| 25301434 | 1 | GGCTGAGACTCTCCAGCCCT | AGG |
| 25301435 | −1 | TTGGATGAGAATCCTAGGGC | TGG |
| 25301439 | −1 | GGTTTTGGATGAGAATCCTA | GGG |
| 25301440 | −1 | GGGTTTTGGATGAGAATCCT | AGG |
| 25301454 | −1 | GTCTGAGCCTCGAGGGGTTT | TGG |
| 25301458 | 1 | TTCTCATCCAAAACCCCTCG | AGG |
| 25301460 | −1 | CCAAAGGTCTGAGCCTCGAG | GGG |
| 25301461 | −1 | TCCAAAGGTCTGAGCCTCGA | GGG |
| 25301462 | −1 | CTCCAAAGGTCTGAGCCTCG | AGG |
| 25301471 | 1 | CCCCTCGAGGCTCAGACCTT | TGG |
| 25301476 | −1 | GAATCACACTCCTGCTCCAA | AGG |
| 25301477 | 1 | GAGGCTCAGACCTTTGGAGC | AGG |
| 25301490 | 1 | TTGGAGCAGGAGTGTGATTC | TGG |
| 25301502 | −1 | TGGGGGCCAGAGAGGGTGGT | TGG |
| 25301506 | −1 | CGCCTGGGGGCCAGAGAGGG | TGG |
| 25301507 | 1 | TTCTGGCCAACCACCCTCTC | TGG |
| 25301509 | −1 | GGGCGCCTGGGGGCCAGAGA | GGG |
| 25301510 | −1 | AGGGCGCCTGGGGGCCAGAG | AGG |
| 25301515 | 1 | AACCACCCTCTCTGGCCCCC | AGG |
| 25301519 | −1 | CACAAGAAGAGGGCGCCTGG | GGG |
| 25301520 | −1 | CCACAAGAAGAGGGCGCCTG | GGG |
| 25301521 | −1 | TCCACAAGAAGAGGGCGCCT | GGG |
| 25301522 | −1 | ATCCACAAGAAGAGGGCGCC | TGG |
| 25301529 | −1 | CCAGAACATCCACAAGAAGA | GGG |
| 25301530 | −1 | GCCAGAACATCCACAAGAAG | AGG |
| 25301531 | 1 | CCCCAGGCGCCCTCTTCTTG | TGG |
| 25301540 | 1 | CCCTCTTCTTGTGGATGTTC | TGG |
| 25301552 | −1 | AGCAGAGCAGAGTTGAAACT | TGG |
| 25301582 | 1 | TGCTGAGAAGTCCAATCGAA | AGG |
| 25301582 | −1 | ACGGCATTCTTCCTTTCGAT | TGG |
| 25301601 | −1 | AGCATAGTAGGTGTTGAACA | CGG |
| 25301613 | −1 | GCTGACTGCTACAGCATAGT | AGG |
| 25301628 | 1 | CTATGCTGTAGCAGTCAGCG | TGG |
| 25301644 | 1 | AGCGTGGTGACAGCCATCTC | AGG |
| 25301645 | 1 | GCGTGGTGACAGCCATCTCA | GGG |
| 25301646 | −1 | AGCCAAGGATGACCCTGAGA | TGG |
| 25301655 | 1 | AGCCATCTCAGGGTCATCCT | TGG |
| 25301661 | −1 | CTTCCCTTGGGGGTGAGCCA | AGG |
| 25301668 | 1 | TCATCCTTGGCTCACCCCCA | AGG |
| 25301669 | 1 | CATCCTTGGCTCACCCCCAA | GGG |
| 25301671 | −1 | CCTTGCTGATCTTCCCTTGG | GGG |
| 25301672 | −1 | ACCTTGCTGATCTTCCCTTG | GGG |
| 25301673 | −1 | CACCTTGCTGATCTTCCCTT | GGG |
| 25301674 | −1 | TCACCTTGCTGATCTTCCCT | TGG |
| 25301682 | 1 | CCCCCAAGGGAAGATCAGCA | AGG |
| 25301690 | 1 | GGAAGATCAGCAAGGTGAGC | AGG |
| 25301691 | 1 | GAAGATCAGCAAGGTGAGCA | GGG |
| 25301703 | 1 | GGTGAGCAGGGCGCTGCCCT | TGG |
| 25301704 | 1 | GTGAGCAGGGCGCTGCCCTT | GGG |
| 25301708 | −1 | TAGACCCAAGTGCTGCCCAA | GGG |
| 25301709 | −1 | TTAGACCCAAGTGCTGCCCA | AGG |
| 25301714 | 1 | CGCTGCCCTTGGGCAGCACT | TGG |
| 25301715 | 1 | GCTGCCCTTGGGCAGCACTT | GGG |
| 25301724 | 1 | GGGCAGCACTTGGGTCTAAC | AGG |
| 25301755 | −1 | GCTGGCCCTGGGGTGGGGAG | GGG |
| 25301756 | −1 | CGCTGGCCCTGGGGTGGGGA | GGG |
| 25301757 | −1 | ACGCTGGCCCTGGGGTGGGG | AGG |
| 25301760 | 1 | TTTATGCCCCTCCCCACCCC | AGG |
| 25301760 | −1 | CCCACGCTGGCCCTGGGGTG | GGG |
| 25301761 | 1 | TTATGCCCCTCCCCACCCCA | GGG |
| 25301761 | −1 | ACCCACGCTGGCCCTGGGGT | GGG |
| 25301762 | −1 | AACCCACGCTGGCCCTGGGG | TGG |
| 25301765 | −1 | CCCAACCCACGCTGGCCCTG | GGG |
| 25301766 | −1 | TCCCAACCCACGCTGGCCCT | GGG |
| 25301767 | −1 | CTCCCAACCCACGCTGGCCC | TGG |
| 25301770 | 1 | TCCCCACCCCAGGGCCAGCG | TGG |
| 25301771 | 1 | CCCCACCCCAGGGCCAGCGT | GGG |
| 25301773 | −1 | TGCCCTCTCCCAACCCACGC | TGG |
| 25301775 | 1 | ACCCCAGGGCCAGCGTGGGT | TGG |
| 25301776 | 1 | CCCCAGGGCCAGCGTGGGTT | GGG |
| 25301781 | 1 | GGGCCAGCGTGGGTTGGGAG | AGG |
| 25301782 | 1 | GGCCAGCGTGGGTTGGGAGA | GGG |
| 25301790 | 1 | TGGGTTGGGAGAGGGCATGC | CGG |
| 25301791 | 1 | GGGTTGGGAGAGGGCATGCC | GGG |
| 25301794 | 1 | TTGGGAGAGGGCATGCCGGG | TGG |
| 25301797 | 1 | GGAGAGGGCATGCCGGGTGG | TGG |
| 25301798 | −1 | GCAGGCACAGCTCCACCACC | CGG |
| 25301816 | −1 | TAGAGCTCCACTGTAGAGGC | AGG |
| 25301820 | 1 | AGCTGTGCCTGCCTCTACAG | TGG |
| 25301820 | −1 | TACCTAGAGCTCCACTGTAG | AGG |
| 25301829 | 1 | TGCCTCTACAGTGGAGCTCT | AGG |
| 25301840 | 1 | TGGAGCTCTAGGTAGAATGC | TGG |
| 25301841 | 1 | GGAGCTCTAGGTAGAATGCT | GGG |
| 25301844 | 1 | GCTCTAGGTAGAATGCTGGG | TGG |
| 25301853 | 1 | AGAATGCTGGGTGGTCACAG | TGG |
| 25301854 | 1 | GAATGCTGGGTGGTCACAGT | GGG |
| 25301859 | 1 | CTGGGTGGTCACAGTGGGCC | TGG |
| 25301860 | 1 | TGGGTGGTCACAGTGGGCCT | GGG |
| 25301866 | −1 | TGGACAGTCTCCTGAGTCCC | AGG |
| 25301867 | 1 | TCACAGTGGGCCTGGGACTC | AGG |
| 25301886 | −1 | CCCAGAAAGCCTTTGATCAC | TGG |
| 25301888 | 1 | GGAGACTGTCCAGTGATCAA | AGG |
| 25301896 | 1 | TCCAGTGATCAAAGGCTTTC | TGG |
| 25301897 | 1 | CCAGTGATCAAAGGCTTTCT | GGG |
| 25301898 | 1 | CAGTGATCAAAGGCTTTCTG | GGG |
| 25301899 | 1 | AGTGATCAAAGGCTTTCTGG | GGG |
| 25301923 | −1 | CTGTTTCATGTTAGCATGGA | TGG |
| 25301927 | −1 | AGGTCTGTTTCATGTTAGCA | TGG |
| 25301947 | −1 | CAGAAATGGGGTTCAAACTG | AGG |
| 25301959 | −1 | TTTAGCAACTAGCAGAAATG | GGG |
| 25301960 | −1 | CTTTAGCAACTAGCAGAAAT | GGG |
| 25301961 | −1 | ACTTTAGCAACTAGCAGAAA | TGG |
| 25301990 | −1 | TTGCTGCTGACTCTCGCTCA | TGG |
| 25302032 | −1 | GTTGGGGGGAAGAGAGAGGC | TGG |
| 25302036 | −1 | ATTTGTTGGGGGGAAGAGAG | AGG |
| 25302046 | −1 | CATTCTTGAAATTTGTTGGG | GGG |
| 25302047 | −1 | CCATTCTTGAAATTTGTTGG | GGG |
| 25302048 | −1 | TCCATTCTTGAAATTTGTTG | GGG |
| 25302049 | −1 | TTCCATTCTTGAAATTTGTT | GGG |
| 25302050 | −1 | GTTCCATTCTTGAAATTTGT | TGG |
| 25302058 | 1 | CCCCCAACAAATTTCAAGAA | TGG |
| 25302072 | −1 | TTCTCTACTTCTGATTCTGA | TGG |
| 25302105 | 1 | AGTATGtgacactagccatg | tgg |
| 25302109 | −1 | gtggcttgaccagagccaca | tgg |
| 25302111 | 1 | tgacactagccatgtggctc | tgg |
| 25302128 | −1 | tgagactcaaaacgttgaag | tgg |
| 25302143 | 1 | ttcaacgttttgagtctcag | tgg |
| 25302155 | −1 | taattcccactttacagatg | agg |
| 25302160 | 1 | cagtggcctcatctgtaaag | tgg |
| 25302161 | 1 | agtggcctcatctgtaaagt | ggg |
| 25302174 | 1 | gtaaagtgggaattaagaga | tgg |
| 25302195 | 1 | ggtgcatgtaaagtgcttAA | CGG |
| 25302196 | 1 | gtgcatgtaaagtgcttAAC | GGG |
| 25302197 | 1 | tgcatgtaaagtgcttAACG | GGG |
| 25302206 | 1 | agtgcttAACGGGGAGTAAA | TGG |
| 25302210 | 1 | cttAACGGGGAGTAAATGGT | AGG |
| 25302249 | 1 | CTATTAGTAAAGAGAGACGA | TGG |
| 25302267 | 1 | GATGGTGTGTGTGAGTCTTG | TGG |
| 25302268 | 1 | ATGGTGTGTGTGAGTCTTGT | GGG |
| 25302277 | 1 | GTGAGTCTTGTGGGCAGAGA | TGG |
| 25302278 | 1 | TGAGTCTTGTGGGCAGAGAT | GGG |
| 25302285 | 1 | TGTGGGCAGAGATGGGTGAG | AGG |
| 25302286 | 1 | GTGGGCAGAGATGGGTGAGA | GGG |
| 25302287 | 1 | TGGGCAGAGATGGGTGAGAG | GGG |
| 25302314 | 1 | AAAACAAGTTCTCATGATGA | TGG |
| 25302315 | 1 | AAACAAGTTCTCATGATGAT | GGG |
| 25302316 | 1 | AACAAGTTCTCATGATGATG | GGG |
| 25302317 | 1 | ACAAGTTCTCATGATGATGG | GGG |
| 25302321 | 1 | GTTCTCATGATGATGGGGGA | AGG |
| 25302322 | 1 | TTCTCATGATGATGGGGGAA | GGG |
| 25302323 | 1 | TCTCATGATGATGGGGGAAG | GGG |
| 25302333 | 1 | ATGGGGGAAGGGGCTCCAGC | TGG |
| 25302336 | 1 | GGGGAAGGGGCTCCAGCTGG | TGG |
| 25302337 | −1 | TTCCCTCCGACACCACCAGC | TGG |
| 25302342 | 1 | GGGGCTCCAGCTGGTGGTGT | CGG |
| 25302345 | 1 | GCTCCAGCTGGTGGTGTCGG | AGG |
| 25302346 | 1 | CTCCAGCTGGTGGTGTCGGA | GGG |
| 25302354 | 1 | GGTGGTGTCGGAGGGAAGTC | TGG |
| 25302366 | 1 | GGGAAGTCTGGACAGACCAG | TGG |
| 25302369 | 1 | AAGTCTGGACAGACCAGTGG | TGG |
| 25302370 | 1 | AGTCTGGACAGACCAGTGGT | GGG |
| 25302371 | 1 | GTCTGGACAGACCAGTGGTG | GGG |
| 25302371 | −1 | TCCCACCCGAGCCCCACCAC | TGG |
| 25302376 | 1 | GACAGACCAGTGGTGGGGCT | CGG |
| 25302377 | 1 | ACAGACCAGTGGTGGGGCTC | GGG |
| 25302380 | 1 | GACCAGTGGTGGGGCTCGGG | TGG |
| 25302381 | 1 | ACCAGTGGTGGGGCTCGGGT | GGG |
| 25302384 | 1 | AGTGGTGGGGCTCGGGTGGG | AGG |
| 25302415 | 1 | GGGCTGGAGTGGAAAGAATG | TGG |
| 25302427 | −1 | TGCTGTGAAGCTGTCATCTG | TGG |
| 25302456 | 1 | CAGCAGAATTCAGTGCTAAG | AGG |
| 25302466 | 1 | CAGTGCTAAGAGGAAGTGAG | TGG |
| 25302478 | −1 | TTCTGTCACCATGGAACTCA | TGG |
| 25302481 | 1 | GTGAGTGGCCATGAGTTCCA | TGG |
| 25302487 | −1 | TCTTAGACTTTCTGTCACCA | TGG |
| 25302510 | 1 | AAAGTCTAAGACACCCAGCA | AGG |
| 25302512 | −1 | ACACCCACTCCTGCCTTGCT | GGG |
| 25302513 | −1 | GACACCCACTCCTGCCTTGC | TGG |
| 25302514 | 1 | TCTAAGACACCCAGCAAGGC | AGG |
| 25302519 | 1 | GACACCCAGCAAGGCAGGAG | TGG |
| 25302520 | 1 | ACACCCAGCAAGGCAGGAGT | GGG |
| 25302532 | 1 | GCAGGAGTGGGTGTCAACTC | AGG |
| 25302533 | 1 | CAGGAGTGGGTGTCAACTCA | GGG |
| 25302544 | 1 | GTCAACTCAGGGAAGCCCAG | AGG |
| 25302548 | −1 | CTCACCTAGGATTAGCCTCT | GGG |
| 25302549 | −1 | TCTCACCTAGGATTAGCCTC | TGG |
| 25302555 | 1 | GAAGCCCAGAGGCTAATCCT | AGG |
| 25302561 | −1 | GACACCCTCAGCTCTCACCT | AGG |
| 25302567 | 1 | CTAATCCTAGGTGAGAGCTG | AGG |
| 25302568 | 1 | TAATCCTAGGTGAGAGCTGA | GGG |
| 25302586 | 1 | GAGGGTGTCAGATAAGAGCA | AGG |
| 25302591 | 1 | TGTCAGATAAGAGCAAGGCA | AGG |
| 25302597 | 1 | ATAAGAGCAAGGCAAGGCTC | CGG |
| 25302603 | 1 | GCAAGGCAAGGCTCCGGTTC | TGG |
| 25302605 | −1 | GTCCTTCACTGCTCCAGAAC | CGG |
| 25302614 | 1 | CTCCGGTTCTGGAGCAGTGA | AGG |
| 25302637 | 1 | ACATAGCAGAGCTATGACCC | AGG |
| 25302643 | −1 | ATAAGCTGGGCCTTGTTCCT | GGG |
| 25302644 | 1 | AGAGCTATGACCCAGGAACA | AGG |
| 25302644 | −1 | AATAAGCTGGGCCTTGTTCC | TGG |
| 25302656 | −1 | GGGCCCAGTTTCAATAAGCT | GGG |
| 25302657 | −1 | TGGGCCCAGTTTCAATAAGC | TGG |
| 25302663 | 1 | AAGGCCCAGCTTATTGAAAC | TGG |
| 25302664 | 1 | AGGCCCAGCTTATTGAAACT | GGG |
| 25302676 | −1 | CTGTGCCACCCTGTGTGACT | GGG |
| 25302677 | −1 | CCTGTGCCACCCTGTGTGAC | TGG |
| 25302678 | 1 | GAAACTGGGCCCAGTCACAC | AGG |
| 25302679 | 1 | AAACTGGGCCCAGTCACACA | GGG |
| 25302682 | 1 | CTGGGCCCAGTCACACAGGG | TGG |
| 25302688 | 1 | CCAGTCACACAGGGTGGCAC | AGG |
| 25302702 | −1 | TATTATTATTATTGGCTACT | TGG |
| 25302710 | −1 | ATTGTTTTTATTATTATTAT | TGG |
| 25302743 | 1 | Taacaatgatttgtgtctac | tgg |
| 25302744 | 1 | aacaatgatttgtgtctact | ggg |
| 25302774 | 1 | tcatgttctatgccagacac | tgg |
| 25302775 | 1 | catgttctatgccagacact | ggg |
| 25302775 | −1 | aaagctcttagcccagtgtc | tgg |
| 25302794 | 1 | tgggctaagagctttatatg | tgg |
| 25302819 | −1 | ttcttcataaggttattgta | agg |
| 25302830 | −1 | ttggatgtaccttcttcata | agg |
| 25302832 | 1 | ttacaataaccttatgaaga | agg |
| 25302849 | −1 | ggccTAGAagaatggggttt | tgg |
| 25302855 | −1 | gcacctggccTAGAagaatg | ggg |
| 25302856 | −1 | tgcacctggccTAGAagaat | ggg |
| 25302857 | −1 | ctgcacctggccTAGAagaa | tgg |
| 25302858 | 1 | atccaaaaccccattctTCT | Agg |
| 25302863 | 1 | aaaccccattctTCTAggcc | agg |
| 25302870 | −1 | caggtgtgagccactgcacc | tgg |
| 25302871 | 1 | ttctTCTAggccaggtgcag | tgg |
| 25302889 | −1 | tcccaaaatattgggattac | agg |
| 25302897 | −1 | cctcagcctcccaaaatatt | ggg |
| 25302898 | 1 | cacctgtaatcccaatattt | tgg |
| 25302898 | −1 | gcctcagcctoccaaaatat | tgg |
| 25302899 | 1 | acctgtaatcccaatatttt | ggg |
| 25302902 | 1 | tgtaatcccaatattttggg | agg |
| 25302908 | 1 | cccaatattttgggaggctg | agg |
| 25302915 | 1 | ttttgggaggctgaggcaag | agg |
| 25302920 | 1 | ggaggctgaggcaagaggat | tgg |
| 25302926 | 1 | tgaggcaagaggattggttg | agg |
| 25302931 | 1 | caagaggattggttgaggcc | agg |
| 25302938 | −1 | ctgggctggtcttgaactcc | tgg |
| 25302950 | 1 | caggagttcaagaccagccc | agg |
| 25302952 | −1 | tcttgctatgttgcctgggc | tgg |
| 25302956 | −1 | agggtcttgctatgttgcct | ggg |
| 25302957 | −1 | cagggtcttgctatgttgcc | tgg |
| 25302975 | −1 | tgttttattttttagagaca | ggg |
| 25302976 | −1 | ttgttttattttttagagac | agg |
| 25303002 | −1 | CCCTGGGCAGCGGGAAGAAT | GGG |
| 25303003 | −1 | TCCCTGGGCAGCGGGAAGAA | TGG |
| 25303011 | −1 | GTGGTGTGTCCCTGGGCAGC | GGG |
| 25303012 | 1 | aCCCATTCTTCCCGCTGCCC | AGG |
| 25303012 | −1 | AGTGGTGTGTCCCTGGGCAG | CGG |
| 25303013 | 1 | CCCATTCTTCCCGCTGCCCA | GGG |
| 25303018 | −1 | CTCATTAGTGGTGTGTCCCT | GGG |
| 25303019 | −1 | ACTCATTAGTGGTGTGTCCC | TGG |
| 25303030 | −1 | GCACCCATCACACTCATTAG | TGG |
| 25303037 | 1 | CACACCACTAATGAGTGTGA | TGG |
| 25303038 | 1 | ACACCACTAATGAGTGTGAT | GGG |
| 25303046 | 1 | AATGAGTGTGATGGGTGCCT | AGG |
| 25303052 | −1 | GTCCAGGTGCTCAGCATCCT | AGG |
| 25303061 | 1 | TGCCTAGGATGCTGAGCACC | TGG |
| 25303068 | −1 | GGGAATGAGCTGGGAAGTCC | AGG |
| 25303077 | −1 | CAGCATTTAGGGAATGAGCT | GGG |
| 25303078 | −1 | GCAGCATTTAGGGAATGAGC | TGG |
| 25303088 | −1 | CCCTGATTGTGCAGCATTTA | GGG |
| 25303089 | −1 | ACCCTGATTGTGCAGCATTT | AGG |
| 25303098 | 1 | TCCCTAAATGCTGCACAATC | AGG |
| 25303099 | 1 | CCCTAAATGCTGCACAATCA | GGG |
| 25303119 | −1 | ACTACTGCCTCTTAGGCTCA | GGG |
| 25303120 | −1 | CACTACTGCCTCTTAGGCTC | AGG |
| 25303123 | 1 | AACTGTGCCCTGAGCCTAAG | AGG |
| 25303126 | −1 | CCAGCTCACTACTGCCTCTT | AGG |
| 25303137 | 1 | CCTAAGAGGCAGTAGTGAGC | TGG |
| 25303149 | −1 | CCTTCATCAGTGGACATGAT | GGG |
| 25303150 | −1 | TCCTTCATCAGTGGACATGA | TGG |
| 25303159 | −1 | GGCTACGTGTCCTTCATCAG | TGG |
| 25303160 | 1 | CCCATCATGTCCACTGATGA | AGG |
| 25303180 | 1 | AGGACACGTAGCCCCAACAC | AGG |
| 25303180 | −1 | ACCACTTCTCCCCTGTGTTG | GGG |
| 25303181 | 1 | GGACACGTAGCCCCAACACA | GGG |
| 25303181 | −1 | AACCACTTCTCCCCTGTGTT | GGG |
| 25303182 | 1 | GACACGTAGCCCCAACACAG | GGG |
| 25303182 | −1 | AAACCACTTCTCCCCTGTGT | TGG |
| 25303190 | 1 | GCCCCAACACAGGGGAGAAG | TGG |
| 25303197 | 1 | CACAGGGGAGAAGTGGTTTC | AGG |
| 25303211 | 1 | GGTTTCAGGATCAGCAAAGC | AGG |
| 25303212 | 1 | GTTTCAGGATCAGCAAAGCA | GGG |
| 25303215 | 1 | TCAGGATCAGCAAAGCAGGG | AGG |
| 25303225 | 1 | CAAAGCAGGGAGGATGTTAC | AGG |
| 25303226 | 1 | AAAGCAGGGAGGATGTTACA | GGG |
| 25303241 | −1 | TGACCAGCACGCTGGGAACA | AGG |
| 25303248 | −1 | CTGCAAGTGACCAGCACGCT | GGG |
| 25303249 | 1 | TTGCCTTGTTCCCAGCGTGC | TGG |
| 25303249 | −1 | GCTGCAAGTGACCAGCACGC | TGG |
| 25303267 | 1 | GCTGGTCACTTGCAGCAAGA | TGG |
| 25303292 | −1 | GCGTGTGGGTAAAGGAAGCA | AGG |
| 25303300 | −1 | AAGAAATAGCGTGTGGGTAA | AGG |
| 25303306 | −1 | TCTGCAAAGAAATAGCGTGT | GGG |
| 25303307 | −1 | GTCTGCAAAGAAATAGCGTG | TGG |
| 25303335 | 1 | GCAGACTTATGTGCACAGTG | CGG |
| 25303341 | 1 | TTATGTGCACAGTGCGGTGT | TGG |
| 25303345 | 1 | GTGCACAGTGCGGTGTTGGC | AGG |
| 25303348 | 1 | CACAGTGCGGTGTTGGCAGG | AGG |
| 25303353 | 1 | TGCGGTGTTGGCAGGAGGCG | TGG |
| 25303359 | 1 | GTTGGCAGGAGGCGTGGCTG | TGG |
| 25303360 | 1 | TTGGCAGGAGGCGTGGCTGT | GGG |
| 25303374 | −1 | AGAAGGGATCAGGTGACACG | AGG |
| 25303384 | −1 | CAAGCCACGGAGAAGGGATC | AGG |
| 25303390 | −1 | CCATGGCAAGCCACGGAGAA | GGG |
| 25303391 | 1 | GTCACCTGATCCCTTCTCCG | TGG |
| 25303391 | −1 | ACCATGGCAAGCCACGGAGA | AGG |
| 25303397 | −1 | CCCAGCACCATGGCAAGCCA | CGG |
| 25303401 | 1 | CCCTTCTCCGTGGCTTGCCA | TGG |
| 25303407 | 1 | TCCGTGGCTTGCCATGGTGC | TGG |
| 25303407 | −1 | AGCCACAAGACCCAGCACCA | TGG |
| 25303408 | 1 | CCGTGGCTTGCCATGGTGCT | GGG |
| 25303416 | 1 | TGCCATGGTGCTGGGTCTTG | TGG |
| 25303420 | 1 | ATGGTGCTGGGTCTTGTGGC | TGG |
| 25303421 | 1 | TGGTGCTGGGTCTTGTGGCT | GGG |
| 25303435 | 1 | GTGGCTGGGCTGATCTCCGT | CGG |
| 25303436 | 1 | TGGCTGGGCTGATCTCCGTC | GGG |
| 25303437 | 1 | GGCTGGGCTGATCTCCGTCG | GGG |
| 25303438 | 1 | GCTGGGCTGATCTCCGTCGG | GGG |
| 25303440 | −1 | CAGGTACTTGGCTCCCCCGA | CGG |
| 25303452 | −1 | GTTTCTTACCGGCAGGTACT | TGG |
| 25303455 | 1 | CGGGGGAGCCAAGTACCTGC | CGG |
| 25303459 | −1 | TTGTCTAGTTTCTTACCGGC | AGG |
| 25303463 | −1 | TTAGTTGTCTAGTTTCTTAC | CGG |
| 25303486 | −1 | GCCTTCAGCCAAAGCAGAGG | AGG |
| 25303489 | 1 | ACAACTAACCTCCTCTGCTT | TGG |
| 25303489 | −1 | CTGGCCTTCAGCCAAAGCAG | AGG |
| 25303496 | 1 | ACCTCCTCTGCTTTGGCTGA | AGG |
| 25303504 | 1 | TGCTTTGGCTGAAGGCCAGC | AGG |
| 25303508 | −1 | ATCAGGTCCCAGCGTCCTGC | TGG |
| 25303511 | 1 | GCTGAAGGCCAGCAGGACGC | TGG |
| 25303512 | 1 | CTGAAGGCCAGCAGGACGCT | GGG |
| 25303521 | 1 | AGCAGGACGCTGGGACCTGA | TGG |
| 25303522 | 1 | GCAGGACGCTGGGACCTGAT | GGG |
| 25303525 | −1 | GCACTGCACAGTGGCCCATC | AGG |
| 25303534 | −1 | TGCAGCTGTGCACTGCACAG | TGG |
| 25303550 | 1 | GTGCAGTGCACAGCTGCATT | AGG |
| 25303554 | 1 | AGTGCACAGCTGCATTAGGC | AGG |
| 25303560 | 1 | CAGCTGCATTAGGCAGGTGT | CGG |
| 25303576 | 1 | GTGTCGGCGCATTCTCTTAT | TGG |
| 25303594 | 1 | ATTGGCTTCAACGCCTAGTG | AGG |
| 25303595 | 1 | TTGGCTTCAACGCCTAGTGA | GGG |
| 25303596 | −1 | GCCAGGATGGATCCCTCACT | AGG |
| 25303606 | 1 | GCCTAGTGAGGGATCCATCC | TGG |
| 25303609 | −1 | AATGCGCCACCGAGCCAGGA | TGG |
| 25303611 | 1 | GTGAGGGATCCATCCTGGCT | CGG |
| 25303613 | −1 | AACAAATGCGCCACCGAGCC | AGG |
| 25303614 | 1 | AGGGATCCATCCTGGCTCGG | TGG |
| 25303635 | 1 | GGCGCATTTGTTAAGATGCT | CGG |
| 25303636 | 1 | GCGCATTTGTTAAGATGCTC | GGG |
| 25303642 | 1 | TTGTTAAGATGCTCGGGAGC | AGG |
| 25303645 | 1 | TTAAGATGCTCGGGAGCAGG | TGG |
| 25303662 | −1 | ATGCCCAAGCAAGCTCAAAT | GGG |
| 25303663 | −1 | AATGCCCAAGCAAGCTCAAA | TGG |
| 25303669 | 1 | AGAACCCATTTGAGCTTGCT | TGG |
| 25303670 | 1 | GAACCCATTTGAGCTTGCTT | GGG |
| 25303676 | 1 | ATTTGAGCTTGCTTGGGCAT | TGG |
| 25303677 | 1 | TTTGAGCTTGCTTGGGCATT | GGG |
| 25303678 | 1 | TTGAGCTTGCTTGGGCATTG | GGG |
| 25303694 | 1 | ATTGGGGAGAATTTGTTATC | AGG |
| 25303701 | 1 | AGAATTTGTTATCAGGCTAC | TGG |
| 25303702 | 1 | GAATTTGTTATCAGGCTACT | GGG |
| 25303703 | 1 | AATTTGTTATCAGGCTACTG | GGG |
| 25303720 | 1 | CTGGGGTGTCACAGAACTCA | AGG |
| 25303725 | 1 | GTGTCACAGAACTCAAGGAC | AGG |
| 25303726 | 1 | TGTCACAGAACTCAAGGACA | GGG |
| 25303731 | 1 | CAGAACTCAAGGACAGGGAC | TGG |
| 25303741 | 1 | GGACAGGGACTGGAGTGTTG | TGG |
| 25303742 | 1 | GACAGGGACTGGAGTGTTGT | GGG |
| 25303743 | 1 | ACAGGGACTGGAGTGTTGTG | GGG |
| 25303757 | −1 | GAAGTAAAACAGGGGCTTCG | GGG |
| 25303758 | −1 | AGAAGTAAAACAGGGGCTTC | GGG |
| 25303759 | −1 | AAGAAGTAAAACAGGGGCTT | CGG |
| 25303765 | −1 | CAAAGAAAGAAGTAAAACAG | GGG |
| 25303766 | −1 | GCAAAGAAAGAAGTAAAACA | GGG |
| 25303767 | −1 | AGCAAAGAAAGAAGTAAAAC | AGG |
| 25303793 | −1 | TAAGAATAAAGCAGATATTC | AGG |
| 25303832 | −1 | ACAATGTGGGGTGAAAGAGG | AGG |
| 25303835 | −1 | CCCACAATGTGGGGTGAAAG | AGG |
| 25303844 | −1 | AGACTACACCCCACAATGTG | GGG |
| 25303845 | 1 | TCCTCTTTCACCCCACATTG | TGG |
| 25303845 | −1 | AAGACTACACCCCACAATGT | GGG |
| 25303846 | 1 | CCTCTTTCACCCCACATTGT | GGG |
| 25303846 | −1 | AAAGACTACACCCCACAATG | TGG |
| 25303847 | 1 | CTCTTTCACCCCACATTGTG | GGG |
| 25303878 | 1 | TTTGCTTCAAGAAAGCAGCC | TGG |
| 25303881 | 1 | GCTTCAAGAAAGCAGCCTGG | TGG |
| 25303885 | 1 | CAAGAAAGCAGCCTGGTGGA | tgg |
| 25303885 | −1 | gccaagagattccaTCCACC | AGG |
| 25303895 | 1 | GCCTGGTGGAtggaatctct | tgg |
| 25303907 | −1 | ctccagagaatttgggattg | ggg |
| 25303908 | −1 | tctccagagaatttgggatt | ggg |
| 25303909 | −1 | ttctccagagaatttgggat | tgg |
| 25303914 | −1 | gccccttctccagagaattt | ggg |
| 25303915 | −1 | agccccttctccagagaatt | tgg |
| 25303916 | 1 | ggccccaatcccaaattctc | tgg |
| 25303922 | 1 | aatcccaaattctctggaga | agg |
| 25303923 | 1 | atcccaaattctctggagaa | ggg |
| 25303924 | 1 | tcccaaattctctggagaag | ggg |
| 25303932 | 1 | tctctggagaaggggctctt | tgg |
| 25303942 | 1 | aggggctctttggtttaact | tgg |
| 25303962 | 1 | tggataatgttgtcttcagc | tgg |
| 25303963 | 1 | ggataatgttgtcttcagct | ggg |
| 25303964 | 1 | gataatgttgtcttcagctg | ggg |
| 25303965 | 1 | ataatgttgtcttcagctgg | ggg |
| 25303968 | 1 | atgttgtcttcagctggggg | tgg |
| 25303969 | 1 | tgttgtcttcagctgggggt | ggg |
| 25303987 | 1 | gtgggcacatcgtgcatatg | tgg |
| 25303997 | 1 | cgtgcatatgtggctgctgc | cgg |
| 25303998 | 1 | gtgcatatgtggctgctgcc | ggg |
| 25303999 | 1 | tgcatatgtggctgctgccg | ggg |
| 25304005 | −1 | acatcatccacgtggttccc | cgg |
| 25304009 | 1 | gctgctgccggggaaccacg | tgg |
| 25304013 | −1 | ctcctctcacatcatccacg | tgg |
| 25304022 | 1 | aaccacgtggatgatgtgag | agg |
| 25304040 | 1 | agaggagcagcacccagaag | agg |
| 25304041 | 1 | gaggagcagcacccagaaga | ggg |
| 25304041 | −1 | agcccagcactccctcttct | ggg |
| 25304042 | −1 | cagcccagcactccctcttc | tgg |
| 25304049 | 1 | gcacccagaagagggagtgc | tgg |
| 25304050 | 1 | cacccagaagagggagtgct | ggg |
| 25304057 | 1 | aagagggagtgctgggctga | tgg |
| 25304063 | 1 | gagtgctgggctgatggtcc | agg |
| 25304070 | −1 | AATCAGAagtggacacgacc | tgg |
| 25304081 | −1 | AAGAATTAAACAATCAGAag | tgg |
| 25304103 | 1 | TGTTTAATTCTTCTTCTAAG | TGG |
| 25304107 | 1 | TAATTCTTCTTCTAAGTGGA | TGG |
| 25304127 | −1 | GATCAGGATTTGCTGAGTAT | TGG |
| 25304143 | −1 | TGAAGTATTCTGGAACGATC | AGG |
| 25304153 | −1 | TTGGCTATAATGAAGTATTC | TGG |
| 25304168 | 1 | AATACTTCATTATAGCCAAT | TGG |
| 25304172 | −1 | AGAAGCACATTATAACCAAT | TGG |
| 25304201 | 1 | CTTCTCTAAGAGAAATATTT | AGG |
| 25304202 | 1 | TTCTCTAAGAGAAATATTTA | GGG |
| 25304219 | 1 | TTAGGGACAACAAATCTTCA | TGG |
| 25304220 | 1 | TAGGGACAACAAATCTTCAT | GGG |
| 25304236 | 1 | TCATGGGTTTGAAGACTTGA | TGG |
| 25304239 | 1 | TGGGTTTGAAGACTTGATGG | AGG |
| 25304246 | 1 | GAAGACTTGATGGAGGAAAA | AGG |
| 25304262 | 1 | AAAAAGGAGTAGATTTTCGA | AGG |
| 25304266 | 1 | AGGAGTAGATTTTCGAAGGC | TGG |
| 25304272 | 1 | AGATTTTCGAAGGCTGGATT | TGG |
| 25304281 | 1 | AAGGCTGGATTTGGATGAAC | AGG |
| 25304282 | 1 | AGGCTGGATTTGGATGAACA | GGG |
| 25304283 | 1 | GGCTGGATTTGGATGAACAG | GGG |
| 25304292 | 1 | TGGATGAACAGGGGCTATTC | AGG |
| 25304293 | 1 | GGATGAACAGGGGCTATTCA | GGG |
| 25304313 | −1 | agtttttcctaatTTTAGGT | TGG |
| 25304317 | 1 | GTGCATTCCAACCTAAAatt | agg |
| 25304317 | −1 | agccagtttttcctaatTTT | AGG |
| 25304326 | 1 | AACCTAAAattaggaaaaac | tgg |
| 25304330 | 1 | TAAAattaggaaaaactggc | tgg |
| 25304331 | 1 | AAAattaggaaaaactggct | ggg |
| 25304339 | 1 | gaaaaactggctgggcgcag | tgg |
| 25304353 | 1 | gcgcagtggctcacgcgctt | tgg |
| 25304354 | 1 | cgcagtggctcacgcgcttt | ggg |
| 25304357 | 1 | agtggctcacgcgctttggg | agg |
| 25304363 | 1 | tcacgcgctttgggaggccg | agg |
| 25304366 | 1 | cgcgctttgggaggccgagg | cgg |
| 25304367 | 1 | gcgctttgggaggccgaggc | ggg |
| 25304369 | −1 | ctcaggccatctgcccgcct | cgg |
| 25304374 | 1 | gggaggccgaggcgggcaga | tgg |
| 25304381 | 1 | cgaggcgggcagatggcctg | agg |
| 25304386 | 1 | cgggcagatggcctgaggtc | agg |
| 25304386 | −1 | ggtcttgaactcctgacctc | agg |
| 25304404 | 1 | tcaggagttcaagaccagcc | tgg |
| 25304407 | −1 | tttcaccatgttggccaggc | tgg |
| 25304411 | −1 | tgggtttcaccatgttggcc | agg |
| 25304413 | 1 | caagaccagcctggccaaca | tgg |
| 25304416 | −1 | agagatgggtttcaccatgt | tgg |
| 25304430 | −1 | tttgtacttttagtagagat | ggg |
| 25304431 | −1 | ttttgtacttttagtagaga | tgg |
| 25304452 | 1 | taaaagtacaaaaattagcc | agg |
| 25304457 | 1 | gtacaaaaattagccaggca | tgg |
| 25304459 | −1 | caggtgcccgccaccatgcc | tgg |
| 25304460 | 1 | caaaaattagccaggcatgg | tgg |
| 25304463 | 1 | aaattagccaggcatggtgg | cgg |
| 25304464 | 1 | aattagccaggcatggtggc | ggg |
| 25304478 | −1 | tcctgagtcgctaagatgac | agg |
| 25304488 | 1 | acctgtcatcttagcgactc | agg |
| 25304491 | 1 | tgtcatcttagcgactcagg | agg |
| 25304519 | 1 | acacgagaatcacttgaacc | tgg |
| 25304520 | 1 | cacgagaatcacttgaacct | ggg |
| 25304526 | −1 | cactgcaagctctgtctccc | agg |
| 25304556 | 1 | agtgagctgaaatcgtgcca | tgg |
| 25304562 | −1 | tcgcccaggctggagtgcca | tgg |
| 25304569 | 1 | cgtgccatggcactccagcc | tgg |
| 25304570 | 1 | gtgccatggcactccagcct | ggg |
| 25304572 | −1 | tcttgttctgtcgcccaggc | tgg |
| 25304576 | −1 | agagtcttgttctgtcgccc | agg |
| 25304609 | 1 | tgtcttaaaaaaaaaaaaag | tgg |
| 25304625 | 1 | aaagtggtttatatacagag | tgg |
| 25304649 | −1 | acaggatttcattcttttta | tgg |
| 25304667 | −1 | tccatgttgctgcaaatgac | agg |
| 25304677 | 1 | tcctgtcatttgcagcaaca | tgg |
| 25304681 | 1 | gtcatttgcagcaacatgga | tgg |
| 25304687 | 1 | tgcagcaacatggatggaac | tgg |
| 25304690 | 1 | agcaacatggatggaactgg | agg |
| 25304716 | 1 | ttaaaaaataaaattaaata | agg |
| 25304752 | 1 | TACTTCGATTAACCAAAACC | AGG |
| 25304753 | 1 | ACTTCGATTAACCAAAACCA | GGG |
| 25304753 | −1 | AATCAGATTTGCCCTGGTTT | TGG |
| 25304759 | −1 | GATGAAAATCAGATTTGCCC | TGG |
| 25304779 | 1 | ATCTGATTTTCATCTTTGCA | AGG |
| 25304780 | 1 | TCTGATTTTCATCTTTGCAA | GGG |
| 25304781 | 1 | CTGATTTTCATCTTTGCAAG | GGG |
| 25304806 | 1 | CAAATTTCTTTTATCTCCTC | TGG |
| 25304811 | −1 | TTTCAGGGTTTCAAAGCCAG | AGG |
| 25304826 | −1 | CCCTTCCTCCTTTCATTTCA | GGG |
| 25304827 | −1 | GCCCTTCCTCCTTTCATTTC | AGG |
| 25304829 | 1 | CTTTGAAACCCTGAAATGAA | AGG |
| 25304832 | 1 | TGAAACCCTGAAATGAAAGG | AGG |
| 25304836 | 1 | ACCCTGAAATGAAAGGAGGA | AGG |
| 25304837 | 1 | CCCTGAAATGAAAGGAGGAA | GGG |
| 25304890 | −1 | ACAAGCTCAGGGAATGCGAT | GGG |
| 25304891 | −1 | AACAAGCTCAGGGAATGCGA | TGG |
| 25304901 | −1 | AAGTCAAGGAAACAAGCTCA | GGG |
| 25304902 | −1 | GAAGTCAAGGAAACAAGCTC | AGG |
| 25304915 | −1 | TCCTGCCAGTGATGAAGTCA | AGG |
| 25304921 | 1 | TGTTTCCTTGACTTCATCAC | TGG |
| 25304925 | 1 | TCCTTGACTTCATCACTGGC | AGG |
| 25304989 | −1 | AAAACGTATGTGTtgaatga | agg |
| 25305045 | 1 | CTATAGTTTAGTGAGCGAaa | tgg |
| 25305078 | 1 | tacagtgtgagaacagcaag | agg |
| 25305079 | 1 | acagtgtgagaacagcaaga | ggg |
| 25305098 | 1 | agggcacatctgagctagcc | tgg |
| 25305099 | 1 | gggcacatctgagctagcct | ggg |
| 25305103 | 1 | acatctgagctagcctggga | tgg |
| 25305104 | 1 | catctgagctagcctgggat | ggg |
| 25305105 | −1 | agcatttccagacccatccc | agg |
| 25305109 | 1 | gagctagcctgggatgggtc | tgg |
| 25305122 | 1 | atgggtctggaaatgcttcc | tgg |
| 25305129 | −1 | tcaaccgtttcctctgctcc | agg |
| 25305130 | 1 | ggaaatgcttcctggagcag | agg |
| 25305136 | 1 | gcttcctggagcagaggaaa | cgg |
| 25305155 | −1 | actacttctctgtcaacact | tgg |
| 25305176 | 1 | acagagaagtagtattagcc | agg |
| 25305183 | −1 | acattccccatgtctctgcc | tgg |
| 25305187 | 1 | gtattagccaggcagagaca | tgg |
| 25305188 | 1 | tattagccaggcagagacat | ggg |
| 25305189 | 1 | attagccaggcagagacatg | ggg |
| 25305202 | 1 | agacatggggaatgtattcc | agg |
| 25305209 | 1 | gggaatgtattccaggcaga | agg |
| 25305209 | −1 | tacacactgtgccttctgcc | tgg |
| 25305250 | 1 | ttattgttaagaagagtgtg | tgg |
| 25305260 | 1 | gaagagtgtgtggcccaacc | agg |
| 25305262 | −1 | AGAATGTctgtttcctggtt | ggg |
| 25305263 | −1 | TAGAATGTctgtttcctggt | tgg |
| 25305267 | −1 | CCTTTAGAATGTctgtttcc | tgg |
| 25305278 | 1 | ccaggaaacagACATTCTAA | AGG |
| 25305284 | 1 | aacagACATTCTAAAGGCAT | AGG |
| 25305285 | 1 | acagACATTCTAAAGGCATA | GGG |
| 25305295 | 1 | TAAAGGCATAGGGTCCACCC | AGG |
| 25305298 | −1 | GGGTCCACCATGCTCCTGGG | TGG |
| 25305301 | −1 | TCTGGGTCCACCATGCTCCT | GGG |
| 25305302 | 1 | ATAGGGTCCACCCAGGAGCA | TGG |
| 25305302 | −1 | ATCTGGGTCCACCATGCTCC | TGG |
| 25305305 | 1 | GGGTCCACCCAGGAGCATGG | TGG |
| 25305318 | −1 | CTCCCATCTTTCAGGGATCT | GGG |
| 25305319 | −1 | CCTCCCATCTTTCAGGGATC | TGG |
| 25305325 | −1 | TGAGCACCTCCCATCTTTCA | GGG |
| 25305326 | 1 | GGACCCAGATCCCTGAAAGA | TGG |
| 25305326 | −1 | CTGAGCACCTCCCATCTTTC | AGG |
| 25305327 | 1 | GACCCAGATCCCTGAAAGAT | GGG |
| 25305330 | 1 | CCAGATCCCTGAAAGATGGG | AGG |
| 25305338 | 1 | CTGAAAGATGGGAGGTGCTC | AGG |
| 25305350 | 1 | AGGTGCTCAGGCACACTTCC | TGG |
| 25305351 | 1 | GGTGCTCAGGCACACTTCCT | GGG |
| 25305357 | −1 | CCAGACTCCTCAACTAGCCC | AGG |
| 25305361 | 1 | CACACTTCCTGGGCTAGTTG | AGG |
| 25305368 | 1 | CCTGGGCTAGTTGAGGAGTC | TGG |
| 25305437 | 1 | agagtctcattctgtcaccc | agg |
| 25305441 | 1 | tctcattctgtcacccaggc | tgg |
| 25305443 | −1 | gcaccactgcactccagcct | ggg |
| 25305444 | −1 | tgcaccactgcactccagcc | tgg |
| 25305451 | 1 | tcacccaggctggagtgcag | tgg |
| 25305484 | −1 | cacttgaacccaggaggtgg | agg |
| 25305486 | 1 | tcactgcaacctccacctcc | tgg |
| 25305487 | 1 | cactgcaacctccacctcct | ggg |
| 25305487 | −1 | aatcacttgaacccaggagg | tgg |
| 25305490 | −1 | gagaatcacttgaacccagg | agg |
| 25305493 | −1 | taggagaatcacttgaaccc | agg |
| 25305512 | −1 | gctactcaggaggctgaggt | agg |
| 25305516 | −1 | cccagctactcaggaggctg | agg |
| 25305522 | −1 | tgtaatcccagctactcagg | agg |
| 25305525 | −1 | acctgtaatcccagctactc | agg |
| 25305526 | 1 | acctcagcctcctgagtagc | tgg |
| 25305527 | 1 | cctcagcctcctgagtagct | ggg |
| 25305535 | 1 | tcctgagtagctgggattac | agg |
| 25305549 | −1 | aattagccaggcatggtggt | ggg |
| 25305550 | −1 | aaattagccaggcatggtgg | tgg |
| 25305553 | −1 | cgaaaattagccaggcatgg | tgg |
| 25305554 | 1 | caggtgcccaccaccatgcc | tgg |
| 25305556 | −1 | ACacgaaaattagccaggca | tgg |
| 25305561 | −1 | TACACACacgaaaattagcc | agg |
| 25305620 | 1 | tgttgttgttgttgttgaga | cgg |
| 25305641 | 1 | ggtgtctcgctcttttgccc | agg |
| 25305645 | 1 | tctcgctcttttgcccaggc | tgg |
| 25305647 | −1 | gcgccactgcactccagcct | ggg |
| 25305648 | −1 | ggcgccactgcactccagcc | tgg |
| 25305655 | 1 | ttgcccaggctggagtgcag | tgg |
| 25305669 | −1 | gagcttgcagtaagctgaga | tgg |
| 25305690 | 1 | ttactgcaagctccgcctcc | cgg |
| 25305691 | 1 | tactgcaagctccgcctccc | ggg |
| 25305691 | −1 | aatggtgtgaacccgggagg | cgg |
| 25305694 | −1 | gagaatggtgtgaacccggg | agg |
| 25305697 | −1 | caggagaatggtgtgaaccc | ggg |
| 25305698 | −1 | gcaggagaatggtgtgaacc | cgg |
| 25305709 | −1 | aggaggctgaggcaggagaa | tgg |
| 25305716 | −1 | gctactcaggaggctgaggc | agg |
| 25305720 | −1 | cccagctactcaggaggctg | agg |
| 25305726 | −1 | tgtagacccagctactcagg | agg |
| 25305729 | −1 | gcctgtagacccagctactc | agg |
| 25305730 | 1 | gcctcagcctcctgagtagc | tgg |
| 25305731 | 1 | cctcagcctcctgagtagct | ggg |
| 25305739 | 1 | tcctgagtagctgggtctac | agg |
| 25305753 | −1 | aattagctgggcgtggtggt | ggg |
| 25305754 | −1 | aaattagctgggcgtggtgg | tgg |
| 25305757 | −1 | aaaaaattagctgggcgtgg | tgg |
| 25305760 | −1 | cacaaaaaattagctgggcg | tgg |
| 25305765 | −1 | aaaaacacaaaaaattagct | ggg |
| 25305766 | −1 | taaaaacacaaaaaattagc | tgg |
| 25305787 | 1 | ttttgtgtttttagtagaga | cgg |
| 25305788 | 1 | tttgtgtttttagtagagac | ggg |
| 25305789 | 1 | ttgtgtttttagtagagacg | ggg |
| 25305803 | 1 | gagacggggtttcaccatgt | tgg |
| 25305806 | −1 | caagaccagcagggccaaca | tgg |
| 25305812 | 1 | tttcaccatgttggccctgc | tgg |
| 25305815 | −1 | tcgggagttcaagaccagca | ggg |
| 25305816 | −1 | gtcgggagttcaagaccagc | agg |
| 25305833 | 1 | ggtcttgaactcccgacttc | agg |
| 25305833 | −1 | tgggtggatcacctgaagtc | ggg |
| 25305834 | −1 | atgggtggatcacctgaagt | cgg |
| 25305849 | −1 | ctttgggaggccgacatggg | tgg |
| 25305850 | 1 | ttcaggtgatccacccatgt | cgg |
| 25305852 | −1 | gcactttgggaggccgacat | ggg |
| 25305853 | −1 | agcactttgggaggccgaca | tgg |
| 25305862 | −1 | tgtaatcccagcactttggg | agg |
| 25305865 | −1 | gcctgtaatcccagcacttt | ggg |
| 25305866 | 1 | atgtcggcctcccaaagtgc | tgg |
| 25305866 | −1 | tgcctgtaatcccagcactt | tgg |
| 25305867 | 1 | tgtcggcctcccaaagtgct | ggg |
| 25305875 | 1 | tcccaaagtgctgggattac | agg |
| 25305893 | −1 | AAAATCCAggttgggcacgg | tgg |
| 25305896 | −1 | ATAAAAATCCAggttgggca | cgg |
| 25305899 | 1 | atgagccaccgtgcccaacc | TGG |
| 25305901 | −1 | TCAGAATAAAAATCCAggtt | ggg |
| 25305902 | −1 | TTCAGAATAAAAATCCAggt | tgg |
| 25305906 | −1 | AGTCTTCAGAATAAAAATCC | Agg |
| 25305923 | 1 | TTTTTATTCTGAAGACTAAT | AGG |
| 25305924 | 1 | TTTTATTCTGAAGACTAATA | GGG |
| 25305933 | 1 | GAAGACTAATAGGGATTCTA | AGG |
| 25305937 | 1 | ACTAATAGGGATTCTAAGGA | AGG |
| 25305951 | −1 | ATATGCAAATTCAATCAGGC | TGG |
| 25305955 | −1 | ACACATATGCAAATTCAATC | AGG |
| 25305978 | −1 | CAGCCGTGAGCCAGCAGATG | TGG |
| 25305979 | 1 | GCATATGTGTCCACATCTGC | TGG |
| 25305986 | 1 | TGTCCACATCTGCTGGCTCA | CGG |
| 25305994 | 1 | TCTGCTGGCTCACGGCTGTG | TGG |
| 25305995 | 1 | CTGCTGGCTCACGGCTGTGT | GGG |
| 25305998 | 1 | CTGGCTCACGGCTGTGTGGG | AGG |
| 25306009 | 1 | CTGTGTGGGAGGCTGAGTGA | TGG |
| 25306010 | 1 | TGTGTGGGAGGCTGAGTGAT | GGG |
| 25306011 | 1 | GTGTGGGAGGCTGAGTGATG | GGG |
| 25306014 | 1 | TGGGAGGCTGAGTGATGGGG | AGG |
| 25306018 | 1 | AGGCTGAGTGATGGGGAGGA | AGG |
| 25306031 | 1 | GGGAGGAAGGATTACTGAGT | AGG |
| 25306032 | 1 | GGAGGAAGGATTACTGAGTA | GGG |
| 25306041 | 1 | ATTACTGAGTAGGGATCTGA | AGG |
| 25306046 | 1 | TGAGTAGGGATCTGAAGGTG | TGG |
| 25306058 | −1 | CTGGTTAGAAAGAAAGCATG | AGG |
| 25306077 | −1 | CATCCCAAAGACAACACAGC | TGG |
| 25306084 | 1 | CTAACCAGCTGTGTTGTCTT | TGG |
| 25306085 | 1 | TAACCAGCTGTGTTGTCTTT | GGG |
| 25306089 | 1 | CAGCTGTGTTGTCTTTGGGA | TGG |
| 25306102 | 1 | TTTGGGATGGTGCTTAAATT | TGG |
| 25306103 | 1 | TTGGGATGGTGCTTAAATTT | GGG |
| 25306115 | 1 | TTAAATTTGGGCTAGACCAG | TGG |
| 25306116 | 1 | TAAATTTGGGCTAGACCAGT | GGG |
| 25306120 | −1 | ggggggTGACCAAGACCCAC | TGG |
| 25306122 | 1 | TGGGCTAGACCAGTGGGTCT | TGG |
| 25306134 | 1 | GTGGGTCTTGGTCAcccccc | agg |
| 25306135 | 1 | TGGGTCTTGGTCAcccccca | ggg |
| 25306136 | 1 | GGGTCTTGGTCAccccccag | ggg |
| 25306137 | −1 | attgtaagatgtcccctggg | ggg |
| 25306138 | −1 | cattgtaagatgtcccctgg | ggg |
| 25306139 | −1 | acattgtaagatgtcccctg | ggg |
| 25306140 | −1 | gacattgtaagatgtcccct | ggg |
| 25306141 | −1 | agacattgtaagatgtcccc | tgg |
| 25306154 | 1 | aggggacatcttacaatgtc | tgg |
| 25306157 | 1 | ggacatcttacaatgtctgg | agg |
| 25306166 | 1 | acaatgtctggaggcgttct | tgg |
| 25306178 | 1 | ggcgttcttggttgacacag | tgg |
| 25306179 | 1 | gcgttcttggttgacacagt | ggs |
| 25306180 | 1 | cgttcttggttgacacagtg | ggg |
| 25306185 | 1 | ttggttgacacagtggggtg | agg |
| 25306186 | 1 | tggttgacacagtggggtga | ggg |
| 25306196 | 1 | agtggggtgagggctgctac | tgg |
| 25306206 | 1 | gggctgctactggcagctcg | tgg |
| 25306207 | 1 | ggctgctactggcagctcgt | ggg |
| 25306208 | 1 | gctgctactggcagctcgtg | ggg |
| 25306218 | 1 | gcagctcgtggggagagacc | agg |
| 25306219 | 1 | cagctcgtggggagagacca | ggg |
| 25306225 | −1 | aggatgttaagcagcatccc | tgg |
| 25306245 | −1 | ggggctgccctgtgtactgt | agg |
| 25306248 | 1 | cttaacatcctacagtacac | agg |
| 25306249 | 1 | ttaacatcctacagtacaca | ggg |
| 25306264 | −1 | ctgataattccttgtggtgg | ggg |
| 25306265 | −1 | gctgataattccttgtggtg | ggg |
| 25306266 | 1 | acagggcagcccccaccaca | agg |
| 25306266 | −1 | agctgataattccttgtggt | ggg |
| 25306267 | −1 | cagctgataattccttgtgg | tgg |
| 25306270 | −1 | tttcagctgataattccttg | tgg |
| 25306316 | −1 | gaccacactatgagtagcaa | ggG |
| 25306317 | −1 | ggaccacactatgagtagca | agg |
| 25306325 | 1 | GACccttgctactcatagtg | tgg |
| 25306338 | −1 | atgccaatgctgctggtcta | cgg |
| 25306345 | −1 | ccaggtgatgccaatgctgc | tgg |
| 25306346 | 1 | ggtccgtagaccagcagcat | tgg |
| 25306356 | 1 | ccagcagcattggcatcacc | tgg |
| 25306357 | 1 | cagcagcattggcatcacct | ggg |
| 25306363 | −1 | agcatttctaacaaggtccc | agg |
| 25306370 | −1 | gtctaacagcatttctaaca | agg |
| 25306392 | −1 | gctttagtggatgtggggtg | ggg |
| 25306393 | −1 | ggctttagtggatgtggggt | ggg |
| 25306394 | −1 | tggctttagtggatgtgggg | tgg |
| 25306397 | −1 | agctggctttagtggatgtg | ggg |
| 25306398 | −1 | gagctggctttagtggatgt | ggg |
| 25306399 | −1 | agagctggctttagtggatg | tgg |
| 25306405 | −1 | aaatgaagagctggctttag | tgg |
| 25306414 | −1 | agtttgttgaaatgaagagc | tgg |
| 25306437 | −1 | aatgtgcactcacatcatcg | ggg |
| 25306438 | −1 | gaatgtgcactcacatcatc | ggg |
| 25306439 | −1 | tgaatgtgcactcacatcat | cgg |
| 25306462 | 1 | gtgcacattcaagtctgaga | agG |
| 25306463 | 1 | tgcacattcaagtctgagaa | gGG |
| 25306474 | 1 | gtctgagaagGGCTTCTTTG | AGG |
| 25306490 | −1 | CCAAAGGGGGATGGGCACTA | AGG |
| 25306498 | −1 | CGGGGCCACCAAAGGGGGAT | GGG |
| 25306499 | −1 | CCGGGGCCACCAAAGGGGGA | TGG |
| 25306501 | 1 | CCTTAGTGCCCATCCCCCTT | TGG |
| 25306503 | −1 | GTATCCGGGGCCACCAAAGG | GGG |
| 25306504 | 1 | TAGTGCCCATCCCCCTTTGG | TGG |
| 25306504 | −1 | GGTATCCGGGGCCACCAAAG | GGG |
| 25306505 | −1 | TGGTATCCGGGGCCACCAAA | GGG |
| 25306506 | −1 | TTGGTATCCGGGGCCACCAA | AGG |
| 25306510 | 1 | CCATCCCCCTTTGGTGGCCC | CGG |
| 25306516 | −1 | TCACACACCCTTGGTATCCG | GGG |
| 25306517 | −1 | TTCACACACCCTTGGTATCC | GGG |
| 25306518 | −1 | TTTCACACACCCTTGGTATC | CGG |
| 25306519 | 1 | TTTGGTGGCCCCGGATACCA | AGG |
| 25306520 | 1 | TTGGTGGCCCCGGATACCAA | GGG |
| 25306525 | −1 | CCACCCCTTTCACACACCCT | TGG |
| 25306531 | 1 | GGATACCAAGGGTGTGTGAA | AGG |
| 25306532 | 1 | GATACCAAGGGTGTGTGAAA | GGG |
| 25306533 | 1 | ATACCAAGGGTGTGTGAAAG | GGG |
| 25306536 | 1 | CCAAGGGTGTGTGAAAGGGG | TGG |
| 25306537 | 1 | CAAGGGTGTGTGAAAGGGGT | GGG |
| 25306541 | 1 | GGTGTGTGAAAGGGGTGGGT | AGG |
| 25306542 | 1 | GTGTGTGAAAGGGGTGGGTA | GGG |
| 25306549 | 1 | AAAGGGGTGGGTAGGGAATA | TGG |
| 25306550 | 1 | AAGGGGTGGGTAGGGAATAT | GGG |
| 25306567 | −1 | GTTATTATAAGCAGATTGGC | AGG |
| 25306571 | −1 | AAGTGTTATTATAAGCAGAT | TGG |
| 25306591 | 1 | TTATAATAACACTTGTCCAC | AGG |
| 25306592 | 1 | TATAATAACACTTGTCCACA | GGG |
| 25306593 | 1 | ATAATAACACTTGTCCACAG | GGG |
| 25306596 | −1 | ACTCGGTTACAACACCCCTG | TGG |
| 25306612 | 1 | GGGGTGTTGTAACCGAGTGC | TGG |
| 25306613 | 1 | GGGTGTTGTAACCGAGTGCT | GGG |
| 25306613 | −1 | TGTGGGGAATCCCCAGCACT | CGG |
| 25306614 | 1 | GGTGTTGTAACCGAGTGCTG | GGG |
| 25306629 | −1 | TAGCCCATGATGGAGCTGTG | GGG |
| 25306630 | −1 | GTAGCCCATGATGGAGCTGT | GGG |
| 25306631 | −1 | TGTAGCCCATGATGGAGCTG | TGG |
| 25306636 | 1 | GATTCCCCACAGCTCCATCA | TGG |
| 25306637 | 1 | ATTCCCCACAGCTCCATCAT | GGG |
| 25306639 | −1 | GCTGAAGTTGTAGCCCATGA | TGG |
| 25306657 | 1 | GGGCTACAACTTCAGCTTGC | TGG |
| 25306658 | 1 | GGCTACAACTTCAGCTTGCT | GGG |
| 25306667 | 1 | TTCAGCTTGCTGGGTCTGCT | TGG |
| 25306693 | 1 | GATCATCTACATTGTGCTGC | TGG |
| 25306709 | 1 | CTGCTGGTGCTTGATACCGT | CGG |
| 25306714 | −1 | CATGCCATTGCCGGCTCCGA | CGG |
| 25306715 | 1 | GTGCTTGATACCGTCGGAGC | CGG |
| 25306721 | 1 | GATACCGTCGGAGCCGGCAA | TGG |
| 25306723 | −1 | AGTGACCCACATGCCATTGC | CGG |
| 25306728 | 1 | TCGGAGCCGGCAATGGCATG | TGG |
| 25306729 | 1 | CGGAGCCGGCAATGGCATGT | GGG |
| 25306736 | 1 | GGCAATGGCATGTGGGTCAC | TGG |
| 25306737 | 1 | GCAATGGCATGTGGGTCACT | GGG |
| 25306753 | −1 | GGGAGTGTTAAGGGGATGGG | GGG |
| 25306754 | −1 | GGGGAGTGTTAAGGGGATGG | GGG |
| 25306755 | −1 | AGGGGAGTGTTAAGGGGATG | GGG |
| 25306756 | −1 | GAGGGGAGTGTTAAGGGGAT | GGG |
| 25306757 | −1 | GGAGGGGAGTGTTAAGGGGA | TGG |
| 25306761 | −1 | AGTTGGAGGGGAGTGTTAAG | GGG |
| 25306762 | −1 | GAGTTGGAGGGGAGTGTTAA | GGG |
| 25306763 | −1 | TGAGTTGGAGGGGAGTGTTA | AGG |
| 25306773 | −1 | CATTTCTTCCTGAGTTGGAG | GGG |
| 25306774 | −1 | ACATTTCTTCCTGAGTTGGA | GGG |
| 25306775 | −1 | CACATTTCTTCCTGAGTTGG | AGG |
| 25306776 | 1 | TTAACACTCCCCTCCAACTC | AGG |
| 25306778 | −1 | GCACACATTTCTTCCTGAGT | TGG |
| 25306804 | 1 | ATGTGTGCAGAGTCCTTAGC | TGG |
| 25306805 | 1 | TGTGTGCAGAGTCCTTAGCT | GGG |
| 25306806 | 1 | GTGTGCAGAGTCCTTAGCTG | GGG |
| 25306806 | −1 | GAGTGCACACGCCCCAGCTA | AGG |
| 25306819 | 1 | TTAGCTGGGGCGTGTGCACT | CGG |
| 25306820 | 1 | TAGCTGGGGCGTGTGCACTC | GGG |
| 25306821 | 1 | AGCTGGGGCGTGTGCACTCG | GGG |
| 25306826 | 1 | GGGCGTGTGCACTCGGGGCC | AGG |
| 25306833 | −1 | ACCGAAGCCTACTGAGCACC | TGG |
| 25306837 | 1 | CTCGGGGCCAGGTGCTCAGT | AGG |
| 25306843 | 1 | GCCAGGTGCTCAGTAGGCTT | CGG |
| 25306857 | 1 | AGGCTTCGGTGAATATTTGT | TGG |
| 25306891 | −1 | AATCCATCCAAGGTAGGGGC | TGG |
| 25306895 | 1 | ATTCTGTCCAGCCCCTACCT | TGG |
| 25306895 | −1 | GATAAATCCATCCAAGGTAG | GGG |
| 25306896 | −1 | TGATAAATCCATCCAAGGTA | GGG |
| 25306897 | −1 | GTGATAAATCCATCCAAGGT | AGG |
| 25306899 | 1 | TGTCCAGCCCCTACCTTGGA | TGG |
| 25306901 | −1 | AGAGGTGATAAATCCATCCA | AGG |
| 25306917 | 1 | GATGGATTTATCACCTCTCC | AGG |
| 25306919 | −1 | AAAGAAGAGGTGGCCTGGAG | AGG |
| 25306924 | −1 | TTTGGAAAGAAGAGGTGGCC | TGG |
| 25306929 | −1 | CCCTATTTGGAAAGAAGAGG | TGG |
| 25306932 | −1 | TGGCCCTATTTGGAAAGAAG | AGG |
| 25306939 | 1 | GCCACCTCTTCTTTCCAAAT | AGG |
| 25306940 | 1 | CCACCTCTTCTTTCCAAATA | GGG |
| 25306942 | −1 | TATACCTAGGTGGCCCTATT | TGG |
| 25306949 | 1 | CTTTCCAAATAGGGCCACCT | AGG |
| 25306952 | −1 | GTCTTTGGTCTATACCTAGG | TGG |
| 25306955 | −1 | CGTGTCTTTGGTCTATACCT | AGG |
| 25306967 | −1 | CACAAAAGATTTCGTGTCTT | TGG |
| 25306992 | −1 | TTGACCTGCTCTGTGTTTGT | GGG |
| 25306993 | −1 | TTTGACCTGCTCTGTGTTTG | TGG |
| 25306999 | 1 | TGATCCCACAAACACAGAGC | AGG |
| 25307008 | 1 | AAACACAGAGCAGGTCAAAT | AGG |
| 25307020 | −1 | ACCACAGTCTCAATTGGCTT | GGG |
| 25307021 | −1 | AACCACAGTCTCAATTGGCT | TGG |
| 25307026 | −1 | ACCTGAACCACAGTCTCAAT | TGG |
| 25307030 | 1 | GCCCAAGCCAATTGAGACTG | TGG |
| 25307036 | 1 | GCCAATTGAGACTGTGGTTC | AGG |
| 25307060 | 1 | CGTGATGCAGAGCTTTGCTG | TGG |
| 25307079 | −1 | atgcccagctagtacgcagt | ggg |
| 25307080 | −1 | catgcccagctagtacgcag | tgg |
| 25307086 | 1 | TGctcccactgcgtactagc | tgg |
| 25307087 | 1 | Gctcccactgcgtactagct | ggg |
| 25307094 | 1 | ctgcgtactagctgggcatg | tgg |
| 25307111 | −1 | ggggcgactgaggctgagaa | agg |
| 25307121 | −1 | catttacaatggggcgactg | agg |
| 25307130 | −1 | cattatctccatttacaatg | ggg |
| 25307131 | −1 | tcattatctccatttacaat | ggg |
| 25307132 | −1 | atcattatctccatttacaa | tgg |
| 25307133 | 1 | ctcagtcgccccattgtaaa | tgg |
| 25307161 | 1 | atgatactatctcccctcac | agg |
| 25307162 | −1 | catcccaacagtcctgtgag | ggg |
| 25307163 | −1 | gcatcccaacagtcctgtga | ggg |
| 25307164 | −1 | agcatcccaacagtcctgtg | agg |
| 25307169 | 1 | atctcccctcacaggactgt | tgg |
| 25307170 | 1 | tctcccctcacaggactgtt | ggg |
| 25307180 | 1 | caggactgttgggatgctac | tgg |
| 25307200 | 1 | tggatttaataagctaatgc | agg |
| 25307201 | 1 | ggatttaataagctaatgca | ggg |
| 25307228 | −1 | CCTCTCTGGGCCTCAGGGAT | GGG |
| 25307229 | 1 | ctaagcacaACCCATCCCTG | AGG |
| 25307229 | −1 | CCCTCTCTGGGCCTCAGGGA | TGG |
| 25307233 | −1 | CCACCCCTCTCTGGGCCTCA | GGG |
| 25307234 | −1 | CCCACCCCTCTCTGGGCCTC | AGG |
| 25307239 | 1 | CCCATCCCTGAGGCCCAGAG | AGG |
| 25307240 | 1 | CCATCCCTGAGGCCCAGAGA | GGG |
| 25307241 | 1 | CATCCCTGAGGCCCAGAGAG | GGG |
| 25307241 | −1 | GCCAAGGCCCACCCCTCTCT | GGG |
| 25307242 | −1 | AGCCAAGGCCCACCCCTCTC | TGG |
| 25307244 | 1 | CCCTGAGGCCCAGAGAGGGG | TGG |
| 25307245 | 1 | CCTGAGGCCCAGAGAGGGGT | GGG |
| 25307251 | 1 | GCCCAGAGAGGGGTGGGCCT | TGG |
| 25307257 | 1 | AGAGGGGTGGGCCTTGGCTG | AGG |
| 25307257 | −1 | TCGCAGTGAGACCTCAGCCA | AGG |
| 25307270 | 1 | TTGGCTGAGGTCTCACTGCG | AGG |
| 25307273 | 1 | GCTGAGGTCTCACTGCGAGG | TGG |
| 25307274 | 1 | CTGAGGTCTCACTGCGAGGT | GGG |
| 25307281 | 1 | CTCACTGCGAGGTGGGAATG | TGG |
| 25307282 | 1 | TCACTGCGAGGTGGGAATGT | GGG |
| 25307294 | −1 | AGGACCTACCTCTGGTCTGG | AGG |
| 25307297 | 1 | AATGTGGGCCTCCAGACCAG | AGG |
| 25307297 | −1 | CACAGGACCTACCTCTGGTC | TGG |
| 25307301 | 1 | TGGGCCTCCAGACCAGAGGT | AGG |
| 25307302 | −1 | GGGGCCACAGGACCTACCTC | TGG |
| 25307309 | 1 | CAGACCAGAGGTAGGTCCTG | TGG |
| 25307314 | −1 | GTCCACTGTCTAGGGGCCAC | AGG |
| 25307321 | −1 | CATTGCTGTCCACTGTCTAG | GGG |
| 25307322 | −1 | CCATTGCTGTCCACTGTCTA | GGG |
| 25307323 | 1 | GTCCTGTGGCCCCTAGACAG | TGG |
| 25307323 | −1 | ACCATTGCTGTCCACTGTCT | AGG |
| 25307333 | 1 | CCCTAGACAGTGGACAGCAA | TGG |
| 25307358 | −1 | GGAAGTAATGGCTAGGGCTC | TGG |
| 25307364 | −1 | CATCCAGGAAGTAATGGCTA | GGG |
| 25307365 | −1 | ACATCCAGGAAGTAATGGCT | AGG |
| 25307370 | −1 | ACACAACATCCAGGAAGTAA | TGG |
| 25307372 | 1 | GAGCCCTAGCCATTACTTCC | TGG |
| 25307427 | 1 | TATAAAATGAAAAAGTGAAT | TGG |
| 25307428 | 1 | ATAAAATGAAAAAGTGAATT | GGG |
| 25307439 | 1 | AAGTGAATTGGGCACGATAC | AGG |
| 25307440 | 1 | AGTGAATTGGGCACGATACA | GGG |
| 25307463 | 1 | ATAGATTTTTAGAGATGAAC | TGG |
| 25307530 | 1 | attgactgctttaaaagtgt | tgg |
| 25307531 | 1 | ttgactgctttaaaagtgtt | ggg |
| 25307557 | −1 | caaggagataatgcatataa | tgg |
| 25307575 | −1 | taggcggttgtgagaattca | agg |
| 25307591 | −1 | tctgagaatacctcagtagg | cgg |
| 25307592 | 1 | attctcacaaccgcctactg | agg |
| 25307594 | −1 | gagtctgagaatacctcagt | agg |
| 25307642 | 1 | taagagaagttatctgccca | agg |
| 25307647 | −1 | ggttccagccgagtgacctt | ggg |
| 25307648 | −1 | aggttccagccgagtgacct | tgg |
| 25307650 | 1 | gttatctgcccaaggtcact | cgg |
| 25307654 | 1 | tctgcccaaggtcactcggc | tgg |
| 25307661 | 1 | aaggtcactcggctggaacc | tgg |
| 25307668 | −1 | CTTCAGCCATTTTTACAgcc | agg |
| 25307673 | 1 | ctggaacctggcTGTAAAAA | TGG |
| 25307683 | 1 | gcTGTAAAAATGGCTGAAGC | AGG |
| 25307691 | 1 | AATGGCTGAAGCAGGTGATG | AGG |
| 25307706 | 1 | TGATGAGGAGCTGATGCGTT | TGG |
| 25307728 | 1 | GACGTGTCTCAGAGAAATCA | TGG |
| 25307731 | 1 | GTGTCTCAGAGAAATCATGG | AGG |
| 25307739 | 1 | GAGAAATCATGGAGGCGCTG | CGG |
| 25307749 | 1 | GGAGGCGCTGCGGTTCCTAC | CGG |
| 25307753 | −1 | GAAGGCATCCAAGAACCGGT | AGG |
| 25307756 | 1 | CTGCGGTTCCTACCGGTTCT | TGG |
| 25307757 | −1 | TGTAGAAGGCATCCAAGAAC | CGG |
| 25307771 | −1 | GCTATGGTTGTCTCTGTAGA | AGG |
| 25307787 | −1 | ATCCCTATAATTTGGGGCTA | TGG |
| 25307793 | −1 | TATGTGATCCCTATAATTTG | GGG |
| 25307794 | −1 | ATATGTGATCCCTATAATTT | GGG |
| 25307795 | 1 | CAACCATAGCCCCAAATTAT | AGG |
| 25307795 | −1 | GATATGTGATCCCTATAATT | TGG |
| 25307796 | 1 | AACCATAGCCCCAAATTATA | GGG |
| 25307811 | 1 | TTATAGGGATCACATATCAG | TGG |
| 25307812 | 1 | TATAGGGATCACATATCAGT | GGG |
| 25307830 | 1 | GTGGGTGAGACATCCTTGCT | TGG |
| 25307831 | 1 | TGGGTGAGACATCCTTGCTT | GGG |
| 25307832 | −1 | TCCCCTCCTCATCCCAAGCA | AGG |
| 25307837 | 1 | AGACATCCTTGCTTGGGATG | AGG |
| 25307840 | 1 | CATCCTTGCTTGGGATGAGG | AGG |
| 25307841 | 1 | ATCCTTGCTTGGGATGAGGA | GGG |
| 25307842 | 1 | TCCTTGCTTGGGATGAGGAG | GGG |
| 25307862 | 1 | GGGATGAGCTGTGTGAAGCA | AGG |
| 25307876 | 1 | GAAGCAAGGCGCCTCTGTGA | tgg |
| 25307876 | −1 | atcactggaacccaTCACAG | AGG |
| 25307877 | 1 | AAGCAAGGCGCCTCTGTGAt | ggg |
| 25307891 | −1 | gacagtggcagacacatcac | tgg |
| 25307906 | −1 | ttgcacagttattaagacag | tgg |
| 25307941 | −1 | ctcaggcccagagacaggaa | agg |
| 25307945 | 1 | agcagaacctttcctgtctc | tgg |
| 25307946 | 1 | gcagaacctttcctgtctct | ggg |
| 25307946 | −1 | gaactctcaggcccagagac | agg |
| 25307958 | −1 | tctttcagaggggaactctc | agg |
| 25307968 | −1 | caagtcctcatctttcagag | ggg |
| 25307969 | −1 | tcaagtcctcatctttcaga | ggg |
| 25307970 | −1 | gtcaagtcctcatctttcag | agg |
| 25307974 | 1 | gagttcccctctgaaagatg | agg |
| 25307990 | 1 | gatgaggacttgacctagCA | AGG |
| 25307992 | −1 | CATGTGAGTAGGACCTTGct | agg |
| 25308003 | −1 | TTCTCTACAGGCATGTGAGT | AGG |
| 25308015 | −1 | TTCCCCTGCCTGTTCTCTAC | AGG |
| 25308018 | 1 | CTCACATGCCTGTAGAGAAC | AGG |
| 25308022 | 1 | CATGCCTGTAGAGAACAGGC | AGG |
| 25308023 | 1 | ATGCCTGTAGAGAACAGGCA | GGG |
| 25308024 | 1 | TGCCTGTAGAGAACAGGCAG | GGG |
| 25308054 | 1 | aaaaaaaaaaaaGCCAGTGA | AGG |
| 25308056 | −1 | gaagagcTCCCTTCCTTCAC | TGG |
| 25308058 | 1 | aaaaaaaaGCCAGTGAAGGA | AGG |
| 25308059 | 1 | aaaaaaaGCCAGTGAAGGAA | GGG |
| 25308087 | −1 | ggtccctgcactgtgatgat | ggg |
| 25308088 | −1 | gggtccctgcactgtgatga | tgg |
| 25308094 | 1 | tgcacccatcatcacagtgc | agg |
| 25308095 | 1 | gcacccatcatcacagtgca | ggg |
| 25308102 | 1 | tcatcacagtgcagggaccc | agg |
| 25308108 | −1 | gatctggcaacactgagcct | ggg |
| 25308109 | −1 | ggatctggcaacactgagcc | tgg |
| 25308124 | −1 | tcttgagaagtcattggatc | tgg |
| 25308130 | −1 | ttgagctcttgagaagtcat | tgg |
| 25308177 | 1 | gcatgtgctctcccaagtac | tgg |
| 25308177 | −1 | tgaattttctgccagtactt | ggg |
| 25308178 | −1 | ttgaattttctgccagtact | tgg |
| 25308211 | 1 | agattgttagtaacactgtg | tgg |
| 25308228 | 1 | gtgtggctaaaTTCTGCTTG | TGG |
| 25308229 | 1 | tgtggctaaaTTCTGCTTGT | GGG |
| 25308244 | −1 | AATCACAGAATTGGGAATCT | AGG |
| 25308252 | −1 | aaccacAGAATCACAGAATT | GGG |
| 25308253 | −1 | gaaccacAGAATCACAGAAT | TGG |
| 25308261 | 1 | TTCCCAATTCTGTGATTCTg | tgg |
| 25308269 | 1 | TCTGTGATTCTgtggttctc | tgg |
| 25308278 | 1 | CTgtggttctctggaagcat | tgg |
| 25308294 | −1 | tccaagtgatgcaggtgctg | tgg |
| 25308302 | −1 | aacaagtttccaagtgatgc | agg |
| 25308304 | 1 | tccacagcacctgcatcact | tgg |
| 25308336 | 1 | agaaatgcaagccctaccta | cgg |
| 25308336 | −1 | ctggggtggggccgtaggta | ggg |
| 25308337 | −1 | tctggggtggggccgtaggt | agg |
| 25308341 | −1 | taggtctggggtggggccgt | agg |
| 25308348 | −1 | aactgggtaggtctggggtg | ggg |
| 25308349 | −1 | taactgggtaggtctggggt | ggg |
| 25308350 | −1 | ctaactgggtaggtctgggg | tgg |
| 25308353 | −1 | tttctaactgggtaggtctg | ggg |
| 25308354 | −1 | atttctaactgggtaggtct | ggg |
| 25308355 | −1 | gatttctaactgggtaggtc | tgg |
| 25308360 | −1 | ccccagatttctaactgggt | agg |
| 25308364 | −1 | ccacccccagatttctaact | ggg |
| 25308365 | −1 | cccacccccagatttctaac | tgg |
| 25308369 | 1 | gacctacccagttagaaatc | tgg |
| 25308370 | 1 | acctacccagttagaaatct | ggg |
| 25308371 | 1 | cctacccagttagaaatctg | ggg |
| 25308372 | 1 | ctacccagttagaaatctgg | ggg |
| 25308375 | 1 | cccagttagaaatctggggg | tgg |
| 25308376 | 1 | ccagttagaaatctgggggt | ggg |
| 25308389 | −1 | ttgttcaaacatggactgat | agg |
| 25308398 | −1 | ttgtggggcttgttcaaaca | tgg |
| 25308413 | −1 | cttgcaagagaacacttgtg | ggg |
| 25308414 | −1 | gcttgcaagagaacacttgt | ggg |
| 25308415 | −1 | agcttgcaagagaacacttg | tgg |
| 25308450 | −1 | CTTTTTTGGCTATAGGTcag | tgg |
| 25308457 | −1 | GCTTTTTCTTTTTTGGCTAT | AGG |
| 25308464 | −1 | ctgATTGGCTTTTTCTTTTT | TGG |
| 25308478 | 1 | AAAAAGAAAAAGCCAATcag | tgg |
| 25308479 | −1 | tttaccagaaaaccactgAT | TGG |
| 25308486 | 1 | AAAGCCAATcagtggttttc | tgg |
| 25308492 | 1 | AATcagtggttttctggtaa | agg |
| 25308510 | 1 | aaaggattaacttaacaaac | tgg |
| 25308526 | −1 | caatcaaggctttattttct | tgg |
| 25308538 | 1 | caagaaaataaagccttgat | tgg |
| 25308540 | −1 | attgcaagtgctaccaatca | agg |
| 25308559 | 1 | ggtagcacttgcaatttcta | tgg |
| 25308582 | −1 | cagcttgaactcagtcatgc | ggg |
| 25308583 | −1 | acagcttgaactcagtcatg | cgg |
| 25308599 | 1 | tgactgagttcaagctgtca | agg |
| 25308617 | 1 | caaggagacatcactataca | tgg |
| 25308623 | 1 | gacatcactatacatggact | tgg |
| 25308624 | 1 | acatcactatacatggactt | ggg |
| 25308655 | −1 | ccagttcccataggctcagt | ggg |
| 25308656 | −1 | gccagttcccataggctcag | tgg |
| 25308659 | 1 | caatcagcccactgagccta | tgg |
| 25308660 | 1 | aatcagcccactgagcctat | ggg |
| 25308664 | −1 | gtgctggagccagttcccat | agg |
| 25308666 | 1 | cccactgagcctatgggaac | tgg |
| 25308680 | −1 | GTTGACTTGcagggatgtgc | tgg |
| 25308689 | −1 | CTGATGAGAGTTGACTTGca | ggg |
| 25308690 | −1 | CCTGATGAGAGTTGACTTGc | agg |
| 25308701 | 1 | cctgCAAGTCAACTCTCATC | AGG |
| 25308702 | 1 | ctgCAAGTCAACTCTCATCA | GGG |
| 25308716 | 1 | TCATCAGGGTGAGTGAGTTG | AGG |
| 25308729 | −1 | GCAAGAGGATAACTGCTTCT | TGG |
| 25308744 | −1 | CTGGGTCCTGCAAAGGCAAG | AGG |
| 25308749 | 1 | AGTTATCCTCTTGCCTTTGC | AGG |
| 25308751 | −1 | CCTTTGCCTGGGTCCTGCAA | AGG |
| 25308756 | 1 | CTCTTGCCTTTGCAGGACCC | AGG |
| 25308762 | 1 | CCTTTGCAGGACCCAGGCAA | AGG |
| 25308762 | −1 | CTATGCCCTTCCCTTTGCCT | GGG |
| 25308763 | 1 | CTTTGCAGGACCCAGGCAAA | GGG |
| 25308763 | −1 | ACTATGCCCTTCCCTTTGCC | TGG |
| 25308767 | 1 | GCAGGACCCAGGCAAAGGGA | AGG |
| 25308768 | 1 | CAGGACCCAGGCAAAGGGAA | GGG |
| 25308797 | 1 | GACAGTGATGATCTCTCTTC | CGG |
| 25308805 | −1 | ctcagCAAACCAAAGACTTC | CGG |
| 25308807 | 1 | ATCTCTCTTCCGGAAGTCTT | TGG |
| 25308825 | 1 | TTTGGTTTGctgagagtaaa | agg |
| 25308830 | 1 | TTTGctgagagtaaaaggcg | tgg |
| 25308831 | 1 | TTGctgagagtaaaaggcgt | ggg |
| 25308843 | 1 | aaaggcgtgggcttcaccag | tgg |
| 25308848 | −1 | tgcatgactggcttcaccac | tgg |
| 25308860 | −1 | caggactaaggctgcatgac | tgg |
| 25308872 | 1 | cagtcatgcagccttagtcc | tgg |
| 25308872 | −1 | gagtttcagtaccaggacta | agg |
| 25308879 | −1 | atttagagagtttcagtacc | agg |
| 25308914 | 1 | tcagttttctatctgtaaaa | tgg |
| 25308915 | 1 | cagttttctatctgtaaaat | ggg |
| 25308936 | −1 | gcacagcaaccctgtgacat | agg |
| 25308937 | 1 | gaaaataagacctatgtcac | agg |
| 25308938 | 1 | aaaataagacctatgtcaca | ggg |
| 25308980 | −1 | ATCAGTCATCATAAAGAACG | GGG |
| 25308981 | −1 | CATCAGTCATCATAAAGAAC | GGG |
| 25308982 | −1 | GCATCAGTCATCATAAAGAA | CGG |
| 25309008 | 1 | ACTGATGCTGCATCCGTATG | AGG |
| 25309010 | −1 | TACATAGAGATGTCCTCATA | CGG |
| 25309025 | 1 | ATGAGGACATCTCTATGTAA | TGG |
| 25309033 | 1 | ATCTCTATGTAATGGAAAGA | TGG |
| 25309040 | 1 | TGTAATGGAAAGATGGAGAG | AGG |
| 25309069 | 1 | CGCAAAGTCACAACACTTAA | TGG |
| 25309070 | 1 | GCAAAGTCACAACACTTAAT | GGG |
| 25309078 | 1 | ACAACACTTAATGGGAACTG | TGG |
| 25309091 | 1 | GGAACTGTGGATTAGCTACT | TGG |
| 25309094 | 1 | ACTGTGGATTAGCTACTTGG | TGG |
| 25309100 | 1 | GATTAGCTACTTGGTGGCAT | TGG |
| 25309101 | 1 | ATTAGCTACTTGGTGGCATT | GGG |
| 25309138 | −1 | AAATTGGGAAATATTGTTTG | TGG |
| 25309153 | −1 | GCTCATCTGAATAGGAAATT | GGG |
| 25309154 | −1 | TGCTCATCTGAATAGGAAAT | TGG |
| 25309161 | −1 | TCACATATGCTCATCTGAAT | AGG |
| 25309201 | 1 | CAGATGCTGTGATCAGAACC | AGG |
| 25309205 | 1 | TGCTGTGATCAGAACCAGGA | TGG |
| 25309208 | −1 | TTGTGGGAAATGCTCCATCC | TGG |
| 25309224 | −1 | TTAAAAATCCCACAGTTTGT | GGG |
| 25309225 | −1 | CTTAAAAATCCCACAGTTTG | TGG |
| 25309226 | 1 | GGAGCATTTCCCACAAACTG | TGG |
| 25309227 | 1 | GAGCATTTCCCACAAACTGT | GGG |
| 25309242 | 1 | ACTGTGGGATTTTTAAGTAA | TGG |
| 25309243 | 1 | CTGTGGGATTTTTAAGTAAT | GGG |
| 25309247 | 1 | GGGATTTTTAAGTAATGGGA | AGG |
| 25309260 | 1 | AATGGGAAGGCACACTGaaa | tgg |
| 25309315 | −1 | tttctccctgacgtaatcaa | agg |
| 25309320 | 1 | ctcagtcctttgattacgtc | agg |
| 25309321 | 1 | tcagtcctttgattacgtca | ggg |
| 25309343 | 1 | gagaaaagaaagtccccact | tgg |
| 25309345 | −1 | agagattctcaggccaagtg | ggg |
| 25309346 | −1 | cagagattctcaggccaagt | ggg |
| 25309347 | −1 | gcagagattctcaggccaag | tgg |
| 25309355 | −1 | agaagggtgcagagattctc | agg |
| 25309371 | −1 | gtggttaacaagagctagaa | ggg |
| 25309372 | −1 | agtggttaacaagagctaga | agg |
| 25309390 | −1 | ttctctgctattcaaaagag | tgg |
| 25309415 | −1 | ctcccagatatggcagtctg | agg |
| 25309423 | 1 | aaacctcagactgccatatc | tgg |
| 25309424 | 1 | aacctcagactgccatatct | ggg |
| 25309425 | −1 | gctaaaatctctcccagata | tgg |
| 25309484 | −1 | tgaaatagaagggaaatggg | agg |
| 25309487 | −1 | gcttgaaatagaagggaaat | ggg |
| 25309488 | −1 | agcttgaaatagaagggaaa | tgg |
| 25309494 | −1 | gttactagcttgaaatagaa | ggg |
| 25309495 | −1 | agttactagcttgaaataga | agg |
| 25309556 | 1 | aatgtaaaaataagtctatt | tgg |
| 25309584 | 1 | aaaaattttaatagcatctc | tgg |
| 25309597 | 1 | gcatctctggaatgccagta | tgg |
| 25309600 | −1 | attcatgaatttagccatac | tgg |
| 25309628 | −1 | ttcccagatttcagcatttg | agg |
| 25309636 | 1 | tgtcctcaaatgctgaaatc | tgg |
| 25309637 | 1 | gtcctcaaatgctgaaatct | ggg |
| 25309647 | 1 | gctgaaatctgggaagcaTC | TGG |
| 25309659 | −1 | gcaggcctgtccacaaagct | tgG |
| 25309660 | 1 | aagcaTCTGGCcaagctttg | tgg |
| 25309665 | 1 | TCTGGCcaagctttgtggac | agg |
| 25309677 | −1 | tcttgggattcaaactaggc | agg |
| 25309681 | −1 | tggctcttgggattcaaact | agg |
| 25309693 | −1 | gcttggactgggtggctctt | ggg |
| 25309694 | −1 | ggcttggactgggtggctct | tgg |
| 25309701 | −1 | gttttgtggcttggactggg | tgg |
| 25309704 | −1 | aatgttttgtggcttggact | ggg |
| 25309705 | −1 | caatgttttgtggcttggac | tgg |
| 25309710 | −1 | aattccaatgttttgtggct | tgg |
| 25309715 | −1 | ccaagaattccaatgttttg | tgg |
| 25309717 | 1 | cagtccaagccacaaaacat | tgg |
| 25309726 | 1 | ccacaaaacattggaattct | tgg |
| 25309745 | −1 | cagagggcaagttcaggtta | ggg |
| 25309746 | −1 | acagagggcaagttcaggtt | agg |
| 25309751 | −1 | atttcacagagggcaagttc | agg |
| 25309761 | −1 | tagtgtccctatttcacaga | ggg |
| 25309762 | −1 | ttagtgtccctatttcacag | agg |
| 25309765 | 1 | gaacttgccctctgtgaaat | agg |
| 25309766 | 1 | aacttgccctctgtgaaata | ggg |
| 25309788 | 1 | gacactaatagctcactcac | agg |
| 25309789 | 1 | acactaatagctcactcaca | ggg |
| 25309800 | 1 | tcactcacagggctgctgtg | agg |
| 25309818 | 1 | tgaggaCATGTGTTGAGCTG | AGG |
| 25309819 | 1 | gaggaCATGTGTTGAGCTGA | GGG |
| 25309829 | 1 | GTTGAGCTGAGGGTCTCGCC | AGG |
| 25309830 | 1 | TTGAGCTGAGGGTCTCGCCA | GGG |
| 25309831 | 1 | TGAGCTGAGGGTCTCGCCAG | GGG |
| 25309836 | −1 | TCCCTGCACAGGGTCTCCCC | TGG |
| 25309845 | 1 | CGCCAGGGGAGACCCTGTGC | AGG |
| 25309846 | 1 | GCCAGGGGAGACCCTGTGCA | GGG |
| 25309846 | −1 | GATAACAGTCTCCCTGCACA | GGG |
| 25309847 | −1 | TGATAACAGTCTCCCTGCAC | AGG |
| 25309861 | 1 | GTGCAGGGAGACTGTTATCA | TGG |
| 25309867 | 1 | GGAGACTGTTATCATGGTGA | TGG |
| 25309904 | −1 | TCATTCTATATGATGCTGTC | TGG |
| 25309922 | 1 | GCATCATATAGAATGAGTTG | TGG |
| 25309923 | 1 | CATCATATAGAATGAGTTGT | GGG |
| 25309924 | 1 | ATCATATAGAATGAGTTGTG | GGG |
| 25309927 | 1 | ATATAGAATGAGTTGTGGGG | TGG |
| 25309938 | 1 | GTTGTGGGGTGGCAGTCAGC | AGG |
| 25309943 | 1 | GGGGTGGCAGTCAGCAGGTT | TGG |
| 25309944 | 1 | GGGTGGCAGTCAGCAGGTTT | GGG |
| 25309961 | −1 | AGTAATAAGTGGCAGAATAG | AGG |
| 25309972 | −1 | gggtttttttAAGTAATAAG | TGG |
| 25309992 | −1 | tATATAAGTTGGGttttttg | ggg |
| 25309993 | −1 | ctATATAAGTTGGGtttttt | ggg |
| 25309994 | −1 | actATATAAGTTGGGttttt | tgg |
| 25310002 | −1 | tagcttatactATATAAGTT | GGG |
| 25310003 | −1 | atagcttatactATATAAGT | TGG |
| 25310028 | −1 | gtatgatatttgcacttttc | tgg |
| 25310056 | −1 | atatcagaagattcatcaaa | tgg |
| 25310079 | −1 | Ttctgggtgttggttatgtg | ggg |
| 25310080 | −1 | GTtctgggtgttggttatgt | ggg |
| 25310081 | −1 | GGTtctgggtgttggttatg | tgg |
| 25310089 | −1 | CAAGAAGAGGTtctgggtgt | tgg |
| 25310095 | −1 | ATGAGACAAGAAGAGGTtct | ggg |
| 25310096 | −1 | AATGAGACAAGAAGAGGTtc | tgg |
| 25310102 | −1 | tcctGGAATGAGACAAGAAG | AGG |
| 25310112 | 1 | ACCTCTTCTTGTCTCATTCC | agg |
| 25310119 | −1 | agtcaggttagtggttatcc | tGG |
| 25310128 | −1 | gctgttagaagtcaggttag | tgg |
| 25310135 | −1 | gactgatgctgttagaagtc | agg |
| 25310182 | 1 | tttgtacattatataTGTGa | tgg |
| 25310205 | −1 | ttccagcacatgaaatttgg | ggg |
| 25310206 | −1 | tttccagcacatgaaatttg | ggg |
| 25310207 | −1 | gtttccagcacatgaaattt | ggg |
| 25310208 | −1 | agtttccagcacatgaaatt | tgg |
| 25310214 | 1 | gtcccccaaatttcatgtgc | tgg |
| 25310235 | −1 | accatcaacatatgaattga | agg |
| 25310245 | 1 | tccttcaattcatatgttga | tgg |
| 25310252 | 1 | attcatatgttgatggtttt | tgg |
| 25310255 | 1 | catatgttgatggtttttgg | agg |
| 25310259 | 1 | tgttgatggtttttggagga | agg |
| 25310260 | 1 | gttgatggtttttggaggaa | ggg |
| 25310267 | 1 | gtttttggaggaagggcctt | tgg |
| 25310268 | 1 | tttttggaggaagggccttt | ggg |
| 25310272 | −1 | taatcctaattacttcccaa | agg |
| 25310279 | 1 | agggcctttgggaagtaatt | agg |
| 25310290 | 1 | gaagtaattaggattagata | agg |
| 25310296 | 1 | attaggattagataaggtca | tgg |
| 25310297 | 1 | ttaggattagataaggtcat | ggg |
| 25310298 | 1 | taggattagataaggtcatg | ggg |
| 25310303 | 1 | ttagataaggtcatggggtg | agg |
| 25310311 | 1 | ggtcatggggtgaggtatga | tgg |
| 25310317 | 1 | ggggtgaggtatgatggcac | tgg |
| 25310352 | 1 | agagaaagagaaatctgagc | tgg |
| 25310374 | −1 | gaagtcatcacacagtgaga | ggg |
| 25310375 | −1 | agaagtcatcacacagtgag | agg |
| 25310398 | −1 | cttcttgctgcatcatgaca | tgg |
| 25310410 | 1 | catgtcatgatgcagcaaga | agg |
| 25310422 | −1 | atggtgccaccatctggtga | ggg |
| 25310423 | −1 | catggtgccaccatctggtg | agg |
| 25310424 | 1 | gcaagaaggccctcaccaga | tgg |
| 25310427 | 1 | agaaggccctcaccagatgg | tgg |
| 25310428 | −1 | aaaagcatggtgccaccatc | tgg |
| 25310441 | 1 | agatggtggcaccatgcttt | tgg |
| 25310441 | −1 | ggctgggaagtccaaaagca | tgg |
| 25310457 | −1 | agctcacagttctagaggct | ggg |
| 25310458 | −1 | tagctcacagttctagaggc | tgg |
| 25310462 | −1 | gatttagctcacagttctag | agg |
| 25310506 | −1 | ctatgacaaaatatcaaact | ggg |
| 25310507 | −1 | gctatgacaaaatatcaaac | tgg |
| 25310530 | 1 | tttgtcatagcaacagaata | tgg |
| 25310592 | −1 | aaagccacttccacattttc | agg |
| 25310593 | 1 | gtaacagattcctgaaaatg | tgg |
| 25310599 | 1 | gattcctgaaaatgtggaag | tgg |
| 25310605 | 1 | tgaaaatgtggaagtggctt | tgg |
| 25310611 | 1 | tgtggaagtggctttggaac | tgg |
| 25310612 | 1 | gtggaagtggctttggaact | ggg |
| 25310618 | 1 | gtggctttggaactgggtga | tgg |
| 25310619 | 1 | tggctttggaactgggtgat | ggg |
| 25310625 | 1 | tggaactgggtgatgggaat | agg |
| 25310629 | 1 | actgggtgatgggaataggt | tgg |
| 25310642 | 1 | aataggttggaagagttttg | agg |
| 25310648 | 1 | ttggaagagttttgaggagc | agg |
| 25310670 | −1 | tgctccattcttgacaatac | agg |
| 25310677 | 1 | aaagcctgtattgtcaagaa | tgg |
| 25310691 | 1 | caagaatggagcattatgcc | agg |
| 25310696 | 1 | atggagcattatgccaggca | cgg |
| 25310698 | −1 | taagcctgagacaccgtgcc | tgg |
| 25310705 | 1 | tatgccaggcacggtgtctc | agg |
| 25310725 | −1 | ctttggcctcccaaagtgct | ggg |
| 25310726 | 1 | ggcttataatcccagcactt | tgg |
| 25310726 | −1 | gctttggcctcccaaagtgc | tgg |
| 25310727 | 1 | gcttataatcccagcacttt | ggg |
| 25310730 | 1 | tataatcccagcactttggg | agg |
| 25310740 | 1 | gcactttgggaggccaaagc | agg |
| 25310742 | −1 | ctcaggtgatccacctgctt | tgg |
| 25310743 | 1 | ctttgggaggccaaagcagg | tgg |
| 25310754 | 1 | caaagcaggtggatcacctg | agg |
| 25310759 | 1 | caggtggatcacctgaggtc | agg |
| 25310759 | −1 | ggtctcgaactcctgacctc | agg |
| 25310780 | −1 | tttcaccatgttagctaggc | tgg |
| 25310784 | −1 | agcgtttcaccatgttagct | agg |
| 25310786 | 1 | cgagaccagcctagctaaca | tgg |
| 25310814 | −1 | cagctaattttttgtatttt | tgg |
| 25310826 | 1 | caaaaatacaaaaaattagc | tgg |
| 25310827 | 1 | aaaaatacaaaaaattagct | ggg |
| 25310832 | 1 | tacaaaaaattagctgggcg | tgg |
| 25310835 | 1 | aaaaaattagctgggcgtgg | tgg |
| 25310853 | −1 | tcctgagtagctgagattac | agg |
| 25310863 | 1 | acctgtaatctcagctactc | agg |
| 25310866 | 1 | tgtaatctcagctactcagg | agg |
| 25310876 | 1 | gctactcaggaggctgaagc | agg |
| 25310895 | 1 | caggagaatcacttgaaccc | agg |
| 25310898 | 1 | gagaatcacttgaacccagg | agg |
| 25310901 | −1 | cactgcaacctctgcctcct | ggg |
| 25310902 | −1 | tcactgcaacctctgcctcc | tgg |
| 25310904 | 1 | cacttgaacccaggaggcag | agg |
| 25310944 | 1 | cgtgctattgcactccagct | tgg |
| 25310945 | 1 | gtgctattgcactccagctt | ggg |
| 25310947 | −1 | tttgctcttgttgcccaagc | tgg |
| 25310973 | 1 | ctttttttttttttttgaga | tgg |
| 25311027 | 1 | taaagacagttctgcagttc | tgg |
| 25311032 | 1 | acagttctgcagttctggtg | agg |
| 25311033 | 1 | cagttctgcagttctggtga | ggg |
| 25311041 | 1 | cagttctggtgagggcttaa | agg |
| 25311057 | −1 | ccagactttccctagttctg | ggg |
| 25311058 | 1 | taaaggaagaccccagaact | agg |
| 25311058 | −1 | tccagactttccctagttct | ggg |
| 25311059 | 1 | aaaggaagaccccagaacta | ggg |
| 25311059 | −1 | ttccagactttccctagttc | tgg |
| 25311068 | 1 | ccccagaactagggaaagtc | tgg |
| 25311081 | 1 | gaaagtctggaacttcttaa | tgg |
| 25311122 | 1 | tcagagtgctgatagaaata | tgg |
| 25311126 | 1 | agtgctgatagaaatatggc | tgg |
| 25311132 | 1 | gatagaaatatggctggtaa | agg |
| 25311144 | −1 | tatctgagacctcatcagaa | tgg |
| 25311146 | 1 | tggtaaaggccattctgatg | agg |
| 25311177 | 1 | agaactgaagaaccacgtgt | tgg |
| 25311178 | −1 | ttgctccagtttccaacacg | tgg |
| 25311184 | 1 | aagaaccacgtgttggaaac | tgg |
| 25311192 | 1 | cgtgttggaaactggagcaa | agg |
| 25311208 | −1 | atctttgcttctttataaaa | agg |
| 25311252 | −1 | ctgccttccataaatgactc | tgg |
| 25311256 | 1 | ttctgtgccagagtcattta | tgg |
| 25311260 | 1 | gtgccagagtcatttatgga | agg |
| 25311275 | 1 | atggaaggcagaaaatctgt | agg |
| 25311291 | 1 | ctgtaggtcagccatgttgt | agg |
| 25311291 | −1 | ttctttcattccctacaaca | tgg |
| 25311292 | 1 | tgtaggtcagccatgttgta | ggg |
| 25311352 | −1 | Gtactagttttcttatcagt | cgg |
| 25311379 | 1 | ctagtaCACATaaattagcc | agg |
| 25311384 | 1 | aCACATaaattagccaggcg | tgg |
| 25311386 | −1 | caggcgcccaccaccacgcc | tgg |
| 25311387 | 1 | CATaaattagccaggcgtgg | tgg |
| 25311390 | 1 | aaattagccaggcgtggtgg | tgg |
| 25311391 | 1 | aattagccaggcgtggtggt | ggg |
| 25311405 | −1 | tcccaggtagctgggaatac | agg |
| 25311413 | −1 | cctcagcctcccaggtagct | ggg |
| 25311414 | 1 | cgcctgtattcccagctacc | tgg |
| 25311414 | −1 | gcctcagcctoccaggtagc | tgg |
| 25311415 | 1 | gcctgtattcccagctacct | ggg |
| 25311418 | 1 | tgtattcccagctacctggg | agg |
| 25311421 | −1 | ttctcctgcctcagcctccc | agg |
| 25311424 | 1 | cccagctacctgggaggctg | agg |
| 25311428 | 1 | gctacctgggaggctgaggc | agg |
| 25311435 | 1 | gggaggctgaggcaggagaa | tgg |
| 25311446 | 1 | gcaggagaatggcatgaacc | cgg |
| 25311447 | 1 | caggagaatggcatgaaccc | ggg |
| 25311450 | 1 | gagaatggcatgaacccggg | agg |
| 25311453 | −1 | cactgcaagctctgcctccc | ggg |
| 25311454 | −1 | tcactgcaagctctgcctcc | cgg |
| 25311478 | −1 | ggagtgcagtggcgcgatct | tgg |
| 25311489 | −1 | tcgcccaggctggagtgcag | tgg |
| 25311496 | 1 | cgcgccactgcactccagcc | tgg |
| 25311497 | 1 | gcgccactgcactccagcct | ggg |
| 25311499 | −1 | ttttgctctgtcgcccaggc | tgg |
| 25311503 | −1 | ggagttttgctctgtcgccc | agg |
| 25311524 | −1 | ttttttttttctttttgaga | cgg |
| 25311537 | 1 | gtctcaaaaagaaaaaaaaa | agg |
| 25311575 | 1 | tacacatagaacaaagccag | agg |
| 25311580 | −1 | ttgtcctgatgaacagcctc | tgg |
| 25311587 | 1 | aaagccagaggctgttcatc | agg |
| 25311593 | 1 | agaggctgttcatcaggaca | agg |
| 25311594 | 1 | gaggctgttcatcaggacaa | ggg |
| 25311615 | −1 | gaagatctctgaaatggctt | tgg |
| 25311621 | −1 | agtcttgaagatctctgaaa | tgg |
| 25311645 | −1 | ctctgggccagtaatgggag | ggg |
| 25311646 | −1 | gctctgggccagtaatggga | ggg |
| 25311647 | −1 | agctctgggccagtaatggg | agg |
| 25311649 | 1 | aagactgcccctcccattac | tgg |
| 25311650 | −1 | tagagctctgggccagtaat | ggg |
| 25311651 | −1 | ttagagctctgggccagtaa | tgg |
| 25311661 | −1 | ttctgccctcttagagctct | ggg |
| 25311662 | −1 | attctgccctcttagagctc | tgg |
| 25311666 | 1 | tactggcccagagctctaag | agg |
| 25311667 | 1 | actggcccagagctctaaga | ggg |
| 25311675 | 1 | agagctctaagagggcagaa | tgg |
| 25311680 | 1 | tctaagagggcagaatggtt | tgg |
| 25311697 | −1 | aggcagccctgggcagcagc | tgg |
| 25311701 | 1 | ggaatgaccagctgctgccc | agg |
| 25311702 | 1 | gaatgaccagctgctgccca | ggg |
| 25311707 | −1 | cagagacccaaggcagccct | ggg |
| 25311708 | −1 | gcagagacccaaggcagccc | tgg |
| 25311711 | 1 | gctgctgcccagggctgcct | tgg |
| 25311712 | 1 | ctgctgcccagggctgcctt | ggg |
| 25311717 | −1 | atgtggggagcagagaccca | agg |
| 25311732 | −1 | aatgctgcaccagaaatgtg | ggg |
| 25311733 | −1 | gaatgctgcaccagaaatgt | ggg |
| 25311734 | 1 | gtctctgctccccacatttc | tgg |
| 25311734 | −1 | ggaatgctgcaccagaaatg | tgg |
| 25311755 | −1 | aaccacagctgggatggctg | agg |
| 25311761 | −1 | cacctgaaccacagctggga | tgg |
| 25311764 | 1 | ttcctcagccatcccagctg | tgg |
| 25311765 | −1 | tggccacctgaaccacagct | ggg |
| 25311766 | −1 | gtggccacctgaaccacagc | tgg |
| 25311770 | 1 | agccatcccagctgtggttc | agg |
| 25311773 | 1 | catcccagctgtggttcagg | tgg |
| 25311780 | 1 | gctgtggttcaggtggccac | agg |
| 25311785 | −1 | taccttccacatcacacctg | tgg |
| 25311790 | 1 | aggggccacaggtgtgatg | tgg |
| 25311794 | 1 | ggccacaggtgtgatgtgga | agg |
| 25311813 | 1 | aaggtaaaagtcataaacct | tgg |
| 25311819 | −1 | gtgccatgtgtatgctgcca | agg |
| 25311827 | 1 | aaaccttggcagcatacaca | tgg |
| 25311842 | 1 | acacatggcactaattttgc | agg |
| 25311865 | 1 | tgtgcagaatgcaaaagctg | agg |
| 25311866 | 1 | gtgcagaatgcaaaagctga | ggg |
| 25311867 | 1 | tgcagaatgcaaaagctgag | ggg |
| 25311868 | 1 | gcagaatgcaaaagctgagg | ggg |
| 25311884 | −1 | tttgaaatgtaggtggaaga | agg |
| 25311891 | −1 | agcaccctttgaaatgtagg | tgg |
| 25311894 | −1 | cacagcaccctttgaaatgt | agg |
| 25311897 | 1 | ttcttccacctacatttcaa | agg |
| 25311898 | 1 | tcttccacctacatttcaaa | ggg |
| 25311922 | −1 | ctactaggggctctctgggg | tgg |
| 25311925 | −1 | gctctactaggggctctctg | ggg |
| 25311926 | −1 | tgctctactaggggctctct | ggg |
| 25311927 | −1 | ctgctctactaggggctctc | tgg |
| 25311935 | −1 | actagaccctgctctactag | ggg |
| 25311936 | −1 | cactagaccctgctctacta | ggg |
| 25311937 | −1 | ccactagaccctgctctact | agg |
| 25311939 | 1 | cagagagcccctagtagagc | agg |
| 25311940 | 1 | agagagcccctagtagagca | ggg |
| 25311948 | 1 | cctagtagagcagggtctag | tgg |
| 25311958 | 1 | cagggtctagtggagctaca | agg |
| 25311959 | 1 | agggtctagtggagctacaa | ggg |
| 25311962 | 1 | gtctagtggagctacaaggg | tgg |
| 25311963 | 1 | tctagtggagctacaagggt | ggg |
| 25311964 | 1 | ctagtggagctacaagggtg | ggg |
| 25311976 | −1 | ccattctggggtcttggcgg | tgg |
| 25311979 | −1 | ctaccattctggggtcttgg | cgg |
| 25311982 | −1 | gctctaccattctggggtct | tgg |
| 25311987 | 1 | ccaccgccaagaccccagaa | tgg |
| 25311988 | −1 | atgatagctctaccattctg | ggg |
| 25311989 | −1 | tatgatagctctaccattct | ggg |
| 25311990 | −1 | ctatgatagctctaccattc | tgg |
| 25312017 | 1 | atcatagtgcaatgccagct | tgg |
| 25312018 | 1 | tcatagtgcaatgccagctt | ggg |
| 25312020 | −1 | tgcctgcagttctcccaagc | tgg |
| 25312029 | 1 | tgccagcttgggagaactgc | agg |
| 25312050 | −1 | atgttgcacttcgcacaggt | tgg |
| 25312054 | −1 | gcccatgttgcacttcgcac | agg |
| 25312063 | 1 | aacctgtgcgaagtgcaaca | tgg |
| 25312064 | 1 | acctgtgcgaagtgcaacat | ggg |
| 25312081 | −1 | tctgcccctgtggttttgct | ggg |
| 25312082 | −1 | ctctgcccctgtggttttgc | tgg |
| 25312086 | 1 | gcagaacccagcaaaaccac | agg |
| 25312087 | 1 | cagaacccagcaaaaccaca | ggg |
| 25312088 | 1 | agaacccagcaaaaccacag | ggg |
| 25312091 | −1 | ttcggggagctctgcccctg | tgg |
| 25312107 | −1 | tttggacccccgaagcttcg | ggg |
| 25312108 | −1 | atttggacccccgaagcttc | ggg |
| 25312109 | 1 | ggcagagctccccgaagctt | cgg |
| 25312109 | −1 | aatttggacccccgaagctt | cgg |
| 25312110 | 1 | gcagagctccccgaagcttc | ggg |
| 25312111 | 1 | cagagctccccgaagcttcg | ggg |
| 25312112 | 1 | agagctccccgaagcttcgg | ggg |
| 25312125 | −1 | cctggacacactatggaatt | tgg |
| 25312132 | −1 | gccacctcctggacacacta | tgg |
| 25312136 | 1 | ccaaattccatagtgtgtcc | agg |
| 25312139 | 1 | aattccatagtgtgtccagg | agg |
| 25312142 | 1 | tccatagtgtgtccaggagg | tgg |
| 25312143 | −1 | ttactctgtgtgccacctcc | tgg |
| 25312170 | 1 | agagtaaaagatcattctga | agg |
| 25312177 | 1 | aagatcattctgaaggttta | agg |
| 25312200 | 1 | tttaatgttgttttctatgt | tgg |
| 25312201 | 1 | ttaatgttgttttctatgtt | ggg |
| 25312217 | 1 | tgttgggttttgtactttcc | tgg |
| 25312224 | −1 | gaaaaagggtaactggttcc | agg |
| 25312231 | −1 | ggcaagggaaaaagggtaac | tgg |
| 25312238 | −1 | aaaaagaggcaagggaaaaa | ggg |
| 25312239 | −1 | gaaaaagaggcaagggaaaa | agg |
| 25312246 | −1 | ctaaaaggaaaaagaggcaa | ggg |
| 25312247 | −1 | tctaaaaggaaaaagaggca | agg |
| 25312252 | −1 | cccattctaaaaggaaaaag | agg |
| 25312261 | −1 | acagacattcccattctaaa | agg |
| 25312262 | 1 | gcctctttttccttttagaa | tgg |
| 25312263 | 1 | cctctttttccttttagaat | ggg |
| 25312284 | −1 | tacaacagtggaacaggcat | agg |
| 25312290 | −1 | ccaaaatacaacagtggaac | agg |
| 25312296 | −1 | tgacttccaaaatacaacag | tgs |
| 25312301 | 1 | cctgttccactgttgtattt | tgg |
| 25312330 | 1 | ataacttgttttgactttac | agg |
| 25312344 | 1 | ctttacaggcttacagccag | agg |
| 25312345 | 1 | tttacaggcttacagccaga | ggg |
| 25312349 | −1 | attctatgggagattccctc | tgg |
| 25312362 | −1 | taaggtacaattcattctat | ggg |
| 25312363 | −1 | ttaaggtacaattcattcta | tgg |
| 25312414 | −1 | actcaaaattccaaagtcca | tgg |
| 25312415 | 1 | ttagatgagaccatggactt | tgg |
| 25312428 | 1 | tggactttggaattttgagt | tgg |
| 25312434 | 1 | ttggaattttgagttggtgc | tgg |
| 25312452 | 1 | gctggaacaagttaagactt | tgg |
| 25312453 | 1 | ctggaacaagttaagacttt | ggg |
| 25312454 | 1 | tggaacaagttaagactttg | ggg |
| 25312455 | 1 | ggaacaagttaagactttgg | ggg |
| 25312469 | 1 | ctttgggggttgtctaagtg | tgg |
| 25312490 | −1 | tcccaaatcactgggattac | agg |
| 25312498 | −1 | cctcaacctcccaaatcact | ggg |
| 25312499 | 1 | tgcctgtaatcccagtgatt | tgg |
| 25312499 | −1 | acctcaacctcccaaatcac | tgg |
| 25312500 | 1 | gcctgtaatcccagtgattt | ggg |
| 25312503 | 1 | tgtaatcccagtgatttggg | agg |
| 25312509 | 1 | cccagtgatttgggaggttg | agg |
| 25312512 | 1 | agtgatttgggaggttgagg | tgg |
| 25312513 | 1 | gtgatttgggaggttgaggt | ggg |
| 25312516 | 1 | atttgggaggttgaggtggg | agg |
| 25312532 | 1 | tgggaggattgcttgagccc | agg |
| 25312538 | −1 | caggctggtcttgagctcct | ggg |
| 25312539 | −1 | ccaggctggtcttgagctcc | tgg |
| 25312550 | 1 | ccaggagctcaagaccagcc | tgg |
| 25312551 | 1 | caggagctcaagaccagcct | ggg |
| 25312553 | −1 | tctcactatgttgcccaggc | tgg |
| 25312557 | −1 | caggtctcactatgttgccc | agg |
| 25312576 | −1 | tttttattttttgtagagac | agg |
| 25312604 | 1 | taaaaataaaaaaattagcc | agg |
| 25312611 | −1 | caggtatatgccacaatacc | tgg |
| 25312612 | 1 | aaaaaattagccaggtattg | tgg |
| 25312630 | −1 | tcctgagtagctagaattac | agg |
| 25312640 | 1 | acctgtaattctagctactc | agg |
| 25312643 | 1 | tgtaattctagctactcagg | agg |
| 25312649 | 1 | tctagctactcaggaggctg | agg |
| 25312656 | 1 | actcaggaggctgaggtgag | agg |
| 25312672 | 1 | tgagaggatcacttgagccc | agg |
| 25312678 | −1 | cactgcagcctcaaactcct | ggg |
| 25312679 | −1 | tcactgcagcctcaaactcc | tgg |
| 25312681 | 1 | cacttgagcccaggagtttg | agg |
| 25312697 | 1 | tttgaggctgcagtgagcta | tgg |
| 25312714 | −1 | ttgccctggctggaatgcag | tgg |
| 25312721 | 1 | cgtgccactgcattccagcc | agg |
| 25312722 | 1 | gtgccactgcattccagcca | ggg |
| 25312724 | −1 | tctcactctgttgccctggc | tgg |
| 25312728 | −1 | agagtctcactctgttgccc | tgg |
| 25312773 | 1 | taaaattaaataaacttagc | tgg |
| 25312779 | 1 | taaataaacttagctggata | tgg |
| 25312782 | 1 | ataaacttagctggatatgg | tgg |
| 25312808 | −1 | tctcagcctcctgagtagct | agg |
| 25312810 | 1 | atctgtagtcctagctactc | agg |
| 25312813 | 1 | tgtagtcctagctactcagg | agg |
| 25312823 | 1 | gctactcaggaggctgagac | agg |
| 25312826 | 1 | actcaggaggctgagacagg | agg |
| 25312842 | 1 | caggaggattacttgagcca | agg |
| 25312848 | −1 | cactgcagcctcaaactect | tgg |
| 25312851 | 1 | tacttgagccaaggagtttg | agg |
| 25312884 | −1 | tcatccaggctggaatgcag | tgg |
| 25312891 | 1 | catgccactgcattccagcc | tgg |
| 25312894 | −1 | ttttgctctatcatccaggc | tgg |
| 25312898 | −1 | gggattttgctctatcatcc | agg |
| 25312918 | −1 | ttttttttttttttagagat | ggg |
| 25312919 | −1 | tttttttttttttttagaga | tgg |
| 25312965 | 1 | aaaaaaaactttagtgctat | tgg |
| 25312988 | 1 | aatgaattttgcatgtaaga | agg |
| 25313001 | 1 | tgtaagaaggacatgcattt | tgg |
| 25313002 | 1 | gtaagaaggacatgcatttt | ggg |
| 25313003 | 1 | taagaaggacatgcattttg | ggg |
| 25313004 | 1 | aagaaggacatgcattttgg | ggg |
| 25313008 | 1 | aggacatgcattttgggggc | tgg |
| 25313009 | 1 | ggacatgcattttgggggct | ggg |
| 25313010 | 1 | gacatgcattttgggggctg | ggg |
| 25313014 | 1 | tgcattttgggggctggggc | agg |
| 25313023 | 1 | ggggctggggcaggatgctg | tgg |
| 25313046 | −1 | ttccaacacatgaaatttga | ggg |
| 25313047 | −1 | tttccaacacatgaaatttg | agg |
| 25313055 | 1 | atccctcaaatttcatgtgt | tgg |
| 25313078 | −1 | atttcatcaacatatgaatt | tgg |
| 25313092 | 1 | aattcatatgttgatgaaat | tgg |
| 25313095 | 1 | tcatatgttgatgaaattgg | agg |
| 25313107 | 1 | gaaattggaggtgaagcctt | tgg |
| 25313108 | 1 | aaattggaggtgaagccttt | ggg |
| 25313111 | 1 | ttggaggtgaagcctttggg | agg |
| 25313112 | −1 | taatcctagttacctcccaa | agg |
| 25313119 | 1 | gaagcctttgggaggtaact | agg |
| 25313138 | 1 | taggattagataaagtcatc | agg |
| 25313139 | 1 | aggattagataaagtcatca | ggg |
| 25313142 | 1 | attagataaagtcatcaggg | tgg |
| 25313143 | 1 | ttagataaagtcatcagggt | ggg |
| 25313144 | 1 | tagataaagtcatcagggtg | ggg |
| 25313156 | −1 | agccaccagtctcatcatag | ggg |
| 25313157 | −1 | aagccaccagtctcatcata | ggg |
| 25313158 | −1 | taagccaccagtctcatcat | agg |
| 25313162 | 1 | tggggcccctatgatgagac | tgg |
| 25313165 | 1 | ggcccctatgatgagactgg | tgg |
| 25313176 | 1 | tgagactggtggcttacaag | agg |
| 25313216 | −1 | gagggtatcacatggcaaga | ggg |
| 25313217 | −1 | agagggtatcacatggcaag | agg |
| 25313224 | −1 | acatggcagagggtatcaca | tgg |
| 25313234 | −1 | gcctgccattacatggcaga | ggg |
| 25313235 | −1 | tgcctgccattacatggcag | agg |
| 25313240 | 1 | tgataccctctgccatgtaa | tgg |
| 25313241 | −1 | ttgctgtgcctgccattaca | tgg |
| 25313244 | 1 | accctctgccatgtaatggc | agg |
| 25313257 | 1 | taatggcaggcacagcaaga | agg |
| 25313270 | −1 | catgctgctggcatctgttg | agg |
| 25313282 | −1 | gaagtccaagaacatgctgc | tgg |
| 25313288 | 1 | agatgccagcagcatgttct | tgg |
| 25313304 | −1 | agctcatggttctggaggct | ggg |
| 25313305 | −1 | tagctcatggttctggaggc | tgg |
| 25313309 | −1 | tatatagctcatggttctgg | agg |
| 25313312 | −1 | gtatatatagctcatggttc | tgg |
| 25313318 | −1 | aaataagtatatatagctca | tgg |
| 25313350 | 1 | tttacaaattacccattctg | tgg |
| 25313350 | −1 | ataacagaataccacagaat | ggg |
| 25313351 | −1 | tataacagaataccacagaa | tgg |
| 25313395 | 1 | atgaactgagataatataca | tgg |
| 25313465 | 1 | tgtagttgtgagattcatcc | agg |
| 25313472 | −1 | tacagcaatgcttaacaacc | tgg |
| 25313495 | −1 | actatatcccagtggaaaaa | ggg |
| 25313496 | −1 | cactatatcccagtggaaaa | agg |
| 25313498 | 1 | ttgctgtaccctttttccac | tgg |
| 25313499 | 1 | tgctgtaccctttttccact | ggg |
| 25313503 | −1 | gacagaacactatatcccag | tgg |
| 25313522 | 1 | atatagtgttctgtcatgCT | TGG |
| 25313523 | 1 | tatagtgttctgtcatgCTT | GGG |
| 25313539 | 1 | gCTTGGGTCTTAATTTATAA | AGG |
| 25313550 | 1 | AATTTATAAAGGTGACTGAG | TGG |
| 25313571 | −1 | aactttccttccaatAATAC | TGG |
| 25313572 | 1 | GCATTTTCTTCCAGTATTat | tgg |
| 25313576 | 1 | TTTCTTCCAGTATTattgga | agg |
| 25313609 | −1 | gttctgcctcttgtttacag | ggg |
| 25313610 | −1 | tgttctgcctcttgtttaca | ggg |
| 25313611 | −1 | gtgttctgcctcttgtttac | agg |
| 25313614 | 1 | acagttcccctgtaaacaag | agg |
| 25313632 | 1 | agaggcagaacacgtcatgc | agg |
| 25313633 | 1 | gaggcagaacacgtcatgca | ggg |
| 25313645 | −1 | ctggatgatacagttttgtg | tgg |
| 25313657 | 1 | cacacaaaactgtatcatcc | agg |
| 25313658 | 1 | acacaaaactgtatcatcca | ggg |
| 25313664 | 1 | aactgtatcatccagggacc | agg |
| 25313664 | −1 | tctttctgctgcctggtccc | tgg |
| 25313671 | −1 | ccccctctctttctgctgcc | tgg |
| 25313679 | 1 | ggaccaggcagcagaaagag | agg |
| 25313680 | 1 | gaccaggcagcagaaagaga | ggg |
| 25313681 | 1 | accaggcagcagaaagagag | ggg |
| 25313682 | 1 | ccaggcagcagaaagagagg | ggg |
| 25313688 | 1 | agcagaaagagagggggaac | tgg |
| 25313689 | 1 | gcagaaagagagggggaact | ggg |
| 25313707 | −1 | CCCACCACTCTTTTTCataa | agg |
| 25313714 | 1 | tatgcctttatGAAAAAGAG | TGG |
| 25313717 | 1 | gcctttatGAAAAAGAGTGG | TGG |
| 25313718 | 1 | cctttatGAAAAAGAGTGGT | GGG |
| 25313729 | 1 | AAGAGTGGTGGGAGAGTAAC | TGG |
| 25313730 | 1 | AGAGTGGTGGGAGAGTAACT | GGG |
| 25313735 | 1 | GGTGGGAGAGTAACTGGGTG | AGG |
| 25313736 | 1 | GTGGGAGAGTAACTGGGTGA | GGG |
| 25313749 | 1 | TGGGTGAGGGCATCCACTAA | TGG |
| 25313750 | 1 | GGGTGAGGGCATCCACTAAT | GGG |
| 25313751 | −1 | TTTCACTTCCTGCCCATTAG | TGG |
| 25313754 | 1 | GAGGGCATCCACTAATGGGC | AGG |
| 25313790 | 1 | TATGTTAGAATTTGTAGCTG | AGG |
| 25313791 | 1 | ATGTTAGAATTTGTAGCTGA | GGG |
| 25313792 | 1 | TGTTAGAATTTGTAGCTGAG | GGG |
| 25313820 | −1 | AAGTCAGCTTTCTCAGGCAT | AGG |
| 25313826 | −1 | TCTTGCAAGTCAGCTTTCTC | AGG |
| 25313858 | 1 | GAAAATGAGATAAACAACTT | TGG |
| 25313870 | 1 | AACAACTTTGGCCATTAGTG | tgg |
| 25313870 | −1 | ttatgacagggccaCACTAA | TGG |
| 25313882 | −1 | tctggcattcatttatgaca | ggg |
| 25313883 | −1 | atctggcattcatttatgac | agg |
| 25313897 | 1 | gtcataaatgaatgccagat | agg |
| 25313900 | −1 | agattctctatttgcctatc | tgg |
| 25313927 | 1 | agaatctaagaaaaGATAGT | TGG |
| 25313949 | −1 | attctgctgcattcacacaa | tgg |
| 25313980 | 1 | aatttatttatccattattg | agg |
| 25313980 | −1 | acccaaatcctcctcaataa | tgg |
| 25313983 | 1 | ttatttatccattattgagg | agg |
| 25313989 | 1 | atccattattgaggaggatt | tgg |
| 25313990 | 1 | tccattattgaggaggattt | ggg |
| 25314005 | 1 | gatttgggtagtttccagtt | tgg |
| 25314008 | −1 | tattcataatagctccaaac | tgg |
| 25314044 | −1 | aaaagtgctagaatgttcat | agg |
| 25314063 | 1 | cattctagcacttttatttt | tgg |
| 25314106 | −1 | aattcaacaatttcacttct | agg |
| 25314147 | 1 | attcacacagtcagctttag | tgg |
| 25314192 | −1 | tacactactggtgattagat | tgg |
| 25314204 | −1 | aaggagcttctatacactac | tgg |
| 25314223 | −1 | ttggcaaaatgtggagtaaa | agg |
| 25314232 | −1 | caccaagtgttggcaaaatg | tgg |
| 25314241 | 1 | ctccacattttgccaacact | tgg |
| 25314242 | −1 | agaaggaaaacaccaagtgt | tgg |
| 25314259 | −1 | taaatgactaatcaaaaaga | agg |
| 25314291 | −1 | gatatcaaaatgtaaacaat | agg |
| 25314315 | −1 | tgctccatttagttagttat | tgg |
| 25314322 | 1 | atctccaataactaactaaa | tgg |
| 25314345 | 1 | agcacttttaatatgctttt | tgg |
| 25314403 | −1 | agaacaccacaatagaaaat | ggg |
| 25314404 | −1 | cagaacaccacaatagaaaa | tgg |
| 25314408 | 1 | agtttgcccattttctattg | tgg |
| 25314438 | 1 | tctttttcttattgatttgt | agg |
| 25314454 | −1 | attcatatccaggatacgta | agg |
| 25314457 | 1 | taggaattccttacgtatcc | tgg |
| 25314464 | −1 | acaaagtgggattcatatcc | agg |
| 25314477 | −1 | aaaaaggtaacgcacaaagt | ggg |
| 25314478 | −1 | gaaaaaggtaacgcacaaag | tgg |
| 25314493 | −1 | aaagaaagaaagaaggaaaa | agg |
| 25314500 | −1 | gtttcaaaaagaaagaaaga | agg |
| 25314530 | −1 | attccagcctgggtgacaga | agg |
| 25314534 | 1 | agagtctccttctgtcaccc | agg |
| 25314538 | 1 | tctccttctgtcacccaggc | tgg |
| 25314540 | −1 | gcgccactgcattccagcct | ggg |
| 25314541 | −1 | agcgccactgcattccagcc | tgg |
| 25314548 | 1 | tcacccaggctggaatgcag | tgg |
| 25314571 | −1 | tgggaggcagaggttgtagt | ggg |
| 25314572 | −1 | ctgggaggcagaggttgtag | tgg |
| 25314581 | −1 | tgcttgaagctgggaggcag | agg |
| 25314587 | −1 | gagaattgcttgaagctggg | agg |
| 25314590 | −1 | tatgagaattgcttgaagct | ggg |
| 25314591 | −1 | gtatgagaattgcttgaagc | tgg |
| 25314619 | −1 | tgtaatctaagctactcagg | agg |
| 25314622 | −1 | gcctgtaatctaagctactc | agg |
| 25314632 | 1 | tcctgagtagcttagattac | agg |
| 25314650 | −1 | cagaagttagctgggcatgg | tgg |
| 25314653 | −1 | atacagaagttagctgggca | tgg |
| 25314658 | −1 | tgtctatacagaagttagct | ggg |
| 25314659 | −1 | ttgtctatacagaagttagc | tgg |
| 25314682 | 1 | tgtatagacaaaataatttt | tgg |
| 25314692 | 1 | aaataatttttggtagagac | agg |
| 25314693 | 1 | aataatttttggtagagaca | ggg |
| 25314707 | 1 | gagacagggttttgccatgt | tgg |
| 25314710 | −1 | caagatcagcctgtccaaca | tgg |
| 25314712 | 1 | agggttttgccatgttggac | agg |
| 25314722 | 1 | catgttggacaggctgatct | tgg |
| 25314730 | 1 | acaggctgatcttggactcc | tgg |
| 25314737 | −1 | ggtgggccaaagttgaggcc | agg |
| 25314742 | 1 | tggactcctggcctcaactt | tgg |
| 25314742 | −1 | gccaaggtgggccaaagttg | agg |
| 25314752 | 1 | gcctcaactttggcccacct | tgg |
| 25314754 | −1 | gcactttgggaggccaaggt | ggg |
| 25314755 | −1 | ggcactttgggaggccaagg | tgg |
| 25314758 | −1 | cctggcactttgggaggcca | agg |
| 25314764 | −1 | tgtaatcctggcactttggg | agg |
| 25314767 | −1 | acctgtaatcctggcacttt | ggg |
| 25314768 | −1 | cacctgtaatcctggcactt | tgg |
| 25314769 | 1 | ccttggcctcccaaagtgcc | agg |
| 25314776 | −1 | gtggctcacacctgtaatcc | tgg |
| 25314777 | 1 | tcccaaagtgccaggattac | agg |
| 25314795 | −1 | aaaaggtgggctgggcatgg | tgg |
| 25314798 | −1 | agtaaaaggtgggctgggca | tgg |
| 25314803 | −1 | aagaaagtaaaaggtgggct | ggg |
| 25314804 | −1 | taagaaagtaaaaggtgggc | tgg |
| 25314808 | −1 | ccattaagaaagtaaaaggt | ggg |
| 25314809 | −1 | accattaagaaagtaaaagg | tgg |
| 25314812 | −1 | gacaccattaagaaagtaaa | agg |
| 25314819 | 1 | cccaccttttactttcttaa | tgg |
| 25314839 | 1 | tggtgtcttttgaacaagag | agg |
| 25314869 | −1 | aaagggaacaatgataaatt | ggg |
| 25314870 | −1 | taaagggaacaatgataaat | tgg |
| 25314886 | −1 | ataaaagaactaaacataaa | ggg |
| 25314887 | −1 | cataaaagaactaaacataa | agg |
| 25314911 | −1 | GGCTgcaaaaattcttaaaa | agg |
| 25314929 | 1 | aagaatttttgcAGCCAgcg | cgg |
| 25314932 | 1 | aatttttgcAGCCAgcgcgg | tgg |
| 25314932 | −1 | acaggtgtgagccaccgcgc | TGG |
| 25314950 | −1 | tcccaaagtgctgggattac | agg |
| 25314958 | −1 | cctcagcctcccaaagtgct | ggg |
| 25314959 | 1 | cacctgtaatcccagcactt | tgg |
| 25314959 | −1 | gcctcagcctcccaaagtgc | tgg |
| 25314960 | 1 | acctgtaatcccagcacttt | ggg |
| 25314963 | 1 | tgtaatcccagcactttggg | agg |
| 25314969 | 1 | cccagcactttgggaggctg | agg |
| 25314973 | 1 | gcactttgggaggctgaggc | tgg |
| 25314976 | 1 | ctttgggaggctgaggctgg | cgg |
| 25314985 | 1 | gctgaggctggcggatcaca | agg |
| 25315008 | 1 | tcaagagatcgagatcatcc | tgg |
| 25315015 | −1 | agggcttcaccatgttggcc | agg |
| 25315017 | 1 | cgagatcatcctggccaaca | tgg |
| 25315020 | −1 | ggcacagggcttcaccatgt | tgg |
| 25315034 | −1 | ttgtatttttagtaggcaca | ggg |
| 25315035 | −1 | tttgtatttttagtaggcac | agg |
| 25315041 | −1 | taattttttgtatttttagt | agg |
| 25315057 | 1 | taaaaatacaaaaaattagc | tgg |
| 25315058 | 1 | aaaaatacaaaaaattagct | ggg |
| 25315066 | 1 | aaaaaattagctgggcgttg | tgg |
| 25315084 | −1 | tcccgagtagctgagactac | agg |
| 25315093 | 1 | tgcctgtagtctcagctact | cgg |
| 25315094 | 1 | gcctgtagtctcagctactc | ggg |
| 25315097 | 1 | tgtagtctcagctactcggg | agg |
| 25315120 | −1 | gtcaccaggctggagtgcag | tgg |
| 25315127 | 1 | cacgccactgcactccagcc | tgg |
| 25315130 | −1 | gtcttgctgtgtcaccaggc | tgg |
| 25315134 | −1 | tggagtcttgctgtgtcacc | agg |
| 25315154 | −1 | aaaaaaatttttttttgaga | tgg |
| 25315172 | 1 | aaaaaaaaattttttttGCA | AGG |
| 25315196 | −1 | TTTTTAGGAAAAAAATCAGG | GGG |
| 25315197 | −1 | ATTTTTAGGAAAAAAATCAG | GGG |
| 25315198 | −1 | GATTTTTAGGAAAAAAATCA | GGG |
| 25315199 | −1 | TGATTTTTAGGAAAAAAATC | AGG |
| 25315211 | −1 | TCTAATAATAAGTGATTTTT | AGG |
| 25315280 | 1 | attcaacaaatatttccctg | agg |
| 25315284 | −1 | ttcaggttatcaaaacctca | ggg |
| 25315285 | −1 | gttcaggttatcaaaacctc | agg |
| 25315301 | −1 | cccagctccaaacacagttc | agg |
| 25315305 | 1 | ttgataacctgaactgtgtt | tgg |
| 25315311 | 1 | acctgaactgtgtttggagc | tgg |
| 25315312 | 1 | cctgaactgtgtttggagct | ggg |
| 25315313 | 1 | ctgaactgtgtttggagctg | ggg |
| 25315316 | 1 | aactgtgtttggagctgggg | agg |
| 25315346 | 1 | CTATTGAAGATATACAAAGA | TGG |
| 25315357 | 1 | ATACAAAGATGGCAAAGATG | AGG |
| 25315358 | 1 | TACAAAGATGGCAAAGATGA | GGG |
| 25315363 | 1 | AGATGGCAAAGATGAGGGCC | TGG |
| 25315370 | −1 | CCTTCCGTGTGGCAAGCTCC | AGG |
| 25315377 | 1 | AGGGCCTGGAGCTTGCCACA | CGG |
| 25315381 | 1 | CCTGGAGCTTGCCACACGGA | AGG |
| 25315381 | −1 | CAGCCATCCCCCCTTCCGTG | TGG |
| 25315382 | 1 | CTGGAGCTTGCCACACGGAA | GGG |
| 25315383 | 1 | TGGAGCTTGCCACACGGAAG | GGG |
| 25315384 | 1 | GGAGCTTGCCACACGGAAGG | GGG |
| 25315385 | 1 | GAGCTTGCCACACGGAAGGG | GGG |
| 25315389 | 1 | TTGCCACACGGAAGGGGGGA | TGG |
| 25315401 | 1 | AGGGGGGATGGCTGCCTGAA | TGG |
| 25315404 | −1 | AACTACCTGCCCAACCATTC | AGG |
| 25315405 | 1 | GGGATGGCTGCCTGAATGGT | TGG |
| 25315406 | 1 | GGATGGCTGCCTGAATGGTT | GGG |
| 25315410 | 1 | GGCTGCCTGAATGGTTGGGC | AGG |
| 25315442 | −1 | GCCACCCTGCTGCTCATGTA | GGG |
| 25315443 | −1 | TGCCACCCTGCTGCTCATGT | AGG |
| 25315448 | 1 | GCACTCCCTACATGAGCAGC | AGG |
| 25315449 | 1 | CACTCCCTACATGAGCAGCA | GGG |
| 25315452 | 1 | TCCCTACATGAGCAGCAGGG | TGG |
| 25315516 | 1 | ttctttttttttttttgaga | tgg |
| 25315537 | 1 | ggagtctcgctgtgttgccc | agg |
| 25315541 | 1 | tctcgctgtgttgcccaggc | tgg |
| 25315543 | −1 | acgccactgcactccagcct | ggg |
| 25315544 | −1 | cacgccactgcactccagcc | tgg |
| 25315551 | 1 | ttgcccaggctggagtgcag | tgg |
| 25315587 | 1 | cactgcaaactccacctccc | agg |
| 25315587 | −1 | aacggcgtgaacctgggagg | tgg |
| 25315590 | −1 | gagaacggcgtgaacctggg | agg |
| 25315593 | −1 | caggagaacggcgtgaacct | ggg |
| 25315594 | −1 | gcaggagaacggcgtgaacc | tgg |
| 25315605 | −1 | aggaggctgaggcaggagaa | cgg |
| 25315612 | −1 | gctactcaggaggctgaggc | agg |
| 25315616 | −1 | cccagctactcaggaggctg | agg |
| 25315622 | −1 | tgtagtcccagctactcagg | agg |
| 25315625 | −1 | gcctgtagtcccagctactc | agg |
| 25315626 | 1 | gcctcagcctcctgagtagc | tgg |
| 25315627 | 1 | cctcagcctcctgagtagct | ggg |
| 25315635 | 1 | tcctgagtagctgggactac | agg |
| 25315649 | −1 | cattagccgggagtggtggc | agg |
| 25315653 | −1 | aaaacattagccgggagtgg | tgg |
| 25315654 | 1 | caggcgcctgccaccactcc | cgg |
| 25315656 | −1 | tacaaaacattagccgggag | tgg |
| 25315661 | −1 | aaaaatacaaaacattagcc | ggg |
| 25315662 | −1 | taaaaatacaaaacattagc | cgg |
| 25315683 | 1 | ttttgtatttttagtagaga | agg |
| 25315684 | 1 | tttgtatttttagtagagaa | ggg |
| 25315685 | 1 | ttgtatttttagtagagaag | ggg |
| 25315704 | 1 | ggggtttcactgtgttagcc | agg |
| 25315708 | 1 | tttcactgtgttagccagga | tgg |
| 25315711 | −1 | tcaggagatggagaccatcc | tgg |
| 25315723 | −1 | cagatcatgaggtcaggaga | tgg |
| 25315729 | −1 | ggcgggcagatcatgaggtc | agg |
| 25315734 | −1 | gccgaggcgggcagatcatg | agg |
| 25315744 | 1 | acctcatgatctgcccgcct | cgg |
| 25315746 | −1 | acactttgggaggccgaggc | ggg |
| 25315747 | −1 | cacactttgggaggccgagg | cgg |
| 25315750 | −1 | ccccacactttgggaggccg | agg |
| 25315756 | −1 | tgtaatccccacactttggg | agg |
| 25315759 | 1 | cgcctcggcctcccaaagtg | tgg |
| 25315759 | −1 | acctgtaatccccacacttt | ggg |
| 25315760 | 1 | gcctcggcctcccaaagtgt | |
| 25315760 | −1 | cacctgtaatccccacactt | tgg |
| 25315761 | 1 | cctcggcctcccaaagtgtg | ggg |
| 25315769 | 1 | tcccaaagtgtggggattac | agg |
| 25315787 | −1 | TAAATTAAggccgggtgtgg | tgg |
| 25315788 | 1 | caggtgtgagccaccacacc | cgg |
| 25315790 | −1 | AAATAAATTAAggccgggtg | tgg |
| 25315795 | −1 | TAGAAAAATAAATTAAggcc | ggg |
| 25315796 | −1 | CTAGAAAAATAAATTAAggc | cgg |
| 25315800 | −1 | CAGACTAGAAAAATAAATTA | Agg |
| 25315815 | 1 | AATTTATTTTTCTAGTCTGC | AGG |
| 25315846 | −1 | ctcataagatcataggagag | tgg |
| 25315853 | −1 | tccctacctcataagatcat | agg |
| 25315858 | 1 | cactctcctatgatcttatg | agg |
| 25315862 | 1 | ctcctatgatcttatgaggt | agg |
| 25315863 | 1 | tcctatgatcttatgaggta | ggg |
| 25315890 | −1 | ctgattgttcattataaagt | ggg |
| 25315891 | −1 | actgattgttcattataaag | tgs |
| 25315911 | 1 | aatgaacaatcagtaaagac | agg |
| 25315912 | 1 | atgaacaatcagtaaagaca | ggg |
| 25315931 | −1 | ACCCCACCTTGTATGTCATT | TGG |
| 25315936 | 1 | agataaCCAAATGACATACA | AGG |
| 25315939 | 1 | taaCCAAATGACATACAAGG | TGG |
| 25315940 | 1 | aaCCAAATGACATACAAGGT | GGG |
| 25315941 | 1 | aCCAAATGACATACAAGGTG | GGG |
| 25315954 | −1 | AAGCCTGCAGCCTCATGGGG | TGG |
| 25315955 | 1 | AAGGTGGGGTCCACCCCATG | AGG |
| 25315957 | −1 | TCCAAGCCTGCAGCCTCATG | GGG |
| 25315958 | −1 | CTCCAAGCCTGCAGCCTCAT | GGG |
| 25315959 | −1 | GCTCCAAGCCTGCAGCCTCA | TGG |
| 25315962 | 1 | GGTCCACCCCATGAGGCTGC | AGG |
| 25315967 | 1 | ACCCCATGAGGCTGCAGGCT | TGG |
| 25316015 | −1 | TGTTTCTTGTCTCAACAGGT | GGG |
| 25316016 | −1 | CTGTTTCTTGTCTCAACAGG | TGG |
| 25316019 | −1 | TTCCTGTTTCTTGTCTCAAC | AGG |
| 25316028 | 1 | CACCTGTTGAGACAAGAAAC | AGG |
| 25316033 | 1 | GTTGAGACAAGAAACAGGAA | AGG |
| 25316046 | 1 | ACAGGAAAGGCTTAAAAAAC | TGG |
| 25316070 | 1 | TTGTTATGTACAACTATCCG | TGG |
| 25316071 | 1 | TGTTATGTACAACTATCCGT | GGG |
| 25316072 | 1 | GTTATGTACAACTATCCGTG | GGG |
| 25316076 | −1 | GCCCGTTCACTGCAGCCCCA | CGG |
| 25316085 | 1 | ATCCGTGGGGCTGCAGTGAA | CGG |
| 25316086 | 1 | TCCGTGGGGCTGCAGTGAAC | GGG |
| 25316090 | 1 | TGGGGCTGCAGTGAACGGGC | TGG |
| 25316101 | 1 | TGAACGGGCTGGCAGTGCCC | AGG |
| 25316107 | 1 | GGCTGGCAGTGCCCAGGTGC | AGG |
| 25316107 | −1 | CCAGGGTTCAGCCTGCACCT | GGG |
| 25316108 | −1 | CCCAGGGTTCAGCCTGCACC | TGG |
| 25316118 | 1 | CCCAGGTGCAGGCTGAACCC | TGG |
| 25316119 | 1 | CCAGGTGCAGGCTGAACCCT | GGG |
| 25316124 | −1 | TGCTGAATGTGATTGTCCCA | GGG |
| 25316125 | −1 | ATGCTGAATGTGATTGTCCC | AGG |
| 25316142 | 1 | ACAATCACATTCAGCATCCA | AGG |
| 25316143 | 1 | CAATCACATTCAGCATCCAA | GGG |
| 25316148 | −1 | TAAGCTATTACGGGGGCCCT | TGG |
| 25316155 | −1 | CAAACATTAAGCTATTACGG | GGG |
| 25316156 | −1 | TCAAACATTAAGCTATTACG | GGG |
| 25316157 | −1 | TTCAAACATTAAGCTATTAC | GGG |
| 25316158 | −1 | ATTCAAACATTAAGCTATTA | CGG |
| 25316180 | 1 | TAATGTTTGAATTGAACCCC | TGG |
| 25316181 | 1 | AATGTTTGAATTGAACCCCT | GGG |
| 25316182 | 1 | ATGTTTGAATTGAACCCCTG | GGG |
| 25316185 | −1 | CTCCTTCAAGGCAACCCCAG | GGG |
| 25316186 | −1 | TCTCCTTCAAGGCAACCCCA | GGG |
| 25316187 | −1 | CTCTCCTTCAAGGCAACCCC | AGG |
| 25316194 | 1 | AACCCCTGGGGTTGCCTTGA | AGG |
| 25316197 | −1 | TCCACGACCTCTCTCCTTCA | AGG |
| 25316201 | 1 | GGGGTTGCCTTGAAGGAGAG | AGG |
| 25316207 | 1 | GCCTTGAAGGAGAGAGGTCG | TGG |
| 25316221 | 1 | AGGTCGTGGAAGTATGTTCA | AGG |
| 25316222 | 1 | GGTCGTGGAAGTATGTTCAA | GGG |
| 25316223 | 1 | GTCGTGGAAGTATGTTCAAG | GGG |
| 25316227 | 1 | TGGAAGTATGTTCAAGGGGT | AGG |
| 25316228 | 1 | GGAAGTATGTTCAAGGGGTA | GGG |
| 25316232 | 1 | GTATGTTCAAGGGGTAGGGA | TGG |
| 25316233 | 1 | TATGTTCAAGGGGTAGGGAT | GGG |
| 25316237 | 1 | TTCAAGGGGTAGGGATGGGC | AGG |
| 25316238 | 1 | TCAAGGGGTAGGGATGGGCA | GGG |
| 25316239 | 1 | CAAGGGGTAGGGATGGGCAG | GGG |
| 25316245 | 1 | GTAGGGATGGGCAGGGGAGA | TGG |
| 25316246 | 1 | TAGGGATGGGCAGGGGAGAT | GGG |
| 25316266 | −1 | AAGGTGGGTGGGGTAGAGCT | TGG |
| 25316276 | −1 | CTCTTGGGGCAAGGTGGGTG | GGG |
| 25316277 | −1 | TCTCTTGGGGCAAGGTGGGT | GGG |
| 25316278 | −1 | TTCTCTTGGGGCAAGGTGGG | TGG |
| 25316281 | −1 | TATTTCTCTTGGGGCAAGGT | GGG |
| 25316282 | −1 | CTATTTCTCTTGGGGCAAGG | TGG |
| 25316285 | −1 | GTTCTATTTCTCTTGGGGCA | AGG |
| 25316290 | −1 | TGAAGGTTCTATTTCTCTTG | GGG |
| 25316291 | −1 | ATGAAGGTTCTATTTCTCTT | GGG |
| 25316292 | −1 | GATGAAGGTTCTATTTCTCT | TGG |
| 25316307 | −1 | CGTTAGGCAATTAAAGATGA | AGG |
| 25316323 | −1 | ccAGCCCCAGTTTTCTCGTT | AGG |
| 25316328 | 1 | TAATTGCCTAACGAGAAAAC | TGG |
| 25316329 | 1 | AATTGCCTAACGAGAAAACT | GGG |
| 25316330 | 1 | ATTGCCTAACGAGAAAACTG | GGG |
| 25316334 | 1 | CCTAACGAGAAAACTGGGGC | Tgg |
| 25316344 | 1 | AAACTGGGGCTggccagatg | tgg |
| 25316346 | −1 | cagacatgagccaccacatc | tgg |
| 25316347 | 1 | CTGGGGCTggccagatgtgg | tgg |
| 25316373 | −1 | cctcggcctcccaaagtgct | ggg |
| 25316374 | 1 | tgtctgtaatcccagcactt | tgg |
| 25316374 | −1 | gcctcggcctcccaaagtgc | tgg |
| 25316375 | 1 | gtctgtaatcccagcacttt | ggg |
| 25316378 | 1 | tgtaatcccagcactttggg | agg |
| 25316384 | 1 | cccagcactttgggaggccg | agg |
| 25316387 | 1 | agcactttgggaggccgagg | cgg |
| 25316388 | 1 | gcactttgggaggccgaggc | ggg |
| 25316390 | −1 | ctcaagtgatctgcccgcct | cgg |
| 25316402 | 1 | cgaggcgggcagatcacttg | agg |
| 25316407 | 1 | cgggcagatcacttgaggtc | agg |
| 25316425 | 1 | tcaggagttcgagatcaccc | tgg |
| 25316431 | −1 | gggtttcaccatgttgacca | ggg |
| 25316432 | −1 | ggggtttcaccatgttgacc | agg |
| 25316434 | 1 | cgagatcaccctggtcaaca | tgg |
| 25316451 | −1 | ttgtattattaatagagacg | ggg |
| 25316452 | −1 | tttgtattattaatagagac | ggg |
| 25316453 | −1 | ttttgtattattaatagaga | cgg |
| 25316474 | 1 | taataatacaaaaattatcc | agg |
| 25316479 | 1 | atacaaaaattatccaggta | tgg |
| 25316481 | −1 | caggcatgcgccaccatacc | tgg |
| 25316482 | 1 | caaaaattatccaggtatgg | tgg |
| 25316500 | −1 | cctcaagtagctgggactac | agg |
| 25316508 | −1 | ttcttgtgcctcaagtagct | ggg |
| 25316509 | −1 | attcttgtgcctcaagtagc | tgg |
| 25316511 | 1 | cctgtagtcccagctacttg | agg |
| 25316533 | 1 | gcacaagaatcgcttgaacc | tgg |
| 25316534 | 1 | cacaagaatcgcttgaacct | ggg |
| 25316535 | 1 | acaagaatcgcttgaacctg | ggg |
| 25316536 | 1 | caagaatcgcttgaacctgg | ggg |
| 25316540 | −1 | cactgcaacctctgtccccc | agg |
| 25316543 | 1 | cgcttgaacctgggggacag | agg |
| 25316565 | −1 | ccagactggagtgcagtggt | cgg |
| 25316569 | −1 | tcgtccagactggagtgcag | tgg |
| 25316576 | 1 | ccgaccactgcactccagtc | tgg |
| 25316579 | −1 | tctcactctgtcgtccagac | tgg |
| 25316604 | −1 | ttctgtttttgtttgtgaga | tgg |
| 25316650 | 1 | aaaaGAGAGAGAgagaaaac | tgg |
| 25316653 | 1 | aGAGAGAGAgagaaaactgg | agg |
| 25316663 | 1 | agaaaactggaggctctgag | agg |
| 25316669 | 1 | ctggaggctctgagaggttg | agg |
| 25316670 | 1 | tggaggctctgagaggttga | ggg |
| 25316681 | 1 | agaggttgagggacttgccc | agg |
| 25316682 | 1 | gaggttgagggacttgccca | ggg |
| 25316687 | −1 | cttactagctgcaagaccct | ggg |
| 25316688 | −1 | acttactagctgcaagaccc | tgg |
| 25316710 | 1 | cagctagtaagtgacagagc | tgg |
| 25316711 | 1 | agctagtaagtgacagagct | ggg |
| 25316723 | 1 | acagagctgggacttgagct | tgg |
| 25316724 | 1 | cagagctgggacttgagctt | ggg |
| 25316739 | 1 | agcttgggttttctgactcc | tgg |
| 25316744 | 1 | gggttttctgactcctggtc | tgg |
| 25316746 | −1 | CAtggataatgaaccagacc | agg |
| 25316760 | 1 | ggtctggttcattatccaTG | AGG |
| 25316764 | −1 | TTTTAGTTCCCAGCACCTCA | tgg |
| 25316766 | 1 | gttcattatccaTGAGGTGC | TGG |
| 25316767 | 1 | ttcattatccaTGAGGTGCT | GGG |
| 25316791 | 1 | ACTAAAATAAGCCACAATCT | TGG |
| 25316791 | −1 | CGACGGAGATTCCAAGATTG | TGG |
| 25316808 | −1 | TGTGGGAGGGAGGGAGGCGA | CGG |
| 25316814 | −1 | CAGACATGTGGGAGGGAGGG | AGG |
| 25316817 | −1 | ACGCAGACATGTGGGAGGGA | GGG |
| 25316818 | −1 | CACGCAGACATGTGGGAGGG | AGG |
| 25316821 | −1 | AGCCACGCAGACATGTGGGA | GGG |
| 25316822 | −1 | AAGCCACGCAGACATGTGGG | AGG |
| 25316825 | −1 | AAAAAGCCACGCAGACATGT | GGG |
| 25316826 | −1 | CAAAAAGCCACGCAGACATG | TGG |
| 25316830 | 1 | CTCCCTCCCACATGTCTGCG | TGG |
| 25316838 | 1 | CACATGTCTGCGTGGCTTTT | TGG |
| 25316839 | 1 | ACATGTCTGCGTGGCTTTTT | GGG |
| 25316850 | 1 | TGGCTTTTTGGGAAAATGCC | AGG |
| 25316851 | 1 | GGCTTTTTGGGAAAATGCCA | GGG |
| 25316852 | 1 | GCTTTTTGGGAAAATGCCAG | GGG |
| 25316857 | −1 | CCCTGGCTGGTACATTCCCC | TGG |
| 25316867 | 1 | GCCAGGGGAATGTACCAGCC | AGG |
| 25316868 | 1 | CCAGGGGAATGTACCAGCCA | GGG |
| 25316870 | −1 | ACAAGGGTCCTCTCCCTGGC | TGG |
| 25316873 | 1 | GGAATGTACCAGCCAGGGAG | AGG |
| 25316874 | −1 | GAAAACAAGGGTCCTCTCCC | TGG |
| 25316886 | −1 | AAGGGCCATGAGGAAAACAA | GGG |
| 25316887 | −1 | GAAGGGCCATGAGGAAAACA | AGG |
| 25316892 | 1 | GAGGACCCTTGTTTTCCTCA | TGG |
| 25316896 | −1 | CATTGCCAGGAAGGGCCATG | AGG |
| 25316902 | 1 | GTTTTCCTCATGGCCCTTCC | TGG |
| 25316904 | −1 | AGTAGTGCCATTGCCAGGAA | GGG |
| 25316905 | −1 | CAGTAGTGCCATTGCCAGGA | AGG |
| 25316908 | 1 | CTCATGGCCCTTCCTGGCAA | TGG |
| 25316909 | −1 | GTGTCAGTAGTGCCATTGCC | AGG |
| 25316931 | −1 | TCAGGGACAAAAAGGACTGT | CGG |
| 25316939 | −1 | AGAGGTCATCAGGGACAAAA | AGG |
| 25316948 | −1 | TCAGGCAGCAGAGGTCATCA | GGG |
| 25316949 | −1 | ATCAGGCAGCAGAGGTCATC | AGG |
| 25316957 | −1 | ACTTGGGCATCAGGCAGCAG | AGG |
| 25316966 | −1 | GAGGTGGTCACTTGGGCATC | AGG |
| 25316973 | −1 | CAAAGCAGAGGTGGTCACTT | GGG |
| 25316974 | −1 | ACAAAGCAGAGGTGGTCACT | TGG |
| 25316982 | −1 | TAGAAATGACAAAGCAGAGG | TGG |
| 25316985 | −1 | TCCTAGAAATGACAAAGCAG | AGG |
| 25316995 | 1 | ACCTCTGCTTTGTCATTTCT | AGG |
| 25317000 | 1 | TGCTTTGTCATTTCTAGGAT | TGG |
| 25317008 | 1 | CATTTCTAGGATTGGCTTCC | AGG |
| 25317015 | −1 | CCCCAATGCTGAGGAGGACC | TGG |
| 25317021 | −1 | TGAGTTCCCCAATGCTGAGG | AGG |
| 25317024 | 1 | TTCCAGGTCCTCCTCAGCAT | TGG |
| 25317024 | −1 | AGCTGAGTTCCCCAATGCTG | AGG |
| 25317025 | 1 | TCCAGGTCCTCCTCAGCATT | GGG |
| 25317026 | 1 | CCAGGTCCTCCTCAGCATTG | GGG |
| 25317038 | 1 | CAGCATTGGGGAACTCAGCT | TGG |
| 25317050 | −1 | AGACATGAGAGCTATCACGA | TGG |
| 25317063 | 1 | ATCGTGATAGCTCTCATGTC | TGG |
| 25317075 | 1 | CTCATGTCTGGTCTCCTGAC | AGG |
| 25317078 | −1 | TGGCCTCACACTGACCTGTC | AGG |
| 25317086 | 1 | TCTCCTGACAGGTCAGTGTG | AGG |
| 25317098 | −1 | TGGCAATGGTGGAAGAAAGG | TGG |
| 25317101 | −1 | TCCTGGCAATGGTGGAAGAA | AGG |
| 25317109 | −1 | GTGCTGTGTCCTGGCAATGG | TGG |
| 25317111 | 1 | ACCTTTCTTCCACCATTGCC | AGG |
| 25317112 | −1 | TGGGTGCTGTGTCCTGGCAA | TGG |
| 25317118 | −1 | TGGACGTGGGTGCTGTGTCC | TGG |
| 25317131 | −1 | GCAGGGTGCGCTCTGGACGT | GGG |
| 25317132 | −1 | GGCAGGGTGCGCTCTGGACG | TGG |
| 25317138 | −1 | CCACACGGCAGGGTGCGCTC | TGG |
| 25317148 | −1 | AGACATCCAGCCACACGGCA | GGG |
| 25317149 | 1 | CCAGAGCGCACCCTGCCGTG | TGG |
| 25317149 | −1 | TAGACATCCAGCCACACGGC | AGG |
| 25317153 | 1 | AGCGCACCCTGCCGTGTGGC | TGG |
| 25317153 | −1 | CACATAGACATCCAGCCACA | CGG |
| 25317176 | −1 | gatcctCAGGGAAGGAGATG | GGG |
| 25317177 | −1 | tgatcctCAGGGAAGGAGAT | GGG |
| 25317178 | −1 | gtgatcctCAGGGAAGGAGA | TGG |
| 25317184 | 1 | GTGCCCCATCTCCTTCCCTG | agg |
| 25317184 | −1 | aattatgtgatcctCAGGGA | AGG |
| 25317188 | −1 | ctgaaattatgtgatcctCA | GGG |
| 25317189 | −1 | tctgaaattatgtgatcctC | AGG |
| 25317205 | 1 | ggatcacataatttcagaat | tgg |
| 25317210 | 1 | acataatttcagaattggaa | agg |
| 25317220 | 1 | agaattggaaaggttcttag | agg |
| 25317235 | −1 | tcacagtccacattagcagc | agg |
| 25317239 | 1 | gaggtcacctgctgctaatg | tgg |
| 25317248 | 1 | tgctgctaatgtggactgtg | agg |
| 25317253 | 1 | ctaatgtggactgtgaggcc | agg |
| 25317254 | 1 | taatgtggactgtgaggcca | ggg |
| 25317258 | 1 | gtggactgtgaggccagggc | agg |
| 25317259 | 1 | tggactgtgaggccagggca | ggg |
| 25317260 | −1 | gggatgtcccttccctgccc | tgg |
| 25317263 | 1 | ctgtgaggccagggcaggga | agg |
| 25317264 | 1 | tgtgaggccagggcagggaa | ggg |
| 25317276 | 1 | gcagggaagggacatccctg | agg |
| 25317280 | −1 | tcaccctacttataacctca | ggg |
| 25317281 | −1 | ctcaccctacttataacctc | agg |
| 25317287 | 1 | acatccctgaggttataagt | agg |
| 25317288 | 1 | catccctgaggttataagta | ggg |
| 25317295 | 1 | gaggttataagtagggtgag | tgg |
| 25317321 | 1 | cgttgcagacttttgaaccc | agg |
| 25317322 | 1 | gttgcagacttttgaaccca | ggg |
| 25317326 | 1 | cagacttttgaacccagggc | tgg |
| 25317327 | −1 | tgagtgtgatcaccagccct | ggg |
| 25317328 | −1 | ctgagtgtgatcaccagccc | tgg |
| 25317364 | −1 | TTGGGTGTAAGGATTTTCTC | GGG |
| 25317365 | −1 | TTTGGGTGTAAGGATTTTCT | CGG |
| 25317375 | −1 | AAGGTAGGCTTTTGGGTGTA | AGG |
| 25317413 | −1 | gttgaataaaATAGTATTAT | GGG |
| 25317414 | −1 | tgttgaataaaATAGTATTA | TGG |
| 25317453 | −1 | ccccagtgcctggctcatag | tgg |
| 25317456 | 1 | ttcaatatccactatgagcc | agg |
| 25317462 | 1 | atccactatgagccaggcac | tgg |
| 25317463 | 1 | tccactatgagccaggcact | ggg |
| 25317463 | −1 | actgctgtgtccccagtgcc | tgg |
| 25317464 | 1 | ccactatgagccaggcactg | ggg |
| 25317500 | −1 | aggtcaattccatggggtca | ggg |
| 25317501 | −1 | aaggtcaattccatggggtc | agg |
| 25317502 | 1 | aaacaaattccctgacccca | tgg |
| 25317506 | −1 | actagaaggtcaattccatg | ggg |
| 25317507 | −1 | cactagaaggtcaattccat | ggg |
| 25317508 | −1 | ccactagaaggtcaattcca | tgg |
| 25317519 | 1 | ccatggaattgaccttctag | tgg |
| 25317520 | 1 | catggaattgaccttctagt | ggg |
| 25317520 | −1 | taataccttcccccactaga | agg |
| 25317521 | 1 | atggaattgaccttctagtg | ggg |
| 25317522 | 1 | tggaattgaccttctagtgg | ggg |
| 25317526 | 1 | attgaccttctagtggggga | agg |
| 25317570 | 1 | taagtgtctactacgccaga | tgg |
| 25317571 | 1 | aagtgtctactacgccagat | ggg |
| 25317574 | −1 | cacagccacttcttcccatc | tgg |
| 25317580 | 1 | ctacgccagatgggaagaag | tgg |
| 25317623 | 1 | agagaaacatagagtcaatg | tgg |
| 25317624 | 1 | gagaaacatagagtcaatgt | ggg |
| 25317628 | 1 | aacatagagtcaatgtggga | tgg |
| 25317629 | 1 | acatagagtcaatgtgggat | ggg |
| 25317630 | 1 | catagagtcaatgtgggatg | ggg |
| 25317642 | 1 | gtgggatggggtgttctttt | agg |
| 25317643 | 1 | tgggatggggtgttctttta | ggg |
| 25317644 | 1 | gggatggggtgttcttttag | ggg |
| 25317645 | 1 | ggatggggtgttcttttagg | ggg |
| 25317646 | 1 | gatggggtgttcttttaggg | ggg |
| 25317649 | 1 | ggggtgttcttttagggggg | tgg |
| 25317654 | 1 | gttcttttaggggggtggtc | agg |
| 25317655 | 1 | ttcttttaggggggtggtca | ggg |
| 25317702 | −1 | tatctccctcctcttcattg | ggg |
| 25317703 | −1 | atatctccctcctcttcatt | ggg |
| 25317704 | 1 | aagcagagaccccaatgaag | agg |
| 25317704 | −1 | catatctccctcctcttcat | tgg |
| 25317707 | 1 | cagagaccccaatgaagagg | agg |
| 25317708 | 1 | agagaccccaatgaagagga | ggg |
| 25317732 | 1 | gatatgcgatgcatttagtt | agg |
| 25317733 | 1 | atatgcgatgcatttagtta | ggg |
| 25317734 | 1 | tatgcgatgcatttagttag | ggg |
| 25317755 | −1 | cacttgctatcctattttca | tgg |
| 25317756 | 1 | gaagaacattccatgaaaat | agg |
| 25317772 | 1 | aaataggatagcaagtgcaa | agg |
| 25317784 | −1 | caaagcatgctgctgtctca | ggg |
| 25317785 | −1 | acaaagcatgctgctgtctc | agg |
| 25317805 | 1 | gcagcatgctttgtgtgttg | agg |
| 25317806 | 1 | cagcatgctttgtgtgttga | ggg |
| 25317816 | 1 | tgtgtgttgagggaacagta | agg |
| 25317828 | 1 | gaacagtaaggagaccagtg | tgg |
| 25317831 | −1 | tccattcacaccaaccacac | tgg |
| 25317832 | 1 | agtaaggagaccagtgtggt | tgg |
| 25317841 | 1 | accagtgtggttggtgtgaa | tgg |
| 25317851 | 1 | ttggtgtgaatggagtgaga | agg |
| 25317860 | 1 | atggagtgagaaggagcagc | agg |
| 25317861 | 1 | tggagtgagaaggagcagca | ggg |
| 25317862 | 1 | ggagtgagaaggagcagcag | ggg |
| 25317868 | 1 | agaaggagcagcaggggttg | agg |
| 25317869 | 1 | gaaggagcagcaggggttga | ggg |
| 25317877 | 1 | agcaggggttgagggcagaa | tgg |
| 25317885 | 1 | ttgagggcagaatggtagtg | agg |
| 25317891 | 1 | gcagaatggtagtgaggagc | agg |
| 25317903 | −1 | tggcttcccatcttttataa | ggg |
| 25317904 | −1 | gtggcttcccatcttttata | agg |
| 25317907 | 1 | gagcaggcccttataaaaga | tgg |
| 25317908 | 1 | agcaggcccttataaaagat | ggg |
| 25317918 | 1 | tataaaagatgggaagccac | tgg |
| 25317923 | −1 | CTTTGTTGaaagatctccag | tgg |
| 25317935 | 1 | cactggagatctttCAACAA | AGG |
| 25317936 | 1 | actggagatctttCAACAAA | GGG |
| 25317937 | 1 | ctggagatctttCAACAAAG | GGG |
| 25317987 | 1 | AATAGAACAGCAAAAAATCT | AGG |
| 25317988 | 1 | ATAGAACAGCAAAAAATCTA | GGG |
| 25317989 | 1 | TAGAACAGCAAAAAATCTAG | GGG |
| 25318014 | −1 | ACCTGGCATATAAGTAAAAC | TGG |
| 25318024 | 1 | GCCAGTTTTACTTATATGCC | AGG |
| 25318031 | −1 | cctagccACATATTTTCACC | TGG |
| 25318037 | 1 | ATATGCCAGGTGAAAATATG | Tgg |
| 25318042 | 1 | CCAGGTGAAAATATGTggct | agg |
| 25318050 | 1 | AAATATGTggctaggtgcag | tgg |
| 25318068 | −1 | tcccaaactgctgcaattac | agg |
| 25318077 | 1 | tacctgtaattgcagcagtt | tgg |
| 25318078 | 1 | acctgtaattgcagcagttt | ggg |
| 25318090 | 1 | agcagtttgggagaccgaag | tgg |
| 25318091 | 1 | gcagtttgggagaccgaagt | ggg |
| 25318093 | −1 | ctcagatgatctgcccactt | cgg |
| 25318110 | 1 | tgggcagatcatctgagatc | agg |
| 25318127 | 1 | atcaggattcaagaccagca | tgg |
| 25318130 | −1 | tttcaccatgttggccatgc | tgg |
| 25318136 | 1 | caagaccagcatggccaaca | tgg |
| 25318139 | −1 | gagatggggtttcaccatgt | tgg |
| 25318153 | −1 | tttaatttttagtagagatg | ggg |
| 25318154 | −1 | ttttaatttttagtagagat | ggg |
| 25318155 | −1 | tttttaatttttagtagaga | tgg |
| 25318176 | 1 | taaaaattaaaaaataagcc | agg |
| 25318181 | 1 | attaaaaaataagccaggcg | tgg |
| 25318183 | −1 | ctgggatccaacaccacgcc | tgg |
| 25318187 | 1 | aaataagccaggcgtggtgt | tgg |
| 25318201 | −1 | cctcagcctcccaagtagct | ggg |
| 25318202 | 1 | ggtgttggatcccagctact | tgg |
| 25318202 | −1 | gcctcagcctoccaagtagc | tgg |
| 25318203 | 1 | gtgttggatcccagctactt | ggg |
| 25318206 | 1 | ttggatcccagctacttggg | agg |
| 25318212 | 1 | cccagctacttgggaggctg | agg |
| 25318234 | 1 | gcagtagaattgcttgaacc | cgg |
| 25318235 | 1 | cagtagaattgcttgaaccc | ggg |
| 25318238 | 1 | tagaattgcttgaacccggg | agg |
| 25318241 | −1 | cactgcaacctctgcctccc | ggg |
| 25318242 | −1 | tcactgcaacctctgcctcc | cgg |
| 25318244 | 1 | tgcttgaacccgggaggcag | agg |
| 25318266 | −1 | tttttttttAGACAGAGtct | cgg |
| 25318302 | 1 | aaaaaagaaaaTACACATTC | Agg |
| 25318307 | 1 | agaaaaTACACATTCAggcc | agg |
| 25318314 | −1 | caggcgtgagccactgcacc | tgg |
| 25318315 | 1 | CACATTCAggccaggtgcag | tgg |
| 25318333 | −1 | tcccaaagtgctgggattac | agg |
| 25318341 | −1 | tctcagcctcccaaagtgct | ggg |
| 25318342 | 1 | cgcctgtaatcccagcactt | tgg |
| 25318342 | −1 | gtctcagcctcccaaagtgc | tgg |
| 25318343 | 1 | gcctgtaatcccagcacttt | ggg |
| 25318346 | 1 | tgtaatcccagcactttggg | agg |
| 25318356 | 1 | gcactttgggaggctgagac | agg |
| 25318370 | 1 | tgagacaggtagatcacttg | agg |
| 25318375 | 1 | caggtagatcacttgaggtc | agg |
| 25318396 | −1 | ttttgccatgttggtcaggc | tgg |
| 25318400 | −1 | agggttttgccatgttggtc | agg |
| 25318402 | 1 | cgagaccagcctgaccaaca | tgg |
| 25318405 | −1 | gagacagggttttgccatgt | tgg |
| 25318419 | −1 | ttgtatttctggtagagaca | ggg |
| 25318420 | −1 | tttgtatttctggtagagac | agg |
| 25318430 | −1 | ctggctaatttttgtatttc | tgg |
| 25318442 | 1 | cagaaatacaaaaattagcc | agg |
| 25318447 | 1 | atacaaaaattagccaggcg | tgg |
| 25318449 | −1 | caggcacacgccaccacgcc | tgg |
| 25318450 | 1 | caaaaattagccaggcgtgg | tgg |
| 25318468 | −1 | tccccagtagctgggactac | agg |
| 25318476 | 1 | gtgcctgtagtcccagctac | tgg |
| 25318476 | −1 | cttcagcctccccagtagct | ggg |
| 25318477 | 1 | tgcctgtagtcccagctact | ggg |
| 25318477 | −1 | acttcagcctccccagtagc | tgg |
| 25318478 | 1 | gcctgtagtcccagctactg | ggg |
| 25318481 | 1 | tgtagtcccagctactgggg | agg |
| 25318491 | 1 | gctactggggaggctgaagt | agg |
| 25318492 | 1 | ctactggggaggctgaagta | ggg |
| 25318493 | 1 | tactggggaggctgaagtag | ggg |
| 25318498 | 1 | gggaggctgaagtaggggaa | tgg |
| 25318510 | 1 | taggggaatggcttgacccc | agg |
| 25318513 | 1 | gggaatggcttgaccccagg | agg |
| 25318515 | −1 | actataacctccacctcctg | ggg |
| 25318516 | 1 | aatggcttgaccccaggagg | tgg |
| 25318516 | −1 | cactataacctccacctcct | ggg |
| 25318517 | −1 | tcactataacctccacctcc | tgg |
| 25318519 | 1 | ggcttgaccccaggaggtgg | agg |
| 25318535 | 1 | gtggaggttatagtgagtcg | agg |
| 25318552 | −1 | tcacctaggctggagggcag | tgg |
| 25318558 | −1 | actctgtcacctaggctgga | ggg |
| 25318559 | −1 | cactctgtcacctaggctgg | agg |
| 25318560 | 1 | gcaccactgccctccagcct | agg |
| 25318562 | −1 | tctcactctgtcacctaggc | tgg |
| 25318566 | −1 | acagtctcactctgtcacct | agg |
| 25318619 | −1 | TAAAGGTGAACAGTTCTGGA | TGG |
| 25318623 | −1 | AGAATAAAGGTGAACAGTTC | TGG |
| 25318636 | −1 | GATGTTTGCTTGTAGAATAA | AGG |
| 25318656 | 1 | TACAAGCAAACATCTTTTAT | TGG |
| 25318675 | −1 | CTGCTTAGGGACACATATAT | GGG |
| 25318676 | −1 | CCTGCTTAGGGACACATATA | TGG |
| 25318687 | 1 | CCATATATGTGTCCCTAAGC | AGG |
| 25318688 | −1 | TGGCATTCACCTCCTGCTTA | GGG |
| 25318689 | −1 | TTGGCATTCACCTCCTGCTT | AGG |
| 25318690 | 1 | TATATGTGTCCCTAAGCAGG | AGG |
| 25318708 | −1 | TACGCCATTTGTCTCTTATT | TGG |
| 25318715 | 1 | AATGCCAAATAAGAGACAAA | TGG |
| 25318745 | 1 | cactatgagttgtgtgacgt | tgg |
| 25318746 | 1 | actatgagttgtgtgacgtt | ggs |
| 25318772 | −1 | gaagctaaccaaggctcaga | 555 |
| 25318773 | −1 | agaagctaaccaaggctcag | agg |
| 25318775 | 1 | actttactccctctgagcct | tgg |
| 25318781 | −1 | ttttacagagaagctaacca | agg |
| 25318799 | 1 | tagcttctctgtaaaatgaa | agg |
| 25318806 | 1 | tctgtaaaatgaaaggatta | tgg |
| 25318818 | 1 | aaggattatggtaactaagc | tgg |
| 25318833 | −1 | TACAGTTtgttaaagctgga | agg |
| 25318837 | −1 | TCCATACAGTTtgttaaagc | tgg |
| 25318847 | 1 | tccagctttaacaAACTGTA | TGG |
| 25318850 | 1 | agctttaacaAACTGTATGG | AGG |
| 25318860 | 1 | AACTGTATGGAGGTACTTTT | TGG |
| 25318870 | 1 | AGGTACTTTTTGGAGTTACC | TGG |
| 25318871 | 1 | GGTACTTTTTGGAGTTACCT | GGG |
| 25318877 | −1 | CTCACACTCAAAAATTACCC | AGG |
| 25318893 | 1 | GTAATTTTTGAGTGTGAGAT | TGG |
| 25318922 | 1 | TTGCTTTAATATACCATGTC | TGG |
| 25318924 | −1 | CAAAAAGCTAAGGCCAGACA | TGG |
| 25318934 | −1 | AAAGACTCTGCAAAAAGCTA | AGG |
| 25318960 | 1 | GAGTCTTTGTGAAGAAGCAG | AGG |
| 25318963 | 1 | TCTTTGTGAAGAAGCAGAGG | CGG |
| 25318988 | −1 | ACGAACTGAACGTTAACTTA | CGG |
| 25319001 | 1 | GTAAGTTAACGTTCAGTTCG | TGG |
| 25319008 | 1 | AACGTTCAGTTCGTGGCAGC | TGG |
| 25319022 | 1 | GGCAGCTGGCAATCCAACCC | TGG |
| 25319023 | 1 | GCAGCTGGCAATCCAACCCT | GGG |
| 25319024 | −1 | CCGGCAGCCTTTCCCAGGGT | TGG |
| 25319028 | 1 | TGGCAATCCAACCCTGGGAA | AGG |
| 25319028 | −1 | AAATCCGGCAGCCTTTCCCA | GGG |
| 25319029 | −1 | TAAATCCGGCAGCCTTTCCC | AGG |
| 25319035 | 1 | CCAACCCTGGGAAAGGCTGC | CGG |
| 25319043 | −1 | CCTTGCATTTTTGCTAAATC | CGG |
| 25319054 | 1 | CCGGATTTAGCAAAAATGCA | AGG |
| 25319083 | 1 | TTTTTaaatttgaaatgaat | tgg |
| 25319084 | 1 | TTTTaaatttgaaatgaatt | ggg |
| 25319099 | −1 | agggttgccaaataaaatgc | agg |
| 25319103 | 1 | tgggtatcctgcattttatt | tgg |
| 25319117 | 1 | tttatttggcaaccctGTCC | TGG |
| 25319118 | 1 | ttatttggcaaccctGTCCT | GGG |
| 25319118 | −1 | ATAGTGTGAGTCCCAGGACa | ggg |
| 25319119 | −1 | AATAGTGTGAGTCCCAGGAC | agg |
| 25319124 | −1 | CAGTGAATAGTGTGAGTCCC | AGG |
| 25319145 | 1 | ACACTATTCACTGTTATCAC | TGG |
| 25319159 | 1 | TATCACTGGTATGTTCAAAG | TGG |
| 25319181 | −1 | CTGGTACTTTGCAAGACAGA | GGG |
| 25319182 | −1 | CCTGGTACTTTGCAAGACAG | AGG |
| 25319193 | 1 | CCTCTGTCTTGCAAAGTACC | AGG |
| 25319196 | 1 | CTGTCTTGCAAAGTACCAGG | AGG |
| 25319200 | −1 | AAGAATAAGAAAAGACCTCC | TGG |
| 25319217 | 1 | GGTCTTTTCTTATTCTTCAC | TGG |
| 25319238 | 1 | GGAGTCAAAAAAGAGAATAG | AGG |
| 25319269 | −1 | TTGTTGGTCTTAACTCTTAA | AGG |
| 25319285 | −1 | ATGTAAAGAAGAAAACTTGT | TGG |
| 25319321 | 1 | TGTTTTTGACATGAGCAAAC | TGG |
| 25319339 | 1 | ACTGGTGATTAAAAACAACT | TGg |
| 25319340 | 1 | CTGGTGATTAAAAACAACTT | Ggg |
| 25319343 | 1 | GTGATTAAAAACAACTTGgg | tgg |
| 25319369 | −1 | cctcagcttcccaaggtgct | ggg |
| 25319370 | 1 | tacttgtaatcccagcacct | tgg |
| 25319370 | −1 | acctcagcttcccaaggtgc | tgg |
| 25319371 | 1 | acttgtaatcccagcacctt | ggg |
| 25319376 | −1 | tctcccacctcagcttccca | agg |
| 25319380 | 1 | cccagcaccttgggaagctg | agg |
| 25319383 | 1 | agcaccttgggaagctgagg | tgg |
| 25319384 | 1 | gcaccttgggaagctgaggt | ggg |
| 25319398 | 1 | tgaggtgggagaatagcttg | agg |
| 25319403 | 1 | tgggagaatagcttgaggcc | agg |
| 25319410 | −1 | gttgccctggcttgaactcc | tgg |
| 25319416 | 1 | tgaggccaggagttcaagcc | agg |
| 25319417 | 1 | gaggccaggagttcaagcca | ggg |
| 25319423 | −1 | ggggtctcactatgttgccc | tgg |
| 25319442 | −1 | ttgtatcttttgtagagatg | ggg |
| 25319443 | −1 | tttgtatcttttgtagagat | ggg |
| 25319444 | −1 | ttttgtatcttttgtagaga | tgg |
| 25319465 | 1 | aaaagatacaaaaattagcc | agg |
| 25319470 | 1 | atacaaaaattagccaggcg | tgg |
| 25319472 | −1 | tacaggtgtaccaccacgcc | tgg |
| 25319473 | 1 | caaaaattagccaggcgtgg | tgg |
| 25319489 | −1 | tccagagcagctgggactac | agg |
| 25319497 | −1 | tctcagcctccagagcagct | ggg |
| 25319498 | −1 | atctcagcctccagagcagc | tgg |
| 25319499 | 1 | acctgtagtcccagctgctc | tgg |
| 25319502 | 1 | tgtagtcccagctgctctgg | agg |
| 25319511 | 1 | agctgctctggaggctgaga | tgg |
| 25319512 | 1 | gctgctctggaggctgagat | ggg |
| 25319515 | 1 | gctctggaggctgagatggg | agg |
| 25319530 | 1 | atgggaggatcagttgagct | tgg |
| 25319531 | 1 | tgggaggatcagttgagctt | ggg |
| 25319534 | 1 | gaggatcagttgagcttggg | agg |
| 25319573 | −1 | ttgtccaggctggagtgcag | tgg |
| 25319580 | 1 | catgccactgcactccagcc | tgg |
| 25319583 | −1 | tcttgctctgttgtccaggc | tgg |
| 25319587 | −1 | agggtcttgctctgttgtcc | agg |
| 25319606 | −1 | ttgtttccttttttgagaca | ggg |
| 25319607 | −1 | tttgtttccttttttgagac | agg |
| 25319611 | 1 | gcaagaccctgtctcaaaaa | agg |
| 25319627 | 1 | aaaaaggaaacaaaacaaCT | TGG |
| 25319634 | 1 | aaacaaaacaaCTTGGACAA | TGG |
| 25319638 | 1 | aaaacaaCTTGGACAATGGA | AGG |
| 25319639 | 1 | aaacaaCTTGGACAATGGAA | GGG |
| 25319640 | 1 | aacaaCTTGGACAATGGAAG | GGG |
| 25319641 | 1 | acaaCTTGGACAATGGAAGG | GGG |
| 25319661 | −1 | GGTGCAATTTTGGCTGCTTG | AGG |
| 25319671 | −1 | GAGTCCATTTGGTGCAATTT | TGG |
| 25319678 | 1 | GCAGCCAAAATTGCACCAAA | TGG |
| 25319682 | −1 | TTGTCTTCTGGGAGTCCATT | TGG |
| 25319693 | −1 | AAATTAAATGCTTGTCTTCT | GGG |
| 25319694 | −1 | CAAATTAAATGCTTGTCTTC | TGG |
| 25319724 | 1 | TTTGTTAATTGAGCCCTCTA | Tgg |
| 25319725 | 1 | TTGTTAATTGAGCCCTCTAT | ggg |
| 25319726 | −1 | aatacagacaggcccATAGA | GGG |
| 25319727 | −1 | aaatacagacaggcccATAG | AGG |
| 25319737 | −1 | tttcttaaataaatacagac | agg |
| 25319764 | −1 | acccaataactatgcttgat | agg |
| 25319773 | 1 | atcctatcaagcatagttat | tgg |
| 25319774 | 1 | tcctatcaagcatagttatt | ggg |
| 25319788 | 1 | gttattgggtttctcagccc | agg |
| 25319794 | −1 | ctgctatttctaatctacct | ggg |
| 25319795 | −1 | tctgctatttctaatctacc | tgg |
| 25319813 | 1 | gattagaaatagcagattag | agg |
| 25319816 | 1 | tagaaatagcagattagagg | tgg |
| 25319817 | 1 | agaaatagcagattagaggt | ggg |
| 25319822 | 1 | tagcagattagaggtgggct | agg |
| 25319832 | 1 | gaggtgggctaggtttctag | agg |
| 25319853 | −1 | ctttcacttctaacttctgc | tgg |
| 25319882 | 1 | gaaagcaaagagcctaacag | agg |
| 25319883 | −1 | agaatttctcttcctctgtt | agg |
| 25319939 | 1 | cagttttgctcttgttgccc | agg |
| 25319943 | 1 | tttgctcttgttgcccaggc | tgg |
| 25319945 | −1 | gcgccattgcactccagcct | ggg |
| 25319946 | −1 | agcgccattgcactccagcc | tgg |
| 25319953 | 1 | ttgcccaggctggagtgcaa | tgg |
| 25319964 | 1 | ggagtgcaatggcgctatct | cgg |
| 25319986 | −1 | cacttgaacccaggaggctg | agg |
| 25319988 | 1 | tcactacaacctcagcctcc | tgg |
| 25319989 | 1 | cactacaacctcagcctcct | ggg |
| 25319992 | −1 | gagaatcacttgaacccagg | agg |
| 25319995 | −1 | caggagaatcacttgaaccc | agg |
| 25320014 | −1 | gctactcgggaggctgaggc | agg |
| 25320018 | −1 | cccagctactcgggaggctg | agg |
| 25320024 | −1 | tgtaatcccagctactcggg | agg |
| 25320027 | −1 | gcctgtaatcccagctactc | ggg |
| 25320028 | 1 | gcctcagcctcccgagtagc | tgg |
| 25320028 | −1 | tgcctgtaatcccagctact | cgg |
| 25320029 | 1 | cctcagcctcccgagtagct | ggg |
| 25320037 | 1 | tcccgagtagctgggattac | agg |
| 25320055 | −1 | acaaaattagccgggtgtgg | tgg |
| 25320056 | 1 | caggcatgcaccaccacacc | cgg |
| 25320058 | −1 | aatacaaaattagccgggtg | tgg |
| 25320063 | −1 | ctaaaaatacaaaattagcc | ggg |
| 25320064 | −1 | actaaaaatacaaaattagc | cgg |
| 25320084 | 1 | tttgtatttttagtagagac | agg |
| 25320085 | 1 | ttgtatttttagtagagaca | ggg |
| 25320099 | 1 | gagacagggtttctccatgt | tgg |
| 25320102 | −1 | cgagaccagcatgaccaaca | tgg |
| 25320108 | 1 | tttctccatgttggtcatgc | tgg |
| 25320129 | 1 | ggtctcgaactcctgacctc | agg |
| 25320129 | −1 | tgggcggatcacctgaggtc | agg |
| 25320134 | −1 | caaggtgggcggatcacctg | agg |
| 25320145 | −1 | ctttgggaggccaaggtggg | cgg |
| 25320146 | 1 | ctcaggtgatccgcccacct | tgg |
| 25320148 | −1 | gcactttgggaggccaaggt | ggg |
| 25320149 | −1 | agcactttgggaggccaagg | tgg |
| 25320152 | −1 | cccagcactttgggaggcca | agg |
| 25320158 | −1 | tgtaatcccagcactttggg | agg |
| 25320161 | −1 | ccctgtaatcccagcacttt | ggg |
| 25320162 | 1 | accttggcctcccaaagtgc | tgg |
| 25320162 | −1 | tccctgtaatcccagcactt | tgg |
| 25320163 | 1 | ccttggcctcccaaagtgct | ggg |
| 25320171 | 1 | tcccaaagtgctgggattac | agg |
| 25320172 | 1 | cccaaagtgctgggattaca | ggg |
| 25320189 | −1 | aatttgtcggccggtcgcag | tgg |
| 25320190 | 1 | cagggataagccactgcgac | cgg |
| 25320198 | −1 | agttttaagaatttgtcggc | cgg |
| 25320202 | −1 | gtccagttttaagaatttgt | cgg |
| 25320211 | 1 | ggccgacaaattcttaaaac | tgg |
| 25320234 | 1 | acacaagaacacaaaacgcT | TGG |
| 25320235 | 1 | cacaagaacacaaaacgcTT | GGG |
| 25320270 | −1 | AAAAGGTGTGTAGCTGTGGA | GGG |
| 25320271 | −1 | GAAAAGGTGTGTAGCTGTGG | AGG |
| 25320274 | −1 | GTGGAAAAGGTGTGTAGCTG | TGG |
| 25320287 | −1 | CGTGCCATATAACGTGGAAA | AGG |
| 25320293 | −1 | TTATAACGTGCCATATAACG | TGG |
| 25320294 | 1 | CACACCTTTTCCACGTTATA | TGG |
| 25320308 | 1 | GTTATATGGCACGTTATAAG | TGG |
| 25320309 | 1 | TTATATGGCACGTTATAAGT | GGG |
| 25320324 | 1 | TAAGTGGGTGTTCCTAGTGA | TGG |
| 25320325 | −1 | aaaaaaTCAGAACCATCACT | AGG |
| 25320412 | −1 | CTGAGGCTTACTCATCACTG | AGG |
| 25320429 | −1 | ATGAATTTTCCAGATAGCTG | AGG |
| 25320431 | 1 | ATGAGTAAGCCTCAGCTATC | TGG |
| 25320445 | 1 | GCTATCTGGAAAATTCATGC | AGG |
| 25320459 | −1 | AATTACTCAGTAACGATCTC | TGG |
| 25320492 | 1 | TCAAGCTAACTGCGTCATGC | TGG |
| 25320509 | −1 | TTAGCTGATATTGGCATGCA | GGG |
| 25320510 | −1 | TTTAGCTGATATTGGCATGC | AGG |
| 25320518 | −1 | GTGCTGCTTTTAGCTGATAT | TGG |
| 25320538 | 1 | GCTAAAAGCAGCACCACGAA | AGG |
| 25320539 | 1 | CTAAAAGCAGCACCACGAAA | GGG |
| 25320540 | −1 | AGATTCGTATTTCCCTTTCG | TGG |
| 25320576 | −1 | CCAGTGTCGTTAACAAGAAT | GGG |
| 25320577 | −1 | TCCAGTGTCGTTAACAAGAA | TGG |
| 25320587 | 1 | CCCATTCTTGTTAACGACAC | TGG |
| 25320609 | −1 | GATTTATCTGTGTATTATTA | AGG |
| 25320627 | 1 | AATACACAGATAAATCTATC | AGG |
| 25320646 | 1 | GCTTCCTTTCACAGGAAGCA | AGG |
| 25320653 | 1 | ATTTCCTTGCTTCCTGTGAA | AGG |
| 25320654 | −1 | GAATGAGTGCTTCCTTTCAC | AGG |
| 25320676 | −1 | GATGAATTTCACAGGACACA | TGG |
| 25320684 | −1 | TGAAGTTGGATGAATTTCAC | AGG |
| 25320697 | 1 | TGTGAAATTCATCCAACTTC | AGG |
| 25320698 | −1 | TTCCTCCAGCTTCCTGAAGT | TGG |
| 25320704 | 1 | TTCATCCAACTTCAGGAAGC | TGG |
| 25320707 | 1 | ATCCAACTTCAGGAAGCTGG | AGG |
| 25320718 | 1 | GGAAGCTGGAGGAATACATA | TGG |
| 25320730 | −1 | TACTCTCTGCCCAGATAGCT | TGG |
| 25320731 | 1 | ATACATATGGCCAAGCTATC | TGG |
| 25320732 | 1 | TACATATGGCCAAGCTATCT | GGG |
| 25320747 | 1 | TATCTGGGCAGAGAGTAGAC | AGG |
| 25320748 | 1 | ATCTGGGCAGAGAGTAGACA | GGG |
| 25320753 | 1 | GGCAGAGAGTAGACAGGGAA | TGG |
| 25320756 | 1 | AGAGAGTAGACAGGGAATGG | Agg |
| 25320760 | 1 | AGTAGACAGGGAATGGAggt | tgg |
| 25320761 | 1 | GTAGACAGGGAATGGAggtt | ggg |
| 25320769 | 1 | GGAATGGAggttgggcacag | tgg |
| 25320787 | −1 | ttctaaatggctgcgattac | agg |
| 25320800 | 1 | tgtaatcgcagccatttaga | agg |
| 25320800 | −1 | gcccgcctttgccttctaaa | tgg |
| 25320806 | 1 | cgcagccatttagaaggcaa | agg |
| 25320809 | 1 | agccatttagaaggcaaagg | cgg |
| 25320810 | 1 | gccatttagaaggcaaaggc | ggg |
| 25320829 | 1 | cgggcagatcacttgagctc | agg |
| 25320847 | 1 | tcaggtgttcaagaccagcc | tgg |
| 25320848 | 1 | caggtgttcaagaccagcct | ggg |
| 25320850 | −1 | cttagccatgttgcccaggc | tgg |
| 25320854 | −1 | aggacttagccatgttgccc | agg |
| 25320856 | 1 | caagaccagcctgggcaaca | tgg |
| 25320874 | −1 | ttggtattttttgcagagac | agg |
| 25320893 | −1 | accatatccagctcagtttt | tgg |
| 25320897 | 1 | aaaaataccaaaaactgagc | tgg |
| 25320903 | 1 | accaaaaactgagctggata | tgg |
| 25320919 | 1 | gatatggtagcacacacctg | tgg |
| 25320924 | −1 | tcccaagtagctgggaccac | agg |
| 25320932 | −1 | cctcagcctcccaagtagct | ggg |
| 25320933 | 1 | cacctgtggtcccagctact | tgg |
| 25320933 | −1 | acctcagcctcccaagtagc | tgg |
| 25320934 | 1 | acctgtggtcccagctactt | ggg |
| 25320937 | 1 | tgtggtcccagctacttggg | ag |
| 25320943 | 1 | cccagctacttgggaggctg | agg |
| 25320946 | 1 | agctacttgggaggctgagg | tgg |
| 25320947 | 1 | gctacttgggaggctgaggt | ggg |
| 25320950 | 1 | acttgggaggctgaggtggg | agg |
| 25320951 | 1 | cttgggaggctgaggtggga | ggg |
| 25320965 | 1 | gtgggagggttgcttgaccc | cgg |
| 25320966 | 1 | tgggagggttgcttgacccc | ggg |
| 25320971 | −1 | attgcagcctcaaactcccg | ggg |
| 25320972 | −1 | cattgcagcctcaaactccc | ggg |
| 25320973 | −1 | tcattgcagcctcaaactcc | cgg |
| 25320975 | 1 | tgcttgaccccgggagtttg | agg |
| 25321008 | −1 | ttatccaggctggagtgcag | tgg |
| 25321015 | 1 | tgtgccactgcactccagcc | tgg |
| 25321018 | −1 | tctcattctgttatccaggc | tgg |
| 25321022 | −1 | agagtctcattctgttatcc | agg |
| 25321047 | −1 | tgattttattttttattttt | ggg |
| 25321048 | −1 | ttgattttattttttatttt | tgg |
| 25321077 | 1 | atcaaagacacttaaaaaga | tgg |
| 25321078 | 1 | tcaaagacacttaaaaagat | ggg |
| 25321079 | 1 | caaagacacttaaaaagatg | ggg |
| 25321085 | 1 | cacttaaaaagatggggaaa | aGG |
| 25321089 | 1 | taaaaagatggggaaaaGGA | AGG |
| 25321094 | 1 | agatggggaaaaGGAAGGAC | AGG |
| 25321132 | −1 | AAGATTCCACTTGTGTAGTT | AGG |
| 25321137 | 1 | TACTTTCCTAACTACACAAG | TGG |
| 25321152 | 1 | ACAAGTGGAATCTTAAGCTG | AGG |
| 25321160 | 1 | AATCTTAAGCTGAGGTTCCC | AGG |
| 25321166 | −1 | TCTGGCTCCAGTCAACTCCT | GGG |
| 25321167 | −1 | CTCTGGCTCCAGTCAACTCC | TGG |
| 25321170 | 1 | TGAGGTTCCCAGGAGTTGAC | TGG |
| 25321184 | −1 | TCCTATAGGTCTGTCTTCTC | TGG |
| 25321194 | 1 | GCCAGAGAAGACAGACCTAT | AGG |
| 25321198 | −1 | CTCCAATTGGGTGCTCCTAT | AGG |
| 25321207 | 1 | GACCTATAGGAGCACCCAAT | TGG |
| 25321210 | −1 | TATGGAGGGTGACTCCAATT | GGG |
| 25321211 | −1 | CTATGGAGGGTGACTCCAAT | TGG |
| 25321224 | −1 | GACATATGGGCTACTATGGA | GGG |
| 25321225 | −1 | AGACATATGGGCTACTATGG | AGG |
| 25321228 | −1 | GTAAGACATATGGGCTACTA | TGG |
| 25321237 | −1 | CTGATCCATGTAAGACATAT | GGG |
| 25321238 | −1 | GCTGATCCATGTAAGACATA | TGG |
| 25321243 | 1 | AGTAGCCCATATGTCTTACA | TGG |
| 25321257 | 1 | CTTACATGGATCAGCTTTCG | TGG |
| 25321258 | 1 | TTACATGGATCAGCTTTCGT | GGG |
| 25321259 | 1 | TACATGGATCAGCTTTCGTG | GGG |
| 25321271 | −1 | CTTCCCCAGATGGAGTAAAA | GGG |
| 25321272 | −1 | CCTTCCCCAGATGGAGTAAA | AGG |
| 25321277 | 1 | TGGGGCCCTTTTACTCCATC | TGG |
| 25321278 | 1 | GGGGCCCTTTTACTCCATCT | GGG |
| 25321279 | 1 | GGGCCCTTTTACTCCATCTG | GGG |
| 25321281 | −1 | ATCTGACGCCCTTCCCCAGA | TGG |
| 25321283 | 1 | CCTTTTACTCCATCTGGGGA | AGG |
| 25321284 | 1 | CTTTTACTCCATCTGGGGAA | GGG |
| 25321298 | 1 | GGGGAAGGGCGTCAGATCTG | TGG |
| 25321335 | −1 | ttGAAAAAAAGAACTGGGAA | TGG |
| 25321340 | −1 | tttttttGAAAAAAAGAACT | GGG |
| 25321341 | −1 | ttttttttGAAAAAAAGAAC | TGG |
| 25321375 | 1 | aaaaaaaaTGTCTACAGAAT | Cgg |
| 25321380 | 1 | aaaTGTCTACAGAATCggcc | agg |
| 25321385 | 1 | TCTACAGAATCggccaggtg | tgg |
| 25321387 | −1 | caggcatgagccaccacacc | tgg |
| 25321388 | 1 | ACAGAATCggccaggtgtgg | tgg |
| 25321406 | −1 | ttccaaagtgctagtattac | agg |
| 25321415 | 1 | tgcctgtaatactagcactt | tgg |
| 25321419 | 1 | tgtaatactagcactttgga | agg |
| 25321425 | 1 | actagcactttggaaggctg | agg |
| 25321428 | 1 | agcactttggaaggctgagg | tgg |
| 25321429 | 1 | gcactttggaaggctgaggt | ggg |
| 25321432 | 1 | ctttggaaggctgaggtggg | tgg |
| 25321443 | 1 | tgaggtgggtggatcacctg | agg |
| 25321447 | 1 | gtgggggatcacctgaggt | cgg |
| 25321448 | 1 | tgggtggatcacctgaggtc | ggg |
| 25321448 | −1 | ggtctcgaactcccgacctc | agg |
| 25321466 | 1 | tcgggagttcgagaccagcc | tgg |
| 25321469 | −1 | tttcaccatgttggccaggc | tgg |
| 25321473 | −1 | ggagtttcaccatgttggcc | agg |
| 25321475 | 1 | cgagaccagcctggccaaca | tgg |
| 25321478 | −1 | gagatggagtttcaccatgt | tgg |
| 25321494 | −1 | ttttttttttttagtagaga | tgg |
| 25321523 | 1 | aaaaaaaaaaaaaaattagc | tgg |
| 25321529 | 1 | aaaaaaaaattagctggatg | tgg |
| 25321532 | 1 | aaaaaattagctggatgtgg | tgg |
| 25321536 | 1 | aattagctggatgtggtggc | agg |
| 25321550 | −1 | tcccaagtagctgagattat | agg |
| 25321559 | 1 | cgcctataatctcagctact | tgg |
| 25321560 | 1 | gcctataatctcagctactt | ggg |
| 25321563 | 1 | tataatctcagctacttggg | agg |
| 25321569 | 1 | ctcagctacttgggaggctg | agg |
| 25321573 | 1 | gctacttgggaggctgaggc | agg |
| 25321591 | 1 | gcaggataatcgcttgaacc | tgg |
| 25321592 | 1 | caggataatcgcttgaacct | ggg |
| 25321595 | 1 | gataatcgcttgaacctggg | agg |
| 25321598 | −1 | cactgcagcctctgcctccc | agg |
| 25321601 | 1 | cgcttgaacctgggaggcag | agg |
| 25321623 | −1 | ggagtacaatggcgtgatct | cgg |
| 25321634 | −1 | tcgcccaggctggagtacaa | tgg |
| 25321641 | 1 | cacgccattgtactccagcc | tgg |
| 25321642 | 1 | acgccattgtactccagcct | ggg |
| 25321644 | −1 | tctcactctatcgcccaggc | tgg |
| 25321648 | −1 | agagtctcactctatcgccc | agg |
| 25321706 | 1 | aaaataaaataaaataaaat | aGG |
| 25321723 | 1 | aataGGCTACAGAATTAAGC | TGG |
| 25321729 | 1 | CTACAGAATTAAGCTGGTCC | AGG |
| 25321736 | −1 | AATGGAAGCCCTGTCATTCC | TGG |
| 25321738 | 1 | TAAGCTGGTCCAGGAATGAC | AGG |
| 25321739 | 1 | AAGCTGGTCCAGGAATGACA | GGG |
| 25321754 | −1 | ACAATTGAAAGACAAATAAA | TGG |
| 25321767 | 1 | ATTTATTTGTCTTTCAATTG | TGG |
| 25321768 | 1 | TTTATTTGTCTTTCAATTGT | GGG |
| 25321777 | 1 | CTTTCAATTGTGGGAGAAAA | AGG |
| 25321851 | −1 | TGTTAAAAGATTTGGAGCAC | AGG |
| 25321859 | −1 | TAATTTAATGTTAAAAGATT | TGG |
| 25321884 | 1 | ATTAAATTATGCATTTAAAC | AGG |
| 25321902 | −1 | CTTTCCATATTTTAAGATTT | AGG |
| 25321909 | 1 | TGCTCCTAAATCTTAAAATA | TGG |
| 25321925 | 1 | AATATGGAAAGCACCTCATG | AGG |
| 25321927 | −1 | TCAAAATATTTAGCCTCATG | AGG |
| 25321953 | −1 | ATCTTACCTTCCAGAAAACT | TGG |
| 25321954 | 1 | ATTTTGATGACCAAGTTTTC | TGG |
| 25321958 | 1 | TGATGACCAAGTTTTCTGGA | AGG |
| 25321982 | −1 | TCAAAATCTATCACGTTAAT | AGG |
| 25322026 | −1 | GCAAGTCAACATATATACTC | AGG |
| 25322067 | 1 | GAGTAAAACAAAAACAAAAA | TGG |
| 25322074 | 1 | ACAAAAACAAAAATGGAGTA | AGG |
| 25322085 | 1 | AATGGAGTAAGGAGCATTGC | AGG |
| 25322088 | 1 | GGAGTAAGGAGCATTGCAGG | AGG |
| 25322097 | 1 | AGCATTGCAGGAGGAACTAG | AGG |
| 25322119 | −1 | CCCCACACACATGCATATCA | TGG |
| 25322128 | 1 | ATCCATGATATGCATGTGTG | TGG |
| 25322129 | 1 | TCCATGATATGCATGTGTGT | GGG |
| 25322130 | 1 | CCATGATATGCATGTGTGTG | GGG |
| 25322131 | 1 | CATGATATGCATGTGTGTGG | GGG |
| 25322134 | 1 | GATATGCATGTGTGTGGGGG | AGG |
| 25322135 | 1 | ATATGCATGTGTGTGGGGGA | GGG |
| 25322138 | 1 | TGCATGTGTGTGGGGGAGGG | TGG |
| 25322141 | 1 | ATGTGTGTGGGGGAGGGTGG | CGG |
| 25322142 | 1 | TGTGTGTGGGGGAGGGTGGC | GGG |
| 25322143 | 1 | GTGTGTGGGGGAGGGTGGCG | GGG |
| 25322146 | 1 | TGTGGGGGAGGGTGGCGGGG | AGG |
| 25322149 | 1 | GGGGGAGGGTGGCGGGGAGG | TGG |
| 25322155 | 1 | GGGTGGGGGGAGGTGGTAA | AGG |
| 25322170 | −1 | AATTTGAGGTATCAGGGAAA | TGG |
| 25322176 | −1 | TGAATGAATTTGAGGTATCA | GGG |
| 25322177 | −1 | CTGAATGAATTTGAGGTATC | AGG |
| 25322184 | −1 | CCTGACTCTGAATGAATTTG | AGG |
| 25322195 | 1 | CCTCAAATTCATTCAGAGTC | AGG |
| 25322196 | 1 | CTCAAATTCATTCAGAGTCA | GGG |
| 25322215 | 1 | AGGGATGAGACAGCTTTCAC | TGG |
| 25322227 | −1 | AGATAGGGGGAGGGGAAGTG | TGG |
| 25322235 | −1 | AGGACTGCAGATAGGGGGAG | GGG |
| 25322236 | −1 | GAGGACTGCAGATAGGGGGA | GGG |
| 25322237 | −1 | TGAGGACTGCAGATAGGGGG | AGG |
| 25322240 | −1 | CGCTGAGGACTGCAGATAGG | GGG |
| 25322241 | −1 | ACGCTGAGGACTGCAGATAG | GGG |
| 25322242 | −1 | TACGCTGAGGACTGCAGATA | GGG |
| 25322243 | −1 | CTACGCTGAGGACTGCAGAT | AGG |
| 25322255 | −1 | CAGACTATTTGGCTACGCTG | AGG |
| 25322266 | −1 | CACCCGCATGTCAGACTATT | TGG |
| 25322274 | 1 | TAGCCAAATAGTCTGACATG | CGG |
| 25322275 | 1 | AGCCAAATAGTCTGACATGC | GGG |
| 25322295 | −1 | cttccagcttttgcattgtg | ggg |
| 25322296 | −1 | tcttccagcttttgcattgt | ggg |
| 25322297 | −1 | ttcttccagcttttgcattg | tgg |
| 25322303 | 1 | gaaccccacaatgcaaaagc | tgg |
| 25322321 | −1 | gggttggactccaaggcttg | agg |
| 25322322 | 1 | ctggaagaaacctcaagcct | tgg |
| 25322328 | −1 | aaaaaaggggttggactcca | agg |
| 25322337 | −1 | gcatctgtcaaaaaaggggt | tgg |
| 25322341 | −1 | cttagcatctgtcaaaaaag | ggg |
| 25322342 | −1 | tcttagcatctgtcaaaaaa | ggg |
| 25322343 | −1 | ctcttagcatctgtcaaaaa | agg |
| 25322357 | 1 | tttttgacagatgctaagag | tgg |
| 25322387 | 1 | acttatcaagatcttacaac | Tgg |
| 25322418 | −1 | tcccaaagtgctgggatcac | agg |
| 25322426 | −1 | cctcagcctcccaaagtgct | ggg |
| 25322427 | 1 | cgcctgtgatcccagcactt | tgg |
| 25322427 | −1 | acctcagcctcccaaagtgc | tgg |
| 25322428 | 1 | gcctgtgatcccagcacttt | ggg |
| 25322431 | 1 | tgtgatcccagcactttggg | agg |
| 25322437 | 1 | cccagcactttgggaggctg | agg |
| 25322440 | 1 | agcactttgggaggctgagg | tgg |
| 25322441 | 1 | gcactttgggaggctgaggt | ggg |
| 25322442 | 1 | cactttgggaggctgaggtg | ggg |
| 25322455 | 1 | tgaggtggggcgatcacctg | agg |
| 25322460 | 1 | tggggcgatcacctgaggcc | agg |
| 25322460 | −1 | ggtctcgaactcctggcctc | agg |
| 25322467 | −1 | ccaggctggtctogaactcc | tgg |
| 25322478 | 1 | ccaggagttcgagaccagcc | tgg |
| 25322481 | −1 | tttcgacacgttggccaggc | tgg |
| 25322485 | −1 | ggggtttcgacacgttggcc | agg |
| 25322490 | −1 | gagatggggtttcgacacgt | tgg |
| 25322504 | −1 | ttgtatttttagtagagatg | ggg |
| 25322505 | −1 | tttgtatttttagtagagat | ggg |
| 25322506 | −1 | ttttgtatttttagtagaga | tgg |
| 25322526 | 1 | ctaaaaatacaaaagttagc | tgg |
| 25322527 | 1 | taaaaatacaaaagttagct | ggs |
| 25322532 | 1 | atacaaaagttagctgggtg | tgg |
| 25322535 | 1 | caaaagttagctgggtgtgg | tgg |
| 25322553 | −1 | tcctgagtaactgggattac | agg |
| 25322561 | −1 | cctcagcctcctgagtaact | ggg |
| 25322562 | −1 | gcctcagcctcctgagtaac | tgg |
| 25322563 | 1 | gcctgtaatcccagttactc | agg |
| 25322566 | 1 | tgtaatcccagttactcagg | agg |
| 25322572 | 1 | cccagttactcaggaggctg | agg |
| 25322576 | 1 | gttactcaggaggctgaggc | agg |
| 25322594 | 1 | gcaggagaatcacttgaacc | tgg |
| 25322595 | 1 | caggagaatcacttgaacct | ggg |
| 25322601 | −1 | cactgcaaacttcgcttccc | agg |
| 25322637 | −1 | tcacccaggctggagtgcag | tgg |
| 25322644 | 1 | catgccactgcactccagcc | tgg |
| 25322645 | 1 | atgccactgcactccagcct | ggg |
| 25322647 | −1 | tctcgctctgtcacccaggc | tgg |
| 25322651 | −1 | aaagtctcgctctgtcaccc | agg |
| 25322675 | −1 | Attgttttgttttgtttttg | agg |
| 25322721 | −1 | gtgtttctctgtaactcact | tgg |
| 25322743 | −1 | cctgaattaggctcaaagtg | tgg |
| 25322754 | 1 | ccacactttgagcctaattc | agg |
| 25322755 | −1 | taataaaggactcctgaatt | agg |
| 25322769 | −1 | tctaggtcgccggctaataa | agg |
| 25322771 | 1 | ttcaggagtcctttattagc | cgg |
| 25322779 | −1 | actagtcgtctctaggtcgc | cgg |
| 25322786 | −1 | tttgagcactagtcgtctct | agg |
| 25322807 | 1 | actagtgctcaaaattctct | cgg |
| 25322819 | 1 | aattctctcggccccaaaga | agg |
| 25322819 | −1 | aaaatctagccccttctttg | ggg |
| 25322820 | 1 | attctctcggccccaaagaa | ggg |
| 25322820 | −1 | gaaaatctagccccttcttt | ggg |
| 25322821 | 1 | ttctctcggccccaaagaag | ggg |
| 25322821 | −1 | agaaaatctagccccttctt | tgg |
| 25322844 | 1 | ctagattttcttttatacct | tgg |
| 25322850 | −1 | ccgctcccctttctaaacca | agg |
| 25322854 | 1 | ttttataccttggtttagaa | agg |
| 25322855 | 1 | tttataccttggtttagaaa | ggg |
| 25322856 | 1 | ttataccttggtttagaaag | ggg |
| 25322861 | 1 | ccttggtttagaaaggggag | cgg |
| 25322862 | 1 | cttggtttagaaaggggagc | ggg |
| 25322898 | 1 | caatcttacagaagtaaaac | agg |
| 25322922 | 1 | aaaaaagttaaaaagacaaa | tgg |
| 25322929 | 1 | ttaaaaagacaaatggttac | agg |
| 25322947 | 1 | acaggaaaacaaacagttcc | agg |
| 25322953 | 1 | aaacaaacagttccaggtgc | agg |
| 25322954 | −1 | ggctttaaagctcctgcacc | tgg |
| 25322974 | 1 | ggagctttaaagccatcaca | agg |
| 25322975 | −1 | ccgcacctgtcaccttgtga | tgg |
| 25322981 | 1 | taaagccatcacaaggtgac | agg |
| 25322986 | 1 | ccatcacaaggtgacaggtg | cgg |
| 25322987 | 1 | catcacaaggtgacaggtgc | ggg |
| 25322988 | 1 | atcacaaggtgacaggtgcg | ggg |
| 25322989 | 1 | tcacaaggtgacaggtgcgg | ggg |
| 25322995 | 1 | ggtgacaggtgcgggggctc | tgg |
| 25322996 | 1 | gtgacaggtgcgggggctct | ggg |
| 25323009 | 1 | gggctctgggtgctatctgc | cgg |
| 25323017 | −1 | agtgcccctgcgtttgtgtc | cgg |
| 25323022 | 1 | tatctgccggacacaaacgc | agg |
| 25323023 | 1 | atctgccggacacaaacgca | ggg |
| 25323024 | 1 | tctgccggacacaaacgcag | ggg |
| 25323045 | 1 | ggcactagagtactatcacc | cgg |
| 25323046 | 1 | gcactagagtactatcaccc | ggg |
| 25323052 | −1 | cagttcccaggaatttgccc | ggg |
| 25323053 | −1 | gcagttcccaggaatttgcc | cgg |
| 25323057 | 1 | ctatcacccgggcaaattcc | tgg |
| 25323058 | 1 | tatcacccgggcaaattcct | ggg |
| 25323064 | −1 | aagctgtgtccgcagttccc | agg |
| 25323066 | 1 | gggcaaattcctgggaactg | cgg |
| 25323088 | −1 | aattagctgataaggtactg | tgg |
| 25323096 | −1 | aagagtgcaattagctgata | agg |
| 25323118 | 1 | aattgcactctttgatgtgc | tgg |
| 25323119 | 1 | attgcactctttgatgtgct | ggg |
| 25323149 | 1 | ttgcacaagttaagtccttg | agg |
| 25323153 | 1 | acaagttaagtccttgagga | agg |
| 25323153 | −1 | cttacccacccccttcctca | agg |
| 25323154 | 1 | caagttaagtccttgaggaa | ggg |
| 25323155 | 1 | aagttaagtccttgaggaag | ggg |
| 25323156 | 1 | agttaagtccttgaggaagg | ggg |
| 25323159 | 1 | taagtccttgaggaaggggg | tgg |
| 25323160 | 1 | aagtccttgaggaagggggt | ggg |
| 25323165 | 1 | cttgaggaagggggtgggta | agg |
| 25323179 | −1 | cttcatttgcaagacgttaa | ggg |
| 25323180 | −1 | ccttcatttgcaagacgtta | agg |
| 25323191 | 1 | ccttaacgtcttgcaaatga | agg |
| 25323201 | 1 | ttgcaaatgaaggagccgaa | tgg |
| 25323205 | −1 | aaagccggagggattccatt | cgg |
| 25323212 | 1 | ggagccgaatggaatccctc | cgg |
| 25323216 | −1 | tcttagctaagaaagccgga | ggg |
| 25323217 | −1 | ctcttagctaagaaagccgg | agg |
| 25323220 | −1 | tctctcttagctaagaaagc | cgg |
| 25323256 | 1 | caatcaagttaatacaagtt | agg |
| 25323257 | 1 | aatcaagttaatacaagtta | ggg |
| 25323323 | −1 | ccttgtcttgatggtggtga | tgg |
| 25323329 | −1 | tgtgctccttgtcttgatgg | tgg |
| 25323332 | −1 | gggtgtgctccttgtcttga | tgg |
| 25323334 | 1 | ccatcaccaccatcaagaca | agg |
| 25323352 | −1 | aggaagtgtgtggaagtgat | ggg |
| 25323353 | −1 | gaggaagtgtgtggaagtga | tgg |
| 25323362 | −1 | aaggagcaggaggaagtgtg | tgg |
| 25323372 | −1 | aggaatttcaaaggagcagg | agg |
| 25323375 | −1 | gggaggaatttcaaaggagc | agg |
| 25323381 | −1 | tagggagggaggaatttcaa | agg |
| 25323392 | −1 | gaccaggtgggtagggaggg | agg |
| 25323395 | −1 | tgggaccaggtgggtaggga | ggg |
| 25323396 | −1 | gtgggaccaggtgggtaggg | agg |
| 25323399 | −1 | tgggtgggaccaggtgggta | ggg |
| 25323400 | −1 | ttgggtgggaccaggtgggt | agg |
| 25323401 | 1 | ttcctccctccctacccacc | tgg |
| 25323404 | −1 | gcctttgggtgggaccaggt | ggg |
| 25323405 | −1 | tgcctttgggtgggaccagg | tgg |
| 25323408 | −1 | ggttgcctttgggtgggacc | agg |
| 25323414 | 1 | acccacctggtcccacccaa | agg |
| 25323414 | −1 | ttcagtggttgcctttgggt | ggg |
| 25323415 | −1 | gttcagtggttgcctttggg | tgg |
| 25323418 | −1 | gtagttcagtggttgccttt | ggg |
| 25323419 | −1 | agtagttcagtggttgcctt | tgg |
| 25323429 | −1 | agtgacagaaagtagttcag | tgg |
| 25323444 | 1 | tgaactactttctgtcacta | agg |
| 25323479 | 1 | gtaatttttttgtttgagac | agg |
| 25323480 | 1 | taatttttttgtttgagaca | ggg |
| 25323499 | −1 | ctgcattacggtgtgggtgg | cgg |
| 25323502 | −1 | ccactgcattacggtgtggg | tgg |
| 25323505 | −1 | gtgccactgcattacggtgt | ggg |
| 25323506 | −1 | ggtgccactgcattacggtg | tgg |
| 25323511 | −1 | atgatggtgccactgcatta | cgg |
| 25323513 | 1 | ccacccacaccgtaatgcag | tgg |
| 25323524 | 1 | gtaatgcagtggcaccatca | tgg |
| 25323527 | −1 | gaggctacagtgagccatga | tgg |
| 25323546 | −1 | tcctgagcctggggaggttg | agg |
| 25323550 | 1 | actgtagcctcaacctcccc | agg |
| 25323552 | −1 | aggatctcctgagcctgggg | agg |
| 25323555 | −1 | gggaggatctcctgagcctg | ggg |
| 25323556 | 1 | gcctcaacctccccaggctc | agg |
| 25323556 | −1 | ggggaggatctcctgagcct | ggg |
| 25323557 | −1 | gggggaggatctcctgagcc | tgg |
| 25323572 | −1 | actcaggaggctgagggggg | agg |
| 25323575 | −1 | gctactcaggaggctgaggg | ggg |
| 25323576 | −1 | agctactcaggaggctgagg | ggg |
| 25323577 | −1 | tagctactcaggaggctgag | ggg |
| 25323578 | −1 | ctagctactcaggaggctga | ggg |
| 25323579 | −1 | cctagctactcaggaggctg | ggg |
| 25323585 | −1 | tgtggtcctagctactcagg | agg |
| 25323588 | −1 | acctgtggtcctagctactc | agg |
| 25323590 | 1 | cctcagcctcctgagtagct | agg |
| 25323598 | 1 | tcctgagtagctaggaccac | agg |
| 25323603 | −1 | gccatggtggcctacacctg | tgg |
| 25323604 | 1 | gtagctaggaccacaggtgt | agg |
| 25323613 | 1 | accacaggtgtaggccacca | tgg |
| 25323616 | −1 | caaaaattagcctgccatgg | tgg |
| 25323617 | 1 | caggtgtaggccaccatggc | agg |
| 25323619 | −1 | atacaaaaattagcctgcca | tgg |
| 25323646 | 1 | tttgtatttttttgtagaga | tgg |
| 25323647 | 1 | ttgtatttttttgtagagat | ggg |
| 25323648 | 1 | tgtatttttttgtagagatg | ggg |
| 25323665 | −1 | cgagaccagcctaggtaata | cgg |
| 25323667 | 1 | ggggtttcaccgtattacct | agg |
| 25323671 | 1 | tttcaccgtattacctaggc | tgg |
| 25323673 | 1 | catgagttcgagaccagcct | agg |
| 25323685 | 1 | ctaggctggtctcgaactca | tgg |
| 25323686 | 1 | taggctggtctcgaactcat | ggg |
| 25323708 | −1 | ctttgagaggccaaggcagg | agg |
| 25323709 | 1 | ttcaagcaatcctcctgcct | tgg |
| 25323711 | −1 | gcactttgagaggccaaggc | agg |
| 25323715 | −1 | cccagcactttgagaggcca | agg |
| 25323721 | −1 | tataatcccagcactttgag | agg |
| 25323725 | 1 | gccttggcctctcaaagtgc | tgg |
| 25323726 | 1 | ccttggcctctcaaagtgct | ggg |
| 25323734 | 1 | tctcaaagtgctgggattat | agg |
| 25323752 | −1 | ttacagagggctgggcacag | tgg |
| 25323760 | −1 | gtgtaacattacagagggct | ggg |
| 25323761 | −1 | tgtgtaacattacagagggc | tgg |
| 25323765 | −1 | cctttgtgtaacattacaga | ggg |
| 25323766 | −1 | ccctttgtgtaacattacag | agg |
| 25323776 | 1 | ccctctgtaatgttacacaa | agg |
| 25323777 | 1 | cctctgtaatgttacacaaa | ggg |
| 25323803 | 1 | catgcagcacgtactgccct | tgg |
| 25323808 | 1 | agcacgtactgcccttggtc | tgg |
| 25323808 | −1 | agcaaaagaagccagaccaa | ggg |
| 25323809 | −1 | gagcaaaagaagccagacca | agg |
| 25323855 | −1 | gtcagttacacgcaacaaca | cgg |
| 25323901 | 1 | tctctgcAGCTGTCAGCTCT | TGG |
| 25323918 | −1 | ATAAAGAGAGATTGGCTGTT | GGG |
| 25323919 | −1 | GATAAAGAGAGATTGGCTGT | TGG |
| 25323926 | −1 | TGCAGGGGATAAAGAGAGAT | TGG |
| 25323941 | −1 | ATAGGCAAGAACACTTGCAG | GGG |
| 25323942 | −1 | AATAGGCAAGAACACTTGCA | GGG |
| 25323943 | −1 | AAATAGGCAAGAACACTTGC | AGG |
| 25323959 | −1 | GTACCTTGATTCTGCTAAAT | AGG |
| 25323967 | 1 | TTGCCTATTTAGCAGAATCA | AGG |
| 25323988 | 1 | GGTACTCTATCGAAAAGACT | CGG |
| 25323996 | 1 | ATCGAAAAGACTCGGAAAAT | TGG |
| 25324022 | 1 | AATCTattcattcattcctc | agg |
| 25324027 | −1 | agttattcgataaatacctg | agg |
| 25324058 | −1 | tggttgattagcatagtact | tgg |
| 25324072 | 1 | agtactatgctaatcaacca | agg |
| 25324078 | −1 | tctcctgtttgtgctgtcct | tgg |
| 25324086 | 1 | caaccaaggacagcacaaac | agg |
| 25324106 | −1 | TGCAACTCAAGTGACTGAGC | TGg |
| 25324132 | 1 | GAGTTGCAATAAATATTTGC | TGG |
| 25324137 | 1 | GCAATAAATATTTGCTGGAT | AGg |
| 25324142 | 1 | AAATATTTGCTGGATAGgtc | agg |
| 25324150 | 1 | GCTGGATAGgtcaggtgcag | tgg |
| 25324176 | −1 | tcagtaatccccaaagtgct | ggg |
| 25324177 | 1 | cacttgtaatcccagcactt | tgg |
| 25324177 | −1 | ctcagtaatccccaaagtgc | tgg |
| 25324178 | 1 | acttgtaatcccagcacttt | ggg |
| 25324179 | 1 | cttgtaatcccagcactttg | ggg |
| 25324192 | 1 | cactttggggattactgaga | cgg |
| 25324193 | 1 | actttggggattactgagac | ggg |
| 25324196 | 1 | ttggggattactgagacggg | agg |
| 25324212 | 1 | cgggaggatctcttgagccc | agg |
| 25324215 | 1 | gaggatctcttgagcccagg | agg |
| 25324218 | −1 | ctctgcagccttggcctect | ggg |
| 25324219 | −1 | tctctgcagccttggcctcc | tgg |
| 25324221 | 1 | ctcttgagcccaggaggcca | agg |
| 25324227 | −1 | atcatggttctctgcagcct | tgg |
| 25324243 | −1 | ggagtgcagtggcatgatca | tgg |
| 25324254 | −1 | tcacccaggctggagtgcag | tgg |
| 25324261 | 1 | catgccactgcactccagcc | tgg |
| 25324262 | 1 | atgccactgcactccagcct | ggg |
| 25324264 | −1 | tctcactctgtcacccaggc | tgg |
| 25324268 | −1 | aggatctcactctgtcaccc | agg |
| 25324288 | −1 | AAATAttttttttcagagac | agg |
| 25324304 | 1 | ctctgaaaaaaaaTATTTGC | TGG |
| 25324315 | 1 | aaTATTTGCTGGATAAATTA | AGG |
| 25324339 | −1 | TGCTGCAATGGCTACTGATG | GGG |
| 25324340 | −1 | TTGCTGCAATGGCTACTGAT | GGG |
| 25324341 | −1 | GTTGCTGCAATGGCTACTGA | TGG |
| 25324351 | −1 | TAGTTTACCTGTTGCTGCAA | TGG |
| 25324355 | 1 | TCAGTAGCCATTGCAGCAAC | AGG |
| 25324380 | 1 | AACTAGAACGAGTGTGAATT | TGG |
| 25324387 | 1 | ACGAGTGTGAATTTGGAATG | AGG |
| 25324401 | 1 | GGAATGAGGAAACCCGATGT | TGG |
| 25324402 | −1 | ACAGAATGATGGCCAACATC | GGG |
| 25324403 | −1 | TACAGAATGATGGCCAACAT | CGG |
| 25324413 | −1 | tacatgacatTACAGAATGA | TGG |
| 25324464 | 1 | tattaatgtatgtattatgt | agg |
| 25324482 | −1 | gttaccagtgagagaggtca | agg |
| 25324488 | −1 | tcttatgttaccagtgagag | agg |
| 25324489 | 1 | agttccttgacctctctcac | tgg |
| 25324526 | 1 | taatctttgtgctacttcac | tgg |
| 25324527 | 1 | aatctttgtgctacttcact | ggg |
| 25324549 | 1 | gttattttaaagatcaagtg | agg |
| 25324597 | −1 | aaactttcacattcatgtgg | cgg |
| 25324600 | −1 | aataaactttcacattcatg | tgg |
| 25324617 | 1 | gaatgtgaaagtttattact | aGG |
| 25324618 | 1 | aatgtgaaagtttattacta | GGG |
| 25324636 | 1 | taGGGATTTAGCCAACCACA | AGG |
| 25324636 | −1 | CTCACACATTCCCTTGTGGT | TGG |
| 25324637 | 1 | aGGGATTTAGCCAACCACAA | GGG |
| 25324640 | −1 | TATGCTCACACATTCCCTTG | TGG |
| 25324682 | −1 | agcacaaaatcagaaactgt | agg |
| 25324696 | 1 | acagtttctgattttgtgct | agg |
| 25324715 | −1 | gaggataaaatcaggtaatg | tgg |
| 25324723 | −1 | gctgttgtgaggataaaatc | agg |
| 25324734 | −1 | ttttatgcagggctgttgtg | agg |
| 25324745 | −1 | gacatacttacttttatgca | ggg |
| 25324746 | −1 | cgacatacttacttttatgc | agg |
| 25324763 | 1 | taaaagtaagtatgtcgccc | agg |
| 25324768 | 1 | gtaagtatgtcgcccaggtg | cgg |
| 25324769 | −1 | aggcatgagccaccgcacct | ggg |
| 25324770 | −1 | taggcatgagccaccgcacc | tgg |
| 25324771 | 1 | agtatgtcgcccaggtgcgg | tgg |
| 25324789 | −1 | tcccaaagtgctgggattat | agg |
| 25324797 | −1 | cctcgggctcccaaagtgct | ggg |
| 25324798 | 1 | tgcctataatcccagcactt | tgg |
| 25324798 | −1 | acctcgggctcccaaagtgc | tgg |
| 25324799 | 1 | gcctataatcccagcacttt | ggg |
| 25324808 | 1 | cccagcactttgggagcccg | agg |
| 25324811 | 1 | agcactttgggagcccgagg | tgg |
| 25324812 | 1 | gcactttgggagcccgaggt | ggg |
| 25324813 | −1 | tcaagtgatttgcccacctc | ggg |
| 25324814 | −1 | ctcaagtgatttgcccacct | cgg |
| 25324831 | 1 | tgggcaaatcacttgagatc | agg |
| 25324849 | 1 | tcaggagtttgaaaccagcc | tgg |
| 25324852 | −1 | ttgcaccacgttgaccaggc | tgg |
| 25324856 | −1 | agggttgcaccacgttgacc | agg |
| 25324858 | 1 | tgaaaccagcctggtcaacg | tgg |
| 25324875 | −1 | ttgtatttttagtagagaca | ggg |
| 25324876 | −1 | tttgtatttttagtagagac | agg |
| 25324902 | 1 | aatacaaaaaaaaattagac | agg |
| 25324907 | 1 | aaaaaaaaattagacaggcg | tgg |
| 25324910 | 1 | aaaaaattagacaggcgtgg | tgg |
| 25324913 | 1 | aaattagacaggcgtggtgg | tgg |
| 25324928 | −1 | tcccaagtagctgggattac | agg |
| 25324936 | −1 | cctcagcttcccaagtagct | ggg |
| 25324937 | 1 | tgcctgtaatcccagctact | tgg |
| 25324937 | −1 | gcctcagcttcccaagtagc | tgg |
| 25324938 | 1 | gcctgtaatcccagctactt | ggg |
| 25324947 | 1 | cccagctacttgggaagctg | agg |
| 25324951 | 1 | gctacttgggaagctgaggc | agg |
| 25324958 | 1 | gggaagctgaggcaggagaa | tgg |
| 25324969 | 1 | gcaggagaatggcttgagcc | cgg |
| 25324970 | 1 | caggagaatggcttgagccc | ggg |
| 25324976 | 1 | aatggcttgagcccgggaga | tgg |
| 25324976 | −1 | cactgcaatctccatctccc | ggg |
| 25324977 | −1 | tcactgcaatctccatctcc | cgg |
| 25325012 | −1 | tcacccaggctggagtgcag | tgg |
| 25325019 | 1 | tgcgccactgcactccagcc | tgg |
| 25325020 | 1 | gcgccactgcactccagcct | ggg |
| 25325022 | −1 | ccttgctctgtcacccaggc | tgg |
| 25325026 | −1 | atagccttgctctgtcaccc | agg |
| 25325033 | 1 | ccagcctgggtgacagagca | agg |
| 25325091 | 1 | cagtcttgaagatgatgaaa | tgg |
| 25325094 | 1 | tcttgaagatgatgaaatgg | agg |
| 25325106 | −1 | gcaagttacttaatctctct | agg |
| 25325128 | −1 | tgcattagttctgtcatttt | ggg |
| 25325129 | −1 | atgcattagttctgtcattt | tgg |
| 25325168 | 1 | agaagaaatgtgatgtcttt | tgg |
| 25325182 | −1 | ACGCATATGTGGGGTGTctt | tgg |
| 25325191 | −1 | CTGTAACCAACGCATATGTG | GGG |
| 25325192 | −1 | ACTGTAACCAACGCATATGT | GGG |
| 25325193 | −1 | AACTGTAACCAACGCATATG | TGG |
| 25325196 | 1 | aagACACCCCACATATGCGT | TGG |
| 25325233 | −1 | TTCtgggggtggggtggggg | tgg |
| 25325236 | −1 | GATTTCtggggtgggggtgg | ggg |
| 25325237 | −1 | AGATTTCtggggtgggggtg | ggg |
| 25325238 | −1 | AAGATTTCtgggggtggggt | ggg |
| 25325239 | −1 | GAAGATTTCtgggggtgggg | tgg |
| 25325242 | −1 | TCAGAAGATTTCtgggggtg | ggg |
| 25325243 | −1 | GTCAGAAGATTTCtgggggt | ggg |
| 25325244 | −1 | AGTCAGAAGATTTCtggggg | tgg |
| 25325247 | −1 | ACAAGTCAGAAGATTTCtgg | ggg |
| 25325248 | −1 | AACAAGTCAGAAGATTTCtg | ggg |
| 25325249 | −1 | AAACAAGTCAGAAGATTTCt | ggg |
| 25325250 | −1 | AAAACAAGTCAGAAGATTTC | tgg |
| 25325277 | 1 | TTGTTTTCTCGCAGTTGAGT | AGG |
| 25325290 | 1 | GTTGAGTAGGACCATTTATT | CGG |
| 25325290 | −1 | ATGGTACACTGCCGAATAAA | TGG |
| 25325309 | −1 | TTTCAACTGCAAGCTGAGAA | TGG |
| 25325333 | −1 | TTGCCTCTTTAATGGATATT | TGG |
| 25325341 | 1 | AAGCCAAATATCCATTAAAG | AGG |
| 25325341 | −1 | TTGCATCCTTGCCTCTTTAA | TGG |
| 25325346 | 1 | AAATATCCATTAAAGAGGCA | AGG |
| 25325376 | 1 | CTTGCTAAGCTGATAAATCC | AGG |
| 25325377 | 1 | TTGCTAAGCTGATAAATCCA | GGG |
| 25325378 | 1 | TGCTAAGCTGATAAATCCAG | GGG |
| 25325383 | −1 | aaaaaaaaaaaaaTCACCCC | TGG |
| 25325412 | −1 | ATTTAAAATGTCTTGTTGGA | TGG |
| 25325416 | −1 | GAGTATTTAAAATGTCTTGT | TGG |
| 25325455 | 1 | ATTTCATAGAACTGACTGCC | AGG |
| 25325460 | 1 | ATAGAACTGACTGCCAGGAT | TGG |
| 25325462 | −1 | CTTTAATGTCTTTCCAATCC | TGG |
| 25325485 | −1 | CAGCGAGGCAGTGGCTGAGC | TGG |
| 25325494 | −1 | CTGGCCAACCAGCGAGGCAG | TGG |
| 25325497 | 1 | CAGCTCAGCCACTGCCTCGC | TGG |
| 25325500 | −1 | CGTGGTCTGGCCAACCAGCG | AGG |
| 25325501 | 1 | TCAGCCACTGCCTCGCTGGT | TGG |
| 25325513 | −1 | CAGAAGTGCCAGGCGTGGTC | TGG |
| 25325516 | 1 | CTGGTTGGCCAGACCACGCC | TGG |
| 25325518 | −1 | CCTCCCAGAAGTGCCAGGCG | TGG |
| 25325523 | −1 | TGCTCCCTCCCAGAAGTGCC | AGG |
| 25325525 | 1 | CAGACCACGCCTGGCACTTC | TGG |
| 25325526 | 1 | AGACCACGCCTGGCACTTCT | GGG |
| 25325529 | 1 | CCACGCCTGGCACTTCTGGG | AGG |
| 25325530 | 1 | CACGCCTGGCACTTCTGGGA | GGG |
| 25325550 | −1 | AGATGGGTGCCCTTGGGGGG | TGG |
| 25325551 | 1 | GGAGCACTCACCACCCCCCA | AGG |
| 25325552 | 1 | GAGCACTCACCACCCCCCAA | GGG |
| 25325553 | −1 | ATGAGATGGGTGCCCTTGGG | GGG |
| 25325554 | −1 | GATGAGATGGGTGCCCTTGG | GGG |
| 25325555 | −1 | GGATGAGATGGGTGCCCTTG | GGG |
| 25325556 | −1 | AGGATGAGATGGGTGCCCTT | GGG |
| 25325557 | −1 | GAGGATGAGATGGGTGCCCT | TGG |
| 25325566 | −1 | AAACCTTCGGAGGATGAGAT | GGG |
| 25325567 | −1 | TAAACCTTCGGAGGATGAGA | TGG |
| 25325574 | 1 | GCACCCATCTCATCCTCCGA | AGG |
| 25325576 | −1 | GCATTTTCATAAACCTTCGG | AGG |
| 25325579 | −1 | AGTGCATTTTCATAAACCTT | CGG |
| 25325621 | −1 | AAATTAGGTAATACACGTAG | TGG |
| 25325636 | −1 | TTCACATCGTGTCACAAATT | AGG |
| 25325662 | −1 | TTTATTTAGAATTATCTCTC | TGG |
| 25325685 | 1 | TTCTAAATAAAATATAGTTA | TGG |
| 25325686 | 1 | TCTAAATAAAATATAGTTAT | GGG |
| 25325694 | 1 | AAATATAGTTATGGGTCTCA | AGG |
| 25325708 | −1 | GGATAGGAGATTAGCATATC | TGG |
| 25325724 | −1 | ACTGTAAACTGCAGGAGGAT | AGG |
| 25325729 | −1 | GGACCACTGTAAACTGCAGG | AGG |
| 25325732 | −1 | TGAGGACCACTGTAAACTGC | AGG |
| 25325737 | 1 | TATCCTCCTGCAGTTTACAG | TGG |
| 25325750 | −1 | TTGTAAATAAGTATCTGGTG | AGG |
| 25325755 | −1 | AATTTTTGTAAATAAGTATC | TGG |
| 25325815 | 1 | agagtcttgctctatagctc | agg |
| 25325829 | 1 | tagctcaggctagagtgtaa | tgg |
| 25325840 | 1 | agagtgtaatggtgtgatct | cgg |
| 25325862 | −1 | cacttgaacctgggaggcag | agg |
| 25325865 | 1 | cacttcaacctctgcctccc | agg |
| 25325868 | −1 | gagaatcacttgaacctggg | agg |
| 25325871 | −1 | caggagaatcacttgaacct | ggg |
| 25325872 | −1 | gcaggagaatcacttgaacc | tgg |
| 25325890 | −1 | gctacttgggaggttgaggc | agg |
| 25325894 | −1 | cccagctacttgggaggttg | agg |
| 25325900 | −1 | tgtagtcccagctacttggg | agg |
| 25325903 | −1 | gcctgtagtcccagctactt | ggg |
| 25325904 | 1 | gcctcaacctcccaagtagc | tgg |
| 25325904 | −1 | tgcctgtagtcccagctact | tgg |
| 25325905 | 1 | cctcaacctcccaagtagct | ggg |
| 25325913 | 1 | tcccaagtagctgggactac | agg |
| 25325927 | −1 | caaaaattagccgtggtggc | agg |
| 25325928 | 1 | actacaggcacctgccacca | cgg |
| 25325931 | −1 | actccaaaaattagccgtgg | tgg |
| 25325934 | −1 | aaaactccaaaaattagccg | tgg |
| 25325939 | 1 | ctgccaccacggctaatttt | tgg |
| 25325957 | 1 | tttggagttttagtagagac | agg |
| 25325958 | 1 | ttggagttttagtagagaca | ggg |
| 25325972 | 1 | gagacagggtttcaccacgt | tgg |
| 25325975 | −1 | cgaggccagcctggccaacg | tgg |
| 25325977 | 1 | agggtttcaccacgttggcc | agg |
| 25325981 | 1 | tttcaccacgttggccaggc | tgg |
| 25325984 | −1 | tcaggagttcgaggccagcc | tgg |
| 25325993 | −1 | cacctgaggtcaggagttcg | agg |
| 25326002 | 1 | ggcctcgaactcctgacctc | agg |
| 25326002 | −1 | tgggcagatcacctgaggtc | agg |
| 25326007 | −1 | tgatgtgggcagatcacctg | agg |
| 25326021 | −1 | acattttgggaggctgatgt | ggg |
| 25326022 | −1 | aacattttgggaggctgatg | tgg |
| 25326031 | −1 | tgtaatcccaacattttggg | agg |
| 25326034 | −1 | gcctgtaatcccaacatttt | ggg |
| 25326035 | 1 | acatcagcctcccaaaatgt | tgg |
| 25326035 | −1 | cgcctgtaatcccaacattt | tgg |
| 25326036 | 1 | catcagcctcccaaaatgtt | ggg |
| 25326044 | 1 | tcccaaaatgttgggattac | agg |
| 25326062 | −1 | GAAGTTTTggccgggcatgg | tgg |
| 25326063 | 1 | caggcgtgagccaccatgcc | cgg |
| 25326065 | −1 | ACTGAAGTTTTggccgggca | tgg |
| 25326070 | −1 | TATAAACTGAAGTTTTggcc | ggg |
| 25326071 | −1 | TTATAAACTGAAGTTTTggc | cgg |
| 25326075 | −1 | TGTGTTATAAACTGAAGTTT | Tgg |
| 25326141 | −1 | TATTAAACTGAAATAAAGAA | GGG |
| 25326142 | −1 | TTATTAAACTGAAATAAAGA | AGG |
| 25326160 | 1 | TATTTCAGTTTAATAAACCA | TGG |
| 25326166 | −1 | AAAGCATGAAATAAAATCCA | TGG |
| 25326190 | 1 | TTCATGCTTTGCAAAACACA | AGG |
| 25326191 | 1 | TCATGCTTTGCAAAACACAA | GGG |
| 25326224 | 1 | TGCACTTCTTAAACTAATTC | TGG |
| 25326228 | 1 | CTTCTTAAACTAATTCTGGC | TGG |
| 25326243 | −1 | tcccaaagtgctggaattac | agg |
| 25326252 | 1 | cgcctgtaattccagcactt | tgg |
| 25326252 | −1 | gcctcagcctoccaaagtgc | tgg |
| 25326253 | 1 | gcctgtaattccagcacttt | ggg |
| 25326256 | 1 | tgtaattccagcactttggg | agg |
| 25326262 | 1 | tccagcactttgggaggctg | agg |
| 25326274 | −1 | cctgacttgaagtgatctgt | cgg |
| 25326285 | 1 | ccgacagatcacttcaagtc | agg |
| 25326303 | 1 | tcaggagttcaagaccagcc | tgg |
| 25326306 | −1 | tttcaccatattggccaggc | tgg |
| 25326310 | −1 | gtggtttcaccatattggcc | agg |
| 25326312 | 1 | caagaccagcctggccaata | tgg |
| 25326315 | −1 | gagacgtggtttcaccatat | tgg |
| 25326329 | −1 | ttatatttttggtagagacg | tgg |
| 25326340 | −1 | tggctaattttttatatttt | tgg |
| 25326353 | 1 | aaaaatataaaaaattagcc | agg |
| 25326358 | 1 | tataaaaaattagccaggtg | tgg |
| 25326360 | −1 | tagtcacgcaccaccacacc | tgg |
| 25326361 | 1 | aaaaaattagccaggtgtgg | tgg |
| 25326387 | −1 | cctcaggcccctgagtagct | ggg |
| 25326388 | −1 | gcctcaggcccctgagtagc | tgg |
| 25326389 | 1 | gactataatcccagctactc | agg |
| 25326390 | 1 | actataatcccagctactca | ggg |
| 25326391 | 1 | ctataatcccagctactcag | ggg |
| 25326398 | 1 | cccagctactcaggggcctg | agg |
| 25326403 | −1 | tcaagtgatttttctgcctc | agg |
| 25326420 | 1 | gcagaaaaatcacttgaacc | cgg |
| 25326421 | 1 | cagaaaaatcacttgaaccc | ggg |
| 25326424 | 1 | aaaaatcacttgaacccggg | agg |
| 25326427 | 1 | aatcacttgaacccgggagg | cgg |
| 25326427 | −1 | cactgtaacctccgcctccc | ggg |
| 25326428 | −1 | tcactgtaacctccgcctcc | cgg |
| 25326430 | 1 | cacttgaacccgggaggcgg | agg |
| 25326463 | −1 | tcgcccaggctggagtgcag | tgg |
| 25326470 | 1 | cgcgccactgcactccagcc | tgg |
| 25326471 | 1 | gcgccactgcactccagcct | ggg |
| 25326473 | −1 | tctcactctgtcgcccaggc | tgg |
| 25326477 | −1 | agagtctcactctgtcgccc | agg |
| 25326543 | 1 | aaataCGAAACAAGCAATCC | TGG |
| 25326550 | −1 | TCATTCCAGCAGCTACTGCC | AGG |
| 25326556 | 1 | GCAATCCTGGCAGTAGCTGC | TGG |
| 25326565 | 1 | GCAGTAGCTGCTGGAATGAG | AGG |
| 25326568 | 1 | GTAGCTGCTGGAATGAGAGG | AGG |
| 25326569 | 1 | TAGCTGCTGGAATGAGAGGA | GGG |
| 25326574 | 1 | GCTGGAATGAGAGGAGGGAG | AGG |
| 25326581 | 1 | TGAGAGGAGGGAGAGGTCAT | AGG |
| 25326582 | 1 | GAGAGGAGGGAGAGGTCATA | GGG |
| 25326585 | 1 | AGGAGGGAGAGGTCATAGGG | AGG |
| 25326589 | 1 | GGGAGAGGTCATAGGGAGGT | CGG |
| 25326590 | 1 | GGAGAGGTCATAGGGAGGTC | GGG |
| 25326591 | 1 | GAGAGGTCATAGGGAGGTCG | GGG |
| 25326598 | 1 | CATAGGGAGGTCGGGGACAA | TGG |
| 25326605 | 1 | AGGTCGGGGACAATGGAGCA | TGG |
| 25326616 | 1 | AATGGAGCATGGAGTTGTGT | TGG |
| 25326622 | 1 | GCATGGAGTTGTGTTGGATT | TGG |
| 25326634 | 1 | GTTGGATTTGGCTAAGCAGC | AGG |
| 25326644 | 1 | GCTAAGCAGCAGGAAGTGCA | AGG |
| 25326660 | 1 | TGCAAGGCATTCCAAGCAAG | AGG |
| 25326660 | −1 | CCTGCCCCCCTCCTCTTGCT | TGG |
| 25326663 | 1 | AAGGCATTCCAAGCAAGAGG | AGG |
| 25326664 | 1 | AGGCATTCCAAGCAAGAGGA | GGG |
| 25326665 | 1 | GGCATTCCAAGCAAGAGGAG | GGG |
| 25326666 | 1 | GCATTCCAAGCAAGAGGAGG | GGG |
| 25326667 | 1 | CATTCCAAGCAAGAGGAGGG | GGG |
| 25326671 | 1 | CCAAGCAAGAGGAGGGGGGC | AGG |
| 25326674 | 1 | AGCAAGAGGAGGGGGGCAGG | TGG |
| 25326675 | 1 | GCAAGAGGAGGGGGGCAGGT | GGG |
| 25326676 | 1 | CAAGAGGAGGGGGGCAGGTG | GGG |
| 25326713 | 1 | CAGAAGCAGCATGAGCAACC | TGG |
| 25326718 | 1 | GCAGCATGAGCAACCTGGCT | CGG |
| 25326720 | −1 | TTTTCACACACTGCCGAGCC | AGG |
| 25326733 | 1 | TGGCTCGGCAGTGTGTGAAA | AGG |
| 25326741 | 1 | CAGTGTGTGAAAAGGCTGAA | AGG |
| 25326744 | 1 | TGTGTGAAAAGGCTGAAAGG | TGG |
| 25326762 | −1 | CCTGAAGGATGAAATTGAAG | TGG |
| 25326773 | 1 | CCACTTCAATTTCATCCTTC | AGG |
| 25326777 | −1 | GGAATTTCCCATTTGCCTGA | AGG |
| 25326780 | 1 | AATTTCATCCTTCAGGCAAA | TGG |
| 25326781 | 1 | ATTTCATCCTTCAGGCAAAT | GGG |
| 25326794 | 1 | GGCAAATGGGAAATTCCCAA | AGG |
| 25326798 | −1 | GCTTCCCCACTCAAACCTTT | GGG |
| 25326799 | −1 | TGCTTCCCCACTCAAACCTT | TGG |
| 25326803 | 1 | GAAATTCCCAAAGGTTTGAG | TGG |
| 25326804 | 1 | AAATTCCCAAAGGTTTGAGT | GGG |
| 25326805 | 1 | AATTCCCAAAGGTTTGAGTG | GGG |
| 25326825 | −1 | CACTCTCAAACTTTCATTGT | AGG |
| 25326865 | 1 | AGTGATCGAATTAAGCATGT | AGG |
| 25326877 | −1 | ATTGCAGTTATTTCAGAACT | CGG |
| 25326909 | 1 | ATGTGCTGAAGATCATCCAT | TGG |
| 25326914 | −1 | AATACTCATTCAGAAGCCAA | TGG |
| 25326967 | −1 | ACAGTAGTGTTTATCTTTCT | TGG |
| 25326983 | 1 | AAAGATAAACACTACTGTTT | TGG |
| 25327023 | −1 | CTTCGCGTAAAACAGCAAGA | GGG |
| 25327024 | −1 | ACTTCGCGTAAAACAGCAAG | AGG |
| 25327062 | 1 | AAAATCTACTCTTGTCACAG | TGG |
| 25327079 | −1 | TTATTTGAAATCAGAAGTAG | GGG |
| 25327080 | −1 | TTTATTTGAAATCAGAAGTA | GGG |
| 25327081 | −1 | ATTTATTTGAAATCAGAAGT | AGG |
| 25327112 | 1 | AATGTTCTAGAGACACAGTA | AGG |
| 25327113 | 1 | ATGTTCTAGAGACACAGTAA | GGG |
| 25327125 | −1 | TTGTTGAACAAGCGTTTGTT | GGG |
| 25327126 | −1 | GTTGTTGAACAAGCGTTTGT | TGG |
| 25327143 | 1 | AACGCTTGTTCAACAACACA | AGG |
| 25327159 | −1 | TGTTTTCCTACTTTAAAAGC | TGG |
| 25327164 | 1 | GGAGAGCCAGCTTTTAAAGT | AGG |
| 25327172 | 1 | AGCTTTTAAAGTAGGAAAAC | Agg |
| 25327176 | 1 | TTTAAAGTAGGAAAACAggc | cgg |
| 25327177 | 1 | TTAAAGTAGGAAAACAggcc | ggg |
| 25327184 | −1 | caggtgtgagccacggcgcc | cgg |
| 25327185 | 1 | GGAAAACAggccgggcgccg | tgg |
| 25327191 | −1 | gggattacaggtgtgagcca | cgg |
| 25327203 | −1 | tcccaaagtgttgggattac | agg |
| 25327211 | −1 | cctcagcctcccaaagtgtt | ggg |
| 25327212 | 1 | cacctgtaatcccaacactt | tgg |
| 25327212 | −1 | acctcagcctcccaaagtgt | tgg |
| 25327213 | 1 | acctgtaatcccaacacttt | ggg |
| 25327216 | 1 | tgtaatcccaacactttggg | agg |
| 25327222 | 1 | cccaacactttgggaggctg | agg |
| 25327225 | 1 | aacactttgggaggctgagg | tgg |
| 25327226 | 1 | acactttgggaggctgaggt | ggg |
| 25327240 | 1 | tgaggtgggcagatcacttg | agg |
| 25327245 | 1 | tgggcagatcacttgaggtc | agg |
| 25327263 | 1 | tcaggagttcaagaacagct | tgg |
| 25327272 | 1 | caagaacagcttggccaaca | tgg |
| 25327275 | −1 | gagacagggtttcaccatgt | tgg |
| 25327289 | −1 | ttgtgtttttagtagagaca | ggg |
| 25327290 | −1 | tttgtgtttttagtagagac | agg |
| 25327312 | 1 | taaaaacacaaacattagcc | agg |
| 25327317 | 1 | acacaaacattagccaggcg | tgg |
| 25327319 | −1 | ctggtgtgcaccaccacgcc | tgg |
| 25327320 | 1 | caaacattagccaggcgtgg | tgg |
| 25327338 | −1 | tcctgaatagctgggactac | tgg |
| 25327346 | −1 | cctcagcctcctgaatagct | ggg |
| 25327347 | −1 | gcctcagcctcctgaatagc | tgg |
| 25327348 | 1 | accagtagtcccagctattc | agg |
| 25327351 | 1 | agtagtcccagctattcagg | agg |
| 25327357 | 1 | cccagctattcaggaggctg | agg |
| 25327361 | 1 | gctattcaggaggctgaggc | agg |
| 25327368 | 1 | aggaggctgaggcaggaaaa | tgg |
| 25327378 | 1 | ggcaggaaaatggcttgaac | tgg |
| 25327379 | 1 | gcaggaaaatggcttgaact | ggg |
| 25327380 | 1 | caggaaaatggcttgaactg | ggg |
| 25327383 | 1 | gaaaatggcttgaactgggg | agg |
| 25327411 | −1 | ggagtgcagtggcacgatct | cgg |
| 25327422 | −1 | tcccccaggctggagtgcag | tgg |
| 25327429 | 1 | cgtgccactgcactccagcc | tgg |
| 25327430 | 1 | gtgccactgcactccagcct | ggg |
| 25327431 | 1 | tgccactgcactccagcctg | 335 |
| 25327432 | 1 | gccactgcactccagcctgg | ggg |
| 25327432 | −1 | tctccctctgtcccccaggc | tgg |
| 25327436 | −1 | ggagtctccctctgtccccc | agg |
| 25327439 | 1 | cactccagcctgggggacag | agg |
| 25327440 | 1 | actccagcctgggggacaga | ggg |
| 25327457 | −1 | tgttttgttttattttgaga | tgg |
| 25327483 | −1 | gctaatgtttttgtatgatt | tgg |
| 25327497 | 1 | aatcatacaaaaacattagc | tgg |
| 25327498 | 1 | atcatacaaaaacattagct | ggg |
| 25327503 | 1 | acaaaaacattagctgggtg | tgg |
| 25327506 | 1 | aaaacattagctgggtgtgg | tgg |
| 25327524 | −1 | tcccaagtagctgggattac | agg |
| 25327532 | −1 | cctcagcttcccaagtagct | ggg |
| 25327533 | 1 | tacctgtaatcccagctact | tgg |
| 25327533 | −1 | gcctcagcttcccaagtagc | tgg |
| 25327534 | 1 | acctgtaatcccagctactt | ggg |
| 25327543 | 1 | cccagctacttgggaagctg | agg |
| 25327564 | 1 | ggcagaattacttgaacccc | tgg |
| 25327565 | 1 | gcagaattacttgaacccct | ggg |
| 25327566 | 1 | cagaattacttgaacccctg | ggg |
| 25327567 | 1 | agaattacttgaacccctgg | ggg |
| 25327568 | 1 | gaattacttgaacccctggg | ggg |
| 25327569 | −1 | tcactgcaacctccccccag | ggg |
| 25327570 | −1 | ctcactgcaacctcccccca | ggg |
| 25327571 | 1 | ttacttgaacccctgggggg | agg |
| 25327571 | −1 | gctcactgcaacctcccccc | agg |
| 25327604 | −1 | ttgcccaggctggagtgtag | tgg |
| 25327611 | 1 | cttgccactacactccagcc | tgg |
| 25327612 | 1 | ttgccactacactccagcct | ggg |
| 25327614 | −1 | cctcactctgttgcccaggc | tgg |
| 25327618 | −1 | gtctcctcactctgttgccc | agg |
| 25327625 | 1 | ccagcctgggcaacagagtg | agg |
| 25327682 | 1 | aagaaaaaaaaaaGTAAACT | AGG |
| 25327709 | −1 | GGCTAGGGGAGTCTGTTGGC | AGG |
| 25327713 | −1 | CCGAGGCTAGGGGAGTCTGT | TGG |
| 25327723 | −1 | CTGGCCCTCACCGAGGCTAG | GGG |
| 25327724 | 1 | CCAACAGACTCCCCTAGCCT | CGG |
| 25327724 | −1 | ACTGGCCCTCACCGAGGCTA | GGG |
| 25327725 | −1 | CACTGGCCCTCACCGAGGCT | AGG |
| 25327729 | 1 | AGACTCCCCTAGCCTCGGTG | AGG |
| 25327730 | 1 | GACTCCCCTAGCCTCGGTGA | GGG |
| 25327730 | −1 | cagAACACTGGCCCTCACCG | AGG |
| 25327742 | 1 | CTCGGTGAGGGCCAGTGTTc | tgg |
| 25327742 | −1 | agatctgcctcccagAACAC | TGG |
| 25327743 | 1 | TCGGTGAGGGCCAGTGTTct | ggg |
| 25327746 | 1 | GTGAGGGCCAGTGTTctggg | agg |
| 25327775 | −1 | agcctgccagtgggtgaact | agg |
| 25327780 | 1 | tctagtcctagttcacccac | tgg |
| 25327784 | 1 | gtcctagttcacccactggc | agg |
| 25327784 | −1 | aagggcaccagcctgccagt | ggg |
| 25327785 | −1 | caagggcaccagcctgccag | tgg |
| 25327788 | 1 | tagttcacccactggcaggc | tgg |
| 25327797 | 1 | cactggcaggctggtgccct | tgg |
| 25327798 | 1 | actggcaggctggtgccctt | ggg |
| 25327802 | 1 | gcaggctggtgcccttgggc | agg |
| 25327802 | −1 | cagagaagcgacctgcccaa | ggg |
| 25327803 | −1 | ccagagaagcgacctgccca | agg |
| 25327814 | 1 | ccttgggcaggtcgcttctc | tgg |
| 25327815 | 1 | cttgggcaggtcgcttctct | ggg |
| 25327816 | 1 | ttgggcaggtcgcttctctg | ggg |
| 25327839 | −1 | GATTTGATctcattttatag | agg |
| 25327861 | −1 | agcacaaactcttAGAACAT | GGG |
| 25327862 | −1 | gagcacaaactcttAGAACA | TGG |
| 25327876 | 1 | TGTTCTaagagtttgtgctc | tgg |
| 25327892 | 1 | gctctggagtcagacagatc | tgg |
| 25327893 | 1 | ctctggagtcagacagatct | ggg |
| 25327910 | −1 | caagatcacagagctggcag | tgg |
| 25327916 | −1 | aagctacaagatcacagagc | tgg |
| 25327948 | 1 | ttcagtctcgtcatctgaca | tgg |
| 25327981 | 1 | aactgtctcactgtgttgtt | agg |
| 25327982 | 1 | actgtctcactgtgttgtta | ggg |
| 25327990 | 1 | actgtgttgttagggtttaa | agg |
| 25328042 | −1 | AACTCTGAAACCGGAAATCA | GGG |
| 25328043 | 1 | GTGTTAGCTACCCTGATTTC | CGG |
| 25328043 | −1 | GAACTCTGAAACCGGAAATC | AGG |
| 25328051 | −1 | GGACCACAGAACTCTGAAAC | CGG |
| 25328059 | 1 | TTTCCGGTTTCAGAGTTCTG | TGG |
| 25328072 | −1 | CACTGCATGTGGCATAAACT | GGG |
| 25328073 | −1 | TCACTGCATGTGGCATAAAC | TGG |
| 25328083 | −1 | CCATACAACGTCACTGCATG | TGG |
| 25328094 | 1 | CCACATGCAGTGACGTTGTA | TGG |
| 25328098 | 1 | ATGCAGTGACGTTGTATGGT | AGG |
| 25328104 | 1 | TGACGTTGTATGGTAGGCTG | TGG |
| 25328109 | 1 | TTGTATGGTAGGCTGTGGTG | TGG |
| 25328123 | −1 | gcatgcGCTGAGTTCTGAAG | TGG |
| 25328154 | 1 | tgcacagcttgcagaagaga | agg |
| 25328161 | 1 | cttgcagaagagaaggccag | agg |
| 25328166 | −1 | gagccttcttaggtctcctc | tgg |
| 25328174 | 1 | aggccagaggagacctaaga | agg |
| 25328176 | −1 | agtgttcgaagagccttctt | agg |
| 25328198 | 1 | tcttcgaacacttgaaagac | cgg |
| 25328206 | 1 | cacttgaaagaccggcatgt | agg |
| 25328206 | −1 | actgcgcccggcctacatgc | cgg |
| 25328210 | 1 | tgaaagaccggcatgtaggc | cgg |
| 25328211 | 1 | gaaagaccggcatgtaggcc | ggg |
| 25328218 | −1 | caggcgtgagtcactgcgcc | cgg |
| 25328237 | −1 | tccaaaactgctgggattac | agg |
| 25328245 | −1 | cctcgacctccaaaactgct | ggg |
| 25328246 | −1 | gcctcgacctccaaaactgc | tgg |
| 25328247 | 1 | gcctgtaatcccagcagttt | tgg |
| 25328250 | 1 | tgtaatcccagcagttttgg | agg |
| 25328256 | 1 | cccagcagttttggaggtcg | agg |
| 25328259 | 1 | agcagttttggaggtcgagg | cgg |
| 25328260 | 1 | gcagttttggaggtcgaggc | ggg |
| 25328263 | 1 | gttttggaggtcgaggcggg | tgs |
| 25328278 | 1 | gcgggtggatcacctgagtt | tgg |
| 25328279 | 1 | cgggtggatcacctgagttt | ggg |
| 25328279 | −1 | ggtatcaaactcccaaactc | agg |
| 25328300 | −1 | tttcaccttgttggtcaggc | tgg |
| 25328304 | −1 | ggggtttcaccttgttggtc | agg |
| 25328306 | 1 | tgataccagcctgaccaaca | agg |
| 25328309 | −1 | gagacggggtttcaccttgt | tgg |
| 25328323 | −1 | tgtattttttagtagagacg | ggg |
| 25328324 | −1 | ttgtattttttagtagagac | ggg |
| 25328325 | −1 | tttgtattttttagtagaga | cgg |
| 25328346 | 1 | taaaaaatacaaacattagc | tgg |
| 25328347 | 1 | aaaaaatacaaacattagct | ggg |
| 25328352 | 1 | atacaaacattagctgggca | tgg |
| 25328355 | 1 | caaacattagctgggcatgg | tgg |
| 25328358 | 1 | acattagctgggcatggtgg | cgg |
| 25328359 | 1 | cattagctgggcatggtggc | ggg |
| 25328373 | −1 | accggagtagctgggattac | agg |
| 25328381 | −1 | cctcaaccaccggagtagct | ggg |
| 25328382 | −1 | gcctcaaccaccggagtagc | tgg |
| 25328383 | 1 | gcctgtaatcccagctactc | cgg |
| 25328386 | 1 | tgtaatcccagctactccgg | tgg |
| 25328391 | −1 | agcaattctgcctcaaccac | cgg |
| 25328392 | 1 | cccagctactccggtggttg | agg |
| 25328411 | 1 | gaggcagaattgcttgaacc | cgg |
| 25328412 | 1 | aggcagaattgcttgaaccc | ggg |
| 25328415 | 1 | cagaattgcttgaacccggg | agg |
| 25328418 | −1 | cactgcaacctctgcctccc | ggg |
| 25328419 | −1 | tcactgcaacctctgcctcc | cgg |
| 25328421 | 1 | tgcttgaacccgggaggcag | agg |
| 25328464 | −1 | gtttcgctcttgtctcaggc | tgg |
| 25328468 | −1 | tggagtttcgctcttgtctc | agg |
| 25328488 | −1 | gttgtttgttttgtttgaga | tgg |
| 25328510 | −1 | tttttttggttttgtttggt | tgg |
| 25328514 | −1 | gttttttttttggttttgtt | tgg |
| 25328524 | −1 | ctacatgccagttttttttt | tgg |
| 25328528 | 1 | aacaaaaccaaaaaaaaaac | tgg |
| 25328575 | −1 | CTGGGCCTAGTTAAATTCTT | TGG |
| 25328581 | 1 | CTTCTCCAAAGAATTTAACT | AGG |
| 25328587 | 1 | CAAAGAATTTAACTAGGCCC | AGG |
| 25328588 | 1 | AAAGAATTTAACTAGGCCCA | GGG |
| 25328589 | 1 | AAGAATTTAACTAGGCCCAG | GGG |
| 25328592 | 1 | AATTTAACTAGGCCCAGGGG | AGG |
| 25328593 | −1 | atttATACTGCACCTCCCCT | GGG |
| 25328594 | −1 | aatttATACTGCACCTCCCC | TGG |
| 25328634 | 1 | aatctcaactgtctgccaaa | tgg |
| 25328638 | −1 | atgaagtagctcattccatt | tgg |
| 25328653 | 1 | atggaatgagctacttcata | tgg |
| 25328676 | −1 | ttgaatgcctccaaagacag | agg |
| 25328677 | 1 | agtagtgagtcctctgtctt | tgg |
| 25328680 | 1 | agtgagtcctctgtctttgg | agg |
| 25328702 | 1 | gcattcaaataaaagccaga | tgg |
| 25328706 | −1 | attgttgataaatggccatc | tgg |
| 25328714 | −1 | ttacatggattgttgataaa | tgg |
| 25328729 | −1 | atttcatctaacgttttaca | tgg |
| 25328756 | −1 | ggaagagatcttggatatat | agg |
| 25328765 | −1 | atctgaattggaagagatct | tgg |
| 25328777 | −1 | TTCTTtcataaaatctgaat | tgg |
| 25328797 | 1 | attttatgaAAGAATTTCTA | AGG |
| 25328819 | 1 | GTCTTTGTAATGAGACATTT | AGG |
| 25328849 | −1 | ATGAACCCACATACTGATTT | TGG |
| 25328854 | 1 | ATCAAGCCAAAATCAGTATG | TGG |
| 25328855 | 1 | TCAAGCCAAAATCAGTATGT | GGG |
| 25328905 | 1 | GCTTTTACAGTTTCCTCATT | TGG |
| 25328907 | −1 | TAAAATCCAACAGCCAAATG | AGG |
| 25328912 | 1 | CAGTTTCCTCATTTGGCTGT | TGG |
| 25328941 | −1 | TGAACAGGCCTTGTTTTTCT | TGG |
| 25328944 | 1 | AAAAGCATCCAAGAAAAACA | AGG |
| 25328956 | −1 | AAGTTGTCTTGTTTTTGAAC | AGG |
| 25328979 | −1 | CAAATGCAGGCAACAGTGAG | AGG |
| 25328992 | −1 | CGTTTCTCACGTACAAATGC | AGG |
| 25329033 | −1 | tccagtgcctgcgcGAACAT | TGG |
| 25329037 | 1 | AAAGTCTCCAATGTTCgcgc | agg |
| 25329043 | 1 | TCCAATGTTCgcgcaggcac | tgg |
| 25329058 | 1 | ggcactggagtcagagaaaa | tgg |
| 25329078 | −1 | CCTCAAAGagtggcagagaa | agg |
| 25329088 | −1 | GTGAGATTCTCCTCAAAGag | tgg |
| 25329089 | 1 | cctttctctgccactCTTTG | AGG |
| 25329110 | −1 | ATTCTACAGTGCATAATAAA | TGG |
| 25329150 | −1 | agatgttgttatgtggtaca | tgg |
| 25329157 | −1 | tttaccaagatgttgttatg | tgg |
| 25329164 | 1 | tgtaccacataacaacatct | tgg |
| 25329190 | 1 | acaacagactgcatatatga | tgg |
| 25329193 | 1 | acagactgcatatatgatgg | tgg |
| 25329209 | −1 | ATAAATTAACCTTAGCTTAC | TGG |
| 25329211 | 1 | ggtggtcATCCAGTAAGCTA | AGG |
| 25329285 | 1 | gtagtcttactctgtcaccc | agg |
| 25329291 | −1 | gtgccattgcactctagcct | ggg |
| 25329292 | −1 | ggtgccattgcactctagcc | tgg |
| 25329299 | 1 | tcacccaggctagagtgcaa | tgg |
| 25329310 | 1 | agagtgcaatggcaccatct | tgg |
| 25329313 | −1 | gaggttgcagtgagccaaga | tgg |
| 25329332 | −1 | tgcttgaacccaggaggtag | agg |
| 25329334 | 1 | tcactgcaacctctacctcc | tgg |
| 25329335 | 1 | cactgcaacctctacctcct | ggg |
| 25329338 | −1 | gagatttgcttgaacccagg | agg |
| 25329341 | −1 | caggagatttgcttgaaccc | agg |
| 25329360 | −1 | gctactttggaggctgaggc | agg |
| 25329364 | −1 | cccagctactttggaggctg | agg |
| 25329370 | −1 | tgtaatcccagctactttgg | agg |
| 25329373 | −1 | gcctgtaatcccagctactt | tgg |
| 25329374 | 1 | gcctcagcctccaaagtagc | tgg |
| 25329375 | 1 | cctcagcctccaaagtagct | ggg |
| 25329383 | 1 | tccaaagtagctgggattac | agg |
| 25329397 | −1 | aaaaattagccagatgtggt | ggg |
| 25329398 | −1 | aaaaaattagccagatgtgg | tgg |
| 25329399 | 1 | ttacaggcacccaccacatc | tgg |
| 25329401 | −1 | tacaaaaaattagccagatg | tgg |
| 25329428 | 1 | ttttgtatttttagtaaaga | tgg |
| 25329429 | 1 | tttgtatttttagtaaagat | ggg |
| 25329430 | 1 | ttgtatttttagtaaagatg | ggg |
| 25329444 | 1 | aagatggggtttcaccatgt | tgg |
| 25329447 | −1 | tgagatcagcctggccaaca | tgg |
| 25329449 | 1 | ggggtttcaccatgttggcc | agg |
| 25329456 | −1 | tcaggagtttgagatcagcc | tgg |
| 25329474 | −1 | cgggcagatcacttgaggtc | agg |
| 25329479 | −1 | cgagggggcagatcacttg | agg |
| 25329491 | 1 | ctcaagtgatctgcccgcct | cgg |
| 25329493 | −1 | gcactttgggaggccgaggc | ggg |
| 25329494 | −1 | agcactttgggaggccgagg | cgg |
| 25329497 | −1 | tccagcactttgggaggccg | agg |
| 25329503 | −1 | tgtggttccagcactttggg | agg |
| 25329506 | −1 | gcctgtggttccagcacttt | ggg |
| 25329507 | 1 | gcctcggcctcccaaagtgc | tgg |
| 25329507 | −1 | ggcctgtggttccagcactt | tgg |
| 25329516 | 1 | tcccaaagtgctggaaccac | agg |
| 25329521 | −1 | ggcacagtggctcaggcctg | tgg |
| 25329528 | −1 | AAggctgggcacagtggctc | agg |
| 25329534 | −1 | GCAAACAAggctgggcacag | tgg |
| 25329542 | −1 | TTAAAAAAGCAAACAAggct | ggg |
| 25329543 | −1 | GTTAAAAAAGCAAACAAggc | tgg |
| 25329547 | −1 | ATCTGTTAAAAAAGCAAACA | Agg |
| 25329642 | −1 | taaaaaTCTGAAGACTCTAG | TGG |
| 25329674 | 1 | tatactttttttttttgaaa | cgg |
| 25329694 | 1 | cggagtctcactctgtcacc | agg |
| 25329698 | 1 | gtctcactctgtcaccaggc | tgg |
| 25329701 | −1 | tgcggcactgcactccagcc | tgg |
| 25329719 | 1 | ggagtgcagtgccgcaatct | cgg |
| 25329719 | −1 | gttgcagtgagccgagattg | cgg |
| 25329741 | −1 | tgcttgaacctgggaggcgg | agg |
| 25329744 | 1 | cactgcaacctccgcctccc | agg |
| 25329744 | −1 | aattgcttgaacctgggagg | cgg |
| 25329747 | −1 | gagaattgcttgaacctggg | agg |
| 25329750 | −1 | caggagaattgcttgaacct | ggg |
| 25329751 | −1 | gcaggagaattgcttgaacc | tgg |
| 25329769 | −1 | gctactcgggaggctgaggc | agg |
| 25329773 | −1 | tccagctactcgggaggctg | agg |
| 25329779 | −1 | tgtaattccagctactcggg | agg |
| 25329782 | −1 | acttgtaattccagctactc | ggg |
| 25329783 | 1 | gcctcagcctcccgagtagc | tgg |
| 25329783 | −1 | cacttgtaattccagctact | cgg |
| 25329813 | −1 | atgcaaaaattagctgggtg | tgg |
| 25329818 | −1 | taaaaatgcaaaaattagct | ggg |
| 25329819 | −1 | gtaaaaatgcaaaaattagc | tgg |
| 25329838 | 1 | tttttgcatttttacttgac | agg |
| 25329839 | 1 | ttttgcatttttacttgaca | ggg |
| 25329853 | 1 | ttgacagggtttcaccatgt | tgg |
| 25329856 | −1 | tgaaactatcctagccaaca | tgg |
| 25329858 | 1 | agggtttcaccatgttggct | agg |
| 25329872 | 1 | ttggctaggatagtttcacc | agg |
| 25329879 | −1 | atcatgaggccaagagatcc | tgg |
| 25329881 | 1 | atagtttcaccaggatctct | tgg |
| 25329893 | −1 | gccgaggcaggctgatcatg | agg |
| 25329903 | 1 | gcctcatgatcagcctgcct | cgg |
| 25329905 | −1 | gcactttgggaggccgaggc | agg |
| 25329909 | −1 | cccagcactttgggaggccg | agg |
| 25329915 | −1 | tgtaatcccagcactttggg | agg |
| 25329918 | −1 | acctgtaatcccagcacttt | ggg |
| 25329919 | 1 | gcctcggcctcccaaagtgc | tgg |
| 25329919 | −1 | cacctgtaatcccagcactt | tgg |
| 25329920 | 1 | cctcggcctcccaaagtgct | ggg |
| 25329928 | 1 | tcccaaagtgctgggattac | agg |
| 25329946 | −1 | GAAGTATAggctgggcacgg | tgg |
| 25329949 | −1 | AGGGAAGTATAggctgggca | cgg |
| 25329954 | −1 | CAAAAAGGGAAGTATAggct | ggg |
| 25329955 | −1 | TCAAAAAGGGAAGTATAggc | tgg |
| 25329959 | −1 | GTATTCAAAAAGGGAAGTAT | Agg |
| 25329968 | −1 | CACCAAATGGTATTCAAAAA | GGG |
| 25329969 | −1 | ACACCAAATGGTATTCAAAA | AGG |
| 25329977 | 1 | TTCCCTTTTTGAATACCATT | TGG |
| 25329981 | −1 | TAATTCTTCAAAACACCAAA | TGG |
| 25330010 | 1 | AATTAACAGCTTTGTGAACG | TGG |
| 25330028 | 1 | CGTGGCAGTGCTTGTGATTC | AGG |
| 25330043 | −1 | GGTTCTCCCCTTGGTCTCAA | TGG |
| 25330046 | 1 | TCAGGCTTCCATTGAGACCA | AGG |
| 25330047 | 1 | CAGGCTTCCATTGAGACCAA | GGG |
| 25330048 | 1 | AGGCTTCCATTGAGACCAAG | GGG |
| 25330052 | −1 | CTGCAACCAGGTTCTCCCCT | TGG |
| 25330057 | 1 | TTGAGACCAAGGGGAGAACC | TGG |
| 25330064 | 1 | CAAGGGGAGAACCTGGTTGC | AGG |
| 25330064 | −1 | CGTCTGTTTGTCCTGCAACC | AGG |
| 25330076 | 1 | CTGGTTGCAGGACAAACAGA | CGG |
| 25330087 | 1 | ACAAACAGACGGACAGCGTG | TGG |
| 25330112 | 1 | GTGTTTAAATGCTCTTCTGA | AGG |
| 25330163 | −1 | GAAAACAATAATATAATCTT | GGG |
| 25330164 | −1 | AGAAAACAATAATATAATCT | TGG |
| 25330205 | 1 | TGTGTCACACTTTGCCAAAC | AGG |
| 25330208 | −1 | TTCATTTTCCACATCCTGTT | TGG |
| 25330211 | 1 | ACACTTTGCCAAACAGGATG | TGG |
| 25330226 | 1 | GGATGTGGAAAATGAATAAG | CGG |
| 25330236 | 1 | AATGAATAAGCGGTTTTCTT | AGG |
| 25330257 | 1 | GGCACTTCTTAACAGACAAT | TGG |
| 25330281 | −1 | TTTATGTGTTTCTTAAGCAA | TGG |
| 25330306 | −1 | AGCTATGTTCAGTGACTAAA | TGG |
| 25330327 | 1 | ACTGAACATAGCTATATGTA | TGG |
| 25330339 | 1 | TATATGTATGGTTGTTACTA | TGG |
| 25330340 | 1 | ATATGTATGGTTGTTACTAT | GGG |
| 25330365 | −1 | CCAGAATTTTCAAAGAAAAT | TGG |
| 25330376 | 1 | CCAATTTTCTTTGAAAATTC | TGG |
| 25330386 | 1 | TTGAAAATTCTGGCAGACCA | AGG |
| 25330392 | −1 | TATGTAAACAAAAAGAACCT | TGG |

In some embodiments, the gRNA target sequence is to exon 1 or exon 2 of the RHD gene. In some embodiments, the gRNA target sequence is a gRNA of Table 1 that induces a frameshift mutation to inactivate exon 1 or exon 2.

In some embodiments, expression of the RHD gene is partially or fully inactivated by an insertion or deletion within TCATGG, GAGGTG, AACTCG, AGTTTC, TTGGCT, or CACAGC of exon 2; CCGTGA of exon 3; GGGTAG or AGGGAA of exon 4; TTCGAT, TCAGCG, CATAGT, or ATCGAA of exon 5; CGTCGG or TCCGTC of exon 6; CGGCAA, CGGAGC, TACCGT, GCTTGC, or CTTGCT of exon 7; or GGTTCT or TCCTAC of exon 8 of the RHD gene.

Assays to test whether the RHD gene has been inactivated are known and described herein. In one embodiment, the resulting genetic modification of the RHD gene by PCR and the reduction of RhD antigen expression can be assays by FACS analysis. In another embodiment, RhD protein expression is detected using a Western blot of cells lysates probed with antibodies to the RhD protein. In another embodiment, reverse transcriptase polymerase chain reactions (RT-PCR) are used to confirm the presence of the inactivating genetic modification.

### G. CIITA

In some embodiments, the present technology disclosed herein modulates (e.g., reduces or eliminates) the expression of MHC II genes by targeting and modulating (e.g., reducing or eliminating) Class II transactivator (CIITA) expression. In some embodiments, the modulation occurs using a CRISPR/Cas system. CIITA is a member of the LR or nucleotide binding domain (NBD) leucine-rich repeat (LRR) family of proteins and regulates the transcription of MHC II by associating with the MHC enhanceosome.

In some embodiments, the target polynucleotide sequence of the present technology is a variant of CIITA. In some embodiments, the target polynucleotide sequence is a homolog of CIITA. In some embodiments, the target polynucleotide sequence is an ortholog of CIITA.

In some embodiments, reduced or eliminated expression of CIITA reduces or eliminates expression of one or more of the following MHC class II are HLA-DP, HLA-DM, HLA-DOA, HLA-DOB, HLA-DQ, and HLA-DR.

In some embodiments, the hypoimmunogenic T cells and non-activated T cells outlined herein comprise a genetic modification targeting the CIITA gene. In some embodiments, the genetic modification targeting the CIITA gene by a rare-cutting endonuclease comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOS:5184-36352 of Table 12 of WO2016183041, which is herein incorporated by reference. In some embodiments, the cell has a reduced ability to induce an immune response in a recipient subject.

In some embodiments, hypoimmunogenic T cells and non-activated T cells comprise a gene modification in the CIITA gene. In some embodiments, the gene modification affects one allele of the CIITA gene. In some embodiments, the gene modification affects two alleles of the CIITA gene. In some embodiments, the gene modification is an insertion, deletion, or disruption of the CIITA gene. In some embodiments, the gene modification is a homozygous modification of the CIITA gene. In some embodiments, the gene modification is a heterozygous modification of the CIITA gene.

Assays to test whether the CIITA gene has been inactivated are known and described herein. In one embodiment, the resulting genetic modification of the CIITA gene by PCR and the reduction of HLA-II expression can be assays by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating genetic modification.

### H. B2M

In certain embodiments, the present technology disclosed herein modulates (e.g., reduces or eliminates) the expression of MHC-I genes by targeting and modulating (e.g., reducing or eliminating) expression of the accessory chain B2M. In some embodiments, the modulation occurs using a CRISPR/Cas system. By modulating (e.g., reducing or deleting) expression of B2M, surface trafficking of MHC-I molecules is blocked, and the cell rendered hypoimmunogenic. In some embodiments, the cell has a reduced ability to induce an immune response in a recipient subject.

In some embodiments, the target polynucleotide sequence of the present technology is a variant of B2M. In some embodiments, the target polynucleotide sequence is a homolog of B2M. In some embodiments, the target polynucleotide sequence is an ortholog of B2M.

In some embodiments, decreased or eliminated expression of B2M reduces or eliminates expression of one or more of the following MHC I molecules—HLA-A, HLA-B, and HLA-C.

In some embodiments, the cells described herein comprise gene modifications at the gene locus encoding the B2M protein. In other words, the cells comprise a genetic modification at the B2M locus. In some instances, the nucleotide sequence encoding the B2M protein is set forth in RefSeq. No. NM_004048.4 and Genbank No. AB021288.1. In some instances, the B2M gene locus is described in NCBI Gene ID No. 567. In certain cases, the amino acid sequence of B2M is depicted as NCBI GenBank No. BAA35182.1. Additional descriptions of the B2M protein and gene locus can be found in Uniprot No. P61769, HGNC Ref. No. 914, and OMIM Ref. No. 109700.

In some embodiments, the hypoimmunogenic T cells and non-activated T cells outlined herein comprise a genetic modification targeting the B2M gene. In some embodiments, the genetic modification targeting the B2M gene by a rare-cutting endonuclease comprises a Cas protein or a polynucleotide encoding a Cas protein, and at least one guide ribonucleic acid sequence for specifically targeting the B2M gene. In some embodiments, the at least one guide ribonucleic acid sequence for specifically targeting the B2M gene is selected from the group consisting of SEQ ID NOS:81240-85644 of Table 15 of WO2016183041, which is herein incorporated by reference.

In some embodiments, hypoimmunogenic T cells and non-activated T cells comprise a gene modification in the B2M gene. In some embodiments, the gene modification affects one allele of the B2M gene. In some embodiments, the gene modification affects two alleles of the B2M gene. In some embodiments, the gene modification is an insertion, deletion, or disruption of the B2M gene. In some embodiments, the gene modification is a homozygous modification of the B2M gene. In some embodiments, the gene modification is a heterozygous modification of the B2M gene.

Assays to test whether the B2M gene has been inactivated are known and described herein. In one embodiment, the resulting genetic modification of the B2M gene by PCR and the reduction of HLA-I expression can be assays by FACS analysis. In another embodiment, B2M protein expression is detected using a Western blot of cells lysates probed with antibodies to the B2M protein. In another embodiment, reverse transcriptase polymerase chain reactions (RT-PCR) are used to confirm the presence of the inactivating genetic modification.

### I. Additional Tolerogenic Factors

In certain embodiments, one or more tolerogenic factors can be inserted or reinserted into genome-edited cells to create immune-privileged universal donor cells, such as universal donor stem cells, universal donor T cells, or universal donor cells. In certain embodiments, the hypoimmunogenic T cells and non-activated T cells disclosed herein have been further modified to express one or more tolerogenic factors. Exemplary tolerogenic factors include, without limitation, one or more of DUX4, CD200, HLA-G, HLA-E, HLA-C, HLA-E heavy chain, PD-L1, IDO1, CTLA4-Ig, IL-10, IL-35, FASL, Serpinb9, CCl21, and Mfge8. In some embodiments, the tolerogenic factors are selected from the group consisting of CD200, HLA-G, HLA-E, HLA-C, HLA-E heavy chain, PD-L1, IDO1, CTLA4-Ig, IL-10, IL-35, FASL, Serpinb9, CCl21, and Mfge8. In some embodiments, the tolerogenic factors are selected from the group consisting of DUX4, HLA-C, HLA-E, HLA-F, HLA-G, PD-L1, CTLA-4-Ig, C1-inhibitor, and IL-35. In some embodiments, the tolerogenic factors are selected from the group consisting of HLA-C, HLA-E, HLA-F, HLA-G, PD-L1, CTLA-4-Ig, C1-inhibitor, and IL-35.

In some instances, a gene editing system such as the CRISPR/Cas system is used to facilitate the insertion of tolerogenic factors, such as the tolerogenic factors into a safe harbor locus, such as the AAVS 1 locus, to actively inhibit immune rejection. In some instances, the tolerogenic factors are inserted into a safe harbor locus using an expression vector.

In some embodiments, the present disclosure provides a cell (e.g., a hypoimmunogenic T cell, a non-activated T cell, and derivatives thereof) or population thereof comprising a genome in which the cell genome has been modified to express CD47. In some embodiments, the present disclosure provides a method for altering a cell genome to express CD47. In some embodiments, at least one ribonucleic acid or at least one pair of ribonucleic acids may be utilized to facilitate the insertion of CD47 into a cell line. In some embodiments, the at least one ribonucleic acid or the at least one pair of ribonucleic acids is selected from the group consisting of SEQ ID NOS:200784-231885 of Table 29 of WO2016183041, which is herein incorporated by reference.

In some embodiments, the present disclosure provides a cell (e.g., a hypoimmunogenic T cell, a non-activated T cell, and derivatives thereof) or population thereof comprising a genome in which the cell genome has been modified to express HLA-C. In some embodiments, the present disclosure provides a method for altering a cell genome to express HLA-C. In some embodiments, at least one ribonucleic acid or at least one pair of ribonucleic acids may be utilized to facilitate the insertion of HLA-C into a cell line. In some embodiments, the at least one ribonucleic acid or the at least one pair of ribonucleic acids is selected from the group consisting of SEQ ID NOS:3278-5183 of Table 10 of WO2016183041, which is herein incorporated by reference.

In some embodiments, the present disclosure provides a cell (e.g., a hypoimmunogenic T cell, a non-activated T cell, and derivatives thereof) or population thereof comprising a genome in which the cell genome has been modified to express HLA-E. In some embodiments, the present disclosure provides a method for altering a cell genome to express HLA-E. In some embodiments, at least one ribonucleic acid or at least one pair of ribonucleic acids may be utilized to facilitate the insertion of HLA-E into a cell line. In some embodiments, the at least one ribonucleic acid or the at least one pair of ribonucleic acids is selected from the group consisting of SEQ ID NOS: 189859-193183 of Table 19 of WO2016183041, which is herein incorporated by reference.

In some embodiments, the present disclosure provides a cell (e.g., a hypoimmunogenic T cell, a non-activated T cell, and derivatives thereof) or population thereof comprising a genome in which the cell genome has been modified to express HLA-F. In some embodiments, the present disclosure provides a method for altering a cell genome to express HLA-F. In some embodiments, at least one ribonucleic acid or at least one pair of ribonucleic acids may be utilized to facilitate the insertion of HLA-F into a cell line. In some embodiments, the at least one ribonucleic acid or the at least one pair of ribonucleic acids is selected from the group consisting of SEQ ID NOS: 688808-399754 of Table 45 of WO2016183041, which is herein incorporated by reference.

In some embodiments, the present disclosure provides a cell (e.g., a hypoimmunogenic T cell, a non-activated T cell, and derivatives thereof) or population thereof comprising a genome in which the cell genome has been modified to express HLA-G. In some embodiments, the present disclosure provides a method for altering a cell genome to express HLA-G. In some embodiments, at least one ribonucleic acid or at least one pair of ribonucleic acids may be utilized to facilitate the insertion of HLA-G into a cell line, e.g., a stem cell line. In some embodiments, the at least one ribonucleic acid or the at least one pair of ribonucleic acids is selected from the group consisting of SEQ ID NOS: 188372-189858 of Table 18 of WO2016183041, which is herein incorporated by reference.

In some embodiments, the present disclosure provides a cell (e.g., a hypoimmunogenic T cell, a non-activated T cell, and derivatives thereof) or population thereof comprising a genome in which the cell genome has been modified to express PD-L1. In some embodiments, the present disclosure provides a method for altering a cell genome to express PD-L1. In some embodiments, at least one ribonucleic acid or at least one pair of ribonucleic acids may be utilized to facilitate the insertion of PD-L1 into a cell line, e.g., a stem cell line. In some embodiments, the at least one ribonucleic acid or the at least one pair of ribonucleic acids is selected from the group consisting of SEQ ID NOS: 193184-200783 of Table 21 of WO2016183041, which is herein incorporated by reference.

In some embodiments, the present disclosure provides a cell (e.g., a hypoimmunogenic T cell, a non-activated T cell, and derivatives thereof) or population thereof comprising a genome in which the cell genome has been modified to express CTLA4-Ig. In some embodiments, the present disclosure provides a method for altering a cell genome to express CTLA4-Ig. In some embodiments, at least one ribonucleic acid or at least one pair of ribonucleic acids may be utilized to facilitate the insertion of CTLA4-Ig into a cell line, e.g., a stem cell line. In some embodiments, the at least one ribonucleic acid or the at least one pair of ribonucleic acids is selected from any one disclosed in WO2016183041, including the sequence listing.

In some embodiments, the present disclosure provides a cell (e.g., a hypoimmunogenic T cell, a non-activated T cell, and derivatives thereof) or population thereof comprising a genome in which the cell genome has been modified to express CI-inhibitor. In some embodiments, the present disclosure provides a method for altering a cell genome to express CI-inhibitor. In some embodiments, at least one ribonucleic acid or at least one pair of ribonucleic acids may be utilized to facilitate the insertion of CI-inhibitor into a cell line, e.g., a stem cell line. In some embodiments, the at least one ribonucleic acid or the at least one pair of ribonucleic acids is selected from any one disclosed in WO2016183041, including the sequence listing.

In some embodiments, the present disclosure provides a cell (e.g., a hypoimmunogenic T cell, a non-activated T cell, and derivatives thereof) or population thereof comprising a genome in which the cell genome has been modified to express IL-35. In some embodiments, the present disclosure provides a method for altering a cell genome to express IL-35. In some embodiments, at least one ribonucleic acid or at least one pair of ribonucleic acids may be utilized to facilitate the insertion of IL-35 into a cell line, e.g., a stem cell line. In some embodiments, the at least one ribonucleic acid or the at least one pair of ribonucleic acids is selected from any one disclosed in WO2016183041, including the sequence listing.

In some embodiments, the tolerogenic factors are expressed in a cell using an expression vector. For example, the expression vector for expressing CD47 in a cell comprises a polynucleotide sequence encoding CD47. The expression vector can be an inducible expression vector. The expression vector can be a viral vector, such as but not limited to, a lentiviral vector.

In some embodiments, the present disclosure provides a cell (e.g., a hypoimmunogenic T cell, a non-activated T cell, and derivatives thereof) or population thereof comprising a genome in which the cell genome has been modified to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F. CD30, B7-H3, VISTA, TLT, PD-L2, CD58, CD2, HELIOS, and IDO1. In some embodiments, the present disclosure provides a method for altering a cell genome to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, and IDO1. In some embodiments, at least one ribonucleic acid or at least one pair of ribonucleic acids may be utilized to facilitate the insertion of the selected polypeptide into a cell line, e.g., a stem cell line. In some embodiments, the at least one ribonucleic acid or the at least one pair of ribonucleic acids is selected from any one disclosed in Appendices 1-47 and the sequence listing of WO2016183041, the disclosure is incorporated herein by references.

### J. Chimeric Antigen Receptors

Provided herein are hypoimmunogenic T cells and non-activated T cells, including hypoimmunogenic T cells and non-activated T cells differentiated from hypoimmune induced pluripotent stem cells and hypoimmunogenic T cells and non-activated T cells derived from primary T cells, comprising one or more chimeric antigen receptors (CARs). In some embodiments, a CAR is selected from the group consisting of a first generation CAR, a second generation CAR, a third generation CAR, and a fourth generation CAR.

In some embodiments, a hypoimmunogenic T cell described herein comprises one or more polynucleotides encoding one or more chimeric antigen receptors (CARs) comprising an antigen binding domain. In some embodiments, a hypoimmunogenic T cell described herein comprises one or more chimeric antigen receptors (CARs) comprising an antigen binding domain. In some embodiments, the polynucleotids are or comprise one or more chimeric antigen receptors (CARs) comprising an antigen binding domain. In some embodiments, the one or more CARs are or comprise a first generation CAR comprising an antigen binding domain, a transmembrane domain, and at least one signaling domain (e.g., one, two or three signaling domains). In some embodiments, the one or more CARs are or comprise a second generation CAR comprising an antigen binding domain, a transmembrane domain, and at least two signaling domains. In some embodiments, the one or more CARs are or comprise a third generation CAR comprising an antigen binding domain, a transmembrane domain, and at least three signaling domains. In some embodiments, the one or more CARs are or comprise a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an antibody, an antibody fragment, an scFv or a Fab.

In some instances, the cell expresses one or more nucleotide sequences encoding one or more CARs such that the nucleotide sequence is inserted into at least one allele of a safe harbor locus. In some instances, the cell expresses one or more nucleotide sequences encoding one or more CARs such that the nucleotide sequence(s) are inserted into at least one allele of an RHD locus. In some instances, the cell expresses one or more nucleotide sequences encoding one or more CARs such that the nucleotide sequence(s) are inserted into at least one allele of an AAVS1 locus. In some instances, the cell expresses one or more nucleotide sequences encoding one or more CARs such that the nucleotide sequence(s) are inserted into at least one allele of an CCR5 locus. In some instances, the cell expresses one or more nucleotide sequences encoding one or more CARs such that the nucleotide sequence(s) are inserted into at least one allele of a safe harbor gene locus, such as, but not limited to, a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, an LRP1 (CD91) gene locus, a HMGB1 gene locus, an ABO gene locus, an RHD gene locus, a FUT1 locus, and a KDM5D gene locus. In some instances, the cell expresses one or more nucleotide sequences encoding one or more CARs such that the nucleotide sequence(s) are inserted into at least one allele of a TRAC locus.

In some embodiments, the one or more nucleotide sequences encoding one or more CARs are delivered to a cell by a lentiviral vector. In some embodiments, the one or more nucleotide sequences encoding one or more CARs are introduced to an ex vivo cell. In some embodiments, the one or more nucleotide sequences encoding one or more CARs are introduced to an in vivo cell. In some embodiments, the one or more nucleotide sequences encoding one or more CARs are introduced into the cell's genome via a CRISPR/Cas-based system. In some embodiments, the one or more nucleotide sequences encoding one or more CARs are introduced into the cell's genome via a gene expression system that is not based on CRISPR/Cas technology.

### 1. Antigen Binding Domain (ABD) Targets an Antigen Characteristic of a Neoplastic or Cancer Cell

In some embodiments, the antigen binding domain (ABD) targets an antigen characteristic of a neoplastic cell. In other words, the antigen binding domain targets an antigen expressed by a neoplastic or cancer cell. In some embodiments, the ABD binds a tumor associated antigen. In some embodiments, the antigen characteristic of a neoplastic cell (e.g., antigen associated with a neoplastic or cancer cell) or a tumor associated antigen is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, Epidermal Growth Factor Receptors (EGFR) (including ErbB1/EGFR, ErbB2/HER2, ErbB3/HER3, and ErbB4/HER4), Fibroblast Growth Factor Receptors (FGFR) (including FGF1, FGF2. FGF3, FGF4, FGF5, FGF6, FGF7, FGF18, and FGF21) Vascular Endothelial Growth Factor Receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2, EphB3, EphB4, and EphB6), CXCR1, CXCR2, CXCR3, CXCR4, CXCR6, CCR1, CCR2, CCR3, CCR4, CCR5, CCR6, CCR8, CFTR, CIC-1, CIC-2, CIC-4, CIC-5, CIC-7, CIC-Ka, CIC-Kb, Bestrophins, TMEM16A, GABA receptor, glycin receptor, ABC transporters, NAV1.1, NAV1.2, NAV1.3, NAV1.4, NAV1.5, NAV1.6. NAV1.7, NAV1.8, NAV1.9, sphingosine-1-phosphate receptor (S1P1R), NMDA channel, transmembrane protein, multispan transmembrane protein, T-cell receptor motifs; T-cell alpha chains; T-cell β chains; T-cell γ chains; T-cell δ chains; CCR7; CD3; CD4; CD5; CD7; CD8; CD11b; CD11c; CD16; CD19; CD20; CD21; CD22; CD25; CD28; CD34; CD35; CD40; CD45RA; CD45RO; CD52; CD56; CD62L; CD68; CD80; CD95; CD117; CD127; CD133; CD137 (4-1 BB); CD163; F4/80; IL-4Ra; Sca-1; CTLA4; GITR; GARP; LAP; granzyme B; LFA-1; transferrin receptor; NKp46, perforin, CD4+; Th1; Th2; Th17; Th40; Th22; Th9; Tfh, Canonical Treg. FoxP3+; Tr1; Th3; Treg17; T_{RE}G; CDCP1, NT5E, EpCAM, CEA, gpA33, Mucins, TAG-72, Carbonic anhydrase IX, PSMA, Folate binding protein, Gangliosides (e.g., CD2, CD3, GM2), Lewis-γ², VEGF, VEGFR 1/2/3, αVβ3, α5β1, ErbB1/EGFR, ErbB1/HER2, ErB3, c-MET, IGF1R, EphA3, TRAIL-R1, TRAIL-R2, RANKL, FAP, Tenascin, PDL-1, BAFF, HDAC, ABL, FLT3, KIT, MET, RET, IL-1β, ALK, RANKL, mTOR, CTLA4, IL-6, IL-6R, JAK3, BRAF, PTCH, Smoothened, PIGF, ANPEP, TIMP1, PLAUR, PTPRJ, LTBR, or ANTXR1, Folate receptor alpha (FRa), ERBB2 (Her2/neu), EphA2, IL-13Ra2, epidermal growth factor receptor (EGFR), Mesothelin, TSHR, CD19, CD123, CD22, CD30, CD171, CS-1, CLL-1, CD33, EGFRvIII, GD2, GD3, BCMA, MUC16 (CA125), LICAM, LeY, MSLN, IL13Rα1, L1-CAM, Tn Ag, prostate specific membrane antigen (PSMA), ROR1, FLT3, FAP, TAG72, CD38, CD44v6, CEA, EPCAM, B7H3, KIT, interleukin-11 receptor a (IL-11Ra), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-1 receptor, CAIX, LMP2, gplOO, bcr-abl, tyrosinase, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLACl, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, ETV6-AML, sperm protein 17, XAGE1, Tie 2, MAD-CT-1, MAD-CT-2, Major histocompatibility complex class I-related gene protein (MR1), urokinase-type plasminogen activator receptor (uPAR), Fos-related antigen 1, p53, p53 mutant, prostein, survivin, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYPIB I, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, a neoantigen, CD133, CD15, CD184, CD24, CD56, CD26, CD29, CD44, HLA-A, HLA-B, HLA-C, (HLA-A,B,C) CD49f, CD151 CD340), CD200, tkrA, trkB, or trkC, or an antigenic fragment or antigenic portion thereof.

### 2. ABD Targets an Antigen Characteristic of a T Cell

In some embodiments, the antigen binding domain targets an antigen characteristic of a T cell. In some embodiments, the ABD binds an antigen associated with a T cell. In some instances, such an antigen is expressed by a T cell or is located on the surface of a T cell. In some embodiments, the antigen characteristic of a T cell or the T cell associated antigen is selected from a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, AKT1; AKT2; AKT3; ATF2; BCL10; CALM1; CD3D (CD3δ); CD3E (CD3ε); CD3G (CD3γ); CD4; CD8; CD28; CD45; CD80 (B7-1); CD86 (B7-2); CD247 (CD3ζ); CTLA4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP20K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10) (JNK3); MAPK11 (p38β); MAPK12 (p38γ); MAPK13 (p38δ); MAPK14 (p38α); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70).

### 3. ABD Targets an Antigen Characteristic of an Autoimmune or Inflammatory Disorder

In some embodiments, the antigen binding domain targets an antigen characteristic of an autoimmune or inflammatory disorder. In some embodiments, the ABD binds an antigen associated with an autoimmune or inflammatory disorder. In some instances, the antigen is expressed by a cell associated with an autoimmune or inflammatory disorder. In some embodiments, the autoimmune or inflammatory disorder is selected from chronic graft-vs-host disease (GVHD), lupus, arthritis, immune complex glomerulonephritis, goodpasture, uveitis, hepatitis, systemic sclerosis or scleroderma, type I diabetes, multiple sclerosis, cold agglutinin disease, Pemphigus vulgaris, Grave's disease, autoimmune hemolytic anemia, Hemophilia A, Primary Sjogren's Syndrome, thrombotic thrombocytopenia purrpura, neuromyelits optica, Evan's syndrome, IgM mediated neuropathy, cyroglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15(4):931-41) or xenosensitization from hematopoietic or solid organ transplantation, blood transfusions, pregnancy with fetal allosensitization, neonatal alloimmune thrombocytopenia, hemolytic disease of the new born, sensitization to foreign antigens such as can occur with replacement of inherited or acquired deficiency disorders treated with enzyme or protein replacement therapy, blood products, and gene therapy. In some embodiments, the antigen characteristic of an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, an antigen binding domain of a CAR binds to a ligand expressed on B cells, plasma cells, or plasmablasts. In some embodiments, an antigen binding domain of a CAR binds to CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5 or CD2. See US 2003/0077249; WO 2017/058753: WO 2017/058850, the contents of which are herein incorporated by reference.

### 4. ABD Targets an Antigen Characteristic of Senescent Cells

In some embodiments, the antigen binding domain targets an antigen characteristic of senescent cells, e.g., urokinase-type plasminogen activator receptor (uPAR). In some embodiments, the ABD binds an antigen associated with a senescent cell. In some instances, the antigen is expressed by a senescent cell. In some embodiments, the CAR may be used for treatment or prophylaxis of disorders characterized by the aberrant accumulation of senescent cells, e.g., liver and lung fibrosis, atherosclerosis, diabetes and osteoarthritis.

### 5. ABD Targets an Antigen Characteristic of an Infectious Disease

In some embodiments, the antigen binding domain targets an antigen characteristic of an infectious disease. In some embodiments, the ABD binds an antigen associated with an infectious disease. In some instances, the antigen is expressed by a cell affected by an infectious disease. In some embodiments, wherein the infectious disease is selected from HIV, hepatitis B virus, hepatitis C virus, Human herpes virus, Human herpes virus 8 (HHV-8, Kaposi sarcoma-associated herpes virus (KSHV)), Human T-lymphotrophic virus-1 (HTLV-1), Merkel cell polyomavirus (MCV), Simian virus 40 (SV40), Epstein-Barr virus, CMV, human papillomavirus. In some embodiments, the antigen characteristic of an infectious disease is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, HIV Env, gp120, or CD4-induced epitope on HIV-1 Env.

### 6. ABD Binds to a Cell Surface Antigen of a Cell

In some embodiments, an antigen binding domain binds to a cell surface antigen of a cell. In some embodiments, a cell surface antigen is characteristic of (e.g., expressed by) a particular or specific cell type. In some embodiments, a cell surface antigen is characteristic of more than one type of cell.

In some embodiments, a CAR antigen binding domain binds a cell surface antigen characteristic of a T cell, such as a cell surface antigen on a T cell. In some embodiments, an antigen characteristic of a T cell may be a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, an antigen binding domain of a CAR binds a T cell receptor. In some embodiments, a T cell receptor may be AKT1; AKT2; AKT3; ATF2; BCL10; CALM1; CD3D (CD3δ); CD3E (CD3ε); CD3G (CD3γ); CD4; CD8; CD28; CD45; CD80) (B7-1); CD86 (B7-2); CD247 (CD3ζ); CTLA4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10) (JNK3); MAPK11 (p38β); MAPK12 (p38γ); MAPK13 (p38δ); MAPK14 (p38α); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

### 7. Transmembrane Domain

In some embodiments, the CAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, the transmembrane domain comprises at least a transmembrane region(s) of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof, antigen binding domain binds

### 8. Signaling Domain or Plurality of Signaling Domains

In some embodiments, a CAR described herein comprises one or at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA4; Gi24/VISTA/B7-H5; ICOS/CD278; PD1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40) Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy 1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the at least one signaling domain comprises a CD3 zeta domain or an immunoreceptor tyrosine-based activation motif (ITAM), or functional variant thereof. In other embodiments, the at least one signaling domain comprises (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof: (ii) a CD28 domain or functional variant thereof; and (iii) a 4-1BB domain, or a CD134 domain, or functional variant thereof. In some embodiments, the at least one signaling domain comprises a (i) a CD3 zeta domain, or an immunoreceptor tyrosine-based activation motif (ITAM), or functional variant thereof: (ii) a CD28 domain or functional variant thereof: (iii) a 4-1BB domain, or a CD134 domain, or functional variant thereof; and (iv) a cytokine or costimulatory ligand transgene.

In some embodiments, the at least two signaling domains comprise a CD3 zeta domain or an immunoreceptor tyrosine-based activation motif (ITAM), or functional variant thereof. In other embodiments, the at least two signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof: (ii) a CD28 domain or functional variant thereof; and (iii) a 4-1BB domain, or a CD134 domain, or functional variant thereof. In some embodiments, the at least two signaling domains comprise a (i) a CD3 zeta domain, or an immunoreceptor tyrosine-based activation motif (ITAM), or functional variant thereof: (ii) a CD28 domain or functional variant thereof: (iii) a 4-1BB domain, or a CD134 domain, or functional variant thereof; and (iv) a cytokine or costimulatory ligand transgene.

In some embodiments, the at least three signaling domains comprise a CD3 zeta domain or an immunoreceptor tyrosine-based activation motif (ITAM), or functional variant thereof. In other embodiments, the at least three signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the least three signaling domains comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least three signaling domains comprise a (i) a CD3 zeta domain, or an immunoreceptor tyrosine-based activation motif (ITAM), or functional variant thereof: (ii) a CD28 domain or functional variant thereof: (iii) a 4-1BB domain, or a CD134 domain, or functional variant thereof; and (iv) a cytokine or costimulatory ligand transgene.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof.

In some embodiments, the CAR comprises a (i) a CD3 zeta domain, or an immunoreceptor tyrosine-based activation motif (ITAM), or functional variant thereof: (ii) a CD28 domain or functional variant thereof; and (iii) a 4-1BB domain, or a CD134 domain, or functional variant thereof.

In some embodiments, the CAR comprises (i) a CD3 zeta domain, or an immunoreceptor tyrosine-based activation motif (ITAM), or functional variant thereof: (ii) a CD28 domain, or a 4-1BB domain, or functional variant thereof, and/or (iii) a 4-1BB domain, or a CD134 domain, or functional variant thereof.

In some embodiments, the CAR comprises a (i) a CD3 zeta domain, or an immunoreceptor tyrosine-based activation motif (ITAM), or functional variant thereof: (ii) a CD28 domain or functional variant thereof: (iii) a 4-1BB domain, or a CD134 domain, or functional variant thereof; and (iv) a cytokine or costimulatory ligand transgene.

9. Domain which Upon Successful Signaling of the CAR Induces Expression of a Cytokine Gene

In some embodiments, a first, second, third, or fourth generation CAR further comprises a domain which upon successful signaling of the CAR induces expression of a cytokine gene. In some embodiments, a cytokine gene is endogenous or exogenous to a target cell comprising a CAR which comprises a domain which upon successful signaling of the CAR induces expression of a cytokine gene. In some embodiments, a cytokine gene encodes a pro-inflammatory cytokine. In some embodiments, a cytokine gene encodes IL-1, IL-2, IL-9, IL-12, IL-18, TNF, or IFN-gamma, or functional fragment thereof. In some embodiments, a domain which upon successful signaling of the CAR induces expression of a cytokine gene is or comprises a transcription factor or functional domain or fragment thereof. In some embodiments, a domain which upon successful signaling of the CAR induces expression of a cytokine gene is or comprises a transcription factor or functional domain or fragment thereof. In some embodiments, a transcription factor or functional domain or fragment thereof is or comprises a nuclear factor of activated T cells (NFAT), an NF-kB, or functional domain or fragment thereof. See, e.g., Zhang. C. et al., Engineering CAR T cells. Biomarker Research. 5:22 (2017); WO 2016126608; Sha, H. et al. Chimaeric antigen receptor T-cell therapy for tumour immunotherapy. Bioscience Reports Jan. 27, 2017, 37 (1).

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine and serine residues such as but not limited to glycine-serine doublets. In some embodiments, the CAR comprises two or more spacers, e.g., a spacer between the antigen binding domain and the transmembrane domain and a spacer between the transmembrane domain and a signaling domain.

In some embodiments, any one of the cells described herein comprises a nucleic acid encoding a CAR or a first generation CAR. In some embodiments, a first generation CAR comprises an antigen binding domain, a transmembrane domain, and signaling domain. In some embodiments, a signaling domain mediates downstream signaling during T cell activation.

In some embodiments, any one of the cells described herein comprises a nucleic acid encoding a CAR or a second generation CAR. In some embodiments, a second generation CAR comprises an antigen binding domain, a transmembrane domain, and two signaling domains. In some embodiments, a signaling domain mediates downstream signaling during T cell activation. In some embodiments, a signaling domain is a costimulatory domain. In some embodiments, a costimulatory domain enhances cytokine production, CAR T cell proliferation, and/or CAR T cell persistence during T cell activation.

In some embodiments, any one of the cells described herein comprises a nucleic acid encoding a CAR or a third generation CAR. In some embodiments, a third generation CAR comprises an antigen binding domain, a transmembrane domain, and at least three signaling domains. In some embodiments, a signaling domain mediates downstream signaling during T cell activation. In some embodiments, a signaling domain is a costimulatory domain. In some embodiments, a costimulatory domain enhances cytokine production, CAR T cell proliferation, and or CAR T cell persistence during T cell activation. In some embodiments, a third generation CAR comprises at least two costimulatory domains. In some embodiments, the at least two costimulatory domains are not the same.

In some embodiments, any one of the cells described herein comprises a nucleic acid encoding a CAR or a fourth generation CAR. In some embodiments, a fourth generation CAR comprises an antigen binding domain, a transmembrane domain, and at least two, three, or four signaling domains. In some embodiments, a signaling domain mediates downstream signaling during T cell activation. In some embodiments, a signaling domain is a costimulatory domain. In some embodiments, a costimulatory domain enhances cytokine production, CAR T cell proliferation, and or CAR T cell persistence during T cell activation.

### 10. ABD Comprising an Antibody or Antigen-Binding Portion Thereof

In some embodiments, a CAR antigen binding domain is or comprises an antibody or antigen-binding portion thereof. In some embodiments, a CAR antigen binding domain is or comprises an scFv or Fab. In some embodiments, a CAR antigen binding domain comprises an scFv or Fab fragment of a T-cell alpha chain antibody; T-cell β chain antibody; T-cell γ chain antibody; T-cell δ chain antibody; CCR7 antibody; CD3 antibody; CD4 antibody; CD5 antibody; CD7 antibody; CD8 antibody; CD11b antibody; CD11c antibody; CD16 antibody; CD19 antibody; CD20 antibody; CD21 antibody; CD22 antibody; CD25 antibody; CD28 antibody; CD34 antibody; CD35 antibody; CD40 antibody; CD45RA antibody; CD45RO antibody; CD52 antibody; CD56 antibody; CD62L antibody; CD68 antibody; CD80 antibody; CD95 antibody; CD117 antibody; CD127 antibody; CD133 antibody; CD137 (4-1 BB) antibody; CD163 antibody; F4/80 antibody; IL-4Ra antibody; Sca-1 antibody; CTLA4 antibody; GITR antibody GARP antibody; LAP antibody; granzyme B antibody; LFA-1 antibody; MR1 antibody; uPAR antibody; or transferrin receptor antibody.

In some embodiments, a CAR comprises a signaling domain which is a costimulatory domain. In some embodiments, a CAR comprises a second costimulatory domain. In some embodiments, a CAR comprises at least two costimulatory domains. In some embodiments, a CAR comprises at least three costimulatory domains. In some embodiments, a CAR comprises a costimulatory domain selected from one or more of CD27, CD28, 4-1BB, CD134/OX40, CD30, CD40, PD1, ICOS, lymphocyte function-associated antigen-1 (LFA-1), CD2, CD7, LIGHT, NKG2C, B7-H3, a ligand that specifically binds with CD83. In some embodiments, if a CAR comprises two or more costimulatory domains, two costimulatory domains are different. In some embodiments, if a CAR comprises two or more costimulatory domains, two costimulatory domains are the same.

In addition to the CARs described herein, various chimeric antigen receptors and nucleotide sequences encoding the same are known in the art and would be suitable for fusosomal delivery and reprogramming of target cells in vivo and in vitro as described herein. See, e.g., WO2013040557; WO2012079000; WO2016030414: Smith T, et al., Nature Nanotechnology. 2017. DOI: 10.1038/NNANO.2017.57, the disclosures of which are herein incorporated by reference.

### 11. Bispecific CARs

In certain embodiments, the at least one antigen binding domain is selected from the group consisting of an antibody, an antigen-binding portion thereof, an scFv, and a Fab. In some embodiments, the CAR is a bispecific CAR comprising two antigen binding domains that bind two different antigens. In some embodiments, the at least one antigen binding domain(s) binds to an antigen selected from the group consisting of CD19, CD22, and BCMA. In certain embodiments, the bispecific CAR binds to CD19 and CD22.

In some embodiments, the polynucleotide encoding the one or more CARs is carried by a lentiviral vector. In some embodiments, the one or more CARs are selected from the group consisting of a CD19-specific CAR, a CD20-specific CAR, a CD22-specific CAR, and combinations thereof. In some embodiments, the polynucleotide encoding the one or more CARs comprises a single bicistronic polynucleotide encoding both a CD19-specific CAR and a CD22-specific CAR. In some embodiments, the cells comprise a CD19-specific CAR encoded by one polynucleotide and a CD22-specific CAR encoded by another polynucleotide. In some embodiments, the CAR is a bispecific CAR. In some embodiments, the bispecific CAR is a CD19/CD20 bispecific CAR. In some embodiments, the bispecific CAR is a CD19/CD22 bispecific CAR. In some embodiments, the CAR is a bivalent CAR. In some embodiments, the bispecific CAR is a CD19/CD20 bivalent CAR. In some embodiments, the bispecific CAR is a CD19/CD22 bivalent CAR.

### 12. CAR

In certain embodiments, the cell may comprise an exogenous gene encoding a CAR. CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. The polycistronic vector of the present technology may be used to express one or more CARs in a host cell (e.g., a T cell) for use in cell-based therapies against various target antigens. The CARs expressed by the one or more expression cassettes may be the same or different. In these embodiments, the CAR may comprise an extracellular binding domain (also referred to as a “binder”) that specifically binds a target antigen, a transmembrane domain, and an intracellular signaling domain. In certain embodiments, the CAR may further comprise one or more additional elements, including one or more signal peptides, one or more extracellular hinge domains, and/or one or more intracellular costimulatory domains. Domains may be directly adjacent to one another, or there may be one or more amino acids linking the domains. The nucleotide sequence encoding a CAR may be derived from a mammalian sequence, for example, a mouse sequence, a primate sequence, a human sequence, or combinations thereof. In the cases where the nucleotide sequence encoding a CAR is non-human, the sequence of the CAR may be humanized. The nucleotide sequence encoding a CAR may also be codon-optimized for expression in a mammalian cell, for example, a human cell. In any of these embodiments, the nucleotide sequence encoding a CAR may be at least 80% identical (e.g., at least 80%, at least 85%, at least 90%, at least 95%, at least 96%, at least 97%, at least 98%, at least 99%, or 100% identical) to any of the nucleotide sequences disclosed herein. The sequence variations may be due to codon-optimalization, humanization, restriction enzyme-based cloning scars, and/or additional amino acid residues linking the functional domains, etc.

In certain embodiments, the CAR may comprise a signal peptide at the N-terminus. Non-limiting examples of signal peptides include CD8α signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 2 below.

TABLE 2
Exemplary sequences of signal peptides

| SEQ | | |
|---|---|---|
| ID NO: | Sequence | Description |
| 6 | MALPVTALLLPLALLLHAARP | CD8α signal |
| | | peptide |
| 7 | METDTLLLWVLLLWVPGSTG | IgK signal |
| | | peptide |
| 8 | MLLLVTSLLLCELPHPAFLLIP | GMCSFR-α (CSF2RA) |
| | | signal peptide |

In certain embodiments, the extracellular binding domain of the CAR may comprise one or more antibodies specific to one target antigen or multiple target antigens. The antibody may be an antibody fragment, for example, an scFv, or a single-domain antibody fragment, for example, a VHH. In certain embodiments, the scFv may comprise a heavy chain variable region (V_{H}) and a light chain variable region (V_{L}) of an antibody connected by a linker. The V_{H} and the V_{L} may be connected in either order, i.e., V_{H}-linker-V_{L} or V_{L}-linker-V_{H}. Non-limiting examples of linkers include Whitlow linker, (G₄S)ₙ (n can be a positive integer, e.g., 1, 2, 3, 4, 5, 6, etc.) linker, and variants thereof. In certain embodiments, the antigen may be an antigen that is exclusively or preferentially expressed on tumor cells, or an antigen that is characteristic of an autoimmune or inflammatory disease. Exemplary target antigens include, but are not limited to, CD5, CD19, CD20, CD22, CD23, CD30, CD70, Kappa, Lambda, and B cell maturation agent (BCMA), G-protein coupled receptor family C group 5 member D (GPRC5D) (associated with leukemias): CS1/SLAMF7, CD38, CD138, GPRC5D, TACI, and BCMA (associated with myelomas): GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, and ROR1 (associated with solid tumors). In any of these embodiments, the extracellular binding domain of the CAR can be codon-optimized for expression in a host cell or have variant sequences to increase functions of the extracellular binding domain.

In certain embodiments, the CAR may comprise a hinge domain, also referred to as a spacer. The terms “hinge” and “spacer” may be used interchangeably in the present disclosure. Non-limiting examples of hinge domains include CD8α hinge domain, CD28 hinge domain, IgG4 hinge domain, IgG4 hinge-CH2-CH3 domain, and variants thereof, the amino acid sequences of which are provided in Table 3 below.

TABLE 3
Exemplary sequences of hinge domains

| SEQ | | |
|---|---|---|
| ID NO: | Sequence | Description |
| 9 | TTTPAPRPPTPAPTIASQPLSLRPEACRPAA | CD8α hinge |
| | GGAVHTRGLDFACD | domain |
| 10 | IEVMYPPPYLDNEKSNGTIIHVKGKHLCPSP | CD28 hinge |
| | LFPGPSKP | domain |
| 113 | AAAIEVMYPPPYLDNEKSNGTIIHVKGKHL | CD28 hinge |
| | CPSPLFPGPSKP | domain |
| 11 | ESKYGPPCPPCP | IgG4 hinge |
| | | domain |
| 12 | ESKYGPPCPSCP | IgG4 hinge |
| | | domain |
| 13 | ESKYGPPCPPCPAPEFLGGPSVFLFPPKPKD | IgG4 hinge- |
| | TLMISRTPEVTCVVVDVSQEDPEVQFNWY | CH2—CH3 |
| | VDGVEVHNAKTKPREEQFNSTYRVVSVLT | domain |
| | VLHQDWLNGKEYKCKVSNKGLPSSIEKTIS | |
| | KAKGQPREPQVYTLPPSQEEMTKNQVSLT | |
| | CLVKGFYPSDIAVEWESNGQPENNYKTTPP | |
| | VLDSDGSFFLYSRLTVDKSRWQEGNVFSCS | |
| | VMHEALHNHYTQKSLSLSLGK | |

In certain embodiments, the transmembrane domain of the CAR may comprise a transmembrane region of the alpha, beta, or zeta chain of a T cell receptor, CD28, CD3ε, CD45, CD4, CD5, CD8, CD9, CD16, CD22, CD33, CD37, CD64, CD80, CD86, CD134, CD137, CD154, or a functional variant thereof, including the human versions of each of these sequences. In other embodiments, the transmembrane domain may comprise a transmembrane region of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or a functional variant thereof, including the human versions of each of these sequences. Table 4 provides the amino acid sequences of a few exemplary transmembrane domains.

TABLE 4
Exemplary sequences of transmembrane domains

| SEQ | | |
|---|---|---|
| ID NO: | Sequence | Description |
| 14 | IYIWAPLAGTCGVLLLSLVITL | CD8α transmembrane |
| | YC | domain |
| 15 | FWVLVVVGGVLACYSLLVTVAF | CD28 transmembrane |
| | IIFWV | domain |
| 114 | MFWVLVVVGGVLACYSLLVTVA | CD28 transmembrane |
| | FIIFWV | domain |

In certain embodiments, the intracellular signaling domain and/or intracellular costimulatory domain of the CAR may comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/K1M-1/HAVCR, TIM-4, TSLP, TSLP R, lymphocyte function associated antigen-1 (LFA-1), NKG2C, CD3ζ, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain and/or intracellular costimulatory domain comprises one or more signaling domains selected from a CD3ζ domain, an ITAM, a CD28 domain, 4-1BB domain, or a functional variant thereof. Table 5 provides the amino acid sequences of a few exemplary intracellular costimulatory and/or signaling domains. In certain embodiments, as in the case of tisagenlecleucel as described below, the CD3ζ signaling domain of SEQ ID NO:18 may have a mutation, e.g., a glutamine (Q) to lysine (K) mutation, at amino acid position 14 (see SEQ ID NO:115).

TABLE 5
Exemplary sequences of intracellular costimulatory and/or signaling domains

| SEQ ID NO: | Sequence | Description |
|---|---|---|
| 16 | KRGRKKLLYIFKQPFMRPVQTTQEEDG | 4-1BB costimulatory domain |
| | CSCRFPEEEEGGCEL | |
| 17 | RSKRSRLLHSDYMNMTPRRPGPTRKHY | CD28 costimulatory domain |
| | QPY APPRDFAAYRS | |
| 18 | RVKFSRSADAPAYQQGQNQLYNELNL | CD3ζ signaling domain |
| | GRREEYDVLDKRRGRDPEMGGKPRRK | |
| | NPQEGLYNELQKDKMAEAYSEIGMKG | |
| | ERRRGKGHDGLYQGLSTATKDTYDAL | |
| | HMQALPPR | |
| 115 | RVKFSRSADAPAYKQGQNQLYNELNL | CD32 signaling domain (with |
| | GRREEYDVLDKRRGRDPEMGGKPRRK | Q to K mutation at position 14) |
| | NPQEGLYNELQKDKMAEAYSEIGMKG | |
| | ERRRGKGHDGLYQGLSTATKDTYDAL | |
| | HMQALPPR | |

In certain embodiments where the polycistronic vector encodes two or more CARs, the two or more CARs may comprise the same functional domains, or one or more different functional domains, as described. For example, the two or more CARs may comprise different signal peptides, extracellular binding domains, hinge domains, transmembrane domains, costimulatory domains, and/or intracellular signaling domains, in order to minimize the risk of recombination due to sequence similarities. Or, alternatively, the two or more CARs may comprise the same domains. In the cases where the same domain(s) and/or backbone are used, it is optional to introduce codon divergence at the nucleotide sequence level to minimize the risk of recombination.

### CD19 CAR

In some embodiments, the CAR is a CD19 CAR, and in these embodiments, the polycistronic vector comprises an expression cassette that contains a nucleotide sequence encoding a CD19 CAR. In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the signal peptide of the CD19 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34(16-17): 1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 6 below. In some embodiments, the CD19-specific scFv comprises or consists of an amino acid sequence set forth in SEQ ID NO: 19, 20, or 25, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:19, 20, or 25. In some embodiments, the CD19-specific scFv may comprise one or more CDRs having amino acid sequences set forth in SEQ ID NOs: 21-23 and 26-28. In some embodiments, the CD19-specific scFv may comprise a light chain with one or more CDRs having amino acid sequences set forth in SEQ ID NOs: 21-23. In some embodiments, the CD19-specific scFv may comprise a heavy chain with one or more CDRs having amino acid sequences set forth in SEQ ID NOs: 26-28. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the V_{H} and the V_{L} portions of the scFv is a Whitlow linker having an amino acid sequence set forth in SEQ ID NO:24. In some embodiments, the Whitlow linker may be replaced by a different linker, for example, a 3×G₄S linker having an amino acid sequence set forth in SEQ ID NO:30, which gives rise to a different FMC63-derived scFv having an amino acid sequence set forth in SEQ ID NO:29. In certain of these embodiments, the CD19-specific scFv comprises or consists of an amino acid sequence set forth in SEQ ID NO:29 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:29.

TABLE 6
Exemplary sequences of anti-CD19 scFv and components

| SEQ ID NO: | Amino Acid Sequence | Description |
|---|---|---|
| 19 | DIQMTQTTSSLSASLGDRVTISCRAS | Anti-CD19 FMC63 scFv |
| | QDISKYLNWYQQKPDGTVKLLIYHT | entire sequence, with |
| | SRLHSGVPSRFSGSGSGTDYSLTISN | Whitlow linker |
| | LEQEDIATYFCQQGNTLPYTFGGGT | |
| | KLEITGSTSGSGKPGSGEGSTKGEVK | |
| | LQESGPGLVAPSQSLSVTCTVSGVSL | |
| | PDYGVSWIRQPPRKGLEWLGVIWGS | |
| | ETTYYNSALKSRLTIIKDNSKSQVFL | |
| | KMNSLQTDDTAIYYCAKHYYYGGS | |
| | YAMDYWGQGTSVTVSS | |
| 20 | DIQMTQTTSSLSASLGDRVTISCRAS | Anti-CD19 FMC63 scFv |
| | QDISKYLNWYQQKPDGTVKLLIYHT | light chain variable region |
| | SRLHSGVPSRFSGSGSGTDYSLTISN | |
| | LEQEDIATYFCQQGNTLPYTFGGGT | |
| | KLEIT | |
| 21 | QDISKY | Anti-CD19 FMC63 scFv |
| | | light chain CDR1 |
| 22 | HTS | Anti-CD19 FMC63 scFv |
| | | light chain CDR2 |
| 23 | QQGNTLPYT | Anti-CD19 FMC63 scFv |
| | | light chain CDR3 |
| 24 | GSTSGSGKPGSGEGSTKG | Whitlow linker |
| 25 | EVKLQESGPGLVAPSQSLSVTCTVS | Anti-CD19 FMC63 scFv |
| | GVSLPDYGVSWIRQPPRKGLEWLG | heavy chain variable |
| | VIWGSETTYYNSALKSRLTIIKDNSK | region |
| | SQVFLKMNSLQTDDTAIYYCAKHY | |
| | YYGGSYAMDYWGQGTSVTVSS | |
| 26 | GVSLPDYG | Anti-CD19 FMC63 scFv |
| | | heavy chain CDR1 |
| 27 | IWGSETT | Anti-CD19 FMC63 scFv |
| | | heavy chain CDR2 |
| 28 | AKHYYYGGSYAMDY | Anti-CD19 FMC63 scFv |
| | | heavy chain CDR3 |
| 29 | DIQMTQTTSSLSASLGDRVTISCRAS | Anti-CD19 FMC63 scFv |
| | QDISKYLNWYQQKPDGTVKLLIYHT | entire sequence, with |
| | SRLHSGVPSRFSGSGSGTDYSLTISN | 3xG4S linker |
| | LEQEDIATYFCQQGNTLPYTFGGGT | |
| | KLEITGGGGSGGGGSGGGGSEVKLQ | |
| | ESGPGLVAPSQSLSVTCTVSGVSLPD | |
| | YGVSWIRQPPRKGLEWLGVIWGSET | |
| | TYYNSALKSRLTIIKDNSKSQVFLK | |
| | MNSLQTDDTAIYYCAKHYYYGGSY | |
| | AMDYWGQGTSVTVSS | |
| 30 | GGGGSGGGGSGGGGS | 3xG4S linker |

In some embodiments, the extracellular binding domain of the CD19 CAR is derived from an antibody specific to CD19, including, for example, SJ25C1 (Bejcek et al., Cancer Res. 55:2346-2351 (1995)), HD37 (Pezutto et al., J. Immunol. 138(9):2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148(10): 2983-2987 (1992)), and CLB-CD19 (De Rie Cell. Immunol. 118:368-381(1989)). In any of these embodiments, the extracellular binding domain of the CD19 CAR can comprise or consist of the V_{H}, the V_{L}, and/or one or more CDRs of any of the antibodies.

In some embodiments, the hinge domain of the CD19 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO: 10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO: 11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO: 13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the CD19 CAR comprises a CD8α transmembrane domain, for example, a human CD8α transmembrane domain. In some embodiments, the CD8α transmembrane domain comprises or consists of an amino acid sequence set forth in SEQ ID NO: 14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 15.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17. In some embodiments, the intracellular costimulatory domain of the CD19 CAR comprises a 4-1BB costimulatory domain and a CD28 costimulatory domain as described.

In some embodiments, the intracellular signaling domain of the CD19 CAR comprises a CD3 zeta (ζ) signaling domain. CD3ζ associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In some embodiments, the CD3ζ signaling domain is human. In some embodiments, the CD3ζ signaling domain comprises or consists of an amino acid sequence set forth in SEQ ID NO: 18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 18.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:19 or SEQ ID NO:29, the CD8α hinge domain of SEQ ID NO:9, the CD8α transmembrane domain of SEQ ID NO: 14, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:19 or SEQ ID NO:29, the IgG4 hinge domain of SEQ ID NO: 11 or SEQ ID NO: 12, the CD28 transmembrane domain of SEQ ID NO: 15, the 4-1BB costimulatory domain of SEQ ID NO: 16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:19 or SEQ ID NO:29, the CD28 hinge domain of SEQ ID NO: 10, the CD28 transmembrane domain of SEQ ID NO: 15, the CD28 costimulatory domain of SEQ ID NO: 17, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR as set forth in SEQ ID NO:116 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:116 (see Table 7). The encoded CD19 CAR has a corresponding amino acid sequence set forth in SEQ ID NO: 117 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:117, with the following components; CD8α signal peptide, FMC63 scFv (V_{L}-Whitlow linker-V_{H}), CD8α hinge domain, CD8α transmembrane domain, 4-1 BB costimulatory domain, and CD3ζ signaling domain.

In some embodiments, the polycistronic vector comprises an expression cassette that contains a nucleotide sequence encoding a commercially available embodiment of CD19 CAR. Non-limiting examples of commercially available embodiments of CD19 CARs expressed and/or encoded by T cells include tisagenlecleucel, lisocabtagene maraleucel, axicabtagene ciloleucel, and brexucabtagene autoleucel.

In some embodiments, the polycistronic vector comprises an expression cassette that contains a nucleotide sequence encoding tisagenlecleucel or portions thereof. Tisagenlecleucel comprises a CD19 CAR with the following components; CD8α signal peptide, FMC63 scFv (V_{L}-3×G₄S linker-V_{H}), CD8α hinge domain, CD8α transmembrane domain, 4-1BB costimulatory domain, and CD3ζ signaling domain. The nucleotide and amino acid sequence of the CD19 CAR in tisagenlecleucel are provided in Table 7, with annotations of the sequences provided in Table 8.

In some embodiments, the polycistronic vector comprises an expression cassette that contains a nucleotide sequence encoding lisocabtagene maraleucel or portions thereof. Lisocabtagene maraleucel comprises a CD19 CAR with the following components: GMCSFR-α or CSF2RA signal peptide, FMC63 scFv (V_{L}-Whitlow linker-V_{H}), IgG4 hinge domain, CD28 transmembrane domain, 4-1BB costimulatory domain, and CD3ζ signaling domain. The nucleotide and amino acid sequence of the CD19 CAR in lisocabtagene maraleucel are provided in Table 7, with annotations of the sequences provided in Table 9.

In some embodiments, the polycistronic vector comprises an expression cassette that contains a nucleotide sequence encoding axicabtagene ciloleucel or portions thereof. Axicabtagene ciloleucel comprises a CD19 CAR with the following components: GMCSFR-α or CSF2RA signal peptide, FMC63 scFv (V_{L}-Whitlow linker-V_{H}), CD28 hinge domain, CD28 transmembrane domain, CD28 costimulatory domain, and CD3ζ signaling domain. The nucleotide and amino acid sequence of the CD19 CAR in axicabtagene ciloleucel are provided in Table 7, with annotations of the sequences provided in Table 10.

In some embodiments, the polycistronic vector comprises an expression cassette that contains a nucleotide sequence encoding brexucabtagene autoleucel or portions thereof. Brexucabtagene autoleucel comprises a CD19 CAR with the following components: GMCSFR-α signal peptide, FMC63 scFv, CD28 hinge domain, CD28 transmembrane domain, CD28 costimulatory domain, and CD3ζ signaling domain.

In some embodiments, the polycistronic vector comprises an expression cassette that contains a nucleotide sequence encoding a CD19 CAR as set forth in SEQ ID NO: 31, 33, or 35, or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 31, 33, or 35. The encoded CD19 CAR has a corresponding amino acid sequence set forth in SEQ ID NO: 32, 34, or 36, respectively, or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 32, 34, or 36, respectively.

TABLE 7
Exemplary sequences of CD19 CARs

| SEQ ID NO: | Sequence | Description |
|---|---|---|
| 116 | atggccttaccagtgaccgccttgctcctgccgctggccttgctgct | Exemplary CD19 |
| | ccacgccgccaggccggacatccagatgacacagactacatcctc | CAR nucleotide |
| | cctgtctgcctctctgggagacagagtcaccatcagttgcagggca | sequence |
| | agtcaggacattagtaaatatttaaattggtatcagcagaaaccagat | |
| | ggaactgttaaactcctgatctaccatacatcaagattacactcagg | |
| | agtcccatcaaggttcagtggcagtgggtctggaacagattattctc | |
| | tcaccattagcaacctggagcaagaagatattgccacttacttttgcc | |
| | aacagggtaatacgcttccgtacacgttcggaggggggaccaagc | |
| | tggagatcacaggctccacctctggatccggcaagcccggatctg | |
| | gcgagggatccaccaagggcgaggtgaaactgcaggagtcagg | |
| | acctggcctggtggcgccctcacagagcctgtccgtcacatgcact | |
| | gtctcaggggtctcattacccgactatggtgtaagctggattcgcca | |
| | gcctccacgaaagggtctggagtggctgggagtaatatggggtag | |
| | tgaaaccacatactataattcagctctcaaatccagactgaccatcat | |
| | caaggacaactccaagagccaagttttcttaaaaatgaacagtctgc | |
| | aaactgatgacacagccatttactactgtgccaaacattattactacg | |
| | gtggtagctatgctatggactactggggccaaggaacctcagtcac | |
| | cgtctcctcaaccacgacgccagcgccgcgaccaccaacaccgg | |
| | cgcccaccatcgcgtcgcagcccctgtccctgcgcccagaggcgt | |
| | gccggccagcggcggggggcgcagtgcacacgagggggctgg | |
| | acttcgcctgtgatatctacatctgggcgcccttggccgggacttgt | |
| | ggggtccttctcctgtcactggttatcaccctttactgcaaacggggc | |
| | agaaagaaactcctgtatatattcaaacaaccatttatgagaccagta | |
| | caaactactcaagaggaagatggctgtagctgccgatttccagaag | |
| | aagaagaaggaggatgtgaactgagagtgaagttcagcaggagc | |
| | gcagacgcccccgcgtaccagcagggccagaaccagctctataa | |
| | cgagctcaatctaggacgaagagaggagtacgatgttttggacaa | |
| | gagacgtggccgggaccctgagatggggggaaagccgagaag | |
| | gaagaaccctcaggaaggcctgtacaatgaactgcagaaagataa | |
| | gatggcggaggcctacagtgagattgggatgaaaggcgagcgcc | |
| | ggaggggcaaggggcacgatggcctttaccagggtctcagtaca | |
| | gccaccaaggacacctacgacgcccttcacatgcaggccctgccc | |
| | cctcgc | |
| 117 | MALPVTALLLPLALLLHAARPDIQMTQTTS | Exemplary CD19 |
| | SLSASLGDRVTISCRASQDISKYLNWYQQK | CAR amino acid |
| | PDGTVKLLIYHTSRLHSGVPSRFSGSGSGT | sequence |
| | DYSLTISNLEQEDIATYFCQQGNTLPYTFG | |
| | GGTKLEITGSTSGSGKPGSGEGSTKGEVKL | |
| | QESGPGLVAPSQSLSVTCTVSGVSLPDYGV | |
| | SWIRQPPRKGLEWLGVIWGSETTYYNSAL | |
| | KSRLTIIKDNSKSQVFLKMNSLQTDDTAIY | |
| | YCAKHYYYGGSYAMDYWGQGTSVTVSST | |
| | TTPAPRPPTPAPTIASQPLSLRPEACRPAAG | |
| | GAVHTRGLDFACDIYIWAPLAGTCGVLLLS | |
| | LVITLYCKRGRKKLLYIFKQPFMRPVQTTQ | |
| | EEDGCSCRFPEEEEGGCELRVKFSRSADAP | |
| | AYQQGQNQLYNELNLGRREEYDVLDKRR | |
| | GRDPEMGGKPRRKNPQEGLYNELQKDKM | |
| | AEAYSEIGMKGERRRGKGHDGLYQGLSTA | |
| | TKDTYDALHMQALPPR | |
| 31 | atggccttaccagtgaccgccttgctcctgccgctggccttgctgct | Tisagenlecleucel |
| | ccacgccgccaggccggacatccagatgacacagactacatcctc | CD19 CAR |
| | cctgtctgcctctctgggagacagagtcaccatcagttgcagggca | nucleotide |
| | agtcaggacattagtaaatatttaaattggtatcagcagaaaccagat | sequence |
| | ggaactgttaaactcctgatctaccatacatcaagattacactcagg | |
| | agtcccatcaaggttcagtggcagtgggtctggaacagattattctc | |
| | tcaccattagcaacctggagcaagaagatattgccacttacttttgcc | |
| | aacagggtaatacgcttccgtacacgttcggaggggggaccaagc | |
| | tggagatcacaggtggcggtggctcgggcggtggtgggtcgggt | |
| | ggcggcggatctgaggtgaaactgcaggagtcaggacctggcct | |
| | ggtggcgccctcacagagcctgtccgtcacatgcactgtctcagg | |
| | ggtctcattacccgactatggtgtaagctggattcgccagcctccac | |
| | gaaagggtctggagtggctgggagtaatatggggtagtgaaacca | |
| | catactataattcagctctcaaatccagactgaccatcatcaaggac | |
| | aactccaagagccaagttttcttaaaaatgaacagtctgcaaactga | |
| | tgacacagccatttactactgtgccaaacattattactacggtggtag | |
| | ctatgctatggactactggggccaaggaacctcagtcaccgtctcct | |
| | caaccacgacgccagcgccgcgaccaccaacaceggegcccac | |
| | catcgcgtcgcagcccctgtccctgcgcccagaggcgtgccggc | |
| | cagcggcggggggcgcagtgcacacgagggggctggacttcgc | |
| | ctgtgatatctacatctgggcgcccttggccgggacttgtggggtcc | |
| | ttctcctgtcactggttatcaccctttactgcaaacggggcagaaag | |
| | aaactcctgtatatattcaaacaaccatttatgagaccagtacaaact | |
| | actcaagaggaagatggctgtagctgccgatttccagaagaagaa | |
| | gaaggaggatgtgaactgagagtgaagttcagcaggagcgcaga | |
| | cgcccccgcgtacaagcagggccagaaccagctctataacgagc | |
| | tcaatctaggacgaagagaggagtacgatgttttggacaagagac | |
| | gtggccgggaccctgagatggggggaaagccgagaaggaaga | |
| | accctcaggaaggcctgtacaatgaactgcagaaagataagatgg | |
| | cggaggcctacagtgagattgggatgaaaggcgagcgccggag | |
| | gggcaaggggcacgatggcctttaccagggtctcagtacagccac | |
| | caaggacacctacgacgcccttcacatgcaggccctgccccctcg | |
| | c | |
| 32 | MALPVTALLLPLALLLHAARPDIQMTQTTS | Tisagenlecleucel |
| | SLSASLGDRVTISCRASQDISKYLNWYQQK | CD19 CAR amino |
| | PDGTVKLLIYHTSRLHSGVPSRFSGSGSGT | acid sequence |
| | DYSLTISNLEQEDIATYFCQQGNTLPYTFG | |
| | GGTKLEITGGGGSGGGGSGGGGSEVKLQE | |
| | SGPGLVAPSQSLSVTCTVSGVSLPDYGVSW | |
| | IRQPPRKGLEWLGVIWGSETTYYNSALKSR | |
| | LTIIKDNSKSQVFLKMNSLQTDDTAIYYCA | |
| | KHYYYGGSYAMDYWGQGTSVTVSSTTTP | |
| | APRPPTPAPTIASQPLSLRPEACRPAAGGAV | |
| | HTRGLDFACDIYIWAPLAGTCGVLLLSLVI | |
| | TLYCKRGRKKLLYIFKQPFMRPVQTTQEED | |
| | GCSCRFPEEEEGGCELRVKFSRSADAPAYK | |
| | QGQNQLYNELNLGRREEYDVLDKRRGRD | |
| | PEMGGKPRRKNPQEGLYNELQKDKMAEA | |
| | YSEIGMKGERRRGKGHDGLYQGLSTATKD | |
| | TYDALHMQALPPR | |
| 33 | atgctgctgctggtgaccagcctgctgctgtgcgagctgccccacc | Lisocabtagene |
| | ccgcctttctgctgatccccgacatccagatgacccagaccacctc | maraleucel CD19 |
| | cagcctgagcgccagcctgggcgaccgggtgaccatcagctgcc | CAR nucleotide |
| | gggccagccaggacatcagcaagtacctgaactggtatcagcag | sequence |
| | aagcccgacggcaccgtcaagctgctgatctaccacaccagccg | |
| | gctgcacagcggcgtgcccagccggtttagcggcagcggctccg | |
| | gcaccgactacagcctgaccatctccaacctggaacaggaagata | |
| | tcgccacctacttttgccagcagggcaacacactgccctacaccttt | |
| | ggcggcggaacaaagctggaaatcaccggcagcacctccggca | |
| | gcggcaagcctggcagcggcgagggcagcaccaagggcgagg | |
| | tgaagctgcaggaaagcggccctggcctggtggcccccagccag | |
| | agcctgagcgtgacctgcaccgtgagcggcgtgagcctgcccga | |
| | ctacggcgtgagctggatccggcagccccccaggaagggcctgg | |
| | aatggctgggcgtgatctggggcagcgagaccacctactacaaca | |
| | gcgccctgaagagccggctgaccatcatcaaggacaacagcaag | |
| | agccaggtgttcctgaagatgaacagcctgcagaccgacgacacc | |
| | gccatctactactgcgccaagcactactactacggcggcagctacg | |
| | ccatggactactggggccagggcaccagcgtgaccgtgagcagc | |
| | gaatctaagtacggaccgccctgccccccttgccctatgttctgggt | |
| | gctggtggtggtcggaggcgtgctggcctgctacagcctgctggt | |
| | caccgtggccttcatcatcttttgggtgaaacggggcagaaagaaa | |
| | ctcctgtatatattcaaacaaccatttatgagaccagtacaaactactc | |
| | aagaggaagatggctgtagctgccgatttccagaagaagaagaag | |
| | gaggatgtgaactgcgggtgaagttcagcagaagcgccgacgcc | |
| | cctgcctaccagcagggccagaatcagctgtacaacgagctgaac | |
| | ctgggcagaagggaagagtacgacgtcctggataagcggagag | |
| | gccgggaccctgagatgggcggcaagcctcggeggaagaaccc | |
| | ccaggaaggcctgtataacgaactgcagaaagacaagatggccg | |
| | aggcctacagcgagatcggcatgaagggcgagcggaggcggg | |
| | gcaagggccacgacggcctgtatcagggcctgtccaccgccacc | |
| | aaggatacctacgacgccctgcacatgcaggccctgcccccaag | |
| | g | |
| 34 | MLLLVTSLLLCELPHPAFLLIPDIQMTQTTS | Lisocabtagene |
| | SLSASLGDRVTISCRASQDISKYLNWYQQK | maraleucel CD19 |
| | PDGTVKLLIYHTSRLHSGVPSRFSGSGSGT | CAR amino acid |
| | DYSLTISNLEQEDIATYFCQQGNTLPYTFG | sequence |
| | GGTKLEITGSTSGSGKPGSGEGSTKGEVKL | |
| | QESGPGLVAPSQSLSVTCTVSGVSLPDYGV | |
| | SWIRQPPRKGLEWLGVIWGSETTYYNSAL | |
| | KSRLTIIKDNSKSQVFLKMNSLQTDDTAIY | |
| | YCAKHYYYGGSYAMDYWGQGTSVTVSSE | |
| | SKYGPPCPPCPMFWVLVVVGGVLACYSLL | |
| | VTVAFIIFWVKRGRKKLLYIFKQPFMRPVQ | |
| | TTQEEDGCSCRFPEEEEGGCELRVKFSRSA | |
| | DAPAYQQGQNQLYNELNLGRREEYDVLD | |
| | KRRGRDPEMGGKPRRKNPQEGLYNELQK | |
| | DKMAEAYSEIGMKGERRRGKGHDGLYQG | |
| | LSTATKDTYDALHMQALPPR | |
| 35 | atgcttctcctggtgacaagccttctgctctgtgagttaccacaccca | Axicabtagene |
| | gcattcctcctgatcccagacatccagatgacacagactacatcctc | ciloleucel CD19 |
| | cctgtctgcctctctgggagacagagtcaccatcagttgcagggca | CAR nucleotide |
| | agtcaggacattagtaaatatttaaattggtatcagcagaaaccagat | sequence |
| | ggaactgttaaactcctgatctaccatacatcaagattacactcagg | |
| | agtcccatcaaggttcagtggcagtgggtctggaacagattattctc | |
| | tcaccattagcaacctggagcaagaagatattgccacttacttttgcc | |
| | aacagggtaatacgcttccgtacacgttcggaggggggactaagtt | |
| | ggaaataacaggctccacctctggatccggcaagcccggatctgg | |
| | cgagggatccaccaagggcgaggtgaaactgcaggagtcagga | |
| | cctggcctggtggcgccctcacagagcctgtccgtcacatgcactg | |
| | tctcaggggtctcattacccgactatggtgtaagctggattcgccag | |
| | cctccacgaaagggtctggagtggctgggagtaatatggggtagt | |
| | gaaaccacatactataattcagctctcaaatccagactgaccatcatc | |
| | aaggacaactccaagagccaagttttcttaaaaatgaacagtctgca | |
| | aactgatgacacagccatttactactgtgccaaacattattactacgg | |
| | tggtagctatgctatggactactggggtcaaggaacctcagtcacc | |
| | gtctcctcagcggccgcaattgaagttatgtatcctcctccttaccta | |
| | gacaatgagaagagcaatggaaccattatccatgtgaaagggaaa | |
| | cacctttgtccaagtcccctatttcccggaccttctaagcccttttggg | |
| | tgctggtggtggttgggggagtcctggcttgctatagcttgctagta | |
| | acagtggcctttattattttctgggtgaggagtaagaggagcaggct | |
| | cctgcacagtgactacatgaacatgactccccgccgccccgggcc | |
| | cacccgcaagcattaccagccctatgccccaccacgcgacttcgc | |
| | agcctatcgctccagagtgaagttcagcaggagcgcagacgccc | |
| | ccgcgtaccagcagggccagaaccagctctataacgagctcaatc | |
| | taggacgaagagaggagtacgatgttttggacaagagacgtggcc | |
| | gggaccctgagatggggggaaagccgagaaggaagaaccctca | |
| | ggaaggcctgtacaatgaactgcagaaagataagatggcggagg | |
| | cctacagtgagattgggatgaaaggcgagcgccggaggggcaa | |
| | ggggcacgatggcctttaccagggtctcagtacagccaccaagga | |
| | cacctacgacgcccttcacatgcaggccctgccccctcgc | |
| 36 | MLLLVTSLLLCELPHPAFLLIPDIQMTQTTS | Axicabtagene |
| | SLSASLGDRVTISCRASQDISKYLNWYQQK | ciloleucel CD19 |
| | PDGTVKLLIYHTSRLHSGVPSRFSGSGSGT | CAR amino acid |
| | DYSLTISNLEQEDIATYFCQQGNTLPYTFG | sequence |
| | GGTKLEITGSTSGSGKPGSGEGSTKGEVKL | |
| | QESGPGLVAPSQSLSVTCTVSGVSLPDYGV | |
| | SWIRQPPRKGLEWLGVIWGSETTYYNSAL | |
| | KSRLTIIKDNSKSQVFLKMNSLQTDDTAIY | |
| | YCAKHYYYGGSYAMDYWGQGTSVTVSSA | |
| | AAIEVMYPPPYLDNEKSNGTIIHVKGKHLC | |
| | PSPLFPGPSKPFWVLVVVGGVLACYSLLVT | |
| | VAFIIFWVRSKRSRLLHSDYMNMTPRRPGP | |
| | TRKHYQPYAPPRDFAAYRSRVKFSRSADA | |
| | PAYQQGQNQLYNELNLGRREEYDVLDKR | |
| | RGRDPEMGGKPRRKNPQEGLYNELQKDK | |
| | MAEAYSEIGMKGERRRGKGHDGLYQGLS | |
| | TATKDTYDALHMQALPPR | |

TABLE 8
Annotation of tisagenlecleucel CD19 CAR sequences

| | Nucleotide | Amino Acid |
|---|---|---|
| | Sequence | Sequence |
| Feature | Position | Position |
| CD8α signal peptide | 1-63 | 1-21 |
| FMC63 scFv (V_{L}-3xG₄S linker-V_{H}) | 64-789 | 22-263 |
| CD8α hinge domain | 790-924 | 264-308 |
| CD8α transmembrane domain | 925-996 | 309-332 |
| 4-1BB costimulatory domain | 997-1122 | 333-374 |
| CD3ζ signaling domain | 1123-1458 | 375-486 |

TABLE 9
Annotation of lisocabtagene maraleucel CD19 CAR sequences

| | Nucleotide | Amino Acid |
|---|---|---|
| | Sequence | Sequence |
| Feature | Position | Position |
| GMCSFR-α signal peptide | 1-66 | 1-22 |
| FMC63 scFv (V_{L}-Whitlow linker-V_{H}) | 67-801 | 23-267 |
| IgG4 hinge domain | 802-837 | 268-279 |
| CD28 transmembrane domain | 838-921 | 280-307 |
| 4-1BB costimulatory domain | 922-1047 | 308-349 |
| CD3ζ signaling domain | 1048-1383 | 350-461 |

TABLE 10
Annotation of axicabtagene ciloleucel CD19 CAR sequences

| | Nucleotide | Amino Acid |
|---|---|---|
| | Sequence | Sequence |
| Feature | Position | Position |
| CSF2RA signal peptide | 1-66 | 1-22 |
| FMC63 scFv (V_{L}-Whitlow linker-V_{H}) | 67-801 | 23-267 |
| CD28 hinge domain | 802-927 | 268-309 |
| CD28 transmembrane domain | 928-1008 | 310-336 |
| CD28 costimulatory domain | 1009-1131 | 337-377 |
| CD3ζ signaling domain | 1132-1467 | 378-489 |

In some embodiments, the polycistronic vector comprises an expression cassette that contains a nucleotide sequence encoding CD19 CAR as set forth in SEQ ID NO: 31, 33, or 35, or at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 31, 33, or 35. The encoded CD19 CAR has a corresponding amino acid sequence set forth in SEQ ID NO: 32, 34, or 36, respectively, is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 32, 34, or 36, respectively.

### CD20 CAR

In some embodiments, the CAR is a CD20 CAR, and in these embodiments, the polycistronic vector comprises an expression cassette that contains a nucleotide sequence encoding a CD20 CAR. CD20 is an antigen found on the surface of B cells as early at the pro-B phase and progressively at increasing levels until B cell maturity, as well as on the cells of most B-cell neoplasms. CD20 positive cells are also sometimes found in cases of Hodgkins disease, myeloma, and thymoma. In some embodiments, the CD20 CAR may comprise a signal peptide, an extracellular binding domain that specifically binds CD20, a hinge domain, a transmembrane domain, an intracellular costimulatory domain, and/or an intracellular signaling domain in tandem.

In some embodiments, the signal peptide of the CD20 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Leu16, IF5, 1.5.3, rituximab, obinutuzumab, ibritumomab, ofatumumab, tositumumab, odronextamab, veltuzumab, ublituximab, and ocrelizumab. In any of these embodiments, the extracellular binding domain of the CD20 CAR can comprise or consist of the V_{H}, the V_{L}, and/or one or more CDRs of any of the antibodies.

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14(12):1025-1033 (2001). In some embodiments, the linker is a 3×G₄S linker. In other embodiments, the linker is a Whitlow linker as described herein. In some embodiments, the amino acid sequences of different portions of the entire Leu16-derived scFv (also referred to as Leu16 scFv) and its different portions are provided in Table 11 below. In some embodiments, the CD20-specific scFv comprises or consists of an amino acid sequence set forth in SEQ ID NO:37, 38, or 42, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:37, 38, or 42. In some embodiments, the CD20-specific scFv may comprise one or more CDRs having amino acid sequences set forth in SEQ ID NOs: 39-41, 43 and 44. In some embodiments, the CD20-specific scFv may comprise a light chain with one or more CDRs having amino acid sequences set forth in SEQ ID NOs: 39-41. In some embodiments, the CD20-specific scFv may comprise a heavy chain with one or more CDRs having amino acid sequences set forth in SEQ ID NOs: 43-44. In any of these embodiments, the CD20-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD20 CAR comprises or consists of the one or more CDRs as described herein.

TABLE 11
Exemplary sequences of anti-CD20
scFv and components

| SEQ | | |
|---|---|---|
| ID NO: | Amino Acid Sequence | Description |
| 37 | DIVLTQSPAILSASPGEKVTMTCRAS | Anti-CD20 Leu16 |
| | SSVNYMDWYQKKPGSSPKPWIYAT | scFv entire |
| | SNLASGVPARFSGSGSGTSYSLTISR | sequence, with |
| | VEAEDAATYYCQQWSFNPPTFGGG | Whitlow linker |
| | TKLEIKGSTSGSGKPGSGEGSTKGEV | |
| | QLQQSGAELVKPGASVKMSCKASG | |
| | YTFTSYNMHWVKQTPGQGLEWIGA | |
| | IYPGNGDTSYNQKFKGKATLTADKS | |
| | SSTAYMQLSSLTSEDSADYYCARSN | |
| | YYGSSYWFFDVWGAGTTVTVSS | |
| 38 | DIVLTQSPAILSASPGEKVTMTCRAS | Anti-CD20 Leu16 |
| | SSVNYMDWYQKKPGSSPKPWIYAT | scFv light chain |
| | SNLASGVPARFSGSGSGTSYSLTISR | variable region |
| | VEAEDAATYYCQQWSFNPPTFGGG | |
| | TKLEIK | |
| 39 | RASSSVNYMD | Anti-CD20 Leu16 |
| | | scFv light |
| | | chain CDR1 |
| 40 | ATSNLAS | Anti-CD20 Leu16 |
| | | scFv light |
| | | chain CDR2 |
| 41 | QQWSFNPPT | Anti-CD20 Leu16 |
| | | scFv light |
| | | chain CDR3 |
| 42 | EVQLQQSGAELVKPGASVKMSCKA | Anti-CD20 Leu16 |
| | SGYTFTSYNMHWVKQTPGQGLEWI | scFv heavy |
| | GAIYPGNGDTSYNQKFKGKATLTA | chain |
| | DKSSSTAYMQLSSLTSEDSADYYCA | |
| | RSNYYGSSYWFFDVWGAGTTVTVS | |
| | S | |
| 43 | SYNMH | Anti-CD20 Leu16 |
| | | scFv heavy |
| | | chain CDR1 |
| 44 | AIYPGNGDTSYNQKFKG | Anti-CD20 Leu16 |
| | | scFv heavy |
| | | chain CDR2 |

In some embodiments, the hinge domain of the CD20 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO: 10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO: 12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO: 13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the CD20 CAR comprises a CD8α transmembrane domain, for example, a human CD8α transmembrane domain. In some embodiments, the CD8α transmembrane domain comprises or consists of an amino acid sequence set forth in SEQ ID NO: 14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD20 CAR comprises a 4-1BB costimulatory domain, for example, a human 4-1BB costimulatory domain. In some embodiments, the 4-1BB costimulatory domain comprises or consists of an amino acid sequence set forth in SEQ ID NO: 16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the CD20 CAR comprises a CD3 zeta (ζ) signaling domain, for example, a human CD3ζ signaling domain. In some embodiments, the CD3ζ signaling domain comprises or consists of an amino acid sequence set forth in SEQ ID NO: 18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 18.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD8α hinge domain of SEQ ID NO:9, the CD8α transmembrane domain of SEQ ID NO: 14, the 4-1BB costimulatory domain of SEQ ID NO: 16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD28 hinge domain of SEQ ID NO: 10, the CD8α transmembrane domain of SEQ ID NO:14, the 4-1BB costimulatory domain of SEQ ID NO: 16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the IgG4 hinge domain of SEQ ID NO:11 or SEQ ID NO: 12, the CD8α transmembrane domain of SEQ ID NO: 14, the 4-1BB costimulatory domain of SEQ ID NO: 16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD8α hinge domain of SEQ ID NO:9, the CD28 transmembrane domain of SEQ ID NO: 15, the 4-1BB costimulatory domain of SEQ ID NO:16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD28 hinge domain of SEQ ID NO: 10, the CD28 transmembrane domain of SEQ ID NO: 15, the 4-1BB costimulatory domain of SEQ ID NO: 16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the IgG4 hinge domain of SEQ ID NO:11 or SEQ ID NO: 1, the CD28 transmembrane domain of SEQ ID NO: 15, the 4-1BB costimulatory domain of SEQ ID NO:16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### CD22 CAR

In some embodiments, the CAR is a CD22 CAR, and in these embodiments, the polycistronic vector comprises an expression cassette that contains a nucleotide sequence encoding a CD22 CAR. CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise a signal peptide, an extracellular binding domain that specifically binds CD22, a hinge domain, a transmembrane domain, an intracellular costimulatory domain, and/or an intracellular signaling domain in tandem.

In some embodiments, the signal peptide of the CD22 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, and pinatuzumab. In any of these embodiments, the extracellular binding domain of the CD22 CAR can comprise or consist of the V_{H}, the V_{L}, and/or one or more CDRs of any of the antibodies.

In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a 3×G₄S linker. In other embodiments, the Whitlow linker may be used instead. In some embodiments, the amino acid sequences of the entire m971-derived scFv (also referred to as m971 scFv) and its different portions are provided in Table 12 below. In some embodiments, the CD22-specific scFv comprises or consists of an amino acid sequence set forth in SEQ ID NO:45, 46, or 50, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:45, 46, or 50. In some embodiments, the CD22-specific scFv may comprise one or more CDRs having amino acid sequences set forth in SEQ ID NOs: 47-49 and 51-53. In some embodiments, the CD22-specific scFv may comprise a heavy chain with one or more CDRs having amino acid sequences set forth in SEQ ID NOs: 47-49. In some embodiments, the CD22-specific scFv may comprise a light chain with one or more CDRs having amino acid sequences set forth in SEQ ID NOs: 51-53. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 pM). In some embodiments, the scFv derived from m971-L7 comprises the V_{H} and the V_{L} of m971-L7 connected by a 3×G₄S linker. In other embodiments, the Whitlow linker may be used instead. In some embodiments, the amino acid sequences of the entire m971-L7-derived scFv (also referred to as m971-L7 scFv) and its different portions are provided in Table 12 below. In some embodiments, the CD22-specific scFv comprises or consists of an amino acid sequence set forth in SEQ ID NO:54, 55, or 59, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:54, 55, or 59. In some embodiments, the CD22-specific scFv may comprise one or more CDRs having amino acid sequences set forth in SEQ ID NOs: 56-58 and 60-62. In some embodiments, the CD22-specific scFv may comprise a heavy chain with one or more CDRs having amino acid sequences set forth in SEQ ID NOs: 56-58. In some embodiments, the CD22-specific scFv may comprise a light chain with one or more CDRs having amino acid sequences set forth in SEQ ID NOs: 60-62. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

TABLE 12
Exemplary sequences of anti-CD22 scFv and components

| SEQ ID NO: | Amino Acid Sequence | Description |
|---|---|---|
| 45 | QVQLQQSGPGLVKPSQTLSLTCAISG | Anti-CD22 m971 scFv |
| | DSVSSNSAAWNWIRQSPSRGLEWL | entire sequence, with |
| | GRTYYRSKWYNDYAVSVKSRITINP | 3xG4S linker |
| | DTSKNQFSLQLNSVTPEDTAVYYCA | |
| | REVTGDLEDAFDIWGQGTMVTVSS | |
| | GGGGSGGGGSGGGGSDIQMTQSPSS | |
| | LSASVGDRVTITCRASQTIWSYLNW | |
| | YQQRPGKAPNLLIYAASSLQSGVPS | |
| | RFSGRGSGTDFTLTISSLQAEDFATY | |
| | YCQQSYSIPQTFGQGTKLEIK | |
| 46 | QVQLQQSGPGLVKPSQTLSLTCAISG | Anti-CD22 m971 scFv |
| | DSVSSNSAAWNWIRQSPSRGLEWL | heavy chain variable |
| | GRTYYRSKWYNDYAVSVKSRITINP | region |
| | DTSKNQFSLQLNSVTPEDTAVYYCA | |
| | REVTGDLEDAFDIWGQGTMVTVSS | |
| 47 | GDSVSSNSAA | Anti-CD22 m971 scFv |
| | | heavy chain CDR1 |
| 48 | TYYRSKWYN | Anti-CD22 m971 scFv |
| | | heavy chain CDR2 |
| 49 | AREVTGDLEDAFDI | Anti-CD22 m971 scFv |
| | | heavy chain CDR3 |
| 50 | DIQMTQSPSSLSASVGDRVTITCRAS | Anti-CD22 m971 scFv |
| | QTIWSYLNWYQQRPGKAPNLLIYA | light chain |
| | ASSLQSGVPSRFSGRGSGTDFTLTISS | |
| | LQAEDFATYYCQQSYSIPQTFGQGT | |
| | KLEIK | |
| 51 | QTIWSY | Anti-CD22 m971 scFv |
| | | light chain CDR1 |
| 52 | AAS | Anti-CD22 m971 scFv |
| | | light chain CDR2 |
| 53 | QQSYSIPQT | Anti-CD22 m971 scFv |
| | | light chain CDR3 |
| 54 | QVQLQQSGPGMVKPSQTLSLTCAIS | Anti-CD22 m971-L7 |
| | GDSVSSNSVAWNWIRQSPSRGLEW | scFv entire sequence, |
| | LGRTYYRSTWYNDYAVSMKSRITIN | with 3xG4S linker |
| | PDTNKNQFSLQLNSVTPEDTAVYYC | |
| | AREVTGDLEDAFDIWGQGTMVTVS | |
| | SGGGGSGGGGSGGGGSDIQMIQSPS | |
| | SLSASVGDRVTITCRASQTIWSYLN | |
| | WYRQRPGEAPNLLIYAASSLQSGVP | |
| | SRFSGRGSGTDFTLTISSLQAEDFAT | |
| | YYCQQSYSIPQTFGQGTKLEIK | |
| 55 | QVQLQQSGPGMVKPSQTLSLTCAIS | Anti-CD22 m971-L7 |
| | GDSVSSNSVAWNWIRQSPSRGLEW | scFv heavy chain |
| | LGRTYYRSTWYNDYAVSMKSRITIN | variable region |
| | PDTNKNQFSLQLNSVTPEDTAVYYC | |
| | AREVTGDLEDAFDIWGQGTMVTVS | |
| | S | |
| 56 | GDSVSSNSVA | Anti-CD22 m971-L7 |
| | | scFv heavy chain CDR1 |
| 57 | TYYRSTWYN | Anti-CD22 m971-L7 |
| | | scFv heavy chain CDR2 |
| 58 | AREVTGDLEDAFDI | Anti-CD22 m971-L7 |
| | | scFv heavy chain CDR3 |
| 59 | DIQMIQSPSSLSASVGDRVTITCRAS | Anti-CD22 m971-L7 |
| | QTIWSYLNWYRQRPGEAPNLLIYAA | scFv light chain variable |
| | SSLQSGVPSRFSGRGSGTDFTLTISSL | region |
| | QAEDFATYYCQQSYSIPQTFGQGTK | |
| | LEIK | |
| 60 | QTIWSY | Anti-CD22 m971-L7 |
| | | scFv light chain CDR1 |
| 61 | AAS | Anti-CD22 m971-L7 |
| | | scFv light chain CDR2 |
| 62 | QQSYSIPQT | Anti-CD22 m971-L7 |
| | | scFv light chain CDR3 |

In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280(1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO: 10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO: 11 or SEQ ID NO: 12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO: 13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the CD22 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 15.

In some embodiments, the intracellular costimulatory domain of the CD22 CAR comprises a 4-1BB costimulatory domain, for example, a human 4-1 BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the CD22 CAR comprises a CD3 zeta (ζ) signaling domain, for example, a human CD3ζ signaling domain. In some embodiments, the CD3ζ signaling domain comprises or consists of an amino acid sequence set forth in SEQ ID NO: 18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD8α hinge domain of SEQ ID NO:9, the CD8α transmembrane domain of SEQ ID NO: 14, the 4-1BB costimulatory domain of SEQ ID NO:16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD28 hinge domain of SEQ ID NO: 10, the CD8α transmembrane domain of SEQ ID NO: 14, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the IgG4 hinge domain of SEQ ID NO: 11 or SEQ ID NO:12, the CD8α transmembrane domain of SEQ ID NO: 14, the 4-1BB costimulatory domain of SEQ ID NO: 16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD8α hinge domain of SEQ ID NO:9, the CD28 transmembrane domain of SEQ ID NO:15, the 4-1BB costimulatory domain of SEQ ID NO: 16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD28 hinge domain of SEQ ID NO: 10, the CD28 transmembrane domain of SEQ ID NO: 15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the IgG4 hinge domain of SEQ ID NO: 11 or SEQ ID NO: 12, the CD28 transmembrane domain of SEQ ID NO: 15, the 4-1BB costimulatory domain of SEQ ID NO:16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### BCMA CAR

In some embodiments, the CAR is a BCMA CAR, and in these embodiments, the polycistronic vector comprises an expression cassette that contains a nucleotide sequence encoding a BCMA CAR. BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR may comprise a signal peptide, an extracellular binding domain that specifically binds BCMA, a hinge domain, a transmembrane domain, an intracellular costimulatory domain, and/or an intracellular signaling domain in tandem.

In some embodiments, the signal peptide of the BCMA CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, erlanatamab, teclistamab, LCAR-B38M, and ciltacabtagene. In any of these embodiments, the extracellular binding domain of the BCMA CAR can comprise or consist of the V_{H}, the V_{L}, and/or one or more CDRs of any of the antibodies.

In some embodiments, the extracellular binding domain of the BCMA CAR comprises an scFv derived from C11D5.3, a murine monoclonal antibody as described in Carpenter et al., Clin. Cancer Res. 19(8):2048-2060 (2013). See also PCT Application Publication No. WO2010/104949. The C11D5.3-derived scFv may comprise the heavy chain variable region (V_{H}) and the light chain variable region (V_{L}) of C11D5.3 connected by the Whitlow linker, the amino acid sequences of which is provided in Table 13 below. In some embodiments, the BCMA-specific extracellular binding domain comprises or consists of an amino acid sequence set forth in SEQ ID NO:63, 64, or 68, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:63, 64, or 68. In some embodiments, the BCMA-specific extracellular binding domain may comprise one or more CDRs having amino acid sequences set forth in SEQ ID NOs: 65-67 and 69-71. In some embodiments, the BCMA-specific extracellular binding domain may comprise a light chain with one or more CDRs having amino acid sequences set forth in SEQ ID NOs: 65-67. In some embodiments, the BCMA-specific extracellular binding domain may comprise a heavy chain with one or more CDRs having amino acid sequences set forth in SEQ ID NOs: 69-71. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from another murine monoclonal antibody, C12A3.2, as described in Carpenter et al., Clin. Cancer Res. 19(8):2048-2060 (2013) and PCT Application Publication No. WO2010/104949, the amino acid sequence of which is also provided in Table 13 below. In some embodiments, the BCMA-specific extracellular binding domain comprises or consists of an amino acid sequence set forth in SEQ ID NO:72, 73, or 77, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:72, 73, or 77. In some embodiments, the BCMA-specific extracellular binding domain may comprise one or more CDRs having amino acid sequences set forth in SEQ ID NOs: 74-76 and 78-80. In some embodiments, the BCMA-specific extracellular binding domain may comprise a light chain with one or more CDRs having amino acid sequences set forth in SEQ ID NOs: 74-76. In some embodiments, the BCMA-specific extracellular binding domain may comprise a heavy chain with one or more CDRs having amino acid sequences set forth in SEQ ID NOs: 78-80. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29(5):585-601 (2018)). See also, PCT Application Publication No. WO2012163805.

In some embodiments, the extracellular binding domain of the BCMA CAR comprises single variable fragments of two heavy chains (VHH) that can bind to two epitopes of BCMA as described in Zhao et al., J. Hematol. Oncol. 11(1): 141 (2018), also referred to as LCAR-B38M. See also, PCT Application Publication No. WO2018/028647.

In some embodiments, the extracellular binding domain of the BCMA CAR comprises a fully human heavy-chain variable domain (FHVH) as described in Lam et al., Nat. Commun. 11(1):283 (2020), also referred to as FHVH33. See also, PCT Application Publication No. WO2019/006072. The amino acid sequences of FHVH33 and its CDRs are provided in Table 13 below. In some embodiments, the BCMA-specific extracellular binding domain comprises or consists of an amino acid sequence set forth in SEQ ID NO:81 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:81. In some embodiments, the BCMA-specific extracellular binding domain may comprise one or more CDRs having amino acid sequences set forth in SEQ ID NOs: 82-84. In any of these embodiments, the BCMA-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from CT103A (or CAR0085) as described in U.S. Pat. No. 11,026,975 B2, the amino acid sequence of which is provided in Table 13 below. In some embodiments, the BCMA-specific extracellular binding domain comprises or consists of an amino acid sequence set forth in SEQ ID NO:118, 119, or 123, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 118, 119, or 123. In some embodiments, the BCMA-specific extracellular binding domain may comprise one or more CDRs having amino acid sequences set forth in SEQ ID NOs: 120-122 and 124-126. In some embodiments, the BCMA-specific extracellular binding domain may comprise a light chain with one or more CDRs having amino acid sequences set forth in SEQ ID NOs: 120-122. In some embodiments, the BCMA-specific extracellular binding domain may comprise a heavy chain with one or more CDRs having amino acid sequences set forth in SEQ ID NOs: 124-126. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

Additionally, CARs and binders directed to BCMA have been described in U.S. Application Publication Nos. 2020/0246381 A1 and 2020/0339699 A1, the entire contents of each of which are incorporated by reference herein.

TABLE 13
Exemplary sequences of anti-BCMA binder and components

| SEQ ID NO: | Amino Acid Sequence | Description |
|---|---|---|
| 63 | DIVLTQSPASLAMSLGKRATISCRAS | Anti-BCMA C11D5.3 |
| | ESVSVIGAHLIHWYQQKPGQPPKLLI | scFv entire sequence, |
| | YLASNLETGVPARFSGSGSGTDFTLT | with Whitlow linker |
| | IDPVEEDDVAIYSCLQSRIFPRTFGG | |
| | GTKLEIKGSTSGSGKPGSGEGSTKG | |
| | QIQLVQSGPELKKPGETVKISCKASG | |
| | YTFTDYSINWVKRAPGKGLKWMG | |
| | WINTETREPAYAYDFRGRFAFSLETS | |
| | ASTAYLQINNLKYEDTATYFCALDY | |
| | SYAMDYWGQGTSVTVSS | |
| 64 | DIVLTQSPASLAMSLGKRATISCRAS | Anti-BCMA C11D5.3 |
| | ESVSVIGAHLIHWYQQKPGQPPKLLI | scFv light chain variable |
| | YLASNLETGVPARFSGSGSGTDFTLT | region |
| | IDPVEEDDVAIYSCLQSRIFPRTFGG | |
| | GTKLEIK | |
| 65 | RASESVSVIGAHLIH | Anti-BCMA C11D5.3 |
| | | scFv light chain CDR1 |
| 66 | LASNLET | Anti-BCMA C11D5.3 |
| | | scFv light chain CDR2 |
| 67 | LQSRIFPRT | Anti-BCMA C11D5.3 |
| | | scFv light chain CDR3 |
| 68 | QIQLVQSGPELKKPGETVKISCKASG | Anti-BCMA C11D5.3 |
| | YTFTDYSINWVKRAPGKGLKWMG | scFv heavy chain |
| | WINTETREPAYAYDFRGRFAFSLETS | variable region |
| | ASTAYLQINNLKYEDTATYFCALDY | |
| | SYAMDYWGQGTSVTVSS | |
| 69 | DYSIN | Anti-BCMA C11D5.3 |
| | | scFv heavy chain CDR1 |
| 70 | WINTETREPAYAYDFRG | Anti-BCMA C11D5.3 |
| | | scFv heavy chain CDR2 |
| 71 | DYSYAMDY | Anti-BCMA C11D5.3 |
| | | scFv heavy chain CDR3 |
| 72 | DIVLTQSPPSLAMSLGKRATISCRAS | Anti-BCMA C12A3.2 |
| | ESVTILGSHLIYWYQQKPGQPPTLLI | scFv entire sequence, |
| | QLASNVQTGVPARFSGSGSRTDFTL | with Whitlow linker |
| | TIDPVEEDDVAVYYCLQSRTIPRTFG | |
| | GGTKLEIKGSTSGSGKPGSGEGSTK | |
| | GQIQLVQSGPELKKPGETVKISCKAS | |
| | GYTFRHYSMNWVKQAPGKGLKWM | |
| | GRINTESGVPIYADDFKGRFAFSVET | |
| | SASTAYLVINNLKDEDTASYFCSND | |
| | YLYSLDFWGQGTALTVSS | |
| 73 | DIVLTQSPPSLAMSLGKRATISCRAS | Anti-BCMA C12A3.2 |
| | ESVTILGSHLIYWYQQKPGQPPTLLI | scFv light chain variable |
| | QLASNVQTGVPARFSGSGSRTDFTL | region |
| | TIDPVEEDDVAVYYCLQSRTIPRTFG | |
| | GGTKLEIK | |
| 74 | RASESVTILGSHLIY | Anti-BCMA C12A3.2 |
| | | scFv light chain CDR1 |
| 75 | LASNVQT | Anti-BCMA C12A3.2 |
| | | scFv light chain CDR2 |
| 76 | LQSRTIPRT | Anti-BCMA C12A3.2 |
| | | scFv light chain CDR3 |
| 77 | QIQLVQSGPELKKPGETVKISCKASG | Anti-BCMA C12A3.2 |
| | YTFRHYSMNWVKQAPGKGLKWMG | scFv heavy chain |
| | RINTESGVPIYADDFKGRFAFSVETS | variable region |
| | ASTAYLVINNLKDEDTASYFCSNDY | |
| | LYSLDFWGQGTALTVSS | |
| 78 | HYSMN | Anti-BCMA C12A3.2 |
| | | scFv heavy chain CDR1 |
| 79 | RINTESGVPIYADDFKG | Anti-BCMA C12A3.2 |
| | | scFv heavy chain CDR2 |
| 80 | DYLYSLDF | Anti-BCMA C12A3.2 |
| | | scFv heavy chain CDR3 |
| 81 | EVQLLESGGGLVQPGGSLRLSCAAS | Anti-BCMA FHVH33 |
| | GFTFSSYAMSWVRQAPGKGLEWVS | entire sequence |
| | SISGSGDYIYYADSVKGRFTISRDISK | |
| | NTLYLQMNSLRAEDTAVYYCAKEG | |
| | TGANSSLADYRGQGTLVTVSS | |
| 82 | GFTFSSYA | Anti-BCMA FHVH33 |
| | | CDR1 |
| 83 | ISGSGDYI | Anti-BCMA FHVH33 |
| | | CDR2 |
| 84 | AKEGTGANSSLADY | Anti-BCMA FHVH33 |
| | | CDR3 |
| 118 | DIQMTQSPSSLSASVGDRVTITCRAS | Anti-BCMA CT103A |
| | QSISSYLNWYQQKPGKAPKLLIYAA | scFv entire sequence, |
| | SSLQSGVPSRFSGSGSGTDFTLTISSL | with Whitlow linker |
| | QPEDFATYYCQQKYDLLTFGGGTK | |
| | VEIKGSTSGSGKPGSGEGSTKGQLQ | |
| | LQESGPGLVKPSETLSLTCTVSGGSI | |
| | SSSSYYWGWIRQPPGKGLEWIGSISY | |
| | SGSTYYNPSLKSRVTISVDTSKNQFS | |
| | LKLSSVTAADTAVYYCARDRGDTIL | |
| | DVWGQGTMVTVSS | |
| 119 | DIQMTQSPSSLSASVGDRVTITCRAS | Anti-BCMA CT103A |
| | QSISSYLNWYQQKPGKAPKLLIYAA | scFv light chain variable |
| | SSLQSGVPSRFSGSGSGTDFTLTISSL | region |
| | QPEDFATYYCQQKYDLLTFGGGTK | |
| | VEIK | |
| 120 | QSISSY | Anti-BCMA CT103A |
| | | scFv light chain CDR1 |
| 121 | AAS | Anti-BCMA CT103A |
| | | scFv light chain CDR2 |
| 122 | QQKYDLLT | Anti-BCMA CT103A |
| | | scFv light chain CDR3 |
| 123 | QLQLQESGPGLVKPSETLSLTCTVSG | Anti-BCMA CT103A |
| | GSISSSSYYWGWIRQPPGKGLEWIGS | scFv heavy chain |
| | ISYSGSTYYNPSLKSRVTISVDTSKN | variable region |
| | QFSLKLSSVTAADTAVYYCARDRG | |
| | DTILDVWGQGTMVTVSS | |
| 124 | GGSISSSSYY | Anti-BCMA CT103A |
| | | scFv heavy chain CDR1 |
| 125 | ISYSGST | Anti-BCMA CT103A |
| | | scFv heavy chain CDR2 |
| 126 | ARDRGDTILDV | Anti-BCMA CT103A |
| | | scFv heavy chain CDR3 |

In some embodiments, the hinge domain of the BCMA CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 13.

In some embodiments, the transmembrane domain of the BCMA CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the BCMA CAR comprises a 4-1BB costimulatory domain, for example, a human 4-1BB costimulatory domain. In some embodiments, the 4-1BB costimulatory domain comprises or consists of an amino acid sequence set forth in SEQ ID NO: 16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the BCMA CAR comprises a CD3 zeta (ζ) signaling domain, for example, a human CD3ζ signaling domain. In some embodiments, the CD3ζ signaling domain comprises or consists of an amino acid sequence set forth in SEQ ID NO: 18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:9, the CD8α transmembrane domain of SEQ ID NO:14, the 4-1BB costimulatory domain of SEQ ID NO: 16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide (e.g., a CD8α signal peptide) as described.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:9, the CD8α transmembrane domain of SEQ ID NO:14, the CD28 costimulatory domain of SEQ ID NO:17, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide as described.

In some embodiments, the polycistronic vector comprises an expression cassette that contains a nucleotide sequence encoding a BCMA CAR as set forth in SEQ ID NO:127 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 127 (see Table 14). The encoded BCMA CAR has a corresponding amino acid sequence set forth in SEQ ID NO: 128 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:128, with the following components; CD8α signal peptide, CT103A scFv (V_{L}-Whitlow linker-V_{H}), CD8α hinge domain, CD8α transmembrane domain, 4-1BB costimulatory domain, and CD3ζ signaling domain.

In some embodiments, the polycistronic vector comprises an expression cassette that contains a nucleotide sequence encoding a commercially available embodiment of BCMA CAR, including, for example, idecabtagene vicleucel (ide-cel, also called bb2121). In some embodiments, the polycistronic vector comprises an expression cassette that contains a nucleotide sequence encoding idecabtagene vicleucel or portions thereof. Idecabtagene vicleucel comprises a BCMA CAR with the following components: the BB2121 binder, CD8α hinge domain, CD8α transmembrane domain, 4-1BB costimulatory domain, and CD3ζ signaling domain.

TABLE 14
Exemplary sequences of BCMA CARs

| SEQ ID NO: | Sequence | Description |
|---|---|---|
| 127 | atggccttaccagtgaccgccttgctcctgccgctggccttgctgc | Exemplary BCMA |
| | tccacgccgccaggccggacatccagatgacccagtctccatcct | CAR nucleotide |
| | ccctgtctgcatctgtaggagacagagtcaccatcacttgccggg | sequence |
| | caagtcagagcattagcagctatttaaattggtatcagcagaaacc | |
| | agggaaagcccctaagctcctgatctatgctgcatccagtttgcaa | |
| | agtggggtcccatcaaggttcagtggcagtggatctgggacagat | |
| | ttcactctcaccatcagcagtctgcaacctgaagattttgcaacttac | |
| | tactgtcagcaaaaatacgacctcctcacttttggcggagggacca | |
| | aggttgagatcaaaggcagcaccagcggctccggcaagcctgg | |
| | ctctggcgagggcagcacaaagggacagctgcagctgcagga | |
| | gtcgggcccaggactggtgaagccttcggagaccctgtccctca | |
| | cctgcactgtctctggtggctccatcagcagtagtagttactactgg | |
| | ggctggatccgccagcccccagggaaggggctggagtggattg | |
| | ggagtatctcctatagtgggagcacctactacaacccgtccctcaa | |
| | gagtcgagtcaccatatccgtagacacgtccaagaaccagttctc | |
| | cctgaagctgagttctgtgaccgccgcagacacggcggtgtacta | |
| | ctgcgccagagatcgtggagacaccatactagacgtatggggtc | |
| | agggtacaatggtcaccgtcagctcattcgtgcccgtgttcctgcc | |
| | cgccaaacctaccaccacccctgcccctagacctcccaccccag | |
| | ccccaacaatcgccagccagcctctgtctctgcggcccgaagcct | |
| | gtagacctgctgccggcggagccgtgcacaccagaggcctgga | |
| | cttcgcctgcgacatctacatctgggcccctctggccggcacctgt | |
| | ggcgtgctgctgctgagcctggtgatcaccctgtactgcaaccac | |
| | cggaacaaacggggcagaaagaaactcctgtatatattcaaacaa | |
| | ccatttatgagaccagtacaaactactcaagaggaagatggctgta | |
| | gctgccgatttccagaagaagaagaaggaggatgtgaactgaga | |
| | gtgaagttcagcagatccgccgacgcccctgcctaccagcaggg | |
| | acagaaccagctgtacaacgagctgaacctgggcagacgggaa | |
| | gagtacgacgtgctggacaagcggagaggccgggaccccgag | |
| | atgggcggaaagcccagacggaagaacccccaggaaggcctg | |
| | tataacgaactgcagaaagacaagatggccgaggcctacagcg | |
| | agatcggcatgaagggcgagcggaggcgcggcaagggccac | |
| | gatggcctgtaccagggcctgagcaccgccaccaaggacacct | |
| | acgacgccctgcacatgcaggccctgccccccaga | |
| 128 | MALPVTALLLPLALLLHAARPDIQMTQSP | Exemplary BCMA |
| | SSLSASVGDRVTITCRASQSISSYLNWYQQ | CAR amino acid |
| | KPGKAPKLLIYAASSLQSGVPSRFSGSGSG | sequence |
| | TDFTLTISSLQPEDFATYYCQQKYDLLTFG | |
| | GGTKVEIKGSTSGSGKPGSGEGSTKGQLQ | |
| | LQESGPGLVKPSETLSLTCTVSGGSISSSSY | |
| | YWGWIRQPPGKGLEWIGSISYSGSTYYNP | |
| | SLKSRVTISVDTSKNQFSLKLSSVTAADTA | |
| | VYYCARDRGDTILDVWGQGTMVTVSSFV | |
| | PVFLPAKPTTTPAPRPPTPAPTIASQPLSLR | |
| | PEACRPAAGGAVHTRGLDFACDIYIWAPL | |
| | AGTCGVLLLSLVITLYCNHRNKRGRKKLL | |
| | YIFKQPFMRPVQTTQEEDGCSCRFPEEEEG | |
| | GCELRVKFSRSADAPAYQQGQNQLYNEL | |
| | NLGRREEYDVLDKRRGRDPEMGGKPRRK | |
| | NPQEGLYNELQKDKMAEAYSEIGMKGER | |
| | RRGKGHDGLYQGLSTATKDTYDALHMQ | |
| | ALPPR | |

### K. Overexpression of Tolerogenic Factors

For all of these technologies, well known recombinant techniques are used, to generate recombinant nucleic acids as outlined herein. In certain embodiments, the recombinant nucleic acids encoding a tolerogenic factor may be operably linked to one or more regulatory nucleotide sequences in an expression construct. Regulatory nucleotide sequences will generally be appropriate for the host cell and recipient subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector's copy number, the ability to control that copy number, or the expression of any other protein encoded by the vector, such as antibiotic markers.

Examples of suitable mammalian promoters include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication (Fiers et al., Nature 273: 113-120 (1978)). The immediate early promoter of the human cytomegalovirus is conveniently obtained as a HindIII E restriction fragment (Greenaway et al., Gene 18: 355-360 (1982)). The foregoing references are incorporated by reference in their entirety.

The process of introducing the polynucleotides described herein into cells can be achieved by any suitable technique. Suitable techniques include calcium phosphate or lipid-mediated transfection, electroporation, and transduction or infection using a viral vector. In some embodiments, the polynucleotides are introduced into a cell via viral transduction (e.g., lentiviral transduction).

Once altered, the presence of expression of any of the molecule described herein can be assayed using known techniques, such as Western blots, ELISA assays, FACS assays, and the like.

In some embodiments, the present technology provides hypoimmunogenic T cells that comprise a “suicide gene” or “suicide switch”. These are incorporated to function as a “safety switch” that can cause the death of the hypoimmunogenic T cells should they grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene and the trigger is ganciclovir. In other embodiments, the suicide gene is the Escherichia coli cytosine deaminase (EC-CD) gene and the trigger is 5-fluorocytosine (5-FC) (Barese et al., Mol. Therap. 20(10): 1932-1943 (2012), Xu et al., Cell Res. 8:73-8 (1998), both incorporated herein by reference in their entirety.)

In other embodiments, the suicide gene is an inducible Caspase protein. An inducible Caspase protein comprises at least a portion of a Caspase protein capable of inducing apoptosis. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, AP1903. Thus, the suicide function of iCasp9 is triggered by the administration of a chemical inducer of dimerization (CID). In some embodiments, the CID is the small molecule drug API 903. Dimerization causes the rapid induction of apoptosis. (See WO2011146862; Stasi et al., N. Engl. J. Med 365:18 (2011); Tey et al., Biol. Blood Marrow Transplant. 13:913-924 (2007), each of which are incorporated by reference herein in their entirety.)

### L. Methods of Genetic Modifications

The process of introducing the polynucleotides described herein into cells can be achieved by any suitable technique. Suitable techniques include calcium phosphate or lipid-mediated transfection, electroporation, fusogens, and transduction or infection using a viral vector. In some embodiments, the polynucleotides are introduced into a cell via viral transduction (e.g., lentiviral transduction) or otherwise delivered on a viral vector (e.g., fusogen-mediated delivery). The polynucleotides described herein can be introduced into cells in vitro, ex vivo from a donor subject, or in vivo in a recipient patient.

Unlike certain methods of introducing the polynucleotides described herein into cells which generally involve activating cells, such as activating T cells (e.g., CD8⁺ T cells), suitable techniques can be utilized to introduce polynucleotides into non-activated T cells. Suitable techniques include, but are not limited to, activation of T cells, such as CD8⁺ T cells, with one or more antibodies which bind to CD3, CD8, and/or CD28, or fragments or portions thereof (e.g., scFv and VHH) that may or may not be bound to beads. Other suitable techniques include, but are not limited to, fusogen-mediated introduction of polynucleotides into T cells in non-activated T cells (e.g., CD8⁺ T cells) that have not been previously contacted with one or more activating antibodies or fragments or portions thereof (e.g., CD3, CD8, and/or CD28). In some embodiments, fusogen-mediated introduction of polynucleotides into T cells is performed in vivo in a patient (e.g., after the T cells have been administered to a recipient patient). In other embodiments, fusogen-mediated introduction of polynucleotides into T cells is performed in vivo in a subject (e.g., before the cells have been isolated from the donor subject.

In some embodiments, a rare-cutting endonuclease is introduced into a cell containing the target polynucleotide sequence in the form of a nucleic acid encoding a rare-cutting endonuclease. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

The present technology contemplates altering target polynucleotide sequences in any manner which is available to the skilled artisan utilizing a CRISPR/Cas system. Any CRISPR/Cas system that is capable of altering a target polynucleotide sequence in a cell can be used. Such CRISPR-Cas systems can employ a variety of Cas proteins (Haft et al. PLOS Comput Biol. 2005; 1(6)e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system.

The CRISPR/Cas systems can be used to alter any target polynucleotide sequence in a cell. Those skilled in the art will readily appreciate that desirable target polynucleotide sequences to be altered in any particular cell may correspond to any genomic sequence for which expression of the genomic sequence is associated with a disorder or otherwise facilitates entry of a pathogen into the cell. For example, a desirable target polynucleotide sequence to alter in a cell may be a polynucleotide sequence corresponding to a genomic sequence which contains a disease associated single polynucleotide polymorphism. In such example, the CRISPR/Cas systems can be used to correct the disease associated SNP in a cell by replacing it with a wild-type allele. As another example, a polynucleotide sequence of a target gene which is responsible for entry or proliferation of a pathogen into a cell may be a suitable target for deletion or insertion to disrupt the function of the target gene to prevent the pathogen from entering the cell or proliferating inside the cell.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, a CRISPR/Cas system includes a Cas protein and at least one to two ribonucleic acids that are capable of directing the Cas protein to and hybridizing to a target motif of a target polynucleotide sequence. As used herein, “protein” and “polypeptide” are used interchangeably to refer to a series of amino acid residues joined by peptide bonds (i.e., a polymer of amino acids) and include modified amino acids (e.g., phosphorylated, glycated, glycosylated, etc.) and amino acid analogs. Exemplary polypeptides or proteins include gene products, naturally occurring proteins, homologs, paralogs, fragments and other equivalents, variants, and analogs of the above.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein. Exemplary Cas core proteins include, but are not limited to Cas1, Cas2, Cas3, Cas4, Cas5, Cas6, Cas7, Cas8, Cas9), and Cas12a. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to, Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (2019): Strecker et al., Science 365, 48-53 (2019).

In some embodiments, a Cas protein comprises any one of the Cas proteins described herein or a functional portion thereof. As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cas12a (also known as Cpf1) protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9) protein comprises a functional portion of the HNH nuclease domain. In some embodiments, a functional portion of the Cas12a protein comprises a functional portion of a RuvC-like domain.

In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In certain embodiments, Cas proteins can be conjugated to or fused to a charged protein (e.g., that carries a positive, negative or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol. 2010; 5(8):747-52). In certain embodiments, the Cas protein can be fused to a protein transduction domain (PTD) to facilitate its entry into a cell. Exemplary PTDs include Tat, oligoarginine, and penetratin. In some embodiments, the Cas9) protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetratin domain. In some embodiments, the Cas9 protein comprises a Cas9 polypeptide fused to a superpositively charged GFP. In some embodiments, the Cas12a protein comprises a Cas12a polypeptide fused to a cell-penetrating peptide. In some embodiments, the Cas12a protein comprises a Cas12a polypeptide fused to a PTD. In some embodiments, the Cas12a protein comprises a Cas12a polypeptide fused to a tat domain. In some embodiments, the Cas12a protein comprises a Cas12a polypeptide fused to an oligoarginine domain. In some embodiments, the Cas12a protein comprises a Cas12a polypeptide fused to a penetratin domain. In some embodiments, the Cas12a protein comprises a Cas12a polypeptide fused to a superpositively charged GFP.

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, viral transduction (e.g., lentiviral transduction) or otherwise delivered on a viral vector (e.g., fusogen-mediated delivery). In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

In some embodiments, the Cas protein is complexed with one to two ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

The methods of the present technology contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs.

In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

Exemplary gRNA sequences useful for CRISPR/Cas-based targeting of genes described herein are provided in Tables 1A-D and Table 15. The sequences of Table 15 can be found in WO2016183041 filed May 9, 2016, the disclosure including the Tables, Appendices, and Sequence Listing is incorporated herein by reference in its entirety.

TABLE 15
Exemplary gRNA sequences useful for targeting genes

| Gene Name | SEQ ID NO: | WO2016183041 |
|---|---|---|
| HLA-A | SEQ ID NOs: 2-1418 | Table 8, Appendix 1 |
| HLA-B | SEQ ID NOs: 1419-3277 | Table 9, Appendix 2 |
| HLA-C | SEQ ID NOs: 3278-5183 | Table 10, Appendix 3 |
| RFX-ANK | SEQ ID NOs: 95636-102318 | Table 11, Appendix 4 |
| NFY-A | SEQ ID NOs: 102319-121796 | Table 13, Appendix 6 |
| RFX5 | SEQ ID NOs: 85645-90115 | Table 16, Appendix 9 |
| RFX-AP | SEQ ID NOs: 90116-95635 | Table 17, Appendix 10 |
| NFY-B | SEQ ID NOs: 121797-135112 | Table 20, Appendix 13 |
| NFY-C | SEQ ID NOs: 135113-176601 | Table 22, Appendix 15 |
| IRF1 | SEQ ID NOs: 176602-182813 | Table 23, Appendix 16 |
| TAP1 | SEQ ID NOs: 182814-188371 | Table 24, Appendix 17 |
| CIITA | SEQ ID NOs: 5184-36352 | Table 12, Appendix 5 |
| B2M | SEQ ID NOs: 81240-85644 | Table 15, Appendix 8 |
| NLRC5 | SEQ ID NOs: 36353-81239 | Table 14, Appendix 7 |
| CD47 | SEQ ID NOs: 200784-231885 | Table 29, Appendix 22 |
| HLA-E | SEQ ID NOs: 189859-193183 | Table 19, Appendix 12 |
| HLA-F | SEQ ID NOs: 688808-699754 | Table 45, Appendix 38 |
| HLA-G | SEQ ID NOs: 188372-189858 | Table 18, Appendix 11 |
| PD-L1 | SEQ ID NOs: 193184-200783 | Table 21, Appendix 14 |

In some embodiments, the cells of the present technology are made using Transcription Activator-Like Effector Nucleases (TALEN) methodologies.

By a “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-I. In a particular embodiment, the TALE domain can be fused to a meganuclease like for instance I-CreI and I-OnuI or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALEN kits are sold commercially.

In some embodiments, the cells are manipulated using zinc finger nuclease (ZFN). A “zinc finger binding protein” is a protein or polypeptide that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues co-ordinated with zinc along with the two cysteine residues of a single beta turn (see, e.g., Berg & Shi, Science 271:1081-1085 (1996)).

In some embodiments, the cells are made using a homing endonuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease may for example correspond to a LAGLIDADG endonuclease, to a HNH endonuclease, or to a GIY-YIG endonuclease. Preferred homing endonuclease can be an I-CreI variant.

In some embodiments, the cells are made using a meganuclease. Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040); Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77: Donoho et al., Mol. Cell. Biol, 1998, 18, 4070-4078; Elliott et al., Mol. Cell. Biol., 1998, 18, 93-101: Cohen-Tannoudji et al., Mol. Cell. Biol., 1998, 18, 1444-1448).

In some embodiments, the cells are made using RNA silencing or RNA interference (RNAi) to knockdown (e.g., decrease, eliminate, or inhibit) the expression of a polypeptide such as a tolerogenic factor. Useful RNAi methods include those that utilize synthetic RNAi molecules, short interfering RNAs (siRNAs), PIWI-interacting NRAs (piRNAs), short hairpin RNAs (shRNAs), microRNAs (miRNAs), and other transient knockdown methods recognized by those skilled in the art. Reagents for RNAi including sequence specific shRNAs, siRNA, miRNAs and the like are commercially available. For instance, CIITA can be knocked down in a pluripotent stem cell by introducing a CIITA siRNA or transducing a CIITA shRNA-expressing virus into the cell. In some embodiments, RNA interference is employed to reduce or inhibit the expression of at least one selected from the group consisting of CIITA, B2M, and NLRC5.

In some embodiments, the cells are made using a CRISPR/Cas system, wherein nucleic acids encoding Cas protein and nucleic acids encoding the at least one to two ribonucleic acids are introduced into a cell via viral transduction (e.g., lentiviral transduction).

In some embodiments, the lentiviral vector comprises one or more fusogens. In some embodiments, the fusogen facilitates the fusion of the lentiviral vector to a membrane. In some embodiments, the membrane is a plasma cell membrane. In some embodiments, the lentiviral vector comprising the fusogen integrates into the membrane into a lipid bilayer of a target cell. In some embodiments, one or more of the fusogens described herein may be included in the lentiviral vector. In some embodiments, the fusogen is a protein fusogen, e.g., a mammalian protein or a homologue of a mammalian protein (e.g., having 50%, 60%, 70%, 80%, 85%, 90%, 95%, 96%, 97%, 98%, 99%, or greater identity), a non-mammalian protein such as a viral protein or a homologue of a viral protein (e.g., having 50%, 60%, 70%, 80%, 85%, 90%, 95%, 96%, 97%, 98%, 99%, or greater identity), a native protein or a derivative of a native protein, a synthetic protein, a fragment thereof, a variant thereof, a protein fusion comprising one or more of the fusogens or fragments, and any combination thereof.

In some embodiments, the fusogen results in mixing between lipids in the lentiviral vector and lipids in the target cell. In some embodiments, the fusogen results in formation of one or more pores between the interior of the viral vector and the cytosol of the target cell.

In some embodiments, the fusogen may include a mammalian protein. Examples of mammalian fusogens may include, but are not limited to, a SNARE family protein such as vSNAREs and tSNAREs, a syncytin protein such as Syncytin-1 (DOI: 10.1128/JVI.76.13.6442-6452.2002), and Syncytin-2, myomaker (biorxiv.org/content/early/2017/04/02/123158, doi.org/10.1101/123158, doi: 10.1096/fj.201600945R, doi: 10.1038/nature12343), myomixer (www.nature.com/nature/journal/v499/n7458/full/nature12343.html, doi: 10.1038/nature12343), myomerger (science.sciencemag.org/content/early/2017/04/05/science.aam9361, DOI: 10.1126/science.aam9361), FGFRL1 (fibroblast growth factor receptor-like 1), Minion (doi.org/10.1101/122697), an isoform of glyceraldehyde-3-phosphate dehydrogenase (GAPDH) (e.g., as disclosed in U.S. Pat. No. 6,099,857A), a gap junction protein such as connexin 43, connexin 40, connexin 45, connexin 32 or connexin 37 (e.g., as disclosed in US 2007/0224176, Hap2, any protein capable of inducing syncytium formation between heterologous cells (see Table 2), any protein with fusogen properties, a homologue thereof, a fragment thereof, a variant thereof, and a protein fusion comprising one or more proteins or fragments thereof. In some embodiments, the fusogen is encoded by a human endogenous retroviral element (hERV) found in the human genome. Additional exemplary fusogens are disclosed in U.S. Pat. No. 6,099,857A and US 2007/0224176, the entire contents of which are hereby incorporated by reference.

In some embodiments, the fusogen may include a non-mammalian protein, e.g., a viral protein. In some embodiments, a viral fusogen is a Class I viral membrane fusion protein, a Class II viral membrane protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some embodiments, Class I viral membrane fusion proteins include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV), and paramyxovirus F proteins.

In some embodiments, Class II viral membrane proteins include, but are not limited to, tick bone encephalitis E (TBEV E), Semliki Forest Virus E1/E2.

In some embodiments, Class III viral membrane fusion proteins include, but are not limited to, rhabdovirus G (e.g., fusogenic protein G of the Vesicular Stomatatis Virus (VSV-G), Cocal virus G protein), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa California multiple NPV (AcMNPV) gp64), and Borna disease virus (BDV) glycoprotein (BDV G).

Examples of other viral fusogens, e.g., membrane glycoproteins and viral fusion proteins, include, but are not limited to: viral syncytia proteins such as influenza hemagglutinin (HA) or mutants, or fusion proteins thereof: human immunodeficiency virus type 1 envelope protein (HIV-1 ENV), gp120 from HIV binding LFA-1 to form lymphocyte syncytium, HIV gp41, HIV gp160, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukaemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of a Morbillivirus (e.g., measles virus (MeV), canine distemper virus, Cetacean morbillivirus, Peste-des-petits-ruminants virus, Phocine distemper virus, Rinderpest virus), Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukaemia virus and Mason Pfizer monkey virus; mumps virus hemagglutinin neuraminidase, and glyoproteins F1 and F2; membrane glycoproteins from Venezuelan equine encephalomyelitis; paramyxovirus F protein; SIV gp160 protein; Ebola virus G protein; or Sendai virus fusion protein, or a homologue thereof, a fragment thereof, a variant thereof, and a protein fusion comprising one or more proteins or fragments thereof.

Non-mammalian fusogens include viral fusogens, homologues thereof, fragments thereof, and fusion proteins comprising one or more proteins or fragments thereof. Viral fusogens include class I fusogens, class II fusogens, class III fusogens, and class IV fusogens. In embodiments, class I fusogens such as human immunodeficiency virus (HIV) gp41, have a characteristic postfusion conformation with a signature trimer of α-helical hairpins with a central coiled-coil structure. Class I viral fusion proteins include proteins having a central postfusion six-helix bundle. Class I viral fusion proteins include influenza HA, parainfluenza F, HIV Env, Ebola GP, hemagglutinins from orthomyxoviruses, F proteins from paramyxoviruses (e.g. Measles, (Katoh et al. BMC Biotechnology 2010, 10:37)), ENV proteins from retroviruses, and fusogens of filoviruses and coronaviruses. In embodiments, class II viral fusogens such as dengue E glycoprotein, have a structural signature of β-sheets forming an elongated ectodomain that refolds to result in a trimer of hairpins. In embodiments, the class II viral fusogen lacks the central coiled coil. Class II viral fusogen can be found in alphaviruses (e.g., E1 protein) and flaviviruses (e.g., E glycoproteins). Class II viral fusogens include fusogens from Semliki Forest virus, Sinbis, rubella virus, and dengue virus. In embodiments, class III viral fusogens such as the vesicular stomatitis virus G glycoprotein, combine structural signatures found in classes I and II. In embodiments, a class III viral fusogen comprises a helices (e.g., forming a six-helix bundle to fold back the protein as with class I viral fusogens), and β sheets with an amphiphilic fusion peptide at its end, reminiscent of class II viral fusogens. Class III viral fusogens can be found in rhabdoviruses and herpesviruses. In embodiments, class IV viral fusogens are fusion-associated small transmembrane (FAST) proteins (doi: 10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In embodiments, the class IV viral fusogens are sufficiently small that they do not form hairpins (doi: 10.1146/annurev-cellbio-101512-122422, doi: 10.1016/j.devcel.2007.12.008).

In some embodiments, lentiviral vectors disclosed herein include one or more CD8 binding agents. For example, a CD8 binding agent may be fused to or incorporated in a protein fusogen or viral envelope protein. In another embodiment, a CD8 binding agent may be incorporated into the viral envelope via fusion with a transmembrane domain.

Exemplary CD8 binding agents include antibodies and fragments thereof (e.g., scFv, VHH) that bind to one or more of CD8 alpha and CD8 beta. Such antibodies may be derived from any species, and may be for example, mouse, rabbit, human, humanized, or camelid antibodies. Exemplary antibodies include those disclosed in WO2014025828, WO2014164553, WO2020069433, WO2015184203, US20160176969, WO2017134306, WO2019032661, WO2020257412, WO2018170096, WO2020060924, U.S. Ser. No. 10/730,944, US20200172620, and the non-human antibodies OKT8; RPA-T8, 12.C7 (Novus); 17D8, 3B5, LT8, RIV11, SP16, YTC182.20, MEM-31, MEM-87, RAVB3, C8/144B (Thermo Fisher); 2ST8.5H7, Bu88, 3C39, Hit8a, SPM548, CA-8, SK1, RPA-T8 (GeneTex); UCHT4 (Absolute Antibody); BW135/80 (Miltenyi); G42-8 (BD Biosciences); C8/1779R, mAB 104 (Enzo Life Sciences); B-Z31 (Sapphire North America); 32-M4, 5F10, MCD8, UCH-T4, 5F2 (Santa Cruz); D8A8Y, RPA-T8 (Cell Signaling Technology). Other exemplary binding agents include designed ankyrin repeat proteins (DARPins) and binding agents based on fibronectin type III (Fn3) scaffolds.

In some embodiments, lentiviral vectors disclosed herein include one or more CD4 binding agents. For example, a CD4 binding agent may be fused to or incorporated in a protein fusogen or viral envelope protein. In another embodiment, a CD4 binding agent may be incorporated into the viral envelope via fusion with a transmembrane domain. Any CD4 binding agent known to those skilled in the art in view of the present disclosure can be used.

In some embodiments, exogenous polynucleotides, e.g., polynucleotides expressing CD47, polynucleotides expressing one or more CARs, and/or polynucleotides encoding Cas protein and nucleic acids encoding at least one to two ribonucleic acids are introduced into a cell via fusogen-mediated delivery. In some embodiments, the fusogen-mediated delivery is carried out in vivo in the recipient patient. In some embodiments, the fusogen-mediated delivery comprises contacting the recipient patient with a composition comprising lentiviral vectors comprising (i) a CD8 binding agent. (ii) polynucleotides encoding CRISPR/Cas gene editing components, and (iii) a polynucleotide encoding CD47, wherein a hypoimmunogenic T cell, non-activated T cell, or population of hypoimmunogenic T cells of the recipient patient is transduced with the lentiviral vectors. In some embodiments, the fusogen-mediated delivery comprises contacting the recipient patient with a composition comprising lentiviral vectors comprising (i) a CD8 binding agent. (ii) polynucleotides encoding CRISPR/Cas gene editing components, and (iii) one or more polynucleotides encoding the one or more CARs, wherein a hypoimmunogenic T cell, non-activated T cell, or population of hypoimmunogenic T cells of the recipient patient is transduced with the lentiviral vectors. In some embodiments, the fusogen-mediated delivery comprises contacting the recipient patient with a composition comprising lentiviral vectors comprising (i) a CD8 binding agent, and (ii) polynucleotides encoding CRISPR/Cas gene editing components targeting the RHD locus, wherein a hypoimmunogenic T cell, non-activated T cell, or population of hypoimmunogenic T cells of the recipient patient is transduced with the lentiviral vectors. In some embodiments, the fusogen-mediated delivery comprises contacting the recipient patient with a composition comprising lentiviral vectors comprising (i) a CD8 binding agent, (ii) polynucleotides encoding CRISPR/Cas gene editing components targeting the RHD locus, and (iii) one or more polynucleotides encoding the one or more CARs wherein a hypoimmunogenic T cell, non-activated T cell, or population of hypoimmunogenic T cells of the recipient patient is transduced with the lentiviral vectors. In some embodiments, the one or more polynucleotides encoding the one or more CARs are inserted into the CRISPR/Cas-targeted RHD locus.

### M. Methods for Administering Hypoimmunogenic T Cells

As is described in further detail herein, provided herein are methods for treating a patient who has received an allogeneic transplant or a patient who is or has been pregnant (e.g., having or having had alloimmunization in pregnancy), or who is sensitized against alloantigens, such as a patient who has received an allogeneic transplant or a patient who is or has been pregnant. In some embodiments, the allogeneic transplant includes, but not limited to, an allogeneic cell transplant, an allogeneic blood transfusion, an allogeneic tissue transplant, or an allogeneic organ transplant. In some embodiments, the patient is sensitized against RhD antigen. Examples of patients sensitized against RhD antigen include, e.g., an RhD negative mother with an RhD positive fetus, and an RhD negative recipient patient of an RhD positive cell therapy.

The methods of treating such a patient are generally through administrations of cells, particularly hypoimmunogenic T cells. As will be appreciated, for all the multiple embodiments described herein related to the cells and/or the timing of therapies, the administering of the cells is accomplished by a method or route that results in at least partial localization of the introduced cells at a desired site. The cells can be implanted directly to the desired site, or alternatively be administered by any appropriate route which results in delivery to a desired location in the subject where at least a portion of the implanted cells or components of the cells remain viable. In some embodiments, the cells are administered to treat a disease or disorder, such as any disease, disorder, condition, or symptom thereof that can be alleviated by cell therapy.

In some embodiments, the population of cells is administered at least 1 week (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) or more after the patient is sensitized or exhibits characteristics or features of sensitization. In some embodiments, the population of cells is administered at least 1 month (e.g., 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, or more) or more after the patient has received the allogeneic transplant, has been pregnant (e.g., having or having had alloimmunization in pregnancy) or is sensitized or exhibits characteristics or features of sensitization.

In some embodiments, the administered population of hypoimmunogenic T cells elicits a decreased or lower level of immune activation in the patient. In some instances, the level of immune activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation produced by the administration of immunogenic cells. In some embodiments, the administered population of hypoimmunogenic T cells fails to elicit immune activation in the patient.

In some embodiments, the administered population of hypoimmunogenic T cells elicits a decreased or lower level of systemic TH1 activation in the patient. In some instances, the level of systemic TH1 activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of systemic TH1 activation produced by the administration of immunogenic cells. In some embodiments, the administered population of hypoimmunogenic T cells fails to elicit systemic TH1 activation in the patient.

In some embodiments, the administered population of hypoimmunogenic T cells elicits a decreased or lower level of immune activation of peripheral blood mononuclear cells (PBMCs) in the patient. In some instances, the level of immune activation of PBMCs elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation of PBMCs produced by the administration of immunogenic cells. In some embodiments, the administered population of hypoimmunogenic T cells fails to elicit immune activation of PBMCs in the patient.

In some embodiments, the administered population of hypoimmunogenic T cells elicits a decreased or lower level of donor-specific IgG antibodies in the patient. In some instances, the level of donor-specific IgG antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgG antibodies produced by the administration of immunogenic cells. In some embodiments, the administered population of hypoimmunogenic T cells fails to elicit donor-specific IgG antibodies in the patient.

In some embodiments, the administered population of hypoimmunogenic T cells elicits a decreased or lower level of IgM and IgG antibody production in the patient. In some instances, the level of IgM and IgG antibody production elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of IgM and IgG antibody production produced by the administration of immunogenic cells. In some embodiments, the administered population of hypoimmunogenic T cells fails to elicit IgM and IgG antibody production in the patient.

In some embodiments, the administered population of hypoimmunogenic T cells elicits a decreased or lower level of cytotoxic T cell killing in the patient. In some instances, the level of cytotoxic T cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of cytotoxic T cell killing produced by the administration of immunogenic cells. In some embodiments, the administered population of hypoimmunogenic T cells fails to elicit cytotoxic T cell killing in the patient.

As discussed above, provided herein are cells that in certain embodiments can be administered to a patient sensitized against alloantigens such as RhD and/or human leukocyte antigens. In some embodiments, the patient is or has been pregnant, e.g., with alloimmunization in pregnancy (e.g., hemolytic disease of the fetus and new born (HDFN), neonatal alloimmune neutropenia (NAN) or fetal and neonatal alloimmune thrombocytopenia (FNAIT)). In other words, the patient has or has had a disorder or condition associated with alloimmunization in pregnancy such as, but not limited to, hemolytic disease of the fetus and newborn (HDFN), neonatal alloimmune neutropenia (NAN), and fetal and neonatal alloimmune thrombocytopenia (FNAIT). In some embodiments, the patient has received an allogeneic transplant such as, but not limited to, an allogeneic cell transplant, an allogeneic blood transfusion, an allogeneic tissue transplant, or an allogeneic organ transplant. In some embodiments, the patient exhibits memory B cells against alloantigens. In some embodiments, the patient exhibits memory T cells against alloantigens. Such patients can exhibit both memory B and memory T cells against alloantigens.

Upon administration of the cells described, the patient exhibits no systemic immune response, or a reduced level of systemic immune response compared to responses to cells that are not hypoimmunogenic. In some embodiments, the patient exhibits no adaptive immune response, or a reduced level of adaptive immune response compared to responses to cells that are not hypoimmunogenic. In some embodiments, the patient exhibits no innate immune response, or a reduced level of innate immune response compared to responses to cells that are not hypoimmunogenic. In some embodiments, the patient exhibits no T cell response, or a reduced level of T cell response compared to responses to cells that are not hypoimmunogenic. In some embodiments, the patient exhibits no B cell response, or a reduced level of B cell response compared to responses to cells that are not hypoimmunogenic.

As is described in further detail herein, provided herein is a population of hypoimmunogenic T cells including exogenous CD47 polypeptides and reduced expression of RhD antigen and MHC class I human leukocyte antigens, a population of hypoimmunogenic T cells including exogenous CD47 polypeptides and reduced expression of RhD antigen and MHC class II human leukocyte antigens, and a population of hypoimmunogenic T cells including exogenous CD47 polypeptides and reduced expression of RhD antigen and MHC class I and class II human leukocyte antigens.

Provided herein are methods for treating a patient with a condition, disorder, or disorder includes administration of a population of hypoimmunogenic T cells (e.g., hypoimmunogenic T cells and non-activated T cells propagated from primary T cells or progeny thereof, or hypoimmunogenic T cells and non-activated T cells derived from an induced pluripotent stem cell (iPSC) or a progeny thereof) to a subject, e.g., a human patient. For instance, a population of hypoimmunogenic primary T cells such as, but not limited to, CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells that express CD45RA (TEMRA cells), tissue-resident memory (Trm) cells, virtual memory T cells, innate memory T cells, memory stem cell (Tsc), γδ T cells, and any other subtype of T cell is administered to a patient to treat a condition, disorder, or disorder. In some embodiments, an immunosuppressive and/or immunomodulatory agent (such as, but not limited to a lymphodepletion agent) is not administered to the patient before the administration of the population of hypoimmunogenic T cells. In some embodiments, an immunosuppressive and/or immunomodulatory agent is administered at least 1, 2, 3, 4, 5, 6, 7, 8, 9, 10, 11, 12, 13, 14 days or more before the administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the administration of the cells. In numerous embodiments, an immunosuppressive and/or immunomodulatory agent is not administered to the patient after the administration of the cells, or is administered at least 1, 2, 3, 4, 5, 6, 7, 8, 9, 10, 11, 12, 13, 14 days or more after the administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the administration of the cells. In some embodiments where an immunosuppressive and/or immunomodulatory agent is administered to the patient before or after the administration of the cells, the administration is at a lower dosage than would be required for cells with RhD antigen, MHC I and/or MHC II expression and without exogenous expression of CD47.

Non-limiting examples of an immunosuppressive and/or immunomodulatory agent (such as, but not limited to a lymphodepletion agent) include cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-α and similar agents. In some embodiments, the immunosuppressive and/or immunomodulatory agent is selected from a group of immunosuppressive antibodies consisting of antibodies binding to p75 of the IL-2 receptor, antibodies binding to, for instance, MHC, CD2, CD3, CD4, CD7, CD28, B7, CD40), CD45, IFN-gamma, TNF-alpha, IL-4, IL-5, IL-6R, IL-6, IGF, IGFR1, IL-7, IL-8, IL-10, CD11a, or CD58, and antibodies binding to any of their ligands. In some embodiments, such an immunosuppressive and/or immunomodulatory agent may be selected from soluble IL-15R, IL-10, B7 molecules (e.g., B7-1, B7-2, variants thereof, and fragments thereof), ICOS, and OX40, an inhibitor of a negative T cell regulator (such as an antibody against CTLA-4) and similar agents.

In some embodiments, where an immunosuppressive and/or immunomodulatory agent is administered to the patient before or after the administration of the cells, the administration is at a lower dosage than would be required for cells with RhD antigen expression, MHC I and/or MHC II expression, TCR expression and without exogenous expression of CD47. In some embodiments, where an immunosuppressive and/or immunomodulatory agent is administered to the patient before or after the first administration of the cells, the administration is at a lower dosage than would be required for cells with RhD antigen expression, MHC I and MHC II expression, TCR expression and without exogenous expression of CD47.

For therapeutic application, cells prepared according to the disclosed methods can typically be supplied in the form of a pharmaceutical composition comprising an isotonic excipient, and are prepared under conditions that are sufficiently sterile for human administration. For general principles in medicinal formulation of cell compositions, see “Cell Therapy: Stem Cell Transplantation, Gene Therapy, and Cellular Immunotherapy,” by Morstyn & Sheridan eds, Cambridge University Press, 1996; and “Hematopoietic Stem Cell Therapy,” E. D. Ball, J. Lister & P. Law, Churchill Livingstone, 2000. The cells can be packaged in a device or container suitable for distribution or clinical use.

### N. Generation of Hypoimmunogenic Pluripotent Stem Cells

The present technology provides methods of producing hypoimmunogenic T cells and non-activated T cells derived from pluripotent cells. In some embodiments, the method comprises generating pluripotent stem cells. The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPCSs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al., World J. Stem Cells 7(1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogenous genes.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g., fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT: SOX2, OCT4 (POU5F1), KLF4, MYC, NANOG, LIN28, and SV40L T antigen. In general, these reprogramming factor genes are provided on episomal vectors such as are known in the art and commercially available.

In general, as is known in the art, iPSCs are made from non-pluripotent cells such as, but not limited to, blood cells, fibroblasts, etc., by transiently expressing the reprogramming factors as described herein.

### O. Assays for Hypoimmunogenicity Phenotypes

Once the hypoimmunogenic T cells have been generated, they may be assayed for their hypoimmunogenicity as is described in WO2016183041 and WO2018132783.

In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g. teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell responses can be assessed by Elispot, ELISA, FACS, PCR, or mass cytometry (CYTOF). B cell responses or antibody responses are assessed using FACS or Luminex. Additionally, or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassay's such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of hypoimmunogenic T cells is determined using an allogenic humanized immunodeficient mouse model. In some instances, the hypoimmunogenic T cells are transplanted into an allogenic humanized NSG-SGM3 mouse and assayed for cell rejection, cell survival, and teratoma formation. In some instances, grafted hypoimmunogenic T cells or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

As will be appreciated by those in the art, the successful reduction of the RhD antigen levels in the cells can be measured using techniques known in the art and as described below; for example, Western blotting and FACS techniques using labeled antibodies that bind the RhD antigen, for example, using commercially available RhD antibodies, RT-PCR techniques, etc.

In addition, the cells can be tested to confirm that the RhD antigen is not expressed on the cell surface. Again, this assay is done as is known in the art and generally is done using either Western Blots or FACS analysis based on commercial antibodies that bind to human RhD antigen.

The successful reduction of MHC I function (HLA I when the cells are derived from human cells) in the pluripotent cells can be measured using techniques known in the art and as described below; for example, FACS techniques using labeled antibodies that bind the HLA complex; for example, using commercially available HLA-A, B, C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the MHC II function (HLA II when the cells are derived from human cells) in the pluripotent cells or their derivatives can be measured using techniques known in the art such as Western blotting using antibodies to the protein, FACS techniques, RT-PCR techniques, etc.

In addition, the cells can be tested to confirm that the HLA II complex is not expressed on the cell surface. Again, this assay is done as is known in the art (See FIG. 21 of WO2018132783, for example) and generally is done using either Western Blots or FACS analysis based on commercial antibodies that bind to human HLA Class II HLA-DR. DP and most DQ antigens.

In addition to the reduction of RhD, HLA I and II (or MHC I and II), the hypoimmunogenic T cells and non-activated T cells of the technology have a reduced susceptibility to macrophage phagocytosis and NK cell killing. The resulting hypoimmunogenic T cells “escape” the immune macrophage and innate pathways. The cells can be tested to confirm reduced complement-dependent cytotoxicity (CDC) and antibody-dependent cellular cytotoxicity (ADCC) using standard techniques known in the art, such as those described below.

P. Administration of Hypoimmunogenic T Cells Differentiated from Hypoimmunogenic Pluripotent Cells

The present technology provides HIP cells that are differentiated into different cell types for subsequent transplantation into recipient subjects. Differentiation can be assayed as is known in the art, generally by evaluating the presence of cell-specific markers. As will be appreciated by those in the art, the differentiated hypoimmunogenic pluripotent cell derivatives can be transplanted using techniques known in the art that depends on both the cell type and the ultimate use of these cells. In some embodiments, T lymphocytes (T cells) are derived from the hypoimmunogenic induced pluripotent stem (HIP) cells described herein. In some embodiments, the T cells derived from HIP cells are administered as a mixture of CD4+ and CD8+ cells. In some embodiments, the T cells derived from HIP cells that are administered are CD4+ cells. In some embodiments the T cells derived from HIP cells that are administered are CD8+ cells. In some embodiments, the T cells derived from HIP cells are administered as non-activated T cells.

Provided herein, T lymphocytes (T cells) are derived from the hypoimmunogenic induced pluripotent stem (HIP) cells described. Methods for generating T cells, including CAR T cells, from pluripotent stem cells (e.g., iPSCs) are described, for example, in Iriguchi et al., Nature Communications 12, 430 (2021); Themeli et al., Cell Stem Cell, 16(4):357-366 (2015); Themeli et al., Nature Biotechnology 31:928-933 (2013).

In some embodiments, the hypoimmunogenic induced pluripotent stem cell-derived T cell includes one or more chimeric antigen receptors (CARs). Any suitable CAR can be included in the hypoimmunogenic induced pluripotent stem cell-derived T cell, including the CARs described herein. In some embodiments, the hypoimmunogenic induced pluripotent stem cell-derived T cell includes one or more polynucleotides encoding one or more CARs. Any suitable method can be used to insert the one or more CARs into a genomic locus of the hypoimmunogenic T cell including the gene editing methods described herein (e.g., a CRISPR/Cas system).

HIP-derived T cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

### IV. Examples

### Example 1: RhD Expression on T Cells

To determine whether RhD antigen was expressed on T cells, T cells from five RhD+ human donors were sorted for CD3 expression to generate a CD3+ population, and the CD3+ T cells were analyzed for RhD antigen expression using standard techniques. The T cells were analyzed by flow cytometry (using standard methods) after thawing or after activation with IL-2. CD3+ T cells from two RhD− donors served as a control.

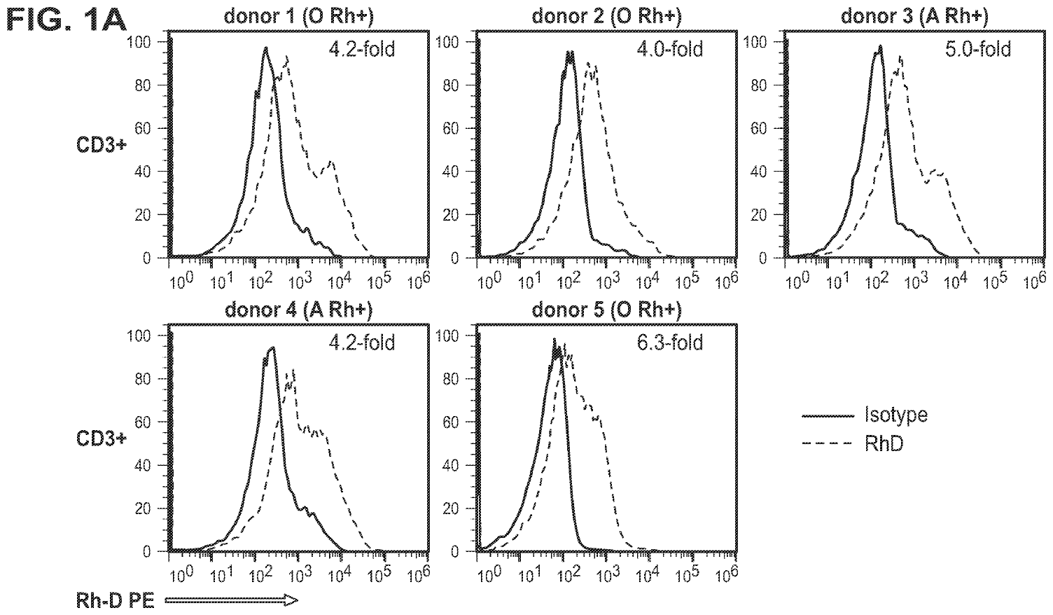

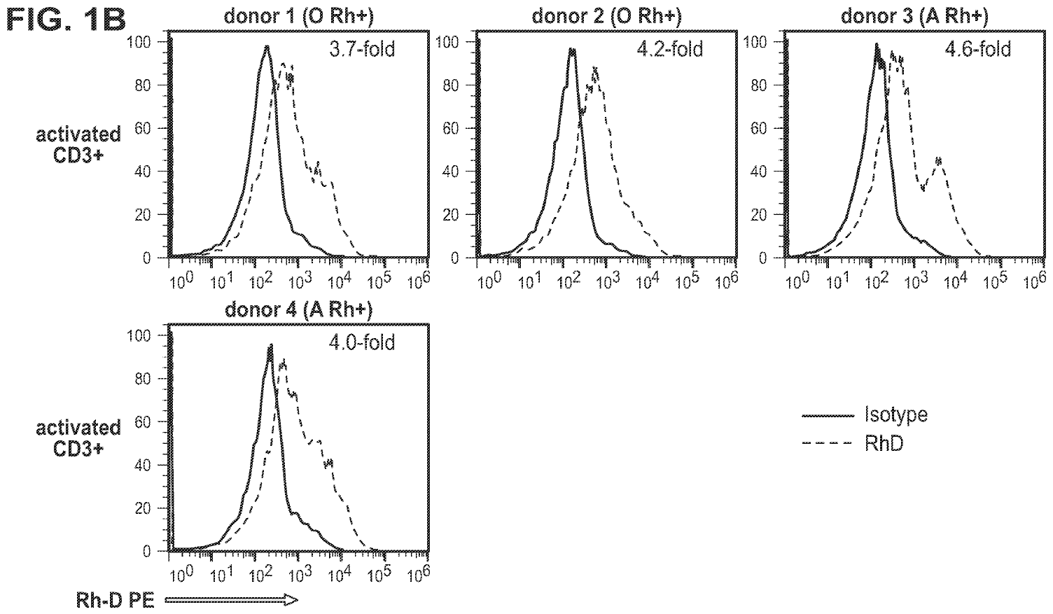

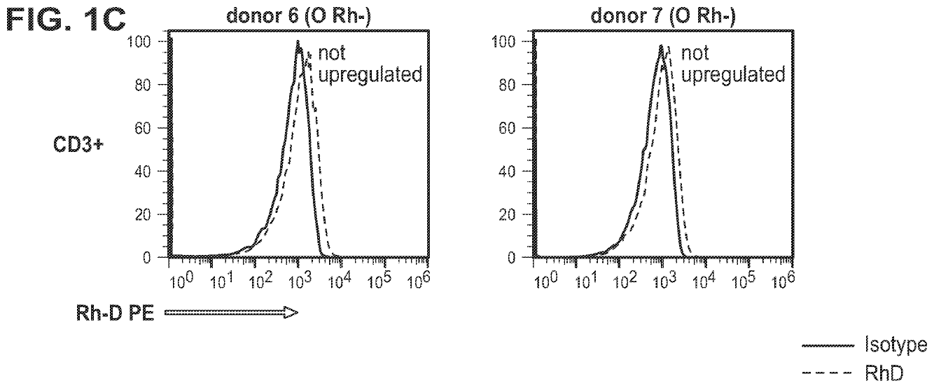

Cells were blocked with anti-Fc receptor antibodies and stained with an anti-CD3 antibody as well as an anti-RhD antibody (CD240D) that was concentration matched to an isotype control. As shown in FIGS. 1A and 1B, RhD antigen was expressed on T cells from RhD+ donors, and expression was not affected following activation with IL-2. RhD antigen was not expressed on T cells from RhD− donors before or after activation with IL-2 (FIG. 1C).

In view of the surprising finding that RhD antigen is expressed on T cells including activated T cells, the functional relevance of its expression was analyzed.

### ADCC (Antibody-Dependent Cellular Cytotoxicity)

The Xcelligence cell killing assay was used to determine whether macrophages or natural killer (NK) cells recognize and kill RhD+ T cells in the presence of Roledumab, a monoclonal IgG1-type antibody that binds to RhD.

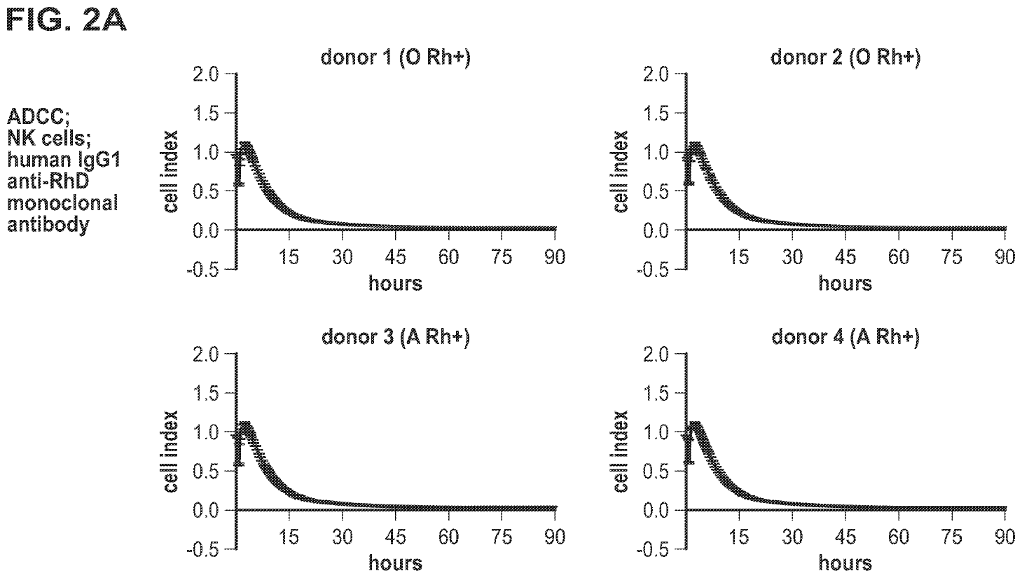

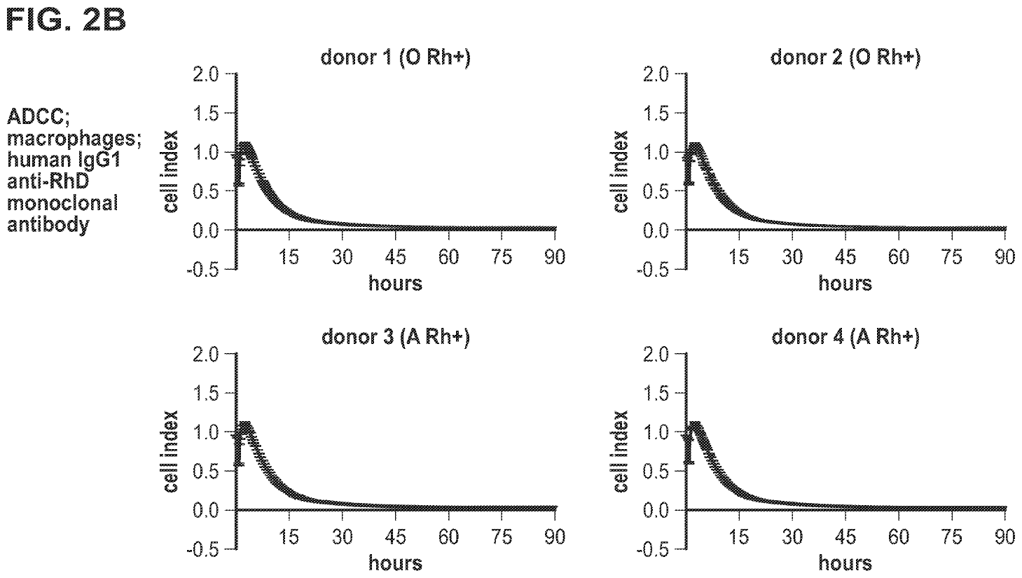

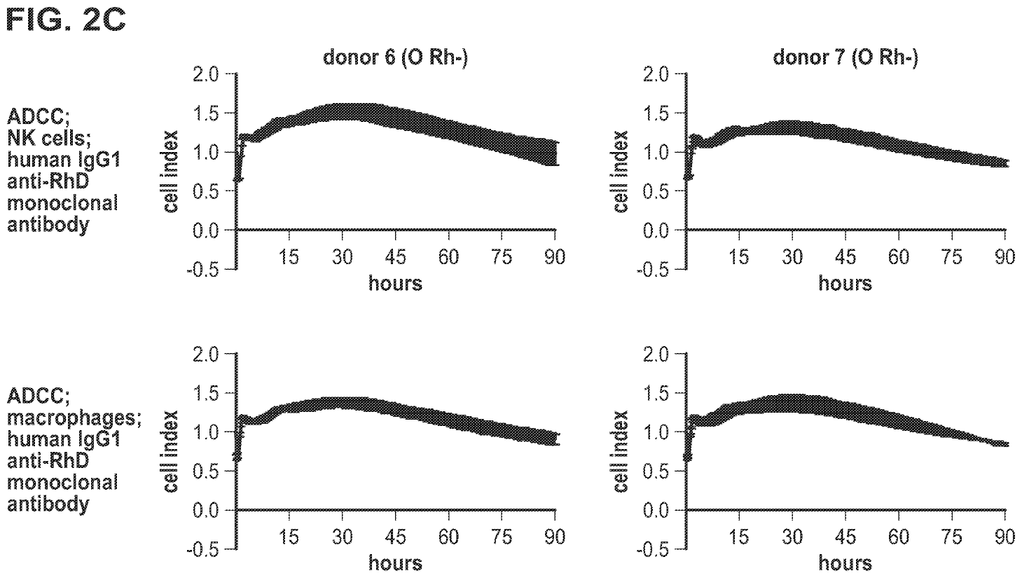

As shown in FIGS. 2A-2C, RhD+ T cells were killed by NK cells (FIG. 2A) or macrophages (FIG. 2B) by ADCC in the presence of Roledumab, and there was no killing of the RhD− T cells in the presence of anti-RhD antibodies (FIG. 2C).

### CDC (Complement-Dependent Cytotoxicity)

The Xcelligence cell killing assay was used to determine whether CDC would be triggered by RhD+ T cells in the presence of Roledumab.

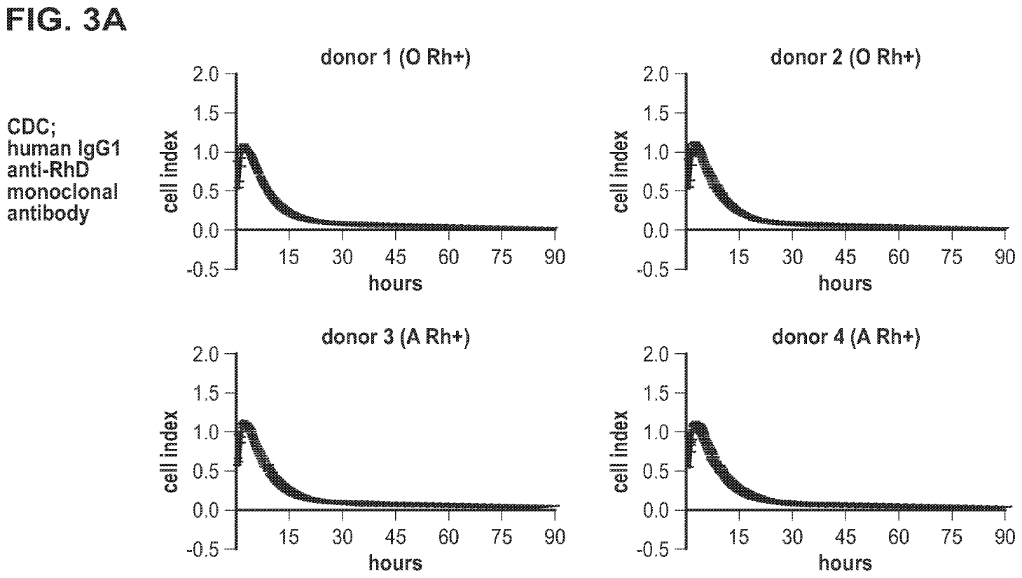

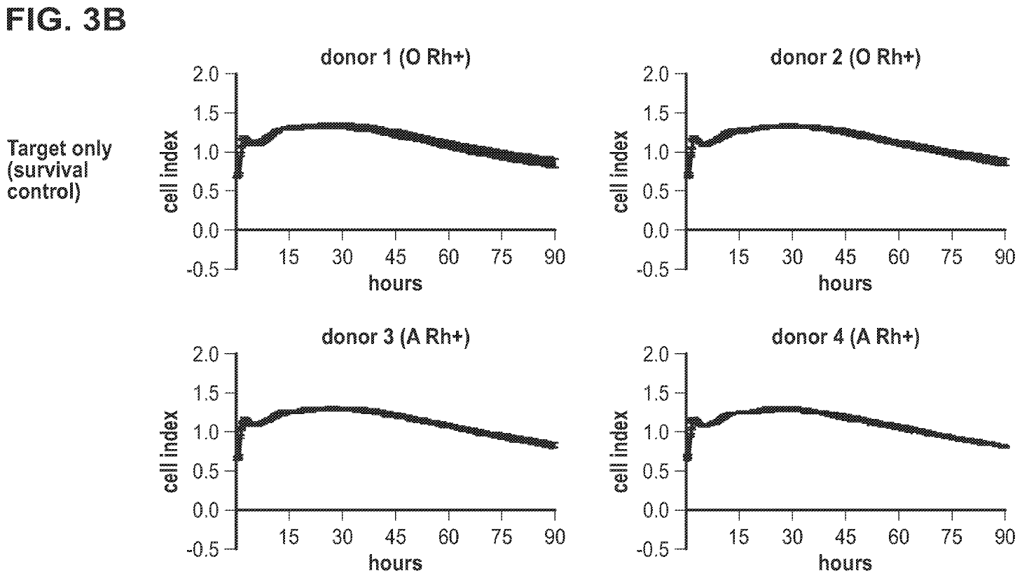

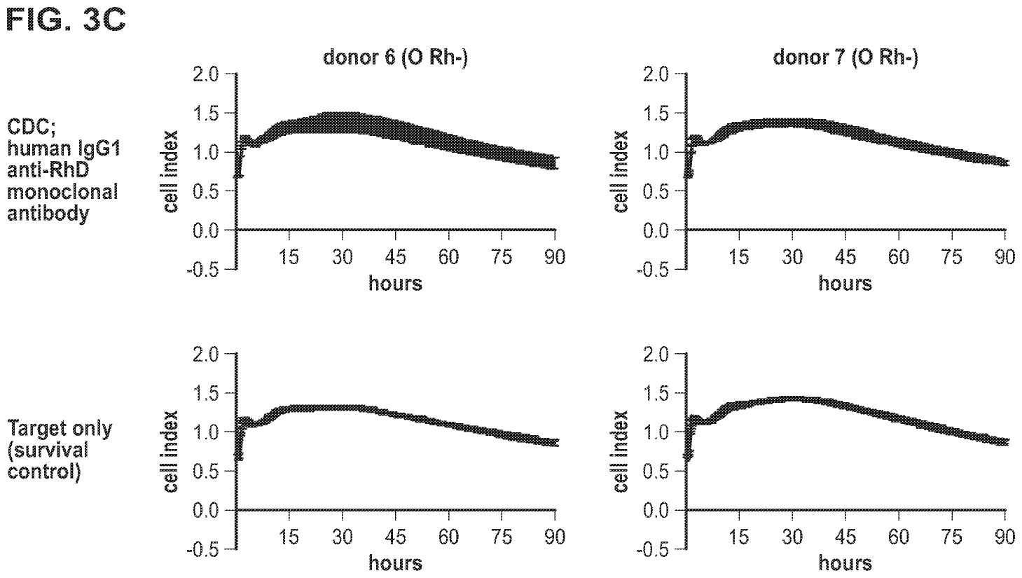

As shown in FIGS. 3A-3C, RhD+ T cells were killed by CDC in the presence of Roledumab, and there was no killing of the RhD− T cells in the presence of anti-RhD antibodies.

### Example 2: RhD Sensitized Patients

T cells were prepared from RhD+ and RhD− donors as in Example 1. ADCC and CDC assays were carried out using serum from RhD+, RhD−, and RhD− sensitized volunteers as in Example 1 to analyze the effect of RhD sensitization on RhD negative recipients.

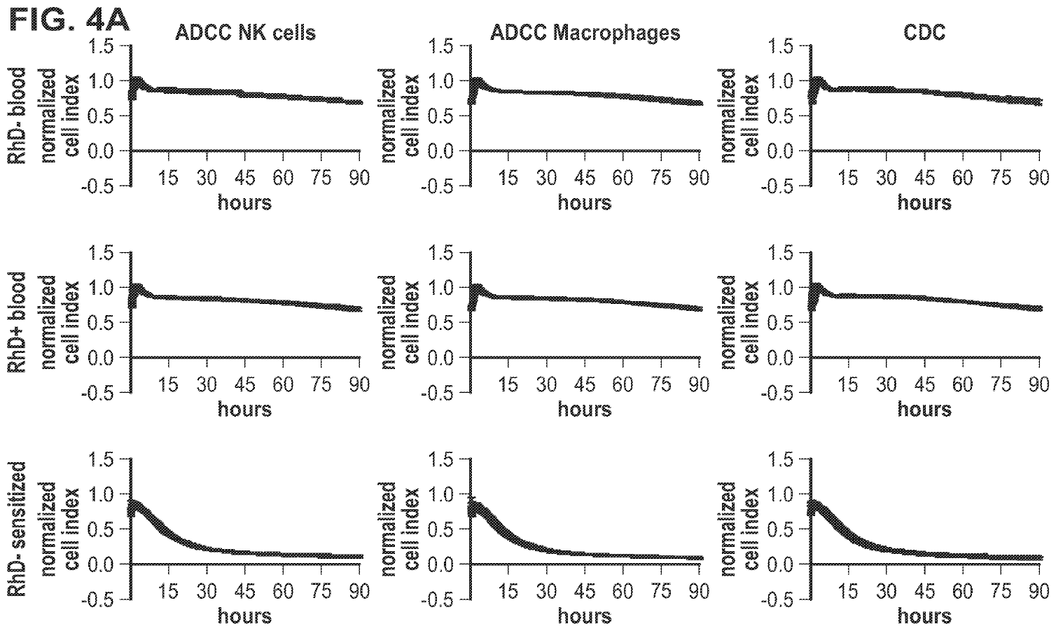

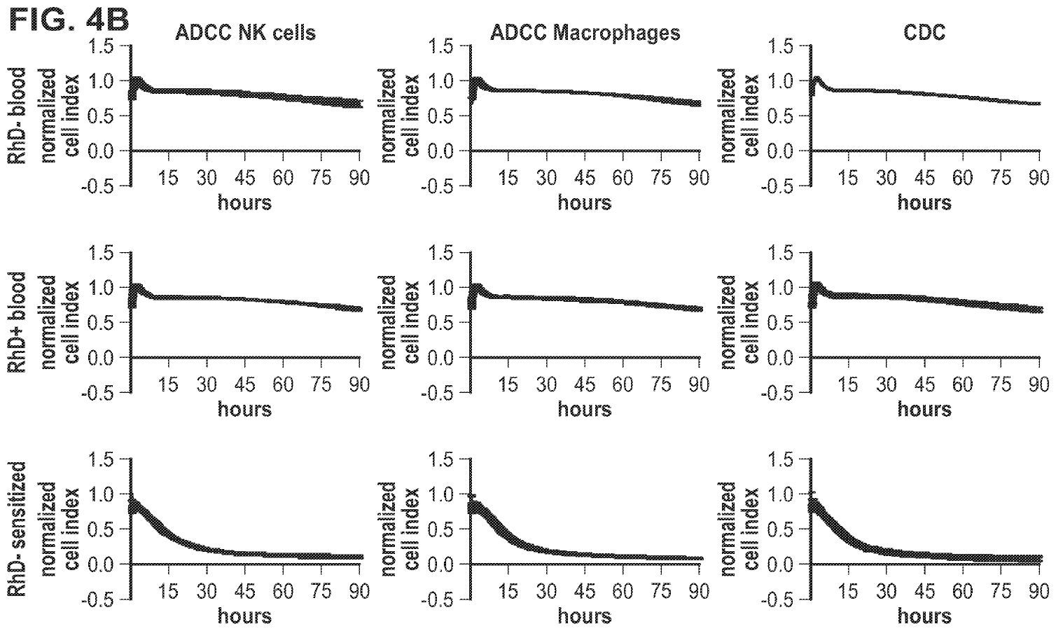

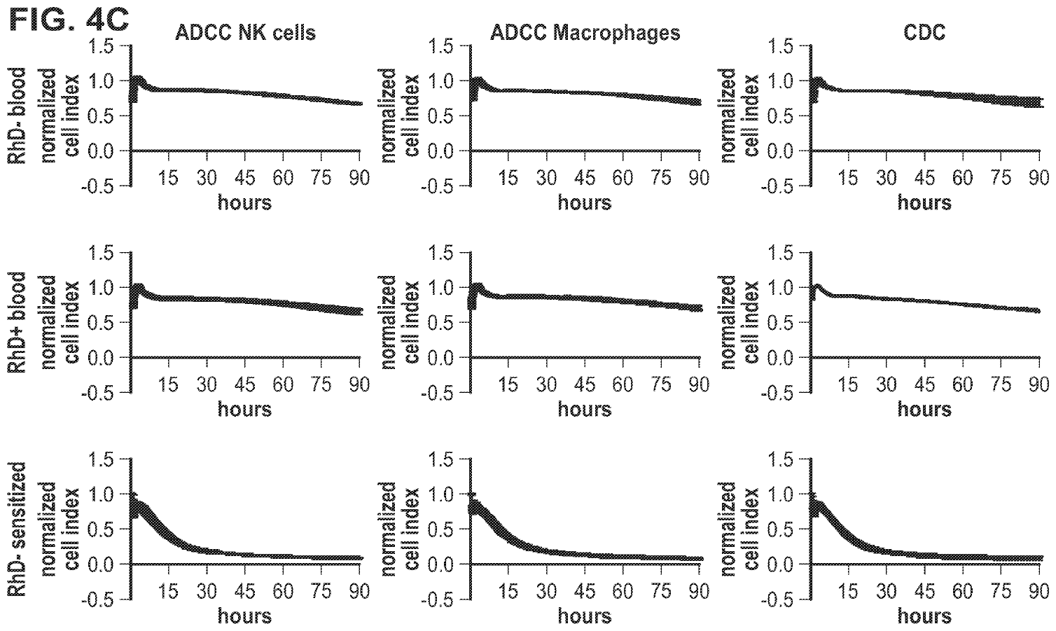

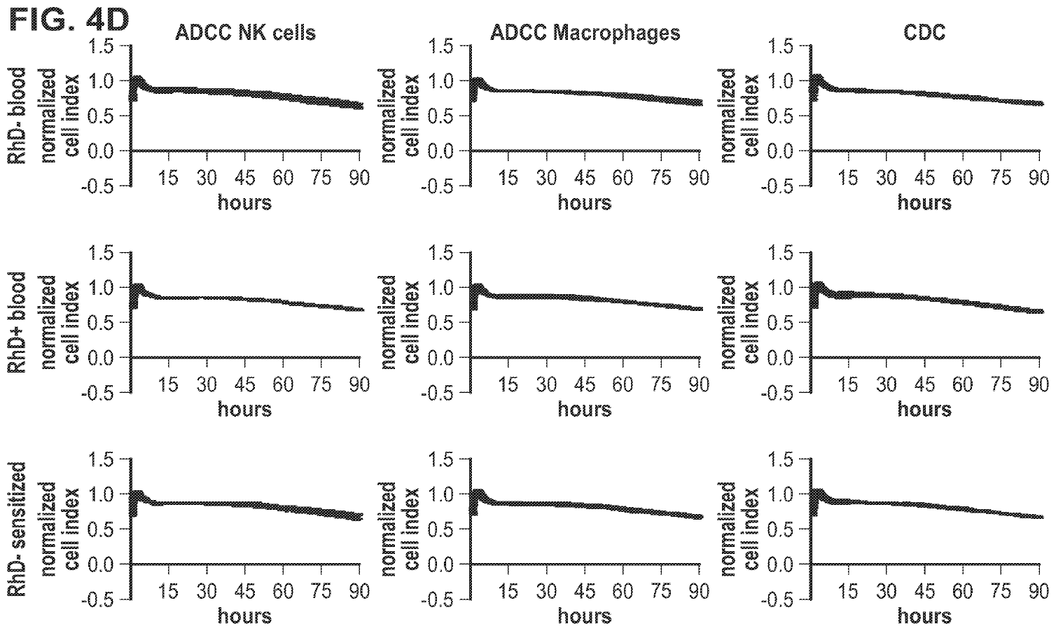

The effect of RhD sensitization on RhD negative recipients was then analyzed. Serum from RhD negative volunteers who were sensitized against RhD was analyzed for killing by CDC and ADCC of RhD+ T cells (blood type O). As shown in FIGS. 4A-C, there was no killing of RhD+ T cells by RhD positive or negative serum, but there was killing of RhD+ T cells when the RhD negative volunteer was previously sensitized. Serum from RhD negative volunteers who were not sensitized was used as control. As shown in FIG. 4D, in the case of the control, there was no killing by RhD positive or negative serum, even in the case of an RhD negative volunteer who was previously sensitized, when the donor cell was RhD negative.

All headings and section designations are used for clarity and reference purposes only and are not to be considered limiting in any way. For example, those of skill in the art will appreciate the usefulness of combining various aspects from different headings and sections as appropriate according to the spirit and scope of the present technology described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. A hypoimmunogenic T cell comprising reduced expression of Rhesus factor D (RhD) antigen and major histocompatibility complex (MHC) class I and/or class II human leukocyte antigens relative to an unaltered or unmodified wild-type cell, and a first exogenous polynucleotide encoding CD47, wherein the hypoimmunogenic T cell is propagated from a primary T cell or a progeny thereof, or is derived from an induced pluripotent stem cell (iPSC) or a progeny thereof.

2. The hypoimmunogenic T cell of claim 1, wherein the hypoimmunogenic T cell is propagated from a primary T cell or a progeny thereof, wherein the primary T cell or progeny thereof comprises reduced expression of RhD antigen and MHC class I and/or class II human leukocyte antigens relative to an unaltered or unmodified wild-type cell, and a first exogenous polynucleotide encoding CD47.

3. The hypoimmunogenic T cell of claim 1, wherein the hypoimmunogenic T cell is derived from an iPSC or a progeny thereof, wherein the iPSC or progeny thereof comprises reduced expression of RhD antigen and MHC class I and/or class II human leukocyte antigens relative to an unaltered or unmodified wild-type cell, and a first exogenous polynucleotide encoding CD47.

4. A non-activated T cell comprising reduced expression of RhD antigen and MHC class I and/or class II human leukocyte antigens relative to an unaltered or unmodified wild-type cell, and a first exogenous polynucleotide encoding CD47, wherein the non-activated T cell is propagated from a primary T cell or a progeny thereof, or is derived from an iPSC or a progeny thereof.

5. The non-activated T cell of claim 4, wherein the non-activated T cell is propagated from a primary T cell or a progeny thereof, wherein the primary T cell or progeny thereof comprises reduced expression of RhD antigen and MHC class I and/or class II human leukocyte antigens relative to an unaltered or unmodified wild-type cell, and a first exogenous polynucleotide encoding CD47.

6. The non-activated T cell of claim 4, wherein the non-activated T cell is derived from an iPSC or a progeny thereof, wherein the iPSC or progeny thereof comprises reduced expression of RhD antigen and MHC class I and/or class II human leukocyte antigens relative to an unaltered or unmodified wild-type cell, and a first exogenous polynucleotide encoding CD47.

7. The non-activated T cell of any one of claims 4-6, wherein the non-activated T cell is a non-activated hypoimmunogenic cell.

8. A population of hypoimmunogenic T cells comprising reduced expression of RhD antigen and MHC class I and/or class II human leukocyte antigens relative to an unaltered or unmodified wild-type cell, and a first exogenous polynucleotide encoding CD47, wherein the population of hypoimmunogenic T cells is propagated from primary T cells or progeny thereof, or is derived from an iPSC or a progeny thereof.

9. The population of hypoimmunogenic T cells of claim 8, wherein the population of hypoimmunogenic T cells is propagated from a primary T cell or a progeny thereof, wherein the primary T cell or progeny thereof comprises reduced expression of RhD antigen and MHC class I and/or class II human leukocyte antigens relative to an unaltered or unmodified wild-type cell, and a first exogenous polynucleotide encoding CD47.

10. The population of hypoimmunogenic T cells of claim 8, wherein the population of hypoimmunogenic T cells is derived from an iPSC or a progeny thereof, wherein the iPSC or progeny thereof comprises reduced expression of RhD antigen and MHC class I and/or class II human leukocyte antigens relative to an unaltered or unmodified wild-type cell, and a first exogenous polynucleotide encoding CD47.

11. The hypoimmunogenic T cell, non-activated T cell, or population of hypoimmunogenic T cells of any one of claims 3-10, wherein the hypoimmunogenic T cell, non-activated T cell, or population of hypoimmunogenic T cells does not express MHC class I and/or class II human leukocyte antigens.

12. The hypoimmunogenic T cell, non-activated T cell, or population of hypoimmunogenic T cells of any one of claims 1-11, wherein the hypoimmunogenic T cell, non-activated T cell, or population of hypoimmunogenic T cells comprises reduced expression of beta-2-microglobulin (B2M) and/or MHC class II transactivator (CIITA) relative to an unaltered or unmodified wild-type cell.

13. The hypoimmunogenic T cell, non-activated T cell, or population of hypoimmunogenic T cells of claim 12, wherein the hypoimmunogenic T cell, non-activated T cell, or population of hypoimmunogenic T cells does not express B2M and/or CIITA.

14. The hypoimmunogenic T cell, non-activated T cell, or population of hypoimmunogenic T cells of any one of claims 1-13, wherein reduced expression of RhD antigen is caused by a knock out of the RHD gene.

15. The hypoimmunogenic T cell, non-activated T cell, or population of hypoimmunogenic T cells of any one of claims 1-14, wherein the hypoimmunogenic T cell, non-activated T cell, or population of hypoimmunogenic T cells does not express RhD antigen.

16. The hypoimmunogenic T cell, non-activated T cell, or population of hypoimmunogenic T cells of any one of claims 1-15, further comprising reduced expression of a T cell receptor relative to an unaltered or unmodified wild-type cell.

17. The hypoimmunogenic T cell, non-activated T cell, or population of hypoimmunogenic T cells of claim 16, wherein the hypoimmunogenic T cell, non-activated T cell, or population of hypoimmunogenic T cells does not express a T cell receptor.

18. The hypoimmunogenic T cell, non-activated T cell, or population of hypoimmunogenic T cells of claim 16 or 17, wherein the hypoimmunogenic T cell, non-activated T cell, or population of hypoimmunogenic T cells comprises reduced expression of T cell receptor alpha constant (TRAC) and/or T cell receptor beta constant (TRBC).

19. The hypoimmunogenic T cell, non-activated T cell, or population of hypoimmunogenic T cells of claim 18, wherein the hypoimmunogenic T cell, non-activated T cell, or population of hypoimmunogenic T cells does not express TRAC and/or TRBC.

20. The hypoimmunogenic T cell, non-activated T cell, or population of hypoimmunogenic T cells of any one of claims 1-19, further comprising a second exogenous polynucleotide encoding one or more chimeric antigen receptors (CARs).

21. The hypoimmunogenic T cell, non-activated T cell, or population of hypoimmunogenic T cells of claim 20, wherein the one or more CARs are selected from the group consisting of a CD19-specific CAR, such that the cell is a CD19 CAR T cell, a CD20-specific CAR, such that the cell is a CD20 CAR T cell, a CD22-specific CAR, such that the cell is a CD22 CAR T cell, and a BCMA-specific CAR such that the cell is a BCMA CAR T cell, or a combination thereof.

22. The hypoimmunogenic T cell, non-activated T cell, or population of hypoimmunogenic T cells of claim 21, wherein the hypoimmunogenic T cell, non-activated T cell, or population of hypoimmunogenic T cells comprises a CD19-specific CAR and a CD22-specific CAR such that the cell is a CD19/CD22 CAR T cell.

23. The hypoimmunogenic T cell, non-activated T cell, or population of hypoimmunogenic T cells of claim 22, wherein the CD19-specific CAR and the CD22-specific CAR are encoded by a single bicistronic polynucleotide.

24. The hypoimmunogenic T cell, non-activated T cell, or population of hypoimmunogenic T cells of claim 22, wherein the CD19-specific CAR and the CD22-specific CAR are encoded by two separate polynucleotides.

25. The hypoimmunogenic T cell, non-activated T cell, or population of hypoimmunogenic T cells of any one of claims 1-24, wherein the first and/or second exogenous polynucleotides are inserted into a specific locus of at least one allele of the cell.

26. The hypoimmunogenic T cell, non-activated T cell, or population of hypoimmunogenic T cells of claim 25, wherein the specific locus is selected from the group consisting of a safe harbor locus, an RHD locus, a B2M locus, a CIITA locus, a TRAC locus, and a TRB locus.

27. The hypoimmunogenic T cell, non-activated T cell, or population of hypoimmunogenic T cells of any one of claims 1-26, wherein the polynucleotide encoding CD47 is introduced to the hypoimmunogenic T cell, non-activated T cell, or population of hypoimmunogenic T cells ex vivo from a donor subject.

28. The hypoimmunogenic T cell, non-activated T cell, or population of hypoimmunogenic T cells of claim 27, wherein the polynucleotide encoding CD47 is introduced to the hypoimmunogenic T cell, non-activated T cell, or population of hypoimmunogenic T cells using a lentiviral vector.

29. The hypoimmunogenic T cell, non-activated T cell, or population of hypoimmunogenic T cells of any one of claims 1-26, wherein the polynucleotide encoding CD47 is introduced to the hypoimmunogenic T cell, non-activated T cell, or population of hypoimmunogenic T cells in vivo in the recipient patient.

30. The hypoimmunogenic T cell, non-activated T cell, or population of hypoimmunogenic T cells of claim 29, wherein the exogenous polynucleotide encoding CD47 is introduced to the hypoimmunogenic T cell, non-activated T cell, or population of hypoimmunogenic T cells by contacting the recipient patient with a composition comprising lentiviral vectors comprising (i) a CD4 binding agent or a CD8 binding agent, and (ii) a polynucleotide encoding CD47, wherein the hypoimmunogenic T cell, non-activated T cell, or population of hypoimmunogenic T cells of the recipient patient are transduced with the lentiviral vectors.

31. The hypoimmunogenic T cell, non-activated T cell, or population of hypoimmunogenic T cells of any one of claims 1-26, wherein the polynucleotide encoding CD47 is introduced to the hypoimmunogenic T cell, non-activated T cell, or population of hypoimmunogenic T cells using CRISPR/Cas gene editing.

32. The hypoimmunogenic T cell, non-activated T cell, or population of hypoimmunogenic T cells of claim 31, wherein the CRISPR/Cas gene editing is carried out ex vivo from a donor subject.

33. The hypoimmunogenic T cell, non-activated T cell, or population of hypoimmunogenic T cells of claim 32, wherein the CRISPR/Cas gene editing is carried out using a lentiviral vector.

34. The hypoimmunogenic T cell, non-activated T cell, or population of hypoimmunogenic T cells of claim 31, wherein the CRISPR/Cas gene editing is carried out in vivo in the recipient patient.

35. The hypoimmunogenic T cell, non-activated T cell, or population of hypoimmunogenic T cells of claim 34, wherein the CRISPR/Cas gene editing is carried out by contacting the recipient patient with a composition comprising lentiviral vectors comprising (i) a CD4 binding agent or a CD8 binding agent, (ii) polynucleotides encoding CRISPR/Cas gene editing components, and (iii) a polynucleotide encoding CD47, wherein the hypoimmunogenic T cell, non-activated T cell, or population of hypoimmunogenic T cells of the recipient patient are transduced with the lentiviral vectors.

36. The hypoimmunogenic T cell, non-activated T cell, or population of hypoimmunogenic T cells of any one of claims 20-35, wherein the one or more CARs are introduced to the hypoimmunogenic T cell, non-activated T cell, or population of hypoimmunogenic T cells ex vivo from a donor subject.

37. The hypoimmunogenic T cell, non-activated T cell, or population of hypoimmunogenic T cells of claim 36, wherein the one or more CARs are introduced to the hypoimmunogenic T cell, non-activated T cell, or population of hypoimmunogenic T cells using a lentiviral vector.

38. The hypoimmunogenic T cell, non-activated T cell, or population of hypoimmunogenic T cells of any one of claims 20-35, wherein the one or more CARs are introduced to the hypoimmunogenic T cell, non-activated T cell, or population of hypoimmunogenic T cells in vivo in the recipient patient.

39. The hypoimmunogenic T cell, non-activated T cell, or population of hypoimmunogenic T cells of claim 38, wherein the one or more CARs are introduced to the hypoimmunogenic T cell, non-activated T cell, or population of hypoimmunogenic T cells by contacting the recipient patient with a composition comprising lentiviral vectors comprising (i) a CD4 binding agent or a CD8 binding agent, and (ii) one or more polynucleotides encoding the one or more CARs, wherein the hypoimmunogenic T cell, non-activated T cell, or population of hypoimmunogenic T cells of the recipient patient are transduced with the lentiviral vectors.

40. The hypoimmunogenic T cell, non-activated T cell, or population of hypoimmunogenic T cells of any one of claims 20-35, wherein the one or more CARs are introduced to the hypoimmunogenic T cell, non-activated T cell, or population of hypoimmunogenic T cells using CRISPR/Cas gene editing.

41. The hypoimmunogenic T cell, non-activated T cell, or population of hypoimmunogenic T cells of claim 40, wherein the CRISPR/Cas gene editing is carried out ex vivo from a donor subject.

42. The hypoimmunogenic T cell, non-activated T cell, or population of hypoimmunogenic T cells of claim 41, wherein the CRISPR/Cas gene editing is carried out using a lentiviral vector.

43. The hypoimmunogenic T cell, non-activated T cell, or population of hypoimmunogenic T cells of claim 42, wherein the CRISPR/Cas gene editing is carried out in vivo in the recipient patient.

44. The hypoimmunogenic T cell, non-activated T cell, or population of hypoimmunogenic T cells of claim 43, wherein the CRISPR/Cas gene editing is carried out by contacting the recipient patient with a composition comprising lentiviral vectors comprising (i) a CD4 binding agent or a CD8 binding agent, (ii) polynucleotides encoding CRISPR/Cas gene editing components, and (iii) one or more polynucleotides encoding the one or more CARs, wherein the hypoimmunogenic T cell, non-activated T cell, or population of hypoimmunogenic T cells of the recipient patient are transduced with the lentiviral vectors.

45. The hypoimmunogenic T cell, non-activated T cell, or population of hypoimmunogenic T cells of any one of claims 1-44, wherein the hypoimmunogenic T cell, non-activated T cell, or population of hypoimmunogenic T cells is propagated from a primary T cell or a progeny thereof, wherein the primary T cell is isolated from a donor subject that is Rhesus factor (Rh) negative.

46. The hypoimmunogenic T cell, non-activated T cell, or population of hypoimmunogenic T cells of any one of claims 1-44, wherein the hypoimmunogenic T cell, non-activated T cell, or population of hypoimmunogenic T cells is derived from an iPSC or a progeny thereof, wherein the iPSC or a progeny thereof is derived from a host cell isolated from a donor subject that is RhD negative.

47. The hypoimmunogenic T cell, non-activated T cell, or population of hypoimmunogenic T cells of any one of claims 1-44, wherein the hypoimmunogenic T cell, non-activated T cell, or population of hypoimmunogenic T cells is propagated from a primary T cell or a progeny thereof, wherein the primary T cell or a progeny thereof is isolated from a donor subject that is RhD positive and is genetically engineered to have reduced expression of RhD antigen.

48. The hypoimmunogenic T cell, non-activated T cell, or population of hypoimmunogenic T cells of claim 47, wherein the primary T cell or a progeny thereof is genetically engineered to not express RhD antigen.

49. The hypoimmunogenic T cell, non-activated T cell, or population of hypoimmunogenic T cells of any one of claims 1-44, wherein the hypoimmunogenic T cell, non-activated T cell, or population of hypoimmunogenic T cells is derived from an iPSC or a progeny thereof, wherein the iPSC or a progeny thereof is isolated from a donor subject that is RhD positive and is genetically engineered to have reduced expression of RhD antigen.

50. The hypoimmunogenic T cell, non-activated T cell, or population of hypoimmunogenic T cells of claim 49, wherein the iPSC or a progeny thereof is genetically engineered to not express RhD antigen.

51. The hypoimmunogenic T cell, non-activated T cell, or population of hypoimmunogenic T cells of any one of claims 1-50, wherein the hypoimmunogenic T cell, non-activated T cell, or population of hypoimmunogenic T cells is propagated from a pool of primary T cells or progeny thereof, wherein the pool of primary T cells is isolated from one or more donor subjects different from the recipient patient, wherein the one or more donor subjects optionally comprise either one or more subjects that are RhD positive, one or more subjects that are RhD negative, or a mixture of subjects that are RhD positive and subjects that are RhD negative.

52. The hypoimmunogenic T cell, non-activated T cell, or population of hypoimmunogenic T cells of any one of claims 1-50, wherein the hypoimmunogenic T cell, non-activated T cell, or population of hypoimmunogenic T cells is derived from a pool of iPSCs or progeny thereof, wherein the pool of iPSCs is derived from host cells isolated from one or more donor subjects different from the recipient patient, wherein the one or more donor subjects optionally comprise either one or more subjects that are RhD positive, one or more subjects that are RhD negative, or a mixture of subjects that are RhD positive and subjects that are RhD negative.

53. The hypoimmunogenic T cell, non-activated T cell, or population of hypoimmunogenic T cells of any one of claims 1-52, wherein the hypoimmunogenic T cell, non-activated T cell, or population of hypoimmunogenic T cells is genetically engineered to have reduced expression of RhD antigen using CRISPR/Cas gene editing.

54. The hypoimmunogenic T cell, non-activated T cell, or population of hypoimmunogenic T cells of claim 53, wherein the CRISPR/Cas gene editing is carried out ex vivo from a donor subject.

55. The hypoimmunogenic T cell, non-activated T cell, or population of hypoimmunogenic T cells of claim 54, wherein the CRISPR/Cas gene editing is carried out using a lentiviral vector.

56. The hypoimmunogenic T cell, non-activated T cell, or population of hypoimmunogenic T cells of claim 53, wherein the CRISPR/Cas gene editing is carried out in vivo in the recipient patient.

57. The hypoimmunogenic T cell, non-activated T cell, or population of hypoimmunogenic T cells of claim 56, wherein the CRISPR/Cas gene editing is carried out by contacting the recipient patient with a composition comprising lentiviral vectors comprising (i) a CD4 binding agent or a CD8 binding agent, and (ii) polynucleotides encoding CRISPR/Cas gene editing components targeting the RHD locus, wherein the hypoimmunogenic T cell, non-activated T cell, or population of hypoimmunogenic T cells of the recipient patient are transduced with the lentiviral vectors.

58. A pharmaceutical composition comprising one or more hypoimmunogenic T cell, non-activated T cell, or population of hypoimmunogenic T cells of any one of claims 1-57, and a pharmaceutically acceptable additive, carrier, diluent or excipient.

59. The pharmaceutical composition of claim 58, wherein the composition comprises one or more populations of cells selected from the group consisting of a population of hypoimmunogenic T cells, a population of non-activated T cells, a population hypoimmunogenic CD19 CAR T cells, and a population of hypoimmunogenic CD22 CAR T cells, and a pharmaceutically acceptable additive, carrier, diluent or excipient.

60. The hypoimmunogenic T cell, non-activated T cell, or population of hypoimmunogenic T cells of any one of claims 1-57, or the pharmaceutical composition of claim 58 or 59, for use in the treatment of a disorder in a patient, wherein the patient is RhD sensitized.

61. The hypoimmunogenic T cell, non-activated T cell, or population of hypoimmunogenic T cells of any one of claims 1-57, or the pharmaceutical composition of claim 58 or 59, for use in the treatment of a disorder in a patient, wherein the patient is not RhD sensitized.

62. Use of one or more populations of modified T cells for treating a disorder in a recipient patient, wherein the one or more populations of modified T cells are selected from the group consisting of a population of hypoimmunogenic T cells, a population of non-activated T cells, a population hypoimmunogenic CD19 CAR T cells, and a population of hypoimmunogenic CD22 CAR T cells, wherein the modified T cells comprise reduced expression of RhD antigen and MHC class I and/or class II human leukocyte antigens relative to an unaltered or unmodified wild-type cell, and a first exogenous polynucleotide encoding CD47, wherein the modified T cells are propagated from a primary T cell or a progeny thereof, or are derived from an iPSC or a progeny thereof.

63. The use of claim 62, wherein the modified T cells comprise reduced expression of RhD antigen and MHC class I and class II human leukocyte antigens relative to an unaltered or unmodified wild-type cell, and a first exogenous polynucleotide encoding CD47, wherein the modified T cells are propagated from a primary T cell or a progeny thereof, or are derived from an iPSC or a progeny thereof.

64. The use of claim 62 or 63, wherein the modified T cells comprise reduced expression of RHD and B2M and/or CIITA relative to an unaltered or unmodified wild-type cell, and a first exogenous polynucleotide encoding CD47, wherein the modified T cells are propagated from a primary T cell or a progeny thereof, or are derived from an iPSC or a progeny thereof.

65. The use of claim 64, wherein the modified T cells comprise reduced expression of RHD and B2M and CIITA relative to an unaltered or unmodified wild-type cell, and a first exogenous polynucleotide encoding CD47, wherein the modified T cells are propagated from a primary T cell or a progeny thereof, or are derived from an iPSC or a progeny thereof.

66. The use of any one of claims 62-65, wherein the modified T cells do not express RhD antigen, do not express and MHC class I and/or class II human leukocyte antigens, and comprise a first exogenous polynucleotide encoding CD47, wherein the modified T cells are propagated from a primary T cell or a progeny thereof, or are derived from an iPSC or a progeny thereof.

67. The use of claim 66, wherein the modified T cells do not express RhD antigen, do not express MHC class I human leukocyte antigen, do not express MHC class II human leukocyte antigen, and comprise a first exogenous polynucleotide encoding CD47, wherein the modified T cells are propagated from a primary T cell or a progeny thereof, or are derived from an iPSC or a progeny thereof.

68. The use of claim 65 or 66, wherein the modified T cells do not express RHD, do not express B2M and/or CIITA, and comprise a first exogenous polynucleotide encoding CD47, wherein the modified T cells are propagated from a primary T cell or a progeny thereof, or are derived from an iPSC or a progeny thereof.

69. The use of claim 68, wherein the modified T cells do not express RHD, do not express B2M, do not express CIITA, and comprise a first exogenous polynucleotide encoding CD47, wherein the modified T cells are propagated from a primary T cell or a progeny thereof, or are derived from an iPSC or a progeny thereof.

70. The use of any one of claims 62-69, wherein reduced or lack of expression of RhD antigen is caused by a knock out of the RHD gene.

71. The use of any one of claims 62-70, wherein the modified T cells further comprise reduced expression of a T cell receptor relative to an unaltered or unmodified wild-type cell.

72. The use of claim 71, wherein the modified T cells do not express a T cell receptor.

73. The use of claim 71 or 72, wherein the modified T cells comprise reduced expression of TRAC and/or TRBC.

74. The use of claim 73, wherein the modified T cells do not express TRAC and/or TRBC.

75. The use of any one of claims 62-74, wherein the modified T cells further comprise a second exogenous polynucleotide encoding one or more CARs.

76. The use of claim 75, wherein the one or more CARs are selected from the group consisting of a CD19-specific CAR, such that the cell is a CD19 CAR T cell, a CD20-specific CAR, such that the cell is a CD20 CAR T cell, a CD22-specific CAR, such that the cell is a CD22 CAR T cell, and a BCMA-specific CAR such that the cell is a BCMA CAR T cell, or a combination thereof.

77. The use of claim 76, wherein the hypoimmunogenic T cell, non-activated T cell, or population of hypoimmunogenic T cells comprises a CD19-specific CAR and a CD22-specific CAR such that the cell is a CD19/CD22 CAR T cell.

78. The use of claim 77, wherein the CD19-specific CAR and the CD22-specific CAR are encoded by a single bicistronic polynucleotide.

79. The use of claim 77, wherein the CD19-specific CAR and the CD22-specific CAR are encoded by two separate polynucleotides.

80. The use of any one of claims 62-79, wherein the first and/or second exogenous polynucleotides are inserted into a specific locus of at least one allele of the cell.

81. The use of claim 80, wherein the specific locus is selected from the group consisting of a safe harbor locus, an RHD locus, a B2M locus, a CIITA locus, a TRAC locus, and a TRB locus.

82. The use of any one of claims 62-81, wherein the polynucleotide encoding CD47 is introduced to the hypoimmunogenic T cell, non-activated T cell, or population of hypoimmunogenic T cells ex vivo from a donor subject.

83. The use of claim 82, wherein the polynucleotide encoding CD47 is introduced to the hypoimmunogenic T cell, non-activated T cell, or population of hypoimmunogenic T cells using a lentiviral vector.

84. The use of any one of claims 62-81, wherein the polynucleotide encoding CD47 is introduced to the hypoimmunogenic T cell, non-activated T cell, or population of hypoimmunogenic T cells in vivo in the recipient patient.

85. The use of claim 84, wherein the exogenous polynucleotide encoding CD47 is introduced to the hypoimmunogenic T cell, non-activated T cell, or population of hypoimmunogenic T cells by contacting the recipient patient with a composition comprising lentiviral vectors comprising (i) a CD4 binding agent or a CD8 binding agent, and (ii) a polynucleotide encoding CD47, wherein the hypoimmunogenic T cell, non-activated T cell, or population of hypoimmunogenic T cells of the recipient patient are transduced with the lentiviral vectors.

86. The use of any one of claims 62-85, wherein the polynucleotide encoding CD47 is introduced to the hypoimmunogenic T cell, non-activated T cell, or population of hypoimmunogenic T cells using CRISPR/Cas gene editing.

87. The use of claim 86, wherein the CRISPR/Cas gene editing is carried out ex vivo from a donor subject.

88. The use of claim 87, wherein the CRISPR/Cas gene editing is carried out using a lentiviral vector.

89. The use of claim 86, wherein the CRISPR/Cas gene editing is carried out in vivo in the recipient patient.

90. The use of claim 89, wherein the CRISPR/Cas gene editing is carried out by contacting the recipient patient with a composition comprising lentiviral vectors comprising (i) a CD4 binding agent or a CD8 binding agent, (ii) polynucleotides encoding CRISPR/Cas gene editing components, and (iii) a polynucleotide encoding CD47, wherein the hypoimmunogenic T cell, non-activated T cell, or population of hypoimmunogenic T cells of the recipient patient are transduced with the lentiviral vectors.

91. The use of any one of claims 75-90, wherein the one or more CARs are introduced to the hypoimmunogenic T cell, non-activated T cell, or population of hypoimmunogenic T cells ex vivo from a donor subject.

92. The use of claim 91, wherein the one or more CARs are introduced to the hypoimmunogenic T cell, non-activated T cell, or population of hypoimmunogenic T cells using a lentiviral vector.

93. The use of any one of claims 75-90, wherein the one or more CARs are introduced to the hypoimmunogenic T cell, non-activated T cell, or population of hypoimmunogenic T cells in vivo in the recipient patient.

94. The use of claim 93, wherein the one or more CARs are introduced to the hypoimmunogenic T cell, non-activated T cell, or population of hypoimmunogenic T cells by contacting the recipient patient with a composition comprising lentiviral vectors comprising (i) a CD4 binding agent or a CD8 binding agent, and (ii) one or more polynucleotides encoding the one or more CARs, wherein the hypoimmunogenic T cell, non-activated T cell, or population of hypoimmunogenic T cells of the recipient patient are transduced with the lentiviral vectors.

95. The use of any one of claims 75-90, wherein the one or more CARs are introduced to the hypoimmunogenic T cell, non-activated T cell, or population of hypoimmunogenic T cells using CRISPR/Cas gene editing.

96. The use of claim 95, wherein the CRISPR/Cas gene editing is carried out ex vivo from a donor subject.

97. The use of claim 96, wherein the CRISPR/Cas gene editing is carried out using a lentiviral vector.

98. The use of claim 95, wherein the CRISPR/Cas gene editing is carried out in vivo in the recipient patient.

99. The use of claim 98, wherein the CRISPR/Cas gene editing is carried out by contacting the recipient patient with a composition comprising lentiviral vectors comprising (i) a CD4 binding agent or a CD8 binding agent, (ii) polynucleotides encoding CRISPR/Cas gene editing components, and (iii) one or more polynucleotides encoding the one or more CARs, wherein the hypoimmunogenic T cell, non-activated T cell, or population of hypoimmunogenic T cells of the recipient patient are transduced with the lentiviral vectors.

100. The use of any one of claims 62-99, wherein the modified T cells are propagated from a primary T cell or a progeny thereof, wherein the primary T cell is isolated from a donor subject that is Rhesus factor (Rh) negative.

101. The use of any one of claims 62-99, wherein the modified T cells are derived from an iPSC or a progeny thereof, wherein the iPSC or a progeny thereof is derived from a host cell isolated from a donor subject that is RhD negative.

102. The use of any one of claims 62-99, wherein the modified T cells are propagated from a primary T cell or a progeny thereof, wherein the primary T cell or a progeny thereof is isolated from a donor subject that is RhD positive and is genetically engineered to have reduced expression of RhD antigen.

103. The use of claim 102, wherein the primary T cell or a progeny thereof is genetically engineered to not express RhD antigen.

104. The use of any one of claims 62-99, wherein the modified T cells are derived from an iPSC or a progeny thereof, wherein the iPSC or a progeny thereof is isolated from a donor subject that is RhD positive and is genetically engineered to have reduced expression of RhD antigen.

105. The use of claim 104, wherein the iPSC or a progeny thereof is genetically engineered to not express RhD antigen.

106. The use of any one of claims 62-105, wherein the modified T cells are propagated from a pool of primary T cells or progeny thereof, wherein the pool of primary T cells is isolated from one or more donor subjects different from the recipient patient, wherein the one or more donor subjects optionally comprise either one or more subjects that are RhD positive, one or more subjects that are RhD negative, or a mixture of subjects that are RhD positive and subjects that are RhD negative.

107. The use of any one of claims 62-105, wherein the modified T cells are derived from a pool of iPSCs or progeny thereof, wherein the pool of iPSCs is derived from host cells isolated from one or more donor subjects different from the recipient patient, wherein the one or more donor subjects optionally comprise either one or more subjects that are RhD positive, one or more subjects that are RhD negative, or a mixture of subjects that are RhD positive and subjects that are RhD negative.

108. The use of any one of claims 62-107, wherein the modified T cells are genetically engineered to have reduced expression of RhD antigen using CRISPR/Cas gene editing.

109. The use of claim 108, wherein the CRISPR/Cas gene editing is carried out ex vivo from a donor subject.

110. The use of claim 109, wherein the CRISPR/Cas gene editing is carried out using a lentiviral vector.

111. The use of claim 108, wherein the CRISPR/Cas gene editing is carried out in vivo in the recipient patient.

112. The use of claim 111, wherein the CRISPR/Cas gene editing is carried out by contacting the recipient patient with a composition comprising lentiviral vectors comprising (i) a CD4 binding agent or a CD8 binding agent, and (ii) polynucleotides encoding CRISPR/Cas gene editing components targeting the RHD locus, wherein the modified T cells are transduced with the lentiviral vectors.

113. The use of any one of claims 62-112, wherein the patient is RhD sensitized.

114. The use of any one of claims 62-112, wherein the patient is not RhD sensitized.

115. A method for treating a cancer or a disorder in a recipient patient, comprising administering to the patient a therapeutically effective amount of one or more populations of modified T cells, wherein the one or more populations of modified T cells are selected from the group consisting of a population of hypoimmunogenic T cells, a population of non-activated T cells, a population hypoimmunogenic CD19 CAR T cells, and a population of hypoimmunogenic CD22 CAR T cells, wherein the modified T cells comprise reduced expression of RhD antigen and MHC class I and/or class II human leukocyte antigens relative to an unaltered or unmodified wild-type cell, and a first exogenous polynucleotide encoding CD47, wherein the modified T cells are propagated from a primary T cell or a progeny thereof, or are derived from an iPSC or a progeny thereof.

116. The method of claim 115, wherein the modified T cells comprise reduced expression of RhD antigen and MHC class I and class II human leukocyte antigens relative to an unaltered or unmodified wild-type cell, and a first exogenous polynucleotide encoding CD47, wherein the modified T cells are propagated from a primary T cell or a progeny thereof, or are derived from an iPSC or a progeny thereof.

117. The method of claim 115 or 116, wherein the modified T cells comprise reduced expression of RHD and B2M and/or CIITA relative to an unaltered or unmodified wild-type cell, and a first exogenous polynucleotide encoding CD47, wherein the modified T cells are propagated from a primary T cell or a progeny thereof, or are derived from an iPSC or a progeny thereof.

118. The method of claim 117, wherein the modified T cells comprise reduced expression of RHD and B2M and CIITA relative to an unaltered or unmodified wild-type cell, and a first exogenous polynucleotide encoding CD47, wherein the modified T cells are propagated from a primary T cell or a progeny thereof, or are derived from an iPSC or a progeny thereof.

119. The method of any one of claims 115-118, wherein the modified T cells do not express RhD antigen, do not express and MHC class I and/or class II human leukocyte antigens, and comprise a first exogenous polynucleotide encoding CD47, wherein the modified T cells are propagated from a primary T cell or a progeny thereof, or are derived from an iPSC or a progeny thereof.

120. The method of claim 119, wherein the modified T cells do not express RhD antigen, do not express MHC class I human leukocyte antigen, do not express MHC class II human leukocyte antigen, and comprise a first exogenous polynucleotide encoding CD47, wherein the modified T cells are propagated from a primary T cell or a progeny thereof, or are derived from an iPSC or a progeny thereof.

121. The method of claim 119 or 120, wherein the modified T cells do not express RHD, do not express B2M and/or CIITA, and comprise a first exogenous polynucleotide encoding CD47, wherein the modified T cells are propagated from a primary T cell or a progeny thereof, or are derived from an iPSC or a progeny thereof.

122. The method of claim 121, wherein the modified T cells do not express RHD, do not express B2M, do not express CIITA, and comprise a first exogenous polynucleotide encoding CD47, wherein the modified T cells are propagated from a primary T cell or a progeny thereof, or are derived from an iPSC or a progeny thereof.

123. A method for expanding T cells capable of recognizing and killing tumor cells in a patient, comprising administering to the patient a therapeutically effective amount of one or more populations of modified T cells, wherein the one or more populations of modified T cells are selected from the group consisting of a population of hypoimmunogenic T cells, a population of non-activated T cells, a population hypoimmunogenic CD19 CAR T cells, and a population of hypoimmunogenic CD22 CAR T cells, wherein the modified T cells comprise reduced expression of RhD antigen and MHC class I and/or class II human leukocyte antigens relative to an unaltered or unmodified wild-type cell, and a first exogenous polynucleotide encoding CD47, wherein the modified T cells are propagated from a primary T cell or a progeny thereof, or are derived from an iPSC or a progeny thereof.

124. The method of claim 123, wherein the modified T cells comprise reduced expression of RhD antigen and MHC class I and class II human leukocyte antigens relative to an unaltered or unmodified wild-type cell, and a first exogenous polynucleotide encoding CD47, wherein the modified T cells are propagated from a primary T cell or a progeny thereof, or are derived from an iPSC or a progeny thereof.

125. The method of claim 123 or 124, wherein the modified T cells comprise reduced expression of RHD and B2M and/or CIITA relative to an unaltered or unmodified wild-type cell, and a first exogenous polynucleotide encoding CD47, wherein the modified T cells are propagated from a primary T cell or a progeny thereof, or are derived from an iPSC or a progeny thereof.

126. The method of claim 125, wherein the modified T cells comprise reduced expression of RHD and B2M and CIITA relative to an unaltered or unmodified wild-type cell, and a first exogenous polynucleotide encoding CD47, wherein the modified T cells are propagated from a primary T cell or a progeny thereof, or are derived from an iPSC or a progeny thereof.

127. The method of any one of claims 123-126, wherein the modified T cells do not express RhD antigen, do not express and MHC class I and/or class II human leukocyte antigens, and comprise a first exogenous polynucleotide encoding CD47, wherein the modified T cells are propagated from a primary T cell or a progeny thereof, or are derived from an iPSC or a progeny thereof.

128. The method of claim 127, wherein the modified T cells do not express RhD antigen, do not express MHC class I human leukocyte antigen, do not express MHC class II human leukocyte antigen, and comprise a first exogenous polynucleotide encoding CD47, wherein the modified T cells are propagated from a primary T cell or a progeny thereof, or are derived from an iPSC or a progeny thereof.

129. The method of claim 127 or 128, wherein the modified T cells do not express RHD, do not express B2M and/or CIITA, and comprise a first exogenous polynucleotide encoding CD47, wherein the modified T cells are propagated from a primary T cell or a progeny thereof, or are derived from an iPSC or a progeny thereof.

130. The method of claim 129, wherein the modified T cells do not express RHD, do not express B2M, do not express CIITA, and comprise a first exogenous polynucleotide encoding CD47, wherein the modified T cells are propagated from a primary T cell or a progeny thereof, or are derived from an iPSC or a progeny thereof.

131. The method of any one of claims 115-130, wherein reduced or lack of expression of RhD antigen is caused by a knock out of the RHD gene.

132. The method of any one of claims 115-131, wherein the modified T cells further comprise reduced expression of a T cell receptor relative to an unaltered or unmodified wild-type cell.

133. The method of claim 132, wherein the modified T cells do not express a T cell receptor.

134. The method of claim 132 or 133, wherein the modified T cells comprise reduced expression of TRAC and/or TRBC.

135. The method of claim 134, wherein the modified T cells do not express TRAC and/or TRBC.

136. The method of any one of claims 115-135, wherein the modified T cells further comprise a second exogenous polynucleotide encoding one or more CARs.

137. The method of claim 136, wherein the one or more CARs are selected from the group consisting of a CD19-specific CAR, such that the cell is a CD19 CAR T cell, a CD20-specific CAR, such that the cell is a CD20 CAR T cell, a CD22-specific CAR, such that the cell is a CD22 CAR T cell, and a BCMA-specific CAR such that the cell is a BCMA CAR T cell, or a combination thereof.

138. The method of claim 137, wherein the hypoimmunogenic T cell, non-activated T cell, or population of hypoimmunogenic T cells comprises a CD19-specific CAR and a CD22-specific CAR such that the cell is a CD19/CD22 CAR T cell.

139. The method of claim 138, wherein the CD19-specific CAR and the CD22-specific CAR are encoded by a single bicistronic polynucleotide.

140. The method of claim 138, wherein the CD19-specific CAR and the CD22-specific CAR are encoded by two separate polynucleotides.

141. The method of any one of claims 115-140, wherein the first and/or second exogenous polynucleotides are inserted into a specific locus of at least one allele of the cell.

142. The method of claim 141, wherein the specific locus is selected from the group consisting of a safe harbor locus, an RHD locus, a B2M locus, a CIITA locus, a TRAC locus, and a TRB locus.

143. The method of any one of claims 115-142, wherein the polynucleotide encoding CD47 is introduced to the hypoimmunogenic T cell, non-activated T cell, or population of hypoimmunogenic T cells ex vivo from a donor subject.

144. The method of claim 143, wherein the polynucleotide encoding CD47 is introduced to the hypoimmunogenic T cell, non-activated T cell, or population of hypoimmunogenic T cells using a lentiviral vector.

145. The method of any one of claims 115-142, wherein the polynucleotide encoding CD47 is introduced to the hypoimmunogenic T cell, non-activated T cell, or population of hypoimmunogenic T cells in vivo in the recipient patient.

146. The method of claim 145, wherein the exogenous polynucleotide encoding CD47 is introduced to the hypoimmunogenic T cell, non-activated T cell, or population of hypoimmunogenic T cells by contacting the recipient patient with a composition comprising lentiviral vectors comprising (i) a CD4 binding agent or a CD8 binding agent, and (ii) a polynucleotide encoding CD47, wherein the hypoimmunogenic T cell, non-activated T cell, or population of hypoimmunogenic T cells of the recipient patient are transduced with the lentiviral vectors.

147. The method of any one of claims 115-146, wherein the polynucleotide encoding CD47 is introduced to the hypoimmunogenic T cell, non-activated T cell, or population of hypoimmunogenic T cells using CRISPR/Cas gene editing.

148. The method of claim 147, wherein the CRISPR/Cas gene editing is carried out ex vivo from a donor subject.

149. The method of claim 147, wherein the CRISPR/Cas gene editing is carried out in vivo in the recipient patient.

150. The method of claim 149, wherein the CRISPR/Cas gene editing is carried out by contacting the recipient patient with a composition comprising lentiviral vectors comprising (i) a CD4 binding agent or a CD8 binding agent, (ii) polynucleotides encoding CRISPR/Cas gene editing components, and (iii) a polynucleotide encoding CD47, wherein the hypoimmunogenic T cell, non-activated T cell, or population of hypoimmunogenic T cells of the recipient patient are transduced with the lentiviral vectors.

151. The method of any one of claims 136-150, wherein the one or more CARs are introduced to the hypoimmunogenic T cell, non-activated T cell, or population of hypoimmunogenic T cells ex vivo from a donor subject.

152. The method of claim 151, wherein the one or more CARs are introduced to the hypoimmunogenic T cell, non-activated T cell, or population of hypoimmunogenic T cells using a lentiviral vector.

153. The method of any one of claims 136-150, wherein the one or more CARs are introduced to the hypoimmunogenic T cell, non-activated T cell, or population of hypoimmunogenic T cells in vivo in the recipient patient.

154. The method of claim 153, wherein the one or more CARs are introduced to the hypoimmunogenic T cell, non-activated T cell, or population of hypoimmunogenic T cells by contacting the recipient patient with a composition comprising lentiviral vectors comprising (i) a CD4 binding agent or a CD8 binding agent, and (ii) one or more polynucleotides encoding the one or more CARs, wherein the hypoimmunogenic T cell, non-activated T cell, or population of hypoimmunogenic T cells of the recipient patient are transduced with the lentiviral vectors.

155. The method of any one of claims 136-150, wherein the one or more CARs are introduced to the hypoimmunogenic T cell, non-activated T cell, or population of hypoimmunogenic T cells using CRISPR/Cas gene editing.

156. The method of claim 155, wherein the CRISPR/Cas gene editing is carried out ex vivo from a donor subject.

157. The method of claim 156, wherein the CRISPR/Cas gene editing is carried out using a lentiviral vector.

158. The method of claim 155, wherein the CRISPR/Cas gene editing is carried out in vivo in the recipient patient.

159. The method of claim 158, wherein the CRISPR/Cas gene editing is carried out by contacting the recipient patient with a composition comprising lentiviral vectors comprising (i) a CD4 binding agent or a CD8 binding agent, (ii) polynucleotides encoding CRISPR/Cas gene editing components, and (iii) one or more polynucleotides encoding the one or more CARs, wherein the hypoimmunogenic T cell, non-activated T cell, or population of hypoimmunogenic T cells of the recipient patient are transduced with the lentiviral vectors.

160. The method of any one of claims 115-159, wherein the modified T cells are propagated from a primary T cell or a progeny thereof, wherein the primary T cell is isolated from a donor subject that is Rhesus factor (Rh) negative.

161. The method of any one of claims 115-159, wherein the modified T cells are derived from an iPSC or a progeny thereof, wherein the iPSC or a progeny thereof is derived from a host cell isolated from a donor subject that is RhD negative.

162. The method of any one of claims 115-159, wherein the modified T cells are propagated from a primary T cell or a progeny thereof, wherein the primary T cell or a progeny thereof is isolated from a donor subject that is RhD positive and is genetically engineered to have reduced expression of RhD antigen.

163. The method of claim 162, wherein the primary T cell or a progeny thereof is genetically engineered to not express RhD antigen.

164. The method of any one of claims 115-159, wherein the modified T cells are derived from an iPSC or a progeny thereof, wherein the iPSC or a progeny thereof is isolated from a donor subject that is RhD positive and is genetically engineered to have reduced expression of RhD antigen.

165. The method of claim 164, wherein the iPSC or a progeny thereof is genetically engineered to not express RhD antigen.

166. The method of any one of claims 115-165, wherein the modified T cells are propagated from a pool of primary T cells or progeny thereof, wherein the pool of primary T cells is isolated from one or more donor subjects different from the recipient patient, wherein the one or more donor subjects optionally comprise either one or more subjects that are RhD positive, one or more subjects that are RhD negative, or a mixture of subjects that are RhD positive and subjects that are RhD negative.

167. The method of any one of claims 115-165, wherein the modified T cells are derived from a pool of iPSCs or progeny thereof, wherein the pool of iPSCs is derived from host cells isolated from one or more donor subjects different from the recipient patient, wherein the one or more donor subjects optionally comprise either one or more subjects that are RhD positive, one or more subjects that are RhD negative, or a mixture of subjects that are RhD positive and subjects that are RhD negative.

168. The method of any one of claims 115-167, wherein the modified T cells are genetically engineered to have reduced expression of RhD antigen using CRISPR/Cas gene editing.

169. The method of claim 168, wherein the CRISPR/Cas gene editing is carried out ex vivo from a donor subject.

170. The method of claim 169, wherein the CRISPR/Cas gene editing is carried out using a lentiviral vector.

171. The method of claim 168, wherein the CRISPR/Cas gene editing is carried out in vivo in the recipient patient.

172. The method of claim 171, wherein the CRISPR/Cas gene editing is carried out by contacting the recipient patient with a composition comprising lentiviral vectors comprising (i) a CD4 binding agent or a CD8 binding agent, and (ii) polynucleotides encoding CRISPR/Cas gene editing components targeting the RHD locus, wherein the cells are transduced with the lentiviral vectors.

173. The method of any one of claims 115-172, wherein the patient is RhD sensitized.

174. The method of any one of claims 115-172, wherein the patient is not RhD sensitized.

175. The method of any one of claims 115-174, wherein upon administration, the one or more populations of modified T cells elicits a reduced level of immune activation or no immune activation in the patient.

176. The method of any one of claims 115-175, wherein upon administration, the one or more populations of modified T cells elicits a reduced level of systemic TH1 activation or no systemic TH1 activation in the patient.

177. The method of any one of claims 115-176, wherein upon administration, the one or more populations of modified T cells elicits a reduced level of immune activation of peripheral blood mononuclear cells (PBMCs) or no immune activation of PBMCs in the patient.

178. The method of any one of claims 115-177, wherein upon administration, the one or more populations of modified T cells elicits a reduced level of donor-specific IgG antibodies or no donor specific IgG antibodies against the hypoimmunogenic T cells in the patient.

179. The method of any one of claims 115-178, wherein upon administration, the one or more populations of modified T cells elicits a reduced level of IgM and IgG antibody production or no IgM and IgG antibody production against the hypoimmunogenic T cells in the patient.

180. The method of any one of claims 115-179, wherein upon administration, the one or more populations of modified T cells elicits a reduced level of cytotoxic T cell killing or no cytotoxic T cell killing of the hypoimmunogenic T cells in the patient.

181. The method of any one of claims 115-180, wherein the patient is not administered an immunosuppressive agent at least 3 days or more before or after the administration of the population of hypoimmunogenic T cells.

182. A method of modifying a hypoimmunogenic T cell such that the modified hypoimmunogenic T cell comprises reduced expression of RhD antigen relative to an unaltered or unmodified wild-type cell, the method comprising contacting a hypoimmunogenic T cell with a composition comprising lentiviral vectors comprising (i) a CD4 binding agent or a CD8 binding agent, and (ii) polynucleotides encoding CRISPR/Cas gene editing components targeting the RHD locus, wherein the hypoimmunogenic T cell is transduced with the lentiviral vectors, the hypoimmunogenic T cell is propagated from a primary T cell or a progeny thereof, or is derived from an iPSC or a progeny thereof, and the hypoimmunogenic T cell comprises reduced expression of MHC class I and/or class II human leukocyte antigens relative to an unaltered or unmodified wild-type cell and a first exogenous polynucleotide encoding CD47.

183. The method of claim 182, wherein the lentiviral vectors further comprise (iii) one or more polynucleotides encoding one or more CARs.

184. The method of claim 183, wherein the polynucleotide encoding the one or more CARs is inserted into the RHD locus of the modified hypoimmunogenic T cell.

185. The method of claim 184, wherein the contacting of the hypoimmunogenic T cell is carried out ex vivo from a donor subject.

186. The method of claim 185, wherein the contacting of the hypoimmunogenic T cell is carried out using a lentiviral vector.

187. The method of claim 184, wherein the contacting of the hypoimmunogenic T cell is carried out in vivo in a recipient patient.

188. The method of any one of claims 182-187, wherein the recipient patient has a disease or condition.
